(19) United States
(12) Patent Application Publication
(10) Pub. No.: US 2024/0239811 A1
(43) Pub. Date: Jul. 18, 2024

(54) 5-(5-(PIPERIDIN-4-YL)THIENO[3,2-C]PYRAZOL-2-YL]INDAZOLE DERIVATIVES AND RELATED COMPOUNDS AS MODULATORS FOR SPLICING NUCLEIC ACIDS AND FOR THE T

(71) Applicant: REMIX THERAPEUTICS INC., Cambridge, MA (US)

(72) Inventors: Dominic Reynolds, Stoneham, MA (US); Michael W. Seiler, Belmont, MA (US); Anant A. Agrawal, Waltham, MA (US); Frederic Vaillancourt, Newton, MA (US); Peter Smith, Arlington, MA (US); Stepan Vyskocil, Benesov (CZ); Allen T. Hopper, Lexington, MA (US); Sudeep Prajapati, Somerville, MA (US)

(21) Appl. No.: 18/014,057

(22) PCT Filed: Jul. 2, 2021
(86) PCT No.: PCT/US2021/040352

§ 371 Date: Dec. 30, 2022

Related U.S. Application Data
(60) Provisional application No. 63/135,332, filed on Jan. 8, 2021.
(60) Provisional application No. 63/126,320, filed on Dec. 16, 2020.
(60) Provisional application No. 63/072,871, filed on Aug. 31, 2020.
(60) Provisional application No. 63/047,900, filed on Jul. 2, 2020.

(51) Int. Cl. C07D 513/04; A61K 31/4162; A61K 31/429; A61K 31/454; A61K 31/4545; A61K 31/496; A61K 31/4985; A61K 31/499; A61K 31/5025; C07D 495/04; C07D 519/00

(52) U.S. Cl. CPC C07D 513/04; A61K 31/4162; A61K 31/429; A61K 31/454; A61K 31/4545; A61K 31/496; A61K 31/4985; A61K 31/499; A61K 31/5025; C07D 495/04; C07D 519/00

(57) ABSTRACT

The present disclosure features compounds e.g. of formula (I-a) and similar compounds e.g. of formulae (1-g), (1-i), (1-j), (III), (III-a), (III-b) and (IV) disclosed herein and other related compounds, and pharmaceutical compositions thereof. The present disclosure further discloses said compounds and their compositions for use in methods of modulating nucleic acid splicing, as well as said compounds for use in methods of treating e.g.: ·proliferative diseases, such as e.g. cancer, benign neoplasms or angiogenesis, ·neurological diseases or disorders, such as e.g. Huntington's disease, ·autoimmune diseases or disorders, immunodeficiency diseases or disorders, lysosomal storage disease or disorder, cardiovascular diseases or disorders, metabolic diseases or disorders, respiratory diseases or disorders, renal diseases or disorders, or infectious diseases. Exemplary compounds are e.g. 5-[5-(piperidin-4-yl)thieno[3,2-c]pyrazol-2-yl]indazole and 5-[2-(piperidin-4-yl)-[1,2,4]triazolo[3,2-b] [1,3]thiazol-5-yl]indazole derivatives and related compounds.

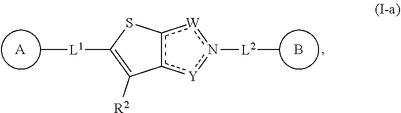

### CLAIM OF PRIORITY

This application claims priority to U.S. Application No. 63/047,900, filed Jul. 2, 2020; U.S. Application No. 63/072,871, filed Aug. 31, 2020; and U.S. Application No. 63/126,320, filed Dec. 16, 2020, and U.S. Application No. 63/135,332, filed Jan. 8, 2021. The disclosure of each of the foregoing applications is incorporated herein by reference in its entirety.

### BACKGROUND

Alternative splicing is a major source of protein diversity in higher eukaryotes, and is frequently regulated in a tissue-specific or development stage-specific manner. Disease associated alternative splicing patterns in pre-mRNAs are often mapped to changes in splice site signals or sequence motifs and regulatory splicing factors (Faustino and Cooper (2003), Genes Dev 17(4):419-37). Current therapies to modulate RNA expression involve oligonucleotide targeting and gene therapy; however, each of these modalities exhibit unique challenges as currently presented. As such, there is a need for new technologies to modulate RNA expression, including the development of small molecule compounds that target splicing.

### SUMMARY

The present disclosure features compounds and related compositions that, inter alia, modulate nucleic acid splicing, e.g., splicing of a pre-mRNA, as well as methods of use thereof. In an embodiment, the compounds described herein are compounds of Formula (I), (II), (III), or (IV), and pharmaceutically acceptable salts, solvates, hydrates, tautomers, or stereoisomers thereof. The present disclosure additionally provides methods of using the compounds of the disclosure (e.g., compounds of Formulas (I), (II), (III), and (IV), and pharmaceutically acceptable salts, solvates, hydrates, tautomers, stereoisomers thereof), and compositions thereof, e.g., to target, and in embodiments bind or form a complex with, a nucleic acid (e.g., a pre-mRNA or nucleic acid component of a small nuclear ribonucleoprotein (snRNP) or spliceosome), a protein (e.g., a protein component of an snRNP or spliceosome, e.g., a member of the splicing machinery, e.g., one or more of the U1, U2, U4, U5, U6, U11, U12, U4atac, U6atac snRNPs), or a combination thereof. In another aspect, the compounds described herein may be used to alter the composition of a nucleic acid (e.g., a pre-mRNA or mRNA (e.g., a pre-mRNA and the mRNA which arises from the pre-mRNA), e.g., by increasing or decreasing splicing at a splice site. In some embodiments, increasing or decreasing splicing results in modulating the level of a gene product (e.g., an RNA or protein) produced.

In another aspect, the compounds described herein may be used for the prevention and/or treatment of a disease, disorder, or condition, e.g., a disease, disorder or condition associated with splicing, e.g., alternative splicing. In some embodiments, the compounds described herein (e.g., compounds of Formulas (I), (II), (III), (IV), and pharmaceutically acceptable salts, solvates, hydrates, tautomers, stereoisomers thereof) and compositions thereof are used for the prevention and/or treatment of a proliferative disease, disorder, or condition (e.g., a disease, disorder, or condition characterized by unwanted cell proliferation, e.g., a cancer or a benign neoplasm) in a subject. In some embodiments, the compounds described herein (e.g., compounds of Formulas (I), (II), (III), (IV), and pharmaceutically acceptable salts, solvates, hydrates, tautomers, stereoisomers thereof) and compositions thereof are used for the prevention and/or treatment of a non-proliferative disease, disorder, or condition. In some embodiments, the compounds described herein (e.g., compounds of Formulas (I), (II), (III), (IV), and pharmaceutically acceptable salts, solvates, hydrates, tautomers, stereoisomers thereof) and compositions thereof are used for the prevention and/or treatment of a neurological disease or disorder, an autoimmune disease or disorder, immunodeficiency disease or disorder, a lysosomal storage disease or disorder, a cardiovascular disease or disorder, a metabolic disease or disorder, a respiratory disease or disorder, a renal disease or disorder, or an infectious disease in a subject.

In one aspect, the resent disclosure provides compounds of Formula (I):

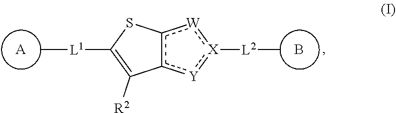

or a pharmaceutically acceptable salt, solvate, hydrate, tautomer, or stereoisomer thereof, wherein each of A, B, L¹, L², W, X, Y, R², and subvariables thereof are defined as described herein.

In another aspect, the present disclosure provides compounds of Formula (II):

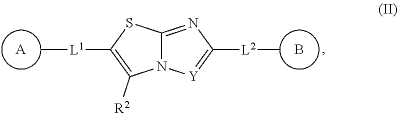

or a pharmaceutically acceptable salt, solvate, hydrate,
tautomer, or stereoisomer thereof, wherein each of A, B, L¹, L², Y, R², and subvariables thereof are defined as described herein.

In another aspect, the present disclosure provides compounds of Formula (III):

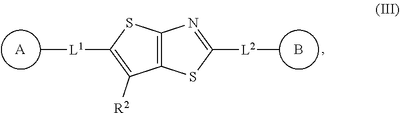

or a pharmaceutically acceptable salt, solvate, hydrate,
tautomer, or stereoisomer thereof, wherein each of A, B, L¹, L², R², and subvariables thereof are defined as described herein.

In another aspect, the present disclosure provides compounds of Formula (IV):

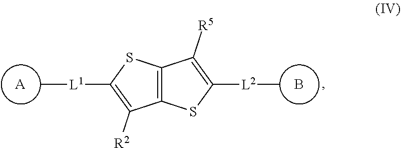

or a pharmaceutically acceptable salt, solvate, hydrate,
tautomer, or stereoisomer thereof, wherein each of A, B, L¹, L², R², R⁵, and subvariables thereof are defined as described herein.

In another aspect, the present invention provides pharmaceutical compositions comprising a compound of Formula (I), (II), (III), or (IV), or a pharmaceutically acceptable salt, solvate, hydrate, tautomer, or stereoisomer thereof, and optionally a pharmaceutically acceptable excipient. In an embodiment, the pharmaceutical compositions described herein include a therapeutically effective amount of a compound of Formula (I), (II), (III), or (IV), or a pharmaceutically acceptable salt, solvate, hydrate, tautomer, or stereoisomer thereof.

In another aspect, the present disclosure provides methods for modulating splicing, e.g., splicing of a nucleic acid (e.g., a DNA or RNA, e.g., a pre-mRNA) with a compound of Formulas (I), (II), (III), or (IV), or a pharmaceutically acceptable salt, solvate, hydrate, tautomer, or stereoisomer thereof. In another aspect, the present disclosure provides compositions for use in modulating splicing, e.g., splicing of a nucleic acid (e.g., a DNA or RNA, e.g., a pre-mRNA) with a compound of Formulas (I), (II), (III), or (IV), or a pharmaceutically acceptable salt, solvate, hydrate, tautomer, or stereoisomer thereof. Modulation of splicing may comprise impacting any step involved in splicing and may include an event upstream or downstream of a splicing event. For example, in some embodiments, the compound of Formulas (I), (II), (III), or (IV), binds to a target, e.g., a target nucleic acid (e.g., DNA or RNA, e.g., a precursor RNA, e.g., a pre-mRNA), a target protein, or combination thereof (e.g., an snRNP and a pre-mRNA). A target may include a splice site in a pre-mRNA or a component of the splicing machinery, such as the U1 snRNP. In some embodiments, the compound of Formulas (I), (II), (III), or (IV) alters a target nucleic acid (e.g., DNA or RNA, e.g., a precursor RNA, e.g., a pre-mRNA), target protein, or combination thereof. In some embodiments, the compound of Formulas (I), (II), (III), or (IV) increases or decreases splicing at a splice site on a target nucleic acid (e.g., an RNA, e.g., a precursor RNA, e.g., a pre-mRNA) by about 0.5% or more (e.g., about 1%, 2%, 3%, 4%, 5%, 10%, 20%, 30%, 40%, 50%, 75%, 90%, 95%, or more), relative to a reference (e.g., the absence of a compound of Formulas (I), (II), (III), or (IV), e.g., in a healthy or diseased cell or tissue). In some embodiments, the presence of a compound of Formulas (I), (II), (III), or (IV) results an increase or decrease of transcription of a target nucleic acid (e.g., an RNA) by about 0.5% or more (e.g., about 1%, 2%, 3%, 4%, 5%, 10%, 20%, 30%, 40%, 50%, 75%, 90%, 95%, or more), relative to a reference (e.g., the absence of a compound of Formulas (I), (II), (III), or (IV), e.g., in a healthy or diseased cell or tissue).

In another aspect, the present disclosure provides methods for preventing and/or treating a disease, disorder, or condition in a subject by administering a compound of Formulas (I), (II), (III), or (IV), or a pharmaceutically acceptable salt, solvate, hydrate, tautomer, or stereoisomer thereof, or related compositions. In some embodiments, the disease or disorder entails unwanted or aberrant splicing. In some embodiments, the disease or disorder is a proliferative disease, disorder, or condition. Exemplary proliferative diseases include cancer, a benign neoplasm, or angiogenesis. In other embodiments, the present disclosure provides methods for treating and/or preventing a non-proliferative disease, disorder, or condition. In still other embodiments, the present disclosure provides methods for treating and/or preventing a neurological disease or disorder, autoimmune disease or disorder, immunodeficiency disease or disorder, lysosomal storage disease or disorder, cardiovascular disease or disorder, metabolic disease or disorder, respiratory disease or disorder, renal disease or disorder, or infectious disease.

In another aspect, the present disclosure provides methods of down-regulating the expression of (e.g., the level of or the rate of production of) a target protein with a compound of Formulas (I) or (II), or a pharmaceutically acceptable salt, solvate, hydrate, tautomer, or stereoisomer thereof in a biological sample or subject. In another aspect, the present disclosure provides methods of up-regulating the expression of (e.g., the level of or the rate of production of) a target protein with a compound of Formulas (I) or (II), or a pharmaceutically acceptable salt, solvate, hydrate, tautomer, or stereoisomer thereof in a biological sample or subject. In another aspect, the present disclosure provides methods of altering the isoform of a target protein with a compound of Formulas (I) or (II), or a pharmaceutically acceptable salt, solvate, hydrate, tautomer, or stereoisomer thereof in a biological sample or subject. Another aspect of the disclosure relates to methods of inhibiting the activity of a target protein in a biological sample or subject. In some embodiments, administration of a compound of Formulas (I) or (II) to a biological sample, a cell, or a subject comprises inhibition of cell growth or induction of cell death.

In another aspect, the present disclosure provides compositions for use in preventing and/or treating a disease, disorder, or condition in a subject by administering a compound of Formulas (I) or (II) or a pharmaceutically acceptable salt, solvate, hydrate, tautomer, or stereoisomer thereof, or related compositions. In some embodiments, the disease or disorder entails unwanted or aberrant splicing. In some embodiments, the disease or disorder is a proliferative disease, disorder, or condition. Exemplary proliferative diseases include cancer, a benign neoplasm, or angiogenesis. In other embodiments, the present disclosure provides methods for treating and/or preventing a non-proliferative disease, disorder, or condition. In still other embodiments, the present disclosure provides compositions for use in treating and/or preventing a neurological disease or disorder, autoimmune disease or disorder, immunodeficiency disease or disorder, lysosomal storage disease or disorder, cardiovascular disease or disorder, metabolic disease or disorder, respiratory disease or disorder, renal disease or disorder, or infectious disease.

In another aspect, the present disclosure provides compositions for use in down-regulating the expression of (e.g., the level of or the rate of production of) a target protein with a compound of Formulas (I), (II), (III), or (IV), or a pharmaceutically acceptable salt, solvate, hydrate, tautomer, or stereoisomer thereof in a biological sample or subject. In another aspect, the present disclosure provides compositions for use in up-regulating the expression of (e.g., the level of or the rate of production of) a target protein with a compound of Formulas (I), (II), (III), or (IV), or a pharmaceutically acceptable salt, solvate, hydrate, tautomer, or stereoisomer thereof in a biological sample or subject. In another aspect, the present disclosure provides compositions for use in altering the isoform of a target protein with a compound of Formulas (I), (II), (III), or (IV), or a pharmaceutically acceptable salt, solvate, hydrate, tautomer, or stereoisomer thereof in a biological sample or subject. Another aspect of the disclosure relates to compositions for use in inhibiting the activity of a target protein in a biological sample or subject. In some embodiments, administration of a compound of Formulas (I), (II), (III), or (IV) to a biological sample, a cell, or a subject comprises inhibition of cell growth or induction of cell death.

In another aspect, the present disclosure features kits comprising a container with a compound of Formulas (I), (II), (III), or (IV), or a pharmaceutically acceptable salt, solvate, hydrate, tautomer, stereoisomer thereof, or a pharmaceutical composition thereof. In certain embodiments, the kits described herein further include instructions for administering the compound of Formulas (I), (II), (III), or (IV), or the pharmaceutically acceptable salt, solvate, hydrate, tautomer, stereoisomer thereof, or the pharmaceutical composition thereof.

In any and all aspects of the present disclosure, in some embodiments, the compound, target nucleic acid (e.g., DNA, RNA, e.g., pre-mRNA), or target protein described herein is a compound, target nucleic acid (e.g., DNA, RNA, e.g., pre-mRNA), or target protein other than a compound, target nucleic acid (e.g., DNA, RNA, e.g., pre-mRNA), or target protein described one of U.S. Pat. No. 8,729,263, U.S. Publication No. 2015/0005289, WO 2014/028459, WO 2016/128343, WO 2016/196386, WO 2017/100726, WO 2018/232039, WO 2018/098446, WO 2018/226622, WO 2019/028440, WO 2019/060917, WO 2019/199972, WO 2019/005993, WO 2019/005980, WO 2020/005882, WO 2020/005877, WO 2020/005873 and WO 2020/004594, each of which is incorporated herein by reference in its entirety. In some embodiments, the compound, target nucleic acid (e.g., DNA, RNA, e.g., pre-mRNA), or target protein described herein is a compound, target nucleic acid (e.g., DNA, RNA, e.g., pre-mRNA), or target protein described one of U.S. Pat. No. 8,729,263, U.S. Publication No. 2015/0005289, WO 2014/028459, WO 2016/128343, WO 2016/196386, WO 2017/100726, WO 2018/232039, WO 2018/098446, WO 2018/226622, WO 2019/028440, WO 2019/060917, WO 2019/199972, WO 2019/005993, WO 2019/005980, WO 2020/005882, WO 2020/005877, WO 2020/005873, and WO 2020/004594, each of which is incorporated herein by reference in its entirety.

The details of one or more embodiments of the invention are set forth herein. Other features, objects, and advantages of the invention will be apparent from the Detailed Description, the Examples, and the Claims.

### DETAILED DESCRIPTION

### Selected Chemical Definitions

Definitions of specific functional groups and chemical terms are described in more detail below. The chemical elements are identified in accordance with the Periodic Table of the Elements, CAS version, Handbook of Chemistry and Physics, 75^{th} Ed., inside cover, and specific functional groups are generally defined as described therein. Additionally, general principles of organic chemistry, as well as specific functional moieties and reactivity, are described in Thomas Sorrell, Organic Chemistry, University Science Books, Sausalito, 1999; Smith and March, March's Advanced Organic Chemistry, 5^{TH} Edition, John Wiley & Sons, Inc., New York, 2001; Larock, Comprehensive Organic Transformations, VCH Publishers, Inc., New York, 1989; and Carruthers, Some Modern Methods of Organic Synthesis, 3^{rd} Edition, Cambridge University Press, Cambridge, 1987.

The abbreviations used herein have their conventional meaning within the chemical and biological arts. The chemical structures and formulae set forth herein are constructed according to the standard rules of chemical valency known in the chemical arts.

When a range of values is listed, it is intended to encompass each value and sub-range within the range. For example “C₁-C₆ alkyl” is intended to encompass, C₁, C₂, C₃, C₄, C₅, C₆, C₁-C₆, C₁-C₅, C₁-C₄, C₁-C₃, C₁-C₂, C₂-C₆, C₂-C₅, C₂-C₄, C₂-C₃, C₃-C₆, C₃-C₅, C₃-C₄, C₄-C₆, C₄-C₅, and C₅-C₆ alkyl.

The following terms are intended to have the meanings presented therewith below and are useful in understanding the description and intended scope of the present invention.

As used herein, “alkyl” refers to a radical of a straight-chain or branched saturated hydrocarbon group having from 1 to 24 carbon atoms (“C₁-C₂₄ alkyl”). In some embodiments, an alkyl group has 1 to 12 carbon atoms (“C₁-C₁₂ alkyl”). In some embodiments, an alkyl group has 1 to 8 carbon atoms (“C₁-C₈ alkyl”). In some embodiments, an alkyl group has 1 to 6 carbon atoms (“C₁-C₆ alkyl”). In some embodiments, an alkyl group has 2 to 6 carbon atoms (“C₂-C₆ alkyl”). In some embodiments, an alkyl group has 1 carbon atom (“C₁ alkyl”). Examples of C₁-C₆alkyl groups include methyl (C₁), ethyl (C₂), n-propyl (C₃), isopropyl (C₃), n-butyl (C₄), tert-butyl (C₄), sec-butyl (C₄), iso-butyl (C₄), n-pentyl (C₅), 3-pentanyl (C₅), amyl (C₅), neopentyl (C₅), 3-methyl-2-butanyl (C₅), tertiary amyl (C₅), and n-hexyl (C₆). Additional examples of alkyl groups include n-heptyl (C₇), n-octyl (C₈) and the like. Each instance of an alkyl group may be independently optionally substituted, i.e., unsubstituted (an “unsubstituted alkyl”) or substituted (a “substituted alkyl”) with one or more substituents; e.g., for instance from 1 to 5 substituents, 1 to 3 substituents, or 1 substituent. In certain embodiments, the alkyl group is unsubstituted C₁-C₁₀ alkyl (e.g., —CH₃). In certain embodiments, the alkyl group is substituted C₁-C₆ alkyl.

As used herein, “alkenyl” refers to a radical of a straight-chain or branched hydrocarbon group having from 2 to 24 carbon atoms, one or more carbon-carbon double bonds, and no triple bonds (“C₂-C₂₄ alkenyl”). In some embodiments, an alkenyl group has 2 to 10 carbon atoms (“C₂-C₁₀ alkenyl”). In some embodiments, an alkenyl group has 2 to 8 carbon atoms (“C₂-C₅ alkenyl”). In some embodiments, an alkenyl group has 2 to 6 carbon atoms (“C₂-C₆ alkenyl”). In some embodiments, an alkenyl group has 2 carbon atoms (“C₂ alkenyl”). The one or more carbon-carbon double bonds can be internal (such as in 2-butenyl) or terminal (such as in 1-butenyl). Examples of C₂-C₄ alkenyl groups include ethenyl (C₂), 1-propenyl (C₃), 2-propenyl (C₃), 1-butenyl (C₄), 2-butenyl (C₄), butadienyl (C₄), and the like. Examples of C₂-C₆ alkenyl groups include the aforementioned C₂₋₄ alkenyl groups as well as pentenyl (C₅), pentadienyl (C₅), hexenyl (C₆), and the like. Additional examples of alkenyl include heptenyl (C₇), octenyl (C₈), octatrienyl (C₅), and the like. Each instance of an alkenyl group may be independently optionally substituted, i.e., unsubstituted (an “unsubstituted alkenyl”) or substituted (a “substituted alkenyl”) with one or more substituents e.g., for instance from 1 to 5 substituents, 1 to 3 substituents, or 1 substituent. In certain embodiments, the alkenyl group is unsubstituted C₁-C₁₀ alkenyl. In certain embodiments, the alkenyl group is substituted C₂-C₆ alkenyl.

As used herein, the term “alkynyl” refers to a radical of a straight-chain or branched hydrocarbon group having from 2 to 24 carbon atoms, one or more carbon-carbon triple bonds (“C₂-C₂₄ alkenyl”). In some embodiments, an alkynyl group has 2 to 10 carbon atoms (“C₂-C₁₀ alkynyl”). In some embodiments, an alkynyl group has 2 to 8 carbon atoms (“C₂-C₈ alkynyl”). In some embodiments, an alkynyl group has 2 to 6 carbon atoms (“C₂-C₆ alkynyl”). In some embodiments, an alkynyl group has 2 carbon atoms (“C₂ alkynyl”). The one or more carbon-carbon triple bonds can be internal (such as in 2-butynyl) or terminal (such as in 1-butynyl). Examples of C₂-C₄ alkynyl groups include ethynyl (C₂), 1-propynyl (C₃), 2-propynyl (C₃), 1-butynyl (C₄), 2-butynyl (C₄), and the like. Each instance of an alkynyl group may be independently optionally substituted, i.e., unsubstituted (an “unsubstituted alkynyl”) or substituted (a “substituted alkynyl”) with one or more substituents e.g., for instance from 1 to 5 substituents, 1 to 3 substituents, or 1 substituent. In certain embodiments, the alkynyl group is unsubstituted C₂₋₁₀ alkynyl. In certain embodiments, the alkynyl group is substituted C₂₋₆ alkynyl.

As used herein, the term “haloalkyl,” refers to a non-cyclic stable straight or branched chain, or combinations thereof, including at least one carbon atom and at least one halogen selected from the group consisting of F, Cl, Br, and I. The halogen(s) F, Cl, Br, and I may be placed at any position of the haloalkyl group. Exemplary haloalkyl groups include, but are not limited to: —CF₃, —CCl₃, —CH₂—CF₃, —CH₂—CCl₃, —CH₂—CBr₃, —CH₂—Cl₃, —CH₂—CH₂—CH(CF₃)—CH₃, —CH₂—CH₂—CH(Br)—CH₃, and —CH₂—CH═CH—CH₂—CF₃. Each instance of a haloalkyl group may be independently optionally substituted, i.e., unsubstituted (an “unsubstituted haloalkyl”) or substituted (a “substituted haloalkyl”) with one or more substituents e.g., for instance from 1 to 5 substituents, 1 to 3 substituents, or 1 substituent

As used herein, the term “heteroalkyl,” refers to a non-cyclic stable straight or branched chain, or combinations thereof, including at least one carbon atom and at least one heteroatom selected from the group consisting of O, N, P, Si, and S, and wherein the nitrogen and sulfur atoms may optionally be oxidized, and the nitrogen heteroatom may optionally be quaternized. The heteroatom(s) 0, N, P, S, and Si may be placed at any position of the heteroalkyl group. Exemplary heteroalkyl groups include, but are not limited to: —CH₂—CH₂—O—CH₃, —CH₂—CH₂—NH—CH₃, —CH₂—CH₂—N(CH₃)—CH₃, —CH₂—S—CH₂—CH₃, —CH₂—CH₂, —S(O)—CH₃, —CH₂—CH₂—S(O)₂—CH₃, —CH═CHO—CH₃, —Si(CH₃)₃, —CH₂—CH═N—OCH₃, —CH═CH—N(CH₃)—CH₃, —O—CH₃, and —O—CH₂—CH₃. Up to two or three heteroatoms may be consecutive, such as, for example, —CH₂—NH—OCH₃ and —CH₂—O—Si(CH₃)₃. Where “heteroalkyl” is recited, followed by recitations of specific heteroalkyl groups, such as —CH₂O, —NR^{C}R^{D}, or the like, it will be understood that the terms heteroalkyl and —CH₂O or —NR^{C}R^{D} are not redundant or mutually exclusive. Rather, the specific heteroalkyl groups are recited to add clarity. Thus, the term “heteroalkyl” should not be interpreted herein as excluding specific heteroalkyl groups, such as —CH₂O, —NR^{C}R^{D}, or the like. Each instance of a heteroalkyl group may be independently optionally substituted, i.e., unsubstituted (an “unsubstituted heteroalkyl”) or substituted (a “substituted heteroalkyl”) with one or more substituents e.g., for instance from 1 to 5 substituents, 1 to 3 substituents, or 1 substituent

As used herein, “aryl” refers to a radical of a monocyclic or polycyclic (e.g., bicyclic or tricyclic) 4n+2 aromatic ring system (e.g., having 6, 10, or 14 π electrons shared in a cyclic array) having 6-14 ring carbon atoms and zero heteroatoms provided in the aromatic ring system (“C₆-C₁₄ aryl”). In some embodiments, an aryl group has six ring carbon atoms (“C₆ aryl”; e.g., phenyl). In some embodiments, an aryl group has ten ring carbon atoms (“C₁₀ aryl”; e.g., naphthyl such as 1-naphthyl and 2-naphthyl). In some embodiments, an aryl group has fourteen ring carbon atoms (“C₁₄ aryl”; e.g., anthracyl). An aryl group may be described as, e.g., a C₆-C₁₀-membered aryl, wherein the term “membered” refers to the non-hydrogen ring atoms within the moiety. Aryl groups include phenyl, naphthyl, indenyl, and tetrahydronaphthyl. Each instance of an aryl group may be independently optionally substituted, i.e., unsubstituted (an “unsubstituted aryl”) or substituted (a “substituted aryl”) with one or more substituents. In certain embodiments, the aryl group is unsubstituted C₆-C₁₄ aryl. In certain embodiments, the aryl group is substituted C₆-C₁₄ aryl.

As used herein, “heteroaryl” refers to a radical of a 5-10 membered monocyclic or bicyclic 4n+2 aromatic ring system (e.g., having 6 or 10 π electrons shared in a cyclic array) having ring carbon atoms and 1-4 ring heteroatoms provided in the aromatic ring system, wherein each heteroatom is independently selected from nitrogen, oxygen and sulfur (“5-10 membered heteroaryl”). In heteroaryl groups that contain one or more nitrogen atoms, the point of attachment can be a carbon or nitrogen atom, as valency permits. Heteroaryl bicyclic ring systems can include one or more heteroatoms in one or both rings. “Heteroaryl” also includes ring systems wherein the heteroaryl ring, as defined above, is fused with one or more aryl groups wherein the point of attachment is either on the aryl or heteroaryl ring, and in such instances, the number of ring members designates the number of ring members in the fused (aryl/heteroaryl) ring system. Bicyclic heteroaryl groups wherein one ring does not contain a heteroatom (e.g., indolyl, quinolinyl, carbazolyl, and the like) the point of attachment can be on either ring, i.e., either the ring bearing a heteroatom (e.g., 2-indolyl) or the ring that does not contain a heteroatom (e.g., 5-indolyl). A heteroaryl group may be described as, e.g., a 6-10-membered heteroaryl, wherein the term “membered” refers to the non-hydrogen ring atoms within the moiety. Each instance of a heteroaryl group may be independently optionally substituted, i.e., unsubstituted (an “unsubstituted heteroaryl”) or substituted (a “substituted heteroaryl”) with one or more substituents e.g., for instance from 1 to 5 substituents, 1 to 3 substituents, or 1 substituent

Exemplary 5-membered heteroaryl groups containing one heteroatom include, without limitation, pyrrolyl, furanyl and thiophenyl. Exemplary 5-membered heteroaryl groups containing two heteroatoms include, without limitation, imidazolyl, pyrazolyl, oxazolyl, isoxazolyl, thiazolyl, and isothiazolyl. Exemplary 5-membered heteroaryl groups containing three heteroatoms include, without limitation, triazolyl, oxadiazolyl, and thiadiazolyl. Exemplary 5-membered heteroaryl groups containing four heteroatoms include, without limitation, tetrazolyl. Exemplary 6-membered heteroaryl groups containing one heteroatom include, without limitation, pyridinyl. Exemplary 6-membered heteroaryl groups containing two heteroatoms include, without limitation, pyridazinyl, pyrimidinyl, and pyrazinyl. Exemplary 6-membered heteroaryl groups containing three or four heteroatoms include, without limitation, triazinyl and tetrazinyl, respectively. Exemplary 7-membered heteroaryl groups containing one heteroatom include, without limitation, azepinyl, oxepinyl, and thiepinyl. Exemplary 5,6-bicyclic heteroaryl groups include, without limitation, indolyl, isoindolyl, indazolyl, benzotriazolyl, benzothiophenyl, isobenzothiophenyl, benzofuranyl, benzoisofuranyl, benzimidazolyl, benzoxazolyl, benzisoxazolyl, benzoxadiazolyl, benzthiazolyl, benzisothiazolyl, benzthiadiazolyl, indolizinyl, and purinyl. Exemplary 6,6-bicyclic heteroaryl groups include, without limitation, naphthyridinyl, pteridinyl, quinolinyl, isoquinolinyl, cinnolinyl, quinoxalinyl, phthalazinyl, and quinazolinyl. Other exemplary heteroaryl groups include heme and heme derivatives.

As used herein, “cycloalkyl” refers to a radical of a non-aromatic cyclic hydrocarbon group having from 3 to 10 ring carbon atoms (“C₃-C₁₀ cycloalkyl”) and zero heteroatoms in the non-aromatic ring system. In some embodiments, a cycloalkyl group has 3 to 8 ring carbon atoms (“C₃-C₈ cycloalkyl”). In some embodiments, a cycloalkyl group has 3 to 6 ring carbon atoms (“C₃-C₆ cycloalkyl”). In some embodiments, a cycloalkyl group has 3 to 6 ring carbon atoms (“C₃-C₆ cycloalkyl”). In some embodiments, a cycloalkyl group has 5 to 10 ring carbon atoms (“C₅-C₁₀ cycloalkyl”). A cycloalkyl group may be described as, e.g., a C₄-C₇-membered cycloalkyl, wherein the term “membered” refers to the non-hydrogen ring atoms within the moiety. Exemplary C₃-C₆ cycloalkyl groups include, without limitation, cyclopropyl (C₃), cyclopropenyl (C₃), cyclobutyl (C₄), cyclobutenyl (C₄), cyclopentyl (C₅), cyclopentenyl (C₅), cyclohexyl (C₆), cyclohexenyl (C₆), cyclohexadienyl (C₆), and the like. Exemplary C₃-C₈ cycloalkyl groups include, without limitation, the aforementioned C₃-C₆ cycloalkyl groups as well as cycloheptyl (C₇), cycloheptenyl (C₇), cycloheptadienyl (C₇), cycloheptatrienyl (C₇), cyclooctyl (C₈), cyclooctenyl (C₅), cubanyl (C₅), bicyclo[1.1.1]pentanyl (C₅), bicyclo[2.2.2]octanyl (C₅), bicyclo[2.1.1]hexanyl (C₆), bicyclo[3.1.1]heptanyl (C₇), and the like. Exemplary C₃-C₁₀ cycloalkyl groups include, without limitation, the aforementioned C₃-C₅ cycloalkyl groups as well as cyclononyl (C₉), cyclononenyl (C₉), cyclodecyl (C₁₀), cyclodecenyl (C₁₀), octahydro-1H-indenyl (C₉), decahydronaphthalenyl (C₁₀), spiro[4.5]decanyl (C₁₀), and the like. As the foregoing examples illustrate, in certain embodiments, the cycloalkyl group is either monocyclic (“monocyclic cycloalkyl”) or contain a fused, bridged or spiro ring system such as a bicyclic system (“bicyclic cycloalkyl”) and can be saturated or can be partially unsaturated. “Cycloalkyl” also includes ring systems wherein the cycloalkyl ring, as defined above, is fused with one or more aryl groups wherein the point of attachment is on the cycloalkyl ring, and in such instances, the number of carbons continue to designate the number of carbons in the cycloalkyl ring system. Each instance of a cycloalkyl group may be independently optionally substituted, i.e., unsubstituted (an “unsubstituted cycloalkyl”) or substituted (a “substituted cycloalkyl”) with one or more substituents. In certain embodiments, the cycloalkyl group is unsubstituted C₃-C₁₀ cycloalkyl. In certain embodiments, the cycloalkyl group is a substituted C₃-C₁₀ cycloalkyl.

“Heterocyclyl” as used herein refers to a radical of a 3- to 10-membered non-aromatic ring system having ring carbon atoms and 1 to 4 ring heteroatoms, wherein each heteroatom is independently selected from nitrogen, oxygen, sulfur, boron, phosphorus, and silicon (“3-10 membered heterocyclyl”). In heterocyclyl groups that contain one or more nitrogen atoms, the point of attachment can be a carbon or nitrogen atom, as valency permits. A heterocyclyl group can either be monocyclic (“monocyclic heterocyclyl”) or a fused, bridged or spiro ring system such as a bicyclic system (“bicyclic heterocyclyl”), and can be saturated or can be partially unsaturated. Heterocyclyl bicyclic ring systems can include one or more heteroatoms in one or both rings. “Heterocyclyl” also includes ring systems wherein the heterocyclyl ring, as defined above, is fused with one or more cycloalkyl groups wherein the point of attachment is either on the cycloalkyl or heterocyclyl ring, or ring systems wherein the heterocyclyl ring, as defined above, is fused with one or more aryl or heteroaryl groups, wherein the point of attachment is on the heterocyclyl ring, and in such instances, the number of ring members continue to designate the number of ring members in the heterocyclyl ring system. A heterocyclyl group may be described as, e.g., a 3-7-membered heterocyclyl, wherein the term “membered” refers to the non-hydrogen ring atoms, i.e., carbon, nitrogen, oxygen, sulfur, boron, phosphorus, and silicon, within the moiety. Each instance of heterocyclyl may be independently optionally substituted, i.e., unsubstituted (an “unsubstituted heterocyclyl”) or substituted (a “substituted heterocyclyl”) with one or more substituents. In certain embodiments, the heterocyclyl group is unsubstituted 3-10 membered heterocyclyl. In certain embodiments, the heterocyclyl group is substituted 3-10 membered heterocyclyl.

Exemplary 3-membered heterocyclyl groups containing one heteroatom include, without limitation, azirdinyl, oxiranyl, thiorenyl. Exemplary 4-membered heterocyclyl groups containing one heteroatom include, without limitation, azetidinyl, oxetanyl and thietanyl. Exemplary 5-membered heterocyclyl groups containing one heteroatom include, without limitation, tetrahydrofuranyl, dihydrofuranyl, tetrahydrothiophenyl, dihydrothiophenyl, pyrrolidinyl, dihydropyrrolyl and pyrrolyl-2,5-dione. Exemplary 5-membered heterocyclyl groups containing two heteroatoms include, without limitation, dioxolanyl, oxasulfuranyl, disulfuranyl, and oxazolidin-2-one. Exemplary 5-membered heterocyclyl groups containing three heteroatoms include, without limitation, triazolinyl, oxadiazolinyl, and thiadiazolinyl. Exemplary 6-membered heterocyclyl groups containing one heteroatom include, without limitation, piperidinyl, tetrahydropyranyl, dihydropyridinyl, and thianyl. Exemplary 6-membered heterocyclyl groups containing two heteroatoms include, without limitation, piperazinyl, morpholinyl, dithianyl, dioxanyl. Exemplary 6-membered heterocyclyl groups containing two heteroatoms include, without limitation, triazinanyl. Exemplary 7-membered heterocyclyl groups containing one heteroatom include, without limitation, azepanyl, oxepanyl and thiepanyl. Exemplary 8-membered heterocyclyl groups containing one heteroatom include, without limitation, azocanyl, oxecanyl and thiocanyl. Exemplary 5-membered heterocyclyl groups fused to a C₆ aryl ring (also referred to herein as a 5,6-bicyclic heterocyclyl ring) include, without limitation, indolinyl, isoindolinyl, dihydrobenzofuranyl, dihydrobenzothienyl, benzoxazolinonyl, and the like. Exemplary 6-membered heterocyclyl groups fused to an aryl ring (also referred to herein as a 6,6-bicyclic heterocyclyl ring) include, without limitation, tetrahydroquinolinyl, tetrahydroisoquinolinyl, and the like.

The terms “alkylene,” “alkenylene,” “alkynylene,” “haloalkylene,” “heteroalkylene,” “cycloalkylene,” or “heterocyclylene,” alone or as part of another substituent, mean, unless otherwise stated, a divalent radical derived from an alkyl, alkenyl, alkynyl, haloalkylene, heteroalkylene, cycloalkyl, or heterocyclyl respectively. For example, the term “alkenylene,” by itself or as part of another substituent, means, unless otherwise stated, a divalent radical derived from an alkene. An alkylene, alkenylene, alkynylene, haloalkylene, heteroalkylene, cycloalkylene, or heterocyclylene group may be described as, e.g., a C₁-C₆-membered alkylene, C₂-C₆-membered alkenylene, C₂-C₆-membered alkynylene, C₁-C₆-membered haloalkylene, C₁-C₆-membered heteroalkylene, C₃-C₈-membered cycloalkylene, or C₃-C₈-membered heterocyclylene, wherein the term “membered” refers to the non-hydrogen atoms within the moiety. In the case of heteroalkylene and heterocyclylene groups, heteroatoms can also occupy either or both of the chain termini (e.g., alkyleneoxy, alkylenedioxy, alkyleneamino, alkylenediamino, and the like). Still further, no orientation of the linking group is implied by the direction in which the formula of the linking group is written. For example, the formula —C(O)₂R′— may represent both —C(O)₂R′— and —R′C(O)₂—.

As used herein, the terms “cyano” or “—CN” refer to a substituent having a carbon atom joined to a nitrogen atom by a triple bond, e.g., C≡N.

As used herein, the terms “halogen” or “halo” refer to fluorine, chlorine, bromine or iodine.

As used herein, the term “hydroxy” refers to —OH.

As used herein, the term “nitro” refers to a substituent having two oxygen atoms bound to a nitrogen atom, e.g., —NO₂.

As used herein, the term “nucleobase” as used herein, is a nitrogen-containing biological compounds found linked to a sugar within a nucleoside—the basic building blocks of deoxyribonucleic acid (DNA) and ribonucleic acid (RNA). The primary, or naturally occurring, nucleobases are cytosine (DNA and RNA), guanine (DNA and RNA), adenine (DNA and RNA), thymine (DNA) and uracil (RNA), abbreviated as C, G, A, T, and U, respectively. Because A, G, C, and T appear in the DNA, these molecules are called DNA-bases; A, G, C, and U are called RNA-bases. Adenine and guanine belong to the double-ringed class of molecules called purines (abbreviated as R). Cytosine, thymine, and uracil are all pyrimidines. Other nucleobases that do not function as normal parts of the genetic code, are termed non-naturally occurring. In an embodiment, a nucleobase may be chemically modified, for example, with an alkyl (e.g., methyl), halo, —O-alkyl, or other modification.

As used herein, the term “nucleic acid” refers to deoxyribonucleic acids (DNA) or ribonucleic acids (RNA) and polymers thereof in either single- or double-stranded form. The term “nucleic acid” includes a gene, cDNA, pre-mRNA, or an mRNA. In one embodiment, the nucleic acid molecule is synthetic (e.g., chemically synthesized) or recombinant. Unless specifically limited, the term encompasses nucleic acids containing analogues or derivatives of natural nucleotides that have similar binding properties as the reference nucleic acid and are metabolized in a manner similar to naturally occurring nucleotides. Unless otherwise indicated, a particular nucleic acid sequence also implicitly encompasses conservatively modified variants thereof (e.g., degenerate codon substitutions), alleles, orthologs, SNPs, and complementarity sequences as well as the sequence explicitly indicated.

As used herein, “oxo” refers to a carbonyl, i.e., —C(O)—.

The symbol “ [symbol] ” as used herein in relation to a compound of Formula (I), (II), or (III), refers to an attachment point to another moiety or functional group within the compound.

Alkyl, alkenyl, alkynyl, haloalkyl, heteroalkyl, cycloalkyl, heterocyclyl, aryl, and heteroaryl groups, as defined herein, are optionally substituted. In general, the term “substituted”, whether preceded by the term “optionally” or not, means that at least one hydrogen present on a group (e.g., a carbon or nitrogen atom) is replaced with a permissible substituent, e.g., a substituent which upon substitution results in a stable compound, e.g., a compound which does not spontaneously undergo transformation such as by rearrangement, cyclization, elimination, or other reaction. Unless otherwise indicated, a “substituted” group has a substituent at one or more substitutable positions of the group, and when more than one position in any given structure is substituted, the substituent is either the same or different at each position. The term “substituted” is contemplated to include substitution with all permissible substituents of organic compounds, such as any of the substituents described herein that result in the formation of a stable compound. The present disclosure contemplates any and all such combinations in order to arrive at a stable compound. For purposes of this invention, heteroatoms such as nitrogen may have hydrogen substituents and/or any suitable substituent as described herein which satisfy the valencies of the heteroatoms and results in the formation of a stable moiety.

Two or more substituents may optionally be joined to form aryl, heteroaryl, cycloalkyl, or heterocyclyl groups. Such so-called ring-forming substituents are typically, though not necessarily, found attached to a cyclic base structure. In one embodiment, the ring-forming substituents are attached to adjacent members of the base structure. For example, two ring-forming substituents attached to adjacent members of a cyclic base structure create a fused ring structure. In another embodiment, the ring-forming substituents are attached to a single member of the base structure. For example, two ring-forming substituents attached to a single member of a cyclic base structure create a spirocyclic structure. In yet another embodiment, the ring-forming substituents are attached to non-adjacent members of the base structure.

Compounds described herein can comprise one or more asymmetric centers, and thus can exist in various isomeric forms, e.g., enantiomers and/or diastereomers. For example, the compounds described herein can be in the form of an individual enantiomer, diastereomer or geometric isomer, or can be in the form of a mixture of stereoisomers, including racemic mixtures and mixtures enriched in one or more stereoisomer. In an embodiment, the stereochemistry depicted in a compound is relative rather than absolute. Isomers can be isolated from mixtures by methods known to those skilled in the art, including chiral high-pressure liquid chromatography (HPLC) and the formation and crystallization of chiral salts; or preferred isomers can be prepared by asymmetric syntheses. See, for example, Jacques et al., Enantiomers, Racemates and Resolutions (Wiley Interscience, New York, 1981); Wilen et al., Tetrahedron 33:2725 (1977); Eliel, Stereochemistry of Carbon Compounds (McGraw-Hill, N Y, 1962); and Wilen, Tables of Resolving Agents and Optical Resolutions p. 268 (E. L. Eliel, Ed., Univ. of Notre Dame Press, Notre Dame, I N 1972). This disclosure additionally encompasses compounds described herein as individual isomers substantially free of other isomers, and alternatively, as mixtures of various isomers.

As used herein, a pure enantiomeric compound is substantially free from other enantiomers or stereoisomers of the compound (i.e., in enantiomeric excess). In other words, an “S” form of the compound is substantially free from the “R” form of the compound and is, thus, in enantiomeric excess of the “R” form. The term “enantiomerically pure” or “pure enantiomer” denotes that the compound comprises more than 75% by weight, more than 80% by weight, more than 85% by weight, more than 90% by weight, more than 91% by weight, more than 92% by weight, more than 93% by weight, more than 94% by weight, more than 95% by weight, more than 96% by weight, more than 97% by weight, more than 98% by weight, more than 99% by weight, more than 99.5% by weight, or more than 99.9% by weight, of the enantiomer. In certain embodiments, the weights are based upon total weight of all enantiomers or stereoisomers of the compound.

In the compositions provided herein, an enantiomerically pure compound can be present with other active or inactive ingredients. For example, a pharmaceutical composition comprising an enantiomerically pure R-compound can comprise, for example, about 90% excipient and about 10% enantiomerically pure R-compound. In certain embodiments, the enantiomerically pure R-compound in such compositions can, for example, comprise, at least about 95% by weight R-compound and at most about 5% by weight S-compound, by total weight of the compound. For example, a pharmaceutical composition comprising an enantiomerically pure S-compound can comprise, for example, about 90% excipient and about 10% enantiomerically pure S-compound. In certain embodiments, the enantiomerically pure S-compound in such compositions can, for example, comprise, at least about 95% by weight S-compound and at most about 5% by weight R-compound, by total weight of the compound.

In some embodiments, a diastereomerically pure compound can be present with other active or inactive ingredients. For example, a pharmaceutical composition comprising a diastereometerically pure exo compound can comprise, for example, about 90% excipient and about 10% diastereometerically pure exo compound. In certain embodiments, the diastereometerically pure exo compound in such compositions can, for example, comprise, at least about 95% by weight exo compound and at most about 5% by weight endo compound, by total weight of the compound. For example, a pharmaceutical composition comprising a diastereometerically pure endo compound can comprise, for example, about 90% excipient and about 10% diastereometerically pure endo compound. In certain embodiments, the diastereometerically pure endo compound in such compositions can, for example, comprise, at least about 95% by weight endo compound and at most about 5% by weight exo compound, by total weight of the compound.

In some embodiments, an isomerically pure compound can be present with other active or inactive ingredients. For example, a pharmaceutical composition comprising a isomerically pure exo compound can comprise, for example, about 90% excipient and about 10% isomerically pure exo compound. In certain embodiments, the isomerically pure exo compound in such compositions can, for example, comprise, at least about 95% by weight exo compound and at most about 5% by weight endo compound, by total weight of the compound. For example, a pharmaceutical composition comprising an isomerically pure endo compound can comprise, for example, about 90% excipient and about 10% isomerically pure endo compound. In certain embodiments, the isomerically pure endo compound in such compositions can, for example, comprise, at least about 95% by weight endo compound and at most about 5% by weight exo compound, by total weight of the compound.

In certain embodiments, the active ingredient can be formulated with little or no excipient or carrier.

Compound described herein may also comprise one or more isotopic substitutions. For example, H may be in any isotopic form, including ¹H, ²H (D or deuterium), and 3H (T or tritium); C may be in any isotopic form, including ¹¹C, ¹²C, ¹³C, and ¹⁴C; O may be in any isotopic form, including ¹⁶O and ¹⁸O; N may be in any isotopic form, including ¹⁴N and ¹⁵N; F may be in any isotopic form, including ¹⁸F, ¹⁹F, and the like.

The term “pharmaceutically acceptable salt” is meant to include salts of the active compounds that are prepared with relatively nontoxic acids or bases, depending on the particular substituents found on the compounds described herein. When compounds of the present disclosure contain relatively acidic functionalities, base addition salts can be obtained by contacting the neutral form of such compounds with a sufficient amount of the desired base, either neat or in a suitable inert solvent. Examples of pharmaceutically acceptable base addition salts include sodium, potassium, calcium, ammonium, organic amino, or magnesium salt, or a similar salt. When compounds of the present invention contain relatively basic functionalities, acid addition salts can be obtained by contacting the neutral form of such compounds with a sufficient amount of the desired acid, either neat or in a suitable inert solvent. Examples of pharmaceutically acceptable acid addition salts include those derived from inorganic acids like hydrochloric, hydrobromic, nitric, carbonic, monohydrogencarbonic, phosphoric, monohydrogenphosphoric, dihydrogenphosphoric, sulfuric, monohydrogensulfuric, hydriodic, or phosphorous acids and the like, as well as the salts derived from organic acids like acetic, propionic, isobutyric, maleic, malonic, benzoic, succinic, suberic, fumaric, lactic, mandelic, phthalic, benzenesulfonic, p-tolylsulfonic, citric, tartaric, methanesulfonic, and the like. Also included are salts of amino acids such as arginate and the like, and salts of organic acids like glucuronic or galactunoric acids and the like (see, e.g., Berge et al, Journal of Pharmaceutical Science 66: 1-19 (1977)). Certain specific compounds of the present invention contain both basic and acidic functionalities that allow the compounds to be converted into either base or acid addition salts. These salts may be prepared by methods known to those skilled in the art. Other pharmaceutically acceptable carriers known to those of skill in the art are suitable for the present invention.

In addition to salt forms, the present disclosure provides compounds in a prodrug form. Prodrugs of the compounds described herein are those compounds that readily undergo chemical changes under physiological conditions to provide the compounds of the present invention. Additionally, prodrugs can be converted to the compounds of the present invention by chemical or biochemical methods in an ex vivo environment. For example, prodrugs can be slowly converted to the compounds of the present invention when placed in a transdermal patch reservoir with a suitable enzyme or chemical reagent.

The term “solvate” refers to forms of the compound that are associated with a solvent, usually by a solvolysis reaction. This physical association may include hydrogen bonding. Conventional solvents include water, methanol, ethanol, acetic acid, DMSO, THF, diethyl ether, and the like. The compounds of Formula (I), (II), (III), or (IV), may be prepared, e.g., in crystalline form, and may be solvated. Suitable solvates include pharmaceutically acceptable solvates and further include both stoichiometric solvates and non-stoichiometric solvates. In certain instances, the solvate will be capable of isolation, for example, when one or more solvent molecules are incorporated in the crystal lattice of a crystalline solid. “Solvate” encompasses both solution-phase and isolable solvates. Representative solvates include hydrates, ethanolates, and methanolates.

The term “hydrate” refers to a compound which is associated with water. Typically, the number of the water molecules contained in a hydrate of a compound is in a definite ratio to the number of the compound molecules in the hydrate. Therefore, a hydrate of a compound may be represented, for example, by the general formula R·x H₂O, wherein R is the compound and wherein x is a number greater than 0. A given compound may form more than one type of hydrates, including, e.g., monohydrates (x is 1), lower hydrates (x is a number greater than 0 and smaller than 1, e.g., hemihydrates (R·0.5 H₂O)), and polyhydrates (x is a number greater than 1, e.g., dihydrates (R·2 H₂O) and hexahydrates (R·6 H₂O)).

The term “tautomer” refers to compounds that are interchangeable forms of a particular compound structure, and that vary in the displacement of hydrogen atoms and electrons. Thus, two structures may be in equilibrium through the movement of π electrons and an atom (usually H). For example, enols and ketones are tautomers because they are rapidly interconverted by treatment with either acid or base. Another example of tautomerism is the aci- and nitro-forms of phenylnitromethane that are likewise formed by treatment with acid or base. Tautomeric forms may be relevant to the attainment of the optimal chemical reactivity and biological activity of a compound of interest.

### Other Definitions

The following definitions are more general terms used throughout the present disclosure.

The articles “a” and “an” refer to one or more than one (e.g., to at least one) of the grammatical object of the article. By way of example, “an element” means one element or more than one element. The term “and/or” means either “and” or “or” unless indicated otherwise.

The term “about” is used herein to mean within the typical ranges of tolerances in the art. For example, “about” can be understood as about 2 standard deviations from the mean. In certain embodiments, about means ±10%. In certain embodiments, about means±5%. When about is present before a series of numbers or a range, it is understood that “about” can modify each of the numbers in the series or range.

“Acquire” or “acquiring” as used herein, refer to obtaining possession of a value, e.g., a numerical value, or image, or a physical entity (e.g., a sample), by “directly acquiring” or “indirectly acquiring” the value or physical entity. “Directly acquiring” means performing a process (e.g., performing an analytical method or protocol) to obtain the value or physical entity. “Indirectly acquiring” refers to receiving the value or physical entity from another party or source (e.g., a third-party laboratory that directly acquired the physical entity or value). Directly acquiring a value or physical entity includes performing a process that includes a physical change in a physical substance or the use of a machine or device. Examples of directly acquiring a value include obtaining a sample from a human subject. Directly acquiring a value includes performing a process that uses a machine or device, e.g., mass spectrometer to acquire mass spectrometry data.

The terms “administer,” “administering,” or “administration,” as used herein refers to implanting, absorbing, ingesting, injecting, inhaling, or otherwise introducing an inventive compound, or a pharmaceutical composition thereof.

As used herein, the terms “condition,” “disease,” and “disorder” are used interchangeably.

An “effective amount” of a compound of Formula (I), (II), (III), or (IV) refers to an amount sufficient to elicit the desired biological response, i.e., treating the condition. As will be appreciated by those of ordinary skill in this art, the effective amount of a compound of Formula (I), (II), (III), or (IV) may vary depending on such factors as the desired biological endpoint, the pharmacokinetics of the compound, the condition being treated, the mode of administration, and the age and health of the subject. An effective amount encompasses therapeutic and prophylactic treatment. For example, in treating cancer, an effective amount of an inventive compound may reduce the tumor burden or stop the growth or spread of a tumor.

A “therapeutically effective amount” of a compound of Formula (I), (II), (III), or (IV) is an amount sufficient to provide a therapeutic benefit in the treatment of a condition or to delay or minimize one or more symptoms associated with the condition. In some embodiments, a therapeutically effective amount is an amount sufficient to provide a therapeutic benefit in the treatment of a condition or to minimize one or more symptoms associated with the condition. A therapeutically effective amount of a compound means an amount of therapeutic agent, alone or in combination with other therapies, which provides a therapeutic benefit in the treatment of the condition. The term “therapeutically effective amount” can encompass an amount that improves overall therapy, reduces or avoids symptoms or causes of the condition, or enhances the therapeutic efficacy of another therapeutic agent.

The terms “peptide,” “polypeptide,” and “protein” are used interchangeably, and refer to a compound comprised of amino acid residues covalently linked by peptide bonds. A protein or peptide must contain at least two amino acids, and no limitation is placed on the maximum number of amino acids that can comprised therein. Polypeptides include any peptide or protein comprising two or more amino acids joined to each other by peptide bonds. As used herein, the term refers to both short chains, which also commonly are referred to in the art as peptides, oligopeptides and oligomers, for example, and to longer chains, which generally are referred to in the art as proteins, of which there are many types.

“Prevention,” “prevent,” and “preventing” as used herein refers to a treatment that comprises administering a therapy, e.g., administering a compound described herein (e.g., a compound of Formula (I), (II), (III), or (IV)) prior to the onset of a disease, disorder, or condition in order to preclude the physical manifestation of said disease, disorder, or condition. In some embodiments, “prevention,” “prevent,” and “preventing” require that signs or symptoms of the disease, disorder, or condition have not yet developed or have not yet been observed. In some embodiments, treatment comprises prevention and in other embodiments it does not.

A “subject” to which administration is contemplated includes, but is not limited to, humans (i.e., a male or female of any age group, e.g., a pediatric subject (e.g., infant, child, adolescent) or adult subject (e.g., young adult, middle-aged adult, or senior adult)) and/or other non-human animals, for example, mammals (e.g., primates (e.g., cynomolgus monkeys, rhesus monkeys); commercially relevant mammals such as cattle, pigs, horses, sheep, goats, cats, and/or dogs) and birds (e.g., commercially relevant birds such as chickens, ducks, geese, and/or turkeys). In certain embodiments, the animal is a mammal. The animal may be a male or female and at any stage of development. A non-human animal may be a transgenic animal.

As used herein, the terms “treatment,” “treat,” and “treating” refer to reversing, alleviating, delaying the onset of, or inhibiting the progress of one or more of a symptom, manifestation, or underlying cause of a disease, disorder, or condition (e.g., as described herein), e.g., by administering a therapy, e.g., administering a compound described herein (e.g., a compound of Formula (I), (II), (III), or (IV)). In an embodiment, treating comprises reducing, reversing, alleviating, delaying the onset of, or inhibiting the progress of a symptom of a disease, disorder, or condition. In an embodiment, treating comprises reducing, reversing, alleviating, delaying the onset of, or inhibiting the progress of a manifestation of a disease, disorder, or condition. In an embodiment, treating comprises reducing, reversing, alleviating, reducing, or delaying the onset of, an underlying cause of a disease, disorder, or condition. In some embodiments, “treatment,” “treat,” and “treating” require that signs or symptoms of the disease, disorder, or condition have developed or have been observed. In other embodiments, treatment may be administered in the absence of signs or symptoms of the disease or condition, e.g., in preventive treatment. For example, treatment may be administered to a susceptible individual prior to the onset of symptoms (e.g., in light of a history of symptoms and/or in light of genetic or other susceptibility factors). Treatment may also be continued after symptoms have resolved, for example, to delay or prevent recurrence. Treatment may also be continued after symptoms have resolved, for example, to delay or prevent recurrence. In some embodiments, treatment comprises prevention and in other embodiments it does not.

A “proliferative disease” refers to a disease that occurs due to abnormal extension by the multiplication of cells (Walker, Cambridge Dictionary of Biology; Cambridge University Press: Cambridge, UK, 1990). A proliferative disease may be associated with: 1) the pathological proliferation of normally quiescent cells; 2) the pathological migration of cells from their normal location (e.g., metastasis of neoplastic cells); 3) the pathological expression of proteolytic enzymes such as the matrix metalloproteinases (e.g., collagenases, gelatinases, and elastases); 4) the pathological angiogenesis as in proliferative retinopathy and tumor metastasis; or 5) evasion of host immune surveillance and elimination of neoplastic cells. Exemplary proliferative diseases include cancers (i.e., “malignant neoplasms”), benign neoplasms, and angiogenesis.

A “non-proliferative disease” refers to a disease that does not primarily extend through the abnormal multiplication of cells. A non-proliferative disease may be associated with any cell type or tissue type in a subject. Exemplary non-proliferative diseases include neurological diseases or disorders (e.g., a repeat expansion disease); autoimmune disease or disorders; immunodeficiency diseases or disorders; lysosomal storage diseases or disorders; inflammatory diseases or disorders; cardiovascular conditions, diseases, or disorders; metabolic diseases or disorders; respiratory conditions, diseases, or disorders; renal diseases or disorders; and infectious diseases.

### Compounds

In one aspect, the present disclosure features a compound of Formula (I):

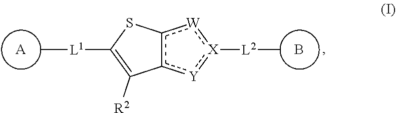

or a pharmaceutically acceptable salt, solvate, hydrate, tautomer, or stereoisomer thereof, wherein A and B are each independently cycloalkyl, heterocyclyl, aryl, or heteroaryl, each of which is optionally substituted with one or more R¹; L¹ is absent, C₁-C₆-alkylene, C₁-C₆-heteroalkylene, —O—, —C(O)—, —N(R³)—, —N(R³)C(O)—, or —C(O)N(R³)—, wherein each alkylene and heteroalkylene is optionally substituted with one or more R⁴; L² is absent, C₁-C₆-alkylene, C₁-C₆-heteroalkylene, C₆-C₁₂-arylene, C₅-C₁₂-heteroarylene, —O—, —C(O)—, —N(R³)—, —N(R³)C(O)—, or —C(O)N(R³)—, wherein each alkylene, heteroalkylene, arylene, and heteroarylene is optionally substituted with one or more R⁴; W and Y are each independently C, C(R⁵) or N; X is C or N; wherein at least one of W, X, and Y is N, and the dashed lines in the ring comprising W, X, and Y may be single or double bonds as valency permits; each R¹ is independently hydrogen, C₁-C₆-alkyl, C₂-C₆-alkenyl, C₂-C₆-alkynyl, C₁-C₆-heteroalkyl, C₁-C₆-haloalkyl, cycloalkyl, heterocyclyl, aryl, C₁-C₆ alkylene-cycloalkyl, C₁-C₆ alkylene-heterocyclyl, C₁-C₆ alkylene-aryl, C₁-C₆ alkenylene-aryl, C₁-C₆ alkylene-heteroaryl, heteroaryl, halo, cyano, oxo, —OR^{A}, —NR^{B}R^{C}, —NR^{B}C(O)R^{D}, —NO₂, —C(O)NR^{B}R^{C}, —C(O)R^{D}, —C(O)OR^{D}, or —S(O)ₓR^{D}, wherein each alkyl, alkylene, alkenyl, alkynyl, heteroalkyl, haloalkyl, cycloalkyl, heterocyclyl, aryl, and heteroaryl is optionally substituted with one or more R⁶; or two R¹ groups, together with the atoms to which they are attached, form a 3-7-membered cycloalkyl, heterocyclyl, aryl, or heteroaryl, wherein each cycloalkyl, heterocyclyl, aryl, and heteroaryl is optionally substituted with one or more R⁶; R² is hydrogen, halo, cyano, C₁-C₆-alkyl, C₂-C₆-alkenyl, C₂-C₆-alkynyl, C₁-C₆-heteroalkyl, C₁-C₆-haloalkyl, or —OR^{A}; each R³ is independently hydrogen, C₁-C₆-alkyl, or C₁-C₆-haloalkyl; each R⁴ is independently C₁-C₆-alkyl, C₁-C₆-heteroalkyl, C₁-C₆-haloalkyl, cycloalkyl, halo, cyano, oxo, —OR^{A}, —NRBR^{C}, —C(O)R^{D}, or —C(O)OR^{D}; R⁵ is hydrogen, C₁-C₆-alkyl, C₂-C₆-alkenyl, C₂-C₆-alkynyl, C₁-C₆-heteroalkyl, C₁-C₆-haloalkyl, halo, cyano, or —OR^{A}; each R⁶ is independently C₁-C₆-alkyl, C₂-C₆-alkenyl, C₂-C₆-alkynyl, C₁-C₆-heteroalkyl, C₁-C₆-haloalkyl, cycloalkyl, heterocyclyl, aryl, heteroaryl, halo, cyano, oxo, —OR^{A}, —NR^{B}R^{C}, —NR^{B}C(O)R^{D}, —NO₂, —C(O)NR^{B}R^{C}, —C(O)R^{D}, —C(O)OR^{D}, or —S(O)ₓR^{D}, wherein each of alkyl, alkenyl, alkynyl, heteroalkyl, haloalkyl, cycloalkyl, heterocyclyl, aryl, and heteroaryl is optionally substituted with one or more R⁷; each R^{A} is independently hydrogen, C₁-C₆ alkyl, C₁-C₆ heteroalkyl, C₁-C₆ haloalkyl, cycloalkyl, heterocyclyl, aryl, heteroaryl, C₁-C₆ alkylene-aryl, C₁-C₆ alkylene-heteroaryl, —C(O)R^{D}, or —S(O)ₓR^{D}, wherein each alkyl, alkylene, heteroalkyl, haloalkyl, cycloalkyl, heterocyclyl, aryl, and heteroaryl is optionally substituted with one or more R⁸; each R^{B} and R^{C} is independently hydrogen, C₁-C₆ alkyl, C₁-C₆ heteroalkyl, C₁-C₆ haloalkyl, cycloalkyl, heterocyclyl, aryl, heteroaryl, C₁-C₆ alkylene-cycloalkyl, C₁-C₆ alkylene-heterocyclyl, —OR^{A}, wherein each alkyl, alkylene, heteroalkyl, haloalkyl, cycloalkyl, heterocyclyl, aryl, and heteroaryl is optionally substituted with one or more R⁸; or R^{B} and R^{C} together with the atom to which they are attached form a 3-7-membered heterocyclyl ring optionally substituted with one or more R⁸; each R^{D} is independently hydrogen, C₁-C₆ alkyl, C₂-C₆ alkenyl, C₂-C₆ alkynyl, C₁-C₆ heteroalkyl, C₁-C₆ haloalkyl, wherein each alkyl, alkenyl, alkynyl, heteroalkyl, and haloalkyl is optionally substituted with one or more R⁸; each R⁷ is independently C₁-C₆ alkyl, C₁-C₆ heteroalkyl, C₁-C₆ haloalkyl, cycloalkyl, heterocyclyl, aryl, heteroaryl, halo, cyano, oxo, or —OR^{A}; each R⁸ is C₁-C₆-alkyl, halo, cyano, oxo, or —OR^{A1}; each R^{A1} is hydrogen or C₁-C₆-alkyl; and x is 0, 1, or 2.

In another aspect, the present invention features a compound of Formula (II):

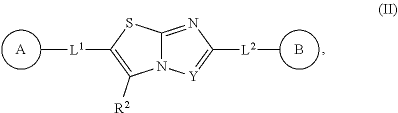

or a pharmaceutically acceptable salt, solvate, hydrate, tautomer, or stereoisomer thereof, wherein A and B are each independently cycloalkyl, heterocyclyl, aryl, or heteroaryl, each of which is optionally substituted with one or more R¹; L¹ is absent, C₁-C₆-alkylene, C₁-C₆-heteroalkylene, —O—, —C(O)—, —N(R³)—, —N(R³)C(O)—, or —C(O)N(R³)—, wherein each alkylene and heteroalkylene is optionally substituted with one or more R⁴; L² is absent, C₁-C₆-alkylene, C₁-C₆-heteroalkylene, C₆-C₁₂-arylene, C₅-C₁₂-heteroarylene, —O—, —C(O)—, —N(R³)—, —N(R³)C(O)—, or —C(O)N(R³)—, wherein each alkylene, heteroalkylene, arylene, and heteroarylene is optionally substituted with one or more R⁴; Y is C(R⁵) or N; each R¹ is independently hydrogen, C₁-C₆-alkyl, C₂-C₆-alkenyl, C₂-C₆-alkynyl, C₁-C₆-heteroalkyl, C₁-C₆-haloalkyl, cycloalkyl, heterocyclyl, aryl, C₁-C₆ alkylene-cycloalkyl, C₁-C₆ alkylene-heterocyclyl, C₁-C₆ alkylene-aryl, C₁-C₆ alkenylene-aryl, C₁-C₆ alkylene-heteroaryl, heteroaryl, halo, cyano, oxo, —OR^{A}, —NR^{B}R^{C}, —NR^{B}C(O)R^{D}, —NO₂, —C(O)NR^{B}R^{C}, —C(O)R^{D}, —C(O)OR^{D}, or —S(O)ₓR^{D}, wherein each alkyl, alkylene, alkenyl, alkynyl, heteroalkyl, haloalkyl, cycloalkyl, heterocyclyl, aryl, and heteroaryl is optionally substituted with one or more R⁶; or two R¹ groups, together with the atoms to which they are attached, form a 3-7-membered cycloalkyl, heterocyclyl, aryl, or heteroaryl, wherein each cycloalkyl, heterocyclyl, aryl, and heteroaryl is optionally substituted with one or more R⁶; R² is hydrogen, halo, cyano, C₁-C₆-alkyl, C₂-C₆-alkenyl, C₂-C₆-alkynyl, C₁-C₆-heteroalkyl, or C₁-C₆-haloalkyl; each R³ is independently hydrogen, C₁-C₆-alkyl, or C₁-C₆-haloalkyl; each R⁴ is independently C₁-C₆-alkyl, C₁-C₆-heteroalkyl, C₁-C₆-haloalkyl, cycloalkyl, halo, cyano, oxo, —OR^{A}, —NR^{B}R^{C}, —C(O)R^{D}, or —C(O)OR^{D}; R⁵ is hydrogen or C₁-C₆-alkyl, C₂-C₆-alkenyl, C₂-C₆-alkynyl, C₁-C₆-heteroalkyl, C₁-C₆-haloalkyl, halo, cyano, or —OR^{A}; each R⁶ is independently C₁-C₆-alkyl, C₂-C₆-alkenyl, C₂-C₆-alkynyl, C₁-C₆-heteroalkyl, C₁-C₆-haloalkyl, cycloalkyl, heterocyclyl, aryl, heteroaryl, halo, cyano, oxo, —OR^{A}, —NR^{B}R^{C}, —NR^{B}C(O)R^{D}, —NO₂, —C(O)NR^{B}R^{C}, —C(O)R^{D}, —C(O)OR^{D}, or —S(O)ₓR^{D}, wherein each of alkyl, alkenyl, alkynyl, heteroalkyl, haloalkyl, cycloalkyl, heterocyclyl, aryl, and heteroaryl is optionally substituted with one or more R⁷; R^{A} is independently hydrogen, C₁-C₆ alkyl, C₁-C₆ heteroalkyl, C₁-C₆ haloalkyl, cycloalkyl, heterocyclyl, aryl, heteroaryl, C₁-C₆ alkylene-aryl, C₁-C₆ alkylene-heteroaryl, —C(O)R^{D}, or —S(O)ₓR^{D}, wherein each alkyl, alkylene, heteroalkyl, haloalkyl, cycloalkyl, heterocyclyl, aryl, and heteroaryl is optionally substituted with one or more R⁸; each R^{B} and R^{C} is independently hydrogen, C₁-C₆ alkyl, C₁-C₆ heteroalkyl, C₁-C₆ haloalkyl, cycloalkyl, heterocyclyl, aryl, heteroaryl, C₁-C₆ alkylene-cycloalkyl, C₁-C₆ alkylene-heterocyclyl, —OR^{A}, wherein each alkyl, alkylene, heteroalkyl, haloalkyl, cycloalkyl, heterocyclyl, aryl, and heteroaryl is optionally substituted with one or more R⁸; or R^{B} and R^{C} together with the atom to which they are attached form a 3-7-membered heterocyclyl ring optionally substituted with one or more R⁸; each R^{D} is independently hydrogen, C₁-C₆ alkyl, C₂-C₆ alkenyl, C₂-C₆ alkynyl, C₁-C₆ heteroalkyl, C₁-C₆ haloalkyl, wherein each alkyl, alkenyl, alkynyl, heteroalkyl, and haloalkyl is optionally substituted with one or more R⁸; each R⁷ is independently C₁-C₆ alkyl, C₁-C₆ heteroalkyl, C₁-C₆ haloalkyl, cycloalkyl, heterocyclyl, aryl, heteroaryl, halo, cyano, oxo, or —OR^{A}; each R⁸ is C₁-C₆-alkyl, halo, cyano, oxo, or —OR^{A1}; each R^{A1} is hydrogen or C₁-C₆-alkyl; and x is 0, 1, or 2.

In another aspect, the present disclosure features a compound of Formula (III):

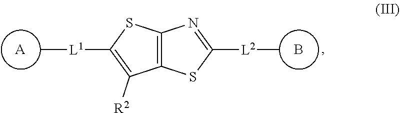

or a pharmaceutically acceptable salt, solvate, hydrate, tautomer, or stereoisomer thereof, wherein A and B are each independently cycloalkyl, heterocyclyl, aryl, or heteroaryl, each of which is optionally substituted with one or more R¹; L¹ is absent, C₁-C₆-alkylene, C₁-C₆-heteroalkylene, —O—, —C(O)—, —N(R³)—, —N(R³)C(O)—, or —C(O)N(R³)—, wherein each alkylene and heteroalkylene is optionally substituted with one or more R⁴; L² is absent, C₁-C₆-alkylene, C₁-C₆-heteroalkylene, C₆-C₁₂-arylene, C₅-C₁₂-heteroarylene, —O—, —C(O)—, —N(R³)—, —N(R³)C(O)—, or —C(O)N(R³)—, wherein each alkylene, heteroalkylene, arylene, and heteroarylene is optionally substituted with one or more R⁴; each R¹ is independently hydrogen, C₁-C₆-alkyl, C₂-C₆-alkenyl, C₂-C₆-alkynyl, C₁-C₆-heteroalkyl, C₁-C₆-haloalkyl, cycloalkyl, heterocyclyl, aryl, C₁-C₆ alkylene-cycloalkyl, C₁-C₆ alkylene-heterocyclyl, C₁-C₆ alkylene-aryl, C₁-C₆ alkenylene-aryl, C₁-C₆ alkylene-heteroaryl, heteroaryl, halo, cyano, oxo, —ORA, —NRBRc, —N^{B}C(O)R^{D} NO₂, —C(O)NR^{B}R^{C}, —C(O)R^{D}, —C(O)OR^{D}, or —S(O)ₓR^{D}, wherein each alkyl, alkylene, alkenyl, alkynyl, heteroalkyl, haloalkyl, cycloalkyl, heterocyclyl, aryl, and heteroaryl is optionally substituted with one or more R⁶; or two R¹ groups, together with the atoms to which they are attached, form a 3-7-membered cycloalkyl, heterocyclyl, aryl, or heteroaryl, wherein each cycloalkyl, heterocyclyl, aryl, and heteroaryl is optionally substituted with one or more R⁶; R² is hydrogen, halo, cyano, C₁-C₆-alkyl, C₂-C₆-alkenyl, C₂-C₆-alkynyl, C₁-C₆-heteroalkyl, or C₁-C₆-haloalkyl; each R³ is independently hydrogen, C₁-C₆-alkyl, or C₁-C₆-haloalkyl; each R⁴ is independently C₁-C₆-alkyl, C₁-C₆-heteroalkyl, C₁-C₆-haloalkyl, cycloalkyl, halo, cyano, oxo, —OR^{A}, —NR^{B}R^{C}, —C(O)R^{D}, or —C(O)OR^{D}; each R⁶ is independently C₁-C₆-alkyl, C₂-C₆-alkenyl, C₂-C₆-alkynyl, C₁-C₆-heteroalkyl, C₁-C₆-haloalkyl, cycloalkyl, heterocyclyl, aryl, heteroaryl, halo, cyano, oxo, —OR^{A}, —NR^{B}R^{C}, —N^{B}C(O)R^{D}, —NO₂, —C(O)NR^{B}R^{C}, —C(O)R^{D}, —C(O)OR^{D}, or —S(O)ₓR^{D}, wherein each of alkyl, alkenyl, alkynyl, heteroalkyl, haloalkyl, cycloalkyl, heterocyclyl, aryl, and heteroaryl is optionally substituted with one or more R⁷; R^{A} is independently hydrogen, C₁-C₆ alkyl, C₁-C₆ heteroalkyl, C₁-C₆ haloalkyl, cycloalkyl, heterocyclyl, aryl, heteroaryl, C₁-C₆ alkylene-aryl, C₁-C₆ alkylene-heteroaryl, —C(O)R^{D}, or —S(O)ₓR^{D}, wherein each alkyl, alkylene, heteroalkyl, haloalkyl, cycloalkyl, heterocyclyl, aryl, and heteroaryl is optionally substituted with one or more R⁸; each R^{B} and R^{C} is independently hydrogen, C₁-C₆ alkyl, C₁-C₆ heteroalkyl, C₁-C₆ haloalkyl, cycloalkyl, heterocyclyl, aryl, heteroaryl, C₁-C₆ alkylene-cycloalkyl, C₁-C₆ alkylene-heterocyclyl, —OR^{A}, wherein each alkyl, alkylene, heteroalkyl, haloalkyl, cycloalkyl, heterocyclyl, aryl, and heteroaryl is optionally substituted with one or more R⁸; or R^{B} and R^{C} together with the atom to which they are attached form a 3-7-membered heterocyclyl ring optionally substituted with one or more R⁸; each R^{D} is independently hydrogen, C₁-C₆ alkyl, C₂-C₆ alkenyl, C₂-C₆ alkynyl, C₁-C₆ heteroalkyl, C₁-C₆ haloalkyl, wherein each alkyl, alkenyl, alkynyl, heteroalkyl, and haloalkyl is optionally substituted with one or more R⁸; each R⁷ is independently C₁-C₆ alkyl, C₁-C₆ heteroalkyl, C₁-C₆ haloalkyl, cycloalkyl, heterocyclyl, aryl, heteroaryl, halo, cyano, oxo, or —OR^{A}; each R⁸ is C₁-C₆-alkyl, halo, cyano, oxo, or —OR^{A1}; each R^{A1} is hydrogen or C₁-C₆-alkyl; and x is 0, 1, or 2.

In another aspect, the present disclosure features a compound of Formula (IV):

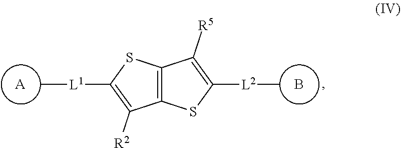

or a pharmaceutically acceptable salt, solvate, hydrate, tautomer, or stereoisomer thereof, wherein A and B are each independently cycloalkyl, heterocyclyl, aryl, or heteroaryl, each of which is optionally substituted with one or more R¹; L¹ is absent, C₁-C₆-alkylene, C₁-C₆-heteroalkylene, —O—, —C(O)—, —N(R³)—, —N(R³)C(O)—, or —C(O)N(R³)—, wherein each alkylene and heteroalkylene is optionally substituted with one or more R⁴; L² is absent, C₁-C₆-alkylene, C₁-C₆-heteroalkylene, C₆-C₁₂-arylene, C₅-C₁₂-heteroarylene, —O—, —C(O)—, —N(R³)—, —N(R³)C(O)—, or —C(O)N(R³)—, wherein each alkylene, heteroalkylene, arylene, and heteroarylene is optionally substituted with one or more R⁴; each R¹ is independently hydrogen, C₁-C₆-alkyl, C₂-C₆-alkenyl, C₂-C₆-alkynyl, C₁-C₆-heteroalkyl, C₁-C₆-haloalkyl, cycloalkyl, heterocyclyl, aryl, C₁-C₆ alkylene-cycloalkyl, C₁-C₆ alkylene-heterocyclyl, C₁-C₆ alkylene-aryl, C₁-C₆ alkenylene-aryl, C₁-C₆ alkylene-heteroaryl, heteroaryl, halo, cyano, oxo, —OR^{A}, —NR^{B}R^{C}, —NR^{B}C(O)R^{D} NO₂, —C(O)NR^{B}R^{C}, —C(O)R^{D}, —C(O)OR^{D}, or —S(O)ₓR^{D}, wherein each alkyl, alkylene, alkenyl, alkynyl, heteroalkyl, haloalkyl, cycloalkyl, heterocyclyl, aryl, and heteroaryl is optionally substituted with one or more R⁶; or two R¹ groups, together with the atoms to which they are attached, form a 3-7-membered cycloalkyl, heterocyclyl, aryl, or heteroaryl, wherein each cycloalkyl, heterocyclyl, aryl, and heteroaryl is optionally substituted with one or more R⁶; R² and R⁵ are each independently hydrogen, halo, cyano, C₁-C₆-alkyl, C₂-C₆-alkenyl, C₂-C₆-alkynyl, C₁-C₆-heteroalkyl, or C₁-C₆-haloalkyl; each R³ is independently hydrogen, C₁-C₆-alkyl, or C₁-C₆-haloalkyl; each R⁴ is independently C₁-C₆-alkyl, C₁-C₆-heteroalkyl, C₁-C₆-haloalkyl, cycloalkyl, halo, cyano, oxo, —OR^{A}, —NR^{B}R^{C}, —C(O)R^{D}, or —C(O)OR^{D}; each R⁶ is independently C₁-C₆-alkyl, C₂-C₆-alkenyl, C₂-C₆-alkynyl, C₁-C₆-heteroalkyl, C₁-C₆-haloalkyl, cycloalkyl, heterocyclyl, aryl, heteroaryl, halo, cyano, oxo, —OR^{A}, —NR^{B}R^{C}, NR^{B}C(O)R^{D}, —NO₂, —C(O)NR^{B}R^{C}, —C(O)R^{D}, —C(O)OR^{D}, or —S(O)ₓR^{D}, wherein each of alkyl, alkenyl, alkynyl, heteroalkyl, haloalkyl, cycloalkyl, heterocyclyl, aryl, and heteroaryl is optionally substituted with one or more R⁷; R^{A} is independently hydrogen, C₁-C₆ alkyl, C₁-C₆ heteroalkyl, C₁-C₆ haloalkyl, cycloalkyl, heterocyclyl, aryl, heteroaryl, C₁-C₆ alkylene-aryl, C₁-C₆ alkylene-heteroaryl, —C(O)R^{D}, or —S(O)ₓR^{D}, wherein each alkyl, alkylene, heteroalkyl, haloalkyl, cycloalkyl, heterocyclyl, aryl, and heteroaryl is optionally substituted with one or more R⁸; each R^{B} and R^{C} is independently hydrogen, C₁-C₆ alkyl, C₁-C₆ heteroalkyl, C₁-C₆ haloalkyl, cycloalkyl, heterocyclyl, aryl, heteroaryl, C₁-C₆ alkylene-cycloalkyl, C₁-C₆ alkylene-heterocyclyl, —OR^{A}, wherein each alkyl, alkylene, heteroalkyl, haloalkyl, cycloalkyl, heterocyclyl, aryl, and heteroaryl is optionally substituted with one or more R⁸; or R^{B} and R^{C} together with the atom to which they are attached form a 3-7-membered heterocyclyl ring optionally substituted with one or more R⁸; each R^{D} is independently hydrogen, C₁-C₆ alkyl, C₂-C₆ alkenyl, C₂-C₆ alkynyl, C₁-C₆ heteroalkyl, C₁-C₆ haloalkyl, wherein each alkyl, alkenyl, alkynyl, heteroalkyl, and haloalkyl is optionally substituted with one or more R⁸; each R⁷ is independently C₁-C₆ alkyl, C₁-C₆ heteroalkyl, C₁-C₆ haloalkyl, cycloalkyl, heterocyclyl, aryl, heteroaryl, halo, cyano, oxo, or —OR^{A}; each R⁸ is C₁-C₆-alkyl, halo, cyano, oxo, or —OR^{A1}; each R^{A1} is hydrogen or C₁-C₆-alkyl; and x is 0, 1, or 2.

As generally described herein for compounds of Formula (I), (II), (III), and (IV), each of A or B are independently cycloalkyl, heterocyclyl, aryl, or heteroaryl, each of which is optionally substituted with one or more R¹.

For each of Formulas (I), (II), (III), or (IV), in some embodiments, A and B are independently a monocyclic ring, e.g., monocyclic cycloalkyl, monocyclic heterocyclyl, monocyclic aryl, or monocyclic heteroaryl. The monocyclic ring may be saturated, partially unsaturated, or fully unsaturated (e.g., aromatic). In some embodiments, A or B are independently a monocyclic ring comprising between 3 and 10 ring atoms (e.g., 3, 4, 5, 6, 7, 8, 9, or 10 ring atoms). In some embodiments, A is a 4-membered monocyclic ring. In some embodiments, B is a 4-membered monocyclic ring. In some embodiments, A is a 5-membered monocyclic ring. In some embodiments, B is a 5-membered monocyclic ring. In some embodiments, A is a 6-membered monocyclic ring. In some embodiments, B is a 6-membered monocyclic ring. In some embodiments, A is a 7-membered monocyclic ring. In some embodiments, B is a 7-membered monocyclic ring. In some embodiments, A is an 8-membered monocyclic ring. In some embodiments, B is an 8-membered monocyclic ring. In some embodiments, A or B are independently a monocyclic ring optionally substituted with one or more R¹.

In some embodiments, A and B are independently a bicyclic ring, e.g., bicyclic cycloalkyl, bicyclic heterocyclyl, bicyclic aryl, or bicyclic heteroaryl. The bicyclic ring may be saturated, partially unsaturated, or fully unsaturated (e.g., aromatic). In some embodiments, A or B are independently a bicyclic ring comprising a fused, bridged, or spiro ring system. In some embodiments, A or B are independently a bicyclic ring comprising between 4 and 18 ring atoms (e.g., 4, 5, 6, 7, 8, 9, 10, 11, 12, 13, 14, 15, 16, 17, or 18 ring atoms). In some embodiments, A is a 6-membered bicyclic ring. In some embodiments, B is a 6-membered bicyclic ring. In some embodiments, A is a 7-membered bicyclic ring. In some embodiments, B is a 7-membered bicyclic ring. In some embodiments, A is an 8-membered bicyclic ring. In some embodiments, B is an 8-membered bicyclic ring. In some embodiments, A is a 9-membered bicyclic ring. In some embodiments, B is a 9-membered bicyclic ring. In some embodiments, A is a 10-membered bicyclic ring. In some embodiments, B is a 10-membered bicyclic ring. In some embodiments, A is an 11-membered bicyclic ring. In some embodiments, B is an 11-membered bicyclic ring. In some embodiments, A is a 12-membered bicyclic ring. In some embodiments, B is a 12-membered bicyclic ring. In some embodiments, A or B are independently a bicyclic ring optionally substituted with one or more R¹.

In some embodiments, A and B are independently a tricyclic ring, e.g., tricyclic cycloalkyl, tricyclic heterocyclyl, tricyclic aryl, or tricyclic heteroaryl. The tricyclic ring may be saturated, partially unsaturated, or fully unsaturated (e.g., aromatic). In some embodiments, A or B are independently a tricyclic ring that comprises a fused, bridged, or spiro ring system, or a combination thereof. In some embodiments, A or B are independently a tricyclic ring comprising between 6 and 24 ring atoms (e.g., 6, 7, 8, 9, 10, 11, 12, 13, 14, 15, 16, 17, 18, 19, 20, 21, 22, 23, or 24 ring atoms). In some embodiments, A is an 8-membered tricyclic ring. In some embodiments, B is an 8-membered tricyclic ring. In some embodiments, A is a 9-membered tricyclic ring. In some embodiments, B is a 9-membered tricyclic ring. In some embodiments, A is a 10-membered tricyclic ring. In some embodiments, B is a 10-membered tricyclic ring. In some embodiments, A or B are independently a tricyclic ring optionally substituted with one or more R¹.

In some embodiments, A and B are independently monocyclic cycloalkyl, monocyclic heterocyclyl, monocyclic aryl, or monocyclic heteroaryl. In some embodiments, A or B are independently bicyclic cycloalkyl, bicyclic heterocyclyl, bicyclic aryl, or bicyclic heteroaryl. In some embodiments, A or B are independently tricyclic cycloalkyl, tricyclic heterocyclyl, tricyclic aryl, or tricyclic heteroaryl. In some embodiments, A is monocyclic heterocyclyl. In some embodiments, B is monocyclic heterocyclyl. In some embodiments, A is bicyclic heterocyclyl. In some embodiments, B is bicyclic heterocyclyl. In some embodiments, A is monocyclic heteroaryl. In some embodiments, B is monocyclic heteroaryl. In some embodiments, A is bicyclic heteroaryl. In some embodiments, B is bicyclic heteroaryl.

In some embodiments, A and B are independently a nitrogen-containing heterocyclyl, e.g., heterocyclyl comprising one or more nitrogen atom. The one or more nitrogen atom of the nitrogen-containing heterocyclyl may be at any position of the ring. In some embodiments, the nitrogen-containing heterocyclyl is monocyclic, bicyclic, or tricyclic. In some embodiments, A or B are independently heterocyclyl comprising at least 1, at least 2, at least 3, at least 4, at least 5, or at least 6 nitrogen atoms. In some embodiments, A is heterocyclyl comprising 1 nitrogen atom. In some embodiments, B is heterocyclyl comprising 1 nitrogen atom. In some embodiments, A is heterocyclyl comprising 2 nitrogen atoms. In some embodiments, B is heterocyclyl comprising 2 nitrogen atoms. In some embodiments, A is heterocyclyl comprising 3 nitrogen atoms. In some embodiments, B is heterocyclyl comprising 3 nitrogen atoms. In some embodiments, A is heterocyclyl comprising 4 nitrogen atoms. In some embodiments, B is heterocyclyl comprising 4 nitrogen atoms. In some embodiments, A or B are independently a nitrogen-containing heterocyclyl comprising one or more additional heteroatoms, e.g., one or more of oxygen, sulfur, boron, silicon, or phosphorus. In some embodiments, the one or more nitrogen of the nitrogen-containing heterocyclyl is substituted, e.g., with R¹.

In some embodiments, A and B are independently a nitrogen-containing heteroaryl, e.g., heteroaryl comprising one or more nitrogen atom. The one or more nitrogen atom of the nitrogen-containing heteroaryl may be at any position of the ring. In some embodiments, the nitrogen-containing heteroaryl is monocyclic, bicyclic, or tricyclic. In some embodiments, A or B are independently heteroaryl comprising at least 1, at least 2, at least 3, at least 4, at least 5, or at least 6 nitrogen atoms. In some embodiments, A is heteroaryl comprising 1 nitrogen atom. In some embodiments, B is heteroaryl comprising 1 nitrogen atom. In some embodiments, A is heteroaryl comprising 2 nitrogen atoms. In some embodiments, B is heteroaryl comprising 2 nitrogen atoms. In some embodiments, A is heteroaryl comprising 3 nitrogen atoms. In some embodiments, B is heteroaryl comprising 3 nitrogen atoms. In some embodiments, A is heteroaryl comprising 4 nitrogen atoms. In some embodiments, B is heteroaryl comprising 4 nitrogen atoms. In some embodiments, A or B are independently a nitrogen-containing heteroaryl comprising one or more additional heteroatoms, e.g., one or more of oxygen, sulfur, boron, silicon, or phosphorus. In some embodiments, the one or more nitrogen of the nitrogen-containing heteroaryl is substituted, e.g., with R¹.

In some embodiments, A is a 6-membered nitrogen-containing heterocyclyl, e.g., a 6-membered heterocyclyl comprising one or more nitrogen. In some embodiments, A is a 6-membered heterocyclyl comprising 1 nitrogen atom. In some embodiments, A is a 6-membered heterocyclyl comprising 2 nitrogen atoms. In some embodiments, A is a 6-membered heterocyclyl comprising 3 nitrogen atoms. In some embodiments, A is a 6-membered heterocyclyl comprising 4 nitrogen atoms. The one or more nitrogen atom of the 6-membered nitrogen-containing heterocyclyl may be at any position of the ring. In some embodiments, A is a 6-membered nitrogen-containing heterocyclyl optionally substituted with one or more R¹. In some embodiments, the one or more nitrogen of the 6-membered nitrogen-containing heterocyclyl is substituted, e.g., with R¹. In some embodiments, A is a 6-membered nitrogen-containing heterocyclyl comprising one or more additional heteroatoms, e.g., one or more of oxygen, sulfur, boron, silicon, or phosphorus.

In some embodiments, B is a 5-membered nitrogen-containing heterocyclyl or heteroaryl, e.g., a 5-membered heterocyclyl or heteroaryl comprising one or more nitrogen. In some embodiments, B is a 5-membered heterocyclyl comprising 1 nitrogen atom. In some embodiments, B is a 5-membered heteroaryl comprising 1 nitrogen atom. In some embodiments, B is a 5-membered heterocyclyl comprising 2 nitrogen atoms. In some embodiments, B is a 5-membered heteroaryl comprising 2 nitrogen atoms. In some embodiments, B is a 5-membered heterocyclyl comprising 3 nitrogen atoms. In some embodiments, B is a 5-membered heteroaryl comprising 3 nitrogen atoms. The one or more nitrogen atom of the 5-membered nitrogen-containing heterocyclyl or heteroaryl may be at any position of the ring. In some embodiments, B is a 5-membered nitrogen-containing heterocyclyl optionally substituted with one or more R¹. In some embodiments, B is a 5-membered nitrogen-containing heteroaryl optionally substituted with one or more R². In some embodiments, the one or more nitrogen of the 5-membered nitrogen-containing heterocyclyl or heteroaryl is substituted, e.g., with R¹. In some embodiments, B is a 5-membered nitrogen-containing heterocyclyl or heteroaryl comprising one or more additional heteroatoms, e.g., one or more of oxygen, sulfur, boron, silicon, or phosphorus.

For each of Formulas (I), (II), (III), or (IV), in some embodiments, each of A and B are independently selected from:

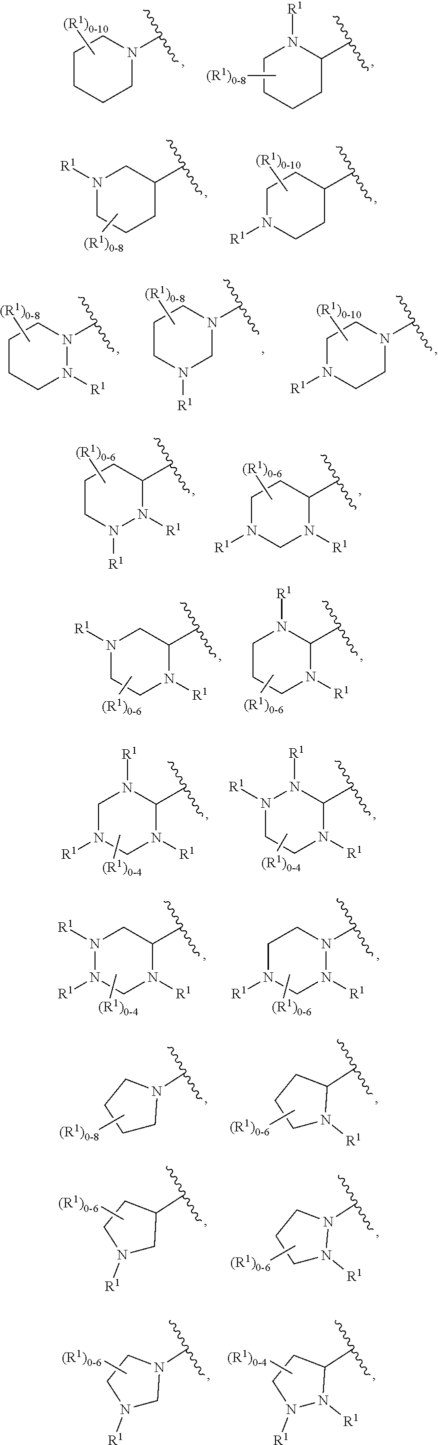

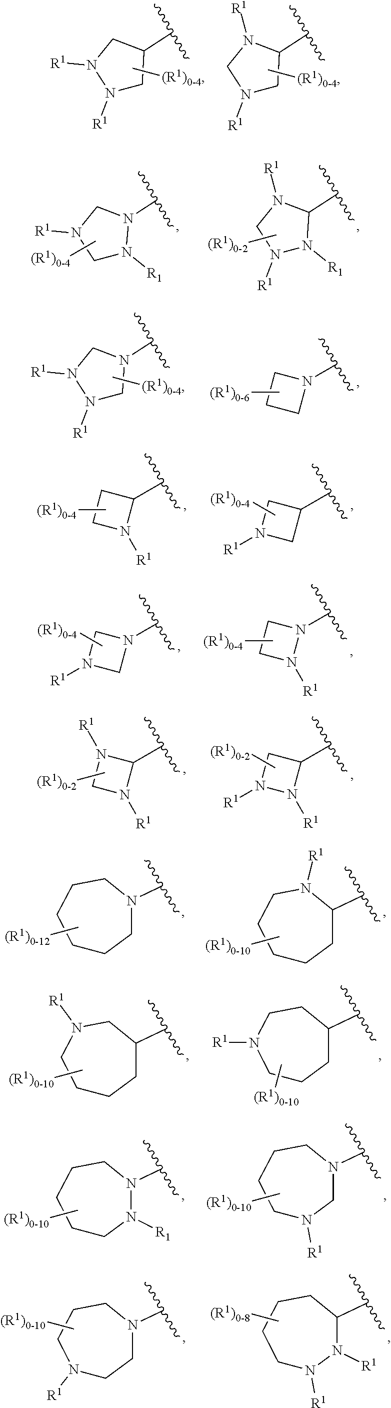

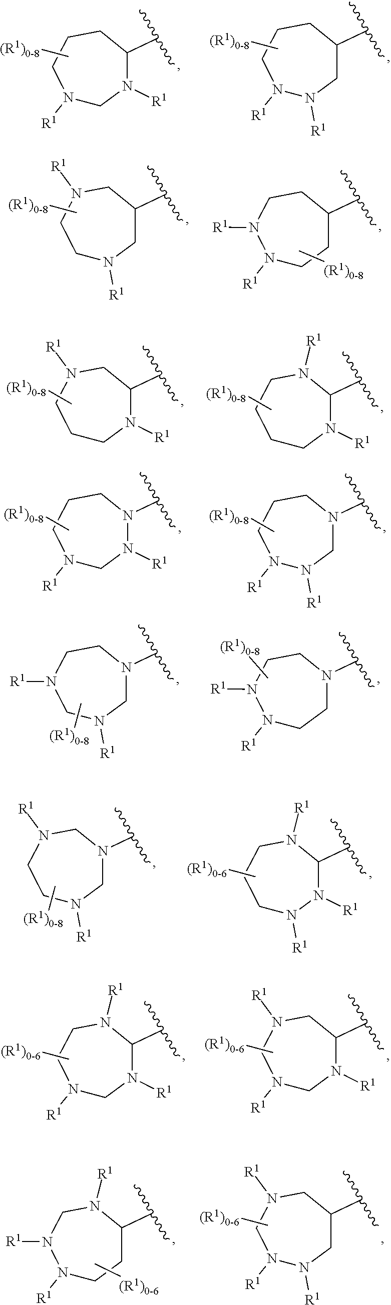

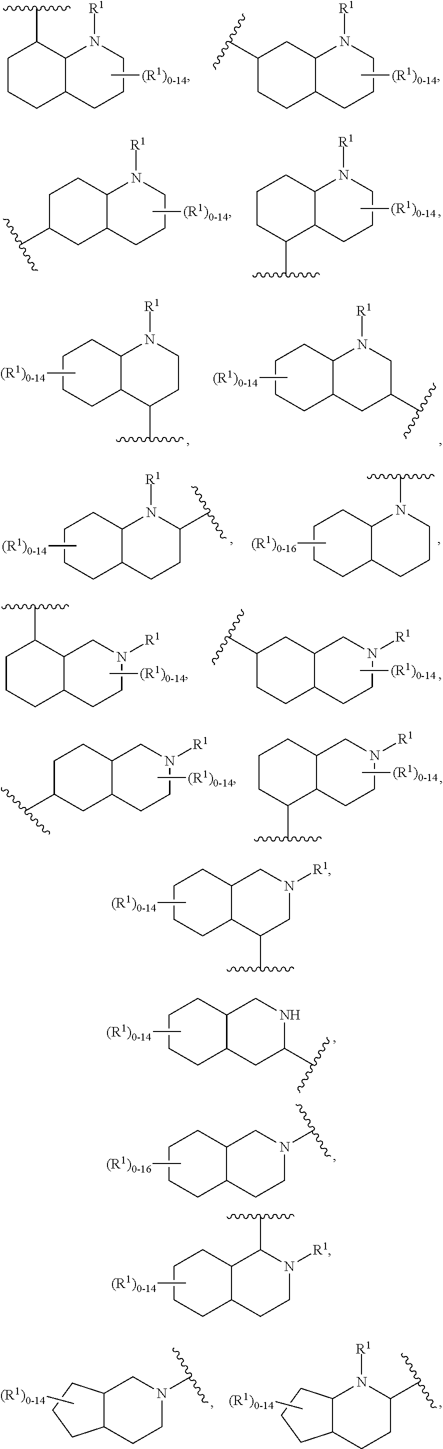

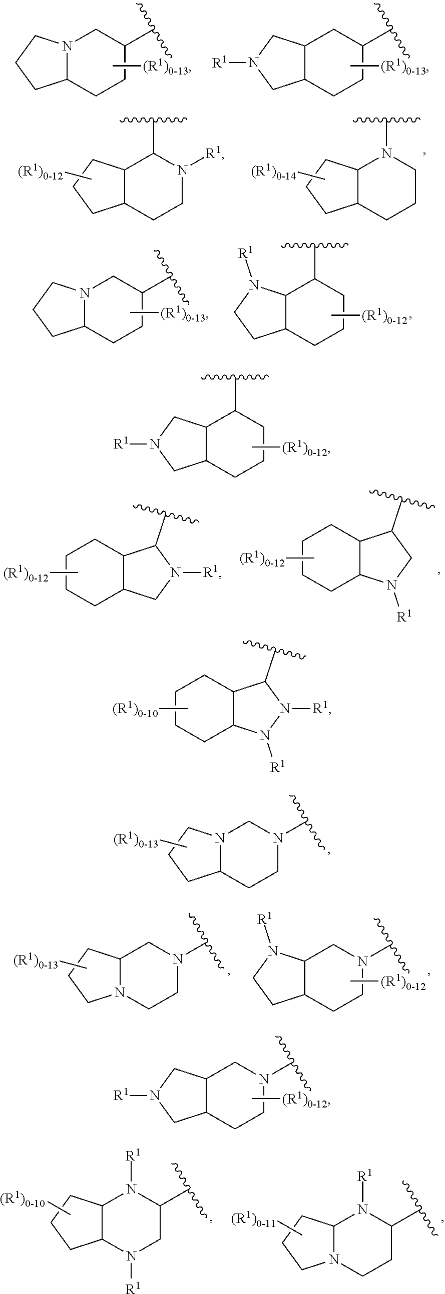

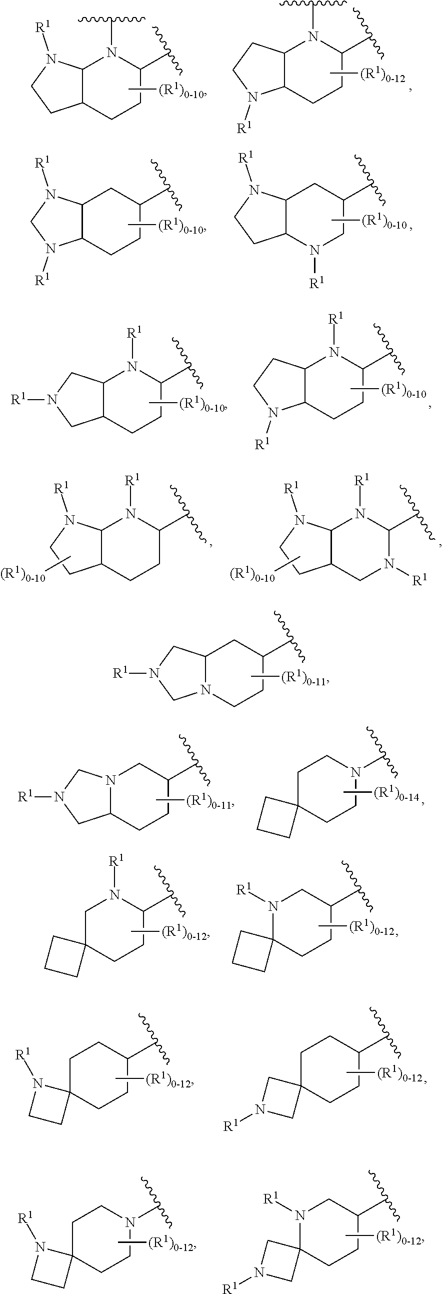

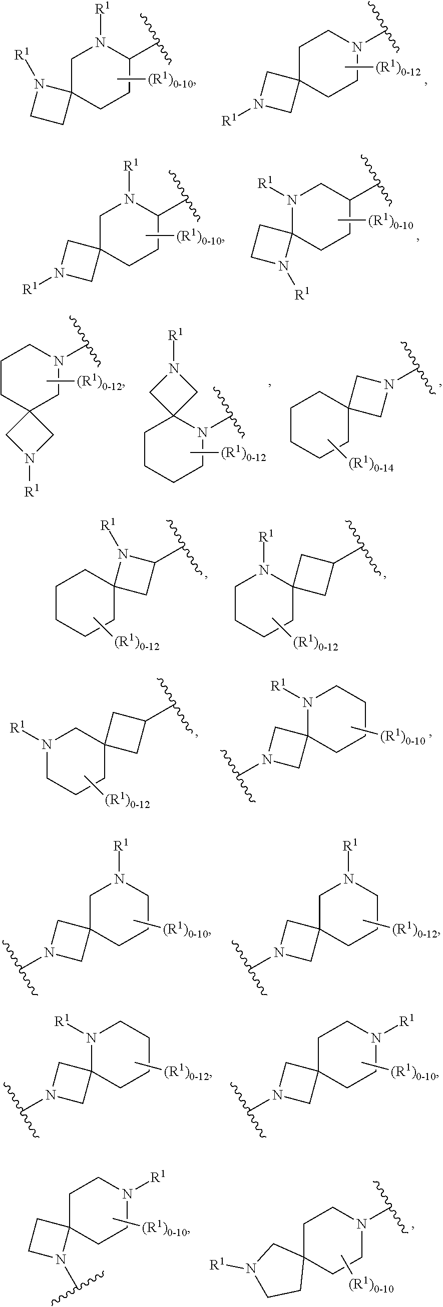

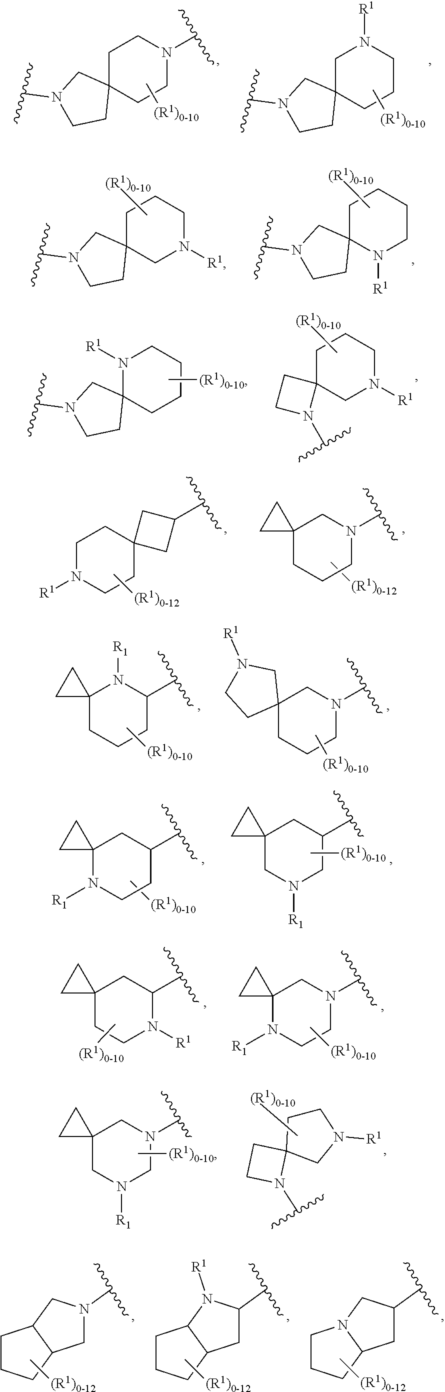

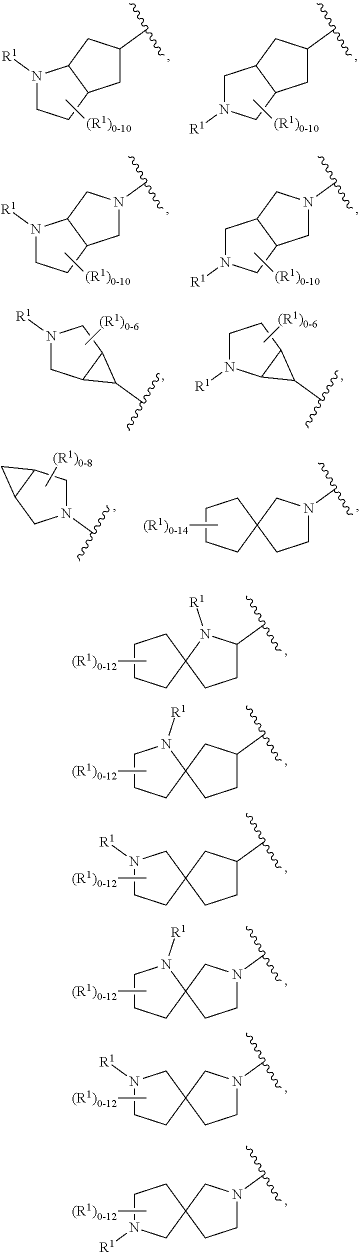

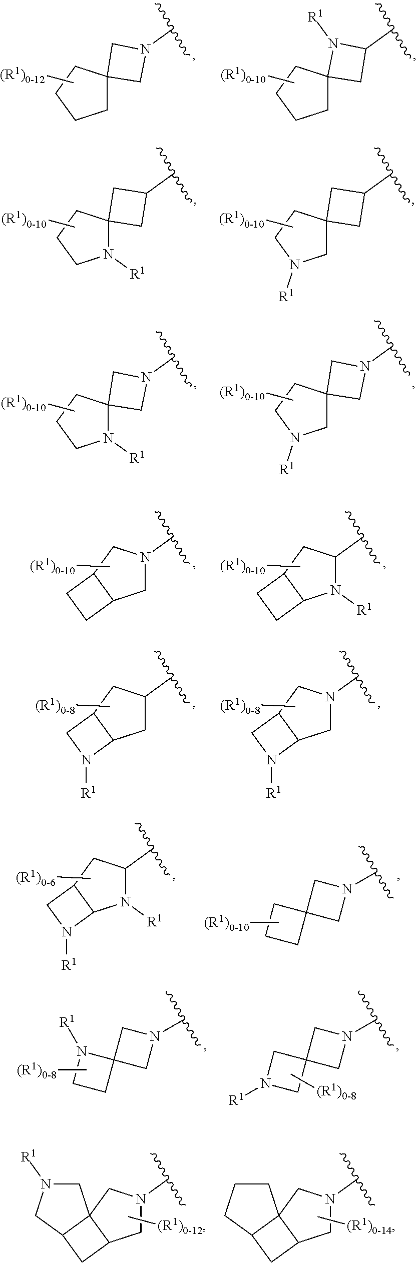

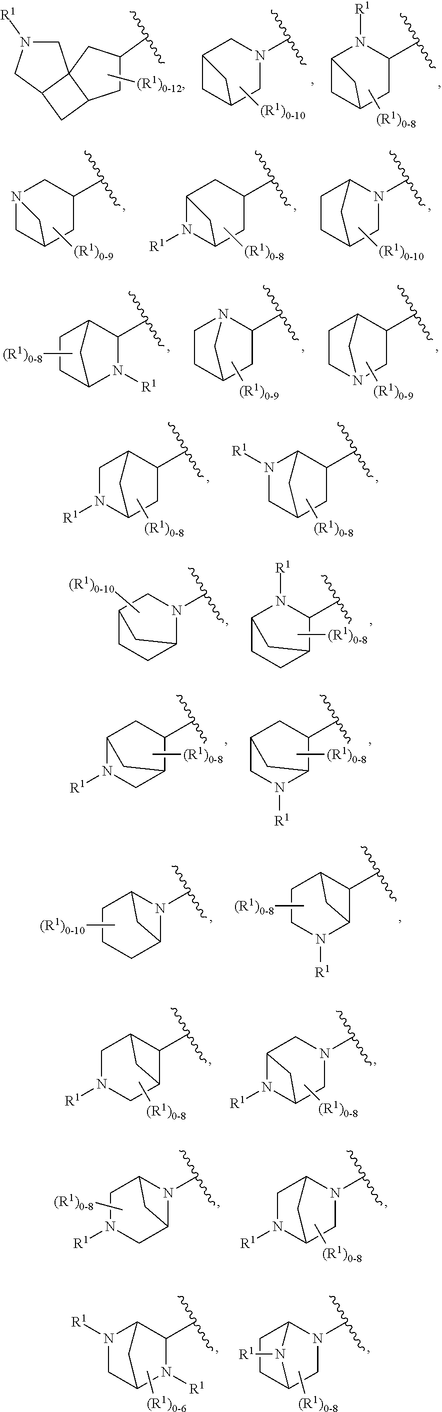

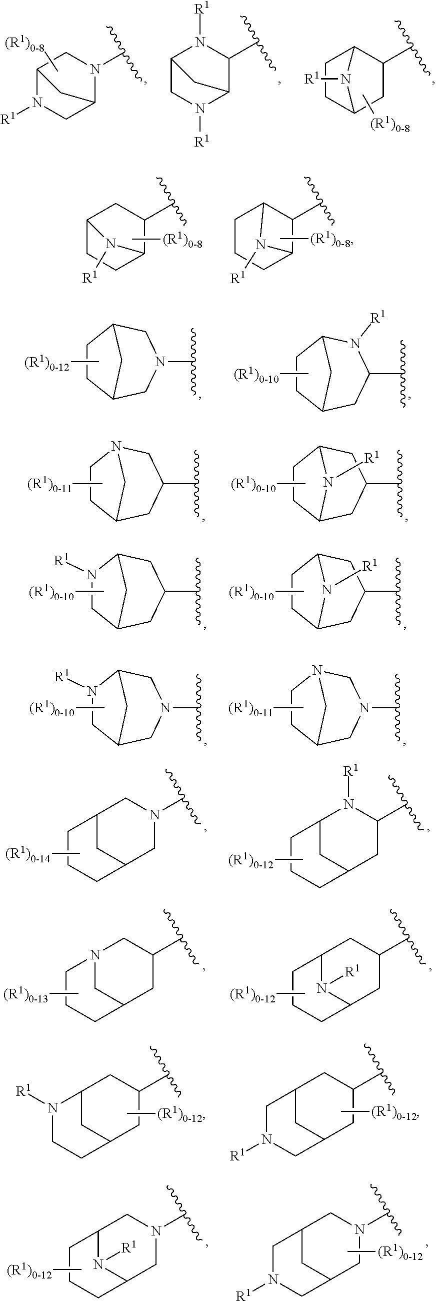

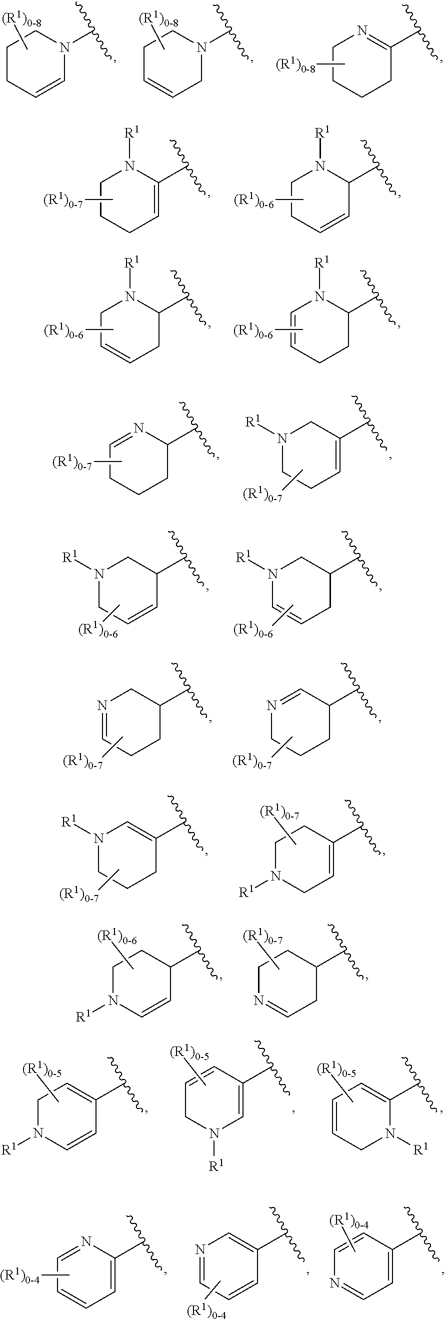

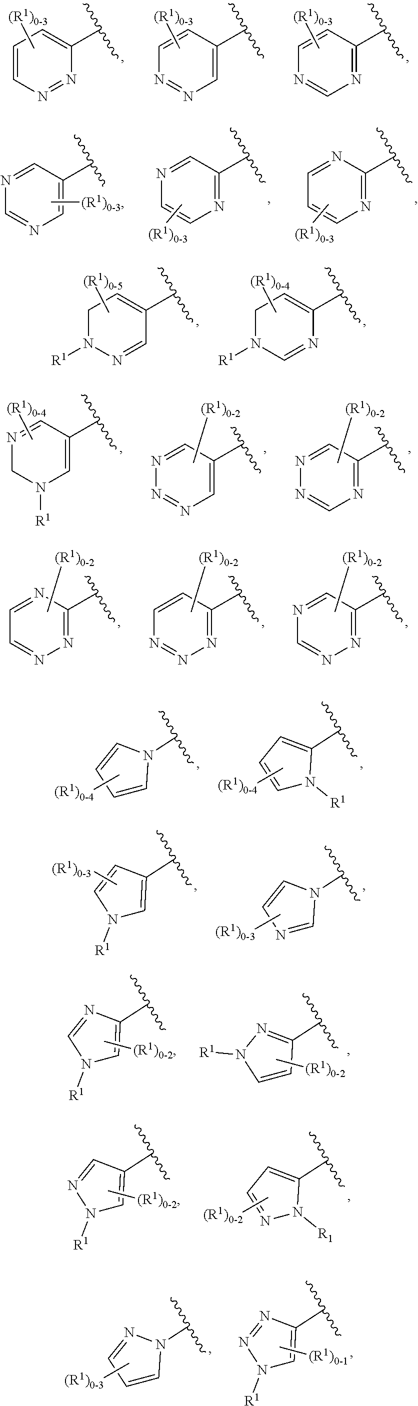

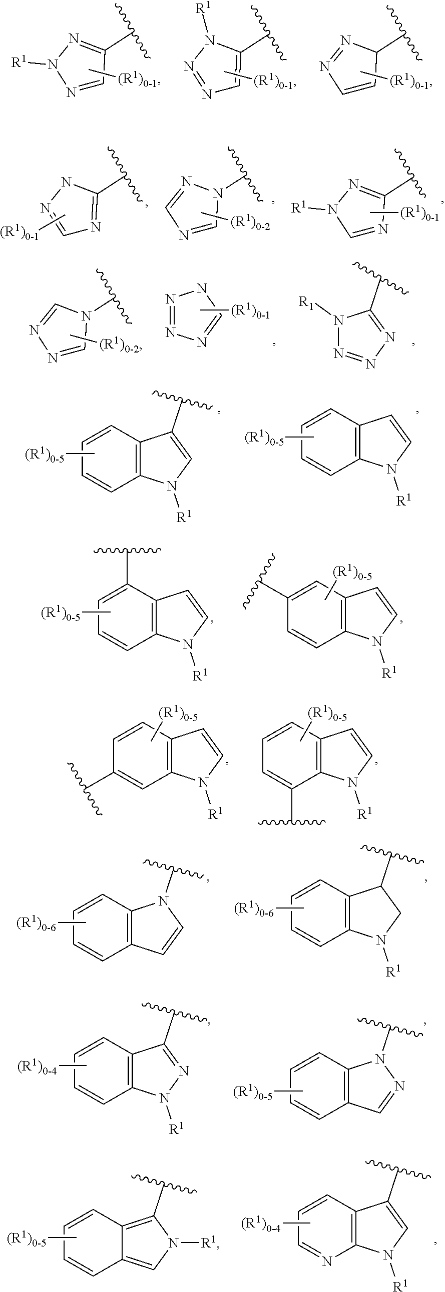

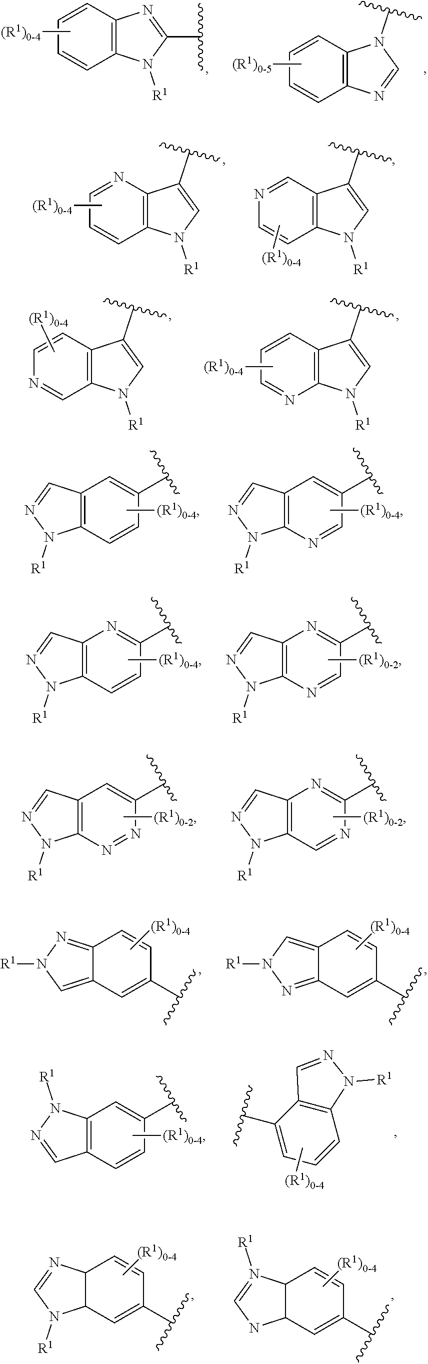

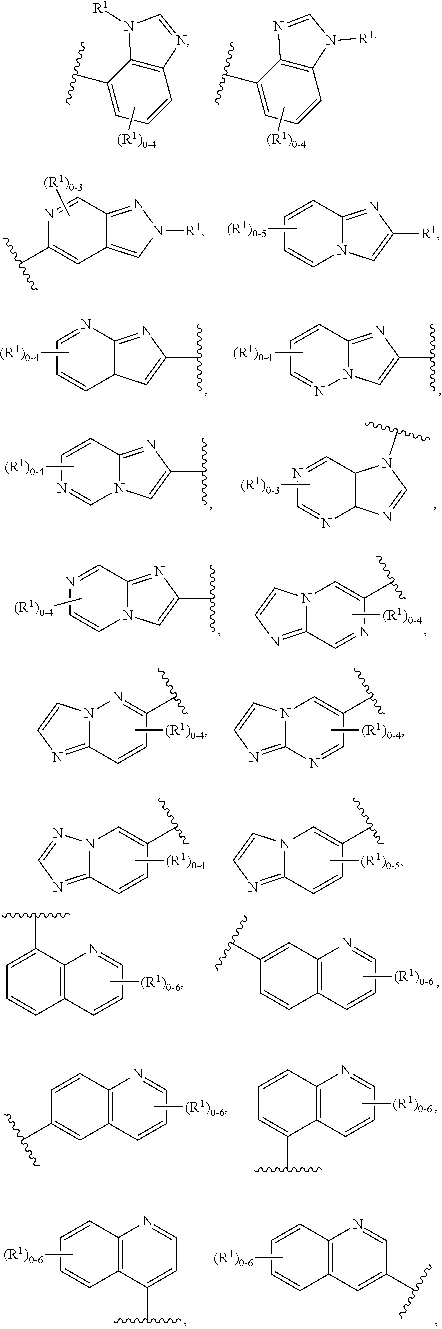

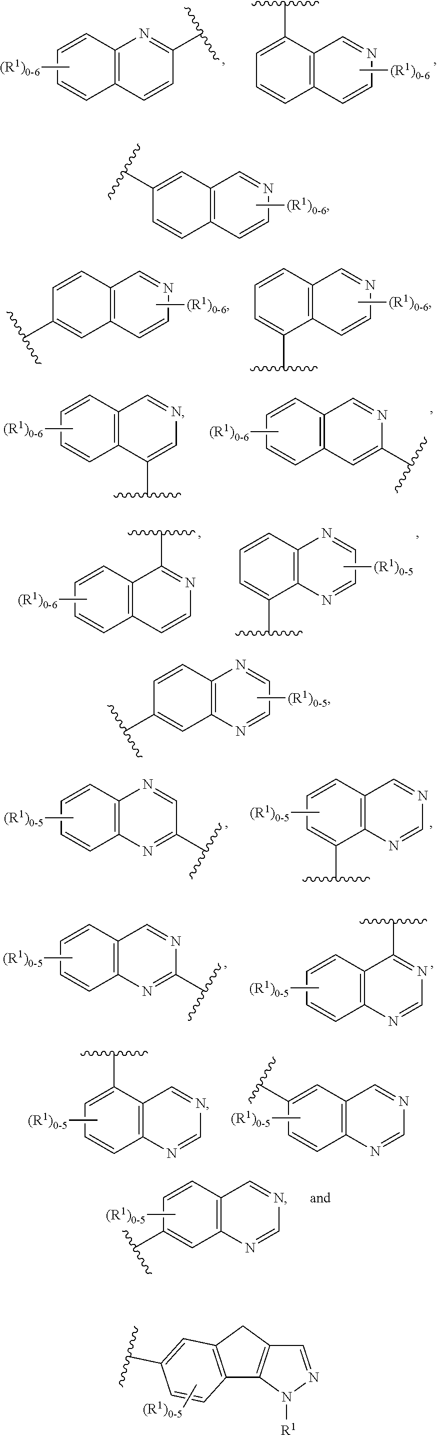

wherein each R¹ is as defined herein. In an embodiment, A and B are each independently a saturated, partially saturated, or unsaturated (e.g., aromatic) derivative of one of the rings described above. In an embodiment, A and B are each independently a stereoisomer of one of the rings described above.

In some embodiments, each of A and B are independently selected from:

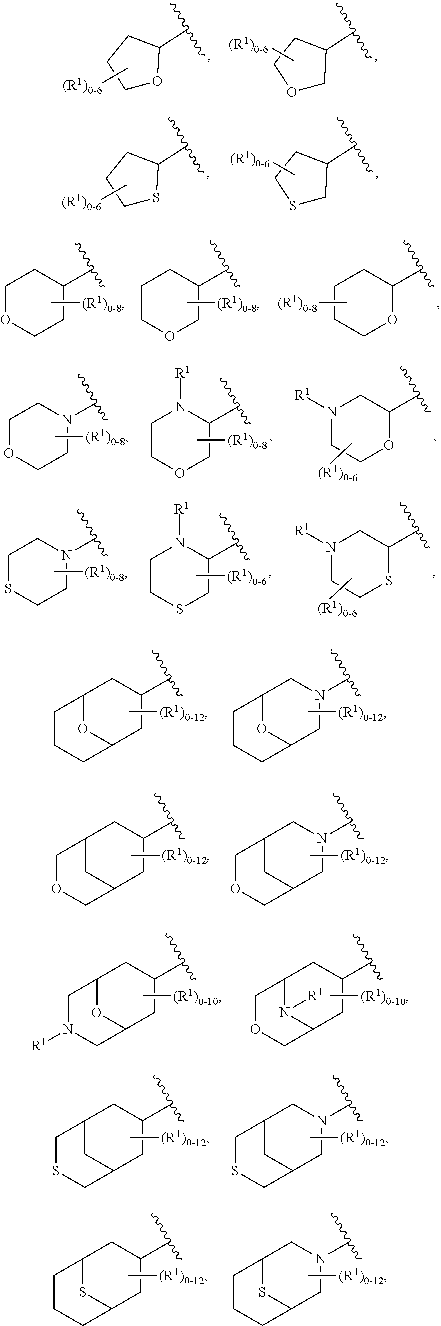

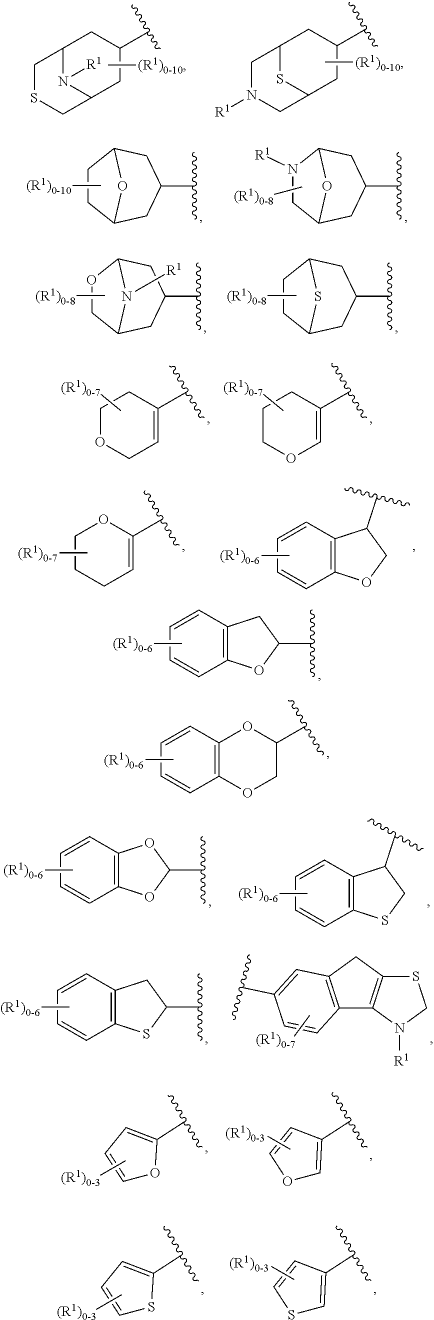

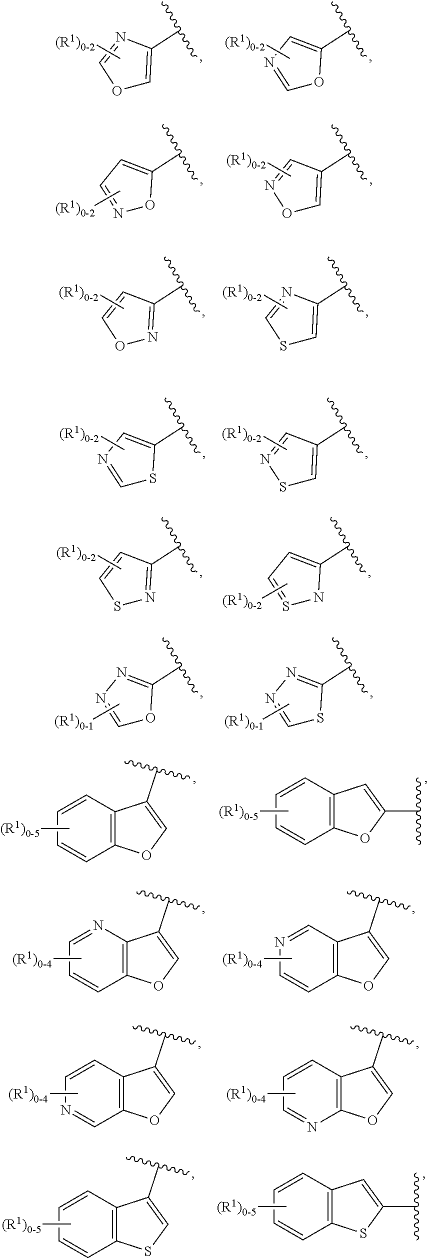

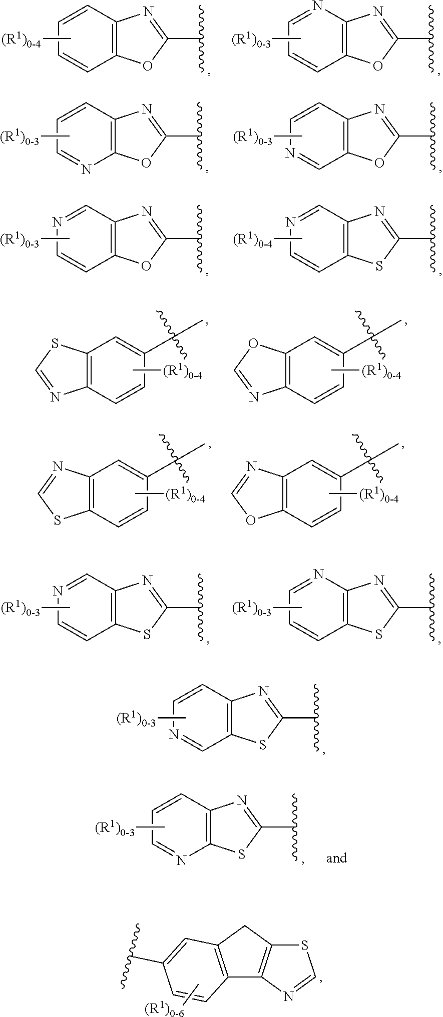

wherein each R¹ is as defined herein. In an embodiment, A and B are each independently a saturated, partially saturated, or unsaturated (e.g., aromatic) derivative of one of the rings described above. In an embodiment, A and B are each independently a stereoisomer of one of the rings described above.

For each of Formulas (I), (II), (III), or (IV), in some embodiments, one of A and B is independently a monocyclic heteroaryl or bicyclic heteroaryl, each of which is optionally substituted with one or more R¹. In some embodiments, one of A and B is independently a bicyclic heteroaryl optionally substituted with one or more R¹. In some embodiments, one of A and B is independently a nitrogen-containing heteroaryl optionally substituted with one or more R¹. In some embodiments, one of A and B is independently selected from

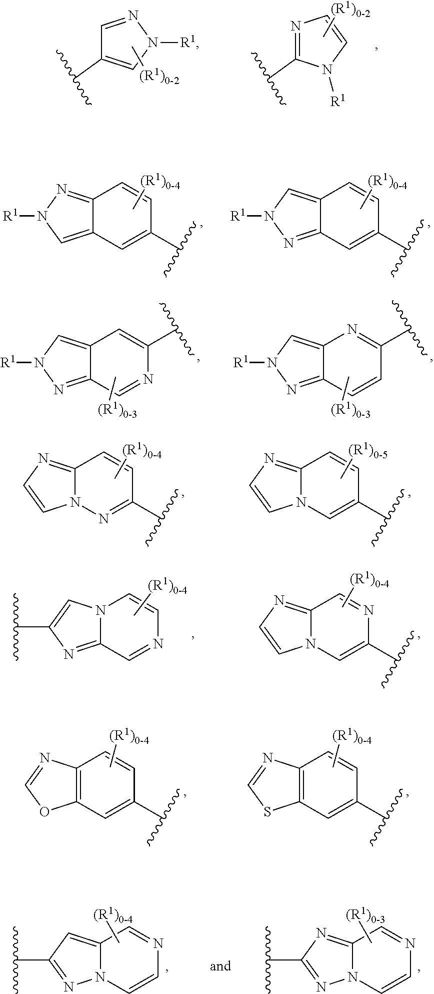

wherein R¹ is as described herein. In some embodiments, one of A and B is independently selected from

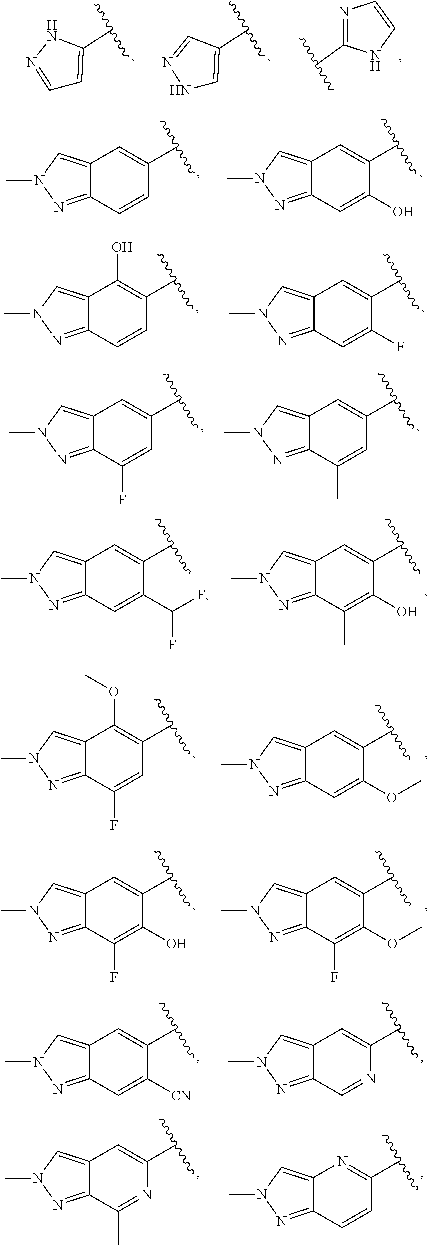

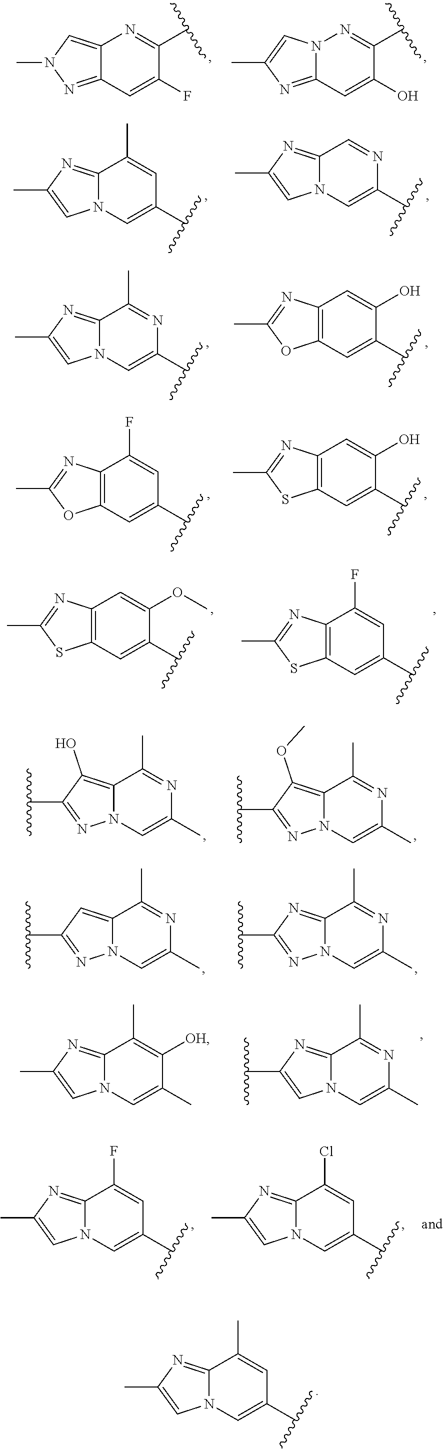

In some embodiments, one of A and B is independently a monocyclic heterocyclyl or bicyclic heterocyclyl, each of which is optionally substituted with one or more R¹. In some embodiments, one of A and B is independently a nitrogen-containing heterocyclyl optionally substituted with one or more R¹. In some embodiments, one of A and B is independently a 4-8 membered heterocyclyl optionally substituted with one or more R¹. In some embodiments, one of A and B is independently selected from

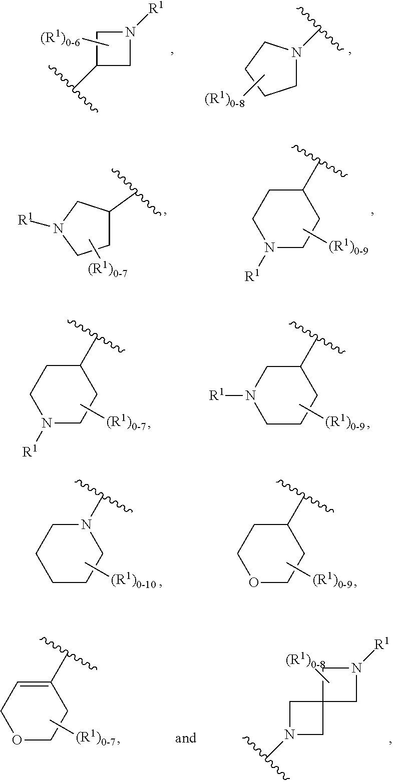

wherein R¹ is as described herein. In some embodiments, one of A and B is independently selected from one of A and B is independently is selected from

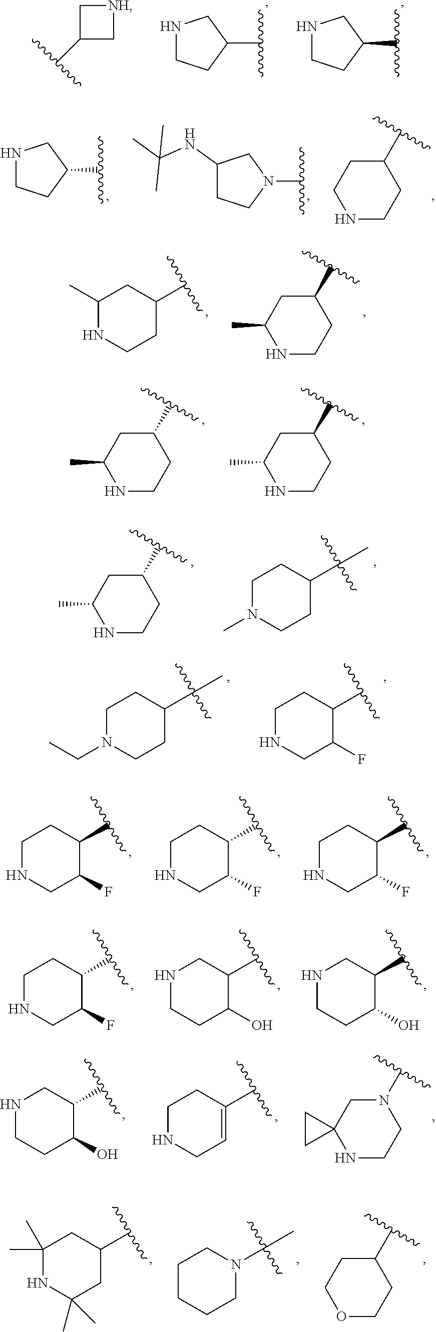

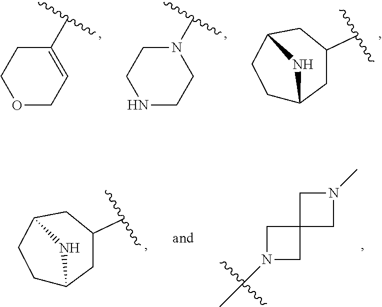

For each of Formulas (I), (II), (III), or (IV), in some embodiments, A is a monocyclic heteroaryl or bicyclic heteroaryl, each of which is optionally substituted with one or more R¹. In some embodiments, A is a bicyclic heteroaryl optionally substituted with one or more R¹. In some embodiments, A is a nitrogen-containing heteroaryl optionally substituted with one or more R¹. In some embodiments, A is selected from

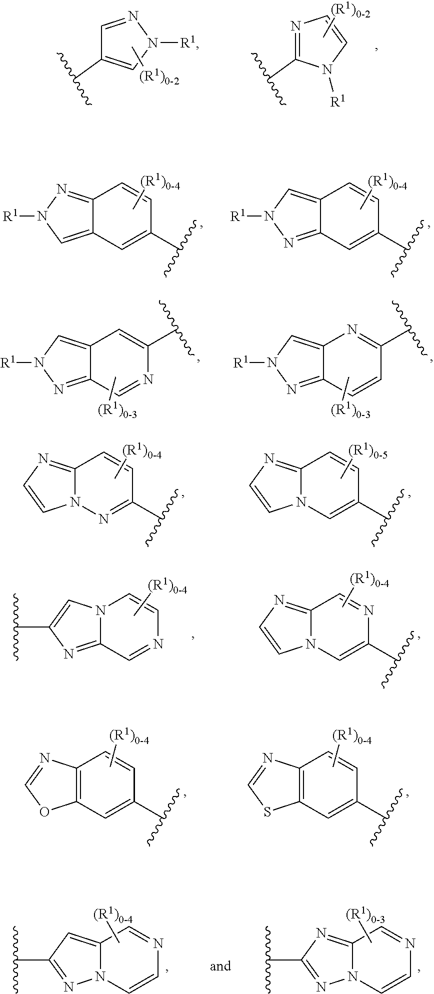

wherein R¹ is as described herein. In some embodiments, A is selected from

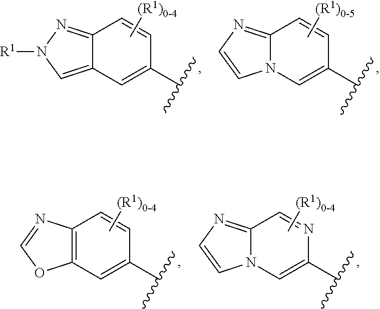

wherein R¹ is as described herein. In some embodiments, A is

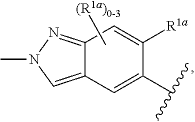

wherein each R^{1a} is independently C₁-C₆-alkyl, C₁-C₆-heteroalkyl, C₁-C₆-haloalkyl, halo, cyano, or —OR^{A}, and each alkyl, heteroalkyl, and haloalkyl is optionally substituted with one or more R⁷. In some embodiments, at least one of R^{1a} is C₁-C₆-alkyl, halo, or —OR^{A}. In some embodiments, R^{1a} is —OR^{A} and R^{A} is H. In some embodiments, R^{1a} is halo.

In some embodiments, A is selected from

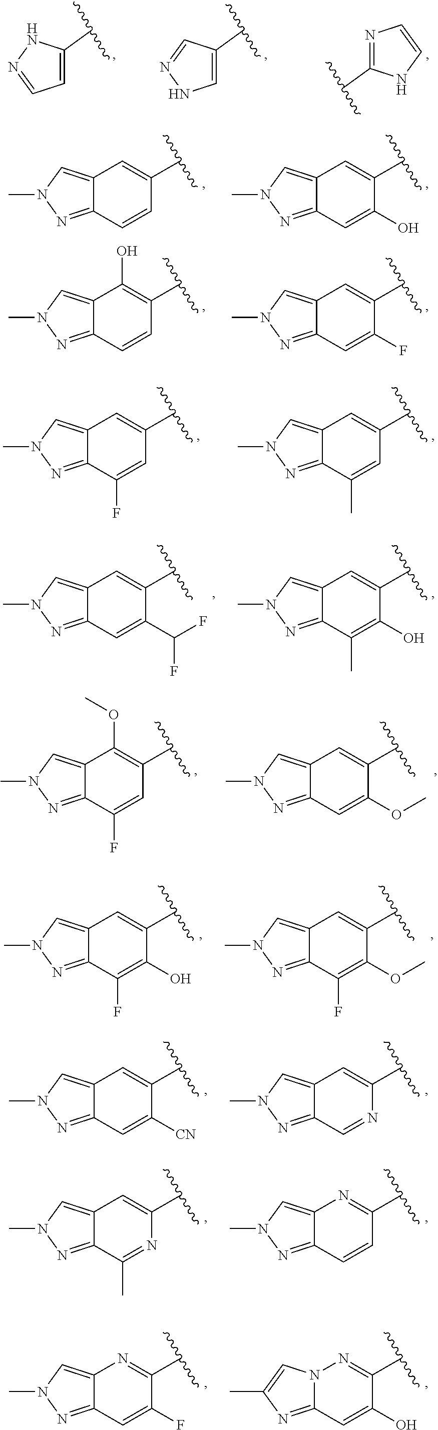

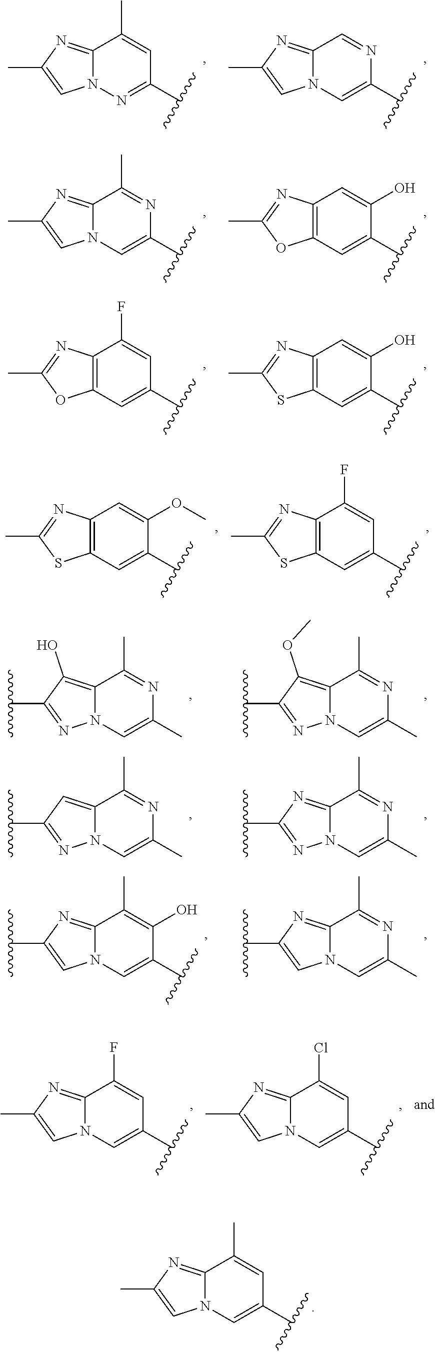

In some embodiments, A is selected from

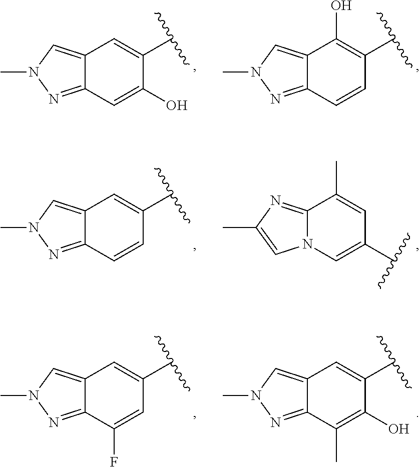 [SMILES: *c1cc2cn(C)nc2cc1O ; *c1ccc2nn(C)cc2c1O ; *c1ccc2nn(C)cc2c1 ; *c1cc(C)c2nc(C)cn2c1 ; *c1cc(F)c2nn(C)cc2c1 ; *c1cc2cn(C)nc2c(C)c1O]

In some embodiments, A is

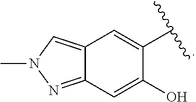 [SMILES: *c1cc2cn(C)nc2cc1O]

In some embodiments, A is

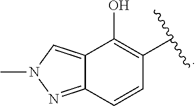 [SMILES: *c1ccc2nn(C)cc2c1O]

In some embodiments, A is

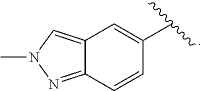 [SMILES: *c1ccc2nn(C)cc2c1]

In some embodiments, A is

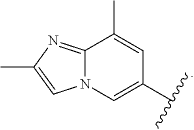 [SMILES: *c1cc(C)c2nc(C)cn2c1]

In some embodiments, A is

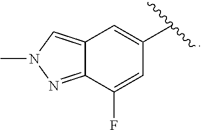 [SMILES: *c1cc(F)c2nn(C)cc2c1]

In some embodiments, A is

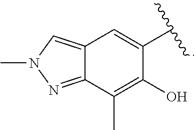 [SMILES: *c1cc2cn(C)nc2c(C)c1O]

In some embodiments, A is a monocyclic heterocyclyl or bicyclic heterocyclyl, each of which is optionally substituted with one or more R¹. In some embodiments, A is a nitrogen-containing heterocyclyl optionally substituted with one or more R¹. In some embodiments, A is a 4-8 membered heterocyclyl optionally substituted with one or more R¹.

In some embodiments, A is selected from

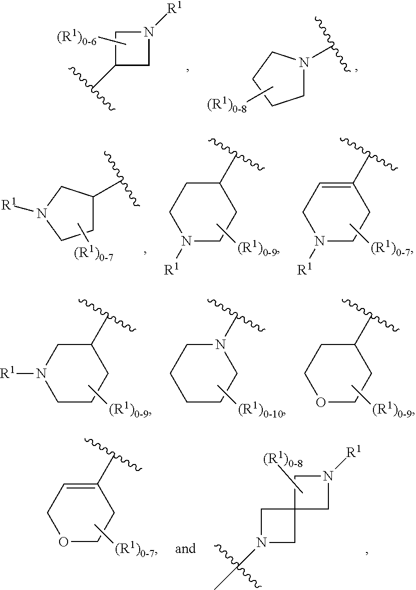

wherein R¹ is as described herein. In some embodiments, A is selected from

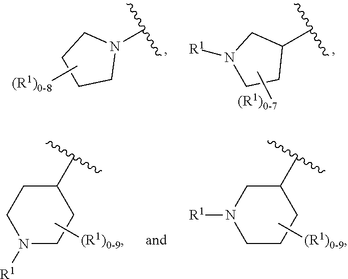

and, wherein R¹ is as described herein. In some embodiments, A is

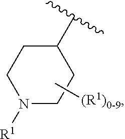

wherein R¹ is as described herein.

In some embodiments, A is selected from

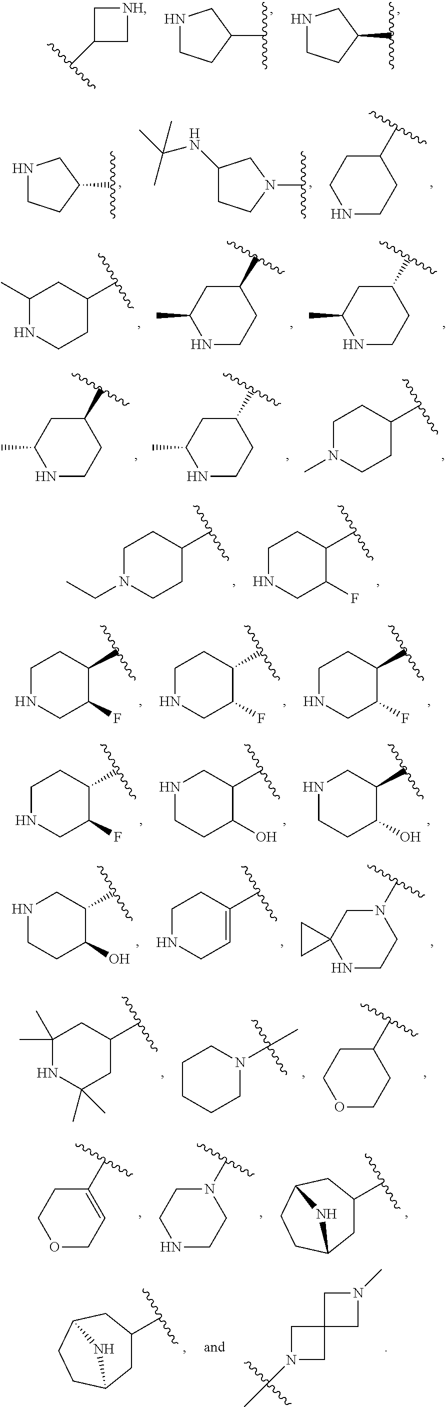

In some embodiments, A is selected from

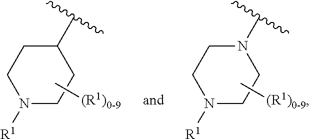

wherein R¹ is as defined herein In some embodiments, A is A is

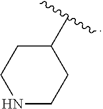 [SMILES: *C1CCNCC1]

In some embodiments, A is

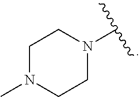 [SMILES: *N1CCN(C)CC1]

In some embodiments, A is

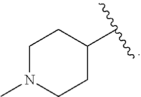 [SMILES: *C1CCN(C)CC1]

In some embodiments, A is selected from

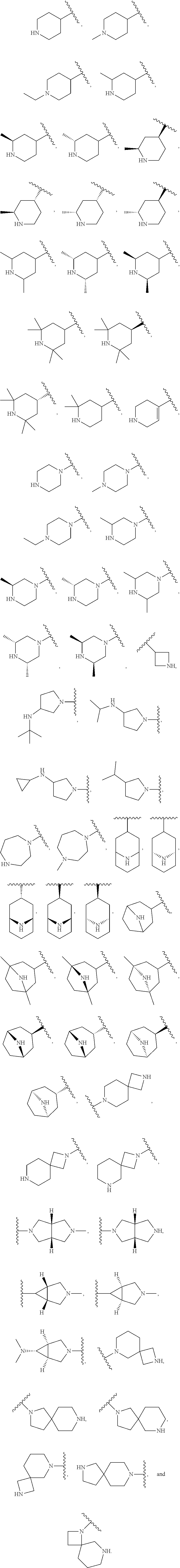

For each of Formulas (I), (II), (III), or (IV), in some embodiments, B is a monocyclic heteroaryl or bicyclic heteroaryl, each of which is optionally substituted with one or more R¹. In some embodiments, B is a bicyclic heteroaryl optionally substituted with one or more R¹. In some embodiments, B is a nitrogen-containing heteroaryl optionally substituted with one or more R¹. In some embodiments, B is selected from

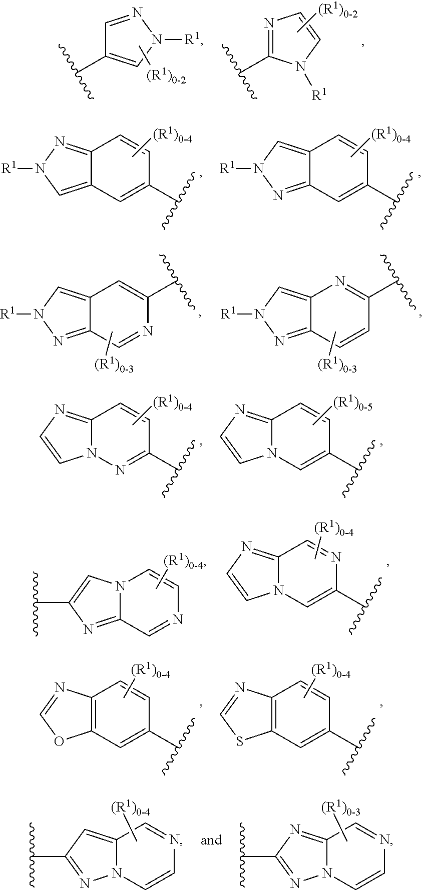

wherein R¹ is as described herein. In some embodiments, B is selected from

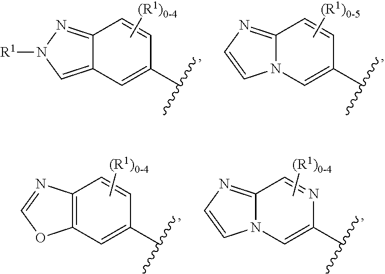

wherein R¹ is as described herein. In some embodiments, B is

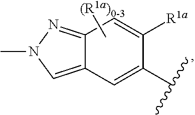

wherein each R^{1a} is independently C₁-C₆-alkyl, C₁-C₆-heteroalkyl, C₁-C₆-haloalkyl, halo, cyano, or —OR^{A}, and each alkyl, heteroalkyl, and haloalkyl is optionally substituted with one or more R⁷. In some embodiments, at least one of R^{1a} is C₁-C₆-alkyl, halo, or —OR^{A}. In some embodiments, R^{1a} is —OR^{A} and R^{A} is H. In some embodiments, R^{1a} is halo.

In some embodiments, B is selected from

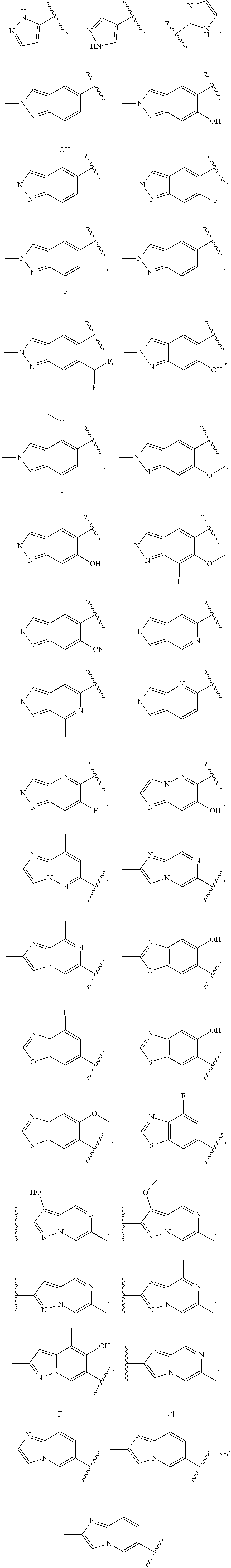

In some embodiments, B is selected from

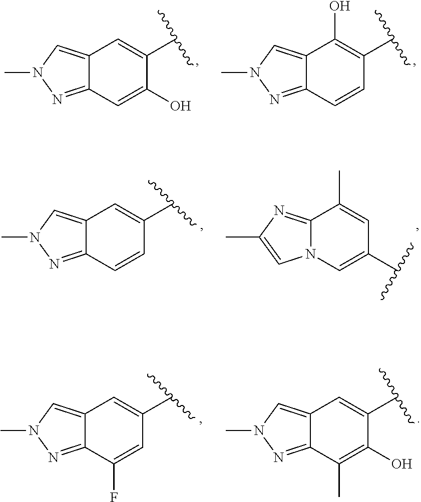 [SMILES: *c1cc2cn(C)nc2cc1O ; *c1ccc2nn(C)cc2c1O ; *c1ccc2nn(C)cc2c1 ; *c1cc(C)c2nc(C)cn2c1 ; *c1cc(F)c2nn(C)cc2c1 ; *c1cc2cn(C)nc2c(C)c1O]

In some embodiments, B is

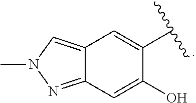 [SMILES: *c1cc2cn(C)nc2cc1O]

In some embodiments, B is

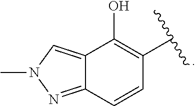 [SMILES: *c1ccc2nn(C)cc2c1O]

In some embodiments, B is

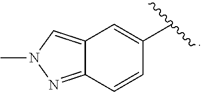 [SMILES: *c1ccc2nn(C)cc2c1]

In some embodiments, B is

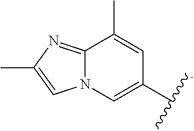 [SMILES: *c1cc(C)c2nc(C)cn2c1]

In some embodiments, B is

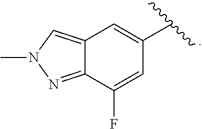 [SMILES: *c1cc(F)c2nn(C)cc2c1]

In some embodiments, B is

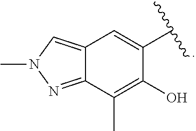 [SMILES: *c1cc2cn(C)nc2c(C)c1O]

In some embodiments, B is

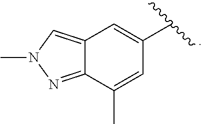 [SMILES: *c1cc(C)c2nn(C)cc2c1]

In some embodiments, B is

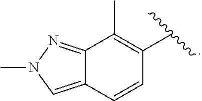 [SMILES: *c1ccc2cn(C)nc2c1C]

In some embodiments, B is

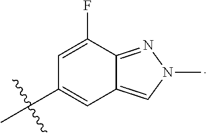 [SMILES: *c1cc(F)c2nn(C)cc2c1]

In some embodiments, B is

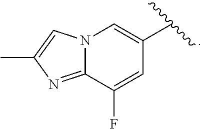 [SMILES: *c1cc(F)c2nc(C)cn2c1]

In some embodiments, B is

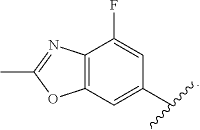 [SMILES: *c1cc(F)c2nc(C)oc2c1]

In some embodiments, B is

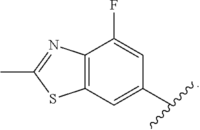 [SMILES: *c1cc(F)c2nc(C)sc2c1]

In some embodiments, B is

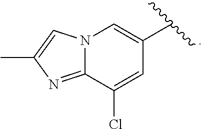 [SMILES: *c1cc(Cl)c2nc(C)cn2c1]

In some embodiments, B is

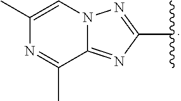 [SMILES: *c1nc2c(C)nc(C)cn2n1]

In some embodiments, B is

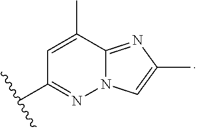 [SMILES: *c1cc(C)c2nc(C)cn2n1]

In some embodiments, B is

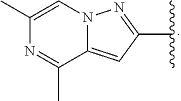 [SMILES: *c1cc2c(C)nc(C)cn2n1]

In some embodiments, B is a monocyclic heterocyclyl or bicyclic heterocyclyl, each of which is optionally substituted with one or more R¹. In some embodiments, B is a nitrogen-containing heterocyclyl optionally substituted with one or more R¹. In some embodiments, B is a 4-8 membered heterocyclyl optionally substituted with one or more R¹. In some embodiments, B is selected from

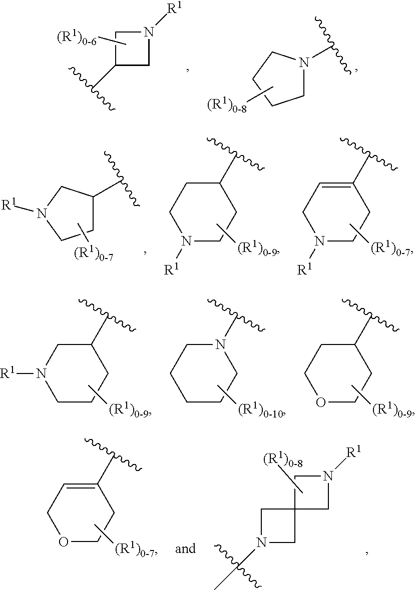

wherein R¹ is as described herein. In some embodiments, B is selected from

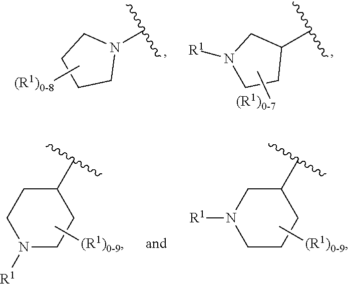

and, wherein R¹ is as described herein. In some embodiments, B is

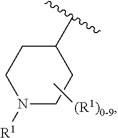

wherein R¹ is as described herein.

In some embodiments, B is selected from

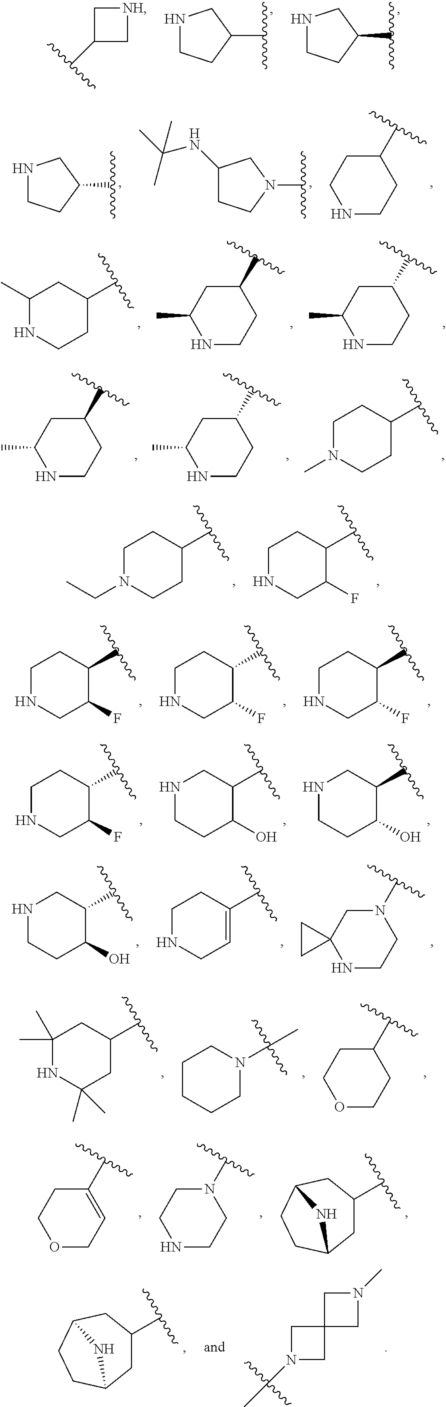

In some embodiments, B is selected from

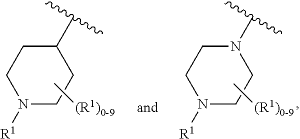

wherein R¹ is as defined herein In some embodiments, B is

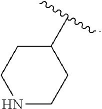 [SMILES: *C1CCNCC1]

In some embodiments, B is

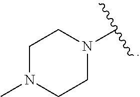 [SMILES: *N1CCN(C)CC1]

In some embodiments, B is

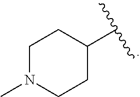 [SMILES: *C1CCN(C)CC1]

In some embodiments, B is selected from

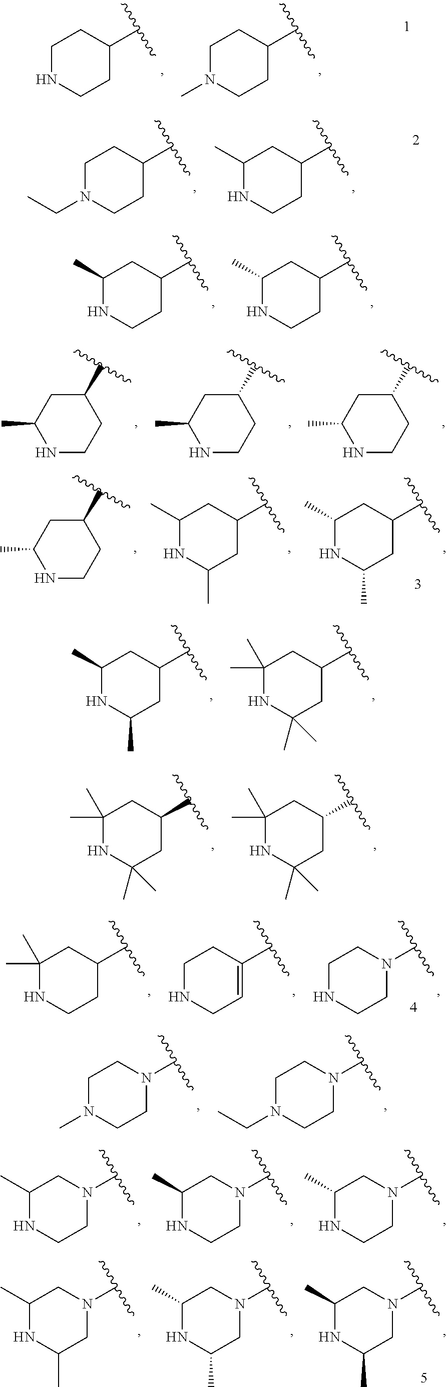

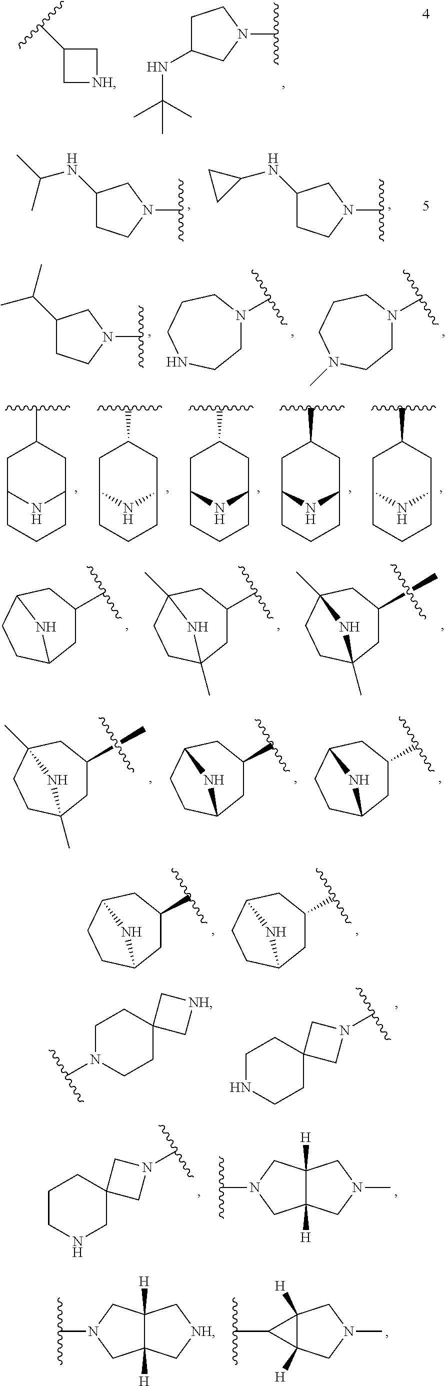

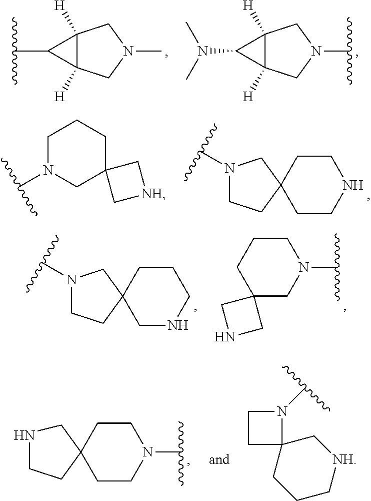

In some embodiments B is selected from

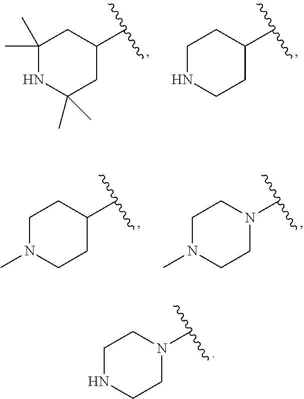

In some embodiments, B is

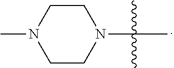 [SMILES: *N1CCN(C)CC1]

In some embodiments, B is

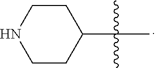 [SMILES: *C1CCNCC1]

In some embodiments, A is substituted with 0 or 1 R¹. In some embodiments, B is substituted with 0, 1, or 2 R¹. In some embodiments, R¹ is C₁-C₆-alkyl, —OR^{A}, or halo (e.g., CH₃, OH, or F). In some embodiments, R¹ is CH₃. In some embodiments, R¹ is OH. In some embodiments, R¹ is F.

As generally described for Formulas (I), (II), (III), or (IV), L¹ may be absent, C₁-C₆-alkylene, C₁-C₆-heteroalkylene, —O—, —C(O)—, —N(R³)—, —N(R³)C(O)—, or —C(O)N(R³)—, wherein each alkylene and heteroalkylene is optionally substituted with one or more R⁴. For each of Formulas (I), (II), (III), or (IV), in some embodiments, L¹ is absent or —N(R³)— (e.g., —N(CH₃)—). In some embodiments, L¹ is absent. In some embodiments, L¹ is —N(R³)— (e.g., —N(CH₃)—).

As generally described for Formulas (I), (II), (III), or (IV), L² may be absent, C₁-C₆-alkylene, C₁-C₆-heteroalkylene, C₆-C₁₂-arylene, C₅-C₁₂-heteroarylene, —O—, —C(O)—, —N(R³)—, —N(R³)C(O)—, or —C(O)N(R³)—, wherein each alkylene, heteroalkylene, arylene, and heteroarylene is optionally substituted with one or more R⁴. In some embodiments, L² is absent, C₆-C₁₂-arylene, or C₅-C₁₂-heteroarylene. In some embodiments, L² is absent. In some embodiments, L² is C₆-C₁₂-arylene. In some embodiments, L² is C₆-C₁₂-heteroarylene.

As generally described for Formula (I), W and Y each may be independently C(R⁵) or N and X may be C or N, wherein at least one of W, X, and Y is N. In some embodiments, W is C(R⁵) (e.g., CH). In some embodiments, W is N. In some embodiments, Y is C(R⁵) (e.g., CH). In some embodiments, Y is N. In some embodiments, X is C. In some embodiments, X is N. In some embodiments, each of W and Y is independently C(R⁵) (e.g., CH). In some embodiments, each of W and Y is independently N. In some embodiments, each of Y and X is independently N. In some embodiments, each of X and W is independently N. In some embodiments, one of X and Y is independently N and W is N. In some embodiments, X and W is independent N and Y is N. In some embodiments, each of X, Y, and W is independently N.

As generally described for Formula (II), Y may be C(R⁵) or N. In some embodiments, Y is Y is C(R⁵) (e.g., CH). In some embodiments, Y is N.

As generally described for Formulas (I), (II), (III), or (IV), R² may be hydrogen, halo, cyano, C₁-C₆-alkyl, C₂-C₆-alkenyl, C₂-C₆-alkynyl, C₁-C₆-heteroalkyl, or C₁-C₆-haloalkyl. In some embodiments, R² is hydrogen. In some embodiments, R² is halogen (e.g., chloro).

In some embodiments, the compound of Formula (I) is a compound of Formula (I-a):

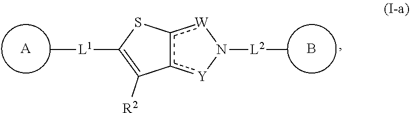

or a pharmaceutically acceptable salt, solvate, hydrate, tautomer, or stereoisomer thereof, wherein A and B are each independently cycloalkyl, heterocyclyl, aryl, or heteroaryl, each of which is optionally substituted with one or more R¹; L¹ is absent, C₁-C₆-alkylene, C₁-C₆-heteroalkylene, —O—, —C(O)—, —N(R³)—, —N(R³)C(O)—, or —C(O)N(R³)—, wherein each alkylene and heteroalkylene is optionally substituted with one or more R⁴; W and Y are each independently C(R⁵) or N; wherein the dashed lines in the ring comprising W, N, and Y may be single or double bonds as valency permits; each R¹ is independently hydrogen, C₁-C₆-alkyl, C₂-C₆-alkenyl, C₂-C₆-alkynyl, C₁-C₆-heteroalkyl, C₁-C₆-haloalkyl, cycloalkyl, heterocyclyl, aryl, C₁-C₆ alkylene-aryl, C₁-C₆ alkenylene-aryl, C₁-C₆ alkylene-heteroaryl, heteroaryl, halo, cyano, oxo, —OR^{A}, —NR^{B}R^{C}, —NR^{B}C(O)R^{D}, —NO₂, —C(O)NR^{B}R^{C}, —C(O)R^{D}, —C(O)OR^{D}, or —S(O)ₓR^{D}, wherein each alkyl, alkylene, alkenyl, alkynyl, heteroalkyl, haloalkyl, cycloalkyl, heterocyclyl, aryl, and heteroaryl is optionally substituted with one or more R⁶; or two R¹ groups, together with the atoms to which they are attached, form a 3-7-membered cycloalkyl, heterocyclyl, aryl, or heteroaryl, wherein each cycloalkyl, heterocyclyl, aryl, and heteroaryl is optionally substituted with one or more R⁶; R² is hydrogen, halo, cyano, C₁-C₆-alkyl, C₂-C₆-alkenyl, C₂-C₆-alkynyl, C₁-C₆-heteroalkyl, C₁-C₆-haloalkyl, or —OR^{A}; each R³ is independently hydrogen, C₁-C₆-alkyl, or C₁-C₆-haloalkyl; each R⁴ is independently C₁-C₆-alkyl, C₁-C₆-heteroalkyl, C₁-C₆-haloalkyl, cycloalkyl, halo, cyano, oxo, —OR^{A}, —NR^{B}R^{C}, —C(O)R^{D}, or —C(O)OR^{D}; R⁵ is hydrogen, C₁-C₆-alkyl, C₂-C₆-alkenyl, C₂-C₆-alkynyl, C₁-C₆-heteroalkyl, C₁-C₆-haloalkyl, halo, cyano, or —OR^{A}; each R⁶ is independently C₁-C₆-alkyl, C₂-C₆-alkenyl, C₂-C₆-alkynyl, C₁-C₆-heteroalkyl, C₁-C₆-haloalkyl, cycloalkyl, heterocyclyl, aryl, heteroaryl, halo, cyano, oxo, —OR^{A}, —NR^{B}R^{C}, —NR^{B}C(O)R^{D}, —NO₂, —C(O)NR^{B}R^{C}, —C(O)R^{D}, —C(O)OR^{D}, or —S(O)ₓR^{D}, wherein each of alkyl, alkenyl, alkynyl, heteroalkyl, haloalkyl, cycloalkyl, heterocyclyl, aryl, and heteroaryl is optionally substituted with one or more R⁷; each R^{A} is independently hydrogen, C₁-C₆ alkyl, C₁-C₆ heteroalkyl, C₁-C₆ haloalkyl, cycloalkyl, heterocyclyl, aryl, heteroaryl, C₁-C₆ alkylene-aryl, C₁-C₆ alkylene-heteroaryl, —C(O)R^{D}, or —S(O)ₓR^{D}, wherein each alkyl, alkylene, heteroalkyl, haloalkyl, cycloalkyl, heterocyclyl, aryl, and heteroaryl is optionally substituted with one or more R⁸; each R^{B} and R^{C} is independently hydrogen, C₁-C₆ alkyl, C₁-C₆ heteroalkyl, C₁-C₆ haloalkyl, cycloalkyl, heterocyclyl, aryl, heteroaryl, C₁-C₆ alkylene-cycloalkyl, C₁-C₆ alkylene-heterocyclyl, —OR^{A}, wherein each alkyl, alkylene, heteroalkyl, haloalkyl, cycloalkyl, heterocyclyl, aryl, and heteroaryl is optionally substituted with one or more R⁸; or R^{B} and R^{C} together with the atom to which they are attached form a 3-7-membered heterocyclyl ring optionally substituted with one or more R⁸; each R^{D} is independently hydrogen, C₁-C₆ alkyl, C₂-C₆ alkenyl, C₂-C₆ alkynyl, C₁-C₆ heteroalkyl, C₁-C₆ haloalkyl, cycloalkyl, heterocyclyl, aryl, heteroaryl, C₁-C₆ alkylene-aryl, or C₁-C₆ alkylene-heteroaryl, wherein each alkyl, alkylene, alkenyl, alkynyl, heteroalkyl, haloalkyl, cycloalkyl, heterocyclyl, aryl, and heteroaryl is optionally substituted with one or more R⁸; each R⁷ is independently C₁-C₆ alkyl, C₁-C₆ heteroalkyl, C₁-C₆ haloalkyl, cycloalkyl, heterocyclyl, aryl, heteroaryl, halo, cyano, oxo, or —OR^{A}; each R⁸ is C₁-C₆-alkyl, halo, cyano, oxo, or —OR^{A1};
each R^{A1} is hydrogen or C₁-C₆-alkyl; and x is 0, 1, or 2.

In some embodiments, the compound of Formula (I) is a compound of Formula (I-b):

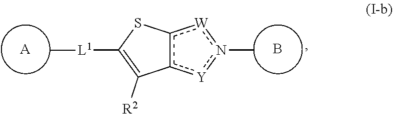

or a pharmaceutically acceptable salt, solvate, hydrate, tautomer, or stereoisomer thereof, wherein A and B are each independently cycloalkyl, heterocyclyl, aryl, or heteroaryl, each of which is optionally substituted with one or more R¹; L¹ is absent, C₁-C₆-alkylene, C₁-C₆-heteroalkylene, —O—, —C(O)—, —N(R³)—, —N(R³)C(O)—, or —C(O)N(R³)—, wherein each alkylene and heteroalkylene is optionally substituted with one or more R⁴; W and Y are each independently C, C(R⁵) or N; X is C or N; wherein at least one of W, X, and Y is N, and the dashed lines in the ring comprising W, X, and Y may be single or double bonds as valency permits; each R¹ is independently hydrogen, C₁-C₆-alkyl, C₂-C₆-alkenyl, C₂-C₆-alkynyl, C₁-C₆-heteroalkyl, C₁-C₆-haloalkyl, cycloalkyl, heterocyclyl, aryl, C₁-C₆ alkylene-aryl, C₁-C₆ alkenylene-aryl, C₁-C₆ alkylene-heteroaryl, heteroaryl, halo, cyano, oxo, —OR^{A}, —NR^{B}R^{C}, NR^{B}C(O)R^{D}, —NO₂, —C(O)NR^{B}R^{C}, —C(O)R^{D}, —C(O)OR^{D}, or —S(O)ₓR^{D}, wherein each alkyl, alkylene, alkenyl, alkynyl, heteroalkyl, haloalkyl, cycloalkyl, heterocyclyl, aryl, and heteroaryl is optionally substituted with one or more R⁶; or two R¹ groups, together with the atoms to which they are attached, form a 3-7-membered cycloalkyl, heterocyclyl, aryl, or heteroaryl, wherein each cycloalkyl, heterocyclyl, aryl, and heteroaryl is optionally substituted with one or more R⁶; R² is hydrogen, halo, cyano, C₁-C₆-alkyl, C₂-C₆-alkenyl, C₂-C₆-alkynyl, C₁-C₆-heteroalkyl, or C₁-C₆-haloalkyl; each R³ is independently hydrogen, C₁-C₆-alkyl, or C₁-C₆-haloalkyl; each R⁴ is independently C₁-C₆-alkyl, C₁-C₆-heteroalkyl, C₁-C₆-haloalkyl, cycloalkyl, halo, cyano, oxo, —OR^{A}, —NR^{B}R^{C}, —C(O)R^{D}, or —C(O)OR^{D}; R⁵ is hydrogen, C₁-C₆-alkyl, C₂-C₆-alkenyl, C₂-C₆-alkynyl, C₁-C₆-heteroalkyl, C₁-C₆-haloalkyl, halo, cyano, or —OR^{A}; each R⁶ is independently C₁-C₆-alkyl, C₂-C₆-alkenyl, C₂-C₆-alkynyl, C₁-C₆-heteroalkyl, C₁-C₆-haloalkyl, cycloalkyl, heterocyclyl, aryl, heteroaryl, halo, cyano, oxo, —OR^{A}, —NR^{B}R^{C}, —N^{B}C(O)R^{D}, —NO₂, —C(O)NR^{B}R^{C}, —C(O)R^{D}, —C(O)OR^{D}, or —S(O)ₓR^{D}, wherein each of alkyl, alkenyl, alkynyl, heteroalkyl, haloalkyl, cycloalkyl, heterocyclyl, aryl, and heteroaryl is optionally substituted with one or more R⁷; each R^{A} is independently hydrogen, C₁-C₆ alkyl, C₁-C₆ haloalkyl, aryl, heteroaryl, C₁-C₆ alkylene-aryl, C₁-C₆ alkylene-heteroaryl, —C(O)R^{D}, or —S(O)ₓR^{D}; each of R^{B} and R^{C} is independently hydrogen, C₁-C₆ alkyl, C₁-C₆ heteroalkyl, cycloalkyl, heterocyclyl, or —OR^{A}; or R^{B} and R^{C} together with the atom to which they are attached form a 3-7-membered heterocyclyl ring optionally substituted with one or more R⁸; each R is independently hydrogen, C₁-C₆ alkyl, C₂-C₆ alkenyl, C₂-C₆ alkynyl, C₁-C₆ heteroalkyl, C₁-C₆ haloalkyl, cycloalkyl, heterocyclyl, aryl, heteroaryl, C₁-C₆ alkylene-aryl, or C₁-C₆ alkylene-heteroaryl; each R⁷ is independently C₁-C₆ alkyl, C₁-C₆ heteroalkyl, C₁-C₆ haloalkyl, cycloalkyl, heterocyclyl, aryl, heteroaryl, halo, cyano, oxo, or —OR^{A}; each R⁸ is C₁-C₆-alkyl, halo, cyano, oxo, or —OR^{A1};
each R^{A1} is hydrogen or C₁-C₆-alkyl; and x is 0, 1, or 2.

In some embodiments, the compound of Formula (I) is a compound of Formula (I-c):

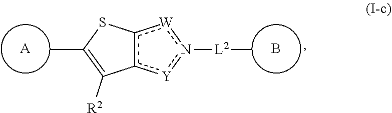

or a pharmaceutically acceptable salt, solvate, hydrate, tautomer, or stereoisomer thereof, wherein A and B are each independently cycloalkyl, heterocyclyl, aryl, or heteroaryl, each of which is optionally substituted with one or more R¹; L² is absent, C₁-C₆-alkylene, C₁-C₆-heteroalkylene, —O—, —C(O)—, —N(R³)—, —N(R³)C(O)—, or —C(O)N(R³)—, wherein each alkylene and heteroalkylene is optionally substituted with one or more R⁴; W and Y are each independently C, C(R⁵) or N; X is C or N; wherein at least one of W, X, and Y is N, and the dashed lines in the ring comprising W, X, and Y may be single or double bonds as valency permits; each R¹ is independently hydrogen, C₁-C₆-alkyl, C₂-C₆-alkenyl, C₂-C₆-alkynyl, C₁-C₆-heteroalkyl, C₁-C₆-haloalkyl, cycloalkyl, heterocyclyl, aryl, C₁-C₆ alkylene-aryl, C₁-C₆ alkenylene-aryl, C₁-C₆ alkylene-heteroaryl, heteroaryl, halo, cyano, oxo, —OR^{A}, —NR^{B}R^{C}, —N^{B}C(O)R^{D}, —NO₂, —C(O)NR^{B}R^{C}, —C(O)R^{D}, —C(O)OR^{D}, or —S(O)ₓR^{D}, wherein each alkyl, alkylene, alkenyl, alkynyl, heteroalkyl, haloalkyl, cycloalkyl, heterocyclyl, aryl, and heteroaryl is optionally substituted with one or more R⁶; or two R¹ groups, together with the atoms to which they are attached, form a 3-7-membered cycloalkyl, heterocyclyl, aryl, or heteroaryl, wherein each cycloalkyl, heterocyclyl, aryl, and heteroaryl is optionally substituted with one or more R⁶; R² is hydrogen, halo, cyano, C₁-C₆-alkyl, C₂-C₆-alkenyl, C₂-C₆-alkynyl, C₁-C₆-heteroalkyl, or C₁-C₆-haloalkyl; each R³ is independently hydrogen, C₁-C₆-alkyl, or C₁-C₆-haloalkyl; each R⁴ is independently C₁-C₆-alkyl, C₁-C₆-heteroalkyl, C₁-C₆-haloalkyl, cycloalkyl, halo, cyano, oxo, —OR^{A}, —NR^{B}R^{C}, —C(O)R^{D}, or —C(O)OR^{D}; R⁵ is hydrogen or C₁-C₆-alkyl, C₂-C₆-alkenyl, C₂-C₆-alkynyl, C₁-C₆-heteroalkyl, C₁-C₆-haloalkyl, halo, cyano, or —OR^{A}; each R⁶ is independently C₁-C₆-alkyl, C₂-C₆-alkenyl, C₂-C₆-alkynyl, C₁-C₆-heteroalkyl, C₁-C₆-haloalkyl, cycloalkyl, heterocyclyl, aryl, heteroaryl, halo, cyano, oxo, —OR^{A}, —NR^{B}R^{C}, —N^{B}C(O)R^{D}, —NO₂, —C(O)NR^{B}R^{C}, —C(O)R^{D}, —C(O)OR^{D}, or —S(O)ₓR^{D}, wherein each of alkyl, alkenyl, alkynyl, heteroalkyl, haloalkyl, cycloalkyl, heterocyclyl, aryl, and heteroaryl is optionally substituted with one or more R⁷; each R^{A} is independently hydrogen, C₁-C₆ alkyl, C₁-C₆ haloalkyl, aryl, heteroaryl, C₁-C₆ alkylene-aryl, C₁-C₆ alkylene-heteroaryl, —C(O)R^{D}, or —S(O)ₓR^{D}; each of R^{B} and R^{C} is independently hydrogen, C₁-C₆ alkyl, C₁-C₆ heteroalkyl, cycloalkyl, heterocyclyl, or —OR^{A}; or R^{B} and R^{C} together with the atom to which they are attached form a 3-7-membered heterocyclyl ring optionally substituted with one or more R⁸; each R is independently hydrogen, C₁-C₆ alkyl, C₂-C₆ alkenyl, C₂-C₆ alkynyl, C₁-C₆ heteroalkyl, C₁-C₆ haloalkyl, cycloalkyl, heterocyclyl, aryl, heteroaryl, C₁-C₆ alkylene-aryl, or C₁-C₆ alkylene-heteroaryl; each R⁷ is independently C₁-C₆ alkyl, C₁-C₆ heteroalkyl, C₁-C₆ haloalkyl, cycloalkyl, heterocyclyl, aryl, heteroaryl, halo, cyano, oxo, or —OR^{A}; each R⁸ is C₁-C₆-alkyl, halo, cyano, oxo, or —OR^{A1};
each R^{A1} is hydrogen or C₁-C₆-alkyl; and x is 0, 1, or 2.

In some embodiments the compound of Formula (I) is a compound of Formula (I-d):

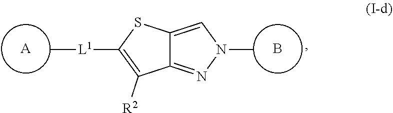

or a pharmaceutically acceptable salt, solvate, hydrate, tautomer, or stereoisomer thereof, wherein A and B are each independently cycloalkyl, heterocyclyl, aryl, or heteroaryl, each of which is optionally substituted with one or more R¹; L¹ is absent, C₁-C₆-alkylene, C₁-C₆-heteroalkylene, —O—, —C(O)—, —N(R³)—, —N(R³)C(O)—, or —C(O)N(R³)—, wherein each alkylene and heteroalkylene is optionally substituted with one or more R⁴; each R¹ is independently hydrogen, C₁-C₆-alkyl, C₂-C₆-alkenyl, C₂-C₆-alkynyl, C₁-C₆-heteroalkyl, C₁-C₆-haloalkyl, cycloalkyl, heterocyclyl, aryl, C₁-C₆ alkylene-aryl, C₁-C₆ alkenylene-aryl, C₁-C₆ alkylene-heteroaryl, heteroaryl, halo, cyano, oxo, —OR^{A}, —NR^{B}R^{C}, —NR^{B}C(O)R^{D}, —NO₂, —C(O)NR^{B}R^{C}, —C(O)R^{D}, —C(O)OR^{D}, or —S(O)ₓR^{D}, wherein each alkyl, alkylene, alkenyl, alkynyl, heteroalkyl, haloalkyl, cycloalkyl, heterocyclyl, aryl, and heteroaryl is optionally substituted with one or more R⁶; or two R¹ groups, together with the atoms to which they are attached, form a 3-7-membered cycloalkyl, heterocyclyl, aryl, or heteroaryl, wherein each cycloalkyl, heterocyclyl, aryl, and heteroaryl is optionally substituted with one or more R⁶; R² is hydrogen, halo, cyano, C₁-C₆-alkyl, C₂-C₆-alkenyl, C₂-C₆-alkynyl, C₁-C₆-heteroalkyl, or C₁-C₆-haloalkyl; each R³ is independently hydrogen, C₁-C₆-alkyl, or C₁-C₆-haloalkyl; each R⁴ is independently C₁-C₆-alkyl, C₁-C₆-heteroalkyl, C₁-C₆-haloalkyl, cycloalkyl, halo, cyano, oxo, —OR^{A}, —NR^{B}R^{C}, —C(O)R^{D}, or —C(O)OR^{D}; each R⁶ is independently C₁-C₆-alkyl, C₂-C₆-alkenyl, C₂-C₆-alkynyl, C₁-C₆-heteroalkyl, C₁-C₆-haloalkyl, cycloalkyl, heterocyclyl, aryl, heteroaryl, halo, cyano, oxo, —OR^{A}, —NR^{B}R^{C}, NR^{B}C(O)R^{D}, —NO₂, —C(O)NR^{B}R^{C}, —C(O)R^{D}, —C(O)OR^{D}, or —S(O)ₓR^{D}, wherein each of alkyl, alkenyl, alkynyl, heteroalkyl, haloalkyl, cycloalkyl, heterocyclyl, aryl, and heteroaryl is optionally substituted with one or more R⁷; each R^{A} is independently hydrogen, C₁-C₆ alkyl, C₁-C₆ haloalkyl, aryl, heteroaryl, C₁-C₆ alkylene-aryl, C₁-C₆ alkylene-heteroaryl, —C(O)R^{D}, or —S(O)ₓR^{D}; each of R^{B} and R^{C} is independently hydrogen, C₁-C₆ alkyl, C₁-C₆ heteroalkyl, cycloalkyl, heterocyclyl, or —OR^{A}; or R^{B} and R^{C} together with the atom to which they are attached form a 3-7-membered heterocyclyl ring optionally substituted with one or more R⁸; each R^{D} is independently hydrogen, C₁-C₆ alkyl, C₂-C₆ alkenyl, C₂-C₆ alkynyl, C₁-C₆ heteroalkyl, C₁-C₆ haloalkyl, cycloalkyl, heterocyclyl, aryl, heteroaryl, C₁-C₆ alkylene-aryl, or C₁-C₆ alkylene-heteroaryl; each R⁷ is independently C₁-C₆ alkyl, C₁-C₆ heteroalkyl, C₁-C₆ haloalkyl, cycloalkyl, heterocyclyl, aryl, heteroaryl, halo, cyano, oxo, or —OR^{A}; each R⁸ is C₁-C₆-alkyl, halo, cyano, oxo, or —OR^{A1}; each R^{A1} is hydrogen or C₁-C₆-alkyl; and x is 0, 1, or 2.

In some embodiments, the compound of Formula (I) is a compound of Formula (I-e):

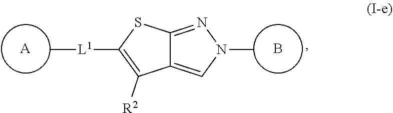

or a pharmaceutically acceptable salt, solvate, hydrate, tautomer, or stereoisomer thereof, wherein A and B are each independently cycloalkyl, heterocyclyl, aryl, or heteroaryl, each of which is optionally substituted with one or more R¹; L¹ is absent, C₁-C₆-alkylene, C₁-C₆-heteroalkylene, —O—, —C(O)—, —N(R³)—, —N(R³)C(O)—, or —C(O)N(R³)—, wherein each alkylene and heteroalkylene is optionally substituted with one or more R⁴; each R¹ is independently hydrogen, C₁-C₆-alkyl, C₂-C₆-alkenyl, C₂-C₆-alkynyl, C₁-C₆-heteroalkyl, C₁-C₆-haloalkyl, cycloalkyl, heterocyclyl, aryl, C₁-C₆ alkylene-aryl, C₁-C₆ alkenylene-aryl, C₁-C₆ alkylene-heteroaryl, heteroaryl, halo, cyano, oxo, —OR^{A}, —NR^{B}R^{C}, —NR^{B}C(O)R^{D}, —NO₂, —C(O)NR^{B}R^{C}, —C(O)R^{D}, —C(O)OR^{D}, or —S(O)ₓR^{D}, wherein each alkyl, alkylene, alkenyl, alkynyl, heteroalkyl, haloalkyl, cycloalkyl, heterocyclyl, aryl, and heteroaryl is optionally substituted with one or more R⁶; or two R¹ groups, together with the atoms to which they are attached, form a 3-7-membered cycloalkyl, heterocyclyl, aryl, or heteroaryl, wherein each cycloalkyl, heterocyclyl, aryl, and heteroaryl is optionally substituted with one or more R⁶; R² is hydrogen, halo, cyano, C₁-C₆-alkyl, C₂-C₆-alkenyl, C₂-C₆-alkynyl, C₁-C₆-heteroalkyl, or C₁-C₆-haloalkyl; each R³ is independently hydrogen, C₁-C₆-alkyl, or C₁-C₆-haloalkyl; each R⁴ is independently C₁-C₆-alkyl, C₁-C₆-heteroalkyl, C₁-C₆-haloalkyl, cycloalkyl, halo, cyano, oxo, —OR^{A}, —NR^{B}R^{C}, —C(O)R^{D}, or —C(O)OR^{D}; each R⁶ is independently C₁-C₆-alkyl, C₂-C₆-alkenyl, C₂-C₆-alkynyl, C₁-C₆-heteroalkyl, C₁-C₆-haloalkyl, cycloalkyl, heterocyclyl, aryl, heteroaryl, halo, cyano, oxo, —OR^{A}, —NR^{B}R^{C}, NR^{B}C(O)R^{D}, —NO₂, —C(O)NR^{B}R^{C}, —C(O)R^{D}, —C(O)OR^{D}, or —S(O)ₓR^{D}, wherein each of alkyl, alkenyl, alkynyl, heteroalkyl, haloalkyl, cycloalkyl, heterocyclyl, aryl, and heteroaryl is optionally substituted with one or more R⁷; each R^{A} is independently hydrogen, C₁-C₆ alkyl, C₁-C₆ haloalkyl, aryl, heteroaryl, C₁-C₆ alkylene-aryl, C₁-C₆ alkylene-heteroaryl, —C(O)R^{D}, or —S(O)ₓR^{D}; each of R^{B} and R^{C} is independently hydrogen, C₁-C₆ alkyl, C₁-C₆ heteroalkyl, cycloalkyl, heterocyclyl, or —OR^{A}; or R^{B} and R^{C} together with the atom to which they are attached form a 3-7-membered heterocyclyl ring optionally substituted with one or more R⁸; each R^{D} is independently hydrogen, C₁-C₆ alkyl, C₂-C₆ alkenyl, C₂-C₆ alkynyl, C₁-C₆ heteroalkyl, C₁-C₆ haloalkyl, cycloalkyl, heterocyclyl, aryl, heteroaryl, C₁-C₆ alkylene-aryl, or C₁-C₆ alkylene-heteroaryl; each R⁷ is independently C₁-C₆ alkyl, C₁-C₆ heteroalkyl, C₁-C₆ haloalkyl, cycloalkyl, heterocyclyl, aryl, heteroaryl, halo, cyano, oxo, or —OR^{A}; each R⁸ is C₁-C₆-alkyl, halo, cyano, oxo, or —OR^{A1}; each R^{A1} is hydrogen or C₁-C₆-alkyl; and x is 0, 1, or 2.

In some embodiment, the compound of Formula (I) is a compound of Formula (I-f):

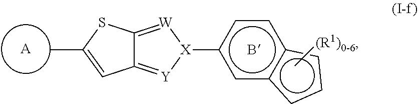

or a pharmaceutically acceptable salt, solvate, hydrate, tautomer, or stereoisomer thereof, wherein A is cycloalkyl, heterocyclyl, aryl, or heteroaryl, each of which is optionally substituted with one or more R¹; B′ is bicyclic heteroaryl; W and Y are each independently C, C(R⁵) or N; X is C or N; wherein at least one of W, X, and Y is N; each R¹ is independently hydrogen, C₁-C₆-alkyl, C₂-C₆-alkenyl, C₂-C₆-alkynyl, C₁-C₆-heteroalkyl, C₁-C₆-haloalkyl, cycloalkyl, heterocyclyl, aryl, C₁-C₆ alkylene-aryl, C₁-C₆ alkenylene-aryl, C₁-C₆ alkylene-heteroaryl, heteroaryl, halo, cyano, oxo, —OR^{A}, —NR^{B}R^{C}, —N^{B}C(O)R^{D}, —NO₂, —C(O)NR^{B}R^{C}, —C(O)R^{D}, —C(O)OR^{D}, or —S(O)ₓR^{D}, wherein each alkyl, alkylene, alkenyl, alkynyl, heteroalkyl, haloalkyl, cycloalkyl, heterocyclyl, aryl, and heteroaryl is optionally substituted with one or more R⁶; or two R¹ groups, together with the atoms to which they are attached, form a 3-7-membered cycloalkyl, heterocyclyl, aryl, or heteroaryl, wherein each cycloalkyl, heterocyclyl, aryl, and heteroaryl is optionally substituted with one or more R⁶; each R⁶ is independently C₁-C₆-alkyl, C₂-C₆-alkenyl, C₂-C₆-alkynyl, C₁-C₆-heteroalkyl, C₁-C₆-haloalkyl, cycloalkyl, heterocyclyl, aryl, heteroaryl, halo, cyano, oxo, —OR^{A}, —NR^{B}R^{C}, —N^{B}C(O)R^{D}, —NO₂, —C(O)NR^{B}R^{C}, —C(O)R^{D}, —C(O)OR^{D}, or —S(O)ₓR^{D}, wherein each of alkyl, alkenyl, alkynyl, heteroalkyl, haloalkyl, cycloalkyl, heterocyclyl, aryl, and heteroaryl is optionally substituted with one or more R⁷; each R^{A} is independently hydrogen, C₁-C₆ alkyl, C₁-C₆ haloalkyl, aryl, heteroaryl, C₁-C₆ alkylene-aryl, C₁-C₆ alkylene-heteroaryl, —C(O)R^{D}, or —S(O)ₓR^{D}; each of R^{B} and R^{C} is independently hydrogen, C₁-C₆ alkyl, C₁-C₆ heteroalkyl, cycloalkyl, heterocyclyl, or —OR^{A}; or R^{B} and R^{C} together with the atom to which they are attached form a 3-7-membered heterocyclyl ring optionally substituted with one or more R⁸; each R^{D} is independently hydrogen, C₁-C₆ alkyl, C₂-C₆ alkenyl, C₂-C₆ alkynyl, C₁-C₆ heteroalkyl, C₁-C₆ haloalkyl, cycloalkyl, heterocyclyl, aryl, heteroaryl, C₁-C₆ alkylene-aryl, or C₁-C₆ alkylene-heteroaryl; each R⁷ is independently C₁-C₆ alkyl, C₁-C₆ heteroalkyl, C₁-C₆ haloalkyl, cycloalkyl, heterocyclyl, aryl, heteroaryl, halo, cyano, oxo, or —OR^{A}; each R⁸ is C₁-C₆-alkyl, halo, cyano, oxo, or —OR^{A1}; each R^{A1} is hydrogen or C₁-C₆-alkyl; and x is 0, 1, or 2.

In some embodiments, the compound of Formula (I) is a compound of Formula (I-g):

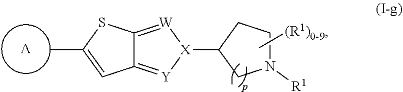

or a pharmaceutically acceptable salt, solvate, hydrate, tautomer, or stereoisomer thereof, wherein A is cycloalkyl, heterocyclyl, aryl, or heteroaryl, each of which is optionally substituted with one or more R¹; W and Y are each independently C, C(R⁵) or N; X is C or N; wherein at least one of W, X, and Y is N; each R¹ is independently hydrogen, C₁-C₆-alkyl, C₂-C₆-alkenyl, C₂-C₆-alkynyl, C₁-C₆-heteroalkyl, C₁-C₆-haloalkyl, cycloalkyl, heterocyclyl, aryl, C₁-C₆ alkylene-aryl, C₁-C₆ alkenylene-aryl, C₁-C₆ alkylene-heteroaryl, heteroaryl, halo, cyano, oxo, —OR^{A}, —NR^{B}R^{C}, —NR^{B}C(O)R^{D}, —NO₂, —C(O)NR^{B}R^{C}, —C(O)R^{D}, —C(O)OR^{D}, or —S(O)ₓR^{D}, wherein each alkyl, alkylene, alkenyl, alkynyl, heteroalkyl, haloalkyl, cycloalkyl, heterocyclyl, aryl, and heteroaryl is optionally substituted with one or more R⁶; or two R¹ groups, together with the atoms to which they are attached, form a 3-7-membered cycloalkyl, heterocyclyl, aryl, or heteroaryl, wherein each cycloalkyl, heterocyclyl, aryl, and heteroaryl is optionally substituted with one or more R⁶ each R⁶ is independently C₁-C₆-alkyl, C₂-C₆-alkenyl, C₂-C₆-alkynyl, C₁-C₆-heteroalkyl, C₁-C₆-haloalkyl, cycloalkyl, heterocyclyl, aryl, heteroaryl, halo, cyano, oxo, —OR^{A}, —NR^{B}R^{C}, —NR^{B}C(O)R^{D}, —NO₂, —C(O)NR^{B}R^{C}, —C(O)R^{D}, —C(O)OR^{D}, or —S(O)ₓR^{D}, wherein each of alkyl, alkenyl, alkynyl, heteroalkyl, haloalkyl, cycloalkyl, heterocyclyl, aryl, and heteroaryl is optionally substituted with one or more R⁷; each R^{A} is independently hydrogen, C₁-C₆ alkyl, C₁-C₆ haloalkyl, aryl, heteroaryl, C₁-C₆ alkylene-aryl, C₁-C₆ alkylene-heteroaryl, —C(O)R^{D}, or —S(O)ₓR^{D}; each of R^{B} and R^{C} is independently hydrogen, C₁-C₆ alkyl, C₁-C₆ heteroalkyl, cycloalkyl, heterocyclyl, or —OR^{A}; or R^{B} and R^{C} together with the atom to which they are attached form a 3-7-membered heterocyclyl ring optionally substituted with one or more R⁸; each R^{D} is independently hydrogen, C₁-C₆ alkyl, C₂-C₆ alkenyl, C₂-C₆ alkynyl, C₁-C₆ heteroalkyl, C₁-C₆ haloalkyl, cycloalkyl, heterocyclyl, aryl, heteroaryl, C₁-C₆ alkylene-aryl, or C₁-C₆ alkylene-heteroaryl; each R⁷ is independently C₁-C₆ alkyl, C₁-C₆ heteroalkyl, C₁-C₆ haloalkyl, cycloalkyl, heterocyclyl, aryl, heteroaryl, halo, cyano, oxo, or —OR^{A}; each R⁸ is C₁-C₆-alkyl, halo, cyano, oxo, or —OR^{A1}; each R^{A1} is hydrogen or C₁-C₆-alkyl; p is 0, 1, 2, or 3; and x is 0, 1, or 2.

In some embodiments, the compound of Formula (I) is a compound of Formula (I-h):

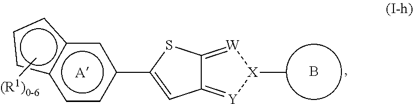

or a pharmaceutically acceptable salt, solvate, hydrate, tautomer, or stereoisomer thereof, wherein A′ is bicyclic heteroaryl; B is cycloalkyl, heterocyclyl, aryl, or heteroaryl, each of which is optionally substituted with one or more R¹; W and Y are each independently C, C(R⁵) or N; X is C or N; wherein at least one of W, X, and Y is N; each R¹ is independently hydrogen, C₁-C₆-alkyl, C₂-C₆-alkenyl, C₂-C₆-alkynyl, C₁-C₆-heteroalkyl, C₁-C₆-haloalkyl, cycloalkyl, heterocyclyl, aryl, C₁-C₆ alkylene-aryl, C₁-C₆ alkenylene-aryl, C₁-C₆ alkylene-heteroaryl, heteroaryl, halo, cyano, oxo, —OR^{A}, —NR^{B}R^{C}, —NR^{B}C(O)R^{D}, —NO₂, —C(O)NR^{B}R^{C}, —C(O)R^{D}, —C(O)OR^{D}, or —S(O)ₓR^{D}, wherein each alkyl, alkylene, alkenyl, alkynyl, heteroalkyl, haloalkyl, cycloalkyl, heterocyclyl, aryl, and heteroaryl is optionally substituted with one or more R⁶; or two R¹ groups, together with the atoms to which they are attached, form a 3-7-membered cycloalkyl, heterocyclyl, aryl, or heteroaryl, wherein each cycloalkyl, heterocyclyl, aryl, and heteroaryl is optionally substituted with one or more R⁶; each R⁶ is independently C₁-C₆-alkyl, C₂-C₆-alkenyl, C₂-C₆-alkynyl, C₁-C₆-heteroalkyl, C₁-C₆-haloalkyl, cycloalkyl, heterocyclyl, aryl, heteroaryl, halo, cyano, oxo, —OR^{A}, —NR^{B}R^{C}, —NR^{B}C(O)R^{D}, —NO₂, —C(O)NR^{B}R^{C}, —C(O)R^{D}, —C(O)OR^{D}, or —S(O)ₓR^{D}, wherein each of alkyl, alkenyl, alkynyl, heteroalkyl, haloalkyl, cycloalkyl, heterocyclyl, aryl, and heteroaryl is optionally substituted with one or more R⁷; each R^{A} is independently hydrogen, C₁-C₆ alkyl, C₁-C₆ haloalkyl, aryl, heteroaryl, C₁-C₆ alkylene-aryl, C₁-C₆ alkylene-heteroaryl, —C(O)R^{D}, or —S(O)ₓR^{D}; each of R^{B} and R^{C} is independently hydrogen, C₁-C₆ alkyl, C₁-C₆ heteroalkyl, cycloalkyl, heterocyclyl, or —OR^{A}; or R^{B} and R^{C} together with the atom to which they are attached form a 3-7-membered heterocyclyl ring optionally substituted with one or more R⁸; each R^{D} is independently hydrogen, C₁-C₆ alkyl, C₂-C₆ alkenyl, C₂-C₆ alkynyl, C₁-C₆ heteroalkyl, C₁-C₆ haloalkyl, cycloalkyl, heterocyclyl, aryl, heteroaryl, C₁-C₆ alkylene-aryl, or C₁-C₆ alkylene-heteroaryl; each R⁷ is independently C₁-C₆ alkyl, C₁-C₆ heteroalkyl, C₁-C₆ haloalkyl, cycloalkyl, heterocyclyl, aryl, heteroaryl, halo, cyano, oxo, or —OR^{A}; each R⁸ is C₁-C₆-alkyl, halo, cyano, oxo, or —OR^{A1}; each R^{A1} is hydrogen or C₁-C₆-alkyl; and x is 0, 1, or 2.

In some embodiments, the compound of Formula (I) is a compound of Formula (I-i):

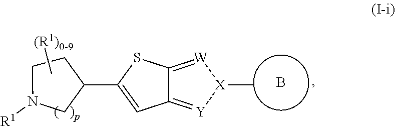

or a pharmaceutically acceptable salt, solvate, hydrate, tautomer, or stereoisomer thereof, wherein B is cycloalkyl, heterocyclyl, aryl, or heteroaryl, each of which is optionally substituted with one or more R¹; W and Y are each independently C, C(R⁵) or N; X is C or N; wherein at least one of W, X, and Y is N; each R¹ is independently hydrogen, C₁-C₆-alkyl, C₂-C₆-alkenyl, C₂-C₆-alkynyl, C₁-C₆-heteroalkyl, C₁-C₆-haloalkyl, cycloalkyl, heterocyclyl, aryl, C₁-C₆ alkylene-aryl, C₁-C₆ alkenylene-aryl, C₁-C₆ alkylene-heteroaryl, heteroaryl, halo, cyano, oxo, —OR^{A}, —NR^{B}R^{C}, —NR^{B}C(O)R^{D}, —NO₂, —C(O)NR^{B}R^{C}, —C(O)R^{D}, —C(O)OR^{D}, or —S(O)ₓR^{D}, wherein each alkyl, alkylene, alkenyl, alkynyl, heteroalkyl, haloalkyl, cycloalkyl, heterocyclyl, aryl, and heteroaryl is optionally substituted with one or more R⁶; or two R¹ groups, together with the atoms to which they are attached, form a 3-7-membered cycloalkyl, heterocyclyl, aryl, or heteroaryl, wherein each cycloalkyl, heterocyclyl, aryl, and heteroaryl is optionally substituted with one or more R⁶ each R⁶ is independently C₁-C₆-alkyl, C₂-C₆-alkenyl, C₂-C₆-alkynyl, C₁-C₆-heteroalkyl, C₁-C₆-haloalkyl, cycloalkyl, heterocyclyl, aryl, heteroaryl, halo, cyano, oxo, —OR^{A}, —NR^{B}R^{C}, —NR^{B}C(O)R^{D}, —NO₂, —C(O)NR^{B}R^{C}, —C(O)R^{D}, —C(O)OR^{D}, or —S(O)ₓR^{D}, wherein each of alkyl, alkenyl, alkynyl, heteroalkyl, haloalkyl, cycloalkyl, heterocyclyl, aryl, and heteroaryl is optionally substituted with one or more R⁷; each R^{A} is independently hydrogen, C₁-C₆ alkyl, C₁-C₆ haloalkyl, aryl, heteroaryl, C₁-C₆ alkylene-aryl, C₁-C₆ alkylene-heteroaryl, —C(O)R^{D}, or —S(O)ₓR^{D}; each of R^{B} and R^{C} is independently hydrogen, C₁-C₆ alkyl, C₁-C₆ heteroalkyl, cycloalkyl, heterocyclyl, or —OR^{A}; or R^{B} and R^{C} together with the atom to which they are attached form a 3-7-membered heterocyclyl ring optionally substituted with one or more R⁸; each R^{D} is independently hydrogen, C₁-C₆ alkyl, C₂-C₆ alkenyl, C₂-C₆ alkynyl, C₁-C₆ heteroalkyl, C₁-C₆ haloalkyl, cycloalkyl, heterocyclyl, aryl, heteroaryl, C₁-C₆ alkylene-aryl, or C₁-C₆ alkylene-heteroaryl; each R⁷ is independently C₁-C₆ alkyl, C₁-C₆ heteroalkyl, C₁-C₆ haloalkyl, cycloalkyl, heterocyclyl, aryl, heteroaryl, halo, cyano, oxo, or —OR^{A}; each R⁸ is C₁-C₆-alkyl, halo, cyano, oxo, or —OR^{A1}; each R^{A1} is hydrogen or C₁-C₆-alkyl; p is 0, 1, 2, or 3; and x is 0, 1, or 2.

In some embodiments, the compound of Formula (I) is a compound of Formula (I-j):

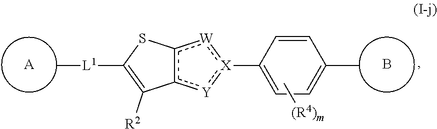

or a pharmaceutically acceptable salt, solvate, hydrate, tautomer, or stereoisomer thereof, wherein A and B are each independently cycloalkyl, heterocyclyl, aryl, or heteroaryl, each of which is optionally substituted with one or more R¹; L¹ is absent, C₁-C₆-alkylene, C₁-C₆-heteroalkylene, —O—, —C(O)—, —N(R³)—, —N(R³)C(O)—, or —C(O)N(R³)—, wherein each alkylene and heteroalkylene is optionally substituted with one or more R⁴; W and Y are each independently C(R⁵) or N; X is C or N; wherein at least one of W, X, and Y is N, and the dashed lines in the ring comprising W, X, and Y may be single or double bonds as valency permits; each R¹ is independently hydrogen, C₁-C₆-alkyl, C₂-C₆-alkenyl, C₂-C₆-alkynyl, C₁-C₆-heteroalkyl, C₁-C₆-haloalkyl, cycloalkyl, heterocyclyl, aryl, C₁-C₆ alkylene-aryl, C₁-C₆ alkenylene-aryl, C₁-C₆ alkylene-heteroaryl, heteroaryl, halo, cyano, oxo, —OR^{A}, —NR^{B}R^{C}, —NR^{B}C(O)R^{D}, —NO₂, —C(O)NR^{B}R^{C}, —C(O)R^{D}, —C(O)OR^{D}, or —S(O)ₓR^{D}, wherein each alkyl, alkylene, alkenyl, alkynyl, heteroalkyl, haloalkyl, cycloalkyl, heterocyclyl, aryl, and heteroaryl is optionally substituted with one or more R⁶; or two R¹ groups, together with the atoms to which they are attached, form a 3-7-membered cycloalkyl, heterocyclyl, aryl, or heteroaryl, wherein each cycloalkyl, heterocyclyl, aryl, and heteroaryl is optionally substituted with one or more R⁶; R² is hydrogen, halo, cyano, C₁-C₆-alkyl, C₂-C₆-alkenyl, C₂-C₆-alkynyl, C₁-C₆-heteroalkyl, or C₁-C₆-haloalkyl; each R³ is independently hydrogen, C₁-C₆-alkyl, or C₁-C₆-haloalkyl; each R⁴ is independently C₁-C₆-alkyl, C₁-C₆-heteroalkyl, C₁-C₆-haloalkyl, cycloalkyl, halo, cyano, oxo, —OR^{A}, —NR^{B}R^{C}, —C(O)R^{D}, or —C(O)OR^{D}; R⁵ is hydrogen or C₁-C₆-alkyl, C₂-C₆-alkenyl, C₂-C₆-alkynyl, C₁-C₆-heteroalkyl, C₁-C₆-haloalkyl, halo, cyano, or —OR^{A}; each R⁶ is independently C₁-C₆-alkyl, C₂-C₆-alkenyl, C₂-C₆-alkynyl, C₁-C₆-heteroalkyl, C₁-C₆-haloalkyl, cycloalkyl, heterocyclyl, aryl, heteroaryl, halo, cyano, oxo, —OR^{A}, —NR^{B}R^{C}, NR^{B}C(O)R^{D}, —NO₂, —C(O)NR^{B}R^{C}, —C(O)R^{D}, —C(O)OR^{D}, or —S(O)ₓR^{D}, wherein each of alkyl, alkenyl, alkynyl, heteroalkyl, haloalkyl, cycloalkyl, heterocyclyl, aryl, and heteroaryl is optionally substituted with one or more R⁷; each R^{A} is independently hydrogen, C₁-C₆ alkyl, C₁-C₆ haloalkyl, aryl, heteroaryl, C₁-C₆ alkylene-aryl, C₁-C₆ alkylene-heteroaryl, —C(O)R^{D}, or —S(O)ₓR^{D}; each of R^{B} and R^{C} is independently hydrogen, C₁-C₆ alkyl, C₁-C₆ heteroalkyl, cycloalkyl, heterocyclyl, or —OR^{A}; or R^{B} and R^{C} together with the atom to which they are attached form a 3-7-membered heterocyclyl ring optionally substituted with one or more R⁸; each R^{D} is independently hydrogen, C₁-C₆ alkyl, C₂-C₆ alkenyl, C₂-C₆ alkynyl, C₁-C₆ heteroalkyl, C₁-C₆ haloalkyl, cycloalkyl, heterocyclyl, aryl, heteroaryl, C₁-C₆ alkylene-aryl, or C₁-C₆ alkylene-heteroaryl; each R⁷ is independently C₁-C₆ alkyl, C₁-C₆ heteroalkyl, C₁-C₆ haloalkyl, cycloalkyl, heterocyclyl, aryl, heteroaryl, halo, cyano, oxo, or —OR^{A}; each R⁸ is C₁-C₆-alkyl, halo, cyano, oxo, or —OR^{A1}; each R^{A1} is hydrogen or C₁-C₆-alkyl; m is 0, 1, 2, 3, or 4; and x is 0, 1, or 2.

In some embodiments, the compound of Formula (I) is a compound of Formula (I-k):

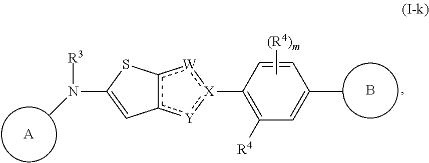

or a pharmaceutically acceptable salt, solvate, hydrate, tautomer, or stereoisomer thereof, wherein A and B are each independently cycloalkyl, heterocyclyl, aryl, or heteroaryl, each of which is optionally substituted with one or more R¹; W and Y are each independently C(R⁵) or N; X is C or N; wherein at least one of W, X, and Y is N, and the dashed lines in the ring comprising W, X, and Y may be single or double bonds as valency permits; each R¹ is independently hydrogen, C₁-C₆-alkyl, C₂-C₆-alkenyl, C₂-C₆-alkynyl, C₁-C₆-heteroalkyl, C₁-C₆-haloalkyl, cycloalkyl, heterocyclyl, aryl, C₁-C₆ alkylene-aryl, C₁-C₆ alkenylene-aryl, C₁-C₆ alkylene-heteroaryl, heteroaryl, halo, cyano, oxo, —OR^{A}, —NR^{B}R^{C}, —N^{B}C(O)R^{D}, —NO₂, —C(O)NR^{B}R^{C}, —C(O)R^{D}, —C(O)OR^{D}, or —S(O)ₓR^{D}, wherein each alkyl, alkylene, alkenyl, alkynyl, heteroalkyl, haloalkyl, cycloalkyl, heterocyclyl, aryl, and heteroaryl is optionally substituted with one or more R⁶; or two R¹ groups, together with the atoms to which they are attached, form a 3-7-membered cycloalkyl, heterocyclyl, aryl, or heteroaryl, wherein each cycloalkyl, heterocyclyl, aryl, and heteroaryl is optionally substituted with one or more R⁶; each R³ is independently hydrogen, C₁-C₆-alkyl, or C₁-C₆-haloalkyl; each R⁴ is independently C₁-C₆-alkyl, C₁-C₆-heteroalkyl, C₁-C₆-haloalkyl, cycloalkyl, halo, cyano, oxo, —OR^{A}, —NR^{B}R^{C}, —C(O)R^{D}, or —C(O)OR^{D}; R⁵ is hydrogen or C₁-C₆-alkyl, C₂-C₆-alkenyl, C₂-C₆-alkynyl, C₁-C₆-heteroalkyl, C₁-C₆-haloalkyl, halo, cyano, or —OR^{A}; each R⁶ is independently C₁-C₆-alkyl, C₂-C₆-alkenyl, C₂-C₆-alkynyl, C₁-C₆-heteroalkyl, C₁-C₆-haloalkyl, cycloalkyl, heterocyclyl, aryl, heteroaryl, halo, cyano, oxo, —OR^{A}, —NR^{B}R^{C}, NR^{B}C(O)R^{D}, —NO₂, —C(O)NR^{B}R^{C}, —C(O)R^{D}, —C(O)OR^{D}, or —S(O)ₓR^{D}, wherein each of alkyl, alkenyl, alkynyl, heteroalkyl, haloalkyl, cycloalkyl, heterocyclyl, aryl, and heteroaryl is optionally substituted with one or more R⁷; each R^{A} is independently hydrogen, C₁-C₆ alkyl, C₁-C₆ haloalkyl, aryl, heteroaryl, C₁-C₆ alkylene-aryl, C₁-C₆ alkylene-heteroaryl, —C(O)R^{D}, or —S(O)ₓR^{D}; each of R^{B} and R^{C} is independently hydrogen, C₁-C₆ alkyl, C₁-C₆ heteroalkyl, cycloalkyl, heterocyclyl, or —OR^{A}; or R^{B} and R^{C} together with the atom to which they are attached form a 3-7-membered heterocyclyl ring optionally substituted with one or more R⁸; each R^{D} is independently hydrogen, C₁-C₆ alkyl, C₂-C₆ alkenyl, C₂-C₆ alkynyl, C₁-C₆ heteroalkyl, C₁-C₆ haloalkyl, cycloalkyl, heterocyclyl, aryl, heteroaryl, C₁-C₆ alkylene-aryl, or C₁-C₆ alkylene-heteroaryl; each R⁷ is independently C₁-C₆ alkyl, C₁-C₆ heteroalkyl, C₁-C₆ haloalkyl, cycloalkyl, heterocyclyl, aryl, heteroaryl, halo, cyano, oxo, or —OR^{A}; each R⁸ is C₁-C₆-alkyl, halo, cyano, oxo, or —OR^{A1}; each R^{A1} is hydrogen or C₁-C₆-alkyl; m is 0, 1, 2, 3, or 4; and x is 0, 1, or 2.

In some embodiments, the compound of Formula (I) is a compound listed in Table 1, or a pharmaceutically acceptable salt, solvate, hydrate, tautomer, or stereoisomer thereof.

TABLE 1
Exemplary compounds of Formula (I)

| Compound No. | Structure |
|---|---|
| 100 | 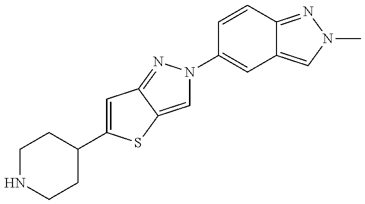 [SMILES: Cn1cc2cc(-n3cc4sc(C5CCNCC5)cc4n3)ccc2n1] |
| 101 | 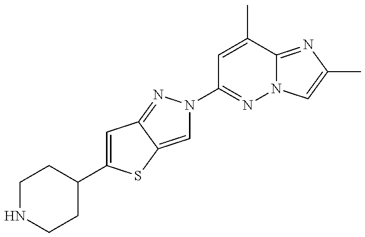 [SMILES: Cc1cn2nc(-n3cc4sc(C5CCNCC5)cc4n3)cc(C)c2n1] |
| 104 | 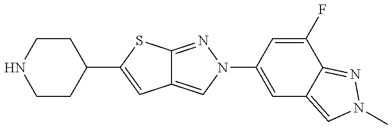 [SMILES: Cn1cc2cc(-n3cc4cc(C5CCNCC5)sc4n3)cc(F)c2n1] |
| 105 | 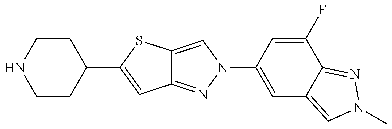 [SMILES: Cn1cc2cc(-n3cc4sc(C5CCNCC5)cc4n3)cc(F)c2n1] |
| 110 | 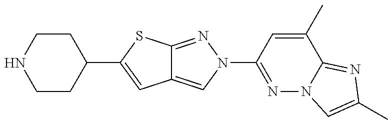 [SMILES: Cc1cn2nc(-n3cc4cc(C5CCNCC5)sc4n3)cc(C)c2n1] |
| 116 | 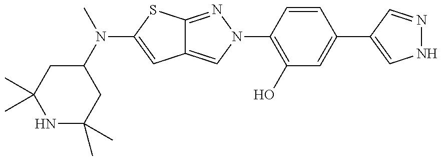 [SMILES: CN(c1cc2cn(-c3ccc(-c4cn[nH]c4)cc3O)nc2s1)C1CC(C)(C)NC(C)(C)C1] |
| 117 | 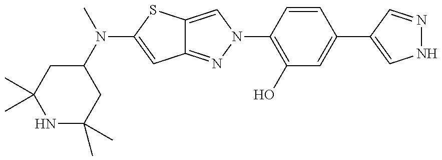 [SMILES: CN(c1cc2nn(-c3ccc(-c4cn[nH]c4)cc3O)cc2s1)C1CC(C)(C)NC(C)(C)C1] |
| 121 | 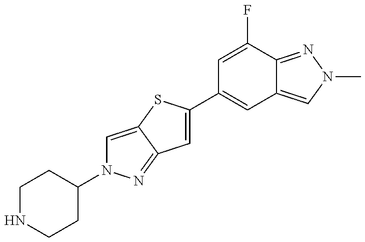 [SMILES: Cn1cc2cc(-c3cc4nn(C5CCNCC5)cc4s3)cc(F)c2n1] |
| 122 | 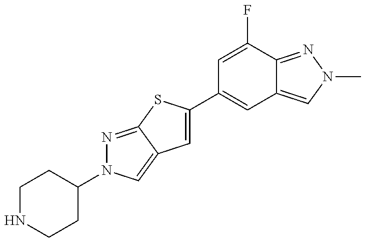 [SMILES: Cn1cc2cc(-c3cc4cn(C5CCNCC5)nc4s3)cc(F)c2n1] |
| 148 | 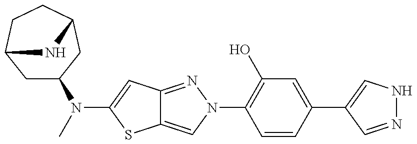 [SMILES: CN(c1cc2nn(-c3ccc(-c4cn[nH]c4)cc3O)cc2s1)[C@@H]1C[C@H]2CC[C@@H](C1)N2] |
| 149 | 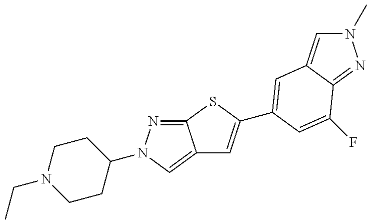 [SMILES: CCN1CCC(n2cc3cc(-c4cc(F)c5nn(C)cc5c4)sc3n2)CC1] |
| 150 | 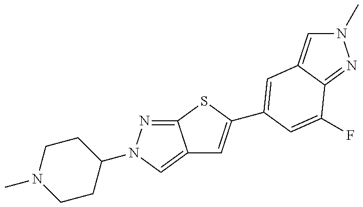 |
| 155 | 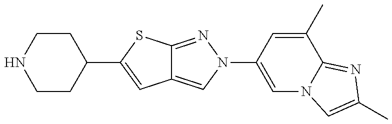 [SMILES: Cc1cn2cc(-n3cc4cc(C5CCNCC5)sc4n3)cc(C)c2n1] |
| 156 | 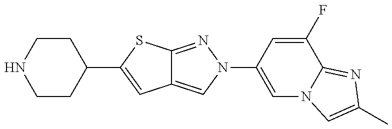 [SMILES: Cc1cn2cc(-n3cc4cc(C5CCNCC5)sc4n3)cc(F)c2n1] |
| 157 | 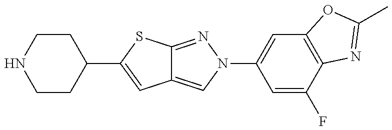 [SMILES: Cc1nc2c(F)cc(-n3cc4cc(C5CCNCC5)sc4n3)cc2o1] |
| 158 | 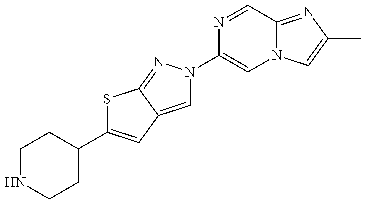 [SMILES: Cc1cn2cc(-n3cc4cc(C5CCNCC5)sc4n3)ncc2n1] |
| 159 | 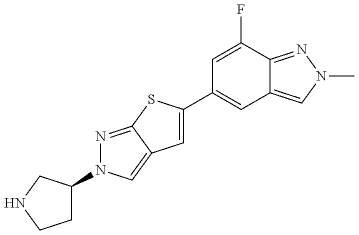 [SMILES: Cn1cc2cc(-c3cc4cn([C@H]5CCNC5)nc4s3)cc(F)c2n1] |
| 160 | 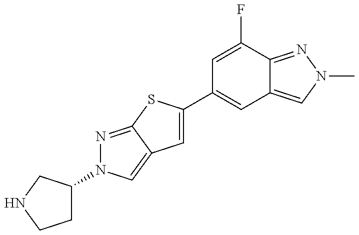 [SMILES: Cn1cc2cc(-c3cc4cn([C@@H]5CCNC5)nc4s3)cc(F)c2n1] |
| 161 | 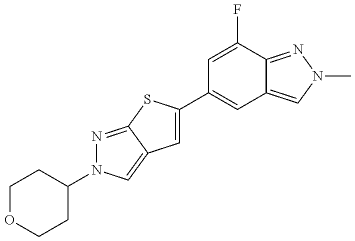 [SMILES: Cn1cc2cc(-c3cc4cn(C5CCOCC5)nc4s3)cc(F)c2n1] |
| 162 | 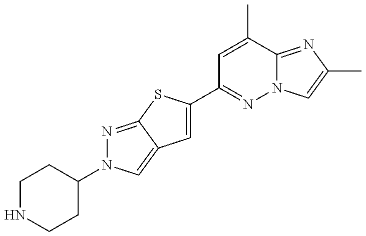 [SMILES: Cc1cn2nc(-c3cc4cn(C5CCNCC5)nc4s3)cc(C)c2n1] |
| 163 | 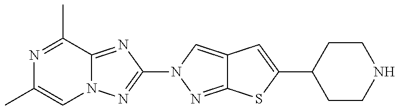 [SMILES: Cc1cn2nc(-n3cc4cc(C5CCNCC5)sc4n3)nc2c(C)n1] |
| 164 | 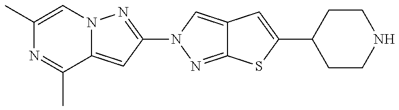 [SMILES: Cc1cn2nc(-n3cc4cc(C5CCNCC5)sc4n3)cc2c(C)n1] |
| 165 | 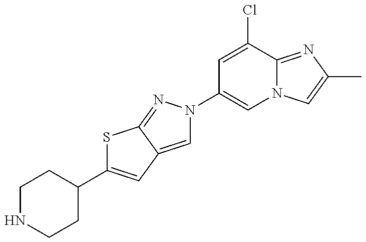 [SMILES: Cc1cn2cc(-n3cc4cc(C5CCNCC5)sc4n3)cc(Cl)c2n1] |
| 166 | 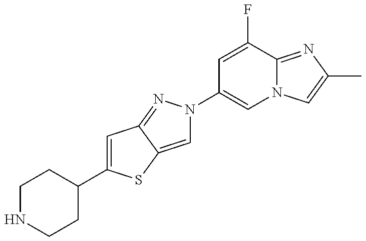 [SMILES: Cc1cn2cc(-n3cc4sc(C5CCNCC5)cc4n3)cc(F)c2n1] |
| 167 | 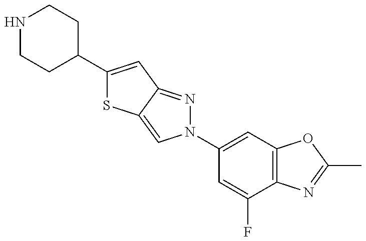 [SMILES: Cc1nc2c(F)cc(-n3cc4sc(C5CCNCC5)cc4n3)cc2o1] |
| 168 | 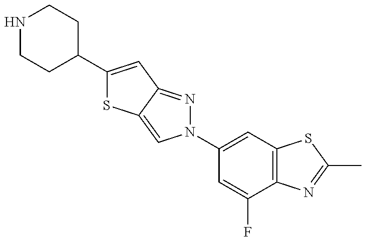 [SMILES: Cc1nc2c(F)cc(-n3cc4sc(C5CCNCC5)cc4n3)cc2s1] |
| 169 | 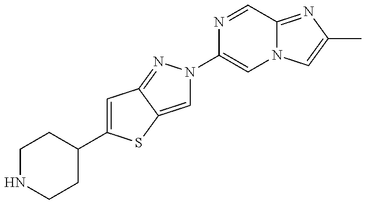 [SMILES: Cc1cn2cc(-n3cc4sc(C5CCNCC5)cc4n3)ncc2n1] |
| 170 | 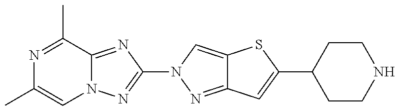 [SMILES: Cc1cn2nc(-n3cc4sc(C5CCNCC5)cc4n3)nc2c(C)n1] |
| 171 | 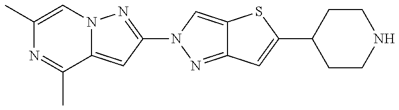 [SMILES: Cc1cn2nc(-n3cc4sc(C5CCNCC5)cc4n3)cc2c(C)n1] |
| 172 | 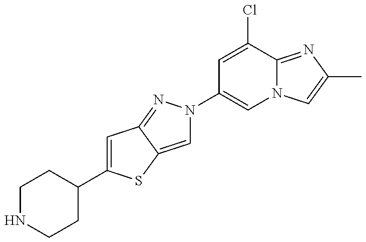 [SMILES: Cc1cn2cc(-n3cc4sc(C5CCNCC5)cc4n3)cc(Cl)c2n1] |
| 173 | 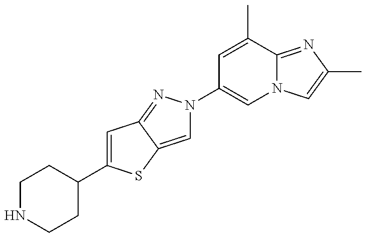 [SMILES: Cc1cn2cc(-n3cc4sc(C5CCNCC5)cc4n3)cc(C)c2n1] |
| 174 | 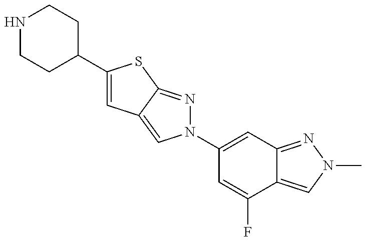 [SMILES: Cn1cc2c(F)cc(-n3cc4cc(C5CCNCC5)sc4n3)cc2n1] |
| 175 | 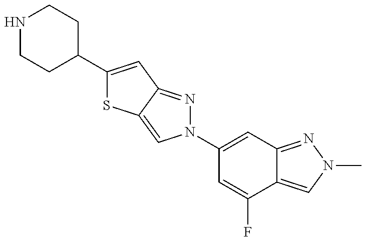 [SMILES: Cn1cc2c(F)cc(-n3cc4sc(C5CCNCC5)cc4n3)cc2n1] |
| 176 | 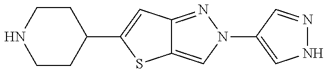 [SMILES: c1n[nH]cc1-n1cc2sc(C3CCNCC3)cc2n1] |
| 177 | 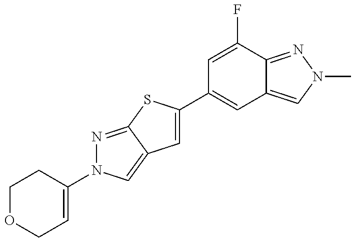 [SMILES: Cn1cc2cc(-c3cc4cn(C5=CCOCC5)nc4s3)cc(F)c2n1] |
| 178 | 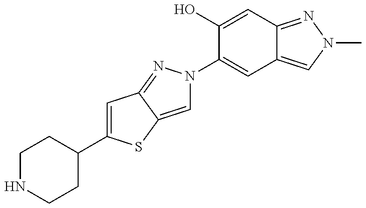 [SMILES: Cn1cc2cc(-n3cc4sc(C5CCNCC5)cc4n3)c(O)cc2n1] |
| 179 | 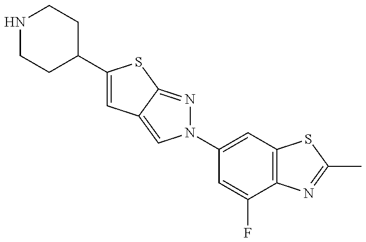 [SMILES: Cc1nc2c(F)cc(-n3cc4cc(C5CCNCC5)sc4n3)cc2s1] |
| 180 | 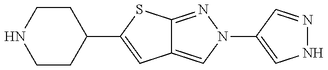 [SMILES: c1n[nH]cc1-n1cc2cc(C3CCNCC3)sc2n1] |
| 181 | 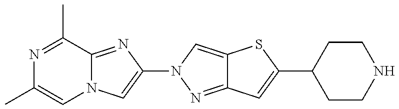 [SMILES: Cc1cn2cc(-n3cc4sc(C5CCNCC5)cc4n3)nc2c(C)n1] |
| 182 | 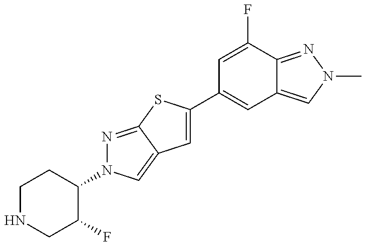 [SMILES: Cn1cc2cc(-c3cc4cn([C@H]5CCNC[C@H]5F)nc4s3)cc(F)c2n1] |
| 183 | 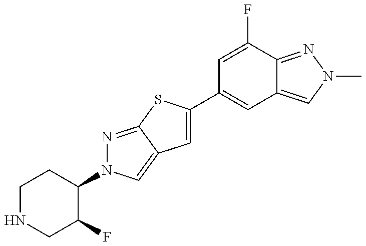 [SMILES: Cn1cc2cc(-c3cc4cn([C@@H]5CCNC[C@@H]5F)nc4s3)cc(F)c2n1] |
| 184 | 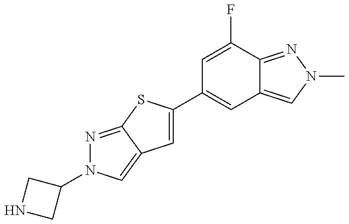 [SMILES: Cn1cc2cc(-c3cc4cn(C5CNC5)nc4s3)cc(F)c2n1] |
| 185 | 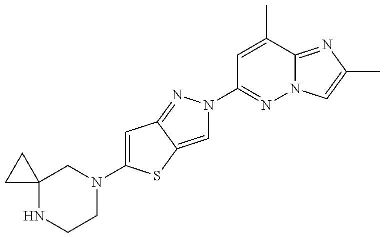 [SMILES: Cc1cn2nc(-n3cc4sc(N5CCNC6(CC6)C5)cc4n3)cc(C)c2n1] |
| 186 | 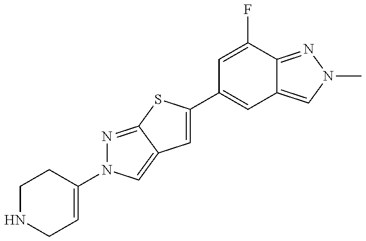 [SMILES: Cn1cc2cc(-c3cc4cn(C5=CCNCC5)nc4s3)cc(F)c2n1] |
| 187 | 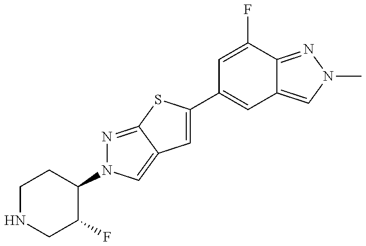 [SMILES: Cn1cc2cc(-c3cc4cn([C@@H]5CCNC[C@H]5F)nc4s3)cc(F)c2n1] |
| 188 | 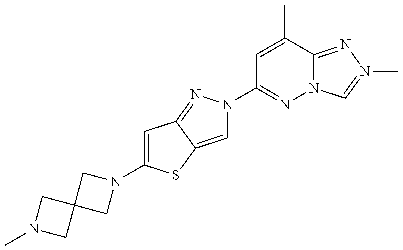 |
| 189 | 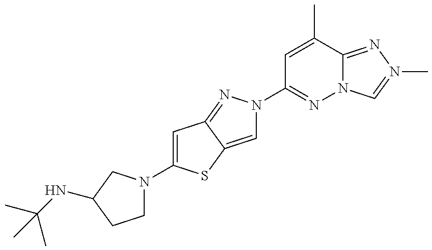 |
| 190 | 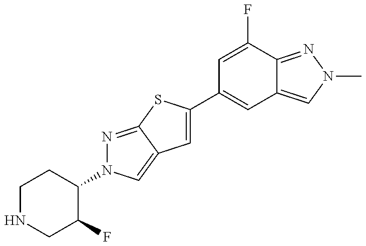 [SMILES: Cn1cc2cc(-c3cc4cn([C@H]5CCNC[C@@H]5F)nc4s3)cc(F)c2n1] |
| 207 | 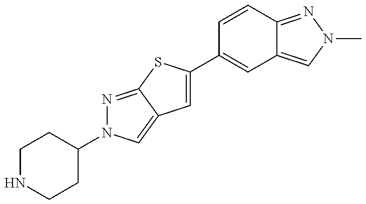 [SMILES: Cn1cc2cc(-c3cc4cn(C5CCNCC5)nc4s3)ccc2n1] |
| 208 | 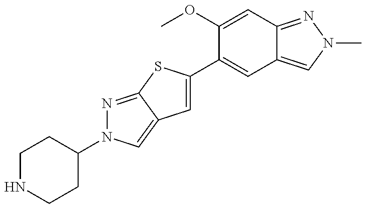 [SMILES: COc1cc2nn(C)cc2cc1-c1cc2cn(C3CCNCC3)nc2s1] |
| 209 | 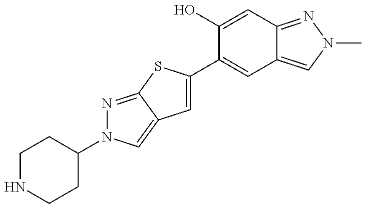 [SMILES: Cn1cc2cc(-c3cc4cn(C5CCNCC5)nc4s3)c(O)cc2n1] |
| 210 | 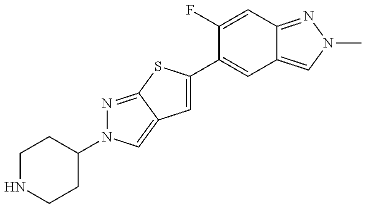 [SMILES: Cn1cc2cc(-c3cc4cn(C5CCNCC5)nc4s3)c(F)cc2n1] |
| 211 | 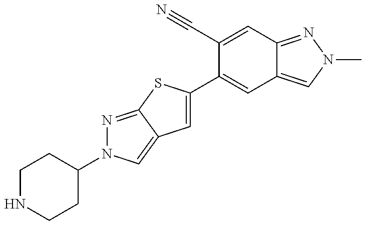 [SMILES: Cn1cc2cc(-c3cc4cn(C5CCNCC5)nc4s3)c(C#N)cc2n1] |
| 212 | 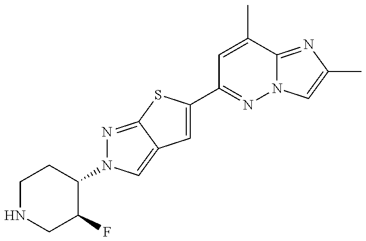 [SMILES: Cc1cn2nc(-c3cc4cn([C@H]5CCNC[C@@H]5F)nc4s3)cc(C)c2n1] |
| 213 | 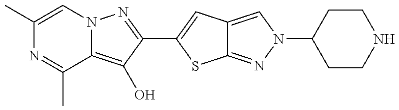 [SMILES: Cc1cn2nc(-c3cc4cn(C5CCNCC5)nc4s3)c(O)c2c(C)n1] |
| 214 | 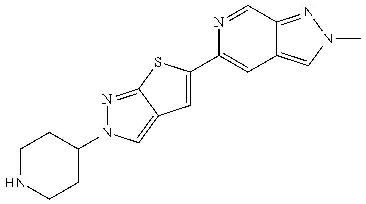 [SMILES: Cn1cc2cc(-c3cc4cn(C5CCNCC5)nc4s3)ncc2n1] |
| 215 | 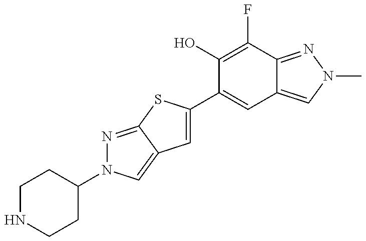 [SMILES: Cn1cc2cc(-c3cc4cn(C5CCNCC5)nc4s3)c(O)c(F)c2n1] |
| 216 | 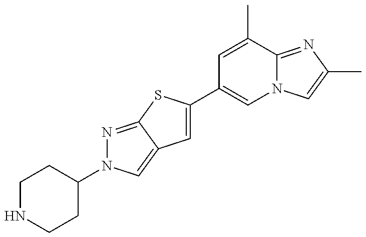 [SMILES: Cc1cn2cc(-c3cc4cn(C5CCNCC5)nc4s3)cc(C)c2n1] |
| 217 | 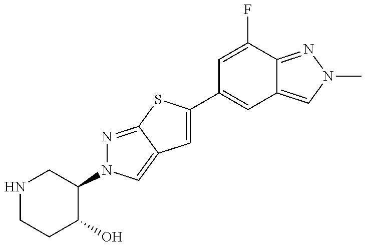 [SMILES: Cn1cc2cc(-c3cc4cn([C@@H]5CNCC[C@H]5O)nc4s3)cc(F)c2n1] |
| 218 | 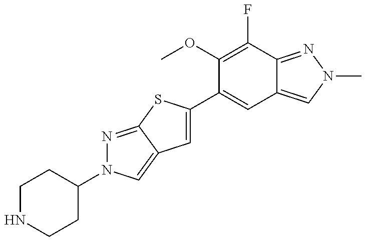 [SMILES: COc1c(-c2cc3cn(C4CCNCC4)nc3s2)cc2cn(C)nc2c1F] |
| 219 | 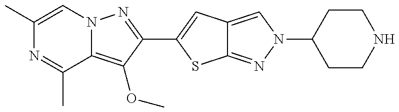 [SMILES: COc1c(-c2cc3cn(C4CCNCC4)nc3s2)nn2cc(C)nc(C)c12] |
| 220 | 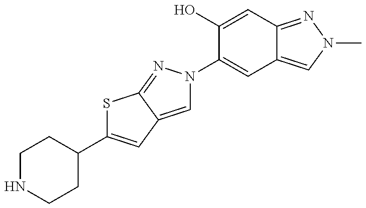 [SMILES: Cn1cc2cc(-n3cc4cc(C5CCNCC5)sc4n3)c(O)cc2n1] |
| 221 | 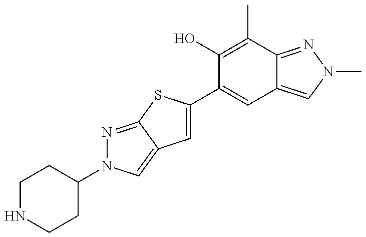 [SMILES: Cc1c(O)c(-c2cc3cn(C4CCNCC4)nc3s2)cc2cn(C)nc12] |
| 222 | 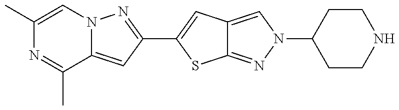 [SMILES: Cc1cn2nc(-c3cc4cn(C5CCNCC5)nc4s3)cc2c(C)n1] |
| 223 | 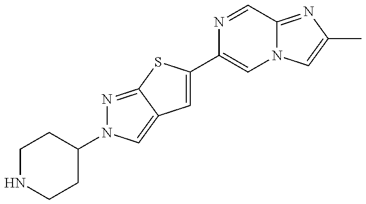 [SMILES: Cc1cn2cc(-c3cc4cn(C5CCNCC5)nc4s3)ncc2n1] |
| 224 | 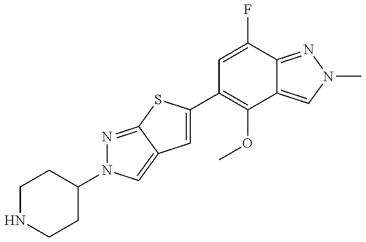 [SMILES: COc1c(-c2cc3cn(C4CCNCC4)nc3s2)cc(F)c2nn(C)cc12] |
| 225 | 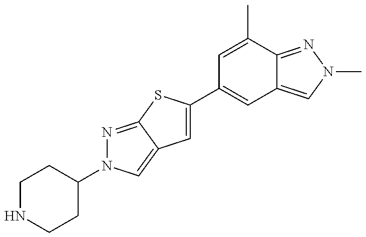 [SMILES: Cc1cc(-c2cc3cn(C4CCNCC4)nc3s2)cc2cn(C)nc12] |
| 226 | 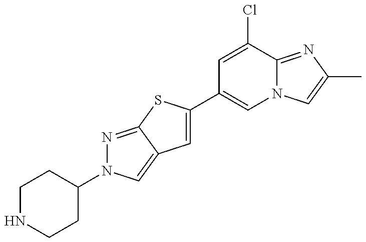 [SMILES: Cc1cn2cc(-c3cc4cn(C5CCNCC5)nc4s3)cc(Cl)c2n1] |
| 227 | 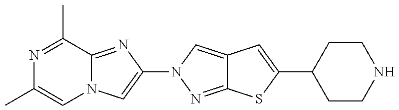 [SMILES: Cc1cn2cc(-n3cc4cc(C5CCNCC5)sc4n3)nc2c(C)n1] |
| 228 | 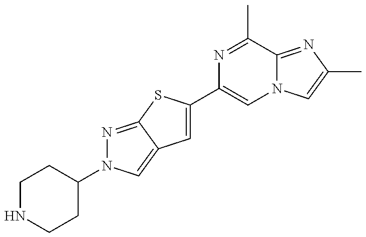 [SMILES: Cc1cn2cc(-c3cc4cn(C5CCNCC5)nc4s3)nc(C)c2n1] |
| 229 | 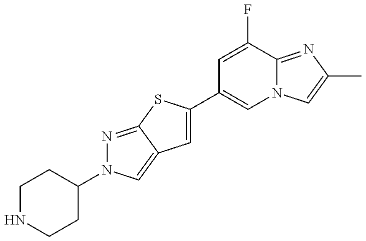 [SMILES: Cc1cn2cc(-c3cc4cn(C5CCNCC5)nc4s3)cc(F)c2n1] |
| 244 | 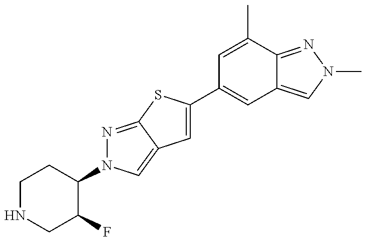 [SMILES: Cc1cc(-c2cc3cn([C@@H]4CCNC[C@@H]4F)nc3s2)cc2cn(C)nc12] |
| 245 | 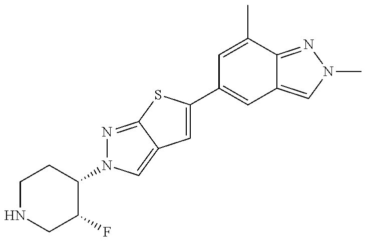 [SMILES: Cc1cc(-c2cc3cn([C@H]4CCNC[C@H]4F)nc3s2)cc2cn(C)nc12] |
| 246 | 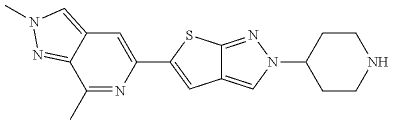 [SMILES: Cc1nc(-c2cc3cn(C4CCNCC4)nc3s2)cc2cn(C)nc12] |
| 248 | 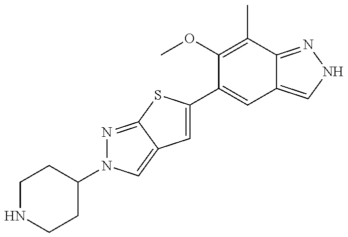 [SMILES: COc1c(-c2cc3cn(C4CCNCC4)nc3s2)cc2c[nH]nc2c1C] |
| 249 | 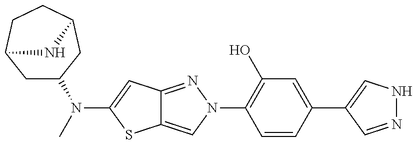 |
| 250 | 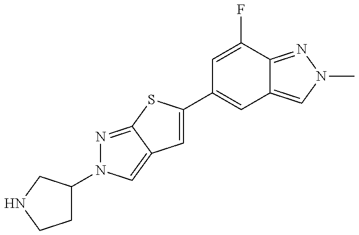 [SMILES: Cn1cc2cc(-c3cc4cn(C5CCNC5)nc4s3)cc(F)c2n1] |
| 251 | 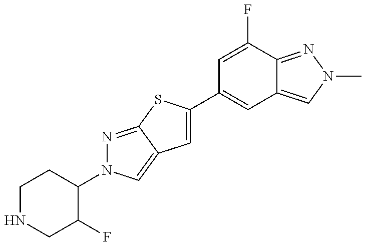 [SMILES: Cn1cc2cc(-c3cc4cn(C5CCNCC5F)nc4s3)cc(F)c2n1] |
| 252 | 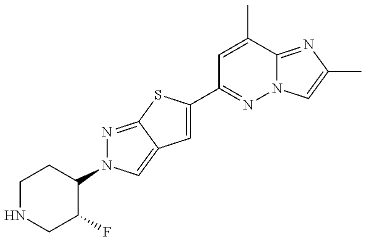 |
| 253 | 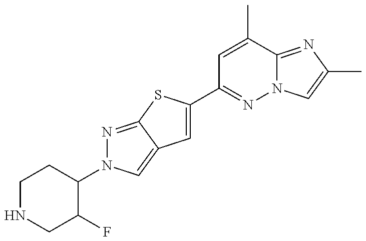 [SMILES: Cc1cn2nc(-c3cc4cn(C5CCNCC5F)nc4s3)cc(C)c2n1] |
| 254 | 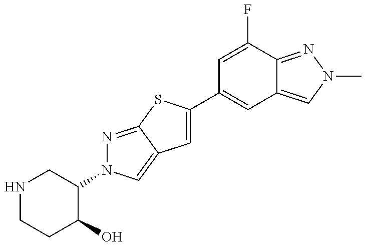 |
| 255 | 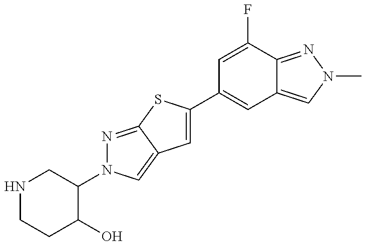 [SMILES: Cn1cc2cc(-c3cc4cn(C5CNCCC5O)nc4s3)cc(F)c2n1] |
| 256 | 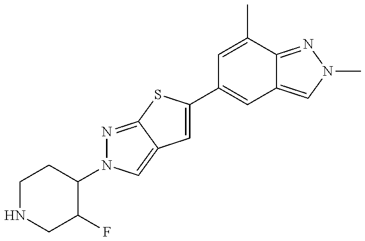 [SMILES: Cc1cc(-c2cc3cn(C4CCNCC4F)nc3s2)cc2cn(C)nc12] |

In some embodiments, for Formula (I), A is monocyclic heterocyclyl (e.g., piperidinyl); B is bicyclic heteroaryl (e.g., 2-methyl-2H-indazolyl); L¹ and L² are each absent; W is C(R⁵) (e.g., CH); X and Y are N; and R² is hydrogen. In some embodiments, the compound of Formula (I), (I-a), (I-b), and (I-e) is Compound 100, or a pharmaceutically acceptable salt, solvate, hydrate, tautomer, or stereoisomer thereof.

In some embodiments, for Formula (I), A is monocyclic heterocyclyl (e.g., piperidinyl); B is bicyclic heteroaryl (e.g., 2,8-dimethylimidazo[1,2-b]pyridazinyl); L¹ and L² are each absent; W is C(R⁵) (e.g., CH); X and Y are N; and R² is hydrogen. In some embodiments, the compound of Formula (I), (I-a), (I-b), and (I-e) is Compound 101, or a pharmaceutically acceptable salt, solvate, hydrate, tautomer, or stereoisomer thereof.

In some embodiments, for Formula (I), A is monocyclic heterocyclyl (e.g., piperidinyl); B is bicyclic heteroaryl (e.g., 7-fluoro-2-methyl-2H-indazolyl); L¹ and L² are each absent; Y is C(R⁵) (e.g., CH); X and W are N; and R² is hydrogen. In some embodiments, the compound of Formula (I), (I-a), (I-b), and (I-f) is Compound 104, or a pharmaceutically acceptable salt, solvate, hydrate, tautomer, or stereoisomer thereof.

In some embodiments, for Formula (I), A is monocyclic heterocyclyl (e.g., piperidinyl); B is bicyclic heteroaryl (e.g., 7-fluoro-2-methyl-2H-indazolyl); L¹ and L² are each absent; W is C(R⁵) (e.g., CH); X and Y are N; and R² is hydrogen. In some embodiments, the compound of Formula (I), (I-a), (I-b), and (I-e) is Compound 105, or a pharmaceutically acceptable salt, solvate, hydrate, tautomer, or stereoisomer thereof.

In some embodiments, for Formula (I), A is monocyclic heterocyclyl (e.g., piperidinyl); B is bicyclic heteroaryl (e.g., 2,8-dimethylimidazo[1,2-b]pyridazinyl); L¹ and L² are each absent; Y is C(R⁵) (e.g., CH); X and W are N; and R² is hydrogen. In some embodiments, the compound of Formula (I), (I-a), (I-b), and (I-f) is Compound 110, or a pharmaceutically acceptable salt, solvate, hydrate, tautomer, or stereoisomer thereof.

In some embodiments, for Formula (I), A is monocyclic heterocyclyl (e.g., 2,2,6,6-tetramethylpiperidinyl); B is monocyclic heteroaryl (e.g., pyrazolyl); L¹ is —N(R³)— (e.g., —N(CH₃)—); L² is C₆-C₁₂ arylene (e.g., phenyl) substituted with one R⁴; Y is C(R⁵) (e.g., CH); X and W are N; R² is hydrogen; and R⁴ is —OR^{A} (e.g., —OH). In some embodiments, the compound of Formula (I), (I-c), and (I-d) is Compound 116, or a pharmaceutically acceptable salt, solvate, hydrate, tautomer, or stereoisomer thereof.

In some embodiments, for Formula (I), A is monocyclic heterocyclyl (e.g., 2,2,6,6-tetramethylpiperidinyl); B is monocyclic heteroaryl (e.g., pyrazolyl); L¹ is —N(R³)— (e.g., —N(CH₃)—); L² is C₆-C₁₂ arylene (e.g., phenyl) substituted with one R⁴; W is C(R⁵) (e.g., CH); X and Y are N; R² is hydrogen; and R⁴ is —OR^{A} (e.g., —OH). In some embodiments, the compound of Formula (I), (I-c), and (I-d) is Compound 117, or a pharmaceutically acceptable salt, solvate, hydrate, tautomer, or stereoisomer thereof.

In some embodiments, for Formula (I), A is bicyclic heteroaryl (e.g., 7-fluoro-2-methyl-2H-indazolyl); B is monocyclic heterocyclyl (e.g., piperidinyl); L¹ and L² are each absent; W is C(R⁵) (e.g., CH); X and Y are N; and R² is hydrogen. In some embodiments, the compound of Formula (I), (I-a), (I-b), and (I-e) is Compound 121, or a pharmaceutically acceptable salt, solvate, hydrate, tautomer, or stereoisomer thereof.

In some embodiments, for Formula (I), A is bicyclic heteroaryl (e.g., 7-fluoro-2-methyl-2H-indazolyl); B is monocyclic heterocyclyl (e.g., piperidinyl); L¹ and L² are each absent; Y is C(R⁵) (e.g., CH); X and W are N; and R² is hydrogen. In some embodiments, the compound of Formula (I), (I-a), (I-b), and (I-f) is Compound 122, or a pharmaceutically acceptable salt, solvate, hydrate, tautomer, or stereoisomer thereof.

In some embodiments, for Formula (I), A is bicyclic heterocyclyl (e.g., 8-azabicyclo[3.2.1]octanyl); B is monocyclic heteroaryl (e.g., pyrazolyl); L¹ is —N(R³)— (e.g., —N(CH₃)—); L² is C₆-C₁₂ arylene (e.g., phenyl) substituted with one R⁴; W is C(R⁵) (e.g., CH); X and Y are N; R² is hydrogen; and R⁴ is —OR^{A} (e.g., —OH). In some embodiments, the compound of Formula (I), (I-c), and (I-d) is Compound 148, or a pharmaceutically acceptable salt, solvate, hydrate, tautomer, or stereoisomer thereof.

In some embodiments, for Formula (I), A is bicyclic heteroaryl (e.g., 7-fluoro-2-methyl-2H-indazolyl); B is monocyclic heterocyclyl (e.g., piperidinyl) substituted with one R¹; L¹ and L² are each absent; Y is C(R⁵) (e.g., CH); X and W are N; R¹ is C₁-C₆ alkyl (e.g., ethyl); and R² is hydrogen. In some embodiments, the compound of Formula (I), (I-a), (I-b), and (I-f) is Compound 149, or a pharmaceutically acceptable salt, solvate, hydrate, tautomer, or stereoisomer thereof.

In some embodiments, for Formula (I), A is bicyclic heteroaryl (e.g., 7-fluoro-2-methyl-2H-indazolyl); B is monocyclic heterocyclyl (e.g., piperidinyl) substituted with one R¹; L¹ and L² are each absent; Y is C(R⁵) (e.g., CH); X and W are N; R¹ is C₁-C₆ alkyl (e.g., methyl); and R² is hydrogen. In some embodiments, the compound of Formula (I), (I-a), (I-b), and (I-f) is Compound 150, or a pharmaceutically acceptable salt, solvate, hydrate, tautomer, or stereoisomer thereof.

In some embodiments, for Formula (I), A is monocyclic heterocyclyl (e.g., piperidinyl); B is bicyclic heteroaryl (e.g., 2,8-dimethylimidazo[1,2-a]pyridinyl); L¹ and L² are each absent; Y is C(R⁵) (e.g., CH); X and W are N; and R² is hydrogen. In some embodiments, the compound of Formula (I), (I-a), (I-b), and (I-f) is Compound 155, or a pharmaceutically acceptable salt, solvate, hydrate, tautomer, or stereoisomer thereof.

In some embodiments, for Formula (I), A is monocyclic heterocyclyl (e.g., piperidinyl); B is bicyclic heteroaryl (e.g., 8-fluoro-2-methylimidazo[1,2-a]pyridinyl); L¹ and L² are each absent; Y is C(R⁵) (e.g., CH); X and W are N; and R² is hydrogen. In some embodiments, the compound of Formula (I), (I-a), (I-b), and (I-f) is Compound 156, or a pharmaceutically acceptable salt, solvate, hydrate, tautomer, or stereoisomer thereof.

In some embodiments, for Formula (I), A is monocyclic heterocyclyl (e.g., piperidinyl); B is bicyclic heteroaryl (e.g 4-fluoro-2-methylbenzo[d]oxazolyl); L¹ and L² are each absent; Y is C(R⁵) (e.g., CH); X and W are N; and R² is hydrogen. In some embodiments, the compound of Formula (I), (I-a), (I-b), and (I-f) is Compound 157, or a pharmaceutically acceptable salt, solvate, hydrate, tautomer, or stereoisomer thereof.

In some embodiments, for Formula (I), A is monocyclic heterocyclyl (e.g., piperidinyl); B is bicyclic heteroaryl (e.g 2-methylimidazo[1,2-a]pyrazinyl); L¹ and L² are each absent; Y is C(R⁵) (e.g., CH); X and W are N; and R² is hydrogen. In some embodiments, the compound of Formula (I), (I-a), (I-b), and (I-f) is Compound 158, or a pharmaceutically acceptable salt, solvate, hydrate, tautomer, or stereoisomer thereof.

In some embodiments, for Formula (I), A is bicyclic heteroaryl (e.g 7-fluoro-2-methyl-2H-indazolyl); B is monocyclic heterocyclyl (e.g., pyrrolidinyl); L¹ and L² are each absent; Y is C(R⁵) (e.g., CH); X and W are N; and R² is hydrogen. In some embodiments, the compound of Formula (I), (I-a), (I-b), and (I-f) is Compound 159, 160, or a pharmaceutically acceptable salt, solvate, hydrate, tautomer, or stereoisomer thereof.

In some embodiments, for Formula (I), A is bicyclic heteroaryl (e.g 7-fluoro-2-methyl-2H-indazolyl); B is monocyclic heterocyclyl (e.g., tetrahydropyranyl); L¹ and L² are each absent; Y is C(R⁵) (e.g., CH); X and W are N; and R² is hydrogen. In some embodiments, the compound of Formula (I), (I-a), (I-b), and (I-f) is Compound 161, or a pharmaceutically acceptable salt, solvate, hydrate, tautomer, or stereoisomer thereof.

In some embodiments, for Formula (I), A is bicyclic heteroaryl (e.g 2,8-dimethylimidazo[1,2-b]pyridazinyl); B is monocyclic heterocyclyl (e.g., piperidinyl); L¹ and L² are each absent; Y is C(R⁵) (e.g., CH); X and W are N; and R² is hydrogen. In some embodiments, the compound of Formula (I), (I-a), (I-b), and (I-f) is Compound 162, or a pharmaceutically acceptable salt, solvate, hydrate, tautomer, or stereoisomer thereof.

In some embodiments, for Formula (I), A is monocyclic heterocyclyl (e.g., piperidinyl); B is bicyclic heteroaryl (e.g., 6,8-dimethyl-[1,2,4]triazolo[1,5-a]pyrazyl); L¹ and L² are each absent; Y is C(R⁵) (e.g., CH); X and W are N; and R² is hydrogen. In some embodiments, the compound of Formula (I), (I-a), (I-b), and (I-f) is Compound 163, or a pharmaceutically acceptable salt, solvate, hydrate, tautomer, or stereoisomer thereof.

In some embodiments, for Formula (I), A is monocyclic heterocyclyl (e.g., piperidinyl); B is bicyclic heteroaryl (e.g., 4,6-dimethylpyrazolo[1,5-a]pyrazyl); L¹ and L² are each absent; Y is C(R⁵) (e.g., CH); X and W are N; and R² is hydrogen. In some embodiments, the compound of Formula (I), (I-a), (I-b), and (I-f) is Compound 164, or a pharmaceutically acceptable salt, solvate, hydrate, tautomer, or stereoisomer thereof.

In some embodiments, for Formula (I), A is monocyclic heterocyclyl (e.g., piperidinyl); B is bicyclic heteroaryl (e.g., 8-chloro-2-methylimidazo[1,2-a]pyridinyl); L¹ and L² are each absent; Y is C(R⁵) (e.g., CH); X and W are N; and R² is hydrogen. In some embodiments, the compound of Formula (I), (I-a), (I-b), and (I-f) is Compound 165, or a pharmaceutically acceptable salt, solvate, hydrate, tautomer, or stereoisomer thereof.

In some embodiments, for Formula (I), A is monocyclic heterocyclyl (e.g., piperidinyl); B is bicyclic heteroaryl (e.g., 8-fluoro-2-methylimidazo[1,2-a]pyridinyl); L¹ and L² are each absent; W is C(R⁵) (e.g., CH); X and Y are N; and R² is hydrogen. In some embodiments, the compound of Formula (I), (I-a), (I-b), and (I-e) is Compound 166, or a pharmaceutically acceptable salt, solvate, hydrate, tautomer, or stereoisomer thereof.

In some embodiments, for Formula (I), A is monocyclic heterocyclyl (e.g., piperidinyl); B is bicyclic heteroaryl (e.g 4-fluoro-2-methylbenzo[d]oxazolyl); L¹ and L² are each absent; W is C(R⁵) (e.g., CH); X and Y are N; and R² is hydrogen. In some embodiments, the compound of Formula (I), (I-a), (I-b), and (I-e) is Compound 167, or a pharmaceutically acceptable salt, solvate, hydrate, tautomer, or stereoisomer thereof.

In some embodiments, for Formula (I), A is monocyclic heterocyclyl (e.g., piperidinyl); B is bicyclic heteroaryl (e.g 4-fluoro-2-methylbenzo[d]thiazolyl); L¹ and L² are each absent; W is C(R⁵) (e.g., CH); X and Y are N; and R² is hydrogen. In some embodiments, the compound of Formula (I), (I-a), (I-b), and (I-e) is Compound 168, or a pharmaceutically acceptable salt, solvate, hydrate, tautomer, or stereoisomer thereof.

In some embodiments, for Formula (I), A is monocyclic heterocyclyl (e.g., piperidinyl); B is bicyclic heteroaryl (e.g 2-methylimidazo[1,2-a]pyrazyl); L¹ and L² are each absent; W is C(R⁵) (e.g., CH); X and Y are N; and R² is hydrogen. In some embodiments, the compound of Formula (I), (I-a), (I-b), and (I-e) is Compound 169, or a pharmaceutically acceptable salt, solvate, hydrate, tautomer, or stereoisomer thereof.

In some embodiments, for Formula (I), A is monocyclic heterocyclyl (e.g., piperidinyl); B is bicyclic heteroaryl (e.g., 6,8-dimethyl-[1,2,4]triazolo[1,5-a]pyrazyl); L¹ and L² are each absent; W is C(R⁵) (e.g., CH); X and Y are N; and R² is hydrogen. In some embodiments, the compound of Formula (I), (I-a), (I-b), and (I-e) is Compound 170, or a pharmaceutically acceptable salt, solvate, hydrate, tautomer, or stereoisomer thereof.

In some embodiments, for Formula (I), A is monocyclic heterocyclyl (e.g., piperidinyl); B is bicyclic heteroaryl (e.g., 4,6-dimethylpyrazolo[1,5-a]pyrazyl); L¹ and L² are each absent; W is C(R⁵) (e.g., CH); X and Y are N; and R² is hydrogen. In some embodiments, the compound of Formula (I), (I-a), (I-b), and (I-e) is Compound 171, or a pharmaceutically acceptable salt, solvate, hydrate, tautomer, or stereoisomer thereof.

In some embodiments, for Formula (I), A is monocyclic heterocyclyl (e.g., piperidinyl); B is bicyclic heteroaryl (e.g., 8-chloro-2-methylimidazo[1,2-a]pyridinyl); L¹ and L² are each absent; W is C(R⁵) (e.g., CH); X and Y are N; and R² is hydrogen. In some embodiments, the compound of Formula (I), (I-a), (I-b), and (I-e) is Compound 172, or a pharmaceutically acceptable salt, solvate, hydrate, tautomer, or stereoisomer thereof.

In some embodiments, for Formula (I), A is monocyclic heterocyclyl (e.g., piperidinyl); B is bicyclic heteroaryl (e.g., 2,8-dimethylimidazo[1,2-a]pyridinyl); L¹ and L² are each absent; W is C(R⁵) (e.g., CH); X and Y are N; and R² is hydrogen. In some embodiments, the compound of Formula (I), (I-a), (I-b), and (I-e) is Compound 173, or a pharmaceutically acceptable salt, solvate, hydrate, tautomer, or stereoisomer thereof.

In some embodiments, for Formula (I), A is monocyclic heterocyclyl (e.g., piperidinyl); B is bicyclic heteroaryl (e.g., 4-fluoro-2-methyl-2H-indazolyl); L¹ and L² are each absent; Y is C(R⁵) (e.g., CH); X and W are N; and R² is hydrogen. In some embodiments, the compound of Formula (I), (I-a), (I-b), and (I-f) is Compound 174, or a pharmaceutically acceptable salt, solvate, hydrate, tautomer, or stereoisomer thereof.

In some embodiments, for Formula (I), A is monocyclic heterocyclyl (e.g., piperidinyl); B is bicyclic heteroaryl (e.g., 4-fluoro-2-methyl-2H-indazolyl); L¹ and L² are each absent; W is C(R⁵) (e.g., CH); X and Y are N; and R² is hydrogen. In some embodiments, the compound of Formula (I), (I-a), (I-b), and (I-e) is Compound 175, or a pharmaceutically acceptable salt, solvate, hydrate, tautomer, or stereoisomer thereof.

In some embodiments, for Formula (I), A is monocyclic heterocyclyl (e.g., piperidinyl); B is monocyclic heteroaryl (e.g., pyrazolyl); L¹ and L² are each absent; W is C(R⁵) (e.g., CH); X and Y are N; and R² is hydrogen. In some embodiments, the compound of Formula (I), (I-a), (I-b), and (I-e) is Compound 176, or a pharmaceutically acceptable salt, solvate, hydrate, tautomer, or stereoisomer thereof.

In some embodiments, for Formula (I), A is bicyclic heteroaryl (e.g 7-fluoro-2-methyl-2H-indazolyl); B is monocyclic heterocyclyl (e.g., 3,6-dihydro-2H-pyranyl); L¹ and L² are each absent; Y is C(R⁵) (e.g., CH); X and W are N; and R² is hydrogen. In some embodiments, the compound of Formula (I), (I-a), (I-b), and (I-f) is Compound 177, or a pharmaceutically acceptable salt, solvate, hydrate, tautomer, or stereoisomer thereof.

In some embodiments, for Formula (I), A is monocyclic heterocyclyl (e.g., piperidinyl); B is bicyclic heteroaryl (e.g., 6-hydroxy-2-methyl-2H-indazolyl); L¹ and L² are each absent; W is C(R⁵) (e.g., CH); X and Y are N; and R² is hydrogen. In some embodiments, the compound of Formula (I), (I-a), (I-b), and (I-e) is Compound 178, or a pharmaceutically acceptable salt, solvate, hydrate, tautomer, or stereoisomer thereof.

In some embodiments, for Formula (I), A is monocyclic heterocyclyl (e.g., piperidinyl); B is bicyclic heteroaryl (e.g 4-fluoro-2-methylbenzo[d]thiazolyl); L¹ and L² are each absent; Y is C(R⁵) (e.g., CH); X and W are N; and R² is hydrogen. In some embodiments, the compound of Formula (I), (I-a), (I-b), and (I-f) is Compound 179, or a pharmaceutically acceptable salt, solvate, hydrate, tautomer, or stereoisomer thereof.

In some embodiments, for Formula (I), A is monocyclic heterocyclyl (e.g., piperidinyl); B is monocyclic heteroaryl (e.g., pyrazolyl); L¹ and L² are each absent; Y is C(R⁵) (e.g., CH); X and W are N; and R² is hydrogen. In some embodiments, the compound of Formula (I), (I-a), (I-b), and (I-f) is Compound 180, or a pharmaceutically acceptable salt, solvate, hydrate, tautomer, or stereoisomer thereof.

In some embodiments, for Formula (I), A is monocyclic heterocyclyl (e.g., piperidinyl); B is monocyclic heteroaryl (e.g., 6,8-dimethylimidazo[1,2-a]pyrazyl); L¹ and L² are each absent; W is C(R⁵) (e.g., CH); X and Y are N; and R² is hydrogen. In some embodiments, the compound of Formula (I), (I-a), (I-b), and (I-e) is Compound 181, or a pharmaceutically acceptable salt, solvate, hydrate, tautomer, or stereoisomer thereof.

In some embodiments, for Formula (I), A is bicyclic heteroaryl (e.g 7-fluoro-2-methyl-2H-indazolyl); B is monocyclic heterocyclyl (e.g., 3-fluoropiperidinyl); L¹ and L² are each absent; Y is C(R⁵) (e.g., CH); X and W are N; and R² is hydrogen. In some embodiments, the compound of Formula (I), (I-a), (I-b), and (I-f) is Compound 182, 183, 187, 190, or a pharmaceutically acceptable salt, solvate, hydrate, tautomer, or stereoisomer thereof.

In some embodiments, for Formula (I), A is bicyclic heteroaryl (e.g 7-fluoro-2-methyl-2H-indazolyl); B is monocyclic heterocyclyl (e.g., azetidinyl); L¹ and L² are each absent; Y is C(R⁵) (e.g., CH); X and W are N; and R² is hydrogen. In some embodiments, the compound of Formula (I), (I-a), (I-b), and (I-f) is Compound 184 or a pharmaceutically acceptable salt, solvate, hydrate, tautomer, or stereoisomer thereof.

In some embodiments, for Formula (I), A is bicyclic heterocyclyl (e.g., 4,7-diazaspiro[2.5]octanyl); B is bicyclic heteroaryl (e.g., 2,8-dimethylimidazo[1,2-b]pyridazyl); L¹ and L² are each absent; W is C(R⁵) (e.g., CH); X and Y are N; and R² is hydrogen. In some embodiments, the compound of Formula (I), (I-a), (I-b), and (I-e) is Compound 185, or a pharmaceutically acceptable salt, solvate, hydrate, tautomer, or stereoisomer thereof.

In some embodiments, for Formula (I), A is bicyclic heteroaryl (e.g 7-fluoro-2-methyl-2H-indazolyl); B is monocyclic heterocyclyl (e.g., 1,2,3,6-tetrahydropyridinyl); L¹ and L² are each absent; Y is C(R⁵) (e.g., CH); X and W are N; and R² is hydrogen. In some embodiments, the compound of Formula (I), (I-a), (I-b), and (I-f) is Compound 186 or a pharmaceutically acceptable salt, solvate, hydrate, tautomer, or stereoisomer thereof.

In some embodiments, for Formula (I), A is bicyclic heterocyclyl (e.g., 2-methyl-2,6-diazaspiro[3.3]heptanyl); B is bicyclic heteroaryl (e.g., 2,8-dimethylimidazo[1,2-b]pyridazyl); L¹ and L² are each absent; W is C(R⁵) (e.g., CH); X and Y are N; and R² is hydrogen. In some embodiments, the compound of Formula (I), (I-a), (I-b), and (I-e) is Compound 188 or a pharmaceutically acceptable salt, solvate, hydrate, tautomer, or stereoisomer thereof.

In some embodiments, for Formula (I), A is monocyclic heterocyclyl (e.g., pyrrolidinyl) substituted with one R¹; B is bicyclic heteroaryl (e.g., 2,8-dimethylimidazo[1,2-b]pyridazyl); L¹ and L² are each absent; W is C(R⁵) (e.g., CH); X and Y are N; R¹ is —NR^{B}R^{C} (e.g., —NH(^{t-}Bu)); and R² is hydrogen. In some embodiments, the compound of Formula (I), (I-a), (I-b), and (I-e) is Compound 189 or a pharmaceutically acceptable salt, solvate, hydrate, tautomer, or stereoisomer thereof.

In some embodiments, for Formula (I), A is bicyclic heteroaryl (e.g., 2H-indazolyl) substituted with one R¹; B is monocyclic heterocyclyl (e.g., piperidinyl); L¹ and L² are each absent; W and X are N; Y is C(R⁵) (e.g., CH); R¹ is C₁-C₆ alkyl (e.g., —CH₃); and R² is hydrogen. In some embodiments, the compound of Formula (I), (I-a), (I-b), and (I-f) is Compound 207 or a pharmaceutically acceptable salt, solvate, hydrate, tautomer, or stereoisomer thereof.

In some embodiments, for Formula (I), A is bicyclic heteroaryl (e.g., 2H-indazolyl) substituted with two R¹; B is monocyclic heterocyclyl (e.g., piperidinyl); L¹ and L² are each absent; W and X are N; Y is C(R⁵) (e.g., CH); one R¹ is C₁-C₆ alkyl (e.g., —CH₃) and the other R¹ is —OR^{A} (e.g., —OCH₃); and R² is hydrogen. In some embodiments, the compound of Formula (I), (I-a), (I-b), and (I-f) is Compound 208 or a pharmaceutically acceptable salt, solvate, hydrate, tautomer, or stereoisomer thereof.

In some embodiments, for Formula (I), A is bicyclic heteroaryl (e.g., 2H-indazolyl) substituted with two R¹; B is monocyclic heterocyclyl (e.g., piperidinyl); L¹ and L² are each absent; W and X are N; Y is C(R⁵) (e.g., CH); one R¹ is C₁-C₆ alkyl (e.g., —CH₃) and the other R¹ is —OR^{A} (e.g., —OH); and R² is hydrogen. In some embodiments, the compound of Formula (I), (I-a), (I-b), and (I-f) is Compound 209 or a pharmaceutically acceptable salt, solvate, hydrate, tautomer, or stereoisomer thereof.

In some embodiments, for Formula (I), A is bicyclic heteroaryl (e.g., 2H-indazolyl) substituted with two R¹; B is monocyclic heterocyclyl (e.g., piperidinyl); L¹ and L² are each absent; W and X are N; Y is C(R⁵) (e.g., CH); one R¹ is C₁-C₆ alkyl (e.g., —CH₃) and the other R¹ is halo (e.g., —F); and R² is hydrogen. In some embodiments, the compound of Formula (I), (I-a), (I-b), and (I-f) is Compound 210 or a pharmaceutically acceptable salt, solvate, hydrate, tautomer, or stereoisomer thereof.

In some embodiments, for Formula (I), A is bicyclic heteroaryl (e.g., 2H-indazolyl) substituted with two R¹; B is monocyclic heterocyclyl (e.g., piperidinyl); L¹ and L² are each absent; W and X are N; Y is C(R⁵) (e.g., CH); one R¹ is C₁-C₆ alkyl (e.g., —CH₃) and the other R¹ is cyano; and R² is hydrogen. In some embodiments, the compound of Formula (I), (I-a), (I-b), and (I-f) is Compound 211 or a pharmaceutically acceptable salt, solvate, hydrate, tautomer, or stereoisomer thereof.

In some embodiments, for Formula (I), A is bicyclic heteroaryl (e.g., imidazo[1,2-b]pyridazyl) substituted with two R¹; B is monocyclic heterocyclyl (e.g., piperidinyl) substituted with one R¹; L¹ and L² are each absent; W and X are N; Y is C(R⁵) (e.g., CH); each R¹ is independently selected from C₁-C₆ alkyl (e.g., —CH₃) and halo (e.g., —F); and R² is hydrogen. In some embodiments, the compound of Formula (I), (I-a), (I-b), and (I-f) is Compound 212 or a pharmaceutically acceptable salt, solvate, hydrate, tautomer, or stereoisomer thereof.

In some embodiments, for Formula (I), A is bicyclic heteroaryl (e.g., pyrazolo[1,5-a]pyrazyl) substituted with three R¹; B is monocyclic heterocyclyl (e.g., piperidinyl); L¹ and L² are each absent; W and X are N; Y is C(R⁵) (e.g., CH); each R¹ is independently selected from C₁-C₆ alkyl (e.g., —CH₃) and —OR^{A} (e.g., —OH); and R² is hydrogen. In some embodiments, the compound of Formula (I), (I-a), (I-b), and (I-f) is Compound 213 or a pharmaceutically acceptable salt, solvate, hydrate, tautomer, or stereoisomer thereof.

In some embodiments, for Formula (I), A is bicyclic heteroaryl (e.g., 2H-pyrazolo[3,4-c]pyridyl) substituted with one R¹; B is monocyclic heterocyclyl (e.g., piperidinyl); L¹ and L² are each absent; W and X are N; Y is C(R⁵) (e.g., CH); each R¹ is C₁-C₆ alkyl (e.g., —CH₃); and R² is hydrogen. In some embodiments, the compound of Formula (I), (I-a), (I-b), and (I-f) is Compound 214 or a pharmaceutically acceptable salt, solvate, hydrate, tautomer, or stereoisomer thereof.

In some embodiments, for Formula (I), A is bicyclic heteroaryl (e.g., 2H-indazolyl) substituted with three R¹; B is monocyclic heterocyclyl (e.g., piperidinyl); L¹ and L² are each absent; W and X are N; Y is C(R⁵) (e.g., CH); each R¹ is independently selected from C₁-C₆ alkyl (e.g., —CH₃), halo (e.g., —F), and —OR^{A} (e.g., —OH); and R² is hydrogen. In some embodiments, the compound of Formula (I), (I-a), (I-b), and (I-f) is Compound 215 or a pharmaceutically acceptable salt, solvate, hydrate, tautomer, or stereoisomer thereof.

In some embodiments, for Formula (I), A is bicyclic heteroaryl (e.g., imidazo[1,2-a]pyridyl) substituted with two R¹; B is monocyclic heterocyclyl (e.g., piperidinyl); L¹ and L² are each absent; W and X are N; Y is C(R⁵) (e.g., CH); each R¹ is C₁-C₆ alkyl (e.g., —CH₃); and R² is hydrogen. In some embodiments, the compound of Formula (I), (I-a), (I-b), and (I-f) is Compound 216 or a pharmaceutically acceptable salt, solvate, hydrate, tautomer, or stereoisomer thereof.

In some embodiments, for Formula (I), A is bicyclic heteroaryl (e.g., 2H-indazolyl) substituted with two R¹; B is monocyclic heterocyclyl (e.g., piperidinyl) substituted with one R¹; L¹ and L² are each absent; W and X are N; Y is C(R⁵) (e.g., CH); each R¹ is independently selected from C₁-C₆ alkyl (e.g., —CH₃), halo (e.g., —F), and —OR^{A} (e.g., —OH); and R² is hydrogen. In some embodiments, the compound of Formula (I), (I-a), (I-b), and (I-f) is Compound 217 or a pharmaceutically acceptable salt, solvate, hydrate, tautomer, or stereoisomer thereof.

In some embodiments, for Formula (I), A is bicyclic heteroaryl (e.g., 2H-indazolyl) substituted with three R¹; B is monocyclic heterocyclyl (e.g., piperidinyl); L¹ and L² are each absent; W and X are N; Y is C(R⁵) (e.g., CH); each R¹ is independently selected from C₁-C₆ alkyl (e.g., —CH₃), halo (e.g., —F), and —OR^{A} (e.g., —OCH₃); and R² is hydrogen. In some embodiments, the compound of Formula (I), (I-a), (I-b), and (I-f) is Compound 218, 224 or a pharmaceutically acceptable salt, solvate, hydrate, tautomer, or stereoisomer thereof.

In some embodiments, for Formula (I), A is bicyclic heteroaryl (e.g., pyrazolo[1,5-a]pyrazyl) substituted with three R¹; B is monocyclic heterocyclyl (e.g., piperidinyl); L¹ and L² are each absent; W and X are N; Y is C(R⁵) (e.g., CH); each R¹ is independently selected from C₁-C₆ alkyl (e.g., —CH₃) and —OR^{A} (e.g., —OCH₃); and R² is hydrogen. In some embodiments, the compound of Formula (I), (I-a), (I-b), and (I-f) is Compound 219 or a pharmaceutically acceptable salt, solvate, hydrate, tautomer, or stereoisomer thereof.

In some embodiments, for Formula (I), A is monocyclic heterocyclyl (e.g., piperidinyl); B is bicyclic heteroaryl (e.g., 2H-indazolyl) substituted with two R¹; L¹ and L² are each absent; W and X are N; Y is C(R⁵) (e.g., CH); one R¹ is C₁-C₆ alkyl (e.g., —CH₃) and the other R¹ is —OR^{A} (e.g., —OH); and R² is hydrogen. In some embodiments, the compound of Formula (I), (I-a), (I-b), and (I-f) is Compound 220 or a pharmaceutically acceptable salt, solvate, hydrate, tautomer, or stereoisomer thereof.

In some embodiments, for Formula (I), A is bicyclic heteroaryl (e.g., 2H-indazolyl) substituted with three R¹; B is monocyclic heterocyclyl (e.g., piperidinyl); L¹ and L² are each absent; W and X are N; Y is C(R⁵) (e.g., CH); each R¹ is independently selected from C₁-C₆ alkyl (e.g., —CH₃) and —OR^{A} (e.g., —OCH₃); and R² is hydrogen. In some embodiments, the compound of Formula (I), (I-a), (I-b), and (I-f) is Compound 221 or a pharmaceutically acceptable salt, solvate, hydrate, tautomer, or stereoisomer thereof.

In some embodiments, for Formula (I), A is bicyclic heteroaryl (e.g., pyrazolo[1,5-a]pyrazyl) substituted with two R¹; B is monocyclic heterocyclyl (e.g., piperidinyl); L¹ and L² are each absent; W and X are N; Y is C(R⁵) (e.g., CH); each R¹ is independently selected from C₁-C₆ alkyl (e.g., —CH₃); and R² is hydrogen. In some embodiments, the compound of Formula (I), (I-a), (I-b), and (I-f) is Compound 222 or a pharmaceutically acceptable salt, solvate, hydrate, tautomer, or stereoisomer thereof.

In some embodiments, for Formula (I), A is bicyclic heteroaryl (e.g., imidazo[1,2-a]pyrazinyl) substituted with one R¹; B is monocyclic heterocyclyl (e.g., piperidinyl); L¹ and L² are each absent; W and X are N; Y is C(R⁵) (e.g., CH); R¹ is C₁-C₆ alkyl (e.g., —CH₃); and R² is hydrogen. In some embodiments, the compound of Formula (I), (I-a), (I-b), and (I-f) is Compound 223 or a pharmaceutically acceptable salt, solvate, hydrate, tautomer, or stereoisomer thereof.

In some embodiments, for Formula (I), A is bicyclic heteroaryl (e.g., 2H-indazolyl) substituted with two R¹; B is monocyclic heterocyclyl (e.g., piperidinyl); L¹ and L² are each absent; W and X are N; Y is C(R⁵) (e.g., CH); each R¹ is independently selected from C₁-C₆ alkyl (e.g., —CH₃); and R² is hydrogen. In some embodiments, the compound of Formula (I), (I-a), (I-b), and (I-f) is Compound 225 or a pharmaceutically acceptable salt, solvate, hydrate, tautomer, or stereoisomer thereof.

In some embodiments, for Formula (I), A is bicyclic heteroaryl (e.g., imidazo[1,2-a]pyridyl) substituted with two R¹; B is monocyclic heterocyclyl (e.g., piperidinyl); L¹ and L² are each absent; W and X are N; Y is C(R⁵) (e.g., CH); each R¹ is independently selected from C₁-C₆ alkyl (e.g., —CH₃) and halo (e.g., —Cl); and R² is hydrogen. In some embodiments, the compound of Formula (I), (I-a), (I-b), and (I-f) is Compound 226 or a pharmaceutically acceptable salt, solvate, hydrate, tautomer, or stereoisomer thereof.

In some embodiments, for Formula (I), A is monocyclic heterocyclyl (e.g., piperidinyl); B is bicyclic heteroaryl (e.g., imidazo[1,2-a]pyrazyl) substituted with two R¹; L¹ and L² are each absent; W and X are N; Y is C(R⁵) (e.g., CH); each R¹ is independently C₁-C₆ alkyl (e.g., —CH₃); and R² is hydrogen. In some embodiments, the compound of Formula (I), (I-a), (I-b), and (I-f) is Compound 227 or a pharmaceutically acceptable salt, solvate, hydrate, tautomer, or stereoisomer thereof.

In some embodiments, for Formula (I), A is bicyclic heteroaryl (e.g., imidazo[1,2-a]pyrazyl) substituted with two R¹; B is monocyclic heterocyclyl (e.g., piperidinyl); L¹ and L² are each absent; W and X are N; Y is C(R⁵) (e.g., CH); each R¹ is independently C₁-C₆ alkyl (e.g., —CH₃); and R² is hydrogen. In some embodiments, the compound of Formula (I), (I-a), (I-b), and (I-f) is Compound 228 or a pharmaceutically acceptable salt, solvate, hydrate, tautomer, or stereoisomer thereof.

In some embodiments, for Formula (I), A is bicyclic heteroaryl (e.g., imidazo[1,2-a]pyridyl) substituted with two R¹; B is monocyclic heterocyclyl (e.g., piperidinyl); L¹ and L² are each absent; W and X are N; Y is C(R⁵) (e.g., CH); each R¹ is independently selected from C₁-C₆ alkyl (e.g., —CH₃) and halo (e.g., —F); and R² is hydrogen. In some embodiments, the compound of Formula (I), (I-a), (I-b), and (I-f) is Compound 229 or a pharmaceutically acceptable salt, solvate, hydrate, tautomer, or stereoisomer thereof.

In some embodiments, the compound of Formula (II) is a compound of Formula (II-a):

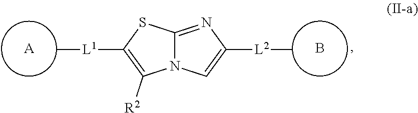

or a pharmaceutically acceptable salt, solvate, hydrate, tautomer, or stereoisomer thereof, wherein A and B are each independently cycloalkyl, heterocyclyl, aryl, or heteroaryl, each of which is optionally substituted with one or more R¹; L¹ is absent, C₁-C₆-alkylene, C₁-C₆-heteroalkylene, —O—, —C(O)—, —N(R³)—, —N(R³)C(O)—, or C(O)N(R³)—, wherein each alkylene and heteroalkylene is optionally substituted with one or more R⁴; L² is absent, C₁-C₆-alkylene, C₁-C₆-heteroalkylene, C₆-C₁₂-arylene, C₅-C₁₂-heteroarylene, —O—, —C(O)—, —N(R³)—, —N(R³)C(O)—, or —C(O)N(R³)—, wherein each alkylene, heteroalkylene, arylene, and heteroarylene is optionally substituted with one or more R⁴; each R¹ is independently hydrogen, C₁-C₆-alkyl, C₂-C₆-alkenyl, C₂-C₆-alkynyl, C₁-C₆-heteroalkyl, C₁-C₆-haloalkyl, cycloalkyl, heterocyclyl, aryl, C₁-C₆ alkylene-aryl, C₁-C₆ alkenylene-aryl, C₁-C₆ alkylene-heteroaryl, heteroaryl, halo, cyano, oxo, —OR^{A}, —NR^{B}R^{C}, —NR^{B}C(O)R^{D}, —NO₂, —C(O)NR^{B}R^{C}, —C(O)R^{D}, —C(O)OR^{D}, or —S(O)ₓR^{D}, wherein each alkyl, alkylene, alkenyl, alkynyl, heteroalkyl, haloalkyl, cycloalkyl, heterocyclyl, aryl, and heteroaryl is optionally substituted with one or more R⁶; or two R¹ groups, together with the atoms to which they are attached, form a 3-7-membered cycloalkyl, heterocyclyl, aryl, or heteroaryl, wherein each cycloalkyl, heterocyclyl, aryl, and heteroaryl is optionally substituted with one or more R⁶; R² is hydrogen, halo, cyano, C₁-C₆-alkyl, C₂-C₆-alkenyl, C₂-C₆-alkynyl, C₁-C₆-heteroalkyl, or C₁-C₆-haloalkyl; each R³ is independently hydrogen, C₁-C₆-alkyl, or C₁-C₆-haloalkyl; each R⁴ is independently C₁-C₆-alkyl, C₁-C₆-heteroalkyl, C₁-C₆-haloalkyl, cycloalkyl, halo, cyano, oxo, —OR^{A}, —NR^{B}R^{C}, —C(O)R^{D}, or —C(O)OR^{D}; each R⁶ is independently C₁-C₆-alkyl, C₂-C₆-alkenyl, C₂-C₆-alkynyl, C₁-C₆-heteroalkyl, C₁-C₆-haloalkyl, cycloalkyl, heterocyclyl, aryl, heteroaryl, halo, cyano, oxo, —OR^{A}, —NR^{B}R^{C}, NR^{B}C(O)R^{D}, —NO₂, —C(O)NR^{B}R^{C}, —C(O)R^{D}, —C(O)OR^{D}, or —S(O)ₓR^{D}, wherein each of alkyl, alkenyl, alkynyl, heteroalkyl, haloalkyl, cycloalkyl, heterocyclyl, aryl, and heteroaryl is optionally substituted with one or more R⁷; each R^{A} is independently hydrogen, C₁-C₆ alkyl, C₁-C₆ haloalkyl, aryl, heteroaryl, C₁-C₆ alkylene-aryl, C₁-C₆ alkylene-heteroaryl, —C(O)R^{D}, or —S(O)ₓR^{D}; each of R^{B} and R^{C} is independently hydrogen, C₁-C₆ alkyl, C₁-C₆ heteroalkyl, cycloalkyl, heterocyclyl, or —OR^{A}; or R^{B} and R^{C} together with the atom to which they are attached form a 3-7-membered heterocyclyl ring optionally substituted with one or more R⁸; each R^{D} is independently hydrogen, C₁-C₆ alkyl, C₂-C₆ alkenyl, C₂-C₆ alkynyl, C₁-C₆ heteroalkyl, C₁-C₆ haloalkyl, cycloalkyl, heterocyclyl, aryl, heteroaryl, C₁-C₆ alkylene-aryl, or C₁-C₆ alkylene-heteroaryl; each R⁷ is independently C₁-C₆ alkyl, C₁-C₆ heteroalkyl, C₁-C₆ haloalkyl, cycloalkyl, heterocyclyl, aryl, heteroaryl, halo, cyano, oxo, or —OR^{A}; each R⁸ is C₁-C₆-alkyl, halo, cyano, oxo, or —OR^{A1}; each R^{A1} is hydrogen or C₁-C₆-alkyl; and x is 0, 1, or 2.

In some embodiments, for Formula (II), one of A and B is independently a monocyclic heteroaryl or bicyclic heteroaryl, each of which is optionally substituted with one or more R¹. In some embodiments, one of A and B is independently a bicyclic heteroaryl optionally substituted with one or more R¹. In some embodiments, one of A and B is independently a nitrogen-containing heteroaryl optionally substituted with one or more R¹.

In some embodiments, one of A and B is independently selected from

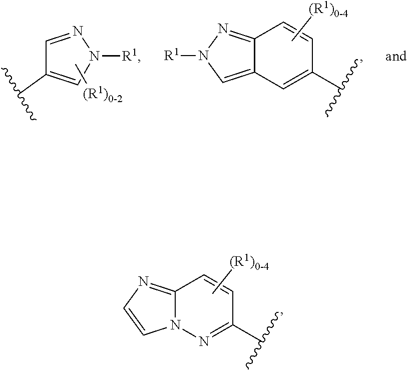

wherein R¹ is as described herein. In some embodiments, one of A and B is independently selected from,

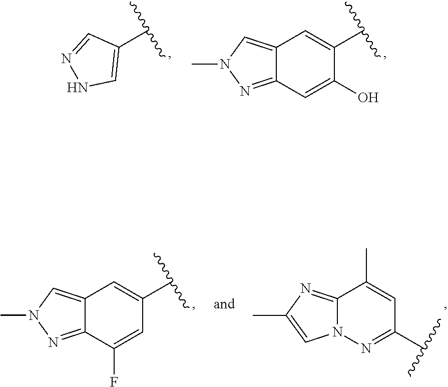

wherein R¹ is as described herein. In some embodiments, one of A and B is independently a monocyclic heterocyclyl or bicyclic heterocyclyl, each of which is optionally substituted with one or more R¹.

In some embodiments, one of A and B is independently a nitrogen-containing heterocyclyl optionally substituted with one or more R¹. In some embodiments, one of A and B is independently

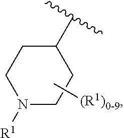

wherein R¹ is as described herein. In some embodiments, one of A and B is independently is selected from

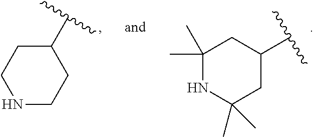 [SMILES: *C1CCNCC1 ; *C1CC(C)(C)NC(C)(C)C1]

In some embodiments, one of A and B is independently is

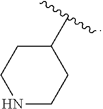 [SMILES: *C1CCNCC1]

In some embodiments, each of L¹ and L² is independently absent, —N(R³)— (e.g., —N(CH₃)—), or C₆-C₁₂-arylene, wherein arylene is optionally substituted with one or more R¹. In some embodiments, one of L¹ and L² is independently absent. In some embodiments, each of L¹ and L² is independently absent. In some embodiments, Y is N. In some embodiments, R² is hydrogen.

In some embodiments, the compound of Formula (II) is a compound of Formula (II-b):

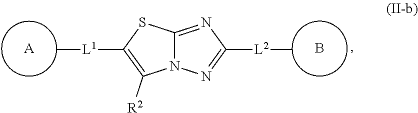

or a pharmaceutically acceptable salt, solvate, hydrate, tautomer, or stereoisomer thereof, wherein A and B are each independently cycloalkyl, heterocyclyl, aryl, or heteroaryl, each of which is optionally substituted with one or more R¹; L¹ is absent, C₁-C₆-alkylene, C₁-C₆-heteroalkylene, —O—, —C(O)—, —N(R³)—, —N(R³)C(O)—, or C(O)N(R³)—, wherein each alkylene and heteroalkylene is optionally substituted with one or more R⁴; L² is absent, C₁-C₆-alkylene, C₁-C₆-heteroalkylene, C₆-C₁₂-arylene, C₅-C₁₂-heteroarylene, —O—, —C(O)—, —N(R³)—, —N(R³)C(O)—, or —C(O)N(R³)—, wherein each alkylene, heteroalkylene, arylene, and heteroarylene is optionally substituted with one or more R⁴; each R¹ is independently hydrogen, C₁-C₆-alkyl, C₂-C₆-alkenyl, C₂-C₆-alkynyl, C₁-C₆-heteroalkyl, C₁-C₆-haloalkyl, cycloalkyl, heterocyclyl, aryl, C₁-C₆ alkylene-aryl, C₁-C₆ alkenylene-aryl, C₁-C₆ alkylene-heteroaryl, heteroaryl, halo, cyano, oxo, —OR^{A}, —NR^{B}R^{C}, —NR^{B}C(O)R^{D}, —NO₂, —C(O)NR^{B}R^{C}, —C(O)R^{D}, —C(O)OR^{D}, or —S(O)ₓR^{D}, wherein each alkyl, alkylene, alkenyl, alkynyl, heteroalkyl, haloalkyl, cycloalkyl, heterocyclyl, aryl, and heteroaryl is optionally substituted with one or more R⁶; or two R¹ groups, together with the atoms to which they are attached, form a 3-7-membered cycloalkyl, heterocyclyl, aryl, or heteroaryl, wherein each cycloalkyl, heterocyclyl, aryl, and heteroaryl is optionally substituted with one or more R⁶; R² is hydrogen, halo, cyano, C₁-C₆-alkyl, C₂-C₆-alkenyl, C₂-C₆-alkynyl, C₁-C₆-heteroalkyl, or C₁-C₆-haloalkyl; each R³ is independently hydrogen, C₁-C₆-alkyl, or C₁-C₆-haloalkyl; each R⁴ is independently C₁-C₆-alkyl, C₁-C₆-heteroalkyl, C₁-C₆-haloalkyl, cycloalkyl, halo, cyano, oxo, —OR^{A}, —NR^{B}R^{C}, —C(O)R^{D}, or —C(O)OR^{D}; each R⁶ is independently C₁-C₆-alkyl, C₂-C₆-alkenyl, C₂-C₆-alkynyl, C₁-C₆-heteroalkyl, C₁-C₆-haloalkyl, cycloalkyl, heterocyclyl, aryl, heteroaryl, halo, cyano, oxo, —OR^{A}, —NR^{B}R^{C}, NR^{B}C(O)R^{D}, —NO₂, —C(O)NR^{B}R^{C}, —C(O)R^{D}, —C(O)OR^{D}, or —S(O)ₓR^{D}, wherein each of alkyl, alkenyl, alkynyl, heteroalkyl, haloalkyl, cycloalkyl, heterocyclyl, aryl, and heteroaryl is optionally substituted with one or more R⁷; each R^{A} is independently hydrogen, C₁-C₆ alkyl, C₁-C₆ haloalkyl, aryl, heteroaryl, C₁-C₆ alkylene-aryl, C₁-C₆ alkylene-heteroaryl, —C(O)R^{D}, or —S(O)ₓR^{D}; each of R^{B} and R^{C} is independently hydrogen, C₁-C₆ alkyl, C₁-C₆ heteroalkyl, cycloalkyl, heterocyclyl, or —OR^{A}; or R^{B} and R^{C} together with the atom to which they are attached form a 3-7-membered heterocyclyl ring optionally substituted with one or more R⁸; each R^{D} is independently hydrogen, C₁-C₆ alkyl, C₂-C₆ alkenyl, C₂-C₆ alkynyl, C₁-C₆ heteroalkyl, C₁-C₆ haloalkyl, cycloalkyl, heterocyclyl, aryl, heteroaryl, C₁-C₆ alkylene-aryl, or C₁-C₆ alkylene-heteroaryl; each R⁷ is independently C₁-C₆ alkyl, C₁-C₆ heteroalkyl, C₁-C₆ haloalkyl, cycloalkyl, heterocyclyl, aryl, heteroaryl, halo, cyano, oxo, or —OR^{A}; each R⁸ is C₁-C₆-alkyl, halo, cyano, oxo, or —OR^{A1}; each R^{A1} is hydrogen or C₁-C₆-alkyl; and x is 0, 1, or 2.

In some embodiments, the compound of Formula (II) is a compound of Formula (II-c):

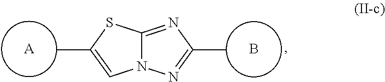

or a pharmaceutically acceptable salt, solvate, hydrate, tautomer, or stereoisomer thereof, wherein A and B are each independently cycloalkyl, heterocyclyl, aryl, or heteroaryl, each of which is optionally substituted with one or more R¹; each R¹ is independently hydrogen, C₁-C₆-alkyl, C₂-C₆-alkenyl, C₂-C₆-alkynyl, C₁-C₆-heteroalkyl, C₁-C₆-haloalkyl, cycloalkyl, heterocyclyl, aryl, C₁-C₆ alkylene-aryl, C₁-C₆ alkenylene-aryl, C₁-C₆ alkylene-heteroaryl, heteroaryl, halo, cyano, oxo, —OR^{A}, —NR^{B}R^{C}, —NR^{B}C(O)R^{D}, —NO₂, —C(O)NR^{B}R^{C}, —C(O)R^{D}, —C(O)OR^{D}, or —S(O)ₓR^{D}, wherein each alkyl, alkylene, alkenyl, alkynyl, heteroalkyl, haloalkyl, cycloalkyl, heterocyclyl, aryl, and heteroaryl is optionally substituted with one or more R⁶; or two R¹ groups, together with the atoms to which they are attached, form a 3-7-membered cycloalkyl, heterocyclyl, aryl, or heteroaryl, wherein each cycloalkyl, heterocyclyl, aryl, and heteroaryl is optionally substituted with one or more R⁶; each R⁶ is independently C₁-C₆-alkyl, C₂-C₆-alkenyl, C₂-C₆-alkynyl, C₁-C₆-heteroalkyl, C₁-C₆-haloalkyl, cycloalkyl, heterocyclyl, aryl, heteroaryl, halo, cyano, oxo, —OR^{A}, —NR^{B}R^{C}, —N^{B}C(O)R^{D}, —NO₂, —C(O)NR^{B}R^{C}, —C(O)R^{D}, —C(O)OR^{D}, or —S(O)ₓR^{D}, wherein each of alkyl, alkenyl, alkynyl, heteroalkyl, haloalkyl, cycloalkyl, heterocyclyl, aryl, and heteroaryl is optionally substituted with one or more R⁷; each R^{A} is independently hydrogen, C₁-C₆ alkyl, C₁-C₆ haloalkyl, aryl, heteroaryl, C₁-C₆ alkylene-aryl, C₁-C₆ alkylene-heteroaryl, —C(O)R^{D}, or —S(O)ₓR^{D}; each of R^{B} and R^{C} is independently hydrogen, C₁-C₆ alkyl, C₁-C₆ heteroalkyl, cycloalkyl, heterocyclyl, or —OR^{A}; or R^{B} and R^{C} together with the atom to which they are attached form a 3-7-membered heterocyclyl ring optionally substituted with one or more R⁸; each R^{D} is independently hydrogen, C₁-C₆ alkyl, C₂-C₆ alkenyl, C₂-C₆ alkynyl, C₁-C₆ heteroalkyl, C₁-C₆ haloalkyl, cycloalkyl, heterocyclyl, aryl, heteroaryl, C₁-C₆ alkylene-aryl, or C₁-C₆ alkylene-heteroaryl; each R⁷ is independently C₁-C₆ alkyl, C₁-C₆ heteroalkyl, C₁-C₆ haloalkyl, cycloalkyl, heterocyclyl, aryl, heteroaryl, halo, cyano, oxo, or —OR^{A}; each R⁸ is C₁-C₆-alkyl, halo, cyano, oxo, or —OR^{A1}; each R^{A1} is hydrogen or C₁-C₆-alkyl; and x is 0, 1, or 2.

In some embodiments, the compound of Formula (II) is a compound of Formula (II-d):

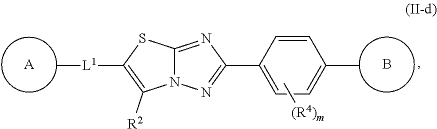

or a pharmaceutically acceptable salt, solvate, hydrate, tautomer, or stereoisomer thereof, wherein A and B are each independently cycloalkyl, heterocyclyl, aryl, or heteroaryl, each of which is optionally substituted with one or more R¹; L¹ is absent, C₁-C₆-alkylene, C₁-C₆-heteroalkylene, —O—, —C(O)—, —N(R³)—, —N(R³)C(O)—, or C(O)N(R³)—, wherein each alkylene and heteroalkylene is optionally substituted with one or more R⁴; each R¹ is independently hydrogen, C₁-C₆-alkyl, C₂-C₆-alkenyl, C₂-C₆-alkynyl, C₁-C₆-heteroalkyl, C₁-C₆-haloalkyl, cycloalkyl, heterocyclyl, aryl, C₁-C₆ alkylene-aryl, C₁-C₆ alkenylene-aryl, C₁-C₆ alkylene-heteroaryl, heteroaryl, halo, cyano, oxo, —OR^{A}, —NR^{B}R^{C}, —N^{B}C(O)R^{D}, —NO₂, —C(O)NR^{B}R^{C}, —C(O)R^{D}, —C(O)OR^{D}, or —S(O)ₓR^{D}, wherein each alkyl, alkylene, alkenyl, alkynyl, heteroalkyl, haloalkyl, cycloalkyl, heterocyclyl, aryl, and heteroaryl is optionally substituted with one or more R⁶; or two R¹ groups, together with the atoms to which they are attached, form a 3-7-membered cycloalkyl, heterocyclyl, aryl, or heteroaryl, wherein each cycloalkyl, heterocyclyl, aryl, and heteroaryl is optionally substituted with one or more R⁶; R² is hydrogen, halo, cyano, C₁-C₆-alkyl, C₂-C₆-alkenyl, C₂-C₆-alkynyl, C₁-C₆-heteroalkyl, or C₁-C₆-haloalkyl; each R³ is independently hydrogen, C₁-C₆-alkyl, or C₁-C₆-haloalkyl; each R⁴ is independently C₁-C₆-alkyl, C₁-C₆-heteroalkyl, C₁-C₆-haloalkyl, cycloalkyl, halo, cyano, oxo, —OR^{A}, —NR^{B}R^{C}, —C(O)R^{D}, or —C(O)OR^{D}; each R⁶ is independently C₁-C₆-alkyl, C₂-C₆-alkenyl, C₂-C₆-alkynyl, C₁-C₆-heteroalkyl, C₁-C₆-haloalkyl, cycloalkyl, heterocyclyl, aryl, heteroaryl, halo, cyano, oxo, —OR^{A}, —NR^{B}R^{C}, —NR^{B}C(O)R^{D}, —NO₂, —C(O)NR^{B}R^{C}, —C(O)R^{D}, —C(O)OR^{D}, or —S(O)ₓR^{D}, wherein each of alkyl, alkenyl, alkynyl, heteroalkyl, haloalkyl, cycloalkyl, heterocyclyl, aryl, and heteroaryl is optionally substituted with one or more R⁷; each R^{A} is independently hydrogen, C₁-C₆ alkyl, C₁-C₆ haloalkyl, aryl, heteroaryl, C₁-C₆ alkylene-aryl, C₁-C₆ alkylene-heteroaryl, —C(O)R^{D}, or —S(O)ₓR^{D}; each of R^{B} and R^{C} is independently hydrogen, C₁-C₆ alkyl, C₁-C₆ heteroalkyl, cycloalkyl, heterocyclyl, or —OR^{A}; or R^{B} and R^{C} together with the atom to which they are attached form a 3-7-membered heterocyclyl ring optionally substituted with one or more R⁸; each R^{D} is independently hydrogen, C₁-C₆ alkyl, C₂-C₆ alkenyl, C₂-C₆ alkynyl, C₁-C₆ heteroalkyl, C₁-C₆ haloalkyl, cycloalkyl, heterocyclyl, aryl, heteroaryl, C₁-C₆ alkylene-aryl, or C₁-C₆ alkylene-heteroaryl; each R⁷ is independently C₁-C₆ alkyl, C₁-C₆ heteroalkyl, C₁-C₆ haloalkyl, cycloalkyl, heterocyclyl, aryl, heteroaryl, halo, cyano, oxo, or —OR^{A}; each R⁸ is C₁-C₆-alkyl, halo, cyano, oxo, or —OR^{A}1; each R^{A1} is hydrogen or C₁-C₆-alkyl; m is 0, 1, 2, 3, or 4; and x is 0, 1, or 2.

In some embodiments, the compound of Formula (II) is selected from a compound in Table 2, or a pharmaceutically acceptable salt, solvate, hydrate, tautomer, or stereoisomer thereof.

TABLE 2
Exemplary compounds of Formula (II)

| Compound No. | Structure |
|---|---|
| 102 | 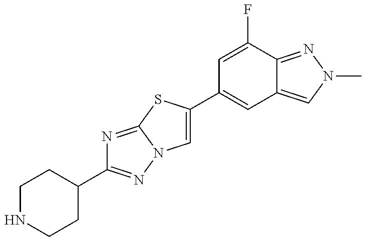 [SMILES: Cn1cc2cc(-c3cn4nc(C5CCNCC5)nc4s3)cc(F)c2n1] |
| 103 | 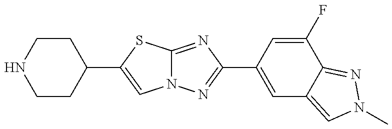 [SMILES: Cn1cc2cc(-c3nc4sc(C5CCNCC5)cn4n3)cc(F)c2n1] |
| 107 | 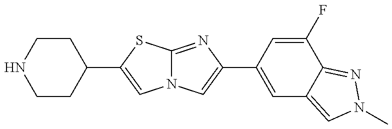 [SMILES: Cn1cc2cc(-c3cn4cc(C5CCNCC5)sc4n3)cc(F)c2n1] |
| 109 | 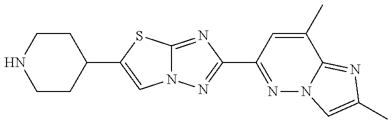 [SMILES: Cc1cn2nc(-c3nc4sc(C5CCNCC5)cn4n3)cc(C)c2n1] |
| 113 | 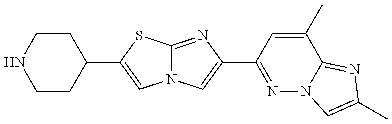 [SMILES: Cc1cn2nc(-c3cn4cc(C5CCNCC5)sc4n3)cc(C)c2n1] |
| 114 | 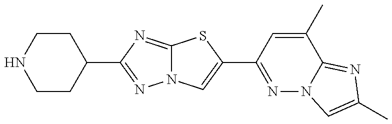 [SMILES: Cc1cn2nc(-c3cn4nc(C5CCNCC5)nc4s3)cc(C)c2n1] |
| 115 | 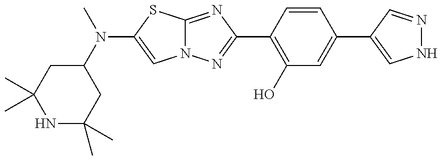 [SMILES: CN(c1cn2nc(-c3ccc(-c4cn[nH]c4)cc3O)nc2s1)C1CC(C)(C)NC(C)(C)C1] |
| 119 | 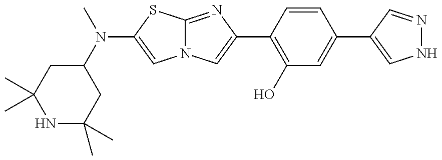 [SMILES: CN(c1cn2cc(-c3ccc(-c4cn[nH]c4)cc3O)nc2s1)C1CC(C)(C)NC(C)(C)C1] |
| 123 | 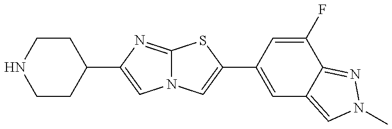 [SMILES: Cn1cc2cc(-c3cn4cc(C5CCNCC5)nc4s3)cc(F)c2n1] |

In some embodiments, for Formula (II), A is bicyclic heteroaryl (e.g., 7-fluoro-2-methyl-2H-indazolyl); B is monocyclic heterocyclyl (e.g., piperidinyl); L¹ and L² are each absent; Y is N; and R² is hydrogen. In some embodiments, the compound of Formula (II), (II-b), and (II-c) is Compound 102, or a pharmaceutically acceptable salt, solvate, hydrate, tautomer, or stereoisomer thereof.

In some embodiments, for Formula (II), A is monocyclic heterocyclyl (e.g., piperidinyl); B is bicyclic heteroaryl (e.g., 7-fluoro-2-methyl-2H-indazolyl); L¹ and L² are each absent; Y is N; and R² is hydrogen. In some embodiments, the compound of Formula (II), (II-b), and (II-c) is Compound 103, or a pharmaceutically acceptable salt, solvate, hydrate, tautomer, or stereoisomer thereof.

In some embodiments, for Formula (II), A is monocyclic heterocyclyl (e.g., piperidinyl); B is bicyclic heteroaryl (e.g., 7-fluoro-2-methyl-2H-indazolyl); L¹ and L² are each absent; Y is C(R⁵) (e.g., CH); and R² is hydrogen. In some embodiments, the compound of Formula (II) and (II-a) is Compound 107, or a pharmaceutically acceptable salt, solvate, hydrate, tautomer, or stereoisomer thereof.

In some embodiments, for Formula (II), A is monocyclic heterocyclyl (e.g., piperidinyl); B is bicyclic heteroaryl (e.g., 2,8-dimethylimidazo[1,2-b]pyridazinyl); L¹ and L² are each absent; Y is N; and R² is hydrogen. In some embodiments, the compound of Formula (II), (II-b), and (II-c) is Compound 109, or a pharmaceutically acceptable salt, solvate, hydrate, tautomer, or stereoisomer thereof.

In some embodiments, for Formula (II), A is monocyclic heterocyclyl (e.g., piperidinyl); B is bicyclic heteroaryl (e.g., 2,8-dimethylimidazo[1,2-b]pyridazinyl); L¹ and L² are each absent; Y is C(R⁵) (e.g., CH); and R² is hydrogen. In some embodiments, the compound of Formula (II) and (II-a) is Compound 113, or a pharmaceutically acceptable salt, solvate, hydrate, tautomer, or stereoisomer thereof.

In some embodiments, for Formula (II), A is bicyclic heteroaryl (e.g., 2,8-dimethylimidazo[1,2-b]pyridazinyl); B is monocyclic heterocyclyl (e.g., piperidinyl); L¹ and L² are each absent; Y is N; and R² is hydrogen. In some embodiments, the compound of Formula (II), (II-b), and (II-c) is Compound 114, or a pharmaceutically acceptable salt, solvate, hydrate, tautomer, or stereoisomer thereof.

In some embodiments, for Formula (II), A is monocyclic heterocyclyl (e.g., 2,2,6,6-tetramethylpiperidinyl); B is monocyclic heteroaryl (e.g., pyrazolyl); L¹ is —NR³— (e.g., —N(CH₃)—); L² is C₆-C₁₂ arylene substituted with one R⁴; Y is N; R² is hydrogen; and R⁴ is —OR^{A} (e.g., —OH). In some embodiments, the compound of Formula (II), (II-b), and (II-c) is Compound 115, or a pharmaceutically acceptable salt, solvate, hydrate, tautomer, or stereoisomer thereof.

In some embodiments, for Formula (II), A is monocyclic heterocyclyl (e.g., 2,2,6,6-tetramethylpiperidinyl); B is monocyclic heteroaryl (e.g., pyrazolyl); L¹ is —NR³— (e.g., —N(CH₃)—); L² is C₆-C₁₂ arylene substituted with one R⁴; Y is C(R⁵) (e.g., CH); R² is hydrogen; and R⁴ is —OR^{A} (e.g., —OH). In some embodiments, the compound of Formula (II) and (II-a) is Compound 119, or a pharmaceutically acceptable salt, solvate, hydrate, tautomer, or stereoisomer thereof.

In some embodiments, for Formula (II), A is bicyclic heteroaryl (e.g., 7-fluoro-2-methyl-2H-indazolyl); B is monocyclic heterocyclyl (e.g., piperidinyl); L¹ and L² are each absent; Y is C(R⁵) (e.g., CH); and R² is hydrogen. In some embodiments, the compound of Formula (II) and (II-a) is Compound 123, or a pharmaceutically acceptable salt, solvate, hydrate, tautomer, or stereoisomer thereof.

In some embodiments, the present disclosure features a compound of Formula (III-a):

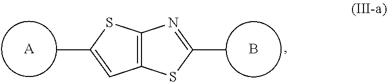

or a pharmaceutically acceptable salt, solvate, hydrate, tautomer, or stereoisomer thereof, wherein A and B are each independently cycloalkyl, heterocyclyl, aryl, or heteroaryl, each of which is optionally substituted with one or more R¹; each R¹ is independently hydrogen, C₁-C₆-alkyl, C₂-C₆-alkenyl, C₂-C₆-alkynyl, C₁-C₆-heteroalkyl, C₁-C₆-haloalkyl, cycloalkyl, heterocyclyl, aryl, C₁-C₆ alkylene-aryl, C₁-C₆ alkenylene-aryl, C₁-C₆ alkylene-heteroaryl, heteroaryl, halo, cyano, oxo, —OR^{A}, —NR^{B}R^{C}, —NR^{B}C(O)R^{D}, —NO₂, —C(O)NR^{B}R^{C}, —C(O)R^{D}, —C(O)OR^{D}, or —S(O)ₓR^{D}, wherein each alkyl, alkylene, alkenyl, alkynyl, heteroalkyl, haloalkyl, cycloalkyl, heterocyclyl, aryl, and heteroaryl is optionally substituted with one or more R⁶; or two R¹ groups, together with the atoms to which they are attached, form a 3-7-membered cycloalkyl, heterocyclyl, aryl, or heteroaryl, wherein each cycloalkyl, heterocyclyl, aryl, and heteroaryl is optionally substituted with one or more R⁶; R² is hydrogen, halo, cyano, C₁-C₆-alkyl, C₂-C₆-alkenyl, C₂-C₆-alkynyl, C₁-C₆-heteroalkyl, or C₁-C₆-haloalkyl; each R⁶ is independently C₁-C₆-alkyl, C₂-C₆-alkenyl, C₂-C₆-alkynyl, C₁-C₆-heteroalkyl, C₁-C₆-haloalkyl, cycloalkyl, heterocyclyl, aryl, heteroaryl, halo, cyano, oxo, —OR^{A}, —NR^{B}R^{C}, —N^{B}C(O)R^{D}, —NO₂, —C(O)NR^{B}R^{C}, —C(O)R^{D}, —C(O)OR^{D}, or —S(O)ₓR^{D}, wherein each of alkyl, alkenyl, alkynyl, heteroalkyl, haloalkyl, cycloalkyl, heterocyclyl, aryl, and heteroaryl is optionally substituted with one or more R⁷; each R^{A} is independently hydrogen, C₁-C₆ alkyl, C₁-C₆ haloalkyl, aryl, heteroaryl, C₁-C₆ alkylene-aryl, C₁-C₆ alkylene-heteroaryl, —C(O)R^{D}, or —S(O)ₓR^{D}; each of R^{B} and R^{C} is independently hydrogen, C₁-C₆ alkyl, C₁-C₆ heteroalkyl, cycloalkyl, heterocyclyl, or —OR^{A}; or R^{B} and R^{C} together with the atom to which they are attached form a 3-7-membered heterocyclyl ring optionally substituted with one or more R⁸; each R is independently hydrogen, C₁-C₆ alkyl, C₂-C₆ alkenyl, C₂-C₆ alkynyl, C₁-C₆ heteroalkyl, C₁-C₆ haloalkyl, cycloalkyl, heterocyclyl, aryl, heteroaryl, C₁-C₆ alkylene-aryl, or C₁-C₆ alkylene-heteroaryl; each R⁷ is independently C₁-C₆ alkyl, C₁-C₆ heteroalkyl, C₁-C₆ haloalkyl, cycloalkyl, heterocyclyl, aryl, heteroaryl, halo, cyano, oxo, or —OR^{A}; each R⁸ is C₁-C₆-alkyl, halo, cyano, oxo, or —OR^{A1}; each R^{A1} is hydrogen or C₁-C₆-alkyl; and x is 0, 1, or 2.

In some embodiments, the compound of Formula (III) is a compound of Formula (III-b):

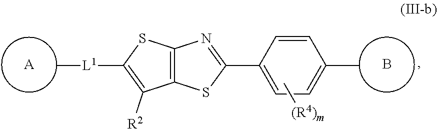

or a pharmaceutically acceptable salt, solvate, hydrate, tautomer, or stereoisomer thereof, wherein A and B are each independently cycloalkyl, heterocyclyl, aryl, or heteroaryl, each of which is optionally substituted with one or more R¹; L¹ is absent, C₁-C₆-alkylene, C₁-C₆-heteroalkylene, —O—, —C(O)—, —N(R³)—, —N(R³)C(O)—, or C(O)N(R³)—, wherein each alkylene and heteroalkylene is optionally substituted with one or more R⁴; each R¹ is independently hydrogen, C₁-C₆-alkyl, C₂-C₆-alkenyl, C₂-C₆-alkynyl, C₁-C₆-heteroalkyl, C₁-C₆-haloalkyl, cycloalkyl, heterocyclyl, aryl, C₁-C₆ alkylene-aryl, C₁-C₆ alkenylene-aryl, C₁-C₆ alkylene-heteroaryl, heteroaryl, halo, cyano, oxo, —OR^{A}, —NR^{B}R^{C}, —N^{B}C(O)R^{D}, —NO₂, —C(O)NR^{B}R^{C}, —C(O)R^{D}, —C(O)OR^{D}, or —S(O)ₓR^{D}, wherein each alkyl, alkylene, alkenyl, alkynyl, heteroalkyl, haloalkyl, cycloalkyl, heterocyclyl, aryl, and heteroaryl is optionally substituted with one or more R⁶; or two R¹ groups, together with the atoms to which they are attached, form a 3-7-membered cycloalkyl, heterocyclyl, aryl, or heteroaryl, wherein each cycloalkyl, heterocyclyl, aryl, and heteroaryl is optionally substituted with one or more R⁶; R² is hydrogen, halo, cyano, C₁-C₆-alkyl, C₂-C₆-alkenyl, C₂-C₆-alkynyl, C₁-C₆-heteroalkyl, or C₁-C₆-haloalkyl; each R³ is independently hydrogen, C₁-C₆-alkyl, or C₁-C₆-haloalkyl; each R⁴ is independently C₁-C₆-alkyl, C₁-C₆-heteroalkyl, C₁-C₆-haloalkyl, cycloalkyl, halo, cyano, oxo, —OR^{A}, —NR^{B}R^{C}, —C(O)R^{D}, or —C(O)OR^{D}; each R⁶ is independently C₁-C₆-alkyl, C₂-C₆-alkenyl, C₂-C₆-alkynyl, C₁-C₆-heteroalkyl, C₁-C₆-haloalkyl, cycloalkyl, heterocyclyl, aryl, heteroaryl, halo, cyano, oxo, —OR^{A}, —NR^{B}R^{C}, —NR^{B}C(O)R^{D}, —NO₂, —C(O)NR^{B}R^{C}, —C(O)R^{D}, —C(O)OR^{D}, or —S(O)ₓR^{D}, wherein each of alkyl, alkenyl, alkynyl, heteroalkyl, haloalkyl, cycloalkyl, heterocyclyl, aryl, and heteroaryl is optionally substituted with one or more R⁷; each R^{A} is independently hydrogen, C₁-C₆ alkyl, C₁-C₆ haloalkyl, aryl, heteroaryl, C₁-C₆ alkylene-aryl, C₁-C₆ alkylene-heteroaryl, —C(O)R^{D}, or —S(O)ₓR^{D}; each of R^{B} and R^{C} is independently hydrogen, C₁-C₆ alkyl, C₁-C₆ heteroalkyl, cycloalkyl, heterocyclyl, or —OR^{A}; or R^{B} and R^{C} together with the atom to which they are attached form a 3-7-membered heterocyclyl ring optionally substituted with one or more R⁸; each R^{D} is independently hydrogen, C₁-C₆ alkyl, C₂-C₆ alkenyl, C₂-C₆ alkynyl, C₁-C₆ heteroalkyl, C₁-C₆ haloalkyl, cycloalkyl, heterocyclyl, aryl, heteroaryl, C₁-C₆ alkylene-aryl, or C₁-C₆ alkylene-heteroaryl; each R⁷ is independently C₁-C₆ alkyl, C₁-C₆ heteroalkyl, C₁-C₆ haloalkyl, cycloalkyl, heterocyclyl, aryl, heteroaryl, halo, cyano, oxo, or —OR^{A}; each R⁸ is C₁-C₆-alkyl, halo, cyano, oxo, or —OR^{A1}; each R^{A1} is hydrogen or C₁-C₆-alkyl; m is 0, 1, 2, 3, or 4; and x is 0, 1, or 2.

In some embodiments, the present disclosure features a compound of Formula (III-c):

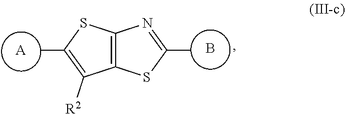

or a pharmaceutically acceptable salt, solvate, hydrate, tautomer, or stereoisomer thereof, wherein A and B are each independently cycloalkyl, heterocyclyl, aryl, or heteroaryl, each of which is optionally substituted with one or more R¹; each R¹ is independently hydrogen, C₁-C₆-alkyl, C₂-C₆-alkenyl, C₂-C₆-alkynyl, C₁-C₆-heteroalkyl, C₁-C₆-haloalkyl, cycloalkyl, heterocyclyl, aryl, C₁-C₆ alkylene-aryl, C₁-C₆ alkenylene-aryl, C₁-C₆ alkylene-heteroaryl, heteroaryl, halo, cyano, oxo, —OR^{A}, —NR^{B}R^{C}, —NR^{B}C(O)R^{D}, —NO₂, —C(O)NR^{B}R^{C}, —C(O)R^{D}, —C(O)OR^{D}, or —S(O)ₓR^{D}, wherein each alkyl, alkylene, alkenyl, alkynyl, heteroalkyl, haloalkyl, cycloalkyl, heterocyclyl, aryl, and heteroaryl is optionally substituted with one or more R⁶; or two R¹ groups, together with the atoms to which they are attached, form a 3-7-membered cycloalkyl, heterocyclyl, aryl, or heteroaryl, wherein each cycloalkyl, heterocyclyl, aryl, and heteroaryl is optionally substituted with one or more R⁶; R² is hydrogen, halo, cyano, C₁-C₆-alkyl, C₂-C₆-alkenyl, C₂-C₆-alkynyl, C₁-C₆-heteroalkyl, or C₁-C₆-haloalkyl; each R⁶ is independently C₁-C₆-alkyl, C₂-C₆-alkenyl, C₂-C₆-alkynyl, C₁-C₆-heteroalkyl, C₁-C₆-haloalkyl, cycloalkyl, heterocyclyl, aryl, heteroaryl, halo, cyano, oxo, —OR^{A}, —NR^{B}R^{C}, —NR^{B}C(O)R^{D}, —NO₂, —C(O)NR^{B}R^{C}, —C(O)R^{D}, —C(O)OR^{D}, or —S(O)ₓR^{D}, wherein each of alkyl, alkenyl, alkynyl, heteroalkyl, haloalkyl, cycloalkyl, heterocyclyl, aryl, and heteroaryl is optionally substituted with one or more R⁷; each R^{A} is independently hydrogen, C₁-C₆ alkyl, C₁-C₆ haloalkyl, aryl, heteroaryl, C₁-C₆ alkylene-aryl, C₁-C₆ alkylene-heteroaryl, —C(O)R^{D}, or —S(O)ₓR^{D}; each of R^{B} and R^{C} is independently hydrogen, C₁-C₆ alkyl, C₁-C₆ heteroalkyl, cycloalkyl, heterocyclyl, or —OR^{A}; or R^{B} and R^{C} together with the atom to which they are attached form a 3-7-membered heterocyclyl ring optionally substituted with one or more R⁸; each R is independently hydrogen, C₁-C₆ alkyl, C₂-C₆ alkenyl, C₂-C₆ alkynyl, C₁-C₆ heteroalkyl, C₁-C₆ haloalkyl, cycloalkyl, heterocyclyl, aryl, heteroaryl, C₁-C₆ alkylene-aryl, or C₁-C₆ alkylene-heteroaryl; each R⁷ is independently C₁-C₆ alkyl, C₁-C₆ heteroalkyl, C₁-C₆ haloalkyl, cycloalkyl, heterocyclyl, aryl, heteroaryl, halo, cyano, oxo, or —OR^{A}; each R⁸ is C₁-C₆-alkyl, halo, cyano, oxo, or —OR^{A1}; each R^{A1} is hydrogen or C₁-C₆-alkyl; and x is 0, 1, or 2.

In some embodiments, the compound of Formula (III) is selected from a compound in Table 3, or a pharmaceutically acceptable salt, solvate, hydrate, tautomer, or stereoisomer thereof.

TABLE 3
Exemplary compounds of Formula (III)

| Compound No. | Structure |
|---|---|
| 106 | 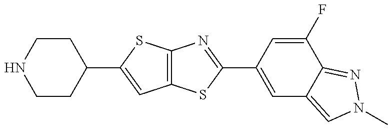 [SMILES: Cn1cc2cc(-c3nc4sc(C5CCNCC5)cc4s3)cc(F)c2n1] |
| 112 | 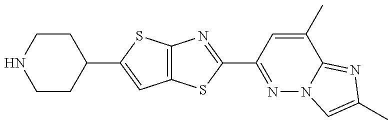 [SMILES: Cc1cn2nc(-c3nc4sc(C5CCNCC5)cc4s3)cc(C)c2n1] |
| 118 | 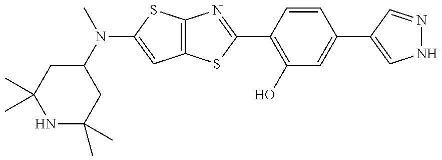 [SMILES: CN(c1cc2sc(-c3ccc(-c4cn[nH]c4)cc3O)nc2s1)C1CC(C)(C)NC(C)(C)C1] |
| 124 | 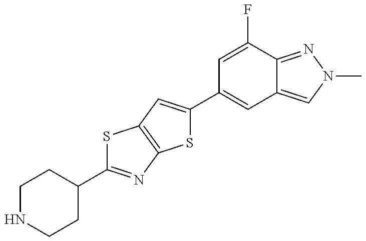 [SMILES: Cn1cc2cc(-c3cc4sc(C5CCNCC5)nc4s3)cc(F)c2n1] |
| 125 | 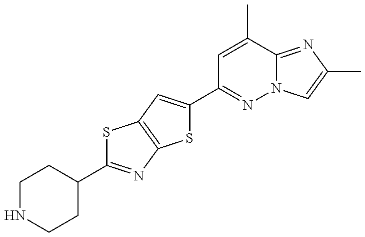 [SMILES: Cc1cn2nc(-c3cc4sc(C5CCNCC5)nc4s3)cc(C)c2n1] |
| 126 | 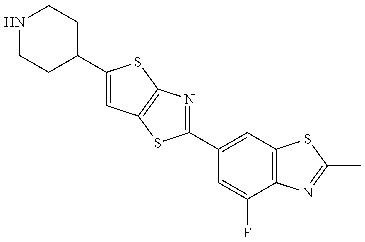 [SMILES: Cc1nc2c(F)cc(-c3nc4sc(C5CCNCC5)cc4s3)cc2s1] |
| 127 | 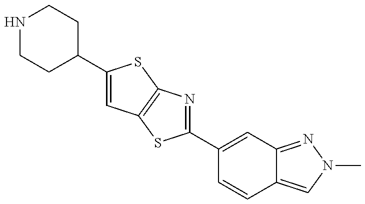 [SMILES: Cn1cc2ccc(-c3nc4sc(C5CCNCC5)cc4s3)cc2n1] |
| 128 | 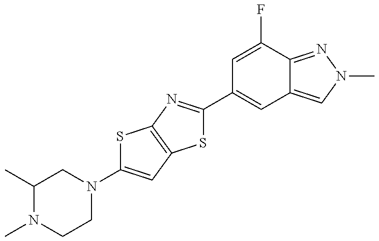 |
| 129 | 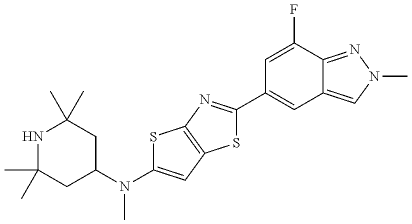 |
| 130 | 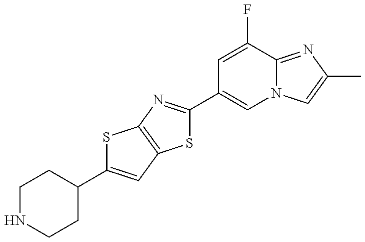 [SMILES: Cc1cn2cc(-c3nc4sc(C5CCNCC5)cc4s3)cc(F)c2n1] |
| 131 | 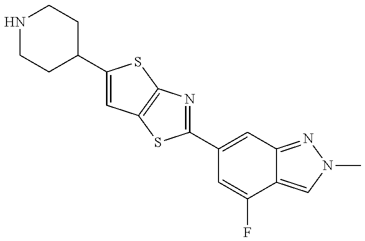 [SMILES: Cn1cc2c(F)cc(-c3nc4sc(C5CCNCC5)cc4s3)cc2n1] |
| 132 | 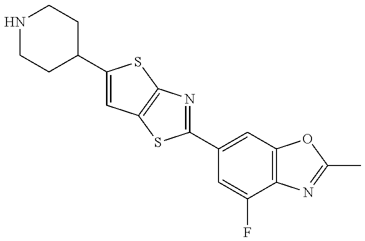 [SMILES: Cc1nc2c(F)cc(-c3nc4sc(C5CCNCC5)cc4s3)cc2o1] |
| 133 | 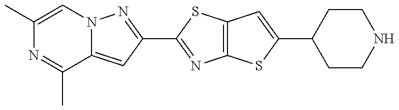 [SMILES: Cc1cn2nc(-c3nc4sc(C5CCNCC5)cc4s3)cc2c(C)n1] |
| 134 | 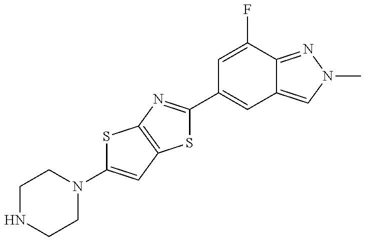 [SMILES: Cn1cc2cc(-c3nc4sc(N5CCNCC5)cc4s3)cc(F)c2n1] |
| 135 | 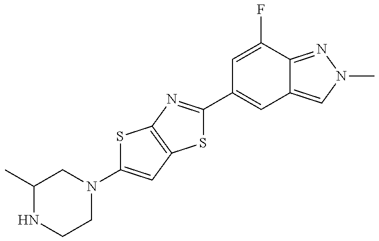 |
| 136 | 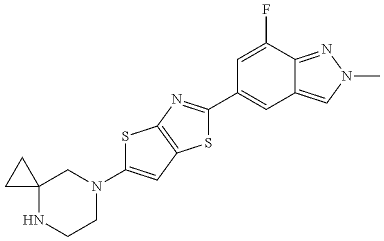 [SMILES: Cn1cc2cc(-c3nc4sc(N5CCNC6(CC6)C5)cc4s3)cc(F)c2n1] |
| 137 | 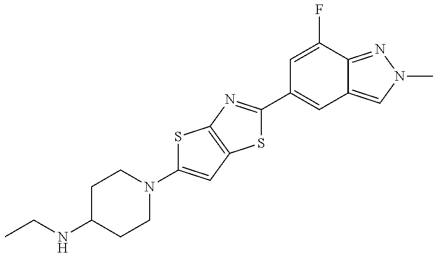 [SMILES: CCNC1CCN(c2cc3sc(-c4cc(F)c5nn(C)cc5c4)nc3s2)CC1] |
| 138 | 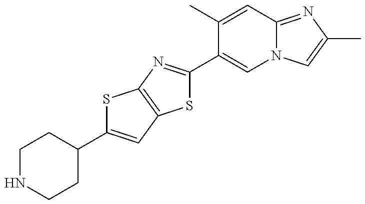 [SMILES: Cc1cn2cc(-c3nc4sc(C5CCNCC5)cc4s3)c(C)cc2n1] |
| 139 | 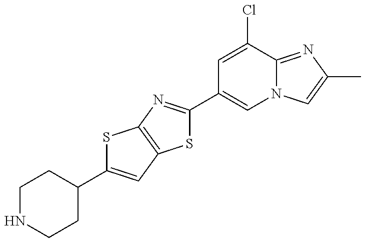 [SMILES: Cc1cn2cc(-c3nc4sc(C5CCNCC5)cc4s3)cc(Cl)c2n1] |
| 140 | 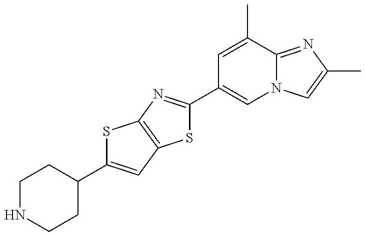 [SMILES: Cc1cn2cc(-c3nc4sc(C5CCNCC5)cc4s3)cc(C)c2n1] |
| 141 | 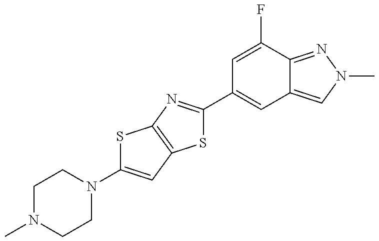 |
| 142 | 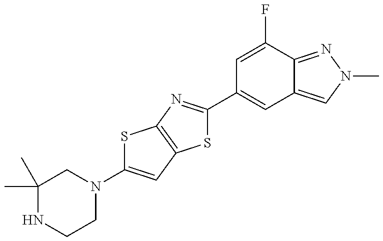 |
| 143 | 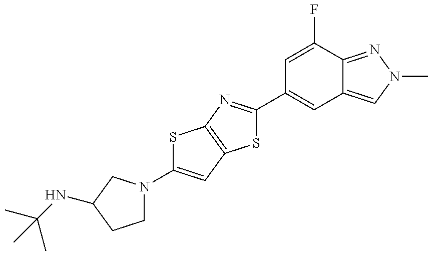 [SMILES: Cn1cc2cc(-c3nc4sc(N5CCC(NC(C)(C)C)C5)cc4s3)cc(F)c2n1] |
| 144 | 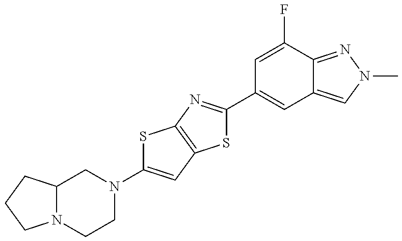 [SMILES: Cn1cc2cc(-c3nc4sc(N5CCN6CCCC6C5)cc4s3)cc(F)c2n1] |
| 145 | 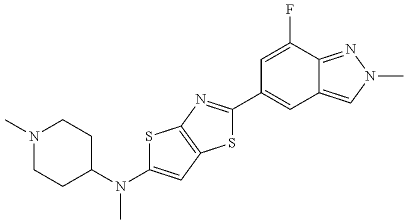 |
| 146 | 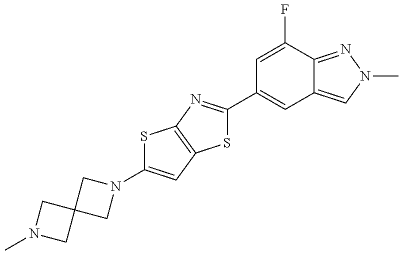 |
| 147 | 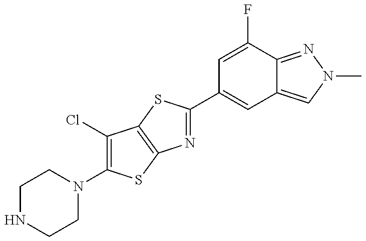 [SMILES: Cn1cc2cc(-c3nc4sc(N5CCNCC5)c(Cl)c4s3)cc(F)c2n1] |
| 151 | 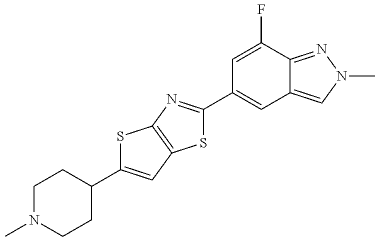 |
| 152 | 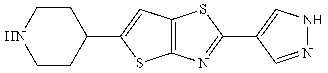 [SMILES: c1n[nH]cc1-c1nc2sc(C3CCNCC3)cc2s1] |
| 153 | 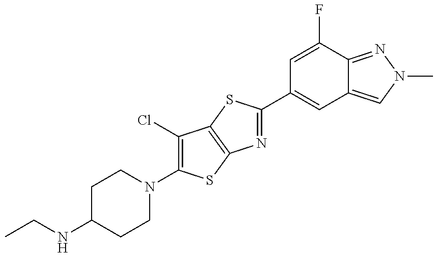 [SMILES: CCNC1CCN(c2sc3nc(-c4cc(F)c5nn(C)cc5c4)sc3c2Cl)CC1] |
| 154 | 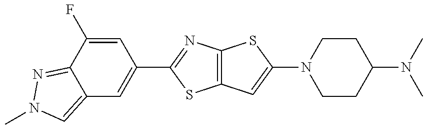 |
| 191 | 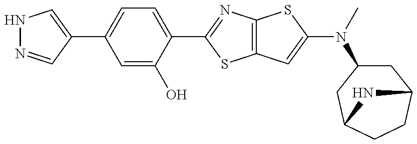 [SMILES: CN(c1cc2sc(-c3ccc(-c4cn[nH]c4)cc3O)nc2s1)[C@@H]1C[C@H]2CC[C@@H](C1)N2] |
| 192 | 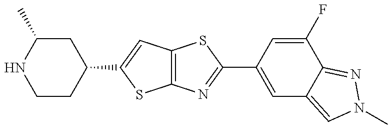 [SMILES: C[C@@H]1C[C@H](c2cc3sc(-c4cc(F)c5nn(C)cc5c4)nc3s2)CCN1] |
| 193 | 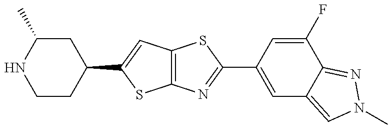 [SMILES: C[C@@H]1C[C@@H](c2cc3sc(-c4cc(F)c5nn(C)cc5c4)nc3s2)CCN1] |
| 194 | 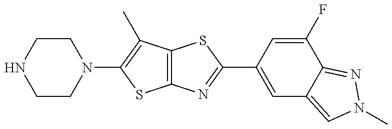 [SMILES: Cc1c(N2CCNCC2)sc2nc(-c3cc(F)c4nn(C)cc4c3)sc12] |
| 195 | 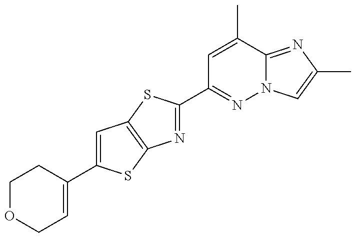 [SMILES: Cc1cn2nc(-c3nc4sc(C5=CCOCC5)cc4s3)cc(C)c2n1] |
| 196 | 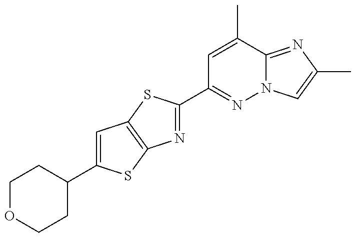 [SMILES: Cc1cn2nc(-c3nc4sc(C5CCOCC5)cc4s3)cc(C)c2n1] |
| 197 | 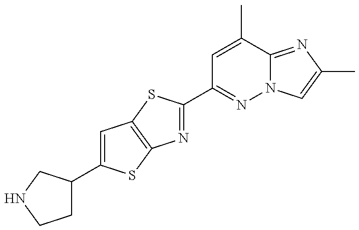 [SMILES: Cc1cn2nc(-c3nc4sc(C5CCNC5)cc4s3)cc(C)c2n1] |
| 198 | 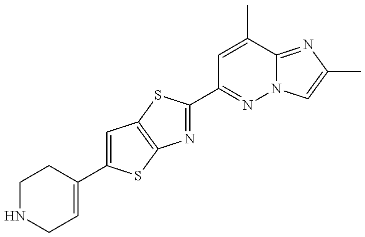 [SMILES: Cc1cn2nc(-c3nc4sc(C5=CCNCC5)cc4s3)cc(C)c2n1] |
| 199 | 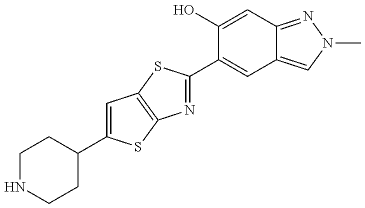 [SMILES: Cn1cc2cc(-c3nc4sc(C5CCNCC5)cc4s3)c(O)cc2n1] |
| 200 | 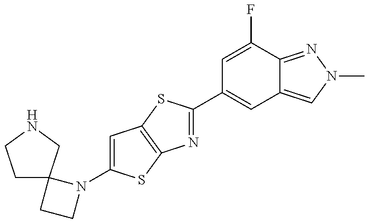 [SMILES: Cn1cc2cc(-c3nc4sc(N5CCC56CCNC6)cc4s3)cc(F)c2n1] |
| 201 | 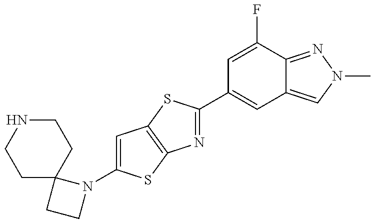 [SMILES: Cn1cc2cc(-c3nc4sc(N5CCC56CCNCC6)cc4s3)cc(F)c2n1] |
| 202 | 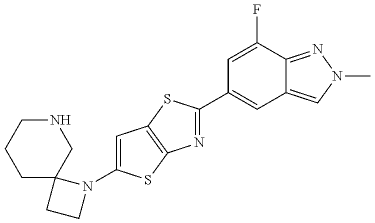 [SMILES: Cn1cc2cc(-c3nc4sc(N5CCC56CCCNC6)cc4s3)cc(F)c2n1] |
| 203 | 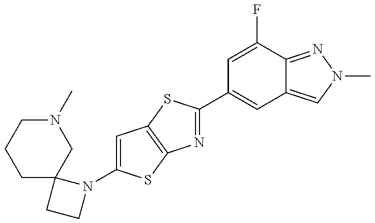 [SMILES: CN1CCCC2(CCN2c2cc3sc(-c4cc(F)c5nn(C)cc5c4)nc3s2)C1] |
| 204 | 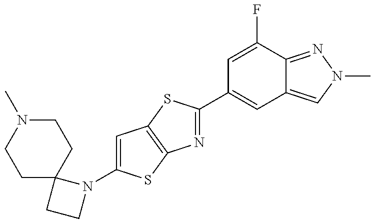 [SMILES: CN1CCC2(CC1)CCN2c1cc2sc(-c3cc(F)c4nn(C)cc4c3)nc2s1] |
| 205 | 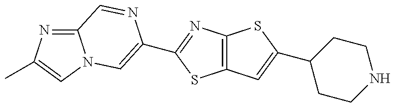 [SMILES: Cc1cn2cc(-c3nc4sc(C5CCNCC5)cc4s3)ncc2n1] |
| 206 | 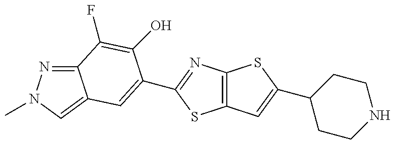 [SMILES: Cn1cc2cc(-c3nc4sc(C5CCNCC5)cc4s3)c(O)c(F)c2n1] |
| 230 | 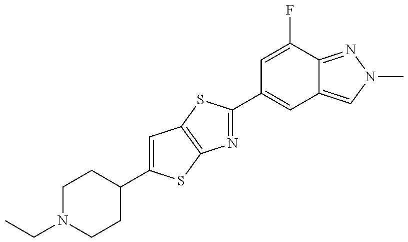 [SMILES: CCN1CCC(c2cc3sc(-c4cc(F)c5nn(C)cc5c4)nc3s2)CC1] |
| 231 | 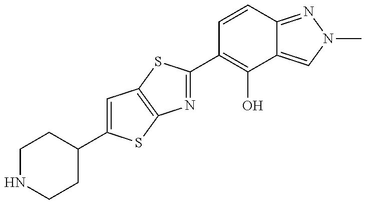 [SMILES: Cn1cc2c(O)c(-c3nc4sc(C5CCNCC5)cc4s3)ccc2n1] |
| 232 | 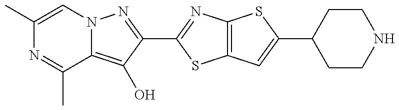 [SMILES: Cc1cn2nc(-c3nc4sc(C5CCNCC5)cc4s3)c(O)c2c(C)n1] |
| 233 | 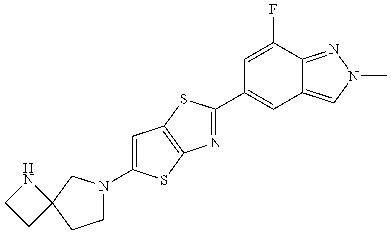 [SMILES: Cn1cc2cc(-c3nc4sc(N5CCC6(CCN6)C5)cc4s3)cc(F)c2n1] |
| 234 | 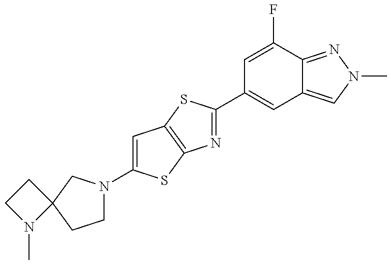 [SMILES: CN1CCC12CCN(c1cc3sc(-c4cc(F)c5nn(C)cc5c4)nc3s1)C2] |
| 235 | 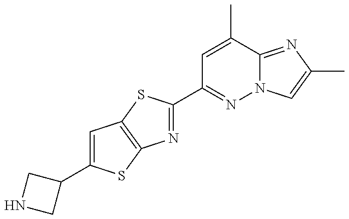 [SMILES: Cc1cn2nc(-c3nc4sc(C5CNC5)cc4s3)cc(C)c2n1] |
| 236 | 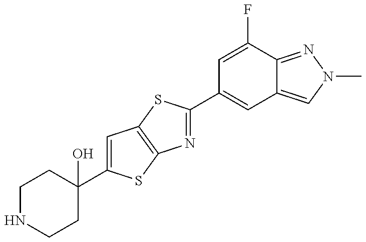 [SMILES: Cn1cc2cc(-c3nc4sc(C5(O)CCNCC5)cc4s3)cc(F)c2n1] |
| 237 | 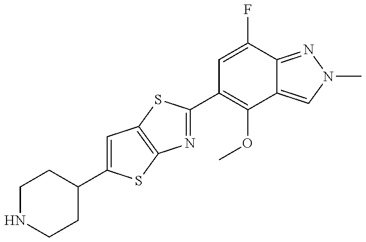 [SMILES: COc1c(-c2nc3sc(C4CCNCC4)cc3s2)cc(F)c2nn(C)cc12] |
| 238 | 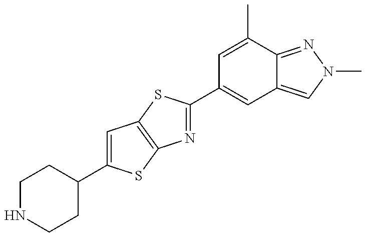 [SMILES: Cc1cc(-c2nc3sc(C4CCNCC4)cc3s2)cc2cn(C)nc12] |
| 239 | 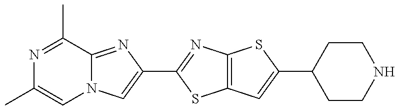 [SMILES: Cc1cn2cc(-c3nc4sc(C5CCNCC5)cc4s3)nc2c(C)n1] |
| 240 | 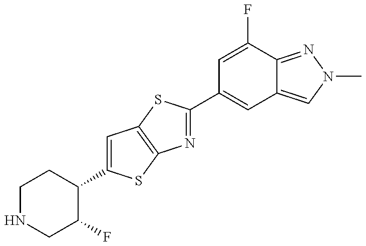 [SMILES: Cn1cc2cc(-c3nc4sc([C@H]5CCNC[C@H]5F)cc4s3)cc(F)c2n1] |
| 241 | 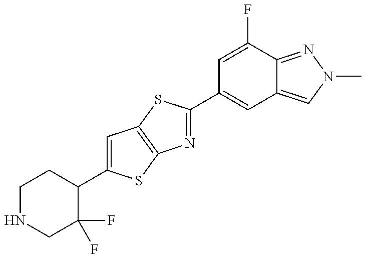 [SMILES: Cn1cc2cc(-c3nc4sc(C5CCNCC5(F)F)cc4s3)cc(F)c2n1] |
| 242 | 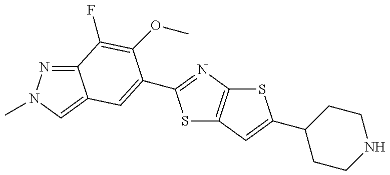 [SMILES: COc1c(-c2nc3sc(C4CCNCC4)cc3s2)cc2cn(C)nc2c1F] |
| 243 | 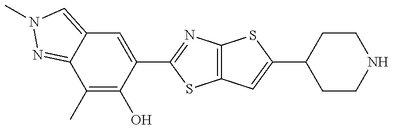 [SMILES: Cc1c(O)c(-c2nc3sc(C4CCNCC4)cc3s2)cc2cn(C)nc12] |
| 247 | 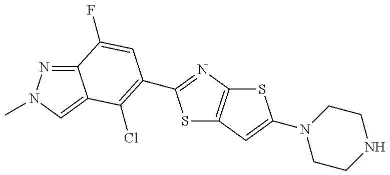 [SMILES: Cn1cc2c(Cl)c(-c3nc4sc(N5CCNCC5)cc4s3)cc(F)c2n1] |
| 257 | 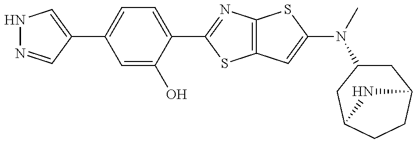 |
| 258 | 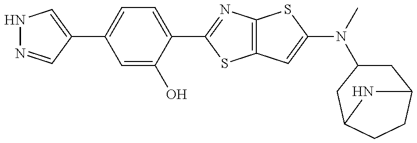 [SMILES: CN(c1cc2sc(-c3ccc(-c4cn[nH]c4)cc3O)nc2s1)C1CC2CCC(C1)N2] |
| 259 | 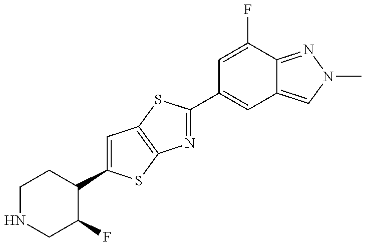 |
| 260 | 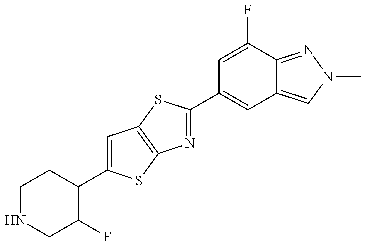 [SMILES: Cn1cc2cc(-c3nc4sc(C5CCNCC5F)cc4s3)cc(F)c2n1] |
| 261 | 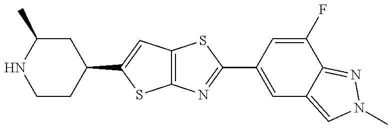 [SMILES: C[C@H]1C[C@@H](c2cc3sc(-c4cc(F)c5nn(C)cc5c4)nc3s2)CCN1] |
| 262 | 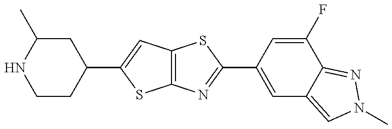 [SMILES: CC1CC(c2cc3sc(-c4cc(F)c5nn(C)cc5c4)nc3s2)CCN1] |
| 263 | 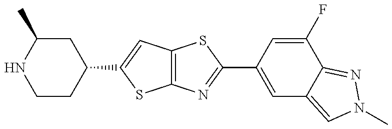 [SMILES: C[C@H]1C[C@H](c2cc3sc(-c4cc(F)c5nn(C)cc5c4)nc3s2)CCN1] |

In some embodiments, for Formula (III), A is monocyclic heterocyclyl (e.g., piperidinyl); B is bicyclic heteroaryl (e.g., 7-fluoro-2-methyl-2H-indazolyl); L¹ and L² are each absent; and R² is hydrogen. In some embodiments, the compound of Formula (III), (III-a), and (III-c) is Compound 106, or a pharmaceutically acceptable salt, solvate, hydrate, tautomer, or stereoisomer thereof.

In some embodiments, for Formula (III), A is monocyclic heterocyclyl (e.g., piperidinyl); B is bicyclic heteroaryl (e.g., 2,8-dimethylimidazo[1,2-b]pyridazinyl); L¹ and L² are each absent; and R² is hydrogen. In some embodiments, the compound of Formula (III), (III-a), and (III-c) is Compound 112, or a pharmaceutically acceptable salt, solvate, hydrate, tautomer, or stereoisomer thereof.

In some embodiments, for Formula (III), A is monocyclic heterocyclyl (e.g., 2,2,6,6-tetramethylpiperidinyl); B is monocyclic heteroaryl (e.g., pyrazolyl); L¹ is —N(R³)— (e.g., —N(CH₃)—); L² is C₆-C₁₂ arylene substituted with one R⁴; R² is hydrogen; and R⁴ is —OR^{A} (e.g., —OH). In some embodiments, the compound of Formula (III) and (III-b) is Compound 118, or a pharmaceutically acceptable salt, solvate, hydrate, tautomer, or stereoisomer thereof.

In some embodiments, for Formula (III), A is bicyclic heteroaryl (e.g., 7-fluoro-2-methyl-2H-indazolyl); B is monocyclic heterocyclyl (e.g., piperidinyl); L¹ and L² are each absent; and R² is hydrogen. In some embodiments, the compound of Formula (III), (III-a), and (III-c) is Compound 124, or a pharmaceutically acceptable salt, solvate, hydrate, tautomer, or stereoisomer thereof.

In some embodiments, for Formula (III), A is bicyclic heteroaryl (e.g., 2,8-dimethylimidazo[1,2-b]pyridazinyl); B is monocyclic heterocyclyl (e.g., piperidinyl); L¹ and L² are each absent; and R² is hydrogen. In some embodiments, the compound of Formula (III), (III-a), and (III-c) is Compound 125, or a pharmaceutically acceptable salt, solvate, hydrate, tautomer, or stereoisomer thereof.

In some embodiments, for Formula (III), A is monocyclic heterocyclyl (e.g., piperidinyl); B is bicyclic heteroaryl (e.g., 4-fluoro-2-methylbenzo[d]thiazolyl); L¹ and L² are each absent; and R² is hydrogen. In some embodiments, the compound of Formula (III), (III-a), and (III-c) is Compound 126, or a pharmaceutically acceptable salt, solvate, hydrate, tautomer, or stereoisomer thereof.

In some embodiments, for Formula (III), A is monocyclic heterocyclyl (e.g., piperidinyl); B is bicyclic heteroaryl (e.g., 2-methyl-2H-indazolyl); L¹ and L² are each absent; and R² is hydrogen. In some embodiments, the compound of Formula (III), (III-a), and (III-c) is Compound 127, or a pharmaceutically acceptable salt, solvate, hydrate, tautomer, or stereoisomer thereof.

In some embodiments, for Formula (III), A is monocyclic heterocyclyl (e.g., 1,2-dimethylpiperazyl); B is bicyclic heteroaryl (e.g., 7-fluoro-2-methyl-2H-indazolyl); L¹ and L² are each absent; and R² is hydrogen. In some embodiments, the compound of Formula (III), (III-a), and (III-c) is Compound 128, or a pharmaceutically acceptable salt, solvate, hydrate, tautomer, or stereoisomer thereof.

In some embodiments, for Formula (III), A is monocyclic heterocyclyl (e.g., 2,2,6,6-tetramethylpiperidinyl); B is bicyclic heteroaryl (e.g., 7-fluoro-2-methyl-2H-indazolyl); L¹ is —N(R³)— (e.g., —N(CH₃)—); L² is absent; and R² is hydrogen. In some embodiments, the compound of Formula (III) is Compound 129, or a pharmaceutically acceptable salt, solvate, hydrate, tautomer, or stereoisomer thereof.

In some embodiments, for Formula (III), A is monocyclic heterocyclyl (e.g., piperidinyl); B is bicyclic heteroaryl (e.g., 8-fluoro-2-methylimidazo[1,2-a]pyridinyl); L¹ and L² are each absent; and R² is hydrogen. In some embodiments, the compound of Formula (III), (III-a), and (III-c) is Compound 130, or a pharmaceutically acceptable salt, solvate, hydrate, tautomer, or stereoisomer thereof.

In some embodiments, for Formula (III), A is monocyclic heterocyclyl (e.g., piperidinyl); B is bicyclic heteroaryl (e.g., 4-fluoro-2-methyl-2H-indazolyl); L¹ and L² are each absent; and R² is hydrogen. In some embodiments, the compound of Formula (III), (III-a), and (III-c) is Compound 131, or a pharmaceutically acceptable salt, solvate, hydrate, tautomer, or stereoisomer thereof.

In some embodiments, for Formula (III), A is monocyclic heterocyclyl (e.g., piperidinyl); B is bicyclic heteroaryl (e.g., 4-fluoro-2-methylbenzo[d]oxazolyl); L¹ and L² are each absent; and R² is hydrogen. In some embodiments, the compound of Formula (III), (III-a), and (III-c) is Compound 132, or a pharmaceutically acceptable salt, solvate, hydrate, tautomer, or stereoisomer thereof.

In some embodiments, for Formula (III), A is monocyclic heterocyclyl (e.g., piperidinyl); B is bicyclic heteroaryl (e.g., 4,6-dimethylpyrazolo[1,5-a]pyrazyl); L¹ and L² are each absent; and R² is hydrogen. In some embodiments, the compound of Formula (III), (III-a), and (III-c) is Compound 133, or a pharmaceutically acceptable salt, solvate, hydrate, tautomer, or stereoisomer thereof.

In some embodiments, for Formula (III), A is monocyclic heterocyclyl (e.g., piperazyl); B is bicyclic heteroaryl (e.g., 7-fluoro-2-methyl-2H-indazolyl); L¹ and L² are each absent; and R² is hydrogen. In some embodiments, the compound of Formula (III), (III-a), and (III-c) is Compound 134, or a pharmaceutically acceptable salt, solvate, hydrate, tautomer, or stereoisomer thereof.

In some embodiments, for Formula (III), A is monocyclic heterocyclyl (e.g., 2-methylpiperazyl); B is bicyclic heteroaryl (e.g., 7-fluoro-2-methyl-2H-indazolyl); L¹ and L² are each absent; and R² is hydrogen. In some embodiments, the compound of Formula (III), (III-a), and (III-c) is Compound 135, or a pharmaceutically acceptable salt, solvate, hydrate, tautomer, or stereoisomer thereof.

In some embodiments, for Formula (III), A is monocyclic heterocyclyl (e.g., 4,7-diazaspiro[2.5]octanyl); B is bicyclic heteroaryl (e.g., 7-fluoro-2-methyl-2H-indazolyl); L¹ and L² are each absent; and R² is hydrogen. In some embodiments, the compound of Formula (III), (III-a), and (III-c) is Compound 136, or a pharmaceutically acceptable salt, solvate, hydrate, tautomer, or stereoisomer thereof.

In some embodiments, for Formula (III), A is monocyclic heterocyclyl (e.g., piperidinyl) substituted with one R¹; B is bicyclic heteroaryl (e.g., 7-fluoro-2-methyl-2H-indazolyl); L¹ and L² are each absent; R¹ is —NR^{B}R^{C} (e.g., —NH(CH₂CH₃)); and R² is hydrogen. In some embodiments, the compound of Formula (III), (III-a), and (III-c) is Compound 137, or a pharmaceutically acceptable salt, solvate, hydrate, tautomer, or stereoisomer thereof.

In some embodiments, for Formula (III), A is monocyclic heterocyclyl (e.g., piperidinyl); B is bicyclic heteroaryl (e.g., 2,7-dimethylimidazo[1,2-a]pyridinyl); L¹ and L² are each absent; and R² is hydrogen. In some embodiments, the compound of Formula (III), (III-a), and (III-c) is Compound 138, or a pharmaceutically acceptable salt, solvate, hydrate, tautomer, or stereoisomer thereof.

In some embodiments, for Formula (III), A is monocyclic heterocyclyl (e.g., piperidinyl); B is bicyclic heteroaryl (e.g., 8-chloro-2-methylimidazo[1,2-a]pyridinyl); L¹ and L² are each absent; and R² is hydrogen. In some embodiments, the compound of Formula (III), (III-a), and (III-c) is Compound 139, or a pharmaceutically acceptable salt, solvate, hydrate, tautomer, or stereoisomer thereof.

In some embodiments, for Formula (III), A is monocyclic heterocyclyl (e.g., piperidinyl); B is bicyclic heteroaryl (e.g., 2,8-dimethylimidazo[1,2-a]pyridinyl); L¹ and L² are each absent; and R² is hydrogen. In some embodiments, the compound of Formula (III), (III-a), and (III-c) is Compound 140, or a pharmaceutically acceptable salt, solvate, hydrate, tautomer, or stereoisomer thereof.

In some embodiments, for Formula (III), A is monocyclic heterocyclyl (e.g., 1-methylpiperazyl); B is bicyclic heteroaryl (e.g., 7-fluoro-2-methyl-2H-indazolyl); L¹ and L² are each absent; and R² is hydrogen. In some embodiments, the compound of Formula (III), (III-a), and (III-c) is Compound 141, or a pharmaceutically acceptable salt, solvate, hydrate, tautomer, or stereoisomer thereof.

In some embodiments, for Formula (III), A is monocyclic heterocyclyl (e.g., 2,2-dimethylpiperazyl); B is bicyclic heteroaryl (e.g., 7-fluoro-2-methyl-2H-indazolyl); L¹ and L² are each absent; and R² is hydrogen. In some embodiments, the compound of Formula (III), (III-a), and (III-c) is Compound 142, or a pharmaceutically acceptable salt, solvate, hydrate, tautomer, or stereoisomer thereof.

In some embodiments, for Formula (III), A is monocyclic heterocyclyl (e.g., pyrrolidinyl) substituted with one R¹; B is bicyclic heteroaryl (e.g., 7-fluoro-2-methyl-2H-indazolyl); L¹ and L² are each absent; R¹ is —NR^{B}R^{C} (e.g., —NH(^{t}Bu)); and R² is hydrogen. In some embodiments, the compound of Formula (III), (III-a), and (III-c) is Compound 143, or a pharmaceutically acceptable salt, solvate, hydrate, tautomer, or stereoisomer thereof.

In some embodiments, for Formula (III), A is monocyclic heterocyclyl (e.g., hexahydro-1H-pyrrolo[2,1-c]pyrazyl); B is bicyclic heteroaryl (e.g., 7-fluoro-2-methyl-2H-indazolyl); L¹ and L² are each absent; and R² is hydrogen. In some embodiments, the compound of Formula (III), (III-a), and (III-c) is Compound 144, or a pharmaceutically acceptable salt, solvate, hydrate, tautomer, or stereoisomer thereof.

In some embodiments, for Formula (III), A is monocyclic heterocyclyl (e.g., 1-methylpiperazyl); B is bicyclic heteroaryl (e.g., 7-fluoro-2-methyl-2H-indazolyl); L¹ is —N(R³)— (e.g., —N(CH₃)—); L² is absent; and R² is hydrogen. In some embodiments, the compound of Formula (III) is Compound 145, or a pharmaceutically acceptable salt, solvate, hydrate, tautomer, or stereoisomer thereof.

In some embodiments, for Formula (III), A is bicyclic heterocyclyl (e.g 2-methyl-2,6-diazaspiro[3.3]heptanyl); B is bicyclic heteroaryl (e.g., 7-fluoro-2-methyl-2H-indazolyl); L¹ and L² are each absent; and R² is hydrogen. In some embodiments, the compound of Formula (III), (III-a), and (III-c) is Compound 146, or a pharmaceutically acceptable salt, solvate, hydrate, tautomer, or stereoisomer thereof.

In some embodiments, for Formula (III), A is monocyclic heterocyclyl (e.g piperazyl); B is bicyclic heteroaryl (e.g., 7-fluoro-2-methyl-2H-indazolyl); L¹ and L² are each absent; and R² is halo (e.g., —Cl). In some embodiments, the compound of Formula (III) and (III-c) is Compound 147, or a pharmaceutically acceptable salt, solvate, hydrate, tautomer, or stereoisomer thereof.

In some embodiments, for Formula (III), A is monocyclic heterocyclyl (e.g., 1-methylpiperidinyl); B is bicyclic heteroaryl (e.g., 7-fluoro-2-methyl-2H-indazolyl); L¹ and L² are each absent; and R² is hydrogen. In some embodiments, the compound of Formula (III), (III-a), and (III-c) is Compound 151, or a pharmaceutically acceptable salt, solvate, hydrate, tautomer, or stereoisomer thereof.

In some embodiments, for Formula (III), A is monocyclic heterocyclyl (e.g., piperidinyl); B is monocyclic heteroaryl (e.g., pyrazolyl); L¹ and L² are each absent; and R² is hydrogen. In some embodiments, the compound of Formula (III), (III-a), and (III-c) is Compound 152, or a pharmaceutically acceptable salt, solvate, hydrate, tautomer, or stereoisomer thereof.

In some embodiments, for Formula (III), A is monocyclic heterocyclyl (e.g piperidinyl) substituted with one R¹; B is bicyclic heteroaryl (e.g., 7-fluoro-2-methyl-2H-indazolyl); L¹ and L² are each absent; R¹ is —NR^{B}R^{C} (e.g., —NH(CH₂CH₃)); and R² is halo (e.g., —Cl). In some embodiments, the compound of Formula (III) and (III-c) is Compound 153, or a pharmaceutically acceptable salt, solvate, hydrate, tautomer, or stereoisomer thereof.

In some embodiments, for Formula (III), A is monocyclic heterocyclyl (e.g., piperidinyl) substituted with one R¹; B is bicyclic heteroaryl (e.g., 7-fluoro-2-methyl-2H-indazolyl); L¹ and L² are each absent; R¹ is —NR^{B}N^{C} (e.g., —N(CH₃)₂); and R² is hydrogen. In some embodiments, the compound of Formula (III), (III-a), and (III-c) is Compound 154, or a pharmaceutically acceptable salt, solvate, hydrate, tautomer, or stereoisomer thereof.

In some embodiments, for Formula (III), A is bicyclic heterocyclyl (e.g., 8-azabicyclo[3.2.1]octanyl); B is monocyclic heteroaryl (e.g., pyrazolyl); L¹ is —N(R³)— (e.g., —N(CH₃)—); L² is C₆-C₁₂ arylene substituted with one R⁴; R² is hydrogen; and R⁴ is —OR^{A} (e.g., —OH). In some embodiments, the compound of Formula (III) and (III-b) is Compound 191, or a pharmaceutically acceptable salt, solvate, hydrate, tautomer, or stereoisomer thereof.

In some embodiments, for Formula (III), A is monocyclic heterocyclyl (e.g., 2-methylpiperidinyl); B is bicyclic heteroaryl (e.g., 7-fluoro-2-methyl-2H-indazolyl); L¹ and L² are each absent; and R² is hydrogen. In some embodiments, the compound of Formula (III), (III-a), and (III-c) is Compound 192, 193, or a pharmaceutically acceptable salt, solvate, hydrate, tautomer, or stereoisomer thereof.

In some embodiments, for Formula (III), A is monocyclic heterocyclyl (e.g., piperazyl); B is bicyclic heteroaryl (e.g., 7-fluoro-2-methyl-2H-indazolyl); L¹ and L² are each absent; and R² is C₁-C₆ alkyl (CH₃). In some embodiments, the compound of Formula (III) and (III-c) is Compound 194, or a pharmaceutically acceptable salt, solvate, hydrate, tautomer, or stereoisomer thereof.

In some embodiments, for Formula (III), A is monocyclic heterocyclyl (e.g., 3,6-dihydro-2H-pyranyl); B is bicyclic heteroaryl (e.g., 2,8-dimethylimidazo[1,2-b]pyridazyl); L¹ and L² are each absent; and R² is hydrogen. In some embodiments, the compound of Formula (III), (III-a), and (III-c) is Compound 195, or a pharmaceutically acceptable salt, solvate, hydrate, tautomer, or stereoisomer thereof.

In some embodiments, for Formula (III), A is monocyclic heterocyclyl (e.g., tetrahydropyranyl); B is bicyclic heteroaryl (e.g., 2,8-dimethylimidazo[1,2-b]pyridazyl); L¹ and L² are each absent; and R² is hydrogen. In some embodiments, the compound of Formula (III), (III-a), and (III-c) is Compound 196, or a pharmaceutically acceptable salt, solvate, hydrate, tautomer, or stereoisomer thereof.

In some embodiments, for Formula (III), A is monocyclic heterocyclyl (e.g., pyrrolidinyl); B is bicyclic heteroaryl (e.g., 2,8-dimethylimidazo[1,2-b]pyridazyl); L¹ and L² are each absent; and R² is hydrogen. In some embodiments, the compound of Formula (III), (III-a), and (III-c) is Compound 197, or a pharmaceutically acceptable salt, solvate, hydrate, tautomer, or stereoisomer thereof.

In some embodiments, for Formula (III), A is monocyclic heterocyclyl (e.g., 1,2,3,6-tetrahydropyridinyl); B is bicyclic heteroaryl (e.g., 2,8-dimethylimidazo[1,2-b]pyridazyl); L¹ and L² are each absent; and R² is hydrogen. In some embodiments, the compound of Formula (III), (III-a), and (III-c) is Compound 198, or a pharmaceutically acceptable salt, solvate, hydrate, tautomer, or stereoisomer thereof.

In some embodiments, for Formula (III), A is monocyclic heterocyclyl (e.g., piperidinyl); B is bicyclic heteroaryl (e.g., 2-methyl-6-hydroxy-2H-indazolyl); L¹ and L² are each absent; and R² is hydrogen. In some embodiments, the compound of Formula (III), (III-a), and (III-c) is Compound 199, or a pharmaceutically acceptable salt, solvate, hydrate, tautomer, or stereoisomer thereof.

In some embodiments, for Formula (III), A is bicyclic heterocyclyl (e.g., 1,6-diazaspiro[3.4]octanyl); B is bicyclic heteroaryl (e.g., 7-fluoro-2-methyl-2H-indazolyl); L¹ and L² are each absent; and R² is hydrogen. In some embodiments, the compound of Formula (III), (III-a), and (III-c) is Compound 200, or a pharmaceutically acceptable salt, solvate, hydrate, tautomer, or stereoisomer thereof.

In some embodiments, for Formula (III), A is bicyclic heterocyclyl (e.g., 1,7-diazaspiro[3.5]nonanyl); B is bicyclic heteroaryl (e.g., 7-fluoro-2-methyl-2H-indazolyl); L¹ and L² are each absent; and R² is hydrogen. In some embodiments, the compound of Formula (III), (III-a), and (III-c) is Compound 201, or a pharmaceutically acceptable salt, solvate, hydrate, tautomer, or stereoisomer thereof.

In some embodiments, for Formula (III), A is bicyclic heterocyclyl (e.g., 1,6-diazaspiro[3.5]nonanyl); B is bicyclic heteroaryl (e.g., 7-fluoro-2-methyl-2H-indazolyl); L¹ and L² are each absent; and R² is hydrogen. In some embodiments, the compound of Formula (III), (III-a), and (III-c) is Compound 202, or a pharmaceutically acceptable salt, solvate, hydrate, tautomer, or stereoisomer thereof.

In some embodiments, for Formula (III), A is bicyclic heterocyclyl (e.g., 6-methyl-1,6-diazaspiro[3.5]nonanyl); B is bicyclic heteroaryl (e.g., 7-fluoro-2-methyl-2H-indazolyl); L¹ and L² are each absent; and R² is hydrogen. In some embodiments, the compound of Formula (III), (III-a), and (III-c) is Compound 203, or a pharmaceutically acceptable salt, solvate, hydrate, tautomer, or stereoisomer thereof.

In some embodiments, for Formula (III), A is bicyclic heterocyclyl (e.g., 7-methyl-1,7-diazaspiro[3.5]nonanyl); B is bicyclic heteroaryl (e.g., 7-fluoro-2-methyl-2H-indazolyl); L¹ and L² are each absent; and R² is hydrogen. In some embodiments, the compound of Formula (III), (III-a), and (III-c) is Compound 204, or a pharmaceutically acceptable salt, solvate, hydrate, tautomer, or stereoisomer thereof.

In some embodiments, for Formula (III), A is monocyclic heterocyclyl (e.g., piperidinyl) substituted with one R¹; B is bicyclic heteroaryl (e.g., 7-fluoro-2-methyl-2H-indazolyl); L¹ and L² are each absent; R¹ is C₁-C₆ alkyl (e.g., ethyl); and R² is hydrogen. In some embodiments, the compound of Formula (III), (III-a), and (III-c) is Compound 230, or a pharmaceutically acceptable salt, solvate, hydrate, tautomer, or stereoisomer thereof.

In some embodiments, for Formula (III), A is monocyclic heterocyclyl (e.g., piperidinyl); B is bicyclic heteroaryl (e.g., 2-methyl-4-hydroxy-2H-indazolyl); L¹ and L² are each absent; and R² is hydrogen. In some embodiments, the compound of Formula (III), (III-a), and (III-c) is Compound 231, or a pharmaceutically acceptable salt, solvate, hydrate, tautomer, or stereoisomer thereof.

In some embodiments, for Formula (III), A is monocyclic heterocyclyl (e.g., piperidinyl); B is bicyclic heteroaryl (e.g., 3-hydroxy-4,6-dimethylpyrazolo[1,5-a]pyrazyl); L¹ and L² are each absent; and R² is hydrogen. In some embodiments, the compound of Formula (III), (III-a), and (III-c) is Compound 232, or a pharmaceutically acceptable salt, solvate, hydrate, tautomer, or stereoisomer thereof.

In some embodiments, for Formula (III), A is bicyclic heterocyclyl (e.g., 1,6-diazaspiro[3.4]octanyl); B is bicyclic heteroaryl (e.g., 7-fluoro-2-methyl-2H-indazolyl); L¹ and L² are each absent; and R² is hydrogen. In some embodiments, the compound of Formula (III), (III-a), and (III-c) is Compound 233, or a pharmaceutically acceptable salt, solvate, hydrate, tautomer, or stereoisomer thereof.

In some embodiments, for Formula (III), A is bicyclic heterocyclyl (e.g., 1,6-diazaspiro[3.4]octanyl) substituted with one R¹; B is bicyclic heteroaryl (e.g., 7-fluoro-2-methyl-2H-indazolyl); L¹ and L² are each absent; R¹ is C₁-C₆ alkyl (e.g., —CH₃); and R² is hydrogen. In some embodiments, the compound of Formula (III), (III-a), and (III-c) is Compound 234, or a pharmaceutically acceptable salt, solvate, hydrate, tautomer, or stereoisomer thereof.

In some embodiments, for Formula (III), A is monocyclic heterocyclyl (e.g., azetidinyl); B is bicyclic heteroaryl (e.g., 2,8-dimethylimidazo[1,2-b]pyridazyl); L¹ and L² are each absent; and R² is hydrogen. In some embodiments, the compound of Formula (III), (III-a), and (III-c) is Compound 235, or a pharmaceutically acceptable salt, solvate, hydrate, tautomer, or stereoisomer thereof.

In some embodiments, for Formula (III), A is bicyclic heterocyclyl (e.g., piperidinyl) substituted with one R¹; B is bicyclic heteroaryl (e.g., 7-fluoro-2-methyl-2H-indazolyl); L¹ and L² are each absent; R¹ is —OR^{A} (e.g., —OH); and R² is hydrogen. In some embodiments, the compound of Formula (III), (III-a), and (III-c) is Compound 236, or a pharmaceutically acceptable salt, solvate, hydrate, tautomer, or stereoisomer thereof.

In some embodiments, for Formula (III), A is bicyclic heterocyclyl (e.g., piperidinyl); B is bicyclic heteroaryl (e.g., 7-fluoro-4-methoxy-2-methyl-2H-indazolyl); L¹ and L² are each absent; and R² is hydrogen. In some embodiments, the compound of Formula (III), (III-a), and (III-c) is Compound 237, or a pharmaceutically acceptable salt, solvate, hydrate, tautomer, or stereoisomer thereof.

In some embodiments, for Formula (III), A is bicyclic heterocyclyl (e.g., piperidinyl); B is bicyclic heteroaryl (e.g., 2,7-dimethyl-2H-indazolyl); L¹ and L² are each absent; and R² is hydrogen. In some embodiments, the compound of Formula (III), (111-a), and (111-c) is Compound 238, or a pharmaceutically acceptable salt, solvate, hydrate, tautomer, or stereoisomer thereof.

In some embodiments, for Formula (III), A is bicyclic heterocyclyl (e.g., piperidinyl); B is bicyclic heteroaryl (e.g., 6,8-dimethylimidazo[1,2-a]pyrazyl); L^{r} and L² are each absent; and R² is hydrogen. In some embodiments, the compound of Formula (III), (III-a), and (III-c) is Compound 239, or a pharmaceutically acceptable salt, solvate, hydrate, tautomer, or stereoisomer thereof.

In some embodiments, for Formula (III), A is bicyclic heterocyclyl (e.g., piperidinyl) substituted with one R¹; B is bicyclic heteroaryl (e.g., 7-fluoro-2-methyl-2H-indazolyl); L¹ and L² are each absent; R^{r} is halo (e.g., —F); and R² is hydrogen. In some embodiments, the compound of Formula (III), (III-a), and (III-c) is Compound 240, or a pharmaceutically acceptable salt, solvate, hydrate, tautomer, or stereoisomer thereof.

In some embodiments, for Formula (III), A is bicyclic heterocyclyl (e.g., piperidinyl) substituted with two R¹; B is bicyclic heteroaryl (e.g., 7-fluoro-2-methyl-2H-indazolyl); L¹ and L² are each absent; each R^{r} is independently halo (e.g., —F); and R² is hydrogen. In some embodiments, the compound of Formula (III), (III-a), and (III-c) is Compound 241, or a pharmaceutically acceptable salt, solvate, hydrate, tautomer, or stereoisomer thereof.

In some embodiments, the present disclosure features a compound of Formula (IV-a):

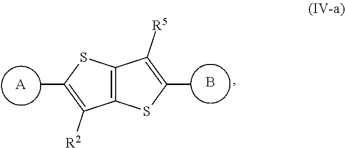

or a pharmaceutically acceptable salt, solvate, hydrate, tautomer, or stereoisomer thereof, wherein A and B are each independently cycloalkyl, heterocyclyl, aryl, or heteroaryl, each of which is optionally substituted with one or more R¹; each R¹ is independently hydrogen, C₁-C₆-alkyl, C₂-C₆-alkenyl, C₂-C₆-alkynyl, C₁-C₆-heteroalkyl, C₁-C₆-haloalkyl, cycloalkyl, heterocyclyl, aryl, C₁-C₆ alkylene-aryl, C₁-C₆ alkenylene-aryl, C₁-C₆ alkylene-heteroaryl, heteroaryl, halo, cyano, oxo, —OR^{A}, —NR^{B}R^{C}, —NR^{B}C(O)R^{D}, —NO₂, —C(O)NR^{B}R^{C}, —C(O)R^{D}, —C(O)OR^{D}, or —S(O)ₓR^{D}, wherein each alkyl, alkylene, alkenyl, alkynyl, heteroalkyl, haloalkyl, cycloalkyl, heterocyclyl, aryl, and heteroaryl is optionally substituted with one or more R⁶; or two R¹ groups, together with the atoms to which they are attached, form a 3-7-membered cycloalkyl, heterocyclyl, aryl, or heteroaryl, wherein each cycloalkyl, heterocyclyl, aryl, and heteroaryl is optionally substituted with one or more R⁶; R² and R⁵ are each independently is hydrogen, halo, cyano, C₁-C₆-alkyl, C₂-C₆-alkenyl, C₂-C₆-alkynyl, C₁-C₆-heteroalkyl, or C₁-C₆-haloalkyl; each R⁶ is independently C₁-C₆-alkyl, C₂-C₆-alkenyl, C₂-C₆-alkynyl, C₁-C₆-heteroalkyl, C₁-C₆-haloalkyl, cycloalkyl, heterocyclyl, aryl, heteroaryl, halo, cyano, oxo, —OR^{A}, —NR^{B}R^{C}, NR^{B}C(O)R^{D}, —NO₂, —C(O)NR^{B}R^{C}, —C(O)R^{D}, —C(O)OR^{D}, or —S(O)ₓR^{D}, wherein each of alkyl, alkenyl, alkynyl, heteroalkyl, haloalkyl, cycloalkyl, heterocyclyl, aryl, and heteroaryl is optionally substituted with one or more R⁷; each R^{A} is independently hydrogen, C₁-C₆ alkyl, C₁-C₆ haloalkyl, aryl, heteroaryl, C₁-C₆ alkylene-aryl, C₁-C₆ alkylene-heteroaryl, —C(O)R^{D}, or —S(O)ₓR^{D}; each of R^{B} and R^{C} is independently hydrogen, C₁-C₆ alkyl, C₁-C₆ heteroalkyl, cycloalkyl, heterocyclyl, or —OR^{A}; or R^{B} and R^{C} together with the atom to which they are attached form a 3-7-membered heterocyclyl ring optionally substituted with one or more R⁸; each R^{D} is independently hydrogen, C₁-C₆ alkyl, C₂-C₆ alkenyl, C₂-C₆ alkynyl, C₁-C₆ heteroalkyl, C₁-C₆ haloalkyl, cycloalkyl, heterocyclyl, aryl, heteroaryl, C₁-C₆ alkylene-aryl, or C₁-C₆ alkylene-heteroaryl; each R⁷ is independently C₁-C₆ alkyl, C₁-C₆ heteroalkyl, C₁-C₆ haloalkyl, cycloalkyl, heterocyclyl, aryl, heteroaryl, halo, cyano, oxo, or —OR^{A}; each R⁸ is C₁-C₆-alkyl, halo, cyano, oxo, or —OR^{A1}; each R^{A1} is hydrogen or C₁-C₆-alkyl; and x is 0, 1, or 2.

In some embodiments, the present disclosure features a compound of Formula (IV-b):

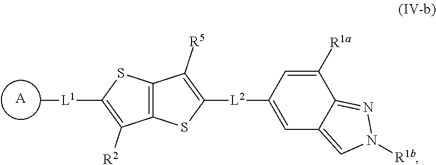

or a pharmaceutically acceptable salt, solvate, hydrate, tautomer, or stereoisomer thereof, wherein A is cycloalkyl, heterocyclyl, aryl, or heteroaryl, each of which is optionally substituted with one or more R¹; L¹ is absent, C₁-C₆-alkylene, C₁-C₆-heteroalkylene, —O—, —C(O)—, —N(R³)—, —N(R³)C(O)—, or —C(O)N(R³)—, wherein each alkylene and heteroalkylene is optionally substituted with one or more R⁴; L² is absent, C₁-C₆-alkylene, C₁-C₆-heteroalkylene, C₆-C₁₂-arylene, C₅-C₁₂-heteroarylene, —O—, —C(O)—, —N(R³)—, —N(R³)C(O)—, or —C(O)N(R³)—, wherein each alkylene, heteroalkylene, arylene, and heteroarylene is optionally substituted with one or more R⁴; each R¹, R^{1a}, and R^{1b} is independently hydrogen, C₁-C₆-alkyl, C₂-C₆-alkenyl, C₂-C₆-alkynyl, C₁-C₆-heteroalkyl, C₁-C₆-haloalkyl, cycloalkyl, heterocyclyl, aryl, C₁-C₆ alkylene-aryl, C₁-C₆ alkenylene-aryl, C₁-C₆ alkylene-heteroaryl, heteroaryl, halo, cyano, oxo, —OR^{A}, —NR^{B}R^{C}, —N^{B}C(O)R^{D}, —NO₂, —C(O)NR^{B}R^{C}, —C(O)R^{D}, —C(O)OR^{D}, or —S(O)ₓR^{D}, wherein each alkyl, alkylene, alkenyl, alkynyl, heteroalkyl, haloalkyl, cycloalkyl, heterocyclyl, aryl, and heteroaryl is optionally substituted with one or more R⁶; or two R¹ groups, together with the atoms to which they are attached, form a 3-7-membered cycloalkyl, heterocyclyl, aryl, or heteroaryl, wherein each cycloalkyl, heterocyclyl, aryl, and heteroaryl is optionally substituted with one or more R⁶; R² and R⁵ are each independently is hydrogen, halo, cyano, C₁-C₆-alkyl, C₂-C₆-alkenyl, C₂-C₆-alkynyl, C₁-C₆-heteroalkyl, or C₁-C₆-haloalkyl; each R⁶ is independently C₁-C₆-alkyl, C₂-C₆-alkenyl, C₂-C₆-alkynyl, C₁-C₆-heteroalkyl, C₁-C₆-haloalkyl, cycloalkyl, heterocyclyl, aryl, heteroaryl, halo, cyano, oxo, —OR^{A}, —NR^{B}R^{C}, —N^{B}C(O)R^{D}, —NO₂, —C(O)NR^{B}R^{C}, —C(O)R^{D}, —C(O)OR^{D}, or —S(O)ₓR^{D}, wherein each of alkyl, alkenyl, alkynyl, heteroalkyl, haloalkyl, cycloalkyl, heterocyclyl, aryl, and heteroaryl is optionally substituted with one or more R⁷; each R^{A} is independently hydrogen, C₁-C₆ alkyl, C₁-C₆ haloalkyl, aryl, heteroaryl, C₁-C₆ alkylene-aryl, C₁-C₆ alkylene-heteroaryl, —C(O)R^{D}, or —S(O)ₓR^{D}; each of R^{B} and R^{C} is independently hydrogen, C₁-C₆ alkyl, C₁-C₆ heteroalkyl, cycloalkyl, heterocyclyl, or —OR^{A}; or R^{B} and R^{C} together with the atom to which they are attached form a 3-7-membered heterocyclyl ring optionally substituted with one or more R⁸; each R^{D} is independently hydrogen, C₁-C₆ alkyl, C₂-C₆ alkenyl, C₂-C₆ alkynyl, C₁-C₆ heteroalkyl, C₁-C₆ haloalkyl, cycloalkyl, heterocyclyl, aryl, heteroaryl, C₁-C₆ alkylene-aryl, or C₁-C₆ alkylene-heteroaryl; each R⁷ is independently C₁-C₆ alkyl, C₁-C₆ heteroalkyl, C₁-C₆ haloalkyl, cycloalkyl, heterocyclyl, aryl, heteroaryl, halo, cyano, oxo, or —OR^{A}; each R⁸ is C₁-C₆-alkyl, halo, cyano, oxo, or —OR^{A1}; each R^{A1} is hydrogen or C₁-C₆-alkyl; and x is 0, 1, or 2.

In some embodiments, the compound of Formula (IV) is selected from a compound in Table 4, or a pharmaceutically acceptable salt, solvate, hydrate, tautomer, or stereoisomer thereof.

TABLE 4
Exemplary compounds of Formula (IV)

| Compound | |
|---|---|
| No. | Structure |
| 208 | 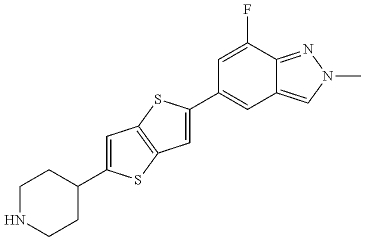 [SMILES: Cn1cc2cc(-c3cc4sc(C5CCNCC5)cc4s3)cc(F)c2n1] |
| 209 | 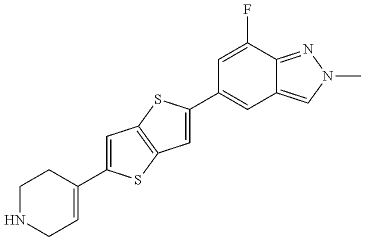 [SMILES: Cn1cc2cc(-c3cc4sc(C5=CCNCC5)cc4s3)cc(F)c2n1] |

### Pharmaceutical Compositions, Kits, and Administration

The present invention provides pharmaceutical compositions comprising a compound of Formula (I), (II), (III), or (IV), e.g., a compound of Formula (I), (II), (III), or (IV), or a pharmaceutically acceptable salt, solvate, hydrate, tautomer, or stereoisomer, as described herein, and optionally a pharmaceutically acceptable excipient. In certain embodiments, the pharmaceutical composition described herein comprises a compound of Formula (I), (II), (III), or (IV), or a pharmaceutically acceptable salt thereof, and optionally a pharmaceutically acceptable excipient. In certain embodiments, the compound of Formula (I), (II), (III), or (IV), or a pharmaceutically acceptable salt, solvate, hydrate, tautomer, or stereoisomer thereof, is provided in an effective amount in the pharmaceutical composition. In certain embodiments, the effective amount is a therapeutically effective amount. In certain embodiments, the effective amount is a prophylactically effective amount.

Pharmaceutical compositions described herein can be prepared by any method known in the art of pharmacology. In general, such preparatory methods include the steps of bringing the compound of Formula (I), (II), (III), or (IV) (the “active ingredient”) into association with a carrier and/or one or more other accessory ingredients, and then, if necessary and/or desirable, shaping and/or packaging the product into a desired single- or multi-dose unit.

Pharmaceutical compositions can be prepared, packaged, and/or sold in bulk, as a single unit dose, and/or as a plurality of single unit doses. As used herein, a “unit dose” is a discrete amount of the pharmaceutical composition comprising a predetermined amount of the active ingredient. The amount of the active ingredient is generally equal to the dosage of the active ingredient which would be administered to a subject and/or a convenient fraction of such a dosage such as, for example, one-half or one-third of such a dosage.

Relative amounts of the active ingredient, the pharmaceutically acceptable excipient, and/or any additional ingredients in a pharmaceutical composition of the invention will vary, depending upon the identity, size, and/or condition of the subject treated and further depending upon the route by which the composition is to be administered. By way of example, the composition may comprise between 0.1% and 100% (w/w) active ingredient.

The term “pharmaceutically acceptable excipient” refers to a non-toxic carrier, adjuvant, diluent, or vehicle that does not destroy the pharmacological activity of the compound with which it is formulated. Pharmaceutically acceptable excipients useful in the manufacture of the pharmaceutical compositions of the invention are any of those that are well known in the art of pharmaceutical formulation and include inert diluents, dispersing and/or granulating agents, surface active agents and/or emulsifiers, disintegrating agents, binding agents, preservatives, buffering agents, lubricating agents, and/or oils. Pharmaceutically acceptable excipients useful in the manufacture of the pharmaceutical compositions of the invention include, but are not limited to, ion exchangers, alumina, aluminum stearate, lecithin, serum proteins, such as human serum albumin, buffer substances such as phosphates, glycine, sorbic acid, potassium sorbate, partial glyceride mixtures of saturated vegetable fatty acids, water, salts or electrolytes, such as protamine sulfate, disodium hydrogen phosphate, potassium hydrogen phosphate, sodium chloride, zinc salts, colloidal silica, magnesium trisilicate, polyvinyl pyrrolidone, cellulose-based substances, polyethylene glycol, sodium carboxymethylcellulose, polyacrylates, waxes, polyethylene-polyoxypropylene-block polymers, polyethylene glycol and wool fat.

Compositions of the present invention may be administered orally, parenterally (including subcutaneous, intramuscular, intravenous and intradermal), by inhalation spray, topically, rectally, nasally, buccally, vaginally or via an implanted reservoir. In some embodiments, provided compounds or compositions are administrable intravenously and/or orally.

The term “parenteral” as used herein includes subcutaneous, intravenous, intramuscular, intraocular, intravitreal, intra-articular, intra-synovial, intrasternal, intrathecal, intrahepatic, intraperitoneal intralesional and intracranial injection or infusion techniques. Preferably, the compositions are administered orally, subcutaneously, intraperitoneally, or intravenously. Sterile injectable forms of the compositions of this invention may be aqueous or oleaginous suspension. These suspensions may be formulated according to techniques known in the art using suitable dispersing or wetting agents and suspending agents. The sterile injectable preparation may also be a sterile injectable solution or suspension in a non-toxic parenterally acceptable diluent or solvent, for example as a solution in 1,3-butanediol. Among the acceptable vehicles and solvents that may be employed are water, Ringer's solution and isotonic sodium chloride solution. In addition, sterile, fixed oils are conventionally employed as a solvent or suspending medium.

Pharmaceutically acceptable compositions of this invention may be orally administered in any orally acceptable dosage form including, but not limited to, capsules, tablets, aqueous suspensions or solutions. In the case of tablets for oral use, carriers commonly used include lactose and corn starch. Lubricating agents, such as magnesium stearate, are also typically added. For oral administration in a capsule form, useful diluents include lactose and dried cornstarch. When aqueous suspensions are required for oral use, the active ingredient is combined with emulsifying and suspending agents. If desired, certain sweetening, flavoring or coloring agents may also be added. In some embodiments, a provided oral formulation is formulated for immediate release or sustained/delayed release. In some embodiments, the composition is suitable for buccal or sublingual administration, including tablets, lozenges and pastilles. A provided compound can also be in micro-encapsulated form.

Alternatively, pharmaceutically acceptable compositions of this invention may be administered in the form of suppositories for rectal administration. Pharmaceutically acceptable compositions of this invention may also be administered topically, especially when the target of treatment includes areas or organs readily accessible by topical application, including diseases of the eye, the skin, or the lower intestinal tract. Suitable topical formulations are readily prepared for each of these areas or organs.

For ophthalmic use, provided pharmaceutically acceptable compositions may be formulated as micronized suspensions or in an ointment such as petrolatum.

In order to prolong the effect of a drug, it is often desirable to slow the absorption of the drug from subcutaneous or intramuscular injection. This can be accomplished by the use of a liquid suspension of crystalline or amorphous material with poor water solubility. The rate of absorption of the drug then depends upon its rate of dissolution which, in turn, may depend upon crystal size and crystalline form. Alternatively, delayed absorption of a parenterally administered drug form is accomplished by dissolving or suspending the drug in an oil vehicle.

Although the descriptions of pharmaceutical compositions provided herein are principally directed to pharmaceutical compositions which are suitable for administration to humans, it will be understood by the skilled artisan that such compositions are generally suitable for administration to animals of all sorts. Modification of pharmaceutical compositions suitable for administration to humans in order to render the compositions suitable for administration to various animals is well understood, and the ordinarily skilled veterinary pharmacologist can design and/or perform such modification with ordinary experimentation.

Compounds provided herein are typically formulated in dosage unit form, e.g., single unit dosage form, for ease of administration and uniformity of dosage. It will be understood, however, that the total daily usage of the compositions of the present invention will be decided by the attending physician within the scope of sound medical judgment. The specific therapeutically effective dose level for any particular subject or organism will depend upon a variety of factors including the disease being treated and the severity of the disorder; the activity of the specific active ingredient employed; the specific composition employed; the age, body weight, general health, sex and diet of the subject; the time of administration, route of administration, and rate of excretion of the specific active ingredient employed; the duration of the treatment; drugs used in combination or coincidental with the specific active ingredient employed; and like factors well known in the medical arts.

The exact amount of a compound required to achieve an effective amount will vary from subject to subject, depending, for example, on species, age, and general condition of a subject, severity of the side effects or disorder, identity of the particular compound(s), mode of administration, and the like. The desired dosage can be delivered three times a day, two times a day, once a day, every other day, every third day, every week, every two weeks, every three weeks, or every four weeks. In certain embodiments, the desired dosage can be delivered using multiple administrations (e.g., two, three, four, five, six, seven, eight, nine, ten, eleven, twelve, thirteen, fourteen, or more administrations).

In certain embodiments, an effective amount of a compound for administration one or more times a day to a 70 kg adult human may comprise about 0.0001 mg to about 3000 mg, about 0.0001 mg to about 2000 mg, about 0.0001 mg to about 1000 mg, about 0.001 mg to about 1000 mg, about 0.01 mg to about 1000 mg, about 0.1 mg to about 1000 mg, about 1 mg to about 1000 mg, about 1 mg to about 100 mg, about 10 mg to about 1000 mg, or about 100 mg to about 1000 mg, of a compound per unit dosage form.

In certain embodiments, the compounds of Formula (I), (II), (III), or (IV) may be at dosage levels sufficient to deliver from about 0.001 mg/kg to about 100 mg/kg, from about 0.01 mg/kg to about 50 mg/kg, preferably from about 0.1 mg/kg to about 40 mg/kg, preferably from about 0.5 mg/kg to about 30 mg/kg, from about 0.01 mg/kg to about 10 mg/kg, from about 0.1 mg/kg to about 10 mg/kg, and more preferably from about 1 mg/kg to about 25 mg/kg, of subject body weight per day, one or more times a day, to obtain the desired therapeutic effect.

It will be appreciated that dose ranges as described herein provide guidance for the administration of provided pharmaceutical compositions to an adult. The amount to be administered to, for example, a child or an adolescent can be determined by a medical practitioner or person skilled in the art and can be lower or the same as that administered to an adult.

It will be also appreciated that a compound or composition, as described herein, can be administered in combination with one or more additional pharmaceutical agents. The compounds or compositions can be administered in combination with additional pharmaceutical agents that improve their bioavailability, reduce and/or modify their metabolism, inhibit their excretion, and/or modify their distribution within the body. It will also be appreciated that the therapy employed may achieve a desired effect for the same disorder, and/or it may achieve different effects.

The compound or composition can be administered concurrently with, prior to, or subsequent to, one or more additional pharmaceutical agents, which may be useful as, e.g., combination therapies. Pharmaceutical agents include therapeutically active agents. Pharmaceutical agents also include prophylactically active agents. Each additional pharmaceutical agent may be administered at a dose and/or on a time schedule determined for that pharmaceutical agent. The additional pharmaceutical agents may also be administered together with each other and/or with the compound or composition described herein in a single dose or administered separately in different doses. The particular combination to employ in a regimen will take into account compatibility of the inventive compound with the additional pharmaceutical agents and/or the desired therapeutic and/or prophylactic effect to be achieved. In general, it is expected that the additional pharmaceutical agents utilized in combination be utilized at levels that do not exceed the levels at which they are utilized individually. In some embodiments, the levels utilized in combination will be lower than those utilized individually.

Exemplary additional pharmaceutical agents include, but are not limited to, anti-proliferative agents, anti-cancer agents, anti-diabetic agents, anti-inflammatory agents, immunosuppressant agents, and a pain-relieving agent. Pharmaceutical agents include small organic molecules such as drug compounds (e.g., compounds approved by the U.S. Food and Drug Administration as provided in the Code of Federal Regulations (CFR)), peptides, proteins, carbohydrates, monosaccharides, oligosaccharides, polysaccharides, nucleoproteins, mucoproteins, lipoproteins, synthetic polypeptides or proteins, small molecules linked to proteins, glycoproteins, steroids, nucleic acids, DNAs, RNAs, nucleotides, nucleosides, oligonucleotides, antisense oligonucleotides, lipids, hormones, vitamins, and cells.

Also encompassed by the invention are kits (e.g., pharmaceutical packs). The inventive kits may be useful for preventing and/or treating a proliferative disease or a non-proliferative disease, e.g., as described herein. The kits provided may comprise an inventive pharmaceutical composition or compound and a container (e.g., a vial, ampule, bottle, syringe, and/or dispenser package, or other suitable container). In some embodiments, provided kits may optionally further include a second container comprising a pharmaceutical excipient for dilution or suspension of an inventive pharmaceutical composition or compound. In some embodiments, the inventive pharmaceutical composition or compound provided in the container and the second container are combined to form one-unit dosage form.

Thus, in one aspect, provided are kits including a first container comprising a compound described herein, or a pharmaceutically acceptable salt, solvate, hydrate, tautomer, or stereoisomer thereof, or a pharmaceutical composition thereof. In certain embodiments, the kit of the disclosure includes a first container comprising a compound described herein, or a pharmaceutically acceptable salt thereof, or a pharmaceutical composition thereof. In certain embodiments, the kits are useful in preventing and/or treating a disease, disorder, or condition described herein in a subject (e.g., a proliferative disease or a non-proliferative disease). In certain embodiments, the kits further include instructions for administering the compound, or a pharmaceutically acceptable salt, solvate, hydrate, tautomer, or stereoisomer thereof, or a pharmaceutical composition thereof, to a subject to prevent and/or treat a proliferative disease or a non-proliferative disease.

### Methods of Use

Described herein are compounds useful for modulating splicing. In some embodiments, a compound of Formula (I), (II), (III), or (IV) or a pharmaceutically acceptable salt thereof may be used to alter the amount, structure, or composition of a nucleic acid (e.g., a precursor RNA, e.g., a pre-mRNA, or the resulting mRNA) by increasing or decreasing splicing at a splice site. In some embodiments, increasing or decreasing splicing results in modulating the level or structure of a gene product (e.g., an RNA or protein) produced. In some embodiments, a compound of Formula (I), (II), (III), or (IV) or a pharmaceutically acceptable salt thereof may modulate a component of the splicing machinery, e.g., by modulating the interaction with a component of the splicing machinery with another entity (e.g., nucleic acid, protein, or a combination thereof). The splicing machinery as referred to herein comprises one or more spliceosome components. Spliceosome components may comprise, for example, one or more of major spliceosome members (U1, U2, U4, U5, U6 snRNPs), or minor spliceosome members (U11, U12, U4atac, U6atac snRNPs) and their accessory splicing factors.

In another aspect, the present disclosure features a method of modifying of a target (e.g., a precursor RNA, e.g., a pre-mRNA) through inclusion of a splice site in the target, wherein the method comprises providing a compound of Formula (I), (II), (III), or (IV) or a pharmaceutically acceptable salt thereof. In some embodiments, inclusion of a splice site in a target (e.g., a precursor RNA, e.g., a pre-mRNA, or the resulting mRNA) results in addition or deletion of one or more nucleic acids to the target (e.g., a new exon, e.g. a skipped exon). Addition or deletion of one or more nucleic acids to the target may result in an increase in the levels of a gene product (e.g., RNA, e.g., mRNA, or protein).

In another aspect, the present disclosure features a method of modifying a target (e.g., a precursor RNA, e.g., a pre-mRNA, or the resulting mRNA) through exclusion of a splice site in the target, wherein the method comprises providing a Formula (I), (II), (III), or (IV) or a pharmaceutically acceptable salt thereof. In some embodiments, exclusion of a splice site in a target (e.g., a precursor RNA, e.g., a pre-mRNA) results in deletion or addition of one or more nucleic acids from the target (e.g., a skipped exon, e.g. a new exon). Deletion or addition of one or more nucleic acids from the target may result in a decrease in the levels of a gene product (e.g., RNA, e.g., mRNA, or protein). In other embodiments, the methods of modifying a target (e.g., a precursor RNA, e.g., a pre-mRNA, or the resulting mRNA) comprise suppression of splicing at a splice site or enhancement of splicing at a splice site (e.g., by more than about 0.5%, e.g., 1%, 5%, 10%, 15%, 20%, 25%, 30%, 35%, 40%, 45%, 50%, 55%, 60%, 65%, 70%, 75%, 80%, 85%, 90%, 95%, 99%, or more), e.g., as compared to a reference (e.g., the absence of a compound of Formula (I) or (II), or in a healthy or diseased cell or tissue).

The methods described herein can be used to modulate splicing, e.g., of a nucleic acid comprising a particular sequence (e.g., a target sequence). Exemplary genes encoding a target sequence (e.g., a target sequence comprising DNA or RNA, e.g., pre-mRNA) include, inter alia, ABCA4, ABCA9, ABCB1, ABCB5, ABCC9, ABCD1, ACADL, ACADM, ACADSB, ACSS2, ACTB, ACTG2, ADA, ADAL, ADAMIO, ADAMJS, ADAM22, ADAM32, ADAMTS12, ADAMTS13, ADAMTS20, ADAMTS6, ADAMTS9, ADAR, ADCY3, ADCY10, ADCY8, ADNP, ADRBK2, AFP, AGL, AGT, AHCTF1, AHR, AKAPIO, AKAP3, AKNA, ALAS1, ALS2CL, ALB, ALDH3A2, ALG6, AMBRA1, ANK3, ANTXR2, ANXA10, ANXA11, ANGPTL3, AP2A2, AP4E1, APC, APOAJ, APOB, APOC3, APOH, AR, ARID2, ARID3A, ARID3B, ARFGEF1, ARFGEF2, ARHGAP1, ARHGAP8, ARHGAP18, ARHGAP26, ARHGEF18, ARHGEF2, ARPC3, ARS2, ASH1L, ASHIL-IT1, ASNSD1, ASPM, ATAD5, ATF1, ATG4A, ATG16L2, ATM, ATN1, ATPI1C, ATP6VIG3, ATP13A5, ATP7A, ATP7B, ATR, ATXN2, ATXN3, ATXA7, ATXN10, AXIN1, B2M, B4GALNT3, BBS4, BCL2, BCL2L1, BCL2-like 11 (BIM), BCL11B, BBOXI, BCSIL, BEAN1, BHLHE40, BMPR2, BMP2K, BPTF, BRAF, BRCA1, BRCA2, BRCC3, BRSKJ, BRSK2, BTAFI, BTK, C2orf55, C4orf29, C6orf118, C9orf43, C9orf72, C10orf137, C11orf30, C11orf65, C11orf70, C11orf87, C12orf51, C13orf1, C13orf15, C14orf11, C14orf118, C15orf29, C15orf42, C15orf60, C16orf33, C16orf38, C16orf48, C18orf8, C19orf42, C1orf107, C1orf114, C1orf130, C1orf149, C1orf27, C1orf71, C1orf94, C1R, C20orf74, C21orf70, C3orf23, C4orf18, C5orf34, C8B, C8orf33, C9orf114, C9orf86, C9orf98, C3, CA11, CAB39, CACHD1, CACNA1A, CACNA1B, CACNA1C, CACNA2D1, CACNA1G, CACNA1H, CALCA, CALCOCO2, CAMK1D, CAMKK1, CAPN3, CAPN9, CAPSL, CARD11, CARKD, CASZ1, CAT, CBLB, CBX1, CBX3, CCDC102B, CCDC11, CCDC15, CCDC18, CCDC5, CCDC81, CCDC131, CCDC146, CD4, CD274, CD1B, CDC14A, CDC16, CDC2L5, CDC42BPB, CDCA8, CDH10, CDH11, CDH24, CDH8, CDH9, CDK5RAP2, CDK6, CDK8, CDK11B, CD33, CD46, CDH1, CDH23, CDK6, CDK11B, CDK13, CEBPZ, CEL, CELSR3, CENPA, CENPI, CENPT, CENTB2, CENTG2, CEPJ10, CEP170, CEP192, CETP, CFB, CFTR, CFH, CGN, CGNL1, CHAF1A, CHD9, CHIC2, CHL1, CHN1, CHM, CLEC16A, CLIC2, CLCN1, CLINT1, CLK1, CLPB, CLPTM1, CMIP, CMYA5, CNGA3, CNOT1, CNOT7, CNTN6, COG3, COL11A1, COL11A2, COL12A1, COL14A1, COL15A1, COL17A1, COL19A1, COLLA1, COL1A2, COL2A1, COL3A1, COL4A1, COL4A2, COL4A5, COL4A6, COL5A2, COL6A1, COL7AM, COL9AM, COL9A2, COL22A1, COL24A1, COL25A1, COL29A1, COLQ, COMTDI, COPA, COPB2, COPS7B, COPZ2, CPSF2, CPXM2, CR1, CRBN, CRYZ, CREBBP, CRKRS, CSE1L, CSTB, CSTF3, CT45-6, CTNNB1, CUBN, CUL4B, CUL5, CXorf41, CXXC1, CYBB, CYFIP2, CYP3A4, CYP3A43, CYP3A5, CYP4F2, CYP4F3, CYP17, CYP19, CYP24A1, CYP27A1, DAB1, DAZ2, DCBLD1, DCC, DCTN3, DCUND4, DDA1, DDEF1, DDX, DDX24, DDX4, DENND2D, DEPDC2, DES, DGAT2, DHFR, DHRS7, DHRS9, DHX8, DIP2A, DMD, DMTF, DNAH3, DNAH8, DNAIJ, DNAJA4, DNAJC3, DNAJC7, DNMT, DNTTIP2, DOCK4, DOCK5, DOCK10, DOCK11, DOT1L, DPP3, DPP4, DPY9L2P2, DR1, DSCC, DVL3, DUX4, DYNC1H1, DYSF, E2F1, E2F3, E2F8, E4F1, EBF1, EBF3, ECM2, EDEM3, EFCAB3, EFCAB4B, EFNA4, EFTUD2, EGFR, EIF3A, ELA1, ELA2A, ELF2, ELF3, ELF4, EMCN, EMD, EML5, ENO3, ENPP3, EP300, EPAS1, EPB41L5, EPHA3, EPHA4, EPHB1, EPHB2, EPHB3, EPS15, ERBB4, ERCC1, ERCC8, ERGIC3, ERMN, ERMP1, ERN1, ERN2, ESR1, ESRRG, ETS2, ETV3, ETV4, ETV5, ETV6, EVC2, EWSR1, EXO1, EXOC4, F3, F11, F13A1, F5, F7, F8, FAH, FAM13A1, FAM13B1, FAM13C1, FAM134A, FAM161A, FAM176B, FAM184A, FAM19A1, FAM20A, FAM23B, FAM65C, FANCA, FANCC, FANCG, FANCM, FANK, FAR2, FBNl, FBX015, FBX018, FBXO38, FCGBP, FECH, FEZ2, FGA, FGD6, FGFR2, FGFR1OP, FGFR1OP2, FGFR2, FGG, FGR, FIX, FKBP3, FL11, FLJ35848, FLJ36070, FLNA, FN1, FNBP1L, FOLH1, FOSL1, FOSL2, FOXKJ, FOAM1, FOXO1, FOXP4, FRAS1, FUT9, FXN, FZD3, FZD6, GAB1, GABPA, GALC, GALNT3, GAPDH, GART, GAS2L3, GATA3, GATAD2A, GBA, GBGT1, GCG, GCGR, GCK, GF11, GFM1, GH, GHR, GHV, GJA1, GLA, GLT8D1, GNA11, GNAQ, GNAS, GNB5, GOLGB, GOLT1A, GOLT1B, GPATCH1, GPR158, GPR160, GPX4, GRAMD3, GRHL1, GRHL2, GRHPR, GRIA1, GRIA3, GRIA4, GRIN2B, GRM3, GRM4, GRN, GSDMB, GSTCD, GSTO2, GTF2I, GTPBP4, HADHA, HAND2, HBA2, HBB, HCK, HDAC3, HDAC5, HDX, HEPACAM2, HERC1, HES7, HEXA, HEXB, HHEX, HIPK3, HLA-DPB1, HLA-G, HLCS, HLTF, HMBS, HMGA1, HMGCL, HNF1A, HNF1B, HNF4A, HNF4G, HNRNPH1, HOXC10, HP1BP3, HPGD, HPRT1, HPRT2, HSF1, HSF4, HSF2BP, HSPA9, HSPG2, HTT, HXA, ICA1, IDH1, IDS, IFI44L, IKBKAP, IKZF, IKZF3, IL1R2, IL5RA, IL7RA, IMMT, INPP5D, INSR, INTS3, INTU, IP04, IP08, IQGAP2, IRF2, IRF4, IRF8, IRX3, ISL1, ISL2, ITFG1, ITGA6, ITGAL, ITGB1, ITGB2, 1TGB3, ITGB4, ITIHI, ITPR2, IWS1, JAK1, JAK2, JAG1, JMJD1C, JPH3, KALRN, KAT6A, KATNAL2, KCNN2, KCNT2, KDM2A, KIAA0256, KIAA0528, KIAA0564, KIAA0586, KIAA1033, KIAA1166, KIAA1219, KIAA1409, KIAA1622, KIAA1787, KIF3B, KIF15, KIF16B, KIF5A, KIF5B, KIF9, KIN, KIR2DL5B, KIR3DL2, KIR3DL3, KIT, KLF3, KLF5, KLF7, KLF10, KLF12, KLF16, KLHL20, KLK12, KLKB1, KMT2A, KMT2B, KPNA5, KRAS, KREMENI, KRIT1, KRT5, KRTCAP2, KYNU, LICAM, L3MBTL, L3MBTL2, LACE1, LAMA1, LAMA2, LAMA3, LAMB1, LARP7, LDLR, LEF1, LENG1, LGALS3, LGMN, LHCGR, LHX3, LHX6, LIMCH1, LIMK2, LIN28B, LIN54, LMBRD1, LMBRD2, LMLN, LMNA, LMO2, LMO7, LOC389634, LOC390110, LPA, LPCAT2, LPL, LRP4, LRPPRC, LRRK2, LRRC19, LRRC42, LRWD1, LUM, LVRN, LYN, LYST, MADD, MAGI1, MAGT1, MALT1, MAP2K, MAP4K4, MAPK8IP3, MAPK9, MAPT, MARC1, MARCH5, MATN2, MBD3, MCF2L2, MCM6, MDGA2, MDM4, ASXL1, FUS, SPR54, MECOM, MEF2C, MEF2D, MEGF0, MEGF1, MEMO1, MET, MGA, MGAM, MGAT4A, MGAT5, MGC16169, MGC34774, MKKS, MIB1, MIER2, MITF, MKL2, MLANA, MLH1, MLL5, MLX, MME, MPDZ, MPI, MRAP2, MRPLH1, MRPL39, MRPS28, MRPS35, MS4A13, MSH2, MSH3, MSMB, MST1R, MTDH, MTERF3, MTF1, MTF2, MTIF2, MTHFR, MUC2, MUT, MVK, MYB, MYBL2, MYC, MYCBP2, MYH2, MYRF, MYT1, MY019, MY03A, MY09B, MYOM2, MYOM3, NAG, NARG1, NARG2, NCOA1, NDC80, NDFIP2, NEB, NEDD4, NEK1, NEK5, NEK11, NF1, NF2, NFATC2, NFE2L2, NFIA, NFIB, NFIX NFKB1, NFKB2, NFKBIL2, NFRKB, NFYA, NFYB, NIPA2, NKAIN2, NKAP, NLRC3, NLRC5, NLRP3, NLRP7, NLRP8, NLRP13, NME1, NME1-NME2, NME2, NME7, NOLI0, NOP561, NOS1, NOS2A, NOTCH1, NPAS4, NPM, NRID, NRIH3, NR1H4, NR4A3, NR5A1, NRXN1, NSMAF, NSMCE2, NT5C, NT5C2, NT5C3, NUBP1, NUBPL, NUDT5, NUMA1, NUP88, NUP98, NUP160, NUPL, OAT, OAZ1, OBFC2A, OBFC2B, OLIG2, OMA1, OPA1, OPN4, OPTN, OSBPL11, OSBPL8, OSGEPL1, OTC, OTX2, OVOL2, OXT, PA2G4, PADI4, PAH, PAN2, PAOX, PAPOLG, PARD3, PARPI, PARVB, PAWR, PAX3, PAX8, PBGD, PBRM, PBX2, PCBP4, PCCA, PCGF2, PCNX, PCOTH, PDCD4, PDE4D, PDE8B, PDE10A, PD1A3, PDH1, PDLIM5, PDXK, PDZRN3, PELI2, PDK4, PDS5A, PDS5B, PGK1, PGM2, PHACTR4, PHEX, PHKB, PHLDB2, PHOX2B, PHTF1, PIAS1, PIEZO1, PIGF, PIGN, PIGT, PIK3C2G, PIK3CA, PIK3CD, PIK3CG, PIK3R1, PIP5K1A, PITRM1, PIWIL3, PKD1, PKHD1L1, PKD2, PKIB, PKLR, PKM1, PKM2, PLAGL2, PLCB1, PLCB4, PLCG1, PLD1, PLEKHA5, PLEKHA7, PLEKHM1, PLKR, PLXNC1, PMFBP1, POLN, POLR3D, POMT2, POSTN, POU2AF, POU2F2, POU2F3, PPARA, PPFIA2, PPP1R12A, PPP3CB, PPP4C, PPP4RIL, PPP4R2, PRAME, PRC1, PRDM1, PREX1, PREX2, PRIM1, PRIM2, PRKARIA, PRKCA, PRKG1, PRMT7, PROC, PROCR, PROSC, PRODH, PROX1, PRPF40B, PRPF4B, PRRG2, PRUNE2, PSD3, PSEN1, PSMAL, PTCH1, PTEN, PTK2, PTK2B, PTPN2, PTPN3, PTPN4, PTPN1, PTPN22, PTPRD, PTPRK, PTPRM, PTPRN2, PTPRL PUS10, PVRL2, PYGM, QRSL1, RAB11FIP2, RAB23, RAF1, RALBP1, RALGDS, RB1CC1, RBL2, RBM39, RBM45, RBPJ, RBSN, REC8, RELB, RFC4, RFTJ, RFTNI, RHOA, RHPN2, RIF1, RIT1, RLN3, RMND5B, RNF11, RNF32, RNFTI, RNGTT, ROCK1, ROCK2, RORA, RP1, RP6KA3, RP11-265F1, RP13-36C9, RPAP3, RPNI, RPGR, RPL22, RPL22L1, RPS6KA6, RREBI, RRM1, RRPIB, RSK2, RTEL1, RTF1, RUFY1, RUNX1, RUNX2, RXRA, RYR3, SAAL1, SAE1, SALL4, SAT1, SATB2, SBCAD, SCN1A, SCN2A, SCN3A, SCN4A, SCN5A, SCN8A, SCNA, SCNIIA, SCO1, SCYL3, SDCJ, SDK1, SDK2, SEC24A, SEC24D, SEC31A, SELIL, SENP3, SENP6, SENP7, SERPINA1, SETD3, SETD4, SETDBI, SEZ6, SFRS12, SGCE, SGOL2, SGPLI, SH2DIA, SH3BGRL2, SH3PXD2A, SH3PXD2B, SH3RF2, SH3TC2, SHOC2, SIPAIL2, SIPAIL3, SIVA1, SKAP1, SKIV2L2, SLC6A11, SLC6A13, SLC6A6, SLC7A2, SLC12A3, SLC13A1, SLC22A17, SLC25A14, SLC28A3, SLC33A1, SLC35F6, SLC38A1, SLC38A4, SLC39A10, SLC4A2, SLC6A8, SMARCA1, SMARCA2, SMARCA5, SMARCC2, SMC5, SMN2, SMOX, SMS, SMTN, SNCAIP, SNORD86, SNRK, SNRP70, SNX5, SNX6, SOD1, SOD10, SOS, SOS2, SOX5, SOX6, SOX8, SP1, SP2, SP3, SPJ10, SPAG9, SPATA13, SPATA4, SPATS1, SPECCIL, SPDEF, SP11, SPINK5, SPP2, SPTAJ, SRF, SRM, SRP72, SSX3, SSX5, SSX9, STAG1, STAG2, STAMBPLI, STARD6, STAT1, STAT3, STAT5A, STAT5B, STAT6, STK17B, STX3, STXBPI, SUCLG2, SULF2, SUPT6H, SUPTi6H, SV2C, SYCP2, SYT6, SYCP1, SYTL3, SYTL5, TAF2, TARDBP, TBCID3G, TBCID8B, TBCID26, TBCID29, TBCEL, TBK1, TBP, TBPL1, TBR1, TBX, TCEB3, TCF3, TCF4, TCF7L2, TCFL5, TCF12, TCPIIL2, TDRD3, TEAD1, TEAD3, TEAD4, TECTB, TEK, TERF1, TERF2, TET2, TFAP2A, TFAP2B, TFAP2C, TFAP4, TFDP1, TFRC, TG, TGM7, TGSJ, THAP7, THAP12, THOC2, TIAL1, TIAM2, TIMM50, TLK2, TM4SF20, TM6SF1, TMEM27, TMEM77, TMEM156, TMEM194A, TMFJ, TMPRSS6, TNFRSFOA, TNFRSFOB, TNFRSF8, TNK2, TNKS, TNKS2, TOMIL1, TOMIL2, TOP2B, TP53, TP53INP1, TP53BP2, TP53I3, TP63, TRAF3IP3, TRAPPC2, TRIM44, TRIM65, TRIML1, TRIML2, TRPM3, TRPM5, TRPM7, TRPSJ, TSCJ, TSC2, TSHB, TSPAN7, TTC17, TTFJ, TTLL5, TTLL9, TTN, TTPAL, TTR, TUSC3, TXNDC10, UBE3A, UCK1, UGTIAJ, UHRFIBPI, UNC45B, UNC5C, USH2A, USF2, USPI, USP6, USP18, USP38, USP39, UTP20, UTP15, UTP18, UTRN, UTX, UTY, UVRAG, UXT, VAPA, VEGFA, VPS29, VPS35, VPS39, VT11A, VT11B, VWA3B, WDFY2, WDR16, WDR17, WDR26, WDR44, WDR67, WDTC1, WRN, WRNIPI, WTI, WWC3, XBPI, XRN1, XRN2, XX-FW88277, YAP1, YARS, YBX1, YGM, YY1, ZBTB18, ZBTB20, ZC3HAV1, ZC3HC1, ZC3H7A, ZDHHC19, ZEB1, ZEB2, ZFPMJ, ZFYVE1, ZFX ZIC2, ZNF37A, ZNF91, ZNF114, ZNF155, ZNF169, ZNF205, ZNF236, ZNF317, ZNF320, ZNF326, ZNF335, ZNF365, ZNF367, ZNF407, ZNF468, ZNF506, ZNF511, ZNF511-PRAPI, ZNF519, ZNF521, ZNF592, ZNF618, ZNF763, and ZWINT.

Additional exemplary genes encoding a target sequence (e.g., a target sequence comprising DNA or RNA, e.g., pre-mRNA) include genes include A1CF, A4GALT, AAR2, ABAT, ABCA11P, ZNF721, ABCA5, ABHD10, ABHD13, ABHD2, ABHD6, ACO000120.3, KRIT1, AC004076.1, ZNF772, AC004076.9, ZNF772, AC004223.3, RAD51D, ACO004381.6, AC006486.1, ERF, ACO007390.5, AC007780.1, PRKARIA, ACO007998.2, INO80C, AC009070.1, CMC2, AC009879.2, AC009879.3, ADHFE1, AC010487.3, ZNF816-ZNF321P, ZNF816, AC010328.3, AC010522.1, ZNF587B, AC010547.4, ZNF19, AC012313.3, ZNF497, AC012651.1, CAPN3, AC013489.1, DET1, AC016747.4, C2orf74, AC020907.6, FXYD3, AC021087.5, PDCD6, AHRR, AC022137.3, ZNF761, AC025283.3, NAA60, AC027644.4, RABGEF, AC055811.2, FLCN, AC069368.3, ANKDDIA, AC073610.3, ARF3, AC074091.1, GPN1, AC079447.1, LIPT1, AC092587.1, AC079594.2, TRIM59, AC091060.1, C18orf21, AC092143.3, MCIR, AC093227.2, ZNF607, AC093512.2, ALDOA, AC098588.1, ANAPCIO, AC107871.1, CALML4, AC114490.2, ZMYM6, AC138649.1, NIPAJ, AC138894.1, CLN3, AC139768.1, AC242426.2, CHDIL, ACADM, ACAP3, ACKR2, RP11-141M3.5, KRBOXI, ACMSD, ACOT9, ACP5, ACPL2, ACSBGI, ACSF2, ACSF3, ACSL1, ACSL3, ACVRI, ADAL, ADAM29, ADAMTSJO, ADAMTSL5, ADARBI, ADAT2, ADCK3, ADD3, ADGRG1, ADGRG2, ADHIB, ADIPORI, ADNP, ADPRH, AGBL5, AGPATI, AGPAT3, AGR2, AGTRI, AHDCJ, AHIJ, AHNAK, AIFMI, AIFM3, AIMP2, AK4, AKAPI, AKNADI, CLCCI, AKRJAI, AKT1, AKTISJ, AKT2, AL139011.2, PEX19, AL157935.2, ST6GALNAC6, AL358113.1, TJP2, AL441992.2, KYAT1, AL449266.1, CLCCJ, AL590556.3, LINC00339, CDC42, ALASJ, ALB, ALDH16AJ, ALDHIBI, ALDH3AJ, ALDH3B2, ALDOA, ALKBH2, ALPL, AMD1, AMICAI, AMNI, AMOTL2, AMYJB, AMY2B, ANAPCIO, ANAPCII, ANAPCJS, ANG, RNASE4, AL163636.2, ANGEL2, ANGPTLJ, ANKMYJ, ANKRD11, ANKRD28, ANKRD46, ANKRD9, ANKS3, ANKS3, RP11-127120.7, ANKS6, ANKZFJ, ANPEP, ANXA11, ANXA2, ANXA8L2, AL603965.1, AOC3, AP000304.12, CRYZLI, AP000311.1, CRYZLI, AP000893.2, RAB30, AP001267.5, ATPSMG, AP002495.2, AP003175.1, OR2AT4, AP003419.1, CLCFJ, AP005263.1, ANKRDJ2, AP006621.5, AP006621.1, APIGI, AP3M1, AP3M2, APBA2, APBBI, APLP2, APOA2, APOL1, APOL3, APTX, ARAP1, STARD10, ARF4, ARFIPJ, ARFIP2, ARFRP1, ARHGAP11A, ARHGAP33, ARHGAP4, ARHGEF10, ARHGEF3, ARHGEF35, OR2A1-AS1, ARHGEF35, OR2AJ-ASJ, ARHGEF34P, AR1D1B, ARHGEF35, OR2A20P, OR2A1-AS1, ARHGEF9, ARL1, ARL13B, ARL16, ARL6, ARMC6, ARMC8, ARMCX2, ARMCX5, RP4-769N13.6, ARMCX5-GPRASP2, BHLHB9, ARMCX5-GPRASP2, GPRASP1, ARMCX5-GPRASP2, GPRASP2, ARMCX6, ARNT2, ARPP19, ARRB2, ARSA, ART3, ASB3, GPR75-ASB3, ASCC2, ASNS, ASNS, AC079781.5, ASPSCR1, ASS1, ASUN, ATE1, ATF1, ATF7IP2, ATG13, AT1G4D, AT1G7, AT1G9A, ATM, ATOX1, ATPIB3, ATP2C, ATPSF1A, ATP5G2, ATP5J, ATP5MD, ATP5PF, ATP6AP2, ATP6VOB, ATP6VJCJ, ATP6VJD, ATP7B, ATXN1, ATXN1L, IST1, ATXN3, ATXN7L1, AURKA, AURKB, AXDND1, B3GALNT1, B3GALT5, AF064860.1, B3GALT5, AF064860.5, B3GNT5, B4GALT3, B4GALT4, B9D1, BACH1, BAIAP2, BANF1, BANF2, BAX, BAZ2A, BBIP1, BCHE, BCL2L14, BCL6, BCL9L, BCS1L, BDH1, BDKRB2, AL355102.2, BEST1, BEST3, BEX4, BHLHB9, B1D, BIN3, B1RC2, BIVM, BIVM-ERCC5, BIVM, BLCAP, BLK, BLOC1S1, RP11-644F5.10, BLOC1S6, AC090527.2, BLOC1S6, RP11-96020.4, BLVRA, BMF, BOLA1, BORCS8-MEF2B, BORCS8, BRCA1, BRD1, BRDT, BRINP3, BROX, BTBD10, BTBD3, BTBD9, BTD, BTF3L4, BTNL9, BUB1B-PAK6, PAK6, BUB3, C10orf68, C11orf1, C11orf48, C11orf54, C11orf54, AP001273.2, C11orf57, C11orf63, C11orf82, C12orf23, C12orf4, C12orf65, C12orf79, C14orf159, C14orf93, C17orf62, C18orf21, C19orf12, C19orf40, C19orf47, C19orf48, C19orf54, C1D, C1GALT1, C1QB, C1QTNF1, C1S, C1orf101, C1orf112, C1orf116, C1orf159, C1orf63, C2, C2, CFB, C20orf27, C21orf58, C2CD4D, C2orf15, LIPT1, MRPL30, C2orf80, C2orf81, C3orf14, C3orf17, C3orf18, C3orf22, C3orf33, AC104472.3, C4orf33, C5orf28, C5orf34, C6orf718, C6orf203, C6orf211, C6orf48, C7orf50, C7orf55, C7orf55-LUC7L2, LUC7L2, C8orf44-SGK3, C8orf44, C8orf59, C9, DAB2, C9orf153, C9orf9, CASBP1, CA5B, CABYR, CALCA, CALCOCO1, CALCOCO2, CALM1, CALM3, CALML4, RP11-315D16.2, CALN, CALU, CANT1, CANX, CAP1, CAPN12, CAPS2, CARD8, CARHSPI, CARNS1, CASC1, CASP3, CASP7, CBFA2T2, CBS, CBY1, CCBL1, CCBL2, RBMXL1, CCDCl2, CCDCl26, CCDCl4, CCDCl49, CCDCl50, CCDCl69-SOHLH2, CCDCl69, CCDCl71, CCDC37, CCDC41, CCDC57, CCDC63, CCDC7, CCDC74B, CCDC77, CCDC82, CCDC90B, CCDC91, CCDC92, CCNE1, CCHCR1, CCL28, CCNB11P1, CCNC, CCND3, CCNG1, CCP110, CCR9, CCT7, CCT8, CD151, CD1D, CD200, CD22, CD226, CD276, CD36, CD59, CDC26, CDC42, CDC42SE1, CDC42SE2, CDHR3, CDK10, CDK16, CDK4, CDKAL1, CDKL3, CTD-2410N18.4, CDKN1A, CDKN2A, CDNF, CEBPZOS, CELF1, CFMIP, CENPK, CEP170B, CEP250, CEP57, CEP57L1, CEP63, CERS4, CFL1, CFL2, CFLAR, CGNL1, CHCHD7, CHDIL, CHD8, CHFR, ZNF605, CHIA, CHID1, CHL1, CHM, CHMP1A, CHMP3, RNF103-CHMP3, CHRNA2, CIDEC, CIRBP, CITED1, CKLF-CMTM1, CMTM1, CKMT1B, CLDN12, CTB-13L3.1, CLDND1, AC021660.3, CLDND1, CPOX, CLHC1, CLIP1, CLUL1, CMC4, MTCP1, CNDP2, CNFN, CNOT1, CNOT6, CNOT7, CNOT8, CNR1, CNR2, CNTFR, CNTRL, COA1, COASY, COCH, COL8A1, COLCA1, COLEC11, COMMD3-BMI1, BMI1, COPS5, COPS7B, COQ8A, CORO6, COTL, COXJ4, RP4-60503.4, COX7A2, COX7A2L, COX7B2, CPA4, CPAS, CPEB1, CPNE1, AL109827.1, RBM12, CPNE1, RP1-309K20.6, RBM12, CPNE3, CPSF3L, CPTIC, CREB3L2, CREM, CRP, CRYZ, CS, AC073896.1, CS, RPIJ-977G19.10, CSAD, CSDE1, CSF2RA, CSGALNACT1, CSK, CSNK2A1, CSRNP2, CT45A4, CT45A4, CT45A5, CT45A6, CTBP2, CTCFL, CTD-2116N17.1, KIAA0101, CTD-2349B8.1, SYT17, CTD-2528L19.4, ZNF607, CTD-2619J13.8, ZNF497, CTNNA1, CTNNBIP1, CTNND1, CTPS2, CTSB, CTSL, CTTN, CUL2, CUL9, CWC15, CXorf40B, CYB561A3, CYBC1, CYLD, CYP11A1, CYP2R1, CYP4B1, CYP4F22, DAG1, DAGLB, KDELR2, DARS, DBNL, DCAFI1, DCAF8, PEX19, DCLRE1C, DCTD, DCTN1, DCTN4, DCUNID2, DDR1, DDXI1, DDX19B, AC012184.2, DDX19B, RP11-529K1.3, DDX25, DDX39B, ATP6VJG2-DDX39B, SNORD84, DDX42, DDX60L, DEDD, DEDD2, DEFA1, DEFA1B, DEFA1B, DEFA3, DENNDIC, DENND2A, DENND4B, DET1, DGKA, DGKZ, DGLUCY, DHRS4L2, DHRS9, DHX40, DIABLO, AC048338.1, DIAPH1, DICER1, DKKL1, DLG1, DLG3, DLST, DMC1, DMKN, DMTF1, DMTN, DNAJCJ4, DNAJC19, DNAL1, DNASE1L1, DNMT3A, DOC2A, DOCK8, DOKJ, DOPEY1, DPAGT1, DPP8, DRAM2, DRD2, DROSHA, DSN1, DTNA, DTX2, DTX3, DUOXI, DUOXAJ, DUS2, DUSP10, DUSP13, DUSP18, DUSP22, DYDC1, DYDC2, DYNLL1, DYNLTI, DYRKIA, DYRK2, DYRK4, RP11-500M8.7, DZIPIL, E2F6, ECHDC1, ECSIT, ECT2, EDC3, EDFM1, EDFM2, MMP24-ASJ, RP4-61404.11, EEF1AKNMT, EEF1D, EFFMP1, EFHC1, EGFL7, EHF, E124, EIFJAD, EIF2B5, EIF4G1, EIF2B5, POLR2H, EIF3E, EIF3K, EIF4E3, EIF4G1, ELFJ, ELMO2, ELMOD1, AP000889.3, ELMOD3, ELOC, ELOF1, ELOVL1, ELOVL7, ELP1, ELP6, FML3, FMP3, ENC1, ENDOV, ENO1, ENPP5, ENTHD2, ENTPD6, EP400NL, EPB41L1, EPDR1, NME8, EPHX1, EPM2A, EPN1, EPN2, EPN3, EPS8L2, ERBB3, ERC1, ERCC1, ERG, ERI2, ERI2, DCUN1D3, ERLIN2, ERMARD, ERRFI1, ESR2, RPJJ-544120.2, ESRRA, ESRRB, ESRRG, ETFA, ETFRF1, ETV1, ETV4, ETV7, EVAA, EVC2, EVX1, EXD2, EXO5, EXOC1, EXOC2, FAAP24, FABP6, FADS1, FADS2, FAHD2B, FAM107B, FAM111A, FAM111B, FAM114A1, FAM114A2, FAM115C, FAM115C, FAM115D, FAM120B, FAM133B, FAM135A, FAM153A, FAM153B, FAM154B, FAM156A, FAM156B, FAM168B, FAM172A, FAM182B, FAM192A, FAM19A2, FAM200B, FAM220A, FAM220A, AC009412.1, FAM222B, FAM227B, FAM234A, AC004754.1, FAM3C, FAM45A, FAM49B, FAM60A, FAM63A, FAM81A, FAM86B1, FAM86B2, FANC1, FANK1, FAR2, FAXC, FAXDC2, FBF1, FBH1, FBXL4, FBXO18, FBXO22, FBXO31, FBXO41, FBXO44, FBXO45, FBXW9, FCHO1, FCHSD2, FDFT1, FDPS, FER, FETUB, FGD4, FGF1, FGFR1, FGFRL1, FGL1, FHL2, FIBCDI, FIGNL1, FIGNL1, DDC, FKBP5, FKRP, FLRT2, FLRT3, FMC1, LUC7L2, FMC1-LUC7L2, FNDC3B, FOLH1, FOLR1, FOXP1, FOXK1, FOXM1, FOXO1, FOXP4, AC097634.4, FOXRED1, FPR1, FPR2, FRG1B, FRS2, FTO, FTSJ1, FUK, FUT10, FUT3, FUT6, FXYD3, FZD3, G2E3, GAA, GABARAPL1, GABPB1, GABRA5, GAL3ST1, GALE, GALNTII, GALNTJ4, GALNT6, GAPVD1, GARNL3, GAS2L3, GAS8, GATA1, GATA2, GATA4, GBA, GCNTI, GDPD2, GDPD5, GFMIN7, MARK4, GFMIN8, GGA3, GGACT, AL356966.1, GGPSI, GHRL, GID8, GIGYF2, GIMAP8, GIPCI, GJBI, GJB6, GLBIL, GIi, GLT8D1, GMFG, GMPR2, GNAI2, GNAQ, GNB1, GNB2, GNE, GNG2, GNGT2, GNPDA1, GNPDA2, GOLGA3, CHFR, GOLGA4, GOLPH3L, GOLTIB, GPBPILI, GPERI, GPRJJ6, GPR141, EPDR1, GPR155, GPR161, GPR56, GPR63, GPR75-ASB3, ASB3, GPR85, GPSM2, GRAMDIB, GRBIO, GRB7, GREW2, GRIA2, GSDMB, GSEI, GSN, GSTA4, GSTZJ, GTDC1, GTF2H1, GTF2H4, VARS2, GTF3C2, GUCYIA3, GUCYIB3, GUK1, GULP1, GYPC, GYSI, GZFJ, HAGH, HA02, HAPLN3, HAVCR1, HAX1, HBG2, AC104389.4, HBG2, AC104389.4, HBEI, HBG2, AC104389.4, HBEI, OR51B5, HBG2, HBEJ, AC104389.28, HBSIL, HCFCIRI, HCK, HDAC2, HDAC6, HDAC7, HDLBP, HEATR4, HECTD4, HEXUf2, HHAT, HHATL, CCDCl3, HINFP, HIRA, C22orf39, HIVEP3, HJV, HKRI, HLF, HMBOXI, HMGAJ, HMGB3, HMGCR, HMGN4, HMOX2, HNRNPC, HNRNPD, HNRNPH1, HNRNPH3, HNRNPR, HOMER3, HOPX, HOXA3, HOXB3, HOXB3, HOXB4, HOXC4, HOXD3, HOXD3, HOXD4, HPCALI, HPS4, HPS5, HRHI, HS3ST3A1, HSH2D, HSP90AA1, HSPD1, HYTT, HUWEI, HYOU1, IAHI, ICAIL, ICAM2, ICE2, ICK, IDH2, IDH3G, IDS, IF127, IF144, IFT20, IFT22, IFT88, IGF2, INS-IGF2, IGF2BP3, IGFBP6, IKBKAP, IKBKB, ILII, IL18BP, IL18RAP, ILIRAP, ILIRLI, IL18R1, ILIRN, IL32, IL411, NUP62, AC011452.1, IL411, NUP62, CTC-326K19.6, IL6ST, ILVBL, IMMPIL, IMPDH1, INCA1, ING1, INIP, INPP1, INPPS1, INPPSK, INSIG2, INTS11, INTS12, INTS14, IP6K2, IP6K3, IPOIJ, LRRC70, IQCE, IQGAP3, IRAK4, IRF3, IRF5, IRF6, ISG20, IST1, ISYNAI, ITFG2, ITGBIBPI, ITGB7, ITIH4, RP5-966M1.6, ITPRIPL1, JADEJ, JAK2, JARID2, JDP2, KANK1, KANK1, RP11-31F19.1, KANK2, KANSLIL, KAT6A, KBTBD2, KBTBD3, KCNAB2, KCNE3, KCNG1, KCNJ16, KCNJ9, KCNMB2, AC117457.1, LINC01014, KCTD20, KCTD7, RABGEF1, KDMIB, KDM4A, AL451062.3, KHNYN, KIAA0040, KIAA0125, KIAA0196, KIAA0226L, PPPIR2P4, KIAA0391, KIAA0391, AL121594.1, KIAA0391, PSMA6, KIAA0753, KIAA0895, KIAA0895L, KIAA1191, KIAA1407, KIAA1841, C2orf74, KIF12, KIF14, KIF27, KIF9, KIFC3, KIN, KIRRELI, KITLG, KLCI, APOPTI, AL139300.1, KLC4, KLHDC4, KLHDC8A, KLHL13, KLHL18, KLHL2, KLHL24, KLHL7, KLKI1, KLK2, KLK5, KLK6, KLK7, KNOPI, KRBA2, AC135178.2, KRBA2, RP11-849F2.7, KRIT1, KRT15, KRT8, KTN1, KXD1, KYAT3, RBMXL1, KYNU, L3MBTL1, LACCI, LARGE, LARP4, LARP7, LA 72, LBHD1, LCA5, LCASL, LCTL, LEPROTL, LGALS8, LGALS9C, LGMN, LHFPL2, LIG4, LIMCH1, LIMK2, LIMS2, LINC00921, ZNF263, LIPF, LLGL2, LMAN2L, LMCD1, LMF1, RP11-161M6.2, LMO1, LM03, LOXHD1, LPAR1, LPAR2, LPAR4, LPAR5, LPAR6, LPHN1, LPIN2, LPIN3, LPP, LRFN5, LRIF1, LRMP, LRRC14, LRRC20, LRRC24, C8orf82, LRRC39, LRRC42, LRRC48, LRRC4C, LRRC8A, LRRC8B, LRRD1, LRTOMT, LRTOMT, AP000812.5, LSM7, LTB4R, LTBP3, LUC7L2, FMC1-LUC7L2, LUC7L3, LUZP1, LYGI, LYL1, LYPD4, LYPD6B, LYRMI, LYRM5, LYSMD4, MACCl, MADILI, MADILI, AC069288.1, MAEA, MAFF, MAFG, MAFK, MAGEA12, CSAG4, MAGEA2, MAGEA2B, MAGEA4, MAGEBI, MAGOHB, MAN2A2, MANBAL, MAOB, MAP2K3, MAP3K7CL, MAP3K8, MAP7, MAP9, MAPK6, MAPK7, MAPK8, MAPKAP1, 10-Mar, 7-Mar, 8-Mar, MARK2, MASPI, MATK, MA TR3, MA TR3, SNHG4, MB, MBD5, MBNL1, MBOAT7, MCC, MCFD2, MCM9, MCOLN3, MCRS1, MDC1, MDGA2, MDH2, MDM2, MEl, MEAK7, MECR, MED4, MEF2A, MEF2B, BORCS8-MEF2B, MEF2BNB-MEF2B, MEF2B, MEF2BNB, MEF2C, MEF2D, MEGF10, MEIJ, MEIS2, MELK, MET, ME BTL13, METTL23, MFF, MFN2, MFSD2A, MGST3, MIB2, MICALI, MICAL3, MICOS10, NBL1, MICOS10-NBL1, MID1, MINA, MINOSJ-NBL1, MINOSI, MIOS, MIPOLI, MIS12, MKLNI, MKNKI, MKNKI, MOB3C, MLF2, MLH1, MMPJ7, MOBP, MOCSJ, MOGS, MOK, MORF4L1, MPC1, MPC2, MPG, MPI, MPP1, MPP2, MPPEJ, MPST, MRAS, MRO, MROHI, MROH7-TTC4, MROH7, MRPL14, MRPL24, MRPL33, BABAM2, MRPL33, BRE, MRPL47, MRPL48, MRPL55, MR, MRTFA, MRTFB, MRVII, MS4AJ, MS4A15, MS4A3, MS4A6E, MS4A7, MS4A14, MSANTD3, MSANTD4, MSH5, MSH5-SAPCD1, MSL2, MSRB3, MSS51, MTCP1, CMC4, MTERF, MTERFJ, MTERF3, MTERFD2, MTERFD3, MTF2, MTG2, MTHFD2, MTHFD2L, MTIF2, MTIF3, MTMR10, MTRFJ, MTRR, MTUS2, MUTYH, MVK, MXJ, MX2, MYHIO, MYL12A, MYB, MYD88, MYL5, MYIJP, MYNN, MYO15A, MYOJB, MYOM2, MZFJ, N4BP2L2, NAA60, NABJ, NAEJ, NAGK, NAPILI, NAPIL4, NAPG, NARFL, NARG2, NATi, NATIO, NBPFI1, W12-3658N16.1, NBPF12, NBPF15, NBPF24, NBPF6, NBPF9, NBRI, NCAPG2, NCBP2, NCEHi, NCOA1, NCOA4, NDCI, NDRG1, NDRG2, NDRG4, NDSTI, NDUFAF6, NDUFB2, NDUFC1, NDUFS1, NDUFS8, NDUFVJ, NEDD1, NEIL1, NEIL2, NEK10, NEK11, NEK6, NEK9, NELFA, NEU4, NFAT5, NFE2, NFE2L2, AC019080.1, NFRKB, NFYA, NFYC, NIF3L1, NIPA2, NKIRAS1, NKX2-1, NLRC3, NME1, NME1-NME2, NME2, NME1-NME2, NME2, NME4, NME6, NME9, NOD1, NOL10, NOL8, NONO, NPAS1, NPIPA8, RP11-1212A22.1, NPIPB3, NPIPB4, NPIPB9, NPL, NPM1, NPPA, NQO2, NRIH3, NR2C2, NR2F2, NR4A1, NRDC, NREP, NRFI, NRG4, NRIPI, NSD2, NSDHL, NSGI, NSMCE2, NSRPI, NT5C2, NTF4, NTMT1, NTNG2, NUBP2, NUCB2, NUDT1, NUDT2, NUDT4, NUF2, NUMBL, NUP50, NUP54, NUP85, NVL, NXFI, NXPEJ, NXPE3, OARD1, OAT, OAZ2, OCIAD1, OCLN, ODF2, OGDHL, OGFOD2, AC026362.1, OGFOD2, RP11-197N18.2, OLA1, OPRL1, OPTN, OR2H1, ORA12, ORMDL1, ORMDL2, ORMDL3, OSBPL2, OSBPL3, OSBPL5, OSBPL9, OSERI, OSGINI, OSR2, P2RX4, P2RY2, P2RY6, P4HA2, PABPCI, PACRGL, PACSIN3, PADII, PAIP2, PAKi, PAK3, PAK4, PAK7, PALB2, PANK2, PAQR6, PARPii, PARVG, PASK, PAX6, PBRMI, PBXIPI, PCBP3, PCBP4, ACii5284.1, PCBP4, RP11-155D18.14, RP11-155D18.12, PCGF3, PCGF5, PCNP, PCSK9, PDCDIO, PDCD6, AHRR, PDDCJ, PDGFRB, PDIA6, PDIKIL, PDLIM7, PDPI, PDPKI, PDPN, PDZDiI, PEA15, PEX2, PEX5, PEXSL, PFKM, PFN4, PGAP2, PGAP2, AC090587.2, PGAP3, PGM3, PGPEPI, PHB, PHC2, PHF20, PHF21A, PHF23, PHKB, PHLDB1, PHOSPHOJ, PHOSPHO2, KLHL23, P14 KB, PIAS2, PICALM, PIFI, PIGN, PIGO, PIGT, PIK3CD, PILRB, STAG3L5P-PVRIG2P-PILRB, PIPSK1B, PIR, PISD, PIWIL4, FUT4, PKD2, PKIA, PKIG, PKM, PKN2, PLAIA, PLA2G2A, PLA2G5, PL42G7, PL4C8, PLAGL1, PLD1, PLD3, PLEKHA1, PLEKHA2, PLEKHA6, PLEKHG5, PLINI, PLSI, PLS3, PLSCR1, PLSCR2, PLSCR4, PLXNB1, PLXNB2, PMP22, PMS1, PNISR, PNKP, AKT1S1, PNMT, PNPLA4, PNPLA8, PNPO, PNRC1, POCIB, POFUTI, POLB, POLDI, POLH, POLI, POLL, POLRIB, POM121, POMI21C, AC006014.7, POMI21C, AC211429.1, POMC, POMT1, POPJ, PORCN, POU5F1, PSORSIC3, PPARD, PPARG, PPHLN1, PPIL3, PPIL4, PPM1A, PPMIB, AC013717.1, PPP1CB, PPP1R11, PPPIR13L, PPP1R26, PPP1R9A, PPP2R2B, PPP3CA, PPP6R1, PPP6R3, PPT2, PPT2-EGFL8, EGFL8, PPWD1, PRDM2, PRDM8, PRELID3A, PREPL, PRICKLE1, PRKAG1, PRMT2, PRAT5, PRMT7, PROMJ, PRPS1, PRPSAP2, PRR14L, PRR15L, PRR5, PRR5-ARHGAP8, PRRL, PRR7, PRRC2B, PRRT4, PRSS50, PRSS45, PRSS44, PRUNE, PRUNE1, PSEN1, PSMA2, PSMF1, PSORSIC1, PSPH, PSRC1, PTBP3, PTHL H, PTK2, PTPDC1, PTPRM, PUF60, PUM2, PUS1, PUS10, PXN, PXYLP1, PYCR1, QRICH1, R3HCCIL, R3HDM2, RAB17, RAB23, RAB3A, RAB3D, TMEM205, RAB4B-EGLN2, EGLN2, ACO008537.1, RAB5B, RAB7L1, RABL2A, RABL2B, RABL5, RACGAP1, RAD17, RAD51L3-RFFL, RAD51D, RAD52, RAE1, RAIH4, RA12, RALBP1, RAN, RANGAPI, RAPIA, RAPIB, RAPIGAP, RAPGEF4, RAPGEFL1, RASGRP2, RASSF1, RBCK1, RBM12B, RBM14, RBM4, RBM14-RBM4, RBM23, RBM4, RBM14-RBM4, RBM47, RBM7, AP002373.1, RBM7, RP11-212D19.4, RBMS2, RBMYJE, RBPJ, RBPMS, RBSN, RCBTB2, RCC1, RCC1, SNHG3, RCCD1, RECQL, RELL2, REPINI, AC073111.3, REPINI, ZNF775, RERI, RERE, RFWD3, RFX3, RGL2, RGMB, RGSI1, RGS3, RGS5, AL592435.1, RHBDD1, RHNO1, TULP3, RHOC, AL603832.3, RHOC, RP11-426L16.10, RHOH, RIC8B, RMKLB, RINI, RIPK2, RIT1, RLIM, RNASE4, ANG, AL163636.6, RNASEK, RNASEK-CJ7orf49, RNF11, RNF123, RNF13, RNF14, RNF185, RNF216, RNF24, RNF32, RNF34, RNF38, RNF4, RNF44, RNH1, RNMT, RNPS1, RO60, ROPN1, ROPNIB, ROR2, RP1-102H19.8, C6orf163, RP1-283E3.8, CDKIIA, RP11-120M18.2, PRKARIA, RP11-133K1.2, PAK6, RP11-164J13.1, CAPN3, RP11-21J18.1, ANKRD12, RP11-322E11.6, INO80C, RP11-337C18.10, CHDIL, RP11-432B6.3, TRIM59, RP11-468E2.4, IRF9, RP11-484M3.5, UPKIB, RP11-517H2.6, CCR6, RP11-613M10.9, SLC25A51, RP11-659G9.3, RAB30, RP11-691N7.6, CTNND1, RP11-849H4.2, RP11-896J10.3, NKX2-1, RP11-96020.4, SQRDL, RP11-986E7.7, SERPINA3, RP4-769N13.6, GPRASP1, RP4-769N13.6, GPRASP2, RP4-798P15.3, SEC16B, RP5-1021120.4, ZNF410, RP6-109B7.3, FLJ27365, RPE, RPH3AL, RPL15, RPL17, RPL17-C18orf32, RPL17, RPL23A, RPL36, HSDI1BIL, RPP38, RPS20, RPS27A, RPS3A, RPS6KA3, RPS6KC1, RPS6KL1, RPUSD1, RRAGD, RRAS2, RRBP1, RSLID1, RSRC2, RSRP1, RUBCNL, RUNXITI, RUVBL2, RWDD1, RWDD4, S100A13, AL162258.1, S100A13, RP1-178F15.5, S100A16, S100A4, S100A3, S100A6, S100PBP, SAA1, SACMIL, SAMD4B, SARA, SARAF, SARNP, RP11-76217.5, SCAMP5, SCAP, SCAPER, SCFD1, SCGB3A2, SCIN, SCML1, SCNNID, SCO2, SCOC, SCRNI, SDC2, SDC4, SEC13, SEC14L1, SEC14L2, SEC22C, SEC23B, SEC24C, SEC61G, SFMA4A, SFMA4C, SFMA4D, SFMA6C, SENP7, SEPPI, 11-Sep, 2-Sep, SERGEF, AC055860.1, SERPI, SERPINA1, SERPINA5, SERPINB6, SERPINGI, SERPINHI, SERTAD3, SETD5, SFMBTi, AC096887.1, SFTPA1, SFTPA2, SFXN2, SGCD, SGCE, SGK3, SGK3, C8orf44, SH2B1, SH2D6, SH3BP1, Z83844.3, SH3BP2, SH3BP5, SH3D19, SH3YL1, SHC1, SHISA5, SHMT1, SHMT2, SHOC2, SHROOMI, SIGLEC5, SIGLEC14, SIL1, SIN3A, SIRT2, SIRT6, SKPI, STAT4, AC104109.3, SLAIN1, SLCIOA3, SLC12A9, SLC14A1, SLCO6A6, SLCIA2, SLCIA6, SLC20A2, SLC25A18, SLC25A19, SLC25A22, SLC25A25, SLC25A29, SLC25A30, SLC25A32, SLC25A39, SLC25A44, SLC25A45, SLC25A53, SLC26A11, SLC26A4, SLC28AJ, SLC29AJ, SLC2A14, SLC2A5, SLC2A8, SLC35B2, SLC35B3, SLC35C2, SLC37AW, SLC38A, SLC38A11, SLC39A13, SLC39A14, SLC41A3, SLC44A3, SLC4A7, SLC4A8, SLCSA10, SLCSAJJ, SLC6A1, SLC6A12, SLC6A9, SLC7A2, SLC7A6, SLC7A7, SLCOJA2, SLCOICI, SLCO2B1, SLFN11, SLFN12, SLFNL1, SLMOI, SLTM, SLU7, SMAD2, SMAP2, SMARCA2, SMARCEJ, AC073508.2, SMARCEJ, KRT222, SMC6, SMG7, SMWM22, SMOX; SMPDL3A, SMTN, SMUJ, SMUGJ, SNAP25, SNCA, SNRK, SNRPC, SNRPD1, SNRPD2, SNRPN, SNRPN, SNURF, SNUPN, SNXI1, SNX16, SNX17, SOAT1, SOHLH2, CCDCl69-SOHLH2, CCDCl69, SORBS1, SORBS2, SOX5, SP2, SPART, SPATA20, SPATA21, SPATS2, SPATS2L, SPDYE2, SPECCI, SPECCIL, SPECCIL-ADORA2A, SPECCIL-ADORA2A, ADORA2A, SPEG, SPG20, SPG21, SPIDR, SPIN1, SPOCD1, SPOP, SPRR2A, SPRR2B, SPRR2E, SPRR2B, SPRR2F, SPRR2D, SPRR3, SPRY1, SPRY4, SPTBN2, SRC, SRGAPI, SRP68, SRSFIJ, SSXJ, SSX2IP, ST3GAL4, ST3GAL6, ST5, ST6GALNAC6, ST7L, STAC3, STAG1, STAG2, STAMBP, STAMBPLI, STARD3NL, STAT6, STAUJ, STAU2, AC022826.2, STAU2, RP11-463D19.2, STEAP2, STEAP3, STIL, STK25, STK33, STK38L, STK40, STMNJ, STONI, STONI-GTF2AJL, STRAP, STRBP, STRC, AC011330.5, STRC, CATSPER2, STRC, CATSPER2, AC011330.5, STRC, STRCP1, ST3A, STX16-NPEPLI, NPEPLI, STX5, STX6, STX8, STXBP6, STYKI, SULTIAJ, SULTIA2, SUMF2, SUNJ, SUN2, SUN2, DNAL4, SUOX, SUPT6H, SUV39H2, SV2B, SYBU, SYNCRIP, SYNJ2, SYTI, SYTL4, TAB2, TACCI, TADA2B, TAFIC, TAF6, AC073842.2, TAF6, RP11-506M12.1, TAF9, TAGLN, TANK, TAPSARI, PSMB9, TAPTI, TATDN1, TAZ, TBCJDI, TBCID12, HELLS, TBCJD15, TBCID3H, TBCID3G, TBCID5, TBCJD5, SA TB1, TBCA, TBCEL, TBCEL, AP000646.1, TBLIXRI, TBP, TBX5, TBXAS1, TCAF1, TCEA2, TCEAL4, TCEAL8, TCE4L9, TCFANC, TCEB1, TCF19, TCF25, TCF4, TCP1, TCPIOL, AP000275.65, TCPHJ, TCPIIL2, TCTNI, TDG, TDP1, TDRD7, TE7AD2, TECR, TENC1, TENT4A, TEX264, TEX30, TEX37, TFDP1, TFDP2, TFEB, TFG, TFPI, TF, TFPI, TGIFI, THAP6, THBS3, THOC5, THRAP3, THUMPD3, TIAL1, TIMM9, TIMP1, TIRAP, TJAPI, TJP2, TK2, TLDCJ, TLE3, TLE6, TLN1, TLR10, TM9SF1, TMBAIH, TMBIM4, TMBIM6, TMC6, TMCCJ, TMCO4, TMEM126A, TMEf139, TMEf150B, TMEM155, TM1AfJ61B, TM1AfJ64, TMEFM168, TMEM169, TMEM175, TMEM176B, TM1 kf182, TMEAfJ99, CTB-96E2.3, TMEM216, TMEM218, TMEM230, TMEM263, TMEM45A, TMEM45B, TMEM62, TMEM63B, TMEM66, TMEM68, TMEM98, TMEM9B, TMPRSSIID, TMPRSS5, TMSB15B, TMTC4, TMUB2, TMX2-CTNND1, RP11-691N7.6, CTN7ND1, TNFAIP2, TNFAIP8L2, SCNMI, TNFRSFIOC, TNFRSFI9, TNFRSF8, TNFSF12-TNFSF13, TNFSF12, TNFSF13, TNFSF12-TNFSF13, TNFSF13, TNIP1, TNK2, TNNT1, TNRC18, TNS3, TOB2, TOM1L1, TOP1MT, TOP3B, TOX2, TP53, RP11-199F11.2, TP53I11, TP53INP2, TPCNI, TPM3P9, AC022137.3, TPTI, TRA2B, TRAF2, TRAF3, TRAPPC12, TRAPPC3, TREH, TREX1, TREX2, TRIB2, TR&M3, TR&M36, TRIM39, TRIM46, TR&M6, TRIM6-TRIM34, TR&M6-TRIM34, TRIM34, TMU66, TRM73, TRIT1, TRMT10B, TRMT2B, TRMT2B-ASJ, TRNT1, TRO, TROVE2, TRPS1, TRPT1, TSC2, ISGA10, TSPAN14, TSPAN3, TSPAN4, TSPAN5, TSPAN6, TSPAN9, TSPO, T7C12, 1TC23, TTC3, T7C39A, T7C39C, TTLL1, TTLL7, TTPAL, TUBDI, TWNK, TXNL4A, TXNL4B, TXNRD1, 7YK2, U2AF1, UBA2, UBA52, UBAP2, UBE2D2, UBE2D3, UBE2E3, UBE2I, UBE2J2, UBE3A, UBL7, UBXNI, UBXN7, UGDH, UGGT1, UGP2, UMAD1, ACO007161.3, UNC45A, UQCCI, URGCP-MRPS24, URGCP, USMG5, USP16, USP21, USP28, USP3, USP33, USP35, USP54, USP9Y, USPL1, UTP15, VARS2, VASH2, VAV3, VDAC1, VDAC2, VDR, VEZT, VGF, VILI, VILL, VIPRI, VPS29, VPS37C, VPS8, VPS9D1, VRK2, VWA1, VWASA, WARS, WASFI, WASHC5, WBP5, WDHD1, WDPCP, WDR37, WDR53, WDR6, WDR72, WDR74, WDR81, WDR86, WDYHVI, WFDC3, WHSCI, WIPFJ, WSCD2, WWP2, XAGEIA, XAGEIB, XKR9, XPNPEPI, XRCC3, XRN2, XXYLTI, YIFIA, YIFIB, YIPFJ, YIPF5, YPEL5, YWHAB, YWHAZ, YYIAPI, ZBTB1, ZBTB14, ZBTB18, ZBTB20, ZBTB21, ZBTB25, ZBTB33, ZBTB34, ZBTB38, ZBTB43, ZBTB49, ZBTB7B, ZBTB7C, ZBTB8OS, ZC3H11A, ZBED6, ZC3H13, ZCCHC17, ZCCHC7, ZDHHCII, ZDHHC13, ZEB2, ZFAND5, ZFAND6, ZFP1, ZFP62, ZFX, ZFYVE16, ZFYVE19, ZFYVE20, ZFYVE27, ZHX2, AC016405.1, ZHX3, ZIKI, ZIM2, PEG3, ZKSCAN1, ZKSCAN3, ZKSCAN8, ZMAT3, ZMAAT5, ZIZ2, ZWYM6, ZWYNDI1, ZNF10, AC026786.1, ZNF133, ZNF146, ZNF16, ZNF177, ZNF18, ZNF200, ZNF202, ZNF211, ZNF219, ZNF226, ZNF227, ZNF23, AC010547.4, ZNF23, AC010547.9, ZNF239, ZNF248, ZNF25, ZNF253, ZNF254, ZNF254, AC092279.1, ZNF263, ZNF274, ZNF275, ZNF28, ZNF468, ZNF283, ZNF287, ZNF3, ZNF320, ZNF322, ZNF324B, ZNF331, ZNF334, ZNF34, ZNF350, ZNF385A, ZNF395, FBXO16, ZNF415, ZNF418, ZNF43, ZNF433-AS1, ACO008770.4, ZNF438, ZNF444, ZNF445, ZNF467, ZNF480, ZNF493, ZNF493, CTD-2561J22.3, ZNF502, ZNF507, ZNF512, AC074091.1, ZNF512, RP11-158113.2, ZNF512B, ZNF512B, SAMD10, ZNF521, ZNF532, ZNF544, AC020915.5, ZNF544, CTD-3138B18.4, ZNF559, ZNF177, ZNF562, ZNF567, ZNF569, ZNF570, ZNF571-AS1, ZNF540, ZNF577, ZNF580, ZNF581, ZNF580, ZNF581, CCDCl06, ZNF600, ZNF611, ZNF613, ZNF615, ZNF619, ZNF620, ZNF639, ZNF652, ZNF665, ZNF667, ZNF668, ZNF671, ZNF682, ZNF687, ZNF691, ZNF696, ZNF701, ZNF706, ZNF707, ZNF714, ZNF717, ZNF718, ZNF720, ZNF721, ZNF730, ZNF763, ZNF780B, AC005614.5, ZNF782, ZNF786, ZNF79, ZNF791, ZNF81, ZNF83, ZNF837, ZNF839, ZNF84, ZNF845, ZNF846, ZNF865, ZNF91, ZNF92, ZNHIT3, ZSCAN21, ZSCAN25, ZSCAN30, and ZSCAN32.

In some embodiments, the gene encoding a target sequence comprises the HTT gene. In some embodiments, the gene encoding a target sequence comprises the SMN2 gene.

Exemplary genes that may be modulated by the compounds of Formula (I), (II), (III), or (IV) described herein may also include, inter alia, AC005258.1, AC005943.1, AC007849.1, AC008770.2, AC010487.3, AC011477.4, AC012651.1, AC012531.3, AC034102.2, AC073896.4, AC104472.3, AL109811.3, AL133342.1, AL137782.1, AL157871.5, AF241726.2, AL355336.1, AL358113.1, AL360181.3, AL445423.2, AL691482.3, AP001267.5, RF01169, and RF02271.

The compounds described herein may further be used to modulate a sequence comprising a particular splice site sequence, e.g., an RNA sequence (e.g., a pre-mRNA sequence). In some embodiments, the splice site sequence comprises a 5′ splice site sequence. In some embodiments, the splice site sequence comprises a 3′ splice site sequence. Exemplary gene sequences and splice site sequences (e.g., 5′ splice site sequences) include

(SEQ ID NO: 1)
AAAgcaaguu,
(SEQ ID NO: 2)
AAAguaaaaa,
(SEQ ID NO: 3)
AAAguaaaau,
(SEQ ID NO: 4)
AAAguaaagu,
(SEQ ID NO: 5)
AAAguaaaua,
(SEQ ID NO: 6)
AAAguaaaug,
(SEQ ID NO: 7)
AAAguaaauu,
(SEQ ID NO: 8)
AAAguaacac,
(SEQ ID NO: 9)
AAAguaacca,
(SEQ ID NO: 10)
AAAguaacuu,
(SEQ ID NO: 11)
AAAguaagaa,
(SEQ ID NO: 12)
AAAguaagac,
(SEQ ID NO: 13)
AAAguaagag,
(SEQ ID NO: 14)
AAAguaagau,
(SEQ ID NO: 15)
AAAguaagca,
(SEQ ID NO: 16)
AAAguaagcc,
(SEQ ID NO: 17)
AAAguaagcu,
(SEQ ID NO: 18)
AAAguaagga,
(SEQ ID NO: 19)
AAAguaaggg,
(SEQ ID NO: 20)
AAAguaaggu,
(SEQ ID NO: 21)
AAAguaagua,
(SEQ ID NO: 22)
AAAguaaguc,
(SEQ ID NO: 23)
AAAguaagug,
(SEQ ID NO: 24)
AAAguaaguu,
(SEQ ID NO: 25)
AAAguaaucu,
(SEQ ID NO: 26)
AAAguaauua,
(SEQ ID NO: 27)
AAAguacaaa,
(SEQ ID NO: 28)
AAAguaccgg,
(SEQ ID NO: 29)
AAAguacuag,
(SEQ ID NO: 30)
AAAguacugg,
(SEQ ID NO: 31)
AAAguacuuc,
(SEQ ID NO: 32)
AAAguacuug,
(SEQ ID NO: 33)
AAAguagcuu,
(SEQ ID NO: 34)
AAAguaggag,
(SEQ ID NO: 35)
AAAguaggau,
(SEQ ID NO: 36)
AAAguagggg,
(SEQ ID NO: 37)
AAAguaggua,
(SEQ ID NO: 38)
AAAguaguaa,
(SEQ ID NO: 39)
AAAguauauu,
(SEQ ID NO: 40)
AAAguauccu,
(SEQ ID NO: 41)
AAAguaucuc,
(SEQ ID NO: 42)
AAAguaugga,
(SEQ ID NO: 43)
AAAguaugua,
(SEQ ID NO: 44)
AAAguaugug,
(SEQ ID NO: 45)
AAAguauguu,
(SEQ ID NO: 46)
AAAguauugg,
(SEQ ID NO: 47)
AAAguauuuu,
(SEQ ID NO: 48)
AAAgucagau,
(SEQ ID NO: 49)
AAAgucugag,
(SEQ ID NO: 50)
AAAgugaaua,
(SEQ ID NO: 51)
AAAgugagaa,
(SEQ ID NO: 52)
AAAgugagac,
(SEQ ID NO: 53)
AAAgugagag,
(SEQ ID NO: 54)
AAAgugagau,
(SEQ ID NO: 55)
AAAgugagca,
(SEQ ID NO: 56)
AAAgugagcu,
(SEQ ID NO: 57)
AAAgugaggg,
(SEQ ID NO: 58)
AAAgugagua,
(SEQ ID NO: 59)
AAAgugaguc,
(SEQ ID NO: 60)
AAAgugagug,
(SEQ ID NO: 61)
AAAgugaguu,
(SEQ ID NO: 62)
AAAgugcguc,
(SEQ ID NO: 63)
AAAgugcuga,
(SEQ ID NO: 64)
AAAguggguc,
(SEQ ID NO: 65)
AAAguggguu,
(SEQ ID NO: 66)
AAAgugguaa,
(SEQ ID NO: 67)
AAAguguaug,
(SEQ ID NO: 68)
AAAgugugug,
(SEQ ID NO: 69)
AAAguguguu,
(SEQ ID NO: 70)
AAAguuaagu,
(SEQ ID NO: 71)
AAAguuacuu,
(SEQ ID NO: 72)
AAAguuagug,
(SEQ ID NO: 73)
AAAguuaugu,
(SEQ ID NO: 74)
AAAguugagu,
(SEQ ID NO: 75)
AAAguuugua,
(SEQ ID NO: 76)
AACguaaaac,
(SEQ ID NO: 77)
AACguaaagc,
(SEQ ID NO: 78)
AACguaaagg,
(SEQ ID NO: 79)
AACguaagca,
(SEQ ID NO: 80)
AACguaaggg,
(SEQ ID NO: 81)
AACguaaguc,
(SEQ ID NO: 82)
AACguaagug,
(SEQ ID NO: 83)
AACguaaugg,
(SEQ ID NO: 84)
AACguaguga
(SEQ ID NO: 85)
AACguaugua,
(SEQ ID NO: 86)
AACguauguu,
(SEQ ID NO: 87)
AACgugagca,
(SEQ ID NO: 88)
AACgugagga,
(SEQ ID NO: 89)
AACgugauuu,
(SEQ ID NO: 90)
AACgugggau,
(SEQ ID NO: 91)
AACgugggua,
(SEQ ID NO: 92)
AACguguguu,
(SEQ ID NO: 93)
AACguuggua,
(SEQ ID NO: 94)
AAGgcaaauu,
(SEQ ID NO: 95)
AAGgcaagag,
(SEQ ID NO: 96)
AAGgcaagau,
(SEQ ID NO: 97)
AAGgcaagcc,
(SEQ ID NO: 98)
AAGgcaagga,
(SEQ ID NO: 99)
AAGgcaaggg,
(SEQ ID NO: 100)
AAGgcaagug,
(SEQ ID NO: 101)
AAGgcaaguu,
(SEQ ID NO: 102)
AAGgcacugc,
(SEQ ID NO: 103)
AAGgcagaaa,
(SEQ ID NO: 104)
AAGgcaggau,
(SEQ ID NO: 105)
AAGgcaggca,
(SEQ ID NO: 106)
AAGgcaggga,
(SEQ ID NO: 107)
AAGgcagggg,
(SEQ ID NO: 108)
AAGgcaggua,
(SEQ ID NO: 109)
AAGgcaggug,
(SEQ ID NO: 110)
AAGgcaucuc,
(SEQ ID NO: 111)
AAGgcaugcu,
(SEQ ID NO: 112)
AAGgcaugga,
(SEQ ID NO: 113)
AAGgcauguu,
(SEQ ID NO: 114)
AAGgcauuau,
(SEQ ID NO: 115)
AAGgcgagcu,
(SEQ ID NO: 116)
AAGgcgaguc,
(SEQ ID NO: 117)
AAGgcgaguu,
(SEQ ID NO: 118)
AAGgcuagcc,
(SEQ ID NO: 119)
AAGguaaaaa,
(SEQ ID NO: 120)
AAGguaaaac,
(SEQ ID NO: 121)
AAGguaaaag,
(SEQ ID NO: 122)
AAGguaaaau,
(SEQ ID NO: 123)
AAGguaaaca,
(SEQ ID NO: 124)
AAGguaaacc,
(SEQ ID NO: 125)
AAGguaaacu,
(SEQ ID NO: 126)
AAGguaaaga,
(SEQ ID NO: 127)
AAGguaaagc,
(SEQ ID NO: 128)
AAGguaaagg,
(SEQ ID NO: 129)
AAGguaaagu,
(SEQ ID NO: 130)
AAGguaaaua,
(SEQ ID NO: 131)
AAGguaaauc,
(SEQ ID NO: 132)
AAGguaaaug,
(SEQ ID NO: 133)
AAGguaaauu,
(SEQ ID NO: 134)
AAGguaacaa,
(SEQ ID NO: 135)
AAGguaacau,
(SEQ ID NO: 136)
AAGguaaccc,
(SEQ ID NO: 137)
AAGguaacua,
(SEQ ID NO: 138)
AAGguaacuc,
(SEQ ID NO: 139)
AAGguaacug,
(SEQ ID NO: 140)
AAGguaacuu,
(SEQ ID NO: 141)
AAGguaagaa,
(SEQ ID NO: 142)
AAGguaagac,
(SEQ ID NO: 143)
AAGguaagag,
(SEQ ID NO: 144)
AAGguaagau,
(SEQ ID NO: 145)
AAGguaagca,
(SEQ ID NO: 146)
AAGguaagcc,
(SEQ ID NO: 147)
AAGguaagcg,
(SEQ ID NO: 148)
AAGguaagcu,
(SEQ ID NO: 149)
AAGguaagga,
(SEQ ID NO: 150)
AAGguaaggc,
(SEQ ID NO: 151)
AAGguaaggg,
(SEQ ID NO: 152)
AAGguaaggu,
(SEQ ID NO: 153)
AAGguaagua,
(SEQ ID NO: 154)
AAGguaaguc,
(SEQ ID NO: 155)
AAGguaagug,
(SEQ ID NO: 156)
AAGguaaguu,
(SEQ ID NO: 157)
AAGguaauaa,
(SEQ ID NO: 158)
AAGguaauac,
(SEQ ID NO: 159)
AAGguaauag,
(SEQ ID NO: 160)
AAGguaauau,
(SEQ ID NO: 161)
AAGguaauca,
(SEQ ID NO: 162)
AAGguaaucc,
(SEQ ID NO: 163)
AAGguaaucu,
(SEQ ID NO: 164)
AAGguaauga,
(SEQ ID NO: 165)
AAGguaaugc,
(SEQ ID NO: 166)
AAGguaaugg,
(SEQ ID NO: 167)
AAGguaaugu,
(SEQ ID NO: 168)
AAGguaauua,
(SEQ ID NO: 169)
AAGguaauuc,
(SEQ ID NO: 170)
AAGguaauug,
(SEQ ID NO: 171)
AAGguaauuu,
(SEQ ID NO: 172)
AAGguacaaa,
(SEQ ID NO: 173)
AAGguacaag,
(SEQ ID NO: 174)
AAGguacaau,
(SEQ ID NO: 175)
AAGguacacc,
(SEQ ID NO: 176)
AAGguacacu,
(SEQ ID NO: 177)
AAGguacagg,
(SEQ ID NO: 178)
AAGguacagu,
(SEQ ID NO: 179)
AAGguacaua,
(SEQ ID NO: 180)
AAGguacaug,
(SEQ ID NO: 181)
AAGguacauu,
(SEQ ID NO: 182)
AAGguaccaa,
(SEQ ID NO: 183)
AAGguaccag,
(SEQ ID NO: 184)
AAGguaccca,
(SEQ ID NO: 185)
AAGguacccu,
(SEQ ID NO: 186)
AAGguaccuc,
(SEQ ID NO: 187)
AAGguaccug,
(SEQ ID NO: 188)
AAGguaccuu,
(SEQ ID NO: 189)
AAGguacgaa,
(SEQ ID NO: 190)
AAGguacggg,
(SEQ ID NO: 191)
AAGguacggu,
(SEQ ID NO: 192)
AAGguacguc,
(SEQ ID NO: 193)
AAGguacguu,
(SEQ ID NO: 194)
AAGguacuaa,
(SEQ ID NO: 195)
AAGguacuau,
(SEQ ID NO: 196)
AAGguacucu,
(SEQ ID NO: 197)
AAGguacuga,
(SEQ ID NO: 198)
AAGguacugc,
(SEQ ID NO: 199)
AAGguacugu,
(SEQ ID NO: 200)
AAGguacuuc,
(SEQ ID NO: 201)
AAGguacuug,
(SEQ ID NO: 202)
AAGguacuuu,
(SEQ ID NO: 203)
AAGguagaaa,
(SEQ ID NO: 204)
AAGguagaac,
(SEQ ID NO: 205)
AAGguagaca,
(SEQ ID NO: 206)
AAGguagacc,
(SEQ ID NO: 207)
AAGguagacu,
(SEQ ID NO: 208)
AAGguagagu,
(SEQ ID NO: 209)
AAGguagaua,
(SEQ ID NO: 210)
AAGguagcaa,
(SEQ ID NO: 211)
AAGguagcag,
(SEQ ID NO: 212)
AAGguagcca,
(SEQ ID NO: 213)
AAGguagccu,
(SEQ ID NO: 214)
AAGguagcua,
(SEQ ID NO: 215)
AAGguagcug,
(SEQ ID NO: 216)
AAGguagcuu,
(SEQ ID NO: 217)
AAGguaggaa,
(SEQ ID NO: 218)
AAGguaggag,
(SEQ ID NO: 219)
AAGguaggau,
(SEQ ID NO: 220)
AAGguaggca,
(SEQ ID NO: 221)
AAGguaggcc,
(SEQ ID NO: 222)
AAGguaggcu,
(SEQ ID NO: 223)
AAGguaggga,
(SEQ ID NO: 224)
AAGguagggc,
(SEQ ID NO: 225)
AAGguagggg,
(SEQ ID NO: 226)
AAGguagggu,
(SEQ ID NO: 227)
AAGguaggua,
(SEQ ID NO: 228)
AAGguagguc,
(SEQ ID NO: 229)
AAGguaggug,
(SEQ ID NO: 230)
AAGguagguu,
(SEQ ID NO: 231)
AAGguaguaa,
(SEQ ID NO: 232)
AAGguaguag,
(SEQ ID NO: 233)
AAGguagucu,
(SEQ ID NO: 234)
AAGguagugc,
(SEQ ID NO: 235)
AAGguagugg,
(SEQ ID NO: 236)
AAGguaguuc,
(SEQ ID NO: 237)
AAGguaguuu,
(SEQ ID NO: 238)
AAGguauaaa,
(SEQ ID NO: 239)
AAGguauaau,
(SEQ ID NO: 240)
AAGguauaca,
(SEQ ID NO: 241)
AAGguauacu,
(SEQ ID NO: 242)
AAGguauaua,
(SEQ ID NO: 243)
AAGguauauc,
(SEQ ID NO: 244)
AAGguauaug,
(SEQ ID NO: 245)
AAGguauauu,
(SEQ ID NO: 246)
AAGguaucac,
(SEQ ID NO: 247)
AAGguaucag,
(SEQ ID NO: 248)
AAGguauccc,
(SEQ ID NO: 249)
AAGguauccu,
(SEQ ID NO: 250)
AAGguaucuc,
(SEQ ID NO: 251)
AAGguaucug,
(SEQ ID NO: 252)
AAGguaucuu,
(SEQ ID NO: 253)
AAGguaugaa,
(SEQ ID NO: 254)
AAGguaugac,
(SEQ ID NO: 255)
AAGguaugag,
(SEQ ID NO: 256)
AAGguaugau,
(SEQ ID NO: 257)
AAGguaugca,
(SEQ ID NO: 258)
AAGguaugcc,
(SEQ ID NO: 259)
AAGguaugcu,
(SEQ ID NO: 260)
AAGguaugga,
(SEQ ID NO: 261)
AAGguauggc,
(SEQ ID NO: 262)
AAGguauggg,
(SEQ ID NO: 263)
AAGguaugua,
(SEQ ID NO: 264)
AAGguauguc,
(SEQ ID NO: 265)
AAGguaugug,
(SEQ ID NO: 266)
AAGguauguu,
(SEQ ID NO: 267)
AAGguauuaa,
(SEQ ID NO: 268)
AAGguauuac,
(SEQ ID NO: 269)
AAGguauuag,
(SEQ ID NO: 270)
AAGguauuau,
(SEQ ID NO: 271)
AAGguauucc,
(SEQ ID NO: 272)
AAGguauuga,
(SEQ ID NO: 273)
AAGguauugu,
(SEQ ID NO: 274)
AAGguauuua,
(SEQ ID NO: 275)
AAGguauuuc,
(SEQ ID NO: 276)
AAGguauuug,
(SEQ ID NO: 277)
AAGguauuuu,
(SEQ ID NO: 278)
AAGgucaaau,
(SEQ ID NO: 279)
AAGgucaaga,
(SEQ ID NO: 280)
AAGgucaagu,
(SEQ ID NO: 281)
AAGgucacag,
(SEQ ID NO: 282)
AAGgucagaa,
(SEQ ID NO: 283)
AAGgucagac,
(SEQ ID NO: 284)
AAGgucagag,
(SEQ ID NO: 285)
AAGgucagca,
(SEQ ID NO: 286)
AAGgucagcc,
(SEQ ID NO: 287)
AAGgucagcg,
(SEQ ID NO: 288)
AAGgucagcu,
(SEQ ID NO: 289)
AAGgucagga,
(SEQ ID NO: 290)
AAGgucaggc,
(SEQ ID NO: 291)
AAGgucaggg,
(SEQ ID NO: 292)
AAGgucaggu,
(SEQ ID NO: 293)
AAGgucagua,
(SEQ ID NO: 294)
AAGgucaguc,
(SEQ ID NO: 295)
AAGgucagug,
(SEQ ID NO: 296)
AAGgucaguu,
(SEQ ID NO: 297)
AAGgucauag,
(SEQ ID NO: 298)
AAGgucaucu,
(SEQ ID NO: 299)
AAGguccaca,
(SEQ ID NO: 300)
AAGguccaga,
(SEQ ID NO: 301)
AAGguccaua,
(SEQ ID NO: 302)
AAGgucccag,
(SEQ ID NO: 303)
AAGgucccuc,
(SEQ ID NO: 304)
AAGguccuuc,
(SEQ ID NO: 305)
AAGgucgagg,
(SEQ ID NO: 306)
AAGgucuaau,
(SEQ ID NO: 307)
AAGgucuacc,
(SEQ ID NO: 308)
AAGgucuaua,
(SEQ ID NO: 309)
AAGgucuccu,
(SEQ ID NO: 310)
AAGgucucug,
(SEQ ID NO: 311)
AAGgucucuu,
(SEQ ID NO: 312)
AAGgucugaa,
(SEQ ID NO: 313)
AAGgucugag,
(SEQ ID NO: 314)
AAGgucugga,
(SEQ ID NO: 315)
AAGgucuggg,
(SEQ ID NO: 316)
AAGgucugua,
(SEQ ID NO: 317)
AAGgucuguu,
(SEQ ID NO: 318)
AAGgucuucu,
(SEQ ID NO: 319)
AAGgucuuuu,
(SEQ ID NO: 320)
AAGgugaaac,
(SEQ ID NO: 321)
AAGgugaaag,
(SEQ ID NO: 322)
AAGgugaaau,
(SEQ ID NO: 323)
AAGgugaacu,
(SEQ ID NO: 324)
AAGgugaagc,
(SEQ ID NO: 325)
AAGgugaagg,
(SEQ ID NO: 326)
AAGgugaagu,
(SEQ ID NO: 327)
AAGgugaaua,
(SEQ ID NO: 328)
AAGgugaaug,
(SEQ ID NO: 329)
AAGgugaauu,
(SEQ ID NO: 330)
AAGgugacaa,
(SEQ ID NO: 331)
AAGgugacag,
(SEQ ID NO: 332)
AAGgugacau,
(SEQ ID NO: 333)
AAGgugacug,
(SEQ ID NO: 334)
AAGgugacuu,
(SEQ ID NO: 335)
AAGgugagaa,
(SEQ ID NO: 336)
AAGgugagac,
(SEQ ID NO: 337)
AAGgugagag,
(SEQ ID NO: 338)
AAGgugagau,
(SEQ ID NO: 339)
AAGgugagca,
(SEQ ID NO: 340)
AAGgugagcc,
(SEQ ID NO: 341)
AAGgugagcg,
(SEQ ID NO: 342)
AAGgugagcu,
(SEQ ID NO: 343)
AAGgugagga,
(SEQ ID NO: 344)
AAGgugaggc,
(SEQ ID NO: 345)
AAGgugaggg,
(SEQ ID NO: 346)
AAGgugaggu,
(SEQ ID NO: 347)
AAGgugagua,
(SEQ ID NO: 348)
AAGgugaguc,
(SEQ ID NO: 349)
AAGgugagug,
(SEQ ID NO: 350)
AAGgugaguu,
(SEQ ID NO: 351)
AAGgugauaa,
(SEQ ID NO: 352)
AAGgugauca,
(SEQ ID NO: 353)
AAGgugaucc,
(SEQ ID NO: 354)
AAGgugauga,
(SEQ ID NO: 355)
AAGgugaugc,
(SEQ ID NO: 356)
AAGgugaugu,
(SEQ ID NO: 357)
AAGgugauua,
(SEQ ID NO: 358)
AAGgugauug,
(SEQ ID NO: 359)
AAGgugauuu,
(SEQ ID NO: 360)
AAGgugcaca,
(SEQ ID NO: 361)
AAGgugcauc,
(SEQ ID NO: 362)
AAGgugcccu,
(SEQ ID NO: 363)
AAGgugccug,
(SEQ ID NO: 364)
AAGgugcgug,
(SEQ ID NO: 365)
AAGgugcguu,
(SEQ ID NO: 366)
AAGgugcucc,
(SEQ ID NO: 367)
AAGgugcuga,
(SEQ ID NO: 368)
AAGgugcugc,
(SEQ ID NO: 369)
AAGgugcugg,
(SEQ ID NO: 370)
AAGgugcuua,
(SEQ ID NO: 371)
AAGgugcuuu,
(SEQ ID NO: 372)
AAGguggaua,
(SEQ ID NO: 373)
AAGguggcua,
(SEQ ID NO: 374)
AAGguggcug,
(SEQ ID NO: 375)
AAGguggcuu,
(SEQ ID NO: 376)
AAGgugggaa,
(SEQ ID NO: 377)
AAGgugggag,
(SEQ ID NO: 378)
AAGgugggau,
(SEQ ID NO: 379)
AAGgugggca,
(SEQ ID NO: 380)
AAGgugggcc,
(SEQ ID NO: 381)
AAGgugggcg,
(SEQ ID NO: 382)
AAGgugggga,
(SEQ ID NO: 383)
AAGguggggu,
(SEQ ID NO: 384)
AAGgugggua,
(SEQ ID NO: 385)
AAGgugggug,
(SEQ ID NO: 386)
AAGguggguu,
(SEQ ID NO: 387)
AAGgugguaa,
(SEQ ID NO: 388)
AAGgugguac,
(SEQ ID NO: 389)
AAGgugguau,
(SEQ ID NO: 390)
AAGguggugg,
(SEQ ID NO: 391)
AAGgugguua,
(SEQ ID NO: 392)
AAGgugguuc,
(SEQ ID NO: 393)
AAGgugguuu,
(SEQ ID NO: 394)
AAGguguaag,
(SEQ ID NO: 395)
AAGgugucaa,
(SEQ ID NO: 396)
AAGgugucag,
(SEQ ID NO: 397)
AAGgugucug,
(SEQ ID NO: 398)
AAGgugugaa,
(SEQ ID NO: 399)
AAGgugugag,
(SEQ ID NO: 400)
AAGgugugca,
(SEQ ID NO: 401)
AAGgugugga,
(SEQ ID NO: 402)
AAGguguggu,
(SEQ ID NO: 403)
AAGgugugua,
(SEQ ID NO: 404)
AAGguguguc,
(SEQ ID NO: 405)
AAGgugugug,
(SEQ ID NO: 406)
AAGguguguu,
(SEQ ID NO: 407)
AAGguguucu,
(SEQ ID NO: 408)
AAGguguugc,
(SEQ ID NO: 409)
AAGguguugg,
(SEQ ID NO: 410)
AAGguguuug,
(SEQ ID NO: 411)
AAGguuaaaa,
(SEQ ID NO: 412)
AAGguuaaca,
(SEQ ID NO: 413)
AAGguuaagc,
(SEQ ID NO: 414)
AAGguuaauu,
(SEQ ID NO: 415)
AAGguuacau,
(SEQ ID NO: 416)
AAGguuagaa,
(SEQ ID NO: 417)
AAGguuagau,
(SEQ ID NO: 418)
AAGguuagca,
(SEQ ID NO: 419)
AAGguuagcc,
(SEQ ID NO: 420)
AAGguuagga,
(SEQ ID NO: 421)
AAGguuaggc,
(SEQ ID NO: 422)
AAGguuagua,
(SEQ ID NO: 423)
AAGguuaguc,
(SEQ ID NO: 424)
AAGguuagug,
(SEQ ID NO: 425)
AAGguuaguu,
(SEQ ID NO: 426)
AAGguuauag,
(SEQ ID NO: 427)
AAGguuauga,
(SEQ ID NO: 428)
AAGguucaaa,
(SEQ ID NO: 429)
AAGguucaag,
(SEQ ID NO: 430)
AAGguuccuu,
(SEQ ID NO: 431)
AAGguucggc,
(SEQ ID NO: 432)
AAGguucguu,
(SEQ ID NO: 433)
AAGguucuaa,
(SEQ ID NO: 434)
AAGguucuga,
(SEQ ID NO: 435)
AAGguucuua,
(SEQ ID NO: 436)
AAGguugaau,
(SEQ ID NO: 437)
AAGguugacu,
(SEQ ID NO: 438)
AAGguugagg,
(SEQ ID NO: 439)
AAGguugagu,
(SEQ ID NO: 440)
AAGguugaua,
(SEQ ID NO: 441)
AAGguugcac,
(SEQ ID NO: 442)
AAGguugcug,
(SEQ ID NO: 443)
AAGguuggaa,
(SEQ ID NO: 444)
AAGguuggca,
(SEQ ID NO: 445)
AAGguuggga,
(SEQ ID NO: 446)
AAGguugggg,
(SEQ ID NO: 447)
AAGguuggua,
(SEQ ID NO: 448)
AAGguugguc,
(SEQ ID NO: 449)
AAGguuggug,
(SEQ ID NO: 450)
AAGguugguu,
(SEQ ID NO: 451)
AAGguuguaa,
(SEQ ID NO: 452)
AAGguugucc,
(SEQ ID NO: 453)
AAGguugugc,
(SEQ ID NO: 454)
AAGguuguua,
(SEQ ID NO: 455)
AAGguuuacc,
(SEQ ID NO: 456)
AAGguuuaua,
(SEQ ID NO: 457)
AAGguuuauu,
(SEQ ID NO: 458)
AAGguuuccu,
(SEQ ID NO: 459)
AAGguuucgu,
(SEQ ID NO: 460)
AAGguuugag,
(SEQ ID NO: 461)
AAGguuugca,
(SEQ ID NO: 462)
AAGguuugcc,
(SEQ ID NO: 463)
AAGguuugcu,
(SEQ ID NO: 464)
AAGguuugga,
(SEQ ID NO: 465)
AAGguuuggu,
(SEQ ID NO: 466)
AAGguuugua,
(SEQ ID NO: 467)
AAGguuuguc,
(SEQ ID NO: 468)
AAGguuugug,
(SEQ ID NO: 469)
AAGguuuuaa,
(SEQ ID NO: 470)
AAGguuuuca,
(SEQ ID NO: 471)
AAGguuuucg,
(SEQ ID NO: 472)
AAGguuuugc,
(SEQ ID NO: 473)
AAGguuuugu,
(SEQ ID NO: 474)
AAGguuuuuu,
(SEQ ID NO: 475)
AAUgcaagua,
(SEQ ID NO: 476)
AAUgcaaguc,
(SEQ ID NO: 477)
AAUguaaaca,
(SEQ ID NO: 478)
AAUguaaaua,
(SEQ ID NO: 479)
AAUguaaauc,
(SEQ ID NO: 480)
AAUguaaaug,
(SEQ ID NO: 481)
AAUguaaauu,
(SEQ ID NO: 482)
AAUguaacua,
(SEQ ID NO: 483)
AAUguaagaa,
(SEQ ID NO: 484)
AAUguaagag,
(SEQ ID NO: 485)
AAUguaagau,
(SEQ ID NO: 486)
AAUguaagcc,
(SEQ ID NO: 487)
AAUguaagcu,
(SEQ ID NO: 488)
AAUguaagga,
(SEQ ID NO: 489)
AAUguaagua,
(SEQ ID NO: 490)
AAUguaaguc,
(SEQ ID NO: 491)
AAUguaagug,
(SEQ ID NO: 492)
AAUguaaguu,
(SEQ ID NO: 493)
AAUguaauca,
(SEQ ID NO: 494)
AAUguaauga,
(SEQ ID NO: 495)
AAUguaaugu,
(SEQ ID NO: 496)
AAUguacauc,
(SEQ ID NO: 497)
AAUguacaug,
(SEQ ID NO: 498)
AAUguacgau,
(SEQ ID NO: 499)
AAUguacgua,
(SEQ ID NO: 500)
AAUguacguc,
(SEQ ID NO: 501)
AAUguacgug,
(SEQ ID NO: 502)
AAUguacucu,
(SEQ ID NO: 503)
AAUguaggca,
(SEQ ID NO: 504)
AAUguagguu,
(SEQ ID NO: 505)
AAUguaucua,
(SEQ ID NO: 506)
AAUguaugaa,
(SEQ ID NO: 507)
AAUguaugua,
(SEQ ID NO: 508)
AAUguaugug,
(SEQ ID NO: 509)
AAUguauguu,
(SEQ ID NO: 510)
AAUgucagag,
(SEQ ID NO: 511)
AAUgucagau,
(SEQ ID NO: 512)
AAUgucagcu,
(SEQ ID NO: 513)
AAUgucagua,
(SEQ ID NO: 514)
AAUgucaguc,
(SEQ ID NO: 515)
AAUgucagug,
(SEQ ID NO: 516)
AAUgucaguu,
(SEQ ID NO: 517)
AAUgucggua,
(SEQ ID NO: 518)
AAUgucuguu,
(SEQ ID NO: 519)
AAUgugagaa,
(SEQ ID NO: 520)
AAUgugagca,
(SEQ ID NO: 521)
AAUgugagcc,
(SEQ ID NO: 522)
AAUgugagga,
(SEQ ID NO: 523)
AAUgugagua,
(SEQ ID NO: 524)
AAUgugaguc,
(SEQ ID NO: 525)
AAUgugagug,
(SEQ ID NO: 526)
AAUgugaguu,
(SEQ ID NO: 527)
AAUgugauau,
(SEQ ID NO: 528)
AAUgugcaua,
(SEQ ID NO: 529)
AAUgugcgua,
(SEQ ID NO: 530)
AAUgugcguc,
(SEQ ID NO: 531)
AAUgugggac,
(SEQ ID NO: 532)
AAUguggguc,
(SEQ ID NO: 533)
AAUgugggug,
(SEQ ID NO: 534)
AAUgugguuu,
(SEQ ID NO: 535)
AAUgugugua,
(SEQ ID NO: 536)
AAUguuaagu,
(SEQ ID NO: 537)
AAUguuagaa,
(SEQ ID NO: 538)
AAUguuagau,
(SEQ ID NO: 539)
AAUguuagua,
(SEQ ID NO: 540)
AAUguuggug,
(SEQ ID NO: 541)
ACAgcaagua,
(SEQ ID NO: 542)
ACAguaaaua,
(SEQ ID NO: 543)
ACAguaaaug,
(SEQ ID NO: 544)
ACAguaagaa,
(SEQ ID NO: 545
ACAguaagca)
(SEQ ID NO: 546)
ACAguaagua,
(SEQ ID NO: 547)
ACAguaaguc,
(SEQ ID NO: 548)
ACAguaagug,
(SEQ ID NO: 549)
ACAguaaguu,
(SEQ ID NO: 550)
ACAguacgua,
(SEQ ID NO: 551)
ACAguaggug,
(SEQ ID NO: 552)
ACAguauaac,
(SEQ ID NO: 553)
ACAguaugua,
(SEQ ID NO: 554)
ACAgucaguu,
(SEQ ID NO: 555)
ACAgugagaa,
(SEQ ID NO: 556)
ACAgugagcc,
(SEQ ID NO: 557)
ACAgugagcu,
(SEQ ID NO: 558)
ACAgugagga,
(SEQ ID NO: 559)
ACAgugaggu,
(SEQ ID NO: 560)
ACAgugagua,
(SEQ ID NO: 561)
ACAgugaguc,
(SEQ ID NO: 562)
ACAgugagug,
(SEQ ID NO: 563)
ACAgugaguu,
(SEQ ID NO: 564)
ACAgugggua,
(SEQ ID NO: 565)
ACAguggguu,
(SEQ ID NO: 566)
ACAguguaaa,
(SEQ ID NO: 567)
ACAguuaagc,
(SEQ ID NO: 568)
ACAguuaagu,
(SEQ ID NO: 569)
ACAguuaugu,
(SEQ ID NO: 570)
ACAguugagu,
(SEQ ID NO: 571)
ACAguuguga,
(SEQ ID NO: 572)
ACCguaagua,
(SEQ ID NO: 573)
ACCgugagaa,
(SEQ ID NO: 574)
ACCgugagca,
(SEQ ID NO: 575)
ACCgugaguu,
(SEQ ID NO: 576)
ACCgugggug,
(SEQ ID NO: 577)
ACGguaaaac,
(SEQ ID NO: 578)
ACGguaacua,
(SEQ ID NO: 579)
ACGguaagua,
(SEQ ID NO: 580)
ACGguaagug,
(SEQ ID NO: 581)
ACGguaaguu,
(SEQ ID NO: 582)
ACGguaauua,
(SEQ ID NO: 583)
ACGguaauuu,
(SEQ ID NO: 584)
ACGguacaau,
(SEQ ID NO: 585)
ACGguacagu,
(SEQ ID NO: 586)
ACGguaccag,
(SEQ ID NO: 587)
ACGguacggu,
(SEQ ID NO: 588)
ACGguacgua,
(SEQ ID NO: 589)
ACGguaggaa,
(SEQ ID NO: 590)
ACGguaggag,
(SEQ ID NO: 591)
ACGguaggug,
(SEQ ID NO: 592)
ACGguaguaa,
(SEQ ID NO: 593)
ACGguauaau,
(SEQ ID NO: 594)
ACGguaugac,
(SEQ ID NO: 595)
ACGguaugcg,
(SEQ ID NO: 596)
ACGguaugua,
(SEQ ID NO: 597)
ACGguauguc,
(SEQ ID NO: 598)
ACGgugaaac,
(SEQ ID NO: 599)
ACGgugaagu,
(SEQ ID NO: 600)
ACGgugaauc,
(SEQ ID NO: 601)
ACGgugacag,
(SEQ ID NO: 602)
ACGgugacca,
(SEQ ID NO: 603)
ACGgugagaa,
(SEQ ID NO: 604)
ACGgugagau,
(SEQ ID NO: 605)
ACGgugagcc,
(SEQ ID NO: 606)
ACGgugagua,
(SEQ ID NO: 607)
ACGgugagug,
(SEQ ID NO: 608)
ACGgugaguu,
(SEQ ID NO: 609)
ACGgugcgug,
(SEQ ID NO: 610)
ACGguggcac,
(SEQ ID NO: 611)
ACGguggggc,
(SEQ ID NO: 612)
ACGgugggug,
(SEQ ID NO: 613)
ACGguguagu,
(SEQ ID NO: 614)
ACGgugucac,
(SEQ ID NO: 615)
ACGgugugua,
(SEQ ID NO: 616)
ACGguguguu,
(SEQ ID NO: 617)
ACGguuagug,
(SEQ ID NO: 618)
ACGguuaguu,
(SEQ ID NO: 619)
ACGguucaau,
(SEQ ID NO: 620)
ACUguaaaua,
(SEQ ID NO: 621)
ACUguaagaa,
(SEQ ID NO: 622)
ACUguaagac,
(SEQ ID NO: 623)
ACUguaagca,
(SEQ ID NO: 624)
ACUguaagcu,
(SEQ ID NO: 625)
ACUguaagua,
(SEQ ID NO: 626)
ACUguaaguc,
(SEQ ID NO: 627)
ACUguaaguu,
(SEQ ID NO: 628)
ACUguacguu,
(SEQ ID NO: 629)
ACUguacugc,
(SEQ ID NO: 630)
ACUguaggcu,
(SEQ ID NO: 631)
ACUguaggua,
(SEQ ID NO: 632)
ACUguauauu,
(SEQ ID NO: 633)
ACUguaugaa,
(SEQ ID NO: 634)
ACUguaugcu,
(SEQ ID NO: 635)
ACUguaugug,
(SEQ ID NO: 636)
ACUguauucc,
(SEQ ID NO: 637)
ACUgucagcu,
(SEQ ID NO: 638)
ACUgucagug,
(SEQ ID NO: 639)
ACUgugaacg,
(SEQ ID NO: 640)
ACUgugagca,
(SEQ ID NO: 641)
ACUgugagcg,
(SEQ ID NO: 642)
ACUgugagcu,
(SEQ ID NO: 643)
ACUgugagua,
(SEQ ID NO: 644)
ACUgugaguc,
(SEQ ID NO: 645)
ACUgugagug,
(SEQ ID NO: 646)
ACUgugaguu,
(SEQ ID NO: 647)
ACUgugggua,
(SEQ ID NO: 648)
ACUgugugug,
(SEQ ID NO: 649)
ACUguuaagu,
(SEQ ID NO: 650)
AGAgcaagua,
(SEQ ID NO: 651)
AGAguaaaac,
(SEQ ID NO: 652)
AGAguaaacg,
(SEQ ID NO: 653)
AGAguaaaga,
(SEQ ID NO: 654)
AGAguaaagu,
(SEQ ID NO: 655)
AGAguaaauc,
(SEQ ID NO: 656)
AGAguaaaug,
(SEQ ID NO: 657)
AGAguaacau,
(SEQ ID NO: 658)
AGAguaacua,
(SEQ ID NO: 659)
AGAguaagaa,
(SEQ ID NO: 660)
AGAguaagac,
(SEQ ID NO: 661)
AGAguaagag,
(SEQ ID NO: 662)
AGAguaagau,
(SEQ ID NO: 663)
AGAguaagca,
(SEQ ID NO: 664)
AGAguaagcu,
(SEQ ID NO: 665)
AGAguaagga,
(SEQ ID NO: 666)
AGAguaaggc,
(SEQ ID NO: 667)
AGAguaaggg,
(SEQ ID NO: 668)
AGAguaaggu,
(SEQ ID NO: 669)
AGAguaaguc,
(SEQ ID NO: 670)
AGAguaagug,
(SEQ ID NO: 671)
AGAguaaguu,
(SEQ ID NO: 672)
AGAguaauaa,
(SEQ ID NO: 673)
AGAguaaugu,
(SEQ ID NO: 674)
AGAguaauuc,
(SEQ ID NO: 675)
AGAguaauuu,
(SEQ ID NO: 676)
AGAguacacc,
(SEQ ID NO: 677)
AGAguaccug,
(SEQ ID NO: 678)
AGAguacgug,
(SEQ ID NO: 679)
AGAguacucu,
(SEQ ID NO: 680)
AGAguacuga,
(SEQ ID NO: 681)
AGAguacuuu,
(SEQ ID NO: 682)
AGAguagcug,
(SEQ ID NO: 683)
AGAguaggaa,
(SEQ ID NO: 684)
AGAguaggga,
(SEQ ID NO: 685)
AGAguagggu,
(SEQ ID NO: 686)
AGAguagguc,
(SEQ ID NO: 687)
AGAguaggug,
(SEQ ID NO: 688)
AGAguagguu,
(SEQ ID NO: 689)
AGAguauaua,
(SEQ ID NO: 690)
AGAguauauu,
(SEQ ID NO: 691)
AGAguaugaa,
(SEQ ID NO: 692)
AGAguaugac,
(SEQ ID NO: 693)
AGAguaugau,
(SEQ ID NO: 694)
AGAguauguc,
(SEQ ID NO: 695)
AGAguaugug,
(SEQ ID NO: 696)
AGAguauguu,
(SEQ ID NO: 697)
AGAguauuaa,
(SEQ ID NO: 698)
AGAguauuau,
(SEQ ID NO: 699)
AGAgucagug,
(SEQ ID NO: 700)
AGAgugagac,
(SEQ ID NO: 701)
AGAgugagag,
(SEQ ID NO: 702)
AGAgugagau,
(SEQ ID NO: 703)
AGAgugagca,
(SEQ ID NO: 704)
AGAgugagua,
(SEQ ID NO: 705)
AGAgugaguc,
(SEQ ID NO: 706)
AGAgugagug,
(SEQ ID NO: 707)
AGAgugaguu,
(SEQ ID NO: 708)
AGAgugcguc,
(SEQ ID NO: 709)
AGAgugggga,
(SEQ ID NO: 710)
AGAgugggug,
(SEQ ID NO: 711)
AGAgugugug,
(SEQ ID NO: 712)
AGAguguuuc,
(SEQ ID NO: 713)
AGAguuagua,
(SEQ ID NO: 714)
AGAguugaga,
(SEQ ID NO: 715)
AGAguugagu,
(SEQ ID NO: 716)
AGAguugguu,
(SEQ ID NO: 717)
AGAguuugau,
(SEQ ID NO: 718)
AGCguaagcu,
(SEQ ID NO: 719)
AGCguaagug,
(SEQ ID NO: 720)
AGCgugagcc,
(SEQ ID NO: 721)
AGCgugagug,
(SEQ ID NO: 722)
AGCguuguuc,
(SEQ ID NO: 723)
AGGgcagagu,
(SEQ ID NO: 724)
AGGgcagccu,
(SEQ ID NO: 725)
AGGgcuagua,
(SEQ ID NO: 726)
AGGguaaaga,
(SEQ ID NO: 727)
AGGguaaaua,
(SEQ ID NO: 728)
AGGguaaauc,
(SEQ ID NO: 729)
AGGguaaauu,
(SEQ ID NO: 730)
AGGguaacca,
(SEQ ID NO: 731)
AGGguaacug,
(SEQ ID NO: 732)
AGGguaacuu,
(SEQ ID NO: 733)
AGGguaagaa,
(SEQ ID NO: 734)
AGGguaagag,
(SEQ ID NO: 735)
AGGguaagau,
(SEQ ID NO: 736)
AGGguaagca,
(SEQ ID NO: 737)
AGGguaagga,
(SEQ ID NO: 738)
AGGguaaggc,
(SEQ ID NO: 739)
AGGguaaggg,
(SEQ ID NO: 740)
AGGguaagua,
(SEQ ID NO: 741)
AGGguaaguc,
(SEQ ID NO: 742)
AGGguaagug,
(SEQ ID NO: 743)
AGGguaaguu,
(SEQ ID NO: 744)
AGGguaauac,
(SEQ ID NO: 745)
AGGguaauga,
(SEQ ID NO: 746)
AGGguaauua,
(SEQ ID NO: 747)
AGGguaauuu,
(SEQ ID NO: 748)
AGGguacacc,
(SEQ ID NO: 749)
AGGguacagu,
(SEQ ID NO: 750)
AGGguacggu,
(SEQ ID NO: 751)
AGGguaggac,
(SEQ ID NO: 752)
AGGguaggag,
(SEQ ID NO: 753)
AGGguaggca,
(SEQ ID NO: 754)
AGGguaggcc,
(SEQ ID NO: 755)
AGGguaggga,
(SEQ ID NO: 756)
AGGguagggu,
(SEQ ID NO: 757)
AGGguagguc,
(SEQ ID NO: 758)
AGGguaggug,
(SEQ ID NO: 759)
AGGguagguu,
(SEQ ID NO: 760)
AGGguauaua,
(SEQ ID NO: 761)
AGGguaugac,
(SEQ ID NO: 762)
AGGguaugag,
(SEQ ID NO: 763)
AGGguaugau,
(SEQ ID NO: 764)
AGGguaugca,
(SEQ ID NO: 765)
AGGguaugcu,
(SEQ ID NO: 766)
AGGguauggg,
(SEQ ID NO: 767)
AGGguauggu,
(SEQ ID NO: 768)
AGGguaugua,
(SEQ ID NO: 769)
AGGguauguc,
(SEQ ID NO: 770)
AGGguaugug,
(SEQ ID NO: 771)
AGGguauuac,
(SEQ ID NO: 772)
AGGguauucu,
(SEQ ID NO: 773)
AGGguauuuc,
(SEQ ID NO: 774)
AGGgucagag,
(SEQ ID NO: 775)
AGGgucagca,
(SEQ ID NO: 776)
AGGgucagga,
(SEQ ID NO: 777)
AGGgucaggg,
(SEQ ID NO: 778)
AGGgucagug,
(SEQ ID NO: 779)
AGGgucaguu,
(SEQ ID NO: 780)
AGGguccccu,
(SEQ ID NO: 781)
AGGgucggga,
(SEQ ID NO: 782)
AGGgucugca,
(SEQ ID NO: 783)
AGGgucuguu,
(SEQ ID NO: 784)
AGGgugaaga,
(SEQ ID NO: 785)
AGGgugacua,
(SEQ ID NO: 786)
AGGgugagaa,
(SEQ ID NO: 787)
AGGgugagac,
(SEQ ID NO: 788)
AGGgugagag,
(SEQ ID NO: 789)
AGGgugagca,
(SEQ ID NO: 790)
AGGgugagcc,
(SEQ ID NO: 791)
AGGgugagcu,
(SEQ ID NO: 792)
AGGgugagga,
(SEQ ID NO: 793)
AGGgugaggg,
(SEQ ID NO: 794)
AGGgugaggu,
(SEQ ID NO: 795)
AGGgugagua,
(SEQ ID NO: 796)
AGGgugaguc,
(SEQ ID NO: 797)
AGGgugagug,
(SEQ ID NO: 798)
AGGgugaguu,
(SEQ ID NO: 799)
AGGgugggga,
(SEQ ID NO: 800)
AGGguggggu,
(SEQ ID NO: 801)
AGGgugggua,
(SEQ ID NO: 802)
AGGgugggug,
(SEQ ID NO: 803)
AGGgugugua,
(SEQ ID NO: 804)
AGGgugugug,
(SEQ ID NO: 805)
AGGguuaaug,
(SEQ ID NO: 806)
AGGguuagaa,
(SEQ ID NO: 807)
AGGguuaguu,
(SEQ ID NO: 808)
AGGguuggug,
(SEQ ID NO: 809)
AGGguuugug,
(SEQ ID NO: 810)
AGGguuuguu,
(SEQ ID NO: 811)
AGUguaaaag,
(SEQ ID NO: 812)
AGUguaaaua,
(SEQ ID NO: 813)
AGUguaaauu,
(SEQ ID NO: 814)
AGUguaagaa,
(SEQ ID NO: 815)
AGUguaagag,
(SEQ ID NO: 816)
AGUguaagau,
(SEQ ID NO: 817)
AGUguaagca,
(SEQ ID NO: 818)
AGUguaagcc,
(SEQ ID NO: 819)
AGUguaagua,
(SEQ ID NO: 820)
AGUguaagug,
(SEQ ID NO: 821)
AGUguaaguu,
(SEQ ID NO: 822)
AGUguaauug,
(SEQ ID NO: 823)
AGUguaggac,
(SEQ ID NO: 824)
AGUguagguc,
(SEQ ID NO: 825)
AGUguaugag,
(SEQ ID NO: 826)
AGUguaugua,
(SEQ ID NO: 827)
AGUguauguu,
(SEQ ID NO: 828)
AGUguauugu,
(SEQ ID NO: 829)
AGUguauuua,
(SEQ ID NO: 830)
AGUgucaguc,
(SEQ ID NO: 831)
AGUgugagag,
(SEQ ID NO: 832)
AGUgugagca,
(SEQ ID NO: 833)
AGUgugagcc,
(SEQ ID NO: 834)
AGUgugagcu,
(SEQ ID NO: 835)
AGUgugagua,
(SEQ ID NO: 836)
AGUgugaguc,
(SEQ ID NO: 837)
AGUgugagug,
(SEQ ID NO: 838)
AGUgugaguu,
(SEQ ID NO: 839)
AGUgugggua,
(SEQ ID NO: 840)
AGUgugggug,
(SEQ ID NO: 841)
AGUgugugua,
(SEQ ID NO: 842)
AGUguuccua,
(SEQ ID NO: 843)
AGUguugggg,
(SEQ ID NO: 844)
AGUguuucag,
(SEQ ID NO: 845)
AUAguaaaua,
(SEQ ID NO: 846)
AUAguaagac,
(SEQ ID NO: 847)
AUAguaagau,
(SEQ ID NO: 848)
AUAguaagca,
(SEQ ID NO: 849)
AUAguaagua,
(SEQ ID NO: 850)
AUAguaagug,
(SEQ ID NO: 851)
AUAguaaguu,
(SEQ ID NO: 852)
AUAguaggua,
(SEQ ID NO: 853)
AUAguauguu,
(SEQ ID NO: 854)
AUAgucucac,
(SEQ ID NO: 855)
AUAgugagac,
(SEQ ID NO: 856)
AUAgugagag,
(SEQ ID NO: 857)
AUAgugagau,
(SEQ ID NO: 858)
AUAgugagcc,
(SEQ ID NO: 859)
AUAgugaggc,
(SEQ ID NO: 860)
AUAgugagua,
(SEQ ID NO: 861)
AUAgugaguc,
(SEQ ID NO: 862)
AUAgugagug,
(SEQ ID NO: 863)
AUAgugcguc,
(SEQ ID NO: 864)
AUAgugugua,
(SEQ ID NO: 865)
AUAguucagu,
(SEQ ID NO: 866)
AUCguaagcc,
(SEQ ID NO: 867)
AUCguaaguu,
(SEQ ID NO: 868)
AUCguauucc,
(SEQ ID NO: 869)
AUCgugagua,
(SEQ ID NO: 870)
AUGgcaagcg,
(SEQ ID NO: 871)
AUGgcaagga,
(SEQ ID NO: 872)
AUGgcaaguu,
(SEQ ID NO: 873)
AUGgcaggua,
(SEQ ID NO: 874)
AUGgcaugug,
(SEQ ID NO: 875)
AUGgcgccau,
(SEQ ID NO: 876)
AUGgcuugug,
(SEQ ID NO: 877)
AUGguaaaac,
(SEQ ID NO: 878)
AUGguaaaau,
(SEQ ID NO: 879)
AUGguaaacc,
(SEQ ID NO: 880)
AUGguaaaga,
(SEQ ID NO: 881)
AUGguaaaua,
(SEQ ID NO: 882)
AUGguaaaug,
(SEQ ID NO: 883)
AUGguaaauu,
(SEQ ID NO: 884)
AUGguaacag,
(SEQ ID NO: 885)
AUGguaacau,
(SEQ ID NO: 886)
AUGguaacua,
(SEQ ID NO: 887)
AUGguaacuc,
(SEQ ID NO: 888)
AUGguaacuu,
(SEQ ID NO: 889)
AUGguaagaa,
(SEQ ID NO: 890)
AUGguaagac,
(SEQ ID NO: 891)
AUGguaagag,
(SEQ ID NO: 892)
AUGguaagau,
(SEQ ID NO: 893)
AUGguaagca,
(SEQ ID NO: 894)
AUGguaagcc,
(SEQ ID NO: 895)
AUGguaagcu,
(SEQ ID NO: 896)
AUGguaagga,
(SEQ ID NO: 897)
AUGguaaggg,
(SEQ ID NO: 898)
AUGguaagua,
(SEQ ID NO: 899)
AUGguaaguc,
(SEQ ID NO: 900)
AUGguaagug,
(SEQ ID NO: 901)
AUGguaaguu,
(SEQ ID NO: 902)
AUGguaauaa,
(SEQ ID NO: 903)
AUGguaauau,
(SEQ ID NO: 904)
AUGguaauga,
(SEQ ID NO: 905)
AUGguaaugg,
(SEQ ID NO: 906)
AUGguaauug,
(SEQ ID NO: 907)
AUGguaauuu,
(SEQ ID NO: 908)
AUGguacagc,
(SEQ ID NO: 909)
AUGguacauc,
(SEQ ID NO: 910)
AUGguaccag,
(SEQ ID NO: 911)
AUGguaccug,
(SEQ ID NO: 912)
AUGguacgag,
(SEQ ID NO: 913)
AUGguacggu,
(SEQ ID NO: 914)
AUGguagauc,
(SEQ ID NO: 915)
AUGguagcag,
(SEQ ID NO: 916)
AUGguagcug,
(SEQ ID NO: 917)
AUGguaggaa,
(SEQ ID NO: 918)
AUGguaggau,
(SEQ ID NO: 919)
AUGguaggca,
(SEQ ID NO: 920)
AUGguaggcu,
(SEQ ID NO: 921)
AUGguagggg,
(SEQ ID NO: 922)
AUGguagggu,
(SEQ ID NO: 923)
AUGguaggua,
(SEQ ID NO: 924)
AUGguaggug,
(SEQ ID NO: 925)
AUGguaguuu,
(SEQ ID NO: 926)
AUGguauagu,
(SEQ ID NO: 927)
AUGguauaua,
(SEQ ID NO: 928)
AUGguaucag,
(SEQ ID NO: 929)
AUGguaucuu,
(SEQ ID NO: 930)
AUGguaugau,
(SEQ ID NO: 931)
AUGguaugca,
(SEQ ID NO: 932)
AUGguaugcc,
(SEQ ID NO: 933)
AUGguaugcg,
(SEQ ID NO: 934)
AUGguaugcu,
(SEQ ID NO: 935)
AUGguaugga,
(SEQ ID NO: 936)
AUGguauggc,
(SEQ ID NO: 937)
AUGguaugug,
(SEQ ID NO: 938)
AUGguauguu,
(SEQ ID NO: 939)
AUGguauuau,
(SEQ ID NO: 940)
AUGguauuga,
(SEQ ID NO: 941)
AUGguauuug,
(SEQ ID NO: 942)
AUGgucaggg,
(SEQ ID NO: 943)
AUGgucaguc,
(SEQ ID NO: 944)
AUGgucagug,
(SEQ ID NO: 945)
AUGgucauuu,
(SEQ ID NO: 946)
AUGgugaaaa,
(SEQ ID NO: 947)
AUGgugaaac,
(SEQ ID NO: 948)
AUGgugaaau,
(SEQ ID NO: 949)
AUGgugaacu,
(SEQ ID NO: 950)
AUGgugaaga,
(SEQ ID NO: 951)
AUGgugacgu,
(SEQ ID NO: 952)
AUGgugagaa,
(SEQ ID NO: 953)
AUGgugagac,
(SEQ ID NO: 954)
AUGgugagag,
(SEQ ID NO: 955)
AUGgugagca,
(SEQ ID NO: 956)
AUGgugagcc,
(SEQ ID NO: 957)
AUGgugagcg,
(SEQ ID NO: 958)
AUGgugagcu,
(SEQ ID NO: 959)
AUGgugaggc,
(SEQ ID NO: 960)
AUGgugaggg,
(SEQ ID NO: 961)
AUGgugagua,
(SEQ ID NO: 962)
AUGgugaguc,
(SEQ ID NO: 963)
AUGgugagug,
(SEQ ID NO: 964)
AUGgugaguu,
(SEQ ID NO: 965)
AUGgugauuu,
(SEQ ID NO: 966)
AUGgugcgau,
(SEQ ID NO: 967)
AUGgugcgug,
(SEQ ID NO: 968)
AUGgugggua,
(SEQ ID NO: 969)
AUGgugggug,
(SEQ ID NO: 970)
AUGguggguu,
(SEQ ID NO: 971)
AUGgugguua,
(SEQ ID NO: 972)
AUGguguaag,
(SEQ ID NO: 973)
AUGgugugaa,
(SEQ ID NO: 974)
AUGgugugua,
(SEQ ID NO: 975)
AUGgugugug,
(SEQ ID NO: 976)
AUGguuacuc,
(SEQ ID NO: 977)
AUGguuagca,
(SEQ ID NO: 978)
AUGguuaguc,
(SEQ ID NO: 979)
AUGguuagug,
(SEQ ID NO: 980)
AUGguuaguu,
(SEQ ID NO: 981)
AUGguucagu,
(SEQ ID NO: 982)
AUGguucguc,
(SEQ ID NO: 983)
AUGguuggua,
(SEQ ID NO: 984)
AUGguugguc,
(SEQ ID NO: 985)
AUGguugguu,
(SEQ ID NO: 986)
AUGguuguuu,
(SEQ ID NO: 987)
AUGguuugca,
(SEQ ID NO: 988)
AUGguuugua,
(SEQ ID NO: 989)
AUUgcaagua,
(SEQ ID NO: 990)
AUUguaaaua,
(SEQ ID NO: 991)
AUUguaagau,
(SEQ ID NO: 992)
AUUguaagca,
(SEQ ID NO: 993)
AUUguaagga,
(SEQ ID NO: 994)
AUUguaaggc,
(SEQ ID NO: 995)
AUUguaagua,
(SEQ ID NO: 996)
AUUguaaguc,
(SEQ ID NO: 997)
AUUguaaguu,
(SEQ ID NO: 998)
AUUguaauua,
(SEQ ID NO: 999)
AUUguaauuu,
(SEQ ID NO: 1000)
AUUguacaaa,
(SEQ ID NO: 1001)
AUUguaccuc,
(SEQ ID NO: 1002)
AUUguacgug,
(SEQ ID NO: 1003)
AUUguacuug,
(SEQ ID NO: 1004)
AUUguaggua,
(SEQ ID NO: 1005)
AUUguaugag,
(SEQ ID NO: 1006)
AUUguaugua,
(SEQ ID NO: 1007)
AUUgucuguu,
(SEQ ID NO: 1008)
AUUgugagcu,
(SEQ ID NO: 1009)
AUUgugagua,
(SEQ ID NO: 1010)
AUUgugaguc,
(SEQ ID NO: 1011)
AUUgugaguu,
(SEQ ID NO: 1012)
AUUgugcgug,
(SEQ ID NO: 1013)
AUUgugggug,
(SEQ ID NO: 1014)
AUUguuagug,
(SEQ ID NO: 1015)
CAAguaaaaa,
(SEQ ID NO: 1016)
CAAguaaaua,
(SEQ ID NO: 1017)
CAAguaaauc,
(SEQ ID NO: 1018)
CAAguaaaug,
(SEQ ID NO: 1019)
CAAguaaccc,
(SEQ ID NO: 1020)
CAAguaacua,
(SEQ ID NO: 1021)
CAAguaacug,
(SEQ ID NO: 1022)
CAAguaagaa,
(SEQ ID NO: 1023)
CAAguaagac,
(SEQ ID NO: 1024)
CAAguaagau,
(SEQ ID NO: 1025)
CAAguaaggu,
(SEQ ID NO: 1026)
CAAguaagua,
(SEQ ID NO: 1027)
CAAguaaguc,
(SEQ ID NO: 1028)
CAAguaagug,
(SEQ ID NO: 1029)
CAAguaaguu,
(SEQ ID NO: 1030)
CAAguaaucc,
(SEQ ID NO: 1031)
CAAguaaucu,
(SEQ ID NO: 1032)
CAAguaauua,
(SEQ ID NO: 1033)
CAAguaauuc,
(SEQ ID NO: 1034)
CAAguaauug,
(SEQ ID NO: 1035)
CAAguaauuu,
(SEQ ID NO: 1036)
CAAguacaca,
(SEQ ID NO: 1037)
CAAguacguu,
(SEQ ID NO: 1038)
CAAguacuuu,
(SEQ ID NO: 1039)
CAAguagcug,
(SEQ ID NO: 1040)
CAAguaggau,
(SEQ ID NO: 1041)
CAAguaggua,
(SEQ ID NO: 1042)
CAAguagguc,
(SEQ ID NO: 1043)
CAAguaggug,
(SEQ ID NO: 1044)
CAAguagguu,
(SEQ ID NO: 1045)
CAAguaguuu,
(SEQ ID NO: 1046)
CAAguauaac,
(SEQ ID NO: 1047)
CAAguauaug,
(SEQ ID NO: 1048)
CAAguaucuu,
(SEQ ID NO: 1049)
CAAguaugag,
(SEQ ID NO: 1050)
CAAguaugua,
(SEQ ID NO: 1051)
CAAguauguc,
(SEQ ID NO: 1052)
CAAguaugug,
(SEQ ID NO: 1053)
CAAguauguu,
(SEQ ID NO: 1054)
CAAguauuga,
(SEQ ID NO: 1055)
CAAguauuuc,
(SEQ ID NO: 1056)
CAAgucagac,
(SEQ ID NO: 1057)
CAAgucagua,
(SEQ ID NO: 1058)
CAAgucuaua,
(SEQ ID NO: 1059)
CAAgucugau,
(SEQ ID NO: 1060)
CAAgugacuu,
(SEQ ID NO: 1061)
CAAgugagaa,
(SEQ ID NO: 1062)
CAAgugagac,
(SEQ ID NO: 1063)
CAAgugagca,
(SEQ ID NO: 1064)
CAAgugaggc,
(SEQ ID NO: 1065)
CAAgugaggg,
(SEQ ID NO: 1066)
CAAgugagua,
(SEQ ID NO: 1067)
CAAgugaguc,
(SEQ ID NO: 1068)
CAAgugagug,
(SEQ ID NO: 1069)
CAAgugaucc,
(SEQ ID NO: 1070)
CAAgugaucu,
(SEQ ID NO: 1071)
CAAgugauuc,
(SEQ ID NO: 1072)
CAAgugauug,
(SEQ ID NO: 1073)
CAAgugauuu,
(SEQ ID NO: 1074)
CAAgugccuu,
(SEQ ID NO: 1075)
CAAgugggua,
(SEQ ID NO: 1076)
CAAguggguc,
(SEQ ID NO: 1077)
CAAgugggug,
(SEQ ID NO: 1078)
CAAgugugag,
(SEQ ID NO: 1079)
CAAguuaaaa,
(SEQ ID NO: 1080)
CAAguuaagu,
(SEQ ID NO: 1081)
CAAguuaauc,
(SEQ ID NO: 1082)
CAAguuagaa,
(SEQ ID NO: 1083)
CAAguuaguu,
(SEQ ID NO: 1084)
CAAguucaag,
(SEQ ID NO: 1085)
CAAguuccgu,
(SEQ ID NO: 1086)
CAAguuggua,
(SEQ ID NO: 1087)
CAAguuuagu,
(SEQ ID NO: 1088)
CAAguuucca,
(SEQ ID NO: 1089)
CAAguuuguu,
(SEQ ID NO: 1090)
CACguaagag,
(SEQ ID NO: 1091)
CACguaagca,
(SEQ ID NO: 1092)
CACguaauug,
(SEQ ID NO: 1093)
CACguaggac,
(SEQ ID NO: 1094)
CACguaucga,
(SEQ ID NO: 1095)
CACgucaguu,
(SEQ ID NO: 1096)
CACgugagcu,
(SEQ ID NO: 1097)
CACgugaguc,
(SEQ ID NO: 1098)
CACgugagug,
(SEQ ID NO: 1099)
CAGgcaagaa,
(SEQ ID NO: 1100)
CAGgcaagac,
(SEQ ID NO: 1101)
CAGgcaagag,
(SEQ ID NO: 1102)
CAGgcaagga,
(SEQ ID NO: 1103)
CAGgcaagua,
(SEQ ID NO: 1104)
CAGgcaagug,
(SEQ ID NO: 1105)
CAGgcaaguu,
(SEQ ID NO: 1106)
CAGgcacgca,
(SEQ ID NO: 1107)
CAGgcagagg,
(SEQ ID NO: 1108)
CAGgcaggug,
(SEQ ID NO: 1109)
CAGgcaucau,
(SEQ ID NO: 1110)
CAGgcaugaa,
(SEQ ID NO: 1111)
CAGgcaugag,
(SEQ ID NO: 1112)
CAGgcaugca,
(SEQ ID NO: 1113)
CAGgcaugcg,
(SEQ ID NO: 1114)
CAGgcaugug,
(SEQ ID NO: 1115)
CAGgcgagag,
(SEQ ID NO: 1116)
CAGgcgccug,
(SEQ ID NO: 1117)
CAGgcgugug,
(SEQ ID NO: 1118)
CAGguaaaaa,
(SEQ ID NO: 1119)
CAGguaaaag,
(SEQ ID NO: 1120)
CAGguaaaca,
(SEQ ID NO: 1121)
CAGguaaacc,
(SEQ ID NO: 1122)
CAGguaaaga,
(SEQ ID NO: 1123)
CAGguaaagc,
(SEQ ID NO: 1124)
CAGguaaagu,
(SEQ ID NO: 1125)
CAGguaaaua,
(SEQ ID NO: 1126)
CAGguaaauc,
(SEQ ID NO: 1127)
CAGguaaaug,
(SEQ ID NO: 1128)
CAGguaaauu,
(SEQ ID NO: 1129)
CAGguaacag,
(SEQ ID NO: 1130)
CAGguaacau,
(SEQ ID NO: 1131)
CAGguaacca,
(SEQ ID NO: 1132)
CAGguaaccg,
(SEQ ID NO: 1133)
CAGguaacgu,
(SEQ ID NO: 1134)
CAGguaacua,
(SEQ ID NO: 1135)
CAGguaacuc,
(SEQ ID NO: 1136)
CAGguaacug,
(SEQ ID NO: 1137)
CAGguaacuu,
(SEQ ID NO: 1138)
CAGguaagaa,
(SEQ ID NO: 1139)
CAGguaagac,
(SEQ ID NO: 1140)
CAGguaagag,
(SEQ ID NO: 1141)
CAGguaagau,
(SEQ ID NO: 1142)
CAGguaagcc,
(SEQ ID NO: 1143)
CAGguaagga,
(SEQ ID NO: 1144)
CAGguaaggc,
(SEQ ID NO: 1145)
CAGguaaggg,
(SEQ ID NO: 1146)
CAGguaaggu,
(SEQ ID NO: 1147)
CAGguaagua,
(SEQ ID NO: 1148)
CAGguaagug,
(SEQ ID NO: 1149)
CAGguaaguu,
(SEQ ID NO: 1150)
CAGguaauaa,
(SEQ ID NO: 1151)
CAGguaauau,
(SEQ ID NO: 1152)
CAGguaaucc,
(SEQ ID NO: 1153)
CAGguaaugc,
(SEQ ID NO: 1154)
CAGguaaugg,
(SEQ ID NO: 1155)
CAGguaaugu,
(SEQ ID NO: 1156)
CAGguaauua,
(SEQ ID NO: 1157)
CAGguaauuc,
(SEQ ID NO: 1158)
CAGguaauug,
(SEQ ID NO: 1159)
CAGguaauuu,
(SEQ ID NO: 1160)
CAGguacaaa,
(SEQ ID NO: 1161)
CAGguacaag,
(SEQ ID NO: 1162)
CAGguacaau,
(SEQ ID NO: 1163)
CAGguacaca,
(SEQ ID NO: 1164)
CAGguacacg,
(SEQ ID NO: 1165)
CAGguacaga,
(SEQ ID NO: 1166)
CAGguacagg,
(SEQ ID NO: 1167)
CAGguacagu,
(SEQ ID NO: 1168)
CAGguacaua,
(SEQ ID NO: 1169)
CAGguacaug,
(SEQ ID NO: 1170)
CAGguacauu,
(SEQ ID NO: 1171)
CAGguaccac,
(SEQ ID NO: 1172)
CAGguaccca,
(SEQ ID NO: 1173)
CAGguacccg,
(SEQ ID NO: 1174)
CAGguacccu,
(SEQ ID NO: 1175)
CAGguaccgc,
(SEQ ID NO: 1176)
CAGguaccgg,
(SEQ ID NO: 1177)
CAGguaccuc,
(SEQ ID NO: 1178)
CAGguaccug,
(SEQ ID NO: 1179)
CAGguaccuu,
(SEQ ID NO: 1180)
CAGguacgag,
(SEQ ID NO: 1181)
CAGguacgca,
(SEQ ID NO: 1182)
CAGguacgcc,
(SEQ ID NO: 1183)
CAGguacggu,
(SEQ ID NO: 1184)
CAGguacgua,
(SEQ ID NO: 1185)
CAGguacgug,
(SEQ ID NO: 1186)
CAGguacuaa,
(SEQ ID NO: 1187)
CAGguacuag,
(SEQ ID NO: 1188)
CAGguacuau,
(SEQ ID NO: 1189)
CAGguacucc,
(SEQ ID NO: 1190)
CAGguacucu,
(SEQ ID NO: 1191)
CAGguacuga,
(SEQ ID NO: 1192)
CAGguacugc,
(SEQ ID NO: 1193)
CAGguacugu,
(SEQ ID NO: 1194)
CAGguacuua,
(SEQ ID NO: 1195)
CAGguacuuu,
(SEQ ID NO: 1196)
CAGguagaaa,
(SEQ ID NO: 1197)
CAGguagaac,
(SEQ ID NO: 1198)
CAGguagaag,
(SEQ ID NO: 1199)
CAGguagaca,
(SEQ ID NO: 1200)
CAGguagacc,
(SEQ ID NO: 1201)
CAGguagaga,
(SEQ ID NO: 1202)
CAGguagauu,
(SEQ ID NO: 1203)
CAGguagcaa,
(SEQ ID NO: 1204)
CAGguagcac,
(SEQ ID NO: 1205)
CAGguagcag,
(SEQ ID NO: 1206)
CAGguagcca,
(SEQ ID NO: 1207)
CAGguagcgu,
(SEQ ID NO: 1208)
CAGguagcua,
(SEQ ID NO: 1209)
CAGguagcuc,
(SEQ ID NO: 1210)
CAGguagcug,
(SEQ ID NO: 1211)
CAGguagcuu,
(SEQ ID NO: 1212)
CAGguaggaa,
(SEQ ID NO: 1213)
CAGguaggac,
(SEQ ID NO: 1214)
CAGguaggag,
(SEQ ID NO: 1215)
CAGguaggca,
(SEQ ID NO: 1216)
CAGguaggga,
(SEQ ID NO: 1217)
CAGguagggc,
(SEQ ID NO: 1218)
CAGguagggg,
(SEQ ID NO: 1219)
CAGguagggu,
(SEQ ID NO: 1220)
CAGguaggua,
(SEQ ID NO: 1221)
CAGguagguc,
(SEQ ID NO: 1222)
CAGguaggug,
(SEQ ID NO: 1223)
CAGguagguu,
(SEQ ID NO: 1224)
CAGguaguaa,
(SEQ ID NO: 1225)
CAGguaguau,
(SEQ ID NO: 1226)
CAGguaguca,
(SEQ ID NO: 1227)
CAGguagucc,
(SEQ ID NO: 1228)
CAGguaguga,
(SEQ ID NO: 1229)
CAGguagugu,
(SEQ ID NO: 1230)
CAGguaguuc,
(SEQ ID NO: 1231)
CAGguaguug,
(SEQ ID NO: 1232)
CAGguaguuu,
(SEQ ID NO: 1233)
CAGguauaag,
(SEQ ID NO: 1234)
CAGguauaca,
(SEQ ID NO: 1235)
CAGguauaga,
(SEQ ID NO: 1236)
CAGguauauc,
(SEQ ID NO: 1237)
CAGguauaug,
(SEQ ID NO: 1238)
CAGguauauu,
(SEQ ID NO: 1239)
CAGguaucag,
(SEQ ID NO: 1240)
CAGguaucau,
(SEQ ID NO: 1241)
CAGguauccu,
(SEQ ID NO: 1242)
CAGguaucga,
(SEQ ID NO: 1243)
CAGguaucgc,
(SEQ ID NO: 1244)
CAGguaucua,
(SEQ ID NO: 1245)
CAGguaucug,
(SEQ ID NO: 1246)
CAGguaucuu,
(SEQ ID NO: 1247)
CAGguaugaa,
(SEQ ID NO: 1248)
CAGguaugac,
(SEQ ID NO: 1249)
CAGguaugag,
(SEQ ID NO: 1250)
CAGguaugau,
(SEQ ID NO: 1251)
CAGguaugca,
(SEQ ID NO: 1252)
CAGguaugcc,
(SEQ ID NO: 1253)
CAGguaugcg,
(SEQ ID NO: 1254)
CAGguaugcu,
(SEQ ID NO: 1255)
CAGguaugga,
(SEQ ID NO: 1256)
CAGguauggg,
(SEQ ID NO: 1257)
CAGguauggu,
(SEQ ID NO: 1258)
CAGguaugua,
(SEQ ID NO: 1259)
CAGguauguc,
(SEQ ID NO: 1260)
CAGguaugug,
(SEQ ID NO: 1261)
CAGguauguu,
(SEQ ID NO: 1262)
CAGguauuau,
(SEQ ID NO: 1263)
CAGguauuca,
(SEQ ID NO: 1264)
CAGguauucu,
(SEQ ID NO: 1265)
CAGguauuga,
(SEQ ID NO: 1266)
CAGguauugg,
(SEQ ID NO: 1267)
CAGguauugu,
(SEQ ID NO: 1268)
CAGguauuua,
(SEQ ID NO: 1269)
CAGguauuuc,
(SEQ ID NO: 1270)
CAGguauuug,
(SEQ ID NO: 1271)
CAGguauuuu,
(SEQ ID NO: 1272)
CAGgucaaca,
(SEQ ID NO: 1273)
CAGgucaaug,
(SEQ ID NO: 1274)
CAGgucacgu,
(SEQ ID NO: 1275)
CAGgucagaa,
(SEQ ID NO: 1276)
CAGgucagac,
(SEQ ID NO: 1277)
CAGgucagca,
(SEQ ID NO: 1278)
CAGgucagcc,
(SEQ ID NO: 1279)
CAGgucagcg,
(SEQ ID NO: 1280)
CAGgucagga,
(SEQ ID NO: 1281)
CAGgucagua,
(SEQ ID NO: 1282)
CAGgucaguc,
(SEQ ID NO: 1283)
CAGgucagug,
(SEQ ID NO: 1284)
CAGgucaguu,
(SEQ ID NO: 1285)
CAGgucaucc,
(SEQ ID NO: 1286)
CAGgucaugc,
(SEQ ID NO: 1287)
CAGgucauua,
(SEQ ID NO: 1288)
CAGgucauuu,
(SEQ ID NO: 1289)
CAGguccacc,
(SEQ ID NO: 1290)
CAGguccacu,
(SEQ ID NO: 1291)
CAGguccagu,
(SEQ ID NO: 1292)
CAGguccauc,
(SEQ ID NO: 1293)
CAGguccauu,
(SEQ ID NO: 1294)
CAGgucccag,
(SEQ ID NO: 1295)
CAGgucccug,
(SEQ ID NO: 1296)
CAGguccuga,
(SEQ ID NO: 1297)
CAGguccugc,
(SEQ ID NO: 1298)
CAGguccugg,
(SEQ ID NO: 1299)
CAGgucggcc,
(SEQ ID NO: 1300)
CAGgucggug,
(SEQ ID NO: 1301)
CAGgucguug,
(SEQ ID NO: 1302)
CAGgucucuc,
(SEQ ID NO: 1303)
CAGgucucuu,
(SEQ ID NO: 1304)
CAGgucugag,
(SEQ ID NO: 1305)
CAGgucugcc,
(SEQ ID NO: 1306)
CAGgucugcg,
(SEQ ID NO: 1307)
CAGgucugga,
(SEQ ID NO: 1308)
CAGgucuggu,
(SEQ ID NO: 1309)
CAGgucugua,
(SEQ ID NO: 1310)
CAGgucuguc,
(SEQ ID NO: 1311)
CAGgucugug,
(SEQ ID NO: 1312)
CAGgucuguu,
(SEQ ID NO: 1313)
CAGgucuucc,
(SEQ ID NO: 1314)
CAGgucuuuc,
(SEQ ID NO: 1315)
CAGgugaaag,
(SEQ ID NO: 1316)
CAGgugaaau,
(SEQ ID NO: 1317)
CAGgugaaca,
(SEQ ID NO: 1318)
CAGgugaaga,
(SEQ ID NO: 1319)
CAGgugaagg,
(SEQ ID NO: 1320)
CAGgugaaua,
(SEQ ID NO: 1321)
CAGgugaauc,
(SEQ ID NO: 1322)
CAGgugaauu,
(SEQ ID NO: 1323)
CAGgugacaa,
(SEQ ID NO: 1324)
CAGgugacau,
(SEQ ID NO: 1325)
CAGgugacca,
(SEQ ID NO: 1326)
CAGgugaccc,
(SEQ ID NO: 1327)
CAGgugaccg,
(SEQ ID NO: 1328)
CAGgugaccu,
(SEQ ID NO: 1329)
CAGgugacgg,
(SEQ ID NO: 1330)
CAGgugacua,
(SEQ ID NO: 1331)
CAGgugacuc,
(SEQ ID NO: 1332)
CAGgugacug,
(SEQ ID NO: 1333)
CAGgugagaa,
(SEQ ID NO: 1334)
CAGgugagac,
(SEQ ID NO: 1335)
CAGgugagag,
(SEQ ID NO: 1336)
CAGgugagau,
(SEQ ID NO: 1337)
CAGgugagca,
(SEQ ID NO: 1338)
CAGgugagcc,
(SEQ ID NO: 1339)
CAGgugagcg,
(SEQ ID NO: 1340)
CAGgugagcu,
(SEQ ID NO: 1341)
CAGgugagga,
(SEQ ID NO: 1342)
CAGgugaggc,
(SEQ ID NO: 1343)
CAGgugaggg,
(SEQ ID NO: 1344)
CAGgugaggu,
(SEQ ID NO: 1345)
CAGgugagua,
(SEQ ID NO: 1346)
CAGgugaguc,
(SEQ ID NO: 1347)
CAGgugagug,
(SEQ ID NO: 1348)
CAGgugaguu,
(SEQ ID NO: 1349)
CAGgugauaa,
(SEQ ID NO: 1350)
CAGgugaucc,
(SEQ ID NO: 1351)
CAGgugaucu,
(SEQ ID NO: 1352)
CAGgugaugc,
(SEQ ID NO: 1353)
CAGgugaugg,
(SEQ ID NO: 1354)
CAGgugaugu,
(SEQ ID NO: 1355)
CAGgugauua,
(SEQ ID NO: 1356)
CAGgugauuc,
(SEQ ID NO: 1357)
CAGgugauug,
(SEQ ID NO: 1358)
CAGgugauuu,
(SEQ ID NO: 1359)
CAGgugcaaa,
(SEQ ID NO: 1360)
CAGgugcaag,
(SEQ ID NO: 1361)
CAGgugcaca,
(SEQ ID NO: 1362)
CAGgugcacg,
(SEQ ID NO: 1363)
CAGgugcaga,
(SEQ ID NO: 1364)
CAGgugcagg,
(SEQ ID NO: 1365)
CAGgugcaua,
(SEQ ID NO: 1366)
CAGgugcauc,
(SEQ ID NO: 1367)
CAGgugcaug,
(SEQ ID NO: 1368)
CAGgugccaa,
(SEQ ID NO: 1369)
CAGgugccca,
(SEQ ID NO: 1370)
CAGgugcccc,
(SEQ ID NO: 1371)
CAGgugcccg,
(SEQ ID NO: 1372)
CAGgugccua,
(SEQ ID NO: 1373)
CAGgugccug,
(SEQ ID NO: 1374)
CAGgugcgaa,
(SEQ ID NO: 1375)
CAGgugcgca,
(SEQ ID NO: 1376)
CAGgugcgcc,
(SEQ ID NO: 1377)
CAGgugcgcg,
(SEQ ID NO: 1378)
CAGgugcgga,
(SEQ ID NO: 1379)
CAGgugcggu,
(SEQ ID NO: 1380)
CAGgugcgua,
(SEQ ID NO: 1381)
CAGgugcguc,
(SEQ ID NO: 1382)
CAGgugcgug,
(SEQ ID NO: 1383)
CAGgugcuag,
(SEQ ID NO: 1384)
CAGgugcuau,
(SEQ ID NO: 1385)
CAGgugcuca,
(SEQ ID NO: 1386)
CAGgugcucc,
(SEQ ID NO: 1387)
CAGgugcucg,
(SEQ ID NO: 1388)
CAGgugcugc,
(SEQ ID NO: 1389)
CAGgugcugg,
(SEQ ID NO: 1390)
CAGgugcuua,
(SEQ ID NO: 1391)
CAGgugcuuc,
(SEQ ID NO: 1392)
CAGgugcuug,
(SEQ ID NO: 1393)
CAGguggaac,
(SEQ ID NO: 1394)
CAGguggaag,
(SEQ ID NO: 1395)
CAGguggaau,
(SEQ ID NO: 1396)
CAGguggaga,
(SEQ ID NO: 1397)
CAGguggagu,
(SEQ ID NO: 1398)
CAGguggauu,
(SEQ ID NO: 1399)
CAGguggcca,
(SEQ ID NO: 1400)
CAGguggcuc,
(SEQ ID NO: 1401)
CAGguggcug,
(SEQ ID NO: 1402)
CAGgugggaa,
(SEQ ID NO: 1403)
CAGgugggac,
(SEQ ID NO: 1404)
CAGgugggag,
(SEQ ID NO: 1405)
CAGgugggau,
(SEQ ID NO: 1406)
CAGgugggca,
(SEQ ID NO: 1407)
CAGgugggcc,
(SEQ ID NO: 1408)
CAGgugggcu,
(SEQ ID NO: 1409)
CAGgugggga,
(SEQ ID NO: 1410)
CAGguggggc,
(SEQ ID NO: 1411)
CAGguggggg,
(SEQ ID NO: 1412)
CAGguggggu,
(SEQ ID NO: 1413)
CAGgugggua,
(SEQ ID NO: 1414)
CAGguggguc,
(SEQ ID NO: 1415)
CAGgugggug,
(SEQ ID NO: 1416)
CAGguggguu,
(SEQ ID NO: 1417)
CAGguggucu,
(SEQ ID NO: 1418)
CAGguggugg,
(SEQ ID NO: 1419)
CAGgugguug,
(SEQ ID NO: 1420)
CAGguguaca,
(SEQ ID NO: 1421)
CAGguguagg,
(SEQ ID NO: 1422)
CAGguguauc,
(SEQ ID NO: 1423)
CAGgugucac,
(SEQ ID NO: 1424)
CAGgugucag,
(SEQ ID NO: 1425)
CAGgugucca,
(SEQ ID NO: 1426)
CAGguguccu,
(SEQ ID NO: 1427)
CAGgugucua,
(SEQ ID NO: 1428)
CAGgugucuc,
(SEQ ID NO: 1429)
CAGgugucug,
(SEQ ID NO: 1430)
CAGgugugaa,
(SEQ ID NO: 1431)
CAGgugugac,
(SEQ ID NO: 1432)
CAGgugugag,
(SEQ ID NO: 1433)
CAGgugugau,
(SEQ ID NO: 1434)
CAGgugugca,
(SEQ ID NO: 1435)
CAGgugugcc,
(SEQ ID NO: 1436)
CAGgugugcg,
(SEQ ID NO: 1437)
CAGgugugcu,
(SEQ ID NO: 1438)
CAGgugugga,
(SEQ ID NO: 1439)
CAGguguggc,
(SEQ ID NO: 1440)
CAGgugugua,
(SEQ ID NO: 1441)
CAGguguguc,
(SEQ ID NO: 1442)
CAGgugugug,
(SEQ ID NO: 1443)
CAGguguguu,
(SEQ ID NO: 1444)
CAGguguuua,
(SEQ ID NO: 1445)
CAGguuaaaa,
(SEQ ID NO: 1446)
CAGguuaaua,
(SEQ ID NO: 1447)
CAGguuaauc,
(SEQ ID NO: 1448)
CAGguuaccu,
(SEQ ID NO: 1449)
CAGguuagaa,
(SEQ ID NO: 1450)
CAGguuagag,
(SEQ ID NO: 1451)
CAGguuagau,
(SEQ ID NO: 1452)
CAGguuagcc,
(SEQ ID NO: 1453)
CAGguuaggg,
(SEQ ID NO: 1454)
CAGguuaggu,
(SEQ ID NO: 1455)
CAGguuagua,
(SEQ ID NO: 1456)
CAGguuaguc,
(SEQ ID NO: 1457)
CAGguuagug,
(SEQ ID NO: 1458)
CAGguuaguu,
(SEQ ID NO: 1459)
CAGguuauca,
(SEQ ID NO: 1460)
CAGguuaugu,
(SEQ ID NO: 1461)
CAGguuauua,
(SEQ ID NO: 1462)
CAGguuauug,
(SEQ ID NO: 1463)
CAGguucaaa,
(SEQ ID NO: 1464)
CAGguucaac,
(SEQ ID NO: 1465)
CAGguucaag,
(SEQ ID NO: 1466)
CAGguucaca,
(SEQ ID NO: 1467)
CAGguucacg,
(SEQ ID NO: 1468)
CAGguucagg,
(SEQ ID NO: 1469)
CAGguucaug,
(SEQ ID NO: 1470)
CAGguuccag,
(SEQ ID NO: 1471)
CAGguuccca,
(SEQ ID NO: 1472)
CAGguucccg,
(SEQ ID NO: 1473)
CAGguucgaa,
(SEQ ID NO: 1474)
CAGguucgag,
(SEQ ID NO: 1475)
CAGguucuau,
(SEQ ID NO: 1476)
CAGguucugc,
(SEQ ID NO: 1477)
CAGguucuua,
(SEQ ID NO: 1478)
CAGguucuuc,
(SEQ ID NO: 1479)
CAGguucuuu,
(SEQ ID NO: 1480)
CAGguugaac,
(SEQ ID NO: 1481)
CAGguugaag,
(SEQ ID NO: 1482)
CAGguugagu,
(SEQ ID NO: 1483)
CAGguugaua,
(SEQ ID NO: 1484)
CAGguuggag,
(SEQ ID NO: 1485)
CAGguuggca,
(SEQ ID NO: 1486)
CAGguuggcc,
(SEQ ID NO: 1487)
CAGguugguc,
(SEQ ID NO: 1488)
CAGguuggug,
(SEQ ID NO: 1489)
CAGguugguu,
(SEQ ID NO: 1490)
CAGguuguaa,
(SEQ ID NO: 1491)
CAGguuguac,
(SEQ ID NO: 1492)
CAGguuguau,
(SEQ ID NO: 1493)
CAGguuguca,
(SEQ ID NO: 1494)
CAGguuguga,
(SEQ ID NO: 1495)
CAGguuguug,
(SEQ ID NO: 1496)
CAGguuuaag,
(SEQ ID NO: 1497)
CAGguuuacc,
(SEQ ID NO: 1498)
CAGguuuagc,
(SEQ ID NO: 1499)
CAGguuuagu,
(SEQ ID NO: 1500)
CAGguuucuu,
(SEQ ID NO: 1501)
CAGguuugaa,
(SEQ ID NO: 1502)
CAGguuugag,
(SEQ ID NO: 1503)
CAGguuugau,
(SEQ ID NO: 1504)
CAGguuugcc,
(SEQ ID NO: 1505)
CAGguuugcu,
(SEQ ID NO: 1506)
CAGguuuggg,
(SEQ ID NO: 1507)
CAGguuuggu,
(SEQ ID NO: 1508)
CAGguuugua,
(SEQ ID NO: 1509)
CAGguuugug,
(SEQ ID NO: 1510)
CAGguuuguu,
(SEQ ID NO: 1511)
CAGguuuucu,
(SEQ ID NO: 1512)
CAGguuuugg,
(SEQ ID NO: 1513)
CAGguuuuuc,
(SEQ ID NO: 1514)
CAGguuuuuu,
(SEQ ID NO: 1515)
CAUgcagguu,
(SEQ ID NO: 1516)
CAUguaaaac,
(SEQ ID NO: 1517)
CAUguaacua,
(SEQ ID NO: 1518)
CAUguaagaa,
(SEQ ID NO: 1519)
CAUguaagag,
(SEQ ID NO: 1520)
CAUguaagau,
(SEQ ID NO: 1521)
CAUguaagcc,
(SEQ ID NO: 1522)
CAUguaagua,
(SEQ ID NO: 1523)
CAUguaagug,
(SEQ ID NO: 1524)
CAUguaaguu,
(SEQ ID NO: 1525)
CAUguaauua,
(SEQ ID NO: 1526)
CAUguacaua,
(SEQ ID NO: 1527)
CAUguaccac,
(SEQ ID NO: 1528)
CAUguacguu,
(SEQ ID NO: 1529)
CAUguaggua,
(SEQ ID NO: 1530)
CAUguaggug,
(SEQ ID NO: 1531)
CAUguagguu,
(SEQ ID NO: 1532)
CAUguaugaa,
(SEQ ID NO: 1533)
CAUguaugua,
(SEQ ID NO: 1534)
CAUguaugug,
(SEQ ID NO: 1535)
CAUguauguu,
(SEQ ID NO: 1536)
CAUgugagaa,
(SEQ ID NO: 1537)
CAUgugagca,
(SEQ ID NO: 1538)
CAUgugagcu,
(SEQ ID NO: 1539)
CAUgugagua,
(SEQ ID NO: 1540)
CAUgugaguc,
(SEQ ID NO: 1541)
CAUgugagug,
(SEQ ID NO: 1542)
CAUgugaguu,
(SEQ ID NO: 1543)
CAUgugcgua,
(SEQ ID NO: 1544)
CAUgugggaa,
(SEQ ID NO: 1545)
CAUguggguu,
(SEQ ID NO: 1546)
CAUgugugug,
(SEQ ID NO: 1547)
CAUguguguu,
(SEQ ID NO: 1548)
CAUguuaaua,
(SEQ ID NO: 1549)
CAUguuagcc,
(SEQ ID NO: 1550)
CCAguaagau,
(SEQ ID NO: 1551)
CCAguaagca,
(SEQ ID NO: 1552)
CCAguaagcc,
(SEQ ID NO: 1553)
CCAguaagcu,
(SEQ ID NO: 1554)
CCAguaagga,
(SEQ ID NO: 1555)
CCAguaagua,
(SEQ ID NO: 1556)
CCAguaaguc,
(SEQ ID NO: 1557)
CCAguaagug,
(SEQ ID NO: 1558)
CCAguaaguu,
(SEQ ID NO: 1559)
CCAguaauug,
(SEQ ID NO: 1560)
CCAguacggg,
(SEQ ID NO: 1561)
CCAguagguc,
(SEQ ID NO: 1562)
CCAguauugu,
(SEQ ID NO: 1563)
CCAgugaggc,
(SEQ ID NO: 1564)
CCAgugagua,
(SEQ ID NO: 1565)
CCAgugagug,
(SEQ ID NO: 1566)
CCAguggguc,
(SEQ ID NO: 1567)
CCAguuaguu,
(SEQ ID NO: 1568)
CCAguugagu,
(SEQ ID NO: 1569)
CCCguaagau,
(SEQ ID NO: 1570)
CCCguauguc,
(SEQ ID NO: 1571)
CCCguauguu,
(SEQ ID NO: 1572)
CCCguccugc,
(SEQ ID NO: 1573)
CCCgugagug,
(SEQ ID NO: 1574)
CCGguaaaga,
(SEQ ID NO: 1575)
CCGguaagau,
(SEQ ID NO: 1576)
CCGguaagcc,
(SEQ ID NO: 1577)
CCGguaagga,
(SEQ ID NO: 1578)
CCGguaaggc,
(SEQ ID NO: 1579)
CCGguaaugg,
(SEQ ID NO: 1580)
CCGguacagu,
(SEQ ID NO: 1581)
CCGguacuga,
(SEQ ID NO: 1582)
CCGguauucc,
(SEQ ID NO: 1583)
CCGgucagug,
(SEQ ID NO: 1584)
CCGgugaaaa,
(SEQ ID NO: 1585)
CCGgugagaa,
(SEQ ID NO: 1586)
CCGgugaggg,
(SEQ ID NO: 1587)
CCGgugagug,
(SEQ ID NO: 1588)
CCGgugaguu,
(SEQ ID NO: 1589)
CCGgugcgcg,
(SEQ ID NO: 1590)
CCGgugggcg,
(SEQ ID NO: 1591)
CCGguugguc,
(SEQ ID NO: 1592)
CCUguaaaug,
(SEQ ID NO: 1593)
CCUguaaauu,
(SEQ ID NO: 1594)
CCUguaagaa,
(SEQ ID NO: 1595)
CCUguaagac,
(SEQ ID NO: 1596)
CCUguaagag,
(SEQ ID NO: 1597)
CCUguaagca,
(SEQ ID NO: 1598)
CCUguaagcg,
(SEQ ID NO: 1599)
CCUguaagga,
(SEQ ID NO: 1600)
CCUguaaguu,
(SEQ ID NO: 1601)
CCUguaggua,
(SEQ ID NO: 1602)
CCUguaggug,
(SEQ ID NO: 1603)
CCUguaucuu,
(SEQ ID NO: 1604)
CCUguauggu,
(SEQ ID NO: 1605)
CCUguaugug,
(SEQ ID NO: 1606)
CCUgugagaa,
(SEQ ID NO: 1607)
CCUgugagca,
(SEQ ID NO: 1608)
CCUgugaggg,
(SEQ ID NO: 1609)
CCUgugaguc,
(SEQ ID NO: 1610)
CCUgugagug,
(SEQ ID NO: 1611)
CCUgugaguu,
(SEQ ID NO: 1612)
CCUguggcuc,
(SEQ ID NO: 1613)
CCUgugggua,
(SEQ ID NO: 1614)
CCUgugugua,
(SEQ ID NO: 1615)
CCUguuagaa,
(SEQ ID NO: 1616)
CGAguaaggg,
(SEQ ID NO: 1617)
CGAguaaggu,
(SEQ ID NO: 1618)
CGAguagcug,
(SEQ ID NO: 1619)
CGAguaggug,
(SEQ ID NO: 1620)
CGAguagguu,
(SEQ ID NO: 1621)
CGAgugagca,
(SEQ ID NO: 1622)
CGCguaagag,
(SEQ ID NO: 1623)
CGGgcaggca,
(SEQ ID NO: 1624)
CGGguaagcc,
(SEQ ID NO: 1625)
CGGguaagcu,
(SEQ ID NO: 1626)
CGGguaaguu,
(SEQ ID NO: 1627)
CGGguaauuc,
(SEQ ID NO: 1628)
CGGguaauuu,
(SEQ ID NO: 1629)
CGGguacagu,
(SEQ ID NO: 1630)
CGGguacggg,
(SEQ ID NO: 1631)
CGGguaggag,
(SEQ ID NO: 1632)
CGGguaggcc,
(SEQ ID NO: 1633)
CGGguaggug,
(SEQ ID NO: 1634)
CGGguauuua,
(SEQ ID NO: 1635)
CGGgucugag,
(SEQ ID NO: 1636)
CGGgugaccg,
(SEQ ID NO: 1637)
CGGgugacuc,
(SEQ ID NO: 1638)
CGGgugagaa,
(SEQ ID NO: 1639)
CGGgugaggg,
(SEQ ID NO: 1640)
CGGgugaggu,
(SEQ ID NO: 1641)
CGGgugagua,
(SEQ ID NO: 1642)
CGGgugagug,
(SEQ ID NO: 1643)
CGGgugaguu,
(SEQ ID NO: 1644)
CGGgugauuu,
(SEQ ID NO: 1645)
CGGgugccuu,
(SEQ ID NO: 1646)
CGGgugggag,
(SEQ ID NO: 1647)
CGGgugggug,
(SEQ ID NO: 1648)
CGGguggguu,
(SEQ ID NO: 1649)
CGGguguguc,
(SEQ ID NO: 1650)
CGGgugugug,
(SEQ ID NO: 1651)
CGGguguguu,
(SEQ ID NO: 1652)
CGGguucaag,
(SEQ ID NO: 1653)
CGGguucaug,
(SEQ ID NO: 1654)
CGGguuugcu,
(SEQ ID NO: 1655)
CGUguagggu,
(SEQ ID NO: 1656)
CGUguaugca,
(SEQ ID NO: 1657)
CGUguaugua,
(SEQ ID NO: 1658)
CGUgucugua,
(SEQ ID NO: 1659)
CGUgugagug,
(SEQ ID NO: 1660)
CGUguuuucu,
(SEQ ID NO: 1661)
CUAguaaaug,
(SEQ ID NO: 1662)
CUAguaagcg,
(SEQ ID NO: 1663)
CUAguaagcu,
(SEQ ID NO: 1664)
CUAguaagua,
(SEQ ID NO: 1665)
CUAguaaguc,
(SEQ ID NO: 1666)
CUAguaagug,
(SEQ ID NO: 1667)
CUAguaaguu,
(SEQ ID NO: 1668)
CUAguaauuu,
(SEQ ID NO: 1669)
CUAguaggua,
(SEQ ID NO: 1670)
CUAguagguu,
(SEQ ID NO: 1671)
CUAguaugua,
(SEQ ID NO: 1672)
CUAguauguu,
(SEQ ID NO: 1673)
CUAgugagua,
(SEQ ID NO: 1674)
CUCguaagca,
(SEQ ID NO: 1675)
CUCguaagug,
(SEQ ID NO: 1676)
CUCguaaguu,
(SEQ ID NO: 1677)
CUCguaucug,
(SEQ ID NO: 1678)
CUCgucugug,
(SEQ ID NO: 1679)
CUCgugaaua,
(SEQ ID NO: 1680)
CUCgugagua,
(SEQ ID NO: 1681)
CUCgugauua,
(SEQ ID NO: 1682)
CUGguaaaaa,
(SEQ ID NO: 1683)
CUGguaaaau,
(SEQ ID NO: 1684)
CUGguaaacc,
(SEQ ID NO: 1685)
CUGguaaacg,
(SEQ ID NO: 1686)
CUGguaaagc,
(SEQ ID NO: 1687)
CUGguaaaua,
(SEQ ID NO: 1688)
CUGguaaauc,
(SEQ ID NO: 1689)
CUGguaaaug,
(SEQ ID NO: 1690)
CUGguaaauu,
(SEQ ID NO: 1691)
CUGguaacac,
(SEQ ID NO: 1692)
CUGguaacag,
(SEQ ID NO: 1693)
CUGguaaccc,
(SEQ ID NO: 1694)
CUGguaaccg,
(SEQ ID NO: 1695)
CUGguaacug,
(SEQ ID NO: 1696)
CUGguaacuu,
(SEQ ID NO: 1697)
CUGguaagaa,
(SEQ ID NO: 1698)
CUGguaagag,
(SEQ ID NO: 1699)
CUGguaagau,
(SEQ ID NO: 1700)
CUGguaagca,
(SEQ ID NO: 1701)
CUGguaagcc,
(SEQ ID NO: 1702)
CUGguaagcu,
(SEQ ID NO: 1703)
CUGguaagga,
(SEQ ID NO: 1704)
CUGguaaggc,
(SEQ ID NO: 1705)
CUGguaaggg,
(SEQ ID NO: 1706)
CUGguaaggu,
(SEQ ID NO: 1707)
CUGguaagua,
(SEQ ID NO: 1708)
CUGguaagug,
(SEQ ID NO: 1709)
CUGguaaguu,
(SEQ ID NO: 1710)
CUGguaauga,
(SEQ ID NO: 1711)
CUGguaaugc,
(SEQ ID NO: 1712)
CUGguaauuc,
(SEQ ID NO: 1713)
CUGguaauuu,
(SEQ ID NO: 1714)
CUGguacaac,
(SEQ ID NO: 1715)
CUGguacaau,
(SEQ ID NO: 1716)
CUGguacaga,
(SEQ ID NO: 1717)
CUGguacaua,
(SEQ ID NO: 1718)
CUGguacauu,
(SEQ ID NO: 1719)
CUGguaccau,
(SEQ ID NO: 1720)
CUGguacguu,
(SEQ ID NO: 1721)
CUGguacuaa,
(SEQ ID NO: 1722)
CUGguacuug,
(SEQ ID NO: 1723)
CUGguacuuu,
(SEQ ID NO: 1724)
CUGguagaga,
(SEQ ID NO: 1725)
CUGguagaua,
(SEQ ID NO: 1726)
CUGguagcgu,
(SEQ ID NO: 1727)
CUGguaggau,
(SEQ ID NO: 1728)
CUGguaggca,
(SEQ ID NO: 1729)
CUGguaggua,
(SEQ ID NO: 1730)
CUGguagguc,
(SEQ ID NO: 1731)
CUGguaggug,
(SEQ ID NO: 1732)
CUGguaucaa,
(SEQ ID NO: 1733)
CUGguaugau,
(SEQ ID NO: 1734)
CUGguauggc,
(SEQ ID NO: 1735)
CUGguauggu,
(SEQ ID NO: 1736)
CUGguaugua,
(SEQ ID NO: 1737)
CUGguaugug,
(SEQ ID NO: 1738)
CUGguauguu,
(SEQ ID NO: 1739)
CUGguauuga,
(SEQ ID NO: 1740)
CUGguauuuc,
(SEQ ID NO: 1741)
CUGguauuuu,
(SEQ ID NO: 1742)
CUGgucaaca,
(SEQ ID NO: 1743)
CUGgucagag,
(SEQ ID NO: 1744)
CUGgucccgc,
(SEQ ID NO: 1745)
CUGgucggua,
(SEQ ID NO: 1746)
CUGgucuggg,
(SEQ ID NO: 1747)
CUGgugaagu,
(SEQ ID NO: 1748)
CUGgugaaua,
(SEQ ID NO: 1749)
CUGgugaauu,
(SEQ ID NO: 1750)
CUGgugacua,
(SEQ ID NO: 1751)
CUGgugagaa,
(SEQ ID NO: 1752)
CUGgugagac,
(SEQ ID NO: 1753)
CUGgugagca,
(SEQ ID NO: 1754)
CUGgugagcu,
(SEQ ID NO: 1755)
CUGgugagga,
(SEQ ID NO: 1756)
CUGgugaggc,
(SEQ ID NO: 1757)
CUGgugaggg,
(SEQ ID NO: 1758)
CUGgugaggu,
(SEQ ID NO: 1759)
CUGgugagua,
(SEQ ID NO: 1760)
CUGgugaguc,
(SEQ ID NO: 1761)
CUGgugagug,
(SEQ ID NO: 1762)
CUGgugaguu,
(SEQ ID NO: 1763)
CUGgugauua,
(SEQ ID NO: 1764)
CUGgugauuu,
(SEQ ID NO: 1765)
CUGgugcaga,
(SEQ ID NO: 1766)
CUGgugcgcu,
(SEQ ID NO: 1767)
CUGgugcgug,
(SEQ ID NO: 1768)
CUGgugcuga,
(SEQ ID NO: 1769)
CUGgugggag,
(SEQ ID NO: 1770)
CUGgugggga,
(SEQ ID NO: 1771)
CUGgugggua,
(SEQ ID NO: 1772)
CUGguggguc,
(SEQ ID NO: 1773)
CUGgugggug,
(SEQ ID NO: 1774)
CUGguggguu,
(SEQ ID NO: 1775)
CUGgugugaa,
(SEQ ID NO: 1776)
CUGgugugca,
(SEQ ID NO: 1777)
CUGgugugcu,
(SEQ ID NO: 1778)
CUGguguggu,
(SEQ ID NO: 1779)
CUGgugugug,
(SEQ ID NO: 1780)
CUGguguguu,
(SEQ ID NO: 1781)
CUGguuagcu,
(SEQ ID NO: 1782)
CUGguuagug,
(SEQ ID NO: 1783)
CUGguucgug,
(SEQ ID NO: 1784)
CUGguuggcu,
(SEQ ID NO: 1785)
CUGguuguuu,
(SEQ ID NO: 1786)
CUGguuugua,
(SEQ ID NO: 1787)
CUGguuuguc,
(SEQ ID NO: 1788)
CUGguuugug,
(SEQ ID NO: 1789)
CUUguaaaug,
(SEQ ID NO: 1790)
CUUguaagcu,
(SEQ ID NO: 1791)
CUUguaagga,
(SEQ ID NO: 1792)
CUUguaaggc,
(SEQ ID NO: 1793)
CUUguaagua,
(SEQ ID NO: 1794)
CUUguaagug,
(SEQ ID NO: 1795)
CUUguaaguu,
(SEQ ID NO: 1796)
CUUguacguc,
(SEQ ID NO: 1797)
CUUguacgug,
(SEQ ID NO: 1798)
CUUguaggua,
(SEQ ID NO: 1799)
CUUguagugc,
(SEQ ID NO: 1800)
CUUguauagg,
(SEQ ID NO: 1801)
CUUgucagua,
(SEQ ID NO: 1802)
CUUgugagua,
(SEQ ID NO: 1803)
CUUgugaguc,
(SEQ ID NO: 1804)
CUUgugaguu,
(SEQ ID NO: 1805)
CUUguggguu,
(SEQ ID NO: 1806)
CUUgugugua,
(SEQ ID NO: 1807)
CUUguuagug,
(SEQ ID NO: 1808)
CUUguuugag,
(SEQ ID NO: 1809)
GAAguaaaac,
(SEQ ID NO: 1810)
GAAguaaagc,
(SEQ ID NO: 1811)
GAAguaaagu,
(SEQ ID NO: 1812)
GAAguaaaua,
(SEQ ID NO: 1813)
GAAguaaauu,
(SEQ ID NO: 1814)
GAAguaagaa,
(SEQ ID NO: 1815)
GAAguaagcc,
(SEQ ID NO: 1816)
GAAguaagcu,
(SEQ ID NO: 1817)
GAAguaagga,
(SEQ ID NO: 1818)
GAAguaagua,
(SEQ ID NO: 1819)
GAAguaagug,
(SEQ ID NO: 1820)
GAAguaaguu,
(SEQ ID NO: 1821)
GAAguaauau,
(SEQ ID NO: 1822)
GAAguaaugc,
(SEQ ID NO: 1823)
GAAguaauua,
(SEQ ID NO: 1824)
GAAguaauuu,
(SEQ ID NO: 1825)
GAAguaccau,
(SEQ ID NO: 1826)
GAAguacgua,
(SEQ ID NO: 1827)
GAAguacguc,
(SEQ ID NO: 1828)
GAAguaggca,
(SEQ ID NO: 1829)
GAAguagguc,
(SEQ ID NO: 1830)
GAAguauaaa,
(SEQ ID NO: 1831)
GAAguaugcu,
(SEQ ID NO: 1832)
GAAguaugug,
(SEQ ID NO: 1833)
GAAguauguu,
(SEQ ID NO: 1834)
GAAguauuaa,
(SEQ ID NO: 1835)
GAAgucagug,
(SEQ ID NO: 1836)
GAAgugagag,
(SEQ ID NO: 1837)
GAAgugagcg,
(SEQ ID NO: 1838)
GAAgugaggu,
(SEQ ID NO: 1839)
GAAgugaguc,
(SEQ ID NO: 1840)
GAAgugagug,
(SEQ ID NO: 1841)
GAAgugaguu,
(SEQ ID NO: 1842)
GAAgugauaa,
(SEQ ID NO: 1843)
GAAgugauuc,
(SEQ ID NO: 1844)
GAAgugcgug,
(SEQ ID NO: 1845)
GAAguguggg,
(SEQ ID NO: 1846)
GAAguguguc,
(SEQ ID NO: 1847)
GAAguuggug,
(SEQ ID NO: 1848)
GACguaaagu,
(SEQ ID NO: 1849)
GACguaagcu,
(SEQ ID NO: 1850)
GACguaagua,
(SEQ ID NO: 1851)
GACguaaugg,
(SEQ ID NO: 1852)
GACguaugcc,
(SEQ ID NO: 1853)
GACguauguu,
(SEQ ID NO: 1854)
GACgugagcc,
(SEQ ID NO: 1855)
GACgugagug,
(SEQ ID NO: 1856)
GAGgcaaaug,
(SEQ ID NO: 1857)
GAGgcaagag,
(SEQ ID NO: 1858)
GAGgcaagua,
(SEQ ID NO: 1859)
GAGgcaagug,
(SEQ ID NO: 1860)
GAGgcaaguu,
(SEQ ID NO: 1861)
GAGgcacgag,
(SEQ ID NO: 1862)
GAGgcaggga,
(SEQ ID NO: 1863)
GAGgcaugug,
(SEQ ID NO: 1864)
GAGgcgaagg,
(SEQ ID NO: 1865)
GAGguaaaaa,
(SEQ ID NO: 1866)
GAGguaaaac,
(SEQ ID NO: 1867)
GAGguaaaag,
(SEQ ID NO: 1868)
GAGguaaaau,
(SEQ ID NO: 1869)
GAGguaaacc,
(SEQ ID NO: 1870)
GAGguaaaga,
(SEQ ID NO: 1871)
GAGguaaagc,
(SEQ ID NO: 1872)
GAGguaaagu,
(SEQ ID NO: 1873)
GAGguaaaua,
(SEQ ID NO: 1874)
GAGguaaauc,
(SEQ ID NO: 1875)
GAGguaaaug,
(SEQ ID NO: 1876)
GAGguaaauu,
(SEQ ID NO: 1877)
GAGguaacaa,
(SEQ ID NO: 1878)
GAGguaacag,
(SEQ ID NO: 1879)
GAGguaacca,
(SEQ ID NO: 1880)
GAGguaaccu,
(SEQ ID NO: 1881)
GAGguaacuu,
(SEQ ID NO: 1882)
GAGguaagaa,
(SEQ ID NO: 1883)
GAGguaagag,
(SEQ ID NO: 1884)
GAGguaagau,
(SEQ ID NO: 1885)
GAGguaagca,
(SEQ ID NO: 1886)
GAGguaagcc,
(SEQ ID NO: 1887)
GAGguaagcg,
(SEQ ID NO: 1888)
GAGguaagcu,
(SEQ ID NO: 1889)
GAGguaagga,
(SEQ ID NO: 1890)
GAGguaaggc,
(SEQ ID NO: 1891)
GAGguaaggg,
(SEQ ID NO: 1892)
GAGguaaggu,
(SEQ ID NO: 1893)
GAGguaagua,
(SEQ ID NO: 1894)
GAGguaaguc,
(SEQ ID NO: 1895)
GAGguaauaa,
(SEQ ID NO: 1896)
GAGguaauac,
(SEQ ID NO: 1897)
GAGguaauau,
(SEQ ID NO: 1898)
GAGguaauca,
(SEQ ID NO: 1899)
GAGguaaucu,
(SEQ ID NO: 1900)
GAGguaaugg,
(SEQ ID NO: 1901)
GAGguaaugu,
(SEQ ID NO: 1902)
GAGguaauug,
(SEQ ID NO: 1903)
GAGguaauuu,
(SEQ ID NO: 1904)
GAGguacaaa,
(SEQ ID NO: 1905)
GAGguacaac,
(SEQ ID NO: 1906)
GAGguacaga,
(SEQ ID NO: 1907)
GAGguacagc,
(SEQ ID NO: 1908)
GAGguacagu,
(SEQ ID NO: 1909)
GAGguacaua,
(SEQ ID NO: 1910)
GAGguacauu,
(SEQ ID NO: 1911)
GAGguaccag,
(SEQ ID NO: 1912)
GAGguaccga,
(SEQ ID NO: 1913)
GAGguaccug,
(SEQ ID NO: 1914)
GAGguaccuu,
(SEQ ID NO: 1915)
GAGguacuag,
(SEQ ID NO: 1916)
GAGguacuau,
(SEQ ID NO: 1917)
GAGguacucc,
(SEQ ID NO: 1918)
GAGguacugc,
(SEQ ID NO: 1919)
GAGguacugg,
(SEQ ID NO: 1920)
GAGguacugu,
(SEQ ID NO: 1921)
GAGguacuug,
(SEQ ID NO: 1922)
GAGguacuuu,
(SEQ ID NO: 1923)
GAGguagaag,
(SEQ ID NO: 1924)
GAGguagaga,
(SEQ ID NO: 1925)
GAGguagagg,
(SEQ ID NO: 1926)
GAGguagagu,
(SEQ ID NO: 1927)
GAGguagauc,
(SEQ ID NO: 1928)
GAGguagcua,
(SEQ ID NO: 1929)
GAGguagcug,
(SEQ ID NO: 1930)
GAGguaggaa,
(SEQ ID NO: 1931)
GAGguaggag,
(SEQ ID NO: 1932)
GAGguaggca,
(SEQ ID NO: 1933)
GAGguaggcu,
(SEQ ID NO: 1934)
GAGguaggga,
(SEQ ID NO: 1935)
GAGguagggc,
(SEQ ID NO: 1936)
GAGguagggg,
(SEQ ID NO: 1937)
GAGguaggua,
(SEQ ID NO: 1938)
GAGguaggug,
(SEQ ID NO: 1939)
GAGguagguu,
(SEQ ID NO: 1940)
GAGguaguaa,
(SEQ ID NO: 1941)
GAGguaguag,
(SEQ ID NO: 1942)
GAGguaguau,
(SEQ ID NO: 1943)
GAGguagucu,
(SEQ ID NO: 1944)
GAGguagugc,
(SEQ ID NO: 1945)
GAGguagugg,
(SEQ ID NO: 1946)
GAGguaguua,
(SEQ ID NO: 1947)
GAGguaguug,
(SEQ ID NO: 1948)
GAGguauaag,
(SEQ ID NO: 1949)
GAGguauacu,
(SEQ ID NO: 1950)
GAGguauagc,
(SEQ ID NO: 1951)
GAGguauaug,
(SEQ ID NO: 1952)
GAGguauauu,
(SEQ ID NO: 1953)
GAGguaucau,
(SEQ ID NO: 1954)
GAGguaucug,
(SEQ ID NO: 1955)
GAGguaucuu,
(SEQ ID NO: 1956)
GAGguaugaa,
(SEQ ID NO: 1957)
GAGguaugac,
(SEQ ID NO: 1958)
GAGguaugag,
(SEQ ID NO: 1959)
GAGguaugcc,
(SEQ ID NO: 1960)
GAGguaugcg,
(SEQ ID NO: 1961)
GAGguaugcu,
(SEQ ID NO: 1962)
GAGguaugga,
(SEQ ID NO: 1963)
GAGguauggg,
(SEQ ID NO: 1964)
GAGguauggu,
(SEQ ID NO: 1965)
GAGguaugua,
(SEQ ID NO: 1966)
GAGguauguc,
(SEQ ID NO: 1967)
GAGguaugug,
(SEQ ID NO: 1968)
GAGguauguu,
(SEQ ID NO: 1969)
GAGguauucc,
(SEQ ID NO: 1970)
GAGguauuga,
(SEQ ID NO: 1971)
GAGguauugu,
(SEQ ID NO: 1972)
GAGguauuua,
(SEQ ID NO: 1973)
GAGguauuuc,
(SEQ ID NO: 1974)
GAGguauuug,
(SEQ ID NO: 1975)
GAGguauuuu,
(SEQ ID NO: 1976)
GAGgucaaca,
(SEQ ID NO: 1977)
GAGgucaagg,
(SEQ ID NO: 1978)
GAGgucaaug,
(SEQ ID NO: 1979)
GAGgucacug,
(SEQ ID NO: 1980)
GAGgucagaa,
(SEQ ID NO: 1981)
GAGgucagag,
(SEQ ID NO: 1982)
GAGgucagcu,
(SEQ ID NO: 1983)
GAGgucagga,
(SEQ ID NO: 1984)
GAGgucaggc,
(SEQ ID NO: 1985)
GAGgucaggg,
(SEQ ID NO: 1986)
GAGgucaggu,
(SEQ ID NO: 1987)
GAGgucagua,
(SEQ ID NO: 1988)
GAGgucauau,
(SEQ ID NO: 1989)
GAGgucaugu,
(SEQ ID NO: 1990)
GAGgucauuu,
(SEQ ID NO: 1991)
GAGguccaua,
(SEQ ID NO: 1992)
GAGguccauc,
(SEQ ID NO: 1993)
GAGguccggg,
(SEQ ID NO: 1994)
GAGguccggu,
(SEQ ID NO: 1995)
GAGguccuug,
(SEQ ID NO: 1996)
GAGgucgggg,
(SEQ ID NO: 1997)
GAGgucucgu,
(SEQ ID NO: 1998)
GAGgucugag,
(SEQ ID NO: 1999)
GAGgucuggu,
(SEQ ID NO: 2000)
GAGgucuguc,
(SEQ ID NO: 2001)
GAGgucuguu,
(SEQ ID NO: 2002)
GAGgucuuuu,
(SEQ ID NO: 2003)
GAGgugaaaa,
(SEQ ID NO: 2004)
GAGgugaaau,
(SEQ ID NO: 2005)
GAGgugaaca,
(SEQ ID NO: 2006)
GAGgugaagg,
(SEQ ID NO: 2007)
GAGgugaaua,
(SEQ ID NO: 2008)
GAGgugaauu,
(SEQ ID NO: 2009)
GAGgugacau,
(SEQ ID NO: 2010)
GAGgugacca,
(SEQ ID NO: 2011)
GAGgugaccu,
(SEQ ID NO: 2012)
GAGgugacua,
(SEQ ID NO: 2013)
GAGgugacuu,
(SEQ ID NO: 2014)
GAGgugagaa,
(SEQ ID NO: 2015)
GAGgugagac,
(SEQ ID NO: 2016)
GAGgugagag,
(SEQ ID NO: 2017)
GAGgugagau,
(SEQ ID NO: 2018)
GAGgugagca,
(SEQ ID NO: 2019)
GAGgugagcc,
(SEQ ID NO: 2020)
GAGgugagcg,
(SEQ ID NO: 2021)
GAGgugagcu,
(SEQ ID NO: 2022)
GAGgugagga,
(SEQ ID NO: 2023)
GAGgugaggc,
(SEQ ID NO: 2024)
GAGgugaggg,
(SEQ ID NO: 2025)
GAGgugagua,
(SEQ ID NO: 2026)
GAGgugagug,
(SEQ ID NO: 2027)
GAGgugaguu,
(SEQ ID NO: 2028)
GAGgugauau,
(SEQ ID NO: 2029)
GAGgugaucc,
(SEQ ID NO: 2030)
GAGgugaucu,
(SEQ ID NO: 2031)
GAGgugauga,
(SEQ ID NO: 2032)
GAGgugaugg,
(SEQ ID NO: 2033)
GAGgugaugu,
(SEQ ID NO: 2034)
GAGgugauuc,
(SEQ ID NO: 2035)
GAGgugcaca,
(SEQ ID NO: 2036)
GAGgugcaga,
(SEQ ID NO: 2037)
GAGgugcagc,
(SEQ ID NO: 2038)
GAGgugcagg,
(SEQ ID NO: 2039)
GAGgugccag,
(SEQ ID NO: 2040)
GAGgugccca,
(SEQ ID NO: 2041)
GAGgugccuu,
(SEQ ID NO: 2042)
GAGgugcggg,
(SEQ ID NO: 2043)
GAGgugcgug,
(SEQ ID NO: 2044)
GAGgugcucc,
(SEQ ID NO: 2045)
GAGgugcugg,
(SEQ ID NO: 2046)
GAGgugcuua,
(SEQ ID NO: 2047)
GAGgugcuug,
(SEQ ID NO: 2048)
GAGguggaaa,
(SEQ ID NO: 2049)
GAGguggaau,
(SEQ ID NO: 2050)
GAGguggacc,
(SEQ ID NO: 2051)
GAGguggacg,
(SEQ ID NO: 2052)
GAGguggagg,
(SEQ ID NO: 2053)
GAGguggcug,
(SEQ ID NO: 2054)
GAGgugggaa,
(SEQ ID NO: 2055)
GAGgugggag,
(SEQ ID NO: 2056)
GAGgugggau,
(SEQ ID NO: 2057)
GAGgugggca,
(SEQ ID NO: 2058)
GAGgugggcg,
(SEQ ID NO: 2059)
GAGgugggcu,
(SEQ ID NO: 2060)
GAGgugggga,
(SEQ ID NO: 2061)
GAGguggggc,
(SEQ ID NO: 2062)
GAGguggggg,
(SEQ ID NO: 2063)
GAGgugggua,
(SEQ ID NO: 2064)
GAGguggguc,
(SEQ ID NO: 2065)
GAGgugggug,
(SEQ ID NO: 2066)
GAGguggguu,
(SEQ ID NO: 2067)
GAGgugguau,
(SEQ ID NO: 2068)
GAGgugguuc,
(SEQ ID NO: 2069)
GAGgugucau,
(SEQ ID NO: 2070)
GAGgugugag,
(SEQ ID NO: 2071)
GAGgugugau,
(SEQ ID NO: 2072)
GAGgugugca,
(SEQ ID NO: 2073)
GAGgugugcu,
(SEQ ID NO: 2074)
GAGgugugga,
(SEQ ID NO: 2075)
GAGguguggg,
(SEQ ID NO: 2076)
GAGguguggu,
(SEQ ID NO: 2077)
GAGgugugua,
(SEQ ID NO: 2078)
GAGgugugug,
(SEQ ID NO: 2079)
GAGguuaaau,
(SEQ ID NO: 2080)
GAGguuaaga,
(SEQ ID NO: 2081)
GAGguuaaua,
(SEQ ID NO: 2082)
GAGguuaccg,
(SEQ ID NO: 2083)
GAGguuagaa,
(SEQ ID NO: 2084)
GAGguuagac,
(SEQ ID NO: 2085)
GAGguuagag,
(SEQ ID NO: 2086)
GAGguuaggu,
(SEQ ID NO: 2087)
GAGguuagua,
(SEQ ID NO: 2088)
GAGguuaguc,
(SEQ ID NO: 2089)
GAGguuagug,
(SEQ ID NO: 2090)
GAGguuaguu,
(SEQ ID NO: 2091)
GAGguuaugu,
(SEQ ID NO: 2092)
GAGguuauuc,
(SEQ ID NO: 2093)
GAGguucaaa,
(SEQ ID NO: 2094)
GAGguucaua,
(SEQ ID NO: 2095)
GAGguucuga,
(SEQ ID NO: 2096)
GAGguugaag,
(SEQ ID NO: 2097)
GAGguugcag,
(SEQ ID NO: 2098)
GAGguugcug,
(SEQ ID NO: 2099)
GAGguuggaa,
(SEQ ID NO: 2100)
GAGguuggag,
(SEQ ID NO: 2101)
GAGguuggau,
(SEQ ID NO: 2102)
GAGguuggua,
(SEQ ID NO: 2103)
GAGguugguc,
(SEQ ID NO: 2104)
GAGguugguu,
(SEQ ID NO: 2105)
GAGguuguag,
(SEQ ID NO: 2106)
GAGguuucug,
(SEQ ID NO: 2107)
GAGguuugag,
(SEQ ID NO: 2108)
GAGguuugga,
(SEQ ID NO: 2109)
GAGguuuggg,
(SEQ ID NO: 2110)
GAGguuugua,
(SEQ ID NO: 2111)
GAGguuuguu,
(SEQ ID NO: 2112)
GAGguuuuca,
(SEQ ID NO: 2113)
GAGguuuuga,
(SEQ ID NO: 2114)
GAGguuuugg,
(SEQ ID NO: 2115)
GAGguuuuua,
(SEQ ID NO: 2116)
GAGguuuuuc,
(SEQ ID NO: 2117)
GAUguaaaau,
(SEQ ID NO: 2118)
GAUguaagca,
(SEQ ID NO: 2119)
GAUguaagcc,
(SEQ ID NO: 2120)
GAUguaaggu,
(SEQ ID NO: 2121)
GAUguaagua,
(SEQ ID NO: 2122)
GAUguaagug,
(SEQ ID NO: 2123)
GAUguaaguu,
(SEQ ID NO: 2124)
GAUguacauc,
(SEQ ID NO: 2125)
GAUguaggua,
(SEQ ID NO: 2126)
GAUguauggc,
(SEQ ID NO: 2127)
GAUguaugua,
(SEQ ID NO: 2128)
GAUguauguu,
(SEQ ID NO: 2129)
GAUgucagug,
(SEQ ID NO: 2130)
GAUgugagag,
(SEQ ID NO: 2131)
GAUgugagcc,
(SEQ ID NO: 2132)
GAUgugagcu,
(SEQ ID NO: 2133)
GAUgugagga,
(SEQ ID NO: 2134)
GAUgugaguc,
(SEQ ID NO: 2135)
GAUgugagug,
(SEQ ID NO: 2136)
GAUgugaguu,
(SEQ ID NO: 2137)
GAUgugggua,
(SEQ ID NO: 2138)
GAUgugggug,
(SEQ ID NO: 2139)
GAUguguguu,
(SEQ ID NO: 2140)
GAUguuagcu,
(SEQ ID NO: 2141)
GAUguucagu,
(SEQ ID NO: 2142)
GAUguucgug,
(SEQ ID NO: 2143)
GAUguuuguu,
(SEQ ID NO: 2144)
GCAguaaagg,
(SEQ ID NO: 2145)
GCAguaagaa,
(SEQ ID NO: 2146)
GCAguaagga,
(SEQ ID NO: 2147)
GCAguaagua,
(SEQ ID NO: 2148)
GCAguaaguc,
(SEQ ID NO: 2149)
GCAguaaguu,
(SEQ ID NO: 2150)
GCAguagaug,
(SEQ ID NO: 2151)
GCAguaggua,
(SEQ ID NO: 2152)
GCAguaugug,
(SEQ ID NO: 2153)
GCAguauguu,
(SEQ ID NO: 2154)
GCAgucagua,
(SEQ ID NO: 2155)
GCAgucagug,
(SEQ ID NO: 2156)
GCAguccggu,
(SEQ ID NO: 2157)
GCAgugacuu,
(SEQ ID NO: 2158)
GCAgugagcc,
(SEQ ID NO: 2159)
GCAgugagcg,
(SEQ ID NO: 2160)
GCAgugagcu,
(SEQ ID NO: 2161)
GCAgugagua,
(SEQ ID NO: 2162)
GCAgugagug,
(SEQ ID NO: 2163)
GCAgugaguu,
(SEQ ID NO: 2164)
GCAgugggua,
(SEQ ID NO: 2165)
GCAguuaagu,
(SEQ ID NO: 2166)
GCAguugagu,
(SEQ ID NO: 2167)
GCCguaaguc,
(SEQ ID NO: 2168)
GCCgugagua,
(SEQ ID NO: 2169)
GCGguaaagc,
(SEQ ID NO: 2170)
GCGguaaaua,
(SEQ ID NO: 2171)
GCGguaagcu,
(SEQ ID NO: 2172)
GCGguaaggg,
(SEQ ID NO: 2173)
GCGguaagug,
(SEQ ID NO: 2174)
GCGguaauca,
(SEQ ID NO: 2175)
GCGguacgua,
(SEQ ID NO: 2176)
GCGguacuug,
(SEQ ID NO: 2177)
GCGguagggu,
(SEQ ID NO: 2178)
GCGguagugu,
(SEQ ID NO: 2179)
GCGgugagca,
(SEQ ID NO: 2180)
GCGgugagcu,
(SEQ ID NO: 2181)
GCGgugaguu,
(SEQ ID NO: 2182)
GCGguggcuc,
(SEQ ID NO: 2183)
GCGgugugca,
(SEQ ID NO: 2184)
GCGguguguu,
(SEQ ID NO: 2185)
GCGguuaagu,
(SEQ ID NO: 2186)
GCGguuugca,
(SEQ ID NO: 2187)
GCUgcuguaa,
(SEQ ID NO: 2188)
GCUguaaaua,
(SEQ ID NO: 2189)
GCUguaagac,
(SEQ ID NO: 2190)
GCUguaagag,
(SEQ ID NO: 2191)
GCUguaagca,
(SEQ ID NO: 2192)
GCUguaagga,
(SEQ ID NO: 2193)
GCUguaagua,
(SEQ ID NO: 2194)
GCUguaaguc,
(SEQ ID NO: 2195)
GCUguaagug,
(SEQ ID NO: 2196)
GCUguaaguu,
(SEQ ID NO: 2197)
GCUguaggug,
(SEQ ID NO: 2198)
GCUguauggu,
(SEQ ID NO: 2199)
GCUgucagug,
(SEQ ID NO: 2200)
GCUguccuug,
(SEQ ID NO: 2201)
GCUgugagaa,
(SEQ ID NO: 2202)
GCUgugagcc,
(SEQ ID NO: 2203)
GCUgugagga,
(SEQ ID NO: 2204)
GCUgugagua,
(SEQ ID NO: 2205)
GCUgugaguc,
(SEQ ID NO: 2206)
GCUgugagug,
(SEQ ID NO: 2207)
GCUgugaguu,
(SEQ ID NO: 2208)
GCUguggguu,
(SEQ ID NO: 2209)
GGAguaagag,
(SEQ ID NO: 2210)
GGAguaagca,
(SEQ ID NO: 2211)
GGAguaagcc,
(SEQ ID NO: 2212)
GGAguaagcu,
(SEQ ID NO: 2213)
GGAguaagga,
(SEQ ID NO: 2214)
GGAguaagug,
(SEQ ID NO: 2215)
GGAguaaguu,
(SEQ ID NO: 2216)
GGAguaauuu,
(SEQ ID NO: 2217)
GGAguacugu,
(SEQ ID NO: 2218)
GGAguaggaa,
(SEQ ID NO: 2219)
GGAguaggua,
(SEQ ID NO: 2220)
GGAguagguu,
(SEQ ID NO: 2221)
GGAguaguau,
(SEQ ID NO: 2222)
GGAguaugac,
(SEQ ID NO: 2223)
GGAguauggu,
(SEQ ID NO: 2224)
GGAgucaagu,
(SEQ ID NO: 2225)
GGAgugaggg,
(SEQ ID NO: 2226)
GGAgugagua,
(SEQ ID NO: 2227)
GGAgugaguc,
(SEQ ID NO: 2228)
GGAgugagug,
(SEQ ID NO: 2229)
GGAgugaguu,
(SEQ ID NO: 2230)
GGAgugcuuu,
(SEQ ID NO: 2231)
GGAgugggca,
(SEQ ID NO: 2232)
GGAgugggug,
(SEQ ID NO: 2233)
GGAguuaagg,
(SEQ ID NO: 2234)
GGAguugaga,
(SEQ ID NO: 2235)
GGCguaagcc,
(SEQ ID NO: 2236)
GGCguaggua,
(SEQ ID NO: 2237)
GGCguaggug,
(SEQ ID NO: 2238)
GGCgugagcc,
(SEQ ID NO: 2239)
GGCgugaguc,
(SEQ ID NO: 2240)
GGGguaaaca,
(SEQ ID NO: 2241)
GGGguaaacc,
(SEQ ID NO: 2242)
GGGguaaacu,
(SEQ ID NO: 2243)
GGGguaagaa,
(SEQ ID NO: 2244)
GGGguaagag,
(SEQ ID NO: 2245)
GGGguaagau,
(SEQ ID NO: 2246)
GGGguaagca,
(SEQ ID NO: 2247)
GGGguaagcc,
(SEQ ID NO: 2248)
GGGguaagcu,
(SEQ ID NO: 2249)
GGGguaagga,
(SEQ ID NO: 2250)
GGGguaaggg,
(SEQ ID NO: 2251)
GGGguaagua,
(SEQ ID NO: 2252)
GGGguaagug,
(SEQ ID NO: 2253)
GGGguaaguu,
(SEQ ID NO: 2254)
GGGguagaca,
(SEQ ID NO: 2255)
GGGguaggag,
(SEQ ID NO: 2256)
GGGguaggcc,
(SEQ ID NO: 2257)
GGGguaggga,
(SEQ ID NO: 2258)
GGGguaggua,
(SEQ ID NO: 2259)
GGGguaggug,
(SEQ ID NO: 2260)
GGGguagguu,
(SEQ ID NO: 2261)
GGGguagugc,
(SEQ ID NO: 2262)
GGGguaucug,
(SEQ ID NO: 2263)
GGGguaugac,
(SEQ ID NO: 2264)
GGGguaugga,
(SEQ ID NO: 2265)
GGGguaugua,
(SEQ ID NO: 2266)
GGGguauguc,
(SEQ ID NO: 2267)
GGGguaugug,
(SEQ ID NO: 2268)
GGGguauguu,
(SEQ ID NO: 2269)
GGGgucagua,
(SEQ ID NO: 2270)
GGGguccgug,
(SEQ ID NO: 2271)
GGGgucggag,
(SEQ ID NO: 2272)
GGGgucugug,
(SEQ ID NO: 2273)
GGGgugaaca,
(SEQ ID NO: 2274)
GGGgugaaga,
(SEQ ID NO: 2275)
GGGgugagaa,
(SEQ ID NO: 2276)
GGGgugagau,
(SEQ ID NO: 2277)
GGGgugagcc,
(SEQ ID NO: 2278)
GGGgugagcg,
(SEQ ID NO: 2279)
GGGgugagcu,
(SEQ ID NO: 2280)
GGGgugagga,
(SEQ ID NO: 2281)
GGGgugaggc,
(SEQ ID NO: 2282)
GGGgugaggg,
(SEQ ID NO: 2283)
GGGgugaguc,
(SEQ ID NO: 2284)
GGGgugagug,
(SEQ ID NO: 2285)
GGGgugaguu,
(SEQ ID NO: 2286)
GGGgugcgua,
(SEQ ID NO: 2287)
GGGguggggu,
(SEQ ID NO: 2288)
GGGgugggua,
(SEQ ID NO: 2289)
GGGgugggug,
(SEQ ID NO: 2290)
GGGguggguu,
(SEQ ID NO: 2291)
GGGgugugcg,
(SEQ ID NO: 2292)
GGGgugugua,
(SEQ ID NO: 2293)
GGGguguguc,
(SEQ ID NO: 2294)
GGGgugugug,
(SEQ ID NO: 2295)
GGGguuacag,
(SEQ ID NO: 2296)
GGGguuggac,
(SEQ ID NO: 2297)
GGGguuggga,
(SEQ ID NO: 2298)
GGGguuugcc,
(SEQ ID NO: 2299)
GGGguuugua,
(SEQ ID NO: 2300)
GGUguaagaa,
(SEQ ID NO: 2301)
GGUguaagau,
(SEQ ID NO: 2302)
GGUguaagca,
(SEQ ID NO: 2303)
GGUguaagcc,
(SEQ ID NO: 2304)
GGUguaagcg,
(SEQ ID NO: 2305)
GGUguaaguc,
(SEQ ID NO: 2306)
GGUguaagug,
(SEQ ID NO: 2307)
GGUguagguc,
(SEQ ID NO: 2308)
GGUguaggug,
(SEQ ID NO: 2309)
GGUguagguu,
(SEQ ID NO: 2310)
GGUguccgua,
(SEQ ID NO: 2311)
GGUgugagag,
(SEQ ID NO: 2312)
GGUgugagcc,
(SEQ ID NO: 2313)
GGUgugagcu,
(SEQ ID NO: 2314)
GGUgugagua,
(SEQ ID NO: 2315)
GGUgugaguc,
(SEQ ID NO: 2316)
GGUgugcuuc,
(SEQ ID NO: 2317)
GGUguggcug,
(SEQ ID NO: 2318)
GGUgugguga,
(SEQ ID NO: 2319)
GGUgugucug,
(SEQ ID NO: 2320)
GGUguugaaa,
(SEQ ID NO: 2321)
GGUguugcug,
(SEQ ID NO: 2322)
GUAguaagau,
(SEQ ID NO: 2323)
GUAguaagua,
(SEQ ID NO: 2324)
GUAguaagug,
(SEQ ID NO: 2325)
GUAguagcuu,
(SEQ ID NO: 2326)
GUAguaggua,
(SEQ ID NO: 2327)
GUAgucagua,
(SEQ ID NO: 2328)
GUAgugagua,
(SEQ ID NO: 2329)
GUAguggugg,
(SEQ ID NO: 2330)
GUAguuaagu,
(SEQ ID NO: 2331)
GUAguuucug,
(SEQ ID NO: 2332)
GUCguaagug,
(SEQ ID NO: 2333)
GUCgugagug,
(SEQ ID NO: 2334)
GUCgugaguu,
(SEQ ID NO: 2335)
GUGgcaagua,
(SEQ ID NO: 2336)
GUGgcuugua,
(SEQ ID NO: 2337)
GUGguaaaau,
(SEQ ID NO: 2338)
GUGguaaaga,
(SEQ ID NO: 2339)
GUGguaaauu,
(SEQ ID NO: 2340)
GUGguaacau,
(SEQ ID NO: 2341)
GUGguaacua,
(SEQ ID NO: 2342)
GUGguaagaa,
(SEQ ID NO: 2343)
GUGguaagac,
(SEQ ID NO: 2344)
GUGguaagag,
(SEQ ID NO: 2345)
GUGguaagau,
(SEQ ID NO: 2346)
GUGguaagca,
(SEQ ID NO: 2347)
GUGguaagcg,
(SEQ ID NO: 2348)
GUGguaagcu,
(SEQ ID NO: 2349)
GUGguaagga,
(SEQ ID NO: 2350)
GUGguaaggc,
(SEQ ID NO: 2351)
GUGguaagua,
(SEQ ID NO: 2352)
GUGguaaguc,
(SEQ ID NO: 2353)
GUGguaagug,
(SEQ ID NO: 2354)
GUGguaaguu,
(SEQ ID NO: 2355)
GUGguaauga,
(SEQ ID NO: 2356)
GUGguaauuc,
(SEQ ID NO: 2357)
GUGguaauuu,
(SEQ ID NO: 2358)
GUGguacaug,
(SEQ ID NO: 2359)
GUGguacgau,
(SEQ ID NO: 2360)
GUGguacuau,
(SEQ ID NO: 2361)
GUGguacuug,
(SEQ ID NO: 2362)
GUGguagaua,
(SEQ ID NO: 2363)
GUGguagcgc,
(SEQ ID NO: 2364)
GUGguaggga,
(SEQ ID NO: 2365)
GUGguagguc,
(SEQ ID NO: 2366)
GUGguaggug,
(SEQ ID NO: 2367)
GUGguagguu,
(SEQ ID NO: 2368)
GUGguauaaa,
(SEQ ID NO: 2369)
GUGguaucuc,
(SEQ ID NO: 2370)
GUGguaugaa,
(SEQ ID NO: 2371)
GUGguaugau,
(SEQ ID NO: 2372)
GUGguaugca,
(SEQ ID NO: 2373)
GUGguaugua,
(SEQ ID NO: 2374)
GUGguauguu,
(SEQ ID NO: 2375)
GUGguccgug,
(SEQ ID NO: 2376)
GUGgucuggc,
(SEQ ID NO: 2377)
GUGgugaaac,
(SEQ ID NO: 2378)
GUGgugagaa,
(SEQ ID NO: 2379)
GUGgugagau,
(SEQ ID NO: 2380)
GUGgugagca,
(SEQ ID NO: 2381)
GUGgugagcu,
(SEQ ID NO: 2382)
GUGgugagga,
(SEQ ID NO: 2383)
GUGgugaggc,
(SEQ ID NO: 2384)
GUGgugagug,
(SEQ ID NO: 2385)
GUGgugaguu,
(SEQ ID NO: 2386)
GUGgugauua,
(SEQ ID NO: 2387)
GUGgugauuc,
(SEQ ID NO: 2388)
GUGgugcgau,
(SEQ ID NO: 2389)
GUGgugcuua,
(SEQ ID NO: 2390)
GUGgugggaa,
(SEQ ID NO: 2391)
GUGgugggua,
(SEQ ID NO: 2392)
GUGguggguc,
(SEQ ID NO: 2393)
GUGguguccg,
(SEQ ID NO: 2394)
GUGguuagca,
(SEQ ID NO: 2395)
GUGguuaggu,
(SEQ ID NO: 2396)
GUGguuagug,
(SEQ ID NO: 2397)
GUGguuugca,
(SEQ ID NO: 2398)
GUGguuugua,
(SEQ ID NO: 2399)
GUUguaaggu,
(SEQ ID NO: 2400)
GUUguaagua,
(SEQ ID NO: 2401)
GUUguaaguc,
(SEQ ID NO: 2402)
GUUguaaguu,
(SEQ ID NO: 2403)
GUUguaccac,
(SEQ ID NO: 2404)
GUUguagcgu,
(SEQ ID NO: 2405)
GUUguaugug,
(SEQ ID NO: 2406)
GUUguauguu,
(SEQ ID NO: 2407)
GUUgucugug,
(SEQ ID NO: 2408)
GUUgugagcu,
(SEQ ID NO: 2409)
GUUgugagug,
(SEQ ID NO: 2410)
GUUgugaguu,
(SEQ ID NO: 2411)
GUUgugggua,
(SEQ ID NO: 2412)
GUUguggguu,
(SEQ ID NO: 2413)
UAAguaaaug,
(SEQ ID NO: 2414)
UAAguaacua,
(SEQ ID NO: 2415)
UAAguaagaa,
(SEQ ID NO: 2416)
UAAguaagag,
(SEQ ID NO: 2417)
UAAguaagau,
(SEQ ID NO: 2418)
UAAguaagca,
(SEQ ID NO: 2419)
UAAguaagcu,
(SEQ ID NO: 2420)
UAAguaagga,
(SEQ ID NO: 2421)
UAAguaaggu,
(SEQ ID NO: 2422)
UAAguaagua,
(SEQ ID NO: 2423)
UAAguaaguc,
(SEQ ID NO: 2424)
UAAguaagug,
(SEQ ID NO: 2425)
UAAguaaguu,
(SEQ ID NO: 2426)
UAAguaauaa,
(SEQ ID NO: 2427)
UAAguacuag,
(SEQ ID NO: 2428)
UAAguaguuu,
(SEQ ID NO: 2429)
UAAguauaaa,
(SEQ ID NO: 2430)
UAAguauaca,
(SEQ ID NO: 2431)
UAAguaugua,
(SEQ ID NO: 2432)
UAAguauuau,
(SEQ ID NO: 2433)
UAAguauuuu,
(SEQ ID NO: 2434)
UAAgucuuuu,
(SEQ ID NO: 2435)
UAAgugagac,
(SEQ ID NO: 2436)
UAAgugagga,
(SEQ ID NO: 2437)
UAAgugaggg,
(SEQ ID NO: 2438)
UAAgugagua,
(SEQ ID NO: 2439)
UAAgugaguc,
(SEQ ID NO: 2440)
UAAgugagug,
(SEQ ID NO: 2441)
UAAgugaguu,
(SEQ ID NO: 2442)
UAAgugaucc,
(SEQ ID NO: 2443)
UAAgugauuc,
(SEQ ID NO: 2444)
UAAgugcgug,
(SEQ ID NO: 2445)
UAAguuaagu,
(SEQ ID NO: 2446)
UAAguuccag,
(SEQ ID NO: 2447)
UAAguucuuu,
(SEQ ID NO: 2448)
UAAguuguaa,
(SEQ ID NO: 2449)
UAAguuguau,
(SEQ ID NO: 2450)
UAAguuuguu,
(SEQ ID NO: 2451)
UACguaacug,
(SEQ ID NO: 2452)
UACguaagaa,
(SEQ ID NO: 2453)
UACguaagau,
(SEQ ID NO: 2454)
UACguaagua,
(SEQ ID NO: 2455)
UACguaagug,
(SEQ ID NO: 2456)
UACguauccu,
(SEQ ID NO: 2457)
UACgucuggc,
(SEQ ID NO: 2458)
UACgugacca,
(SEQ ID NO: 2459)
UAGgcaagac,
(SEQ ID NO: 2460)
UAGgcaaguc,
(SEQ ID NO: 2461)
UAGgcagguc,
(SEQ ID NO: 2462)
UAGgcgugug,
(SEQ ID NO: 2463)
UAGguaaaaa,
(SEQ ID NO: 2464)
UAGguaaaac,
(SEQ ID NO: 2465)
UAGguaaaag,
(SEQ ID NO: 2466)
UAGguaaaau,
(SEQ ID NO: 2467)
UAGguaaaca,
(SEQ ID NO: 2468)
UAGguaaaga,
(SEQ ID NO: 2469)
UAGguaaaua,
(SEQ ID NO: 2470)
UAGguaaauc,
(SEQ ID NO: 2471)
UAGguaaaug,
(SEQ ID NO: 2472)
UAGguaaauu,
(SEQ ID NO: 2473)
UAGguaacac,
(SEQ ID NO: 2474)
UAGguaacag,
(SEQ ID NO: 2475)
UAGguaacau,
(SEQ ID NO: 2476)
UAGguaacca,
(SEQ ID NO: 2477)
UAGguaacgg,
(SEQ ID NO: 2478)
UAGguaacua,
(SEQ ID NO: 2479)
UAGguaacuc,
(SEQ ID NO: 2480)
UAGguaacug,
(SEQ ID NO: 2481)
UAGguaacuu,
(SEQ ID NO: 2482)
UAGguaagac,
(SEQ ID NO: 2483)
UAGguaagag,
(SEQ ID NO: 2484)
UAGguaagau,
(SEQ ID NO: 2485)
UAGguaagca,
(SEQ ID NO: 2486)
UAGguaagcc,
(SEQ ID NO: 2487)
UAGguaagcu,
(SEQ ID NO: 2488)
UAGguaagga,
(SEQ ID NO: 2489)
UAGguaaggc,
(SEQ ID NO: 2490)
UAGguaaggg,
(SEQ ID NO: 2491)
UAGguaagua,
(SEQ ID NO: 2492)
UAGguaaguc,
(SEQ ID NO: 2493)
UAGguaagug,
(SEQ ID NO: 2494)
UAGguaaguu,
(SEQ ID NO: 2495)
UAGguaauag,
(SEQ ID NO: 2496)
UAGguaauau,
(SEQ ID NO: 2497)
UAGguaaucu,
(SEQ ID NO: 2498)
UAGguaauga,
(SEQ ID NO: 2499)
UAGguaaugg,
(SEQ ID NO: 2500)
UAGguaaugu,
(SEQ ID NO: 2501)
UAGguaauua,
(SEQ ID NO: 2502)
UAGguaauuc,
(SEQ ID NO: 2503)
UAGguaauuu,
(SEQ ID NO: 2504)
UAGguacagc,
(SEQ ID NO: 2505)
UAGguacagu,
(SEQ ID NO: 2506)
UAGguacauu,
(SEQ ID NO: 2507)
UAGguaccag,
(SEQ ID NO: 2508)
UAGguaccua,
(SEQ ID NO: 2509)
UAGguaccuu,
(SEQ ID NO: 2510)
UAGguacgag,
(SEQ ID NO: 2511)
UAGguacgua,
(SEQ ID NO: 2512)
UAGguacguu,
(SEQ ID NO: 2513)
UAGguacuau,
(SEQ ID NO: 2514)
UAGguacuga,
(SEQ ID NO: 2515)
UAGguacugg,
(SEQ ID NO: 2516)
UAGguacuuc,
(SEQ ID NO: 2517)
UAGguacuuu,
(SEQ ID NO: 2518)
UAGguagcgg,
(SEQ ID NO: 2519)
UAGguaggaa,
(SEQ ID NO: 2520)
UAGguaggac,
(SEQ ID NO: 2521)
UAGguaggau,
(SEQ ID NO: 2522)
UAGguaggga,
(SEQ ID NO: 2523)
UAGguagggg,
(SEQ ID NO: 2524)
UAGguaggua,
(SEQ ID NO: 2525)
UAGguagguc,
(SEQ ID NO: 2526)
UAGguaggug,
(SEQ ID NO: 2527)
UAGguagguu,
(SEQ ID NO: 2528)
UAGguaguaa,
(SEQ ID NO: 2529)
UAGguagucu,
(SEQ ID NO: 2530)
UAGguagugg,
(SEQ ID NO: 2531)
UAGguagugu,
(SEQ ID NO: 2532)
UAGguaguuu,
(SEQ ID NO: 2533)
UAGguauaaa,
(SEQ ID NO: 2534)
UAGguauaac,
(SEQ ID NO: 2535)
UAGguauaag,
(SEQ ID NO: 2536)
UAGguauaau,
(SEQ ID NO: 2537)
UAGguauaca,
(SEQ ID NO: 2538)
UAGguauacu,
(SEQ ID NO: 2539)
UAGguauaua,
(SEQ ID NO: 2540)
UAGguauauc,
(SEQ ID NO: 2541)
UAGguauauu,
(SEQ ID NO: 2542)
UAGguaucag,
(SEQ ID NO: 2543)
UAGguaucua,
(SEQ ID NO: 2544)
UAGguaucuc,
(SEQ ID NO: 2545)
UAGguaugaa,
(SEQ ID NO: 2546)
UAGguaugag,
(SEQ ID NO: 2547)
UAGguaugca,
(SEQ ID NO: 2548)
UAGguaugga,
(SEQ ID NO: 2549)
UAGguauggc,
(SEQ ID NO: 2550)
UAGguauggu,
(SEQ ID NO: 2551)
UAGguaugua,
(SEQ ID NO: 2552)
UAGguauguc,
(SEQ ID NO: 2553)
UAGguaugug,
(SEQ ID NO: 2554)
UAGguauguu,
(SEQ ID NO: 2555)
UAGguauuaa,
(SEQ ID NO: 2556)
UAGguauuac,
(SEQ ID NO: 2557)
UAGguauuau,
(SEQ ID NO: 2558)
UAGguauuca,
(SEQ ID NO: 2559)
UAGguauucc,
(SEQ ID NO: 2560)
UAGguauucu,
(SEQ ID NO: 2561)
UAGguauuga,
(SEQ ID NO: 2562)
UAGguauuua,
(SEQ ID NO: 2563)
UAGguauuuc,
(SEQ ID NO: 2564)
UAGguauuuu,
(SEQ ID NO: 2565)
UAGgucacuc,
(SEQ ID NO: 2566)
UAGgucagcu,
(SEQ ID NO: 2567)
UAGgucaggu,
(SEQ ID NO: 2568)
UAGgucagua,
(SEQ ID NO: 2569)
UAGgucagug,
(SEQ ID NO: 2570)
UAGgucaguu,
(SEQ ID NO: 2571)
UAGgucaucu,
(SEQ ID NO: 2572)
UAGgucauug,
(SEQ ID NO: 2573)
UAGguccaau,
(SEQ ID NO: 2574)
UAGguccugu,
(SEQ ID NO: 2575)
UAGgucucaa,
(SEQ ID NO: 2576)
UAGgucucgc,
(SEQ ID NO: 2577)
UAGgucuggc,
(SEQ ID NO: 2578)
UAGgucuguc,
(SEQ ID NO: 2579)
UAGgucugug,
(SEQ ID NO: 2580)
UAGgugaagu,
(SEQ ID NO: 2581)
UAGgugaaua,
(SEQ ID NO: 2582)
UAGgugaaug,
(SEQ ID NO: 2583)
UAGgugaauu,
(SEQ ID NO: 2584)
UAGgugacau,
(SEQ ID NO: 2585)
UAGgugacca,
(SEQ ID NO: 2586)
UAGgugacua,
(SEQ ID NO: 2587)
UAGgugagaa,
(SEQ ID NO: 2588)
UAGgugagac,
(SEQ ID NO: 2589)
UAGgugagag,
(SEQ ID NO: 2590)
UAGgugagau,
(SEQ ID NO: 2591)
UAGgugagcc,
(SEQ ID NO: 2592)
UAGgugagcu,
(SEQ ID NO: 2593)
UAGgugagga,
(SEQ ID NO: 2594)
UAGgugaggc,
(SEQ ID NO: 2595)
UAGgugaggu,
(SEQ ID NO: 2596)
UAGgugagua,
(SEQ ID NO: 2597)
UAGgugaguc,
(SEQ ID NO: 2598)
UAGgugagug,
(SEQ ID NO: 2599)
UAGgugauca,
(SEQ ID NO: 2600)
UAGgugauuc,
(SEQ ID NO: 2601)
UAGgugauuu,
(SEQ ID NO: 2602)
UAGgugcaua,
(SEQ ID NO: 2603)
UAGgugcauc,
(SEQ ID NO: 2604)
UAGgugccgu,
(SEQ ID NO: 2605)
UAGgugccug,
(SEQ ID NO: 2606)
UAGgugcgca,
(SEQ ID NO: 2607)
UAGgugcgua,
(SEQ ID NO: 2608)
UAGgugcgug,
(SEQ ID NO: 2609)
UAGgugcuga,
(SEQ ID NO: 2610)
UAGguggaua,
(SEQ ID NO: 2611)
UAGgugggaa,
(SEQ ID NO: 2612)
UAGgugggac,
(SEQ ID NO: 2613)
UAGgugggag,
(SEQ ID NO: 2614)
UAGgugggau,
(SEQ ID NO: 2615)
UAGgugggcc,
(SEQ ID NO: 2616)
UAGgugggcu,
(SEQ ID NO: 2617)
UAGguggguu,
(SEQ ID NO: 2618)
UAGguggugu,
(SEQ ID NO: 2619)
UAGguguaaa,
(SEQ ID NO: 2620)
UAGgugugaa,
(SEQ ID NO: 2621)
UAGgugugag,
(SEQ ID NO: 2622)
UAGgugugca,
(SEQ ID NO: 2623)
UAGgugugcc,
(SEQ ID NO: 2624)
UAGgugugcg,
(SEQ ID NO: 2625)
UAGguguggu,
(SEQ ID NO: 2626)
UAGgugugua,
(SEQ ID NO: 2627)
UAGgugugug,
(SEQ ID NO: 2628)
UAGguguugg,
(SEQ ID NO: 2629)
UAGguuaagc,
(SEQ ID NO: 2630)
UAGguuagac,
(SEQ ID NO: 2631)
UAGguuagcc,
(SEQ ID NO: 2632)
UAGguuaggc,
(SEQ ID NO: 2633)
UAGguuagua,
(SEQ ID NO: 2634)
UAGguuaguc,
(SEQ ID NO: 2635)
UAGguuagug,
(SEQ ID NO: 2636)
UAGguucccc,
(SEQ ID NO: 2637)
UAGguucuac,
(SEQ ID NO: 2638)
UAGguuggua,
(SEQ ID NO: 2639)
UAGguugguu,
(SEQ ID NO: 2640)
UAGguugucc,
(SEQ ID NO: 2641)
UAGguuuauu,
(SEQ ID NO: 2642)
UAGguuugcc,
(SEQ ID NO: 2643)
UAGguuugua,
(SEQ ID NO: 2644)
UAGguuuguc,
(SEQ ID NO: 2645)
UAGguuugug,
(SEQ ID NO: 2646)
UAGguuuguu,
(SEQ ID NO: 2647)
UAGguuuuuc,
(SEQ ID NO: 2648)
UAGguuuuug,
(SEQ ID NO: 2649)
UAUguaagaa,
(SEQ ID NO: 2650)
UAUguaagau,
(SEQ ID NO: 2651)
UAUguaagca,
(SEQ ID NO: 2652)
UAUguaagcc,
(SEQ ID NO: 2653)
UAUguaagua,
(SEQ ID NO: 2654)
UAUguaaguc,
(SEQ ID NO: 2655)
UAUguaagug,
(SEQ ID NO: 2656)
UAUguaaguu,
(SEQ ID NO: 2657)
UAUguacgug,
(SEQ ID NO: 2658)
UAUguacguu,
(SEQ ID NO: 2659)
UAUguagguc,
(SEQ ID NO: 2660)
UAUguagguu,
(SEQ ID NO: 2661)
UAUguauccu,
(SEQ ID NO: 2662)
UAUguaucuc,
(SEQ ID NO: 2663)
UAUguaugua,
(SEQ ID NO: 2664)
UAUguauguc,
(SEQ ID NO: 2665)
UAUguaugug,
(SEQ ID NO: 2666)
UAUguauuau,
(SEQ ID NO: 2667)
UAUgucagaa,
(SEQ ID NO: 2668)
UAUgucugua,
(SEQ ID NO: 2669)
UAUgugaaua,
(SEQ ID NO: 2670)
UAUgugacag,
(SEQ ID NO: 2671)
UAUgugagua,
(SEQ ID NO: 2672)
UAUgugagug,
(SEQ ID NO: 2673)
UAUgugaguu,
(SEQ ID NO: 2674)
UAUgugggca,
(SEQ ID NO: 2675)
UAUgugugua,
(SEQ ID NO: 2676)
UAUguguuua,
(SEQ ID NO: 2677)
UAUguuuugu,
(SEQ ID NO: 2678)
UCAgcgacau,
(SEQ ID NO: 2679)
UCAguaaaau,
(SEQ ID NO: 2680)
UCAguaaaua,
(SEQ ID NO: 2681)
UCAguaacug,
(SEQ ID NO: 2682)
UCAguaagaa,
(SEQ ID NO: 2683)
UCAguaagag,
(SEQ ID NO: 2684)
UCAguaagau,
(SEQ ID NO: 2685)
UCAguaagca,
(SEQ ID NO: 2686)
UCAguaagcc,
(SEQ ID NO: 2687)
UCAguaagcu,
(SEQ ID NO: 2688)
UCAguaaggg,
(SEQ ID NO: 2689)
UCAguaagua,
(SEQ ID NO: 2690)
UCAguaaguc,
(SEQ ID NO: 2691)
UCAguaagug,
(SEQ ID NO: 2692)
UCAguaaguu,
(SEQ ID NO: 2693)
UCAguaucuu,
(SEQ ID NO: 2694)
UCAguaugga,
(SEQ ID NO: 2695)
UCAguauggu,
(SEQ ID NO: 2696)
UCAgucccca,
(SEQ ID NO: 2697)
UCAgugagca,
(SEQ ID NO: 2698)
UCAgugagcu,
(SEQ ID NO: 2699)
UCAgugagua,
(SEQ ID NO: 2700)
UCAgugagug,
(SEQ ID NO: 2701)
UCAgugaguu,
(SEQ ID NO: 2702)
UCAgugauug,
(SEQ ID NO: 2703)
UCAgugggug,
(SEQ ID NO: 2704)
UCAguugagc,
(SEQ ID NO: 2705)
UCAguugauu,
(SEQ ID NO: 2706)
UCAguuuagu,
(SEQ ID NO: 2707)
UCCguaagca,
(SEQ ID NO: 2708)
UCCguaagcu,
(SEQ ID NO: 2709)
UCCguaaguc,
(SEQ ID NO: 2710)
UCCguaagug,
(SEQ ID NO: 2711)
UCCguaauag,
(SEQ ID NO: 2712)
UCCguacuua,
(SEQ ID NO: 2713)
UCCguaugua,
(SEQ ID NO: 2714)
UCCguauguu,
(SEQ ID NO: 2715)
UCCgugagau,
(SEQ ID NO: 2716)
UCCgugaguc,
(SEQ ID NO: 2717)
UCGguaaauu,
(SEQ ID NO: 2718)
UCGguaagag,
(SEQ ID NO: 2719)
UCGguaagcu,
(SEQ ID NO: 2720)
UCGguacauc,
(SEQ ID NO: 2721)
UCGguacucc,
(SEQ ID NO: 2722)
UCGguagacc,
(SEQ ID NO: 2723)
UCGguagguu,
(SEQ ID NO: 2724)
UCGguaguaa,
(SEQ ID NO: 2725)
UCGguaugug,
(SEQ ID NO: 2726)
UCGguauguu,
(SEQ ID NO: 2727)
UCGguauuga,
(SEQ ID NO: 2728)
UCGgucagua,
(SEQ ID NO: 2729)
UCGgucuuag,
(SEQ ID NO: 2730)
UCGgugaagu,
(SEQ ID NO: 2731)
UCGgugagaa,
(SEQ ID NO: 2732)
UCGgugagca,
(SEQ ID NO: 2733)
UCGgugaggc,
(SEQ ID NO: 2734)
UCGgugagua,
(SEQ ID NO: 2735)
UCGgugcgcu,
(SEQ ID NO: 2736)
UCGgugcuuu,
(SEQ ID NO: 2737)
UCGgugguuu,
(SEQ ID NO: 2738)
UCGguuagcu,
(SEQ ID NO: 2739)
UCUguaaaag,
(SEQ ID NO: 2740)
UCUguaagaa,
(SEQ ID NO: 2741)
UCUguaagau,
(SEQ ID NO: 2742)
UCUguaagca,
(SEQ ID NO: 2743)
UCUguaagcu,
(SEQ ID NO: 2744)
UCUguaagua,
(SEQ ID NO: 2745)
UCUguaaguc,
(SEQ ID NO: 2746)
UCUguaagug,
(SEQ ID NO: 2747)
UCUguaaguu,
(SEQ ID NO: 2748)
UCUguaauaa,
(SEQ ID NO: 2749)
UCUguaauga,
(SEQ ID NO: 2750)
UCUguaaugu,
(SEQ ID NO: 2751)
UCUguaggua,
(SEQ ID NO: 2752)
UCUguagguu,
(SEQ ID NO: 2753)
UCUguauaua,
(SEQ ID NO: 2754)
UCUguaugac,
(SEQ ID NO: 2755)
UCUguaugua,
(SEQ ID NO: 2756)
UCUguccucg,
(SEQ ID NO: 2757)
UCUgugagag,
(SEQ ID NO: 2758)
UCUgugagcu,
(SEQ ID NO: 2759)
UCUgugagga,
(SEQ ID NO: 2760)
UCUgugagua,
(SEQ ID NO: 2761)
UCUgugaguc,
(SEQ ID NO: 2762)
UCUgugagug,
(SEQ ID NO: 2763)
UCUgugaguu,
(SEQ ID NO: 2764)
UCUgugcgua,
(SEQ ID NO: 2765)
UCUgugugag,
(SEQ ID NO: 2766)
UGAguaacuu,
(SEQ ID NO: 2767)
UGAguaagau,
(SEQ ID NO: 2768)
UGAguaagca,
(SEQ ID NO: 2769)
UGAguaagcu,
(SEQ ID NO: 2770)
UGAguaaggc,
(SEQ ID NO: 2771)
UGAguaaggu,
(SEQ ID NO: 2772)
UGAguaagua,
(SEQ ID NO: 2773)
UGAguaaguc,
(SEQ ID NO: 2774)
UGAguaagug,
(SEQ ID NO: 2775)
UGAguaaguu,
(SEQ ID NO: 2776)
UGAguaaucc,
(SEQ ID NO: 2777)
UGAguaauua,
(SEQ ID NO: 2778)
UGAguacagu,
(SEQ ID NO: 2779)
UGAguacgua,
(SEQ ID NO: 2780)
UGAguacguu,
(SEQ ID NO: 2781)
UGAguacugu,
(SEQ ID NO: 2782)
UGAguagcug,
(SEQ ID NO: 2783)
UGAguaggua,
(SEQ ID NO: 2784)
UGAguauaaa,
(SEQ ID NO: 2785)
UGAguaugcu,
(SEQ ID NO: 2786)
UGAguaugga,
(SEQ ID NO: 2787)
UGAguaugua,
(SEQ ID NO: 2788)
UGAguauguc,
(SEQ ID NO: 2789)
UGAguauguu,
(SEQ ID NO: 2790)
UGAgucagag,
(SEQ ID NO: 2791)
UGAgucuacg,
(SEQ ID NO: 2792)
UGAgugaaua,
(SEQ ID NO: 2793)
UGAgugaauu,
(SEQ ID NO: 2794)
UGAgugagaa,
(SEQ ID NO: 2795)
UGAgugagau,
(SEQ ID NO: 2796)
UGAgugagca,
(SEQ ID NO: 2797)
UGAgugagcc,
(SEQ ID NO: 2798)
UGAgugagga,
(SEQ ID NO: 2799)
UGAgugagua,
(SEQ ID NO: 2800)
UGAgugagug,
(SEQ ID NO: 2801)
UGAgugaguu,
(SEQ ID NO: 2802)
UGAgugggaa,
(SEQ ID NO: 2803)
UGAguuaaga,
(SEQ ID NO: 2804)
UGAguuaaug,
(SEQ ID NO: 2805)
UGAguuacgg,
(SEQ ID NO: 2806)
UGAguuaggu,
(SEQ ID NO: 2807)
UGAguucuau,
(SEQ ID NO: 2808)
UGAguugguu,
(SEQ ID NO: 2809)
UGAguuguag,
(SEQ ID NO: 2810)
UGAguuuauc,
(SEQ ID NO: 2811)
UGCguaaguc,
(SEQ ID NO: 2812)
UGCguaagug,
(SEQ ID NO: 2813)
UGCguacggc,
(SEQ ID NO: 2814)
UGCguacggg,
(SEQ ID NO: 2815)
UGCguaugua,
(SEQ ID NO: 2816)
UGGgcaaguc,
(SEQ ID NO: 2817)
UGGgcaagug,
(SEQ ID NO: 2818)
UGGgcacauc,
(SEQ ID NO: 2819)
UGGgccacgu,
(SEQ ID NO: 2820)
UGGgccccgg,
(SEQ ID NO: 2821)
UGGguaaaau,
(SEQ ID NO: 2822)
UGGguaaagc,
(SEQ ID NO: 2823)
UGGguaaagg,
(SEQ ID NO: 2824)
UGGguaaagu,
(SEQ ID NO: 2825)
UGGguaaaua,
(SEQ ID NO: 2826)
UGGguaaaug,
(SEQ ID NO: 2827)
UGGguaaauu,
(SEQ ID NO: 2828)
UGGguaacag,
(SEQ ID NO: 2829)
UGGguaacau,
(SEQ ID NO: 2830)
UGGguaacua,
(SEQ ID NO: 2831)
UGGguaacuu,
(SEQ ID NO: 2832)
UGGguaagaa,
(SEQ ID NO: 2833)
UGGguaagac,
(SEQ ID NO: 2834)
UGGguaagag,
(SEQ ID NO: 2835)
UGGguaagau,
(SEQ ID NO: 2836)
UGGguaagca,
(SEQ ID NO: 2837)
UGGguaagcc,
(SEQ ID NO: 2838)
UGGguaagcu,
(SEQ ID NO: 2839)
UGGguaaggg,
(SEQ ID NO: 2840)
UGGguaaggu,
(SEQ ID NO: 2841)
UGGguaagua,
(SEQ ID NO: 2842)
UGGguaaguc,
(SEQ ID NO: 2843)
UGGguaagug,
(SEQ ID NO: 2844)
UGGguaaguu,
(SEQ ID NO: 2845)
UGGguaaugu,
(SEQ ID NO: 2846)
UGGguaauua,
(SEQ ID NO: 2847)
UGGguaauuu,
(SEQ ID NO: 2848)
UGGguacaaa,
(SEQ ID NO: 2849)
UGGguacagu,
(SEQ ID NO: 2850)
UGGguacuac,
(SEQ ID NO: 2851)
UGGguaggga,
(SEQ ID NO: 2852)
UGGguagguc,
(SEQ ID NO: 2853)
UGGguaggug,
(SEQ ID NO: 2854)
UGGguagguu,
(SEQ ID NO: 2855)
UGGguaguua,
(SEQ ID NO: 2856)
UGGguauagu,
(SEQ ID NO: 2857)
UGGguaugaa,
(SEQ ID NO: 2858)
UGGguaugac,
(SEQ ID NO: 2859)
UGGguaugag,
(SEQ ID NO: 2860)
UGGguaugua,
(SEQ ID NO: 2861)
UGGguauguc,
(SEQ ID NO: 2862)
UGGguaugug,
(SEQ ID NO: 2863)
UGGguauguu,
(SEQ ID NO: 2864)
UGGguauuug,
(SEQ ID NO: 2865)
UGGgucuuug,
(SEQ ID NO: 2866)
UGGgugaccu,
(SEQ ID NO: 2867)
UGGgugacua,
(SEQ ID NO: 2868)
UGGgugagac,
(SEQ ID NO: 2869)
UGGgugagag,
(SEQ ID NO: 2870)
UGGgugagca,
(SEQ ID NO: 2871)
UGGgugagcc,
(SEQ ID NO: 2872)
UGGgugagga,
(SEQ ID NO: 2873)
UGGgugaggc,
(SEQ ID NO: 2874)
UGGgugaggg,
(SEQ ID NO: 2875)
UGGgugagua,
(SEQ ID NO: 2876)
UGGgugaguc,
(SEQ ID NO: 2877)
UGGgugagug,
(SEQ ID NO: 2878)
UGGgugaguu,
(SEQ ID NO: 2879)
UGGgugcgug,
(SEQ ID NO: 2880)
UGGguggagg,
(SEQ ID NO: 2881)
UGGguggcuu,
(SEQ ID NO: 2882)
UGGguggggg,
(SEQ ID NO: 2883)
UGGgugggua,
(SEQ ID NO: 2884)
UGGguggguc,
(SEQ ID NO: 2885)
UGGgugggug,
(SEQ ID NO: 2886)
UGGguggguu,
(SEQ ID NO: 2887)
UGGgugugga,
(SEQ ID NO: 2888)
UGGguguguc,
(SEQ ID NO: 2889)
UGGgugugug,
(SEQ ID NO: 2890)
UGGguguguu,
(SEQ ID NO: 2891)
UGGguguuua,
(SEQ ID NO: 2892)
UGGguuaaug,
(SEQ ID NO: 2893)
UGGguuaguc,
(SEQ ID NO: 2894)
UGGguuagug,
(SEQ ID NO: 2895)
UGGguuaguu,
(SEQ ID NO: 2896)
UGGguucaag,
(SEQ ID NO: 2897)
UGGguucgua,
(SEQ ID NO: 2898)
UGGguuggug,
(SEQ ID NO: 2899)
UGGguuuaag,
(SEQ ID NO: 2900)
UGGguuugua,
(SEQ ID NO: 2901)
UGUgcaagua,
(SEQ ID NO: 2902)
UGUguaaaua,
(SEQ ID NO: 2903)
UGUguaagaa,
(SEQ ID NO: 2904)
UGUguaagac,
(SEQ ID NO: 2905)
UGUguaagag,
(SEQ ID NO: 2906)
UGUguaaggu,
(SEQ ID NO: 2907)
UGUguaagua,
(SEQ ID NO: 2908)
UGUguaaguc,
(SEQ ID NO: 2909)
UGUguaaguu,
(SEQ ID NO: 2910)
UGUguacuuc,
(SEQ ID NO: 2911)
UGUguaggcg,
(SEQ ID NO: 2912)
UGUguaggua,
(SEQ ID NO: 2913)
UGUguaguua,
(SEQ ID NO: 2914)
UGUguaugug,
(SEQ ID NO: 2915)
UGUgucagua,
(SEQ ID NO: 2916)
UGUgucugua,
(SEQ ID NO: 2917)
UGUgucuguc,
(SEQ ID NO: 2918)
UGUgugaccc,
(SEQ ID NO: 2919)
UGUgugagau,
(SEQ ID NO: 2920)
UGUgugagca,
(SEQ ID NO: 2921)
UGUgugagcc,
(SEQ ID NO: 2922)
UGUgugagua,
(SEQ ID NO: 2923)
UGUgugaguc,
(SEQ ID NO: 2924)
UGUgugagug,
(SEQ ID NO: 2925)
UGUgugcgug,
(SEQ ID NO: 2926)
UGUgugggug,
(SEQ ID NO: 2927)
UGUguggguu,
(SEQ ID NO: 2928)
UGUgugugag,
(SEQ ID NO: 2929)
UGUguguucu,
(SEQ ID NO: 2930)
UGUguuuaga,
(SEQ ID NO: 2931)
UUAguaaaua,
(SEQ ID NO: 2932)
UUAguaagaa,
(SEQ ID NO: 2933)
UUAguaagua,
(SEQ ID NO: 2934)
UUAguaagug,
(SEQ ID NO: 2935)
UUAguaaguu,
(SEQ ID NO: 2936)
UUAguaggug,
(SEQ ID NO: 2937)
UUAgugagca,
(SEQ ID NO: 2938)
UUAgugaguu,
(SEQ ID NO: 2939)
UUAguuaagu,
(SEQ ID NO: 2940)
UUCguaaguc,
(SEQ ID NO: 2941)
UUCguaaguu,
(SEQ ID NO: 2942)
UUCguaauua,
(SEQ ID NO: 2943)
UUCgugagua,
(SEQ ID NO: 2944)
UUCgugaguu,
(SEQ ID NO: 2945)
UUGgcaagug,
(SEQ ID NO: 2946)
UUGgccgagu,
(SEQ ID NO: 2947)
UUGguaaaaa,
(SEQ ID NO: 2948)
UUGguaaaau,
(SEQ ID NO: 2949)
UUGguaaaga,
(SEQ ID NO: 2950)
UUGguaaagg,
(SEQ ID NO: 2951)
UUGguaaagu,
(SEQ ID NO: 2952)
UUGguaaauc,
(SEQ ID NO: 2953)
UUGguaaaug,
(SEQ ID NO: 2954)
UUGguaaauu,
(SEQ ID NO: 2955)
UUGguaacug,
(SEQ ID NO: 2956)
UUGguaacuu,
(SEQ ID NO: 2957)
UUGguaagaa,
(SEQ ID NO: 2958)
UUGguaagag,
(SEQ ID NO: 2959)
UUGguaagcu,
(SEQ ID NO: 2960)
UUGguaagga,
(SEQ ID NO: 2961)
UUGguaaggg,
(SEQ ID NO: 2962)
UUGguaagua,
(SEQ ID NO: 2963)
UUGguaagug,
(SEQ ID NO: 2964)
UUGguaaguu,
(SEQ ID NO: 2965)
UUGguaauac,
(SEQ ID NO: 2966)
UUGguaauca,
(SEQ ID NO: 2967)
UUGguaaugc,
(SEQ ID NO: 2968)
UUGguaaugu,
(SEQ ID NO: 2969)
UUGguaauug,
(SEQ ID NO: 2970)
UUGguaauuu,
(SEQ ID NO: 2971)
UUGguacaua,
(SEQ ID NO: 2972)
UUGguacgug,
(SEQ ID NO: 2973)
UUGguagagg,
(SEQ ID NO: 2974)
UUGguaggac,
(SEQ ID NO: 2975)
UUGguaggcg,
(SEQ ID NO: 2976)
UUGguaggcu,
(SEQ ID NO: 2977)
UUGguaggga,
(SEQ ID NO: 2978)
UUGguaggua,
(SEQ ID NO: 2979)
UUGguagguc,
(SEQ ID NO: 2980)
UUGguaggug,
(SEQ ID NO: 2981)
UUGguauaaa,
(SEQ ID NO: 2982)
UUGguauaca,
(SEQ ID NO: 2983)
UUGguauauu,
(SEQ ID NO: 2984)
UUGguaucua,
(SEQ ID NO: 2985)
UUGguaucuc,
(SEQ ID NO: 2986)
UUGguaugca,
(SEQ ID NO: 2987)
UUGguaugua,
(SEQ ID NO: 2988)
UUGguaugug,
(SEQ ID NO: 2989)
UUGguauguu,
(SEQ ID NO: 2990)
UUGguauugu,
(SEQ ID NO: 2991)
UUGguauuua,
(SEQ ID NO: 2992)
UUGguauuuu,
(SEQ ID NO: 2993)
UUGgucagaa,
(SEQ ID NO: 2994)
UUGgucagua,
(SEQ ID NO: 2995)
UUGgucucug,
(SEQ ID NO: 2996)
UUGgucugca,
(SEQ ID NO: 2997)
UUGgugaaaa,
(SEQ ID NO: 2998)
UUGgugacug,
(SEQ ID NO: 2999)
UUGgugagac,
(SEQ ID NO: 3000)
UUGgugagau,
(SEQ ID NO: 3001)
UUGgugagca,
(SEQ ID NO: 3002)
UUGgugagga,
(SEQ ID NO: 3003)
UUGgugaggg,
(SEQ ID NO: 3004)
UUGgugagua,
(SEQ ID NO: 3005)
UUGgugaguc,
(SEQ ID NO: 3006)
UUGgugagug,
(SEQ ID NO: 3007)
UUGgugaguu,
(SEQ ID NO: 3008)
UUGgugaugg,
(SEQ ID NO: 3009)
UUGgugauua,
(SEQ ID NO: 3010)
UUGgugauug,
(SEQ ID NO: 3011)
UUGgugcaca,
(SEQ ID NO: 3012)
UUGgugggaa,
(SEQ ID NO: 3013)
UUGguggggc,
(SEQ ID NO: 3014)
UUGgugggua,
(SEQ ID NO: 3015)
UUGguggguc,
(SEQ ID NO: 3016)
UUGgugggug,
(SEQ ID NO: 3017)
UUGguggguu,
(SEQ ID NO: 3018)
UUGguguggu,
(SEQ ID NO: 3019)
UUGguguguc,
(SEQ ID NO: 3020)
UUGgugugug,
(SEQ ID NO: 3021)
UUGguguguu,
(SEQ ID NO: 3022)
UUGguuaagu,
(SEQ ID NO: 3023)
UUGguuagca,
(SEQ ID NO: 3024)
UUGguuagug,
(SEQ ID NO: 3025)
UUGguuaguu,
(SEQ ID NO: 3026)
UUGguuggga,
(SEQ ID NO: 3027)
UUGguugguu,
(SEQ ID NO: 3028)
UUGguuugua,
(SEQ ID NO: 3029)
UUGguuuguc,
(SEQ ID NO: 3030)
UUUgcaagug,
(SEQ ID NO: 3031)
UUUguaaaua,
(SEQ ID NO: 3032)
UUUguaaaug,
(SEQ ID NO: 3033)
UUUguaagaa,
(SEQ ID NO: 3034)
UUUguaagac,
(SEQ ID NO: 3035)
UUUguaagag,
(SEQ ID NO: 3036)
UUUguaagca,
(SEQ ID NO: 3037)
UUUguaaggu,
(SEQ ID NO: 3038)
UUUguaagua,
(SEQ ID NO: 3039)
UUUguaaguc,
(SEQ ID NO: 3040)
UUUguaagug,
(SEQ ID NO: 3041)
UUUguaaguu,
(SEQ ID NO: 3042)
UUUguaauuu,
(SEQ ID NO: 3043)
UUUguacagg,
(SEQ ID NO: 3044)
UUUguacgug,
(SEQ ID NO: 3045)
UUUguacuag,
(SEQ ID NO: 3046)
UUUguacugu,
(SEQ ID NO: 3047)
UUUguagguu,
(SEQ ID NO: 3048)
UUUguauccu,
(SEQ ID NO: 3049)
UUUguauguu,
(SEQ ID NO: 3050)
UUUgugagca,
(SEQ ID NO: 3051)
UUUgugagug,
(SEQ ID NO: 3052)
UUUgugcguc,
(SEQ ID NO: 3053)
UUUguguguc,
and
(SEQ ID NO: 3054)
uGGguaccug.

Additional exemplary gene sequences and splice site sequences (e.g., 5′ splice site sequences) include

(SEQ ID NO: 96)
AAGgcaagau,
(SEQ ID NO: 937)
AUGguaugug,
(SEQ ID NO: 2281)
GGGgugaggc,
(SEQ ID NO: 1222)
CAGguaggug,
(SEQ ID NO: 293)
AAGgucagua,
(SEQ ID NO: 3055)
AAGguuagag
(SEQ ID NO: 3056)
AUGgcacuua,
(SEQ ID NO: 2423)
UAAguaaguc,
(SEQ ID NO: 3057)
UGGgugagcu,
(SEQ ID NO: 3058)
CGAgcugggc,
(SEQ ID NO: 3059)
AAAgcacccc,
(SEQ ID NO: 3060)
UAGguggggg,
(SEQ ID NO: 3061)
AGAguaacgu,
(SEQ ID NO: 3062)
UCGgugaugu,
(SEQ ID NO: 516)
AAUgucaguu,
(SEQ ID NO: 3063)
AGGgucugag,
(SEQ ID NO: 3064)
GAGgugacug,
(SEQ ID NO: 3065)
AUGguagguu,
(SEQ ID NO: 2000)
GAGgucuguc,
(SEQ ID NO: 1260)
CAGguaugug,
(SEQ ID NO: 3066)
CAAguacugc,
(SEQ ID NO: 3067)
CACgugcgua,
(SEQ ID NO: 3068)
CCGgugagcu,
(SEQ ID NO: 3069)
CAGguacuuc,
(SEQ ID NO: 1115)
CAGgcgagag,
(SEQ ID NO: 3070)
GAAgcaagua,
(SEQ ID NO: 789)
AGGgugagca,
(SEQ ID NO: 3071)
CAGgcaaguc,
(SEQ ID NO: 344)
AAGgugaggc,
(SEQ ID NO: 1147)
CAGguaagua,
(SEQ ID NO: 3072)
CCAguugggu,
(SEQ ID NO: 3073)
AAGguguggg,
(SEQ ID NO: 1484)
CAGguuggag,
(SEQ ID NO: 3074)
CCGguaugaa,
(SEQ ID NO: 2845)
UGGguaaugu,
(SEQ ID NO: 1344)
CAGgugaggu,
(SEQ ID NO: 3075)
AGAguaauag,
(SEQ ID NO: 1249)
CAGguaugag,
(SEQ ID NO: 901)
AUGguaaguu,
(SEQ ID NO: 3015)
UUGguggguc,
(SEQ ID NO: 3036)
UUUguaagca,
(SEQ ID NO: 3076)
CUCguaugcc,
(SEQ ID NO: 2483)
UAGguaagag,
(SEQ ID NO: 3077)
UAGgcaaguu,
(SEQ ID NO: 3078)
GGAguuaagu,
(SEQ ID NO: 1959)
GAGguaugcc,
(SEQ ID NO: 402)
AAGguguggu,
(SEQ ID NO: 1415)
CAGgugggug,
(SEQ ID NO: 2933)
UUAguaagua,
(SEQ ID NO: 3079)
AAGguuggcu,
(SEQ ID NO: 3080)
UGAguaugug,
(SEQ ID NO: 3081)
CCAgccuucc,
(SEQ ID NO: 3082)
CCUguacgug,
(SEQ ID NO: 1601)
CCUguaggua,
(SEQ ID NO: 3083)
CAGguacgcu,
(SEQ ID NO: 3084)
GAGguucuuc,
(SEQ ID NO: 3085)
AAGguugccu,
(SEQ ID NO: 3086)
CGUguucacu,
(SEQ ID NO: 3087)
CGGgugggga,
(SEQ ID NO: 2614)
UAGgugggau,
(SEQ ID NO: 3088)
CGGguaagga,
(SEQ ID NO: 195)
AAGguacuau,
(SEQ ID NO: 2248)
GGGguaagcu,
(SEQ ID NO: 3089)
ACGguagagc,
(SEQ ID NO: 1318)
CAGgugaaga,
(SEQ ID NO: 3090)
GCGguaagag,
(SEQ ID NO: 3091)
CAGguguugu,
(SEQ ID NO: 3092)
GAAguuugug,
(SEQ ID NO: 955)
AUGgugagca,
(SEQ ID NO: 3093)
CGGguucgug,
(SEQ ID NO: 3094)
AUUguccggc,
(SEQ ID NO: 3095)
GAUgugugug,
(SEQ ID NO: 3096)
AUGgucuguu,
(SEQ ID NO: 219)
AAGguaggau,
(SEQ ID NO: 1575)
CCGguaagau,
(SEQ ID NO: 126)
AAGguaaaga,
(SEQ ID NO: 2285)
GGGgugaguu,
(SEQ ID NO: 808)
AGGguuggug,
(SEQ ID NO: 2228)
GGAgugagug,
(SEQ ID NO: 3097)
AGUguaagga,
(SEQ ID NO: 2480)
UAGguaacug,
(SEQ ID NO: 3098)
AAGgugaaga,
(SEQ ID NO: 2843)
UGGguaagug,
(SEQ ID NO: 1140)
CAGguaagag,
(SEQ ID NO: 3099)
UAGgugagcg,
(SEQ ID NO: 1865)
GAGguaaaaa,
(SEQ ID NO: 3100)
GCCguaaguu,
(SEQ ID NO: 473)
AAGguuuugu,
(SEQ ID NO: 1341)
CAGgugagga,
(SEQ ID NO: 3101)
ACAgcccaug,
(SEQ ID NO: 3102)
GCGgugagcc,
(SEQ ID NO: 1251)
CAGguaugca,
(SEQ ID NO: 3103)
AUGguaccua,
(SEQ ID NO: 1050)
CAAguaugua,
(SEQ ID NO: 3104)
AUGguggugc,
(SEQ ID NO: 3105)
UAAguggcag,
(SEQ ID NO: 3106)
UAGguauagu,
(SEQ ID NO: 3107)
CUGguauuua,
(SEQ ID NO: 3108)
AGGguaaacg,
(SEQ ID NO: 850)
AUAguaagug,
(SEQ ID NO: 3109)
UUGguacuga,
(SEQ ID NO: 2303)
GGUguaagcc,
(SEQ ID NO: 3110)
GAGguggaua,
(SEQ ID NO: 3111)
GAUguaagaa,
(SEQ ID NO: 3112)
ACGgucaguu,
(SEQ ID NO: 3113)
UAAguaaaca,
(SEQ ID NO: 251)
AAGguaucug,
(SEQ ID NO: 3114)
AGGguauuug,
(SEQ ID NO: 328)
AAGgugaaug,
(SEQ ID NO: 1749)
CUGgugaauu,
(SEQ ID NO: 1514)
CAGguuuuuu,
(SEQ ID NO: 1534)
CAUguaugug,
(SEQ ID NO: 2973)
UUGguagagg,
(SEQ ID NO: 258)
AAGguaugcc,
(SEQ ID NO: 3115)
CAGgugccac,
(SEQ ID NO: 2727)
UCGguauuga,
(SEQ ID NO: 468)
AAGguuugug,
(SEQ ID NO: 3116)
AAUguacagg,
(SEQ ID NO: 1545)
CAUguggguu,
(SEQ ID NO: 1542)
CAUgugaguu,
(SEQ ID NO: 2968)
UUGguaaugu
(SEQ ID NO: 3117)
AGUguaggug,
(SEQ ID NO: 3118)
GAGguaacuc,
(SEQ ID NO: 3119)
GAGguggcgc,
(SEQ ID NO: 3120)
CUGguaauug,
(SEQ ID NO: 3121)
GAGguuugcu,
(SEQ ID NO: 3122)
UGUguacgug,
(SEQ ID NO: 2468)
UAGguaaaga,
(SEQ ID NO: 3123)
CUAguaggca,
(SEQ ID NO: 2761)
UCUgugaguc,
(SEQ ID NO: 3124)
UCUguaaggc,
(SEQ ID NO: 1509)
CAGguuugug,
(SEQ ID NO: 1935)
GAGguagggc,
(SEQ ID NO: 3125)
AAGguaacca,
(SEQ ID NO: 646)
ACUgugaguu,
(SEQ ID NO: 2495)
UAGguaauag,
(SEQ ID NO: 17)
AAAguaagcu,
(SEQ ID NO: 963)
AUGgugagug,
(SEQ ID NO: 2645)
UAGguuugug,
(SEQ ID NO: 3126)
AACguaggac,
(SEQ ID NO: 3127)
GUAgcaggua,
(SEQ ID NO: 3128)
GAGgucagac,
(SEQ ID NO: 3129)
AGGguaugaa,
(SEQ ID NO: 2089)
GAGguuagug,
(SEQ ID NO: 3130)
CAGgcacgug,
(SEQ ID NO: 3131)
GGGgcaagac,
(SEQ ID NO: 1441)
CAGguguguc,
(SEQ ID NO: 1265)
CAGguauuga,
(SEQ ID NO: 1259)
CAGguauguc,
(SEQ ID NO: 3132)
AAGgcaaggu,
(SEQ ID NO: 3133)
UUGgugaga,
(SEQ ID NO: 122)
AAGguaaaau,
(SEQ ID NO: 2251)
GGGguaagua,
(SEQ ID NO: 252)
AAGguaucuu,
(SEQ ID NO: 3134)
GACgugaguc,
(SEQ ID NO: 3135)
UAUguaugcu,
(SEQ ID NO: 199)
AAGguacugu,
(SEQ ID NO: 3136)
CAGgugaacu,
(SEQ ID NO: 3137)
CACguaaaug,
(SEQ ID NO: 3138)
AAGgugugau,
(SEQ ID NO: 3139)
GAAguauuug,
(SEQ ID NO: 3140)
AAGgucugug,
(SEQ ID NO: 3141)
AAGguggagg,
(SEQ ID NO: 244)
AAGguauaug,
(SEQ ID NO: 1477)
CAGguucuua,
(SEQ ID NO: 730)
AGGguaacca,
(SEQ ID NO: 1423)
CAGgugucac,
(SEQ ID NO: 3142)
AAAguucugu,
(SEQ ID NO: 3007)
UUGgugaguu,
(SEQ ID NO: 1067)
CAAgugaguc,
(SEQ ID NO: 2525)
UAGguagguc,
(SEQ ID NO: 2180)
GCGgugagcu,
(SEQ ID NO: 3143)
AUUgugagga,
(SEQ ID NO: 1361)
CAGgugcaca,
(SEQ ID NO: 3144)
CAGguuggaa,
(SEQ ID NO: 3145)
CUGgucacuu,
(SEQ ID NO: 2214)
GGAguaagug,
(SEQ ID NO: 2059)
GAGgugggcu,
(SEQ ID NO: 201)
AAGguacuug,
(SEQ ID NO: 3146)
AGGguaggau,
(SEQ ID NO: 3147)
AAUguguguu,
(SEQ ID NO: 568)
ACAguuaagu,
(SEQ ID NO: 2078)
GAGgugugug,
(SEQ ID NO: 3148)
AAGggggcu,
(SEQ ID NO: 3149)
AUAgcaagua,
(SEQ ID NO: 454)
AAGguuguua,
(SEQ ID NO: 3150)
CAAgcaaggc,
(SEQ ID NO: 3151)
GUGguaauua,
(SEQ ID NO: 3152)
UCUguucagu,
(SEQ ID NO: 754)
AGGguaggcc,
(SEQ ID NO: 3153)
AAGguaucau,
(SEQ ID NO: 2509)
UAGguaccuu,
(SEQ ID NO: 254)
AAGguaugac,
(SEQ ID NO: 2219)
GGAguaggua,
(SEQ ID NO: 3154)
UAAguuggca,
(SEQ ID NO: 3155)
AGUgugaggc,
(SEQ ID NO: 3156)
GAGguuugug,
(SEQ ID NO: 3157)
UGGgucugcu,
(SEQ ID NO: 1350)
CAGgugaucc,
(SEQ ID NO: 1283)
CAGgucagug,
(SEQ ID NO: 151)
AAGguaaggg,
(SEQ ID NO: 3158)
CAGgugcagu,
(SEQ ID NO: 2064)
GAGguggguc,
(SEQ ID NO: 2206)
GCUgugagug,
(SEQ ID NO: 3159)
AAGguggagu,
(SEQ ID NO: 3160)
GGGgucaguu,
(SEQ ID NO: 719)
AGCguaagug,
(SEQ ID NO: 691)
AGAguaugaa,
(SEQ ID NO: 3161)
GGGguagggu,
(SEQ ID NO: 3162)
AAGgccagca,
(SEQ ID NO: 3163)
CGAguaugcc,
(SEQ ID NO: 3164)
GUGgugagcg,
(SEQ ID NO: 481)
AAUguaaauu,
(SEQ ID NO: 1375)
CAGgugcgca,
(SEQ ID NO: 3165)
GGUguaugaa,
(SEQ ID NO: 1804)
CUUgugaguu,
(SEQ ID NO: 250)
AAGguaucuc,
(SEQ ID NO: 665)
AGAguaagga,
(SEQ ID NO: 2482)
UAGguaagac,
(SEQ ID NO: 2026)
GAGgugagug,
(SEQ ID NO: 1443)
CAGguguguu,
(SEQ ID NO: 3004)
UUGgugagua,
(SEQ ID NO: 3166)
AGGgcgaguu,
(SEQ ID NO: 3167)
CAGguuuugc,
(SEQ ID NO: 3168)
UUUgugaguu,
(SEQ ID NO: 736)
AGGguaagca,
(SEQ ID NO: 3169)
GAGguccucu,
(SEQ ID NO: 3170)
CCAgcaggua,
(SEQ ID NO: 3171)
GAGguucgcg,
(SEQ ID NO: 1351)
CAGgugaucu,
(SEQ ID NO: 625)
ACUguaagua,
(SEQ ID NO: 131)
AAGguaaauc,
(SEQ ID NO: 3172)
CAGgcaaaua,
(SEQ ID NO: 2346)
GUGguaagca,
(SEQ ID NO: 3173)
CAGguuaaau,
(SEQ ID NO: 3174)
UUGguaauaa,
(SEQ ID NO: 3175)
UAUguaggua,
(SEQ ID NO: 1225)
CAGguaguau,
(SEQ ID NO: 3176)
AAGgugugcc,
(SEQ ID NO: 2834)
UGGguaagag,
(SEQ ID NO: 3177)
CAGgcaagca,
(SEQ ID NO: 2961)
UUGguaaggg,
(SEQ ID NO: 109)
AAGgcaggug,
(SEQ ID NO: 3178)
ACGguaaaug,
(SEQ ID NO: 3179)
GCUgugagca,
(SEQ ID NO: 3180)
AUGguacaca,
(SEQ ID NO: 3181)
GUAguguguu,
(SEQ ID NO: 3182)
ACUguaagag,
(SEQ ID NO: 3183)
CCCgcagguc,
(SEQ ID NO: 2019)
GAGgugagcc,
(SEQ ID NO: 3184)
GAGgugcugu,
(SEQ ID NO: 3185)
UAAguaugcu,
(SEQ ID NO: 3186)
GAGgccaucu,
(SEQ ID NO: 2700)
UCAgugagug,
(SEQ ID NO: 3187)
CAGgugcuac,
(SEQ ID NO: 533)
AAUgugggug,
(SEQ ID NO: 3188)
GAGgugugaa,
(SEQ ID NO: 1730)
CUGguagguc,
(SEQ ID NO: 3189)
GUGgcgcgcg,
(SEQ ID NO: 1359)
CAGgugcaaa,
(SEQ ID NO: 3190)
UAAguggagg,
(SEQ ID NO: 3191)
CAUgugggua,
(SEQ ID NO: 3192)
GAGguagggu,
(SEQ ID NO: 61)
AAAgugaguu,
(SEQ ID NO: 3193)
AGGguucuag,
(SEQ ID NO: 3194)
UGUgugagcu,
(SEQ ID NO: 3195)
AGGgugaauc,
(SEQ ID NO: 3196)
CAGgucaggg,
(SEQ ID NO: 3197)
AAGgucccug,
(SEQ ID NO: 3198)
CUGguagagu,
(SEQ ID NO: 2570)
UAGgucaguu,
(SEQ ID NO: 19)
AAAguaaggg,
(SEQ ID NO: 1052)
CAAguaugug,
(SEQ ID NO: 3199)
CAGgugcuuu,
(SEQ ID NO: 169)
AAGguaauuc,
(SEQ ID NO: 3200)
GGGgugcacg,
(SEQ ID NO: 3201)
ACUgugcuac,
(SEQ ID NO: 3202)
CAGguaccua,
(SEQ ID NO: 1211)
CAGguagcuu,
(SEQ ID NO: 2873)
UGGgugaggc,
(SEQ ID NO: 1718)
CUGguacauu,
(SEQ ID NO: 3203)
AGGguaaucu,
(SEQ ID NO: 1161)
CAGguacaag,
(SEQ ID NO: 1157)
CAGguaauuc,
(SEQ ID NO: 3204)
AGGgcacuug,
(SEQ ID NO: 2587)
UAGgugagaa,
(SEQ ID NO: 3205)
GAGguaaugc,
(SEQ ID NO: 3206)
CCAgugaguu,
(SEQ ID NO: 44)
AAAguaugug,
(SEQ ID NO: 3207)
CUGgugaauc,
(SEQ ID NO: 2663)
UAUguaugua,
(SEQ ID NO: 3208)
CCUgcaggug,
(SEQ ID NO: 1245)
CAGguaucug,
(SEQ ID NO: 3209)
GAGgugaggu,
(SEQ ID NO: 3210)
CUGguaaaac,
(SEQ ID NO: 3211)
UGUgugugcu,
(SEQ ID NO: 3212)
CAGguuaagu,
(SEQ ID NO: 1152)
CAGguaaucc,
(SEQ ID NO: 3213)
UAGguauuug,
(SEQ ID NO: 2852)
UGGguagguc,
(SEQ ID NO: 1129)
CAGguaacag,
(SEQ ID NO: 3214)
AGCgugcgug,
(SEQ ID NO: 289)
AAGgucagga,
(SEQ ID NO: 2312)
GGUgugagcc,
(SEQ ID NO: 1707)
CUGguaagua,
(SEQ ID NO: 3215)
GGGgugggca,
(SEQ ID NO: 376)
AAGgugggaa,
(SEQ ID NO: 1347)
CAGgugagug,
(SEQ ID NO: 3216)
CUGguuguua,
(SEQ ID NO: 3217)
CAGguaauag,
(SEQ ID NO: 3218)
UAGgugaguu,
(SEQ ID NO: 671)
AGAguaaguu,
(SEQ ID NO: 3219)
UAGguaaucc,
(SEQ ID NO: 3220)
CCGgugacug,
(SEQ ID NO: 3221)
GUCgugauua,
(SEQ ID NO: 1794)
CUUguaagug,
(SEQ ID NO: 3222)
UAGguaguca,
(SEQ ID NO: 3223)
CUGguaaguc,
(SEQ ID NO: 3224)
AGGgugagcg,
(SEQ ID NO: 1255)
CAGguaugga,
(SEQ ID NO: 3225)
AUUgugacca,
(SEQ ID NO: 2411)
GUUgugggua,
(SEQ ID NO: 173)
AAGguacaag,
(SEQ ID NO: 3226)
CUAgcaagug,
(SEQ ID NO: 3227)
CUGgugagau,
(SEQ ID NO: 1406)
CAGgugggca,
(SEQ ID NO: 3228)
AUGgcucgag,
(SEQ ID NO: 1720)
CUGguacguu,
(SEQ ID NO: 3229)
UUGgugugua,
(SEQ ID NO: 3230)
GAGgugucug,
(SEQ ID NO: 3231)
GAGgugggac,
(SEQ ID NO: 3232)
GGGgugggag,
(SEQ ID NO: 3233)
GCAgcgugag,
(SEQ ID NO: 1870)
GAGguaaaga,
(SEQ ID NO: 1965)
GAGguaugua,
(SEQ ID NO: 336)
AAGgugagac,
(SEQ ID NO: 174)
AAGguacaau,
(SEQ ID NO: 3234)
CUGguaugag,
(SEQ ID NO: 3235)
AACguaaaau,
(SEQ ID NO: 2364)
GUGguaggga,
(SEQ ID NO: 1737)
CUGguaugug,
(SEQ ID NO: 3236)
CUUguaagca,
(SEQ ID NO: 223)
AAGguaggga,
(SEQ ID NO: 3237)
AUUguaagcc,
(SEQ ID NO: 895)
AUGguaagcu,
(SEQ ID NO: 1322)
CAGgugaauu,
(SEQ ID NO: 2581)
UAGgugaaua,
(SEQ ID NO: 3238)
CAAguaugga,
(SEQ ID NO: 936)
AUGguauggc,
(SEQ ID NO: 3239)
GAGgucaugc,
(SEQ ID NO: 1174)
CAGguacccu,
(SEQ ID NO: 3240)
ACAgugagac,
(SEQ ID NO: 3241)
CAGgucugau,
(SEQ ID NO: 3242)
GAAguugggu,
(SEQ ID NO: 1767)
CUGgugcgug,
(SEQ ID NO: 1180)
CAGguacgag,
(SEQ ID NO: 556)
ACAgugagcc,
(SEQ ID NO: 153)
AAGguaagua,
(SEQ ID NO: 3243)
GGAguaaggc,
(SEQ ID NO: 2077)
GAGgugugua,
(SEQ ID NO: 3244)
AAGgucauuu,
(SEQ ID NO: 3245)
CAGguagucu,
(SEQ ID NO: 3246)
AUGguaucug,
(SEQ ID NO: 125)
AAGguaaacu,
(SEQ ID NO: 1938)
GAGguaggug,
(SEQ ID NO: 1700)
CUGguaagca,
(SEQ ID NO: 734)
AGGguaagag,
(SEQ ID NO: 3247)
AAAguaaagc,
(SEQ ID NO: 1502)
CAGguuugag,
(SEQ ID NO: 3248)
GAGgcgggua,
(SEQ ID NO: 3249)
CGAguacgau
(SEQ ID NO: 1495)
CAGguuguug,
(SEQ ID NO: 3250)
AAAguauggg,
(SEQ ID NO: 3251)
UAGgcugguc,
(SEQ ID NO: 149)
AAGguaagga,
(SEQ ID NO: 458)
AAGguuuccu,
(SEQ ID NO: 3252)
UUGguaaaac,
(SEQ ID NO: 1893)
GAGguaagua,
(SEQ ID NO: 1465)
CAGguucaag,
(SEQ ID NO: 3253)
UGGguuaugu,
(SEQ ID NO: 2027)
GAGgugaguu,
(SEQ ID NO: 598)
ACGgugaaac,
(SEQ ID NO: 3254)
GAUguaacca,
(SEQ ID NO: 3255)
AAGgugcggg,
(SEQ ID NO: 3256)
CCGguacgug,
(SEQ ID NO: 3257)
GAUgugagaa,
(SEQ ID NO: 3258)
GUGgcgguga,
(SEQ ID NO: 3259)
CAGguauuag,
(SEQ ID NO: 3260)
GAGguuggga,
(SEQ ID NO: 3261)
AAGgcuagua,
(SEQ ID NO: 381)
AAGgugggg,
(SEQ ID NO: 3262)
CAGgcaggga,
(SEQ ID NO: 3263)
AAUguuaguu,
(SEQ ID NO: 3264)
GAGguaaagg,
(SEQ ID NO: 1437)
CAGgugugcu,
(SEQ ID NO: 1733)
CUGguaugau,
(SEQ ID NO: 978)
AUGguuaguc,
(SEQ ID NO: 1751)
CUGgugagaa,
(SEQ ID NO: 3265)
CAGgccggcg,
(SEQ ID NO: 1332)
CAGgugacug,
(SEQ ID NO: 20)
AAAguaaggu,
(SEQ ID NO: 3266)
UAAguacuug,
(SEQ ID NO: 127)
AAGguaaagc,
(SEQ ID NO: 3267)
UCGguagggg,
(SEQ ID NO: 1212)
CAGguaggaa,
(SEQ ID NO: 817)
AGUguaagca,
(SEQ ID NO: 3268)
CCCgugagau,
(SEQ ID NO: 3269)
GUGguuguuu,
(SEQ ID NO: 1504)
CAGguuugcc,
(SEQ ID NO: 766)
AGGguauggg,
(SEQ ID NO: 2424)
UAAguaagug,
(SEQ ID NO: 3270)
GAGguaagac,
(SEQ ID NO: 3271)
GAUguagguc,
(SEQ ID NO: 1043)
CAAguaggug,
(SEQ ID NO: 845)
AUAguaaaua,
(SEQ ID NO: 3272)
GAGguugggg,
(SEQ ID NO: 3273)
GAGgcgagua,
(SEQ ID NO: 1229)
CAGguagugu,
(SEQ ID NO: 2366)
GUGguaggug,
(SEQ ID NO: 1068)
CAAgugagug,
(SEQ ID NO: 330)
AAGgugacaa,
(SEQ ID NO: 3274)
CCAgcguaau,
(SEQ ID NO: 3275)
ACGgugaggu,
(SEQ ID NO: 3276)
GGGguauauu,
(SEQ ID NO: 1345)
CAGgugagua,
(SEQ ID NO: 364)
AAGgugcgug,
(SEQ ID NO: 3277)
UAUguaaauu,
(SEQ ID NO: 1281)
CAGgucagua,
(SEQ ID NO: 3278)
ACGguacuua,
(SEQ ID NO: 3279)
GAGgucagca,
(SEQ ID NO: 2431)
UAAguaugua,
(SEQ ID NO: 3280)
GGGgucagac,
(SEQ ID NO: 3281)
AAUgugugag,
(SEQ ID NO: 3282)
UCCgucagua,
(SEQ ID NO: 1391)
CAGgugcuuc,
(SEQ ID NO: 3283)
CCAguuagug,
(SEQ ID NO: 1590)
CCGgugggcg,
(SEQ ID NO: 3284)
AGGgugcaug,
(SEQ ID NO: 3285)
GGGguaggau,
(SEQ ID NO: 2615)
UAGgugggcc,
(SEQ ID NO: 3286)
GAGguguucg,
(SEQ ID NO: 3287)
UUGgcaagaa,
(SEQ ID NO: 3288)
UCCguaagua,
(SEQ ID NO: 3289)
CAGguguaag,
(SEQ ID NO: 1680)
CUCgugagua,
(SEQ ID NO: 3290)
GAGguguuuu,
(SEQ ID NO: 2018)
GAGgugagca,
(SEQ ID NO: 1872)
GAGguaaagu,
(SEQ ID NO: 193)
AAGguacguu,
(SEQ ID NO: 1291)
CAGguccagu,
(SEQ ID NO: 947)
AUGgugaaac,
(SEQ ID NO: 3291)
GUAgugagcu,
(SEQ ID NO: 3292)
CAGgugaaaa,
(SEQ ID NO: 3293)
AGGguacagg,
(SEQ ID NO: 3294)
AAGguaacgc,
(SEQ ID NO: 3295)
AAGguauacc,
(SEQ ID NO: 3296)
CCUgugagau,
(SEQ ID NO: 3297)
GGGguacgug,
(SEQ ID NO: 1964)
GAGguauggu,
(SEQ ID NO: 2557)
UAGguauuau,
(SEQ ID NO: 3298)
GAAguaggag,
(SEQ ID NO: 3299)
UCGguaaggg,
(SEQ ID NO: 3300)
CCGguaagcg,
(SEQ ID NO: 1823)
GAAguaauua,
(SEQ ID NO: 1346)
CAGgugaguc,
(SEQ ID NO: 279)
AAGgucaaga,
(SEQ ID NO: 899)
AUGguaaguc,
(SEQ ID NO: 1340)
CAGgugagcu,
(SEQ ID NO: 3301)
CCAguuuuug,
(SEQ ID NO: 1404)
CAGgugggag,
(SEQ ID NO: 270)
AAGguauuau,
(SEQ ID NO: 130)
AAGguaaaua,
(SEQ ID NO: 3302)
AAGgugcugu,
(SEQ ID NO: 3303)
AAAguacacc,
(SEQ ID NO: 1783)
CUGguucgug,
(SEQ ID NO: 2690)
UCAguaaguc,
(SEQ ID NO: 3304)
GAAguacgug,
(SEQ ID NO: 1323)
CAGgugacaa,
(SEQ ID NO: 2832)
UGGguaagaa,
(SEQ ID NO: 3305)
UGUguagggg,
(SEQ ID NO: 1932)
GAGguaggca,
(SEQ ID NO: 3306)
UUGgugaggc,
(SEQ ID NO: 974)
AUGgugugua,
(SEQ ID NO: 3307)
CAGguccucc,
(SEQ ID NO: 2953)
UUGguaaaug,
(SEQ ID NO: 2207)
GCUgugaguu,
(SEQ ID NO: 3308)
AUGgucugua,
(SEQ ID NO: 3309)
CAUgcaggug,
(SEQ ID NO: 3310)
CUGguacacc,
(SEQ ID NO: 3311)
CAGguccuua,
(SEQ ID NO: 1031)
CAAguaaucu,
(SEQ ID NO: 3312)
AUGgcagccu,
(SEQ ID NO: 282)
AAGgucagaa,
(SEQ ID NO: 3313)
AACgugaggc,
(SEQ ID NO: 1106)
CAGgcacgca,
(SEQ ID NO: 3314)
ACGguccagg,
(SEQ ID NO: 3315)
UCUguacaua,
(SEQ ID NO: 3316)
GAGgugauua,
(SEQ ID NO: 3317)
ACGguaaaua,
(SEQ ID NO: 3318)
AUGguaacug,
(SEQ ID NO: 3319)
CAGgcgcguu,
(SEQ ID NO: 1235)
CAGguauaga,
(SEQ ID NO: 3320)
AAGguuuguu,
(SEQ ID NO: 1247)
CAGguaugaa,
(SEQ ID NO: 2638)
UAGguuggua,
(SEQ ID NO: 1752)
CUGgugagac,
(SEQ ID NO: 3321)
CAGguuagga,
(SEQ ID NO: 3322)
AUGgugacug,
(SEQ ID NO: 3323)
UUGguauccc,
(SEQ ID NO: 3324)
CUUguaggac,
(SEQ ID NO: 69)
AAAguguguu,
(SEQ ID NO: 1500)
CAGguuucuu,
(SEQ ID NO: 3325)
GGGguauggc,
(SEQ ID NO: 3326)
GGGguaggac,
(SEQ ID NO: 626)
ACUguaaguc,
(SEQ ID NO: 3327)
AUCguaagcu,
(SEQ ID NO: 2636)
UAGguucccc,
(SEQ ID NO: 3328)
GGUgugagca,
(SEQ ID NO: 3329)
CUGguuggua,
(SEQ ID NO: 3330)
GGGguuaggg,
(SEQ ID NO: 3331)
UGAguaagaa,
(SEQ ID NO: 1969)
GAGguauucc,
(SEQ ID NO: 2893)
UGGguuaguc,
(SEQ ID NO: 3332)
CAGgcucgug,
(SEQ ID NO: 3333)
UAGguagagu,
(SEQ ID NO: 3334)
UAGgugcccu,
(SEQ ID NO: 58)
AAAgugagua,
(SEQ ID NO: 2094)
GAGguucaua,
(SEQ ID NO: 2958)
UUGguaagag,
(SEQ ID NO: 3335)
ACCgugugua,
(SEQ ID NO: 3336)
UAUguaguau,
(SEQ ID NO: 3337)
UGGguaauag,
(SEQ ID NO: 3338)
CAGgucugaa,
(SEQ ID NO: 3339)
AAAguauaaa,
(SEQ ID NO: 3340)
GUGgugaguc,
(SEQ ID NO: 3341)
AGUgugauua,
(SEQ ID NO: 3020)
UUGgugugug,
(SEQ ID NO: 1353)
CAGgugaugg,
(SEQ ID NO: 2204)
GCUgugagua,
(SEQ ID NO: 1169)
CAGguacaug,
(SEQ ID NO: 178)
AAGguacagu,
(SEQ ID NO: 3342)
GAAguuguag,
(SEQ ID NO: 1355)
CAGgugauua,
(SEQ ID NO: 2583)
UAGgugaauu,
(SEQ ID NO: 3343)
GGUguuaaua,
(SEQ ID NO: 1268)
CAGguauuua,
(SEQ ID NO: 3344)
CAAguacucg,
(SEQ ID NO: 1022)
CAAguaagaa,
(SEQ ID NO: 188)
AAGguaccuu,
(SEQ ID NO: 3345)
ACGgugaggg,
(SEQ ID NO: 3346)
UGAgcaggca,
(SEQ ID NO: 3347)
GGGgugaccg,
(SEQ ID NO: 1875)
GAGguaaaug,
(SEQ ID NO: 3348)
CGGguuugug,
(SEQ ID NO: 341)
AAGgugagcg,
(SEQ ID NO: 3349)
GUGguaugga,
(SEQ ID NO: 1703)
CUGguaagga,
(SEQ ID NO: 1911)
GAGguaccag,
(SEQ ID NO: 1587)
CCGgugagug,
(SEQ ID NO: 416)
AAGguuagaa,
(SEQ ID NO: 1921)
GAGguacuug,
(SEQ ID NO: 651)
AGAguaaaac,
(SEQ ID NO: 2760)
UCUgugagua,
(SEQ ID NO: 3350)
AAGgcgggaa,
(SEQ ID NO: 1253)
CAGguaugcg,
(SEQ ID NO: 3351)
AGGguaaaac,
(SEQ ID NO: 333)
AAGgugacug,
(SEQ ID NO: 3352)
AGGguauguu,
(SEQ ID NO: 263)
AAGguaugua,
(SEQ ID NO: 1302)
CAGgucucuc,
(SEQ ID NO: 3353)
CAGgcaugua,
(SEQ ID NO: 1729)
CUGguaggua,
(SEQ ID NO: 3354)
AAGgucaugc,
(SEQ ID NO: 1163)
CAGguacaca,
(SEQ ID NO: 3355)
GAUguacguu,
(SEQ ID NO: 3356)
ACAguacgug,
(SEQ ID NO: 3357)
ACGguaccca,
(SEQ ID NO: 3358)
CAGguagugc,
(SEQ ID NO: 3359)
ACAguaagag,
(SEQ ID NO: 3360)
GGUgcacacc,
(SEQ ID NO: 3361)
GAGguguaac,
(SEQ ID NO: 403)
AAGgugugua,
(SEQ ID NO: 3362)
UAGguacuua,
(SEQ ID NO: 3363)
GCGguacugc,
(SEQ ID NO: 2842)
UGGguaaguc,
(SEQ ID NO: 1529)
CAUguaggua,
(SEQ ID NO: 3364)
CAGguaggau,
(SEQ ID NO: 3365)
CAGgucuggc,
(SEQ ID NO: 3366)
GUGguuuuaa,
(SEQ ID NO: 1402)
CAGgugggaa,
(SEQ ID NO: 2875)
UGGgugagua,
(SEQ ID NO: 3367)
CGAgugagcc,
(SEQ ID NO: 261)
AAGguauggc,
(SEQ ID NO: 3368)
AGUguuguca,
(SEQ ID NO: 1358)
CAGgugauuu,
(SEQ ID NO: 2544)
UAGguaucuc,
(SEQ ID NO: 3369)
UAAguauguu,
(SEQ ID NO: 3370)
AAGguugagc,
(SEQ ID NO: 653)
AGAguaaaga,
(SEQ ID NO: 3371)
GGUguaagua,
(SEQ ID NO: 2279)
GGGgugagcu,
(SEQ ID NO: 3372)
CAGguauaau,
(SEQ ID NO: 1904)
GAGguacaaa,
(SEQ ID NO: 3373)
AUGguaccaa,
(SEQ ID NO: 2523)
UAGguagggg,
(SEQ ID NO: 3374)
UGAgucagaa,
(SEQ ID NO: 3375)
AAGgcaauua,
(SEQ ID NO: 3376)
UUGguaagau,
(SEQ ID NO: 1165)
CAGguacaga,
(SEQ ID NO: 3377)
AGAguuagag,
(SEQ ID NO: 1381)
CAGgugcguc,
(SEQ ID NO: 3378)
GAGguauuac,
(SEQ ID NO: 3379)
ACGguacaga,
(SEQ ID NO: 1313)
CAGgucuucc,
(SEQ ID NO: 152)
AAGguaaggu,
(SEQ ID NO: 1903)
GAGguaauuu,
(SEQ ID NO: 3380)
AGUguaggcu,
(SEQ ID NO: 3381)
AAAguaagcg,
(SEQ ID NO: 3382)
CCUguaagcc,
(SEQ ID NO: 3383)
AGGgugauuu,
(SEQ ID NO: 3384)
UGUguaugaa,
(SEQ ID NO: 3385)
CUGguacaca,
(SEQ ID NO: 3386)
AGGguagaga,
(SEQ ID NO: 848)
AUAguaagca,
(SEQ ID NO: 3387)
AGAguaugua,
(SEQ ID NO: 3388)
UUGgucagca,
(SEQ ID NO: 1105)
CAGgcaaguu,
(SEQ ID NO: 242)
AAGguauaua,
(SEQ ID NO: 314)
AAGgucugga,
(SEQ ID NO: 1181)
CAGguacgca,
(SEQ ID NO: 3389)
AGGgugcggg,
(SEQ ID NO: 900)
AUGguaagug,
(SEQ ID NO: 3390)
AAAgugauga,
(SEQ ID NO: 3391)
UGCgugagua,
(SEQ ID NO: 684)
AGAguaggga,
(SEQ ID NO: 2912)
UGUguaggua,
(SEQ ID NO: 2521)
UAGguaggau,
(SEQ ID NO: 2440)
UAAgugagug,
(SEQ ID NO: 2193)
GCUguaagua,
(SEQ ID NO: 1814)
GAAguaagaa,
(SEQ ID NO: 2733)
UCGgugaggc,
(SEQ ID NO: 2564)
UAGguauuuu,
(SEQ ID NO: 3392)
AAGguacaca,
(SEQ ID NO: 227)
AAGguaggua,
(SEQ ID NO: 2854)
UGGguagguu,
(SEQ ID NO: 541)
ACAgcaagua,
(SEQ ID NO: 1931)
GAGguaggag,
(SEQ ID NO: 2878)
UGGgugaguu,
(SEQ ID NO: 3393)
GCGgugagau,
(SEQ ID NO: 3394)
CCUguagguu,
(SEQ ID NO: 1440)
CAGgugugua,
(SEQ ID NO: 1701)
CUGguaagcc,
(SEQ ID NO: 3395)
AAGgugauuc,
(SEQ ID NO: 1208)
CAGguagcua,
(SEQ ID NO: 3396)
GUUguaagug,
(SEQ ID NO: 893)
AUGguaagca,
(SEQ ID NO: 3397)
AUAguaggga,
(SEQ ID NO: 3398)
GGGguucgcu,
(SEQ ID NO: 3399)
CCGgucagag,
(SEQ ID NO: 3400)
GUAguaugag,
(SEQ ID NO: 3401)
CGUguaagau,
(SEQ ID NO: 3402)
UGAguaggca,
(SEQ ID NO: 3403)
UCAguaugua,
(SEQ ID NO: 1954)
GAGguaucug,
(SEQ ID NO: 3404)
AGAguauuuu,
(SEQ ID NO: 3405)
AAGguuguag,
(SEQ ID NO: 821)
AGUguaaguu,
(SEQ ID NO: 1626)
CGGguaaguu,
(SEQ ID NO: 3406)
UCGgugcgga,
(SEQ ID NO: 2491)
UAGguaagua,
(SEQ ID NO: 3407)
GAAguuagau,
(SEQ ID NO: 3408)
GCUgugagac,
(SEQ ID NO: 3409)
CAGgcaggua,
(SEQ ID NO: 1218)
CAGguagggg,
(SEQ ID NO: 3410)
UAAguuaaga,
(SEQ ID NO: 970)
AUGguggguu,
(SEQ ID NO: 2494)
UAGguaaguu,
(SEQ ID NO: 1690)
CUGguaaauu,
(SEQ ID NO: 1577)
CCGguaagga,
(SEQ ID NO: 3411)
GAGgcaggca,
(SEQ ID NO: 1523)
CAUguaagug,
(SEQ ID NO: 3412)
AAGgugccua,
(SEQ ID NO: 2977)
UUGguaggga,
(SEQ ID NO: 123)
AAGguaaaca,
(SEQ ID NO: 3413)
CGGgugugag,
(SEQ ID NO: 3414)
GGGgugugag,
(SEQ ID NO: 3415)
UCCguggguc,
(SEQ ID NO: 3416)
ACGguaaauc,
(SEQ ID NO: 3417)
UCAguaggua,
(SEQ ID NO: 1278)
CAGgucagcc,
(SEQ ID NO: 3418)
CAGgcggugg,
(SEQ ID NO: 3419)
CGAguaagcu,
(SEQ ID NO: 3420)
CCCgugagca,
(SEQ ID NO: 3421)
AAAguaauga,
(SEQ ID NO: 1702)
CUGguaagcu,
(SEQ ID NO: 3422)
CGGguaacca,
(SEQ ID NO: 3423)
CAGgucgcac,
(SEQ ID NO: 3424)
GAGguaggcc,
(SEQ ID NO: 2591)
UAGgugagcc,
(SEQ ID NO: 3425)
UAGguaggca,
(SEQ ID NO: 3426)
GCGgugcgug,
(SEQ ID NO: 961)
AUGgugagua,
(SEQ ID NO: 2282)
GGGgugaggg,
(SEQ ID NO: 3427)
GAGgucacac,
(SEQ ID NO: 3428)
CAGguaggcc,
(SEQ ID NO: 3429)
CAAgugcuga,
(SEQ ID NO: 3430)
GUCgucuuca,
(SEQ ID NO: 1518)
CAUguaagaa,
(SEQ ID NO: 3431)
GUAguaagga,
(SEQ ID NO: 2643)
UAGguuugua,
(SEQ ID NO: 3432)
CAAguuagag,
(SEQ ID NO: 208)
AAGguagagu,
(SEQ ID NO: 338)
AAGgugagau,
(SEQ ID NO: 37)
AAAguaggua,
(SEQ ID NO: 3433)
ACAgugaauc,
(SEQ ID NO: 1436)
CAGgugugcg,
(SEQ ID NO: 1299)
CAGgucggcc,
(SEQ ID NO: 3434)
AAGguaguau,
(SEQ ID NO: 3435)
ACUgucaguc,
(SEQ ID NO: 3436)
UCUgcagccu,
(SEQ ID NO: 3437)
CGAguaagug,
(SEQ ID NO: 3438)
AGAguaauua,
(SEQ ID NO: 837)
AGUgugagug,
(SEQ ID NO: 3439)
CCGgugagcg,
(SEQ ID NO: 3440)
AAGguaaccu,
(SEQ ID NO: 3441)
AAGguugugg,
(SEQ ID NO: 3442)
AAGgcauggg,
(SEQ ID NO: 284)
AAGgucagag,
(SEQ ID NO: 3443),
ACGguaaggu
(SEQ ID NO: 3444)
GGGgugagca,
(SEQ ID NO: 3445)
GAGguugcuu,
(SEQ ID NO: 3446)
AAGguaucgc,
(SEQ ID NO: 3447)
CCGguaaagg,
(SEQ ID NO: 3448)
AAAguuaaug,
(SEQ ID NO: 2510)
UAGguacgag,
(SEQ ID NO: 3449)
ACCguaauua,
(SEQ ID NO: 2249)
GGGguaagga,
(SEQ ID NO: 3450)
CCGguaacgc,
(SEQ ID NO: 1275)
CAGgucagaa,
(SEQ ID NO: 197)
AAGguacuga,
(SEQ ID NO: 2010)
GAGgugacca,
(SEQ ID NO: 2277)
GGGgugagcc,
(SEQ ID NO: 177)
AAGguacagg,
(SEQ ID NO: 3451)
AUGguaauua,
(SEQ ID NO: 1335)
CAGgugagag,
(SEQ ID NO: 3452)
AAGgugacuc,
(SEQ ID NO: 849)
AUAguaagua,
(SEQ ID NO: 1869)
GAGguaaacc,
(SEQ ID NO: 1405)
CAGgugggau,
(SEQ ID NO: 1333)
CAGgugagaa,
(SEQ ID NO: 3453)
AGGguaaaaa,
(SEQ ID NO: 3454)
GAGgugugac,
(SEQ ID NO: 3455)
CACguaagcu,
(SEQ ID NO: 3456)
CAGguccccc,
(SEQ ID NO: 3457)
CAGgucaggu,
(SEQ ID NO: 3458)
CGGguaaguc,
(SEQ ID NO: 3459)
ACGguauggg,
(SEQ ID NO: 2123)
GAUguaaguu,
(SEQ ID NO: 3460)
CAAguaauau,
(SEQ ID NO: 3461)
CAGguugggg,
(SEQ ID NO: 3462)
CCUgugcugg,
(SEQ ID NO: 256)
AAGguaugau,
(SEQ ID NO: 3463)
AGGguagagg,
(SEQ ID NO: 386)
AAGguggguu,
(SEQ ID NO: 1430)
CAGgugugaa,
(SEQ ID NO: 2988)
UUGguaugug,
(SEQ ID NO: 2985)
UUGguaucuc,
(SEQ ID NO: 2284)
GGGgugagug,
(SEQ ID NO: 1779)
CUGgugugug,
(SEQ ID NO: 3464)
AGGguagggc,
(SEQ ID NO: 3465)
GUGgugagua,
(SEQ ID NO: 1258)
CAGguaugua,
(SEQ ID NO: 181)
AAGguacauu,
(SEQ ID NO: 2934)
UUAguaagug,
(SEQ ID NO: 3466)
AAUguauauc,
(SEQ ID NO: 1793)
CUUguaagua,
(SEQ ID NO: 2087)
GAGguuagua,
(SEQ ID NO: 1146)
CAGguaaggu,
(SEQ ID NO: 1155)
CAGguaaugu,
(SEQ ID NO: 3467)
AGGgugaggc,
(SEQ ID NO: 1269)
CAGguauuuc,
(SEQ ID NO: 1307)
CAGgucugga,
(SEQ ID NO: 3468)
GGGgugugcu,
(SEQ ID NO: 2598)
UAGgugagug,
(SEQ ID NO: 3469)
AAUguaaccu,
(SEQ ID NO: 2439)
UAAgugaguc,
(SEQ ID NO: 3470)
CAGgugcacu,
(SEQ ID NO: 579)
ACGguaagua,
(SEQ ID NO: 3471)
GAGguauccu,
(SEQ ID NO: 2745)
UCUguaaguc,
(SEQ ID NO: 1263)
CAGguauuca,
(SEQ ID NO: 3472)
UGUguaagug,
(SEQ ID NO: 3473)
CCAgcaaggc,
(SEQ ID NO: 2006)
GAGgugaagg,
(SEQ ID NO: 3474)
AAUguggggu,
(SEQ ID NO: 3475)
UCGgugcgug,
(SEQ ID NO: 3476)
UUGguaaggc,
(SEQ ID NO: 3477)
GAGguaagug,
(SEQ ID NO: 14)
AAAguaagau,
(SEQ ID NO: 3478)
UAGgucuuuu,
(SEQ ID NO: 3479)
GAGgucugau,
(SEQ ID NO: 3480)
CCAguuagag,
(SEQ ID NO: 3481)
UGGgugaaaa,
(SEQ ID NO: 662)
AGAguaagau,
(SEQ ID NO: 1158)
CAGguaauug,
(SEQ ID NO: 3482)
CAGgccgguc,
(SEQ ID NO: 3483)
CCGguaagag,
(SEQ ID NO: 2021)
GAGgugagcu,
(SEQ ID NO: 3484)
CUGguaagac,
(SEQ ID NO: 1336)
CAGgugagau,
(SEQ ID NO: 3485)
CUGguuuguu,
(SEQ ID NO: 3486)
UGGguaggua,
(SEQ ID NO: 1457)
CAGguuagug,
(SEQ ID NO: 3487)
CAGguguucg,
(SEQ ID NO: 3488)
CGGguagguc,
(SEQ ID NO: 3489)
GUGguacaua,
(SEQ ID NO: 194)
AAGguacuaa,
(SEQ ID NO: 3490)
GAUgugagua,
(SEQ ID NO: 2904)
UGUguaagac,
(SEQ ID NO: 3491)
GAGguagccg,
(SEQ ID NO: 3492)
UAGgugaucu,
(SEQ ID NO: 1185)
CAGguacgug,
(SEQ ID NO: 3493)
CUUgucaguc,
(SEQ ID NO: 3494)
GAGguaucac,
(SEQ ID NO: 3495)
GAGguaauga,
(SEQ ID NO: 3496)
AAGguaacac,
(SEQ ID NO: 1123)
CAGguaaagc,
(SEQ ID NO: 3497)
AAGgcaagua,
(SEQ ID NO: 3498)
CGCgugagcc,
(SEQ ID NO: 3499)
AGUgugcguu,
(SEQ ID NO: 2118)
GAUguaagca,
(SEQ ID NO: 159)
AAGguaauag,
(SEQ ID NO: 3500)
GGAgcaguug,
(SEQ ID NO: 3501)
AGCguaagau,
(SEQ ID NO: 290)
AAGgucaggc,
(SEQ ID NO: 3502)
GAGguauuca,
(SEQ ID NO: 3503)
AAUguaaagu,
(SEQ ID NO: 3504)
CAGguaacaa,
(SEQ ID NO: 3505)
UCGguaggug,
(SEQ ID NO: 22)
AAAguaaguc,
(SEQ ID NO: 3506)
CGGgugcagu,
(SEQ ID NO: 3507)
GGUgugugca,
(SEQ ID NO: 2794)
UGAgugagaa,
(SEQ ID NO: 3508)
CACguguaag,
(SEQ ID NO: 3509)
GUGguuggua,
(SEQ ID NO: 3510)
GCAgccuuga,
(SEQ ID NO: 3511)
CGAgugugau,
(SEQ ID NO: 3512)
CAGguauaua,
(SEQ ID NO: 2665)
UAUguaugug,
(SEQ ID NO: 3513)
CCCgugguca,
(SEQ ID NO: 890)
AUGguaagac,
(SEQ ID NO: 2074)
GAGgugugga,
(SEQ ID NO: 3514)
AGUguauccu,
(SEQ ID NO: 3515)
UGAguguguc,
(SEQ ID NO: 3516)
UGGguaaucu,
(SEQ ID NO: 3517)
AUGgcagguu,
(SEQ ID NO: 1884)
GAGguaagau,
(SEQ ID NO: 3518)
UCAgcagcgu,
(SEQ ID NO: 378)
AAGgugggau,
(SEQ ID NO: 3519)
CGGgugcgcu,
(SEQ ID NO: 1429)
CAGgugucug,
(SEQ ID NO: 3520)
AGCgugguaa,
(SEQ ID NO: 3521)
AAUgugaaug,
(SEQ ID NO: 3522)
UCGgugagac,
(SEQ ID NO: 3523)
UAGguaaagc,
(SEQ ID NO: 3524)
CUGguaaaag,
(SEQ ID NO: 3525)
CCGgugcgga,
(SEQ ID NO: 3526)
CAGguacuca,
(SEQ ID NO: 1203)
CAGguagcaa,
(SEQ ID NO: 3527)
GAAguugagu,
(SEQ ID NO: 2052)
GAGguggagg,
(SEQ ID NO: 762)
AGGguaugag,
(SEQ ID NO: 3528)
UAGguaugcu,
(SEQ ID NO: 2588)
UAGgugagac,
(SEQ ID NO: 1156)
CAGguaauua,
(SEQ ID NO: 3529)
CGUguaagcc,
(SEQ ID NO: 1795)
CUUguaaguu,
(SEQ ID NO: 140)
AAGguaacuu,
(SEQ ID NO: 3530)
UCGgcaaggc,
(SEQ ID NO: 3531)
GAGguucucg,
(SEQ ID NO: 2058)
GAGgugggcg,
(SEQ ID NO: 3532)
AAGgcaugug,
(SEQ ID NO: 1738)
CUGguauguu,
(SEQ ID NO: 3533)
UAAgucauuu,
(SEQ ID NO: 1525)
CAUguaauua,
(SEQ ID NO: 3534)
AAUguaaaga,
(SEQ ID NO: 3535)
UAGgugcuca,
(SEQ ID NO: 166)
AAGguaaugg,
(SEQ ID NO: 3536)
GAGguacuga,
(SEQ ID NO: 2841)
UGGguaagua,
(SEQ ID NO: 3537)
UGGguaaaaa,
(SEQ ID NO: 342)
AAGgugagcu,
(SEQ ID NO: 3538)
UACgugaguu,
(SEQ ID NO: 790)
AGGgugagcc,
(SEQ ID NO: 3539)
CGGgugagga,
(SEQ ID NO: 2869)
UGGgugagag,
(SEQ ID NO: 3540)
GGUguaagcu,
(SEQ ID NO: 1648)
CGGguggguu,
(SEQ ID NO: 3541)
CCAgcuaagu,
(SEQ ID NO: 467)
AAGguuuguc,
(SEQ ID NO: 2084)
GAGguuagac,
(SEQ ID NO: 3542)
GAGguaccuc,
(SEQ ID NO: 3041)
UUUguaaguu,
(SEQ ID NO: 3543)
GAGguuagga,
(SEQ ID NO: 1216)
CAGguaggga,
(SEQ ID NO: 744)
AGGguaauac,
(SEQ ID NO: 3544)
UGCgugugua,
(SEQ ID NO: 3545)
CCAguaacca,
(SEQ ID NO: 3546)
AGGgucuguc,
(SEQ ID NO: 2860)
UGGguaugua,
(SEQ ID NO: 2348)
GUGguaagcu,
(SEQ ID NO: 3547)
CAGguaaccu,
(SEQ ID NO: 350)
AAGgugaguu,
(SEQ ID NO: 3548)
UAGguucgug,
(SEQ ID NO: 3549)
AAAguuagua,
(SEQ ID NO: 2816)
UGGgcaaguc,
(SEQ ID NO: 3550)
AAGgcacagu,
(SEQ ID NO: 2401)
GUUguaaguc,
(SEQ ID NO: 462)
AAGguuugcc,
(SEQ ID NO: 3551)
CUUgcauggg,
(SEQ ID NO: 3552)
GCGgugagua,
(SEQ ID NO: 3553)
GGGguaagcg,
(SEQ ID NO: 3554)
GCCguaagaa,
(SEQ ID NO: 3555)
GAGgucggga,
(SEQ ID NO: 2990)
UUGguauugu,
(SEQ ID NO: 3556)
AGUgugagac,
(SEQ ID NO: 1770)
CUGgugggga,
(SEQ ID NO: 668)
AGAguaaggu,
(SEQ ID NO: 3557)
CCGguggguc,
(SEQ ID NO: 1264)
CAGguauucu,
(SEQ ID NO: 3558)
UGGguaacgu,
(SEQ ID NO: 3559)
UUGgugagag,
(SEQ ID NO: 3560)
UAGguacccu,
(SEQ ID NO: 3561)
GGGgugcguc,
(SEQ ID NO: 3562)
AAGgcaggag,
(SEQ ID NO: 3563)
ACGguacauu,
(SEQ ID NO: 1946)
GAGguaguua,
(SEQ ID NO: 1256)
CAGguauggg,
(SEQ ID NO: 3053)
UUUguguguc,
(SEQ ID NO: 1194)
CAGguacuua,
(SEQ ID NO: 3564)
AUGguauacu,
(SEQ ID NO: 833)
AGUgugagcc,
(SEQ ID NO: 3565)
ACAguaacga,
(SEQ ID NO: 3566)
CUGguaccca,
(SEQ ID NO: 3567)
CAGguaaccc,
(SEQ ID NO: 3568)
GGAguaagua,
(SEQ ID NO: 2065)
GAGgugggug,
(SEQ ID NO: 3569)
ACUguauguc,
(SEQ ID NO: 606)
ACGgugagua,
(SEQ ID NO: 3570)
CUGguaaugu,
(SEQ ID NO: 247)
AAGguaucag,
(SEQ ID NO: 1370)
CAGgugcccc,
(SEQ ID NO: 3571)
AGUgucagug,
(SEQ ID NO: 218)
AAGguaggag,
(SEQ ID NO: 3572)
GGAguaugug,
(SEQ ID NO: 2992)
UUGguauuuu,
(SEQ ID NO: 3573)
CCUguuguga,
(SEQ ID NO: 3033)
UUUguaagaa,
(SEQ ID NO: 2475)
UAGguaacau,
(SEQ ID NO: 3574)
CAGguaagca,
(SEQ ID NO: 3575)
CAGgucacag,
(SEQ ID NO: 1432)
CAGgugugag,
(SEQ ID NO: 3576)
UAGguuugcg,
(SEQ ID NO: 1697)
CUGguaagaa,
(SEQ ID NO: 3577)
ACGguuguau,
(SEQ ID NO: 446)
AAGguugggg,
(SEQ ID NO: 329)
AAGgugaauu,
(SEQ ID NO: 3578)
GGGguuaguu,
(SEQ ID NO: 3579)
ACGguaaggc,
(SEQ ID NO: 1496)
CAGguuuaag,
(SEQ ID NO: 1709)
CUGguaaguu,
(SEQ ID NO: 3580)
GGGgugagag,
(SEQ ID NO: 2886)
UGGguggguu,
(SEQ ID NO: 2111)
GAGguuuguu,
(SEQ ID NO: 2826)
UGGguaaaug,
(SEQ ID NO: 3581)
CAGgcaggcc,
(SEQ ID NO: 3582)
CACgugcagg,
(SEQ ID NO: 340)
AAGgugagcc,
(SEQ ID NO: 1028)
CAAguaagug,
(SEQ ID NO: 1282)
CAGgucaguc,
(SEQ ID NO: 3583)
GCGguauaau,
(SEQ ID NO: 3584)
UAGguaaagu,
(SEQ ID NO: 3585)
UAGguggauu,
(SEQ ID NO: 3586)
GAGgucugga,
(SEQ ID NO: 3587)
UCGgucaguu,
(SEQ ID NO: 3588)
UGGguaacug,
(SEQ ID NO: 3589)
AAGguuugau,
(SEQ ID NO: 3590)
UGUgcuggug,
(SEQ ID NO: 3591)
UGUguaccuc,
(SEQ ID NO: 2849)
UGGguacagu,
(SEQ ID NO: 3592)
AUCgucagcg,
(SEQ ID NO: 3593)
CAGgucuugg,
(SEQ ID NO: 3594)
GAAguuggua,
(SEQ ID NO: 3595)
GAAguaaaga,
(SEQ ID NO: 2959)
UUGguaagcu,
(SEQ ID NO: 2507)
UAGguaccag,
(SEQ ID NO: 3596)
AGGguaucau,
(SEQ ID NO: 1118)
CAGguaaaaa,
(SEQ ID NO: 583)
ACGguaauuu,
(SEQ ID NO: 997)
AUUguaaguu,
(SEQ ID NO: 1908)
GAGguacagu,
(SEQ ID NO: 1315)
CAGgugaaag,
(SEQ ID NO: 3597)
UGGguuguuu,
(SEQ ID NO: 2259)
GGGguaggug,
(SEQ ID NO: 1369)
CAGgugccca,
(SEQ ID NO: 3598)
AGCgugagau,
(SEQ ID NO: 1565)
CCAgugagug,
(SEQ ID NO: 3599)
AGGguagaug,
(SEQ ID NO: 2888)
UGGguguguc,
(SEQ ID NO: 3600)
AUCgcgugag,
(SEQ ID NO: 3601)
AGGguaagcc,
(SEQ ID NO: 3602)
AGGguagcag,
(SEQ ID NO: 3603)
UUCguuuccg,
(SEQ ID NO: 147)
AAGguaagcg,
(SEQ ID NO: 2837)
UGGguaagcc,
(SEQ ID NO: 3604)
CAGguauggc,
(SEQ ID NO: 2907)
UGUguaagua,
(SEQ ID NO: 3605)
AAGguagaga,
(SEQ ID NO: 3606)
ACGguaauaa,
(SEQ ID NO: 3607)
CUGguacggu,
(SEQ ID NO: 3608)
GAGgucacag,
(SEQ ID NO: 2656)
UAUguaaguu,
(SEQ ID NO: 3609)
CUGguacgcc,
(SEQ ID NO: 1024)
CAAguaagau,
(SEQ ID NO: 1673)
CUAgugagua,
(SEQ ID NO: 3610)
CCGguaaccg,
(SEQ ID NO: 3611)
CUUguaaguc,
(SEQ ID NO: 2378)
GUGgugagaa,
(SEQ ID NO: 3612)
ACCguaugua,
(SEQ ID NO: 2324)
GUAguaagug,
(SEQ ID NO: 3014)
UUGgugggua,
(SEQ ID NO: 3613)
CGGguacuuu,
(SEQ ID NO: 2825)
UGGguaaaua,
(SEQ ID NO: 704)
AGAgugagua,
(SEQ ID NO: 230)
AAGguagguu,
(SEQ ID NO: 3614)
AAGguaugcg,
(SEQ ID NO: 3615)
CCUguaggcu,
(SEQ ID NO: 3616)
ACAguagaaa,
(SEQ ID NO: 3617)
CCGguuagua,
(SEQ ID NO: 3618)
CGGguaggcg,
(SEQ ID NO: 2162)
GCAgugagug,
(SEQ ID NO: 3619)
GAGgugaguc,
(SEQ ID NO: 3620)
CUGguagccu,
(SEQ ID NO: 1533)
CAUguaugua,
(SEQ ID NO: 3621)
GAAguaacuu,
(SEQ ID NO: 3622)
GAAguaagau,
(SEQ ID NO: 417)
AAGguuagau,
(SEQ ID NO: 161)
AAGguaauca,
(SEQ ID NO: 507)
AAUguaugua,
(SEQ ID NO: 2767)
UGAguaagau,
(SEQ ID NO: 703)
AGAgugagca,
(SEQ ID NO: 3623)
GUAguucuau,
(SEQ ID NO: 1898)
GAGguaauca,
(SEQ ID NO: 2548)
UAGguaugga,
(SEQ ID NO: 2612)
UAGgugggac,
(SEQ ID NO: 3624)
GAGguacaug,
(SEQ ID NO: 3625)
UGGguaaggc,
(SEQ ID NO: 1182)
CAGguacgcc,
(SEQ ID NO: 3626)
CCAguuacgc,
(SEQ ID NO: 3627)
ACUgugguga,
(SEQ ID NO: 1894)
GAGguaaguc,
(SEQ ID NO: 3628)
AUUguaggug,
(SEQ ID NO: 3629)
ACCgucagug,
(SEQ ID NO: 3630)
AAUgugaggg,
(SEQ ID NO: 645)
ACUgugagug,
(SEQ ID NO: 3631)
UGGguguggu,
(SEQ ID NO: 445)
AAGguuggga,
(SEQ ID NO: 464)
AAGguuugga,
(SEQ ID NO: 3632)
UCCgugagug,
(SEQ ID NO: 1642)
CGGgugagug,
(SEQ ID NO: 664)
AGAguaagcu,
(SEQ ID NO: 3633)
CAGgcaagcu,
(SEQ ID NO: 2541)
UAGguauauu,
(SEQ ID NO: 3634)
AAAguagcag,
(SEQ ID NO: 1880)
GAGguaaccu,
(SEQ ID NO: 379)
AAGgugggca,
(SEQ ID NO: 795)
AGGgugagua,
(SEQ ID NO: 2840)
UGGguaaggu,
(SEQ ID NO: 3635)
CUUgucagug,
(SEQ ID NO: 3636)
UAGgugcgcu,
(SEQ ID NO: 3637)
GAGgcaaauu,
(SEQ ID NO: 3638)
AGGguaccuc,
(SEQ ID NO: 3639)
CAAgugcgua,
(SEQ ID NO: 660)
AGAguaagac,
(SEQ ID NO: 3640)
GUGguaaaua,
(SEQ ID NO: 3641)
GAUguaagcg,
(SEQ ID NO: 1871)
GAGguaaagc,
(SEQ ID NO: 2596)
UAGgugagua,
(SEQ ID NO: 1130)
CAGguaacau,
(SEQ ID NO: 3642)
CCUguacggc,
(SEQ ID NO: 2552)
UAGguauguc,
(SEQ ID NO: 3643)
UAGguccaua,
(SEQ ID NO: 2003)
GAGgugaaaa,
(SEQ ID NO: 3644)
AAAguacuga,
(SEQ ID NO: 3645)
UUGguaagcg,
(SEQ ID NO: 3646)
CAGgcaagcg,
(SEQ ID NO: 3647)
UUUgcagguu,
(SEQ ID NO: 3648)
CAGguuuaua,
(SEQ ID NO: 1686)
CUGguaaagc,
(SEQ ID NO: 958)
AUGgugagcu,
(SEQ ID NO: 1419)
CAGgugguug,
(SEQ ID NO: 3649)
GUAguaaguu,
(SEQ ID NO: 3650)
CAGguaauac,
(SEQ ID NO: 3651)
CAGgcaaggc,
(SEQ ID NO: 171)
AAGguaauuu,
(SEQ ID NO: 3652)
UUUguccgug,
(SEQ ID NO: 1939)
GAGguagguu,
(SEQ ID NO: 3653)
ACCgugagug,
(SEQ ID NO: 3654)
CAAguaagcu,
(SEQ ID NO: 560)
ACAgugagua,
(SEQ ID NO: 3000)
UUGgugagau,
(SEQ ID NO: 233)
AAGguagucu,
(SEQ ID NO: 3655)
CAGguaaagg,
(SEQ ID NO: 2264)
GGGguaugga,
(SEQ ID NO: 3040)
UUUguaagug,
(SEQ ID NO: 2344)
GUGguaagag,
(SEQ ID NO: 838)
AGUgugaguu,
(SEQ ID NO: 3656)
AAGgcaagcg,
(SEQ ID NO: 2438)
UAAgugagua,
(SEQ ID NO: 797)
AGGgugagug,
(SEQ ID NO: 3657)
AGUguacgug,
(SEQ ID NO: 3658)
AGGgugcgua,
(SEQ ID NO: 2238)
GGCgugagcc,
(SEQ ID NO: 3659)
CGAguuauga,
(SEQ ID NO: 1122)
CAGguaaaga,
(SEQ ID NO: 3660)
UUGgugaaga,
(SEQ ID NO: 3661)
AGGguaaugg,
(SEQ ID NO: 300)
AAGguccaga,
(SEQ ID NO: 836)
AGUgugaguc,
(SEQ ID NO: 1159)
CAGguaauuu,
(SEQ ID NO: 3662)
CAGguaacgc,
(SEQ ID NO: 3663)
CUGguacacu,
(SEQ ID NO: 1782)
CUGguuagug,
(SEQ ID NO: 3664)
CAGguacuug,
(SEQ ID NO: 3665)
CACguaagua,
(SEQ ID NO: 3666)
GUGgugcggc,
(SEQ ID NO: 3667)
GAGgucaguu,
(SEQ ID NO: 932)
AUGguaugcc,
(SEQ ID NO: 405)
AAGgugugug,
(SEQ ID NO: 1772)
CUGguggguc,
(SEQ ID NO: 1342)
CAGgugaggc,
(SEQ ID NO: 423)
AAGguuaguc,
(SEQ ID NO: 215)
AAGguagcug,
(SEQ ID NO: 1983)
GAGgucagga,
(SEQ ID NO: 3668)
GUUguaggua,
(SEQ ID NO: 3669)
UGGguacaag,
(SEQ ID NO: 924)
AUGguaggug,
(SEQ ID NO: 1886)
GAGguaagcc,
(SEQ ID NO: 3670)
AUGgcaagua,
(SEQ ID NO: 245)
AAGguauauu,
(SEQ ID NO: 3671)
GCGgugagag,
(SEQ ID NO: 3672)
AAGgugcuuc,
(SEQ ID NO: 3673)
UAGguacauc,
(SEQ ID NO: 3674)
ACUgugguaa,
(SEQ ID NO: 1933)
GAGguaggcu,
(SEQ ID NO: 3675)
GAGguaugca,
(SEQ ID NO: 3676)
AGGguaguuc,
(SEQ ID NO: 1241)
CAGguauccu,
(SEQ ID NO: 741)
AGGguaaguc,
(SEQ ID NO: 779)
AGGgucaguu,
(SEQ ID NO: 3677)
CAGguuggga,
(SEQ ID NO: 3678)
CAGguggaua,
(SEQ ID NO: 2220)
GGAguagguu,
(SEQ ID NO: 3679)
GAGguaggau,
(SEQ ID NO: 3680)
GGGguuugug,
(SEQ ID NO: 3681)
UAGguaauug,
(SEQ ID NO: 136)
AAGguaaccc,
(SEQ ID NO: 3682)
ACGguaagaa,
(SEQ ID NO: 1936)
GAGguagggg,
(SEQ ID NO: 1619)
CGAguaggug,
(SEQ ID NO: 2710)
UCCguaagug,
(SEQ ID NO: 3683)
UCGguacagg,
(SEQ ID NO: 3684)
CAAguaagcg,
(SEQ ID NO: 3685)
AAGguccgcg,
(SEQ ID NO: 523)
AAUgugagua,
(SEQ ID NO: 3686)
CAGgugaaug,
(SEQ ID NO: 2350)
GUGguaaggc,
(SEQ ID NO: 706)
AGAgugagug,
(SEQ ID NO: 3687)
UCUguauguc,
(SEQ ID NO: 2876)
UGGgugaguc,
(SEQ ID NO: 3688)
UCGguuagua,
(SEQ ID NO: 3689)
GAUguaugca,
(SEQ ID NO: 3690)
GAGguuggug,
(SEQ ID NO: 2061)
GAGguggggc,
(SEQ ID NO: 3691)
UGGgucaguc,
(SEQ ID NO: 2161)
GCAgugagua,
(SEQ ID NO: 3692)
CAGguugcuu,
(SEQ ID NO: 3693)
AGGguagagu,
(SEQ ID NO: 2567)
UAGgucaggu,
(SEQ ID NO: 3694)
CGCguaugua,
(SEQ ID NO: 3695)
GAGguauuaa,
(SEQ ID NO: 3696)
CAGguaaacu,
(SEQ ID NO: 24)
AAAguaaguu,
(SEQ ID NO: 3697)
GGGgucuggc,
(SEQ ID NO: 3698)
GCUguggggu,
(SEQ ID NO: 3699)
UUGguaaguc,
(SEQ ID NO: 3700)
AAGguagaag,
(SEQ ID NO: 524)
AAUgugaguc,
(SEQ ID NO: 288)
AAGgucagcu,
(SEQ ID NO: 143)
AAGguaagag,
(SEQ ID NO: 3701)
AUGgugagga,
(SEQ ID NO: 200)
AAGguacuuc,
(SEQ ID NO: 141)
AAGguaagaa,
(SEQ ID NO: 3702)
CCGguacagc,
(SEQ ID NO: 3703)
GCGgugcgga,
(SEQ ID NO: 1168)
CAGguacaua,
(SEQ ID NO: 1755)
CUGgugagga,
(SEQ ID NO: 1731)
CUGguaggug,
(SEQ ID NO: 3704)
AACguagguu,
(SEQ ID NO: 975)
AUGgugugug,
(SEQ ID NO: 3705)
UUGguacuau,
(SEQ ID NO: 1300)
CAGgucggug,
(SEQ ID NO: 3706)
CAGgcauggg,
(SEQ ID NO: 929)
AUGguaucuu,
(SEQ ID NO: 137)
AAGguaacua,
(SEQ ID NO: 3707)
CAGgugggcg,
(SEQ ID NO: 3708)
CACgugagga,
(SEQ ID NO: 392)
AAGgugguuc,
(SEQ ID NO: 3709)
UGGgcauucu,
(SEQ ID NO: 894)
AUGguaagcc,
(SEQ ID NO: 778)
AGGgucagug,
(SEQ ID NO: 3710)
AGAguacgua,
(SEQ ID NO: 220)
AAGguaggca,
(SEQ ID NO: 3711)
AAGguauuca,
(SEQ ID NO: 1202)
CAGguagauu,
(SEQ ID NO: 1972)
GAGguauuua,
(SEQ ID NO: 3712)
GAGgucuaca,
(SEQ ID NO: 3713)
GUUguagguc,
(SEQ ID NO: 3714)
CAGguacucg,
(SEQ ID NO: 3715)
GUCguauguu,
(SEQ ID NO: 202)
AAGguacuuu,
(SEQ ID NO: 702)
AGAgugagau,
(SEQ ID NO: 3716)
AGUguuggua,
(SEQ ID NO: 525)
AAUgugagug,
(SEQ ID NO: 3717)
AAGguagauu,
(SEQ ID NO: 988)
AUGguuugua,
(SEQ ID NO: 3718)
GAGgccccag,
(SEQ ID NO: 3719)
AUGgucaguu,
(SEQ ID NO: 3720)
UCUguaagga,
(SEQ ID NO: 3721)
CAGgucgggc,
(SEQ ID NO: 1142)
CAGguaagcc,
(SEQ ID NO: 2569)
UAGgucagug,
(SEQ ID NO: 683)
AGAguaggaa,
(SEQ ID NO: 3722)
CUGguacuuc,
(SEQ ID NO: 1674)
CUCguaagca,
(SEQ ID NO: 1134)
CAGguaacua,
(SEQ ID NO: 1401)
CAGguggcug,
(SEQ ID NO: 3723)
UGGguccgua,
(SEQ ID NO: 3724)
GAGguugugc,
(SEQ ID NO: 1377)
CAGgugcgcg,
(SEQ ID NO: 3725)
AAAguauggc,
(SEQ ID NO: 2779)
UGAguacgua,
(SEQ ID NO: 3726)
CUGguacgga,
(SEQ ID NO: 3727)
CAAgugaccu,
(SEQ ID NO: 356)
AAGgugaugu,
(SEQ ID NO: 3728)
AAGgucugca,
(SEQ ID NO: 75)
AAAguuugua,
(SEQ ID NO: 339)
AAGgugagca,
(SEQ ID NO: 2119)
GAUguaagcc,
(SEQ ID NO: 1035)
CAAguaauuu,
(SEQ ID NO: 1442)
CAGgugugug,
(SEQ ID NO: 2874)
UGGgugaggg,
(SEQ ID NO: 3729)
AAGgugaccu,
(SEQ ID NO: 2621)
UAGgugugag,
(SEQ ID NO: 3730)
CAGgcagguc,
(SEQ ID NO: 2692)
UCAguaaguu,
(SEQ ID NO: 3731)
UCAgcaguga,
(SEQ ID NO: 3732)
AAGguaccac,
(SEQ ID NO: 3733)
UAAguaggug,
(SEQ ID NO: 286)
AAGgucagcc,
(SEQ ID NO: 1135)
CAGguaacuc,
(SEQ ID NO: 13)
AAAguaagag,
(SEQ ID NO: 209)
AAGguagaua,
(SEQ ID NO: 99)
AAGgcaaggg,
(SEQ ID NO: 3734)
CAGgugucgg,
(SEQ ID NO: 3735)
CAGguggcua,
(SEQ ID NO: 3736)
GAGguugcca,
(SEQ ID NO: 3737)
CAGgccgugg,
(SEQ ID NO: 3738)
UUGguauaug,
(SEQ ID NO: 3739)
GAGguugagu,
(SEQ ID NO: 3740)
GAGguagguc,
(SEQ ID NO: 2343)
GUGguaagac,
(SEQ ID NO: 3741)
UAGguccuuc,
(SEQ ID NO: 3742)
GAGgcaaguc,
(SEQ ID NO: 3743)
GAGguaacau,
(SEQ ID NO: 1236)
CAGguauauc,
(SEQ ID NO: 3744)
UCGguugguu,
(SEQ ID NO: 3745)
CAGgugaacc,
(SEQ ID NO: 3746)
CAGgucuuuu,
(SEQ ID NO: 3747)
CAGgcauggc,
(SEQ ID NO: 32)
AAAguacuug,
(SEQ ID NO: 1356)
CAGgugauuc,
(SEQ ID NO: 3748)
UUGguagguu,
(SEQ ID NO: 3749)
UAUgugagca,
(SEQ ID NO: 1339)
CAGgugagcg,
(SEQ ID NO: 3750)
AAUguaauaa,
(SEQ ID NO: 3751)
AAAguaaggc,
(SEQ ID NO: 2644)
UAGguuuguc,
(SEQ ID NO: 2613)
UAGgugggag,
(SEQ ID NO: 3752)
GAGguaaguu,
(SEQ ID NO: 3753)
AAGguagccg,
(SEQ ID NO: 3754)
CAGguggugc,
(SEQ ID NO: 3755)
UGAgucaguu,
(SEQ ID NO: 3756)
CUGguaggcc,
(SEQ ID NO: 3757)
CAAguaagga,
(SEQ ID NO: 3758)
CGGguaaggc,
(SEQ ID NO: 3759)
AAGgcgagga,
(SEQ ID NO: 1230)
CAGguaguuc,
(SEQ ID NO: 1143)
CAGguaagga,
(SEQ ID NO: 1610)
CCUgugagug,
(SEQ ID NO: 132)
AAGguaaaug,
(SEQ ID NO: 3760)
CCGguaauua,
(SEQ ID NO: 1149)
CAGguaaguu,
(SEQ ID NO: 3761)
AAGgugguca,
(SEQ ID NO: 1177)
CAGguaccuc,
(SEQ ID NO: 3762)
AUCguaagua,
(SEQ ID NO: 3763)
CCGguacaua,
(SEQ ID NO: 3764)
GCGgugagug,
(SEQ ID NO: 2067)
GAGgugguau,
(SEQ ID NO: 3765)
CUGgugugga,
(SEQ ID NO: 3766)
GAGguaauuc,
(SEQ ID NO: 3767)
CAAguacgua,
(SEQ ID NO: 2746)
UCUguaagug,
(SEQ ID NO: 491)
AAUguaagug,
(SEQ ID NO: 783)
AGGgucuguu,
(SEQ ID NO: 1918)
GAGguacugc,
(SEQ ID NO: 738)
AGGguaaggc,
(SEQ ID NO: 95)
AAGgcaagag,
(SEQ ID NO: 1416)
CAGguggguu,
(SEQ ID NO: 3768)
UAGguuagga,
(SEQ ID NO: 2769)
UGAguaagcu,
(SEQ ID NO: 661)
AGAguaagag,
(SEQ ID NO: 3769)
AUGgcaggug,
(SEQ ID NO: 3770)
UAGgcaagua,
(SEQ ID NO: 923)
AUGguaggua,
(SEQ ID NO: 3771)
GCAgcccgca,
(SEQ ID NO: 3772)
ACGguaaacu,
(SEQ ID NO: 798)
AGGgugaguu,
(SEQ ID NO: 3773)
GUAguagucu,
(SEQ ID NO: 3774)
GUGgcugaaa,
(SEQ ID NO: 1456)
CAGguuaguc,
(SEQ ID NO: 1753)
CUGgugagca,
(SEQ ID NO: 2691)
UCAguaagug,
(SEQ ID NO: 3775)
AAAgugauug,
(SEQ ID NO: 3776)
UAGgucugga,
(SEQ ID NO: 3777)
GAGguguuuc,
(SEQ ID NO: 133)
AAGguaaauu,
(SEQ ID NO: 3778)
CAUguacauc,
(SEQ ID NO: 3779)
AAGguuugaa,
(SEQ ID NO: 3780)
CCAgcaagug,
(SEQ ID NO: 3781)
UAGguaauaa,
(SEQ ID NO: 1859)
GAGgcaagug,
(SEQ ID NO: 1071)
CAAgugauuc,
(SEQ ID NO: 3782)
CAGgucgugg,
(SEQ ID NO: 3783)
GAAguaugcc,
(SEQ ID NO: 3784)
UCGgugcccu,
(SEQ ID NO: 3785)
GAGgucaguc,
(SEQ ID NO: 1334)
CAGgugagac,
(SEQ ID NO: 3786)
UUUgucugua,
(SEQ ID NO: 3787)
CAGguagaua
(SEQ ID NO: 3788)
UGGguaucag,
(SEQ ID NO: 2616)
UAGgugggcu,
(SEQ ID NO: 3789)
AUGgugagau,
(SEQ ID NO: 3790)
CAGguaacac,
(SEQ ID NO: 3791)
CCGguauccu,
(SEQ ID NO: 2487)
UAGguaagcu,
(SEQ ID NO: 3792)
UCAguacauc,
(SEQ ID NO: 2642)
UAGguuugcc,
(SEQ ID NO: 889)
AUGguaagaa,
(SEQ ID NO: 3793)
UUGguaagac,
(SEQ ID NO: 3794)
CCGguuaguc,
(SEQ ID NO: 1882)
GAGguaagaa,
(SEQ ID NO: 2844)
UGGguaaguu,
(SEQ ID NO: 1585)
CCGgugagaa,
(SEQ ID NO: 1608)
CCUgugaggg,
(SEQ ID NO: 590)
ACGguaggag,
(SEQ ID NO: 3795)
ACAguauguc,
(SEQ ID NO: 3796)
CAGguauuaa,
(SEQ ID NO: 3797)
CAGguggauc,
(SEQ ID NO: 3798)
AGAgugcgua,
(SEQ ID NO: 3799)
AAGgugaccg,
(SEQ ID NO: 687)
AGAguaggug,
(SEQ ID NO: 3800)
ACUguaugua,
(SEQ ID NO: 3801)
UAGgucaauu,
(SEQ ID NO: 3802)
AGUguguaag,
(SEQ ID NO: 3803)
CGGguaccuu,
(SEQ ID NO: 3804)
CUAgugaguu,
(SEQ ID NO: 1666)
CUAguaagug,
(SEQ ID NO: 3805)
CAGguacaac,
(SEQ ID NO: 2627)
UAGgugugug,
(SEQ ID NO: 3806)
CAUguacggc,
(SEQ ID NO: 3807)
AUGgugugag,
(SEQ ID NO: 3808)
AGGguggaag,
(SEQ ID NO: 3809)
CAGgugcgag,
(SEQ ID NO: 3810)
UAGgugcucc,
(SEQ ID NO: 390)
AAGguggugg,
(SEQ ID NO: 317)
AAGgucuguu,
(SEQ ID NO: 1407)
CAGgugggcc,
(SEQ ID NO: 294)
AAGgucaguc,
(SEQ ID NO: 3811)
CAGguuuuua,
(SEQ ID NO: 3812)
AACgugaggu,
(SEQ ID NO: 3813)
CGGguaagag,
(SEQ ID NO: 3814)
UUUgucggua,
(SEQ ID NO: 3815)
UAGguuaagu,
(SEQ ID NO: 2342)
GUGguaagaa,
(SEQ ID NO: 1266)
CAGguauugg,
(SEQ ID NO: 2196)
GCUguaaguu,
(SEQ ID NO: 1664)
CUAguaagua,
(SEQ ID NO: 3816)
UCGguaaaua,
(SEQ ID NO: 1137)
CAGguaacuu,
(SEQ ID NO: 3817)
CCUgugagua,
(SEQ ID NO: 3818)
CAGguuauau,
(SEQ ID NO: 3819)
CUGgugaaca,
(SEQ ID NO: 238)
AAGguauaaa,
(SEQ ID NO: 1885)
GAGguaagca,
(SEQ ID NO: 324)
AAGgugaagc,
(SEQ ID NO: 1348)
CAGgugaguu,
(SEQ ID NO: 3820)
UUUgugagua,
(SEQ ID NO: 3821)
CUUguacgcc,
(SEQ ID NO: 670)
AGAguaagug,
(SEQ ID NO: 2853)
UGGguaggug,
(SEQ ID NO: 3822)
UGAgcccugc,
(SEQ ID NO: 3823)
UGUguaugua,
(SEQ ID NO: 3824)
AAGguagagg,
(SEQ ID NO: 2062)
GAGguggggg,
(SEQ ID NO: 2502)
UAGguaauuc,
(SEQ ID NO: 3825)
AAGgcauggu,
(SEQ ID NO: 663)
AGAguaagca,
(SEQ ID NO: 217)
AAGguaggaa,
(SEQ ID NO: 1026)
CAAguaagua,
(SEQ ID NO: 3826)
ACUguaauug,
(SEQ ID NO: 1311)
CAGgucugug,
(SEQ ID NO: 3827)
UCGguaccga,
(SEQ ID NO: 3828)
CUGgugagag,
(SEQ ID NO: 463)
AAGguuugcu,
(SEQ ID NO: 3829)
AUGguaccac,
(SEQ ID NO: 3830)
UAAguuaguu,
(SEQ ID NO: 1213)
CAGguaggac,
(SEQ ID NO: 3831)
AGAgugaggc,
(SEQ ID NO: 3832)
CGAgucagua,
(SEQ ID NO: 1304)
CAGgucugag,
(SEQ ID NO: 3833)
GAGguggugg,
(SEQ ID NO: 3834)
ACGguauugg,
(SEQ ID NO: 3835)
GCUgcgagua,
(SEQ ID NO: 1708)
CUGguaagug,
(SEQ ID NO: 2379)
GUGgugagau,
(SEQ ID NO: 3836)
GGGguuugau,
(SEQ ID NO: 2762)
UCUgugagug,
(SEQ ID NO: 1801)
CUUgucagua,
(SEQ ID NO: 1866)
GAGguaaaac,
(SEQ ID NO: 2741)
UCUguaagau,
(SEQ ID NO: 1558)
CCAguaaguu,
(SEQ ID NO: 1124)
CAGguaaagu,
(SEQ ID NO: 2179)
GCGgugagca,
(SEQ ID NO: 2416)
UAAguaagag,
(SEQ ID NO: 3837)
CUGgcaggug,
(SEQ ID NO: 1891)
GAGguaaggg,
(SEQ ID NO: 2775)
UGAguaaguu,
(SEQ ID NO: 2015)
GAGgugagac,
(SEQ ID NO: 3838)
GCUgucuguu,
(SEQ ID NO: 134)
AAGguaacaa,
(SEQ ID NO: 3839)
GAGguaacgg,
(SEQ ID NO: 3840)
CUGguauucu,
(SEQ ID NO: 1021)
CAAguaacug,
(SEQ ID NO: 383)
AAGguggggu,
(SEQ ID NO: 2549)
UAGguauggc,
(SEQ ID NO: 1271)
CAGguauuuu,
(SEQ ID NO: 3841)
GUGguaaacu,
(SEQ ID NO: 1998)
GAGgucugag,
(SEQ ID NO: 1706)
CUGguaaggu,
(SEQ ID NO: 1029)
CAAguaaguu,
(SEQ ID NO: 206)
AAGguagacc,
(SEQ ID NO: 3842)
GAGgcgagcg,
(SEQ ID NO: 1687)
CUGguaaaua,
(SEQ ID NO: 3843)
UGUguaagcg,
(SEQ ID NO: 1453)
CAGguuaggg,
(SEQ ID NO: 2280)
GGGgugagga,
(SEQ ID NO: 3844)
ACAguaugug,
(SEQ ID NO: 3845)
CCGgugggga,
(SEQ ID NO: 3846)
GAGgucagug,
(SEQ ID NO: 3847)
AGGguaaggu,
(SEQ ID NO: 546)
ACAguaagua,
(SEQ ID NO: 3848)
GGUguaaggu,
(SEQ ID NO: 1895)
GAGguaauaa,
(SEQ ID NO: 3849)
CAGguauucc,
(SEQ ID NO: 3850)
CUGguauaaa,
(SEQ ID NO: 3851)
CCGgucugug,
(SEQ ID NO: 1136)
CAGguaacug,
(SEQ ID NO: 2147)
GCAguaagua,
(SEQ ID NO: 225)
AAGguagggg,
(SEQ ID NO: 3852)
CAAguccacc,
(SEQ ID NO: 3853)
CAAguuggug,
(SEQ ID NO: 1379)
CAGgugcggu,
(SEQ ID NO: 3854)
CAGguaaaau,
(SEQ ID NO: 3855)
ACGguaagga,
(SEQ ID NO: 3856)
UGGguaauaa,
(SEQ ID NO: 2493)
UAGguaagug,
(SEQ ID NO: 3857)
CCGguagguu,
(SEQ ID NO: 3858)
AGAguaugga,
(SEQ ID NO: 3859)
CUCgugaguc,
(SEQ ID NO: 3860)
AAAgccggug,
(SEQ ID NO: 2970)
UUGguaauuu,
(SEQ ID NO: 1867)
GAGguaaaag,
(SEQ ID NO: 3861)
CCUgugugag,
(SEQ ID NO: 18)
AAAguaagga,
(SEQ ID NO: 2800)
UGAgugagug,
(SEQ ID NO: 180)
AAGguacaug,
(SEQ ID NO: 3862)
CCGguaaaug,
(SEQ ID NO: 3863)
CAGgugaagc,
(SEQ ID NO: 1173)
CAGguacccg,
(SEQ ID NO: 1890)
GAGguaaggc,
(SEQ ID NO: 3049)
UUUguauguu,
(SEQ ID NO: 1386)
CAGgugcucc,
(SEQ ID NO: 3864)
UCGguagguc,
(SEQ ID NO: 3865)
CGGgugaggc,
(SEQ ID NO: 168)
AAGguaauua,
(SEQ ID NO: 644)
ACUgugaguc,
(SEQ ID NO: 285)
AAGgucagca,
(SEQ ID NO: 2384)
GUGgugagug,
(SEQ ID NO: 3866)
CAUguccacc,
(SEQ ID NO: 3867)
AAGgugaccc,
(SEQ ID NO: 3868)
CGGguuagua,
(SEQ ID NO: 3869)
GCGguaguaa,
(SEQ ID NO: 3870)
GCUguaggua,
(SEQ ID NO: 3871)
CCUguugagu,
(SEQ ID NO: 2577)
UAGgucuggc,
(SEQ ID NO: 2131)
GAUgugagcc,
(SEQ ID NO: 1802)
CUUgugagua,
(SEQ ID NO: 1780)
CUGguguguu,
(SEQ ID NO: 1863)
GAGgcaugug,
(SEQ ID NO: 1101)
CAGgcaagag,
(SEQ ID NO: 2957)
UUGguaagaa,
(SEQ ID NO: 2075)
GAGguguggg,
(SEQ ID NO: 1975)
GAGguauuuu,
(SEQ ID NO: 1224)
CAGguaguaa,
(SEQ ID NO: 3872)
AGGguaagac,
(SEQ ID NO: 3873)
UUUguaggca,
(SEQ ID NO: 3874)
AGGgugagau,
(SEQ ID NO: 2110)
GAGguuugua,
(SEQ ID NO: 349)
AAGgugagug,
(SEQ ID NO: 2055)
GAGgugggag,
(SEQ ID NO: 335)
AAGgugagaa,
(SEQ ID NO: 1698)
CUGguaagag,
(SEQ ID NO: 3875)
AUAguaaaga,
(SEQ ID NO: 2134)
GAUgugaguc,
(SEQ ID NO: 3876)
AAGgugcagg,
(SEQ ID NO: 1310)
CAGgucuguc,
(SEQ ID NO: 3877)
GAGgugauuu,
(SEQ ID NO: 3878)
CAGguuggcu,
(SEQ ID NO: 3879)
CGGguauggg,
(SEQ ID NO: 3880)
AUGguccauc,
(SEQ ID NO: 3881)
CCGguuggug,
(SEQ ID NO: 3882)
GGAguaaguc,
(SEQ ID NO: 488)
AAUguaagga,
(SEQ ID NO: 1510)
CAGguuuguu,
(SEQ ID NO: 2626)
UAGgugugua,
(SEQ ID NO: 3883)
UAUgucuuug,
(SEQ ID NO: 3884)
ACGguacuuc,
(SEQ ID NO: 3885)
AAGgcacgcg,
(SEQ ID NO: 1684)
CUGguaaacc,
(SEQ ID NO: 3886)
CUUgugggua,
(SEQ ID NO: 2773)
UGAguaaguc,
(SEQ ID NO: 1773)
CUGgugggug,
(SEQ ID NO: 3887)
GAGguggaga,
(SEQ ID NO: 3888)
GUGguggcug,
(SEQ ID NO: 2353)
GUGguaagug,
(SEQ ID NO: 3889)
AACgugagua,
(SEQ ID NO: 3890)
GAAgcuguaa,
(SEQ ID NO: 3891)
CGGguaucuu,
(SEQ ID NO: 1424)
CAGgugucag,
(SEQ ID NO: 3892)
AAUguacgca,
(SEQ ID NO: 3893)
CCGgugggua,
(SEQ ID NO: 3894)
UGGgugaggu,
(SEQ ID NO: 266)
AAGguauguu,
(SEQ ID NO: 1261)
CAGguauguu,
(SEQ ID NO: 1505)
CAGguuugcu,
(SEQ ID NO: 2964)
UUGguaaguu,
(SEQ ID NO: 1231)
CAGguaguug,
(SEQ ID NO: 3895)
CCUgugaaua,
(SEQ ID NO: 3896)
GCUgugugug,
(SEQ ID NO: 1033)
CAAguaauuc,
(SEQ ID NO: 3897)
AGGguaaugu,
(SEQ ID NO: 2205)
GCUgugaguc,
(SEQ ID NO: 3898)
ACCguaaguu,
(SEQ ID NO: 3899)
CGUguaagua,
(SEQ ID NO: 3900)
GGGguaaguc,
(SEQ ID NO: 3901)
AAUguaugau,
(SEQ ID NO: 3902)
AAUgugauua,
(SEQ ID NO: 2682)
UCAguaagaa,
(SEQ ID NO: 3903)
CAGguccguc,
(SEQ ID NO: 3904)
GAAguauuga,
(SEQ ID NO: 2960)
UUGguaagga,
(SEQ ID NO: 3905)
CAGgucgguu,
(SEQ ID NO: 2635)
UAGguuagug,
(SEQ ID NO: 577)
ACGguaaaac,
(SEQ ID NO: 228)
AAGguagguc,
(SEQ ID NO: 3906)
UACgugagua,
(SEQ ID NO: 3907)
UUGguaagca,
(SEQ ID NO: 3908)
GCGgugaguc,
(SEQ ID NO: 3909)
GAAguaaggg,
(SEQ ID NO: 3910)
CGCgugaguu,
(SEQ ID NO: 3911)
CAGguacccc,
(SEQ ID NO: 3912)
UCUguaagac,
(SEQ ID NO: 2057)
GAGgugggca,
(SEQ ID NO: 3913)
AAUguaagac,
(SEQ ID NO: 3914)
CAGgcaaggg,
(SEQ ID NO: 1020)
CAAguaacua,
(SEQ ID NO: 3915)
AAAguuuguc,
(SEQ ID NO: 1193)
CAGguacugu,
(SEQ ID NO: 303)
AAGgucccuc,
(SEQ ID NO: 3916)
UCGguaaguc,
(SEQ ID NO: 2877)
UGGgugagug,
(SEQ ID NO: 3917)
CUUgugagau,
(SEQ ID NO: 3918)
AGAgugagcu,
(SEQ ID NO: 3919)
UAAgugggga,
(SEQ ID NO: 2522)
UAGguaggga,
(SEQ ID NO: 1452)
CAGguuagcc,
(SEQ ID NO: 3920)
AGGguaauca,
(SEQ ID NO: 3921)
AAGguucagc,
(SEQ ID NO: 2885)
UGGgugggug,
(SEQ ID NO: 1494)
CAGguuguga,
(SEQ ID NO: 155)
AAGguaagug,
(SEQ ID NO: 1543)
CAUgugcgua,
(SEQ ID NO: 3922)
CCGguauauu,
(SEQ ID NO: 3923)
ACCguaugug,
(SEQ ID NO: 3924)
CAGguauagu,
(SEQ ID NO: 3925)
CAGguauuac,
(SEQ ID NO: 1364)
CAGgugcagg,
(SEQ ID NO: 2381)
GUGgugagcu,
(SEQ ID NO: 135)
AAGguaacau,
(SEQ ID NO: 3926)
CUGgugaugg,
(SEQ ID NO: 882)
AUGguaaaug,
(SEQ ID NO: 3927)
CCGgugagca,
(SEQ ID NO: 124)
AAGguaaacc,
(SEQ ID NO: 3928)
AAGguacugg,
(SEQ ID NO: 3929)
GCGgucagga,
(SEQ ID NO: 3930)
CUGgucaggg,
(SEQ ID NO: 3931)
AAAguacguu,
(SEQ ID NO: 688)
AGAguagguu,
(SEQ ID NO: 3932)
AGGguaagcu,
(SEQ ID NO: 1009)
AUUgugagua,
(SEQ ID NO: 3933)
CCGgccacca,
(SEQ ID NO: 1881)
GAGguaacuu,
(SEQ ID NO: 1956)
GAGguaugaa,
(SEQ ID NO: 1276)
CAGgucagac,
(SEQ ID NO: 2462)
UAGgcgugug,
(SEQ ID NO: 743)
AGGguaaguu,
(SEQ ID NO: 1111)
CAGgcaugag,
(SEQ ID NO: 1133)
CAGguaacgu,
(SEQ ID NO: 3934)
CAGgcgagca,
(SEQ ID NO: 2550)
UAGguauggu,
(SEQ ID NO: 3935)
AGAguaggau,
(SEQ ID NO: 3936)
CUGguuucaa,
(SEQ ID NO: 3937)
GAGguaaacu,
(SEQ ID NO: 1112)
CAGgcaugca,
(SEQ ID NO: 3938)
UUGguaaucu,
(SEQ ID NO: 3939)
AGGgcagaau,
(SEQ ID NO: 877)
AUGguaaaac,
(SEQ ID NO: 3940)
GCUgcaggug,
(SEQ ID NO: 3941)
GAAgcacgug,
(SEQ ID NO: 3942)
CAUguaaaca,
(SEQ ID NO: 2835)
UGGguaagau,
(SEQ ID NO: 3943)
AGGguagcua,
(SEQ ID NO: 800)
AGGguggggu,
(SEQ ID NO: 1600)
CCUguaaguu,
(SEQ ID NO: 2801)
UGAgugaguu,
(SEQ ID NO: 3944)
GGAguaugua,
(SEQ ID NO: 1328)
CAGgugaccu,
(SEQ ID NO: 3945)
AAAguacgga,
(SEQ ID NO: 1906)
GAGguacaga,
(SEQ ID NO: 2125)
GAUguaggua,
(SEQ ID NO: 3946)
GGGguaauug,
(SEQ ID NO: 2617)
UAGguggguu,
(SEQ ID NO: 3947)
GUGguacgua,
(SEQ ID NO: 3948)
AAGguacagc,
(SEQ ID NO: 3949)
GAGgugaaga,
(SEQ ID NO: 2246)
GGGguaagca,
(SEQ ID NO: 3950)
UGAguagguc,
(SEQ ID NO: 2253)
GGGguaaguu,
(SEQ ID NO: 1011)
AUUgugaguu,
(SEQ ID NO: 3951)
UCAguaagac,
(SEQ ID NO: 834)
AGUgugagcu,
(SEQ ID NO: 3952)
AAGgcaaaac,
(SEQ ID NO: 1760)
CUGgugaguc,
(SEQ ID NO: 310)
AAGgucucug,
(SEQ ID NO: 3953)
GAGgcugugc,
(SEQ ID NO: 700)
AGAgugagac,
(SEQ ID NO: 2033)
GAGgugaugu,
(SEQ ID NO: 3954)
AGAguauggu,
(SEQ ID NO: 2884)
UGGguggguc,
(SEQ ID NO: 3955)
GCUgcugagc,
(SEQ ID NO: 1210)
CAGguagcug,
(SEQ ID NO: 3956)
UAGgucagaa,
(SEQ ID NO: 3957)
CCGguaggug,
(SEQ ID NO: 3958)
GCAguaugau,
(SEQ ID NO: 3959)
CAGguuucag,
(SEQ ID NO: 3960)
GAGguuugcc,
(SEQ ID NO: 3961)
GGGguggggg,
(SEQ ID NO: 179)
AAGguacaua,
(SEQ ID NO: 2890)
UGGguguguu,
(SEQ ID NO: 666)
AGAguaaggc,
(SEQ ID NO: 3962)
GCGguuagug,
(SEQ ID NO: 334)
AAGgugacuu,
(SEQ ID NO: 892)
AUGguaagau,
(SEQ ID NO: 3963)
AUGguaguug,
(SEQ ID NO: 3964)
CAUguaagac,
(SEQ ID NO: 1736)
CUGguaugua,
(SEQ ID NO: 3965)
UUCguaagga,
(SEQ ID NO: 3966)
GAAguaugac,
(SEQ ID NO: 1627)
CGGguaauuc,
(SEQ ID NO: 2831)
UGGguaacuu,
(SEQ ID NO: 1372)
CAGgugccua,
(SEQ ID NO: 3967)
CAUguagggc,
(SEQ ID NO: 3968)
ACCgucagga,
(SEQ ID NO: 3969)
CGUguucgau,
(SEQ ID NO: 3970)
GAGgcaggac,
(SEQ ID NO: 2496)
UAGguaauau,
(SEQ ID NO: 3971)
UCGguauacu,
(SEQ ID NO: 3972)
UAGguugugc,
(SEQ ID NO: 3973)
CCGgugaguc,
(SEQ ID NO: 1368)
CAGgugccaa,
(SEQ ID NO: 1352)
CAGgugaugc,
(SEQ ID NO: 343)
AAGgugagga,
(SEQ ID NO: 3974)
GUGgugaggg,
(SEQ ID NO: 3975)
UGGgucagua,
(SEQ ID NO: 1985)
GAGgucaggg,
(SEQ ID NO: 2511)
UAGguacgua,
(SEQ ID NO: 1857)
GAGgcaagag,
(SEQ ID NO: 3976)
CCUguuggua,
(SEQ ID NO: 3977)
GAGguaucca,
(SEQ ID NO: 2419)
UAAguaagcu,
(SEQ ID NO: 296)
AAGgucaguu,
(SEQ ID NO: 3978)
AAAguuaaag,
(SEQ ID NO: 3979)
GAGgugcuau,
(SEQ ID NO: 581)
ACGguaaguu,
(SEQ ID NO: 1757)
CUGgugaggg,
(SEQ ID NO: 2091)
GAGguuaugu,
(SEQ ID NO: 3980)
CUUgugugca,
(SEQ ID NO: 3981)
UGAgcugggg,
(SEQ ID NO: 3982)
AAGguauagu,
(SEQ ID NO: 2464)
UAGguaaaac,
(SEQ ID NO: 3983)
GGGgugaggu,
(SEQ ID NO: 3984)
GAGgcaagca,
(SEQ ID NO: 3985)
GGAguaacgu,
(SEQ ID NO: 3986)
AGAguaagua,
(SEQ ID NO: 21)
AAAguaagua,
(SEQ ID NO: 3987)
GAGgcaacca,
(SEQ ID NO: 2909)
UGUguaaguu,
(SEQ ID NO: 2594)
UAGgugaggc,
(SEQ ID NO: 544)
ACAguaagaa,
(SEQ ID NO: 2774)
UGAguaagug,
(SEQ ID NO: 1057)
CAAgucagua,
(SEQ ID NO: 3988)
AGGguaaaug,
(SEQ ID NO: 257)
AAGguaugca,
(SEQ ID NO: 3989)
GCUgugcgug,
(SEQ ID NO: 3990)
GAGguucgcc,
(SEQ ID NO: 3991)
AAGgcuugca,
(SEQ ID NO: 1104)
CAGgcaagug,
(SEQ ID NO: 3992)
AUAguaaguc,
(SEQ ID NO: 2978)
UUGguaggua,
(SEQ ID NO: 3993)
GCAgcaggua,
(SEQ ID NO: 243)
AAGguauauc,
(SEQ ID NO: 3994)
AGCguaagcc,
(SEQ ID NO: 3995)
CUGguucgaa,
(SEQ ID NO: 612)
ACGgugggug,
(SEQ ID NO: 3996)
CUGgucauug,
(SEQ ID NO: 1280)
CAGgucagga,
(SEQ ID NO: 1062)
CAAgugagac,
(SEQ ID NO: 1919)
GAGguacugg,
(SEQ ID NO: 3997)
GAGguguagu,
(SEQ ID NO: 3998)
GAGguguccu,
(SEQ ID NO: 1380)
CAGgugcgua,
(SEQ ID NO: 3999)
AGUgcccuga,
(SEQ ID NO: 962)
AUGgugaguc,
(SEQ ID NO: 4000)
UGUgugugua,
(SEQ ID NO: 1254)
CAGguaugcu,
(SEQ ID NO: 4001)
CUGguacagu,
(SEQ ID NO: 4002)
UUGguacgua,
(SEQ ID NO: 4003)
UCUguacgua,
(SEQ ID NO: 4004)
UAAguaauuc,
(SEQ ID NO: 4005)
CACguaugug,
(SEQ ID NO: 1103)
CAGgcaagua,
(SEQ ID NO: 4006)
UCGgugagug,
(SEQ ID NO: 2315)
GGUgugaguc,
(SEQ ID NO: 2743)
UCUguaagcu,
(SEQ ID NO: 4007)
AAGguucaga,
(SEQ ID NO: 4008)
AGGguacuuc,
(SEQ ID NO: 4009)
GCGgcagguu,
(SEQ ID NO: 4010)
GAGgcccgug,
(SEQ ID NO: 4011)
CAGguauaaa,
(SEQ ID NO: 4012)
AUGgucaagu,
(SEQ ID NO: 347)
AAGgugagua,
(SEQ ID NO: 4013)
GUGguuuguu,
(SEQ ID NO: 4014)
AGAgugagga,
(SEQ ID NO: 1957)
GAGguaugac,
(SEQ ID NO: 4015)
UAGgcgugag,
(SEQ ID NO: 4016)
AAGguacucc,
(SEQ ID NO: 2798)
UGAgugagga,
(SEQ ID NO: 4017)
GAGguaugau,
(SEQ ID NO: 4018)
GGGgucggua,
(SEQ ID NO: 4019)
ACGguaugca,
(SEQ ID NO: 1171)
CAGguaccac,
(SEQ ID NO: 4020)
UAAguaccug,
(SEQ ID NO: 4021)
AGGgugggcu,
(SEQ ID NO: 4022)
CUGgucuguu,
(SEQ ID NO: 4023)
UAGgucagag,
(SEQ ID NO: 406)
AAGguguguu,
(SEQ ID NO: 4024)
CUGgucagug,
(SEQ ID NO: 4025)
AAGgugggac,
(SEQ ID NO: 4026)
GUGguaguag,
(SEQ ID NO: 4027)
CUAguuuagg,
(SEQ ID NO: 4028)
CCCgccccau,
(SEQ ID NO: 4029)
GCUguacugc,
(SEQ ID NO: 1897)
GAGguaauau,
(SEQ ID NO: 4030)
UAGguuggug,
(SEQ ID NO: 4031)
AAGguccaac,
(SEQ ID NO: 2593)
UAGgugagga,
(SEQ ID NO: 2354)
GUGguaaguu,
(SEQ ID NO: 831)
AGUgugagag,
(SEQ ID NO: 497)
AAUguacaug,
(SEQ ID NO: 4032)
UUGgcaggug,
(SEQ ID NO: 4033)
UAGguuauug,
(SEQ ID NO: 1191)
CAGguacuga,
(SEQ ID NO: 4034)
GCGguggguc,
(SEQ ID NO: 4035)
UGUguaagau,
(SEQ ID NO: 2025)
GAGgugagua,
(SEQ ID NO: 4036)
GCAgccccgg,
(SEQ ID NO: 4037)
CAGgugcuaa,
(SEQ ID NO: 815)
AGUguaagag,
(SEQ ID NO: 4038)
CAGguacauc,
(SEQ ID NO: 1403)
CAGgugggac,
(SEQ ID NO: 727)
AGGguaaaua,
(SEQ ID NO: 4039)
UAAguaauua,
(SEQ ID NO: 1132)
CAGguaaccg,
(SEQ ID NO: 461)
AAGguuugca,
(SEQ ID NO: 4040)
UAGgugguuu,
(SEQ ID NO: 1327)
CAGgugaccg,
(SEQ ID NO: 4041)
UGUguaagcu,
(SEQ ID NO: 2227)
GGAgugaguc,
(SEQ ID NO: 752)
AGGguaggag,
(SEQ ID NO: 802)
AGGgugggug,
(SEQ ID NO: 313)
AAGgucugag,
(SEQ ID NO: 4042)
GAUguaauau,
(SEQ ID NO: 4043)
GGGguaauua,
(SEQ ID NO: 2524)
UAGguaggua,
(SEQ ID NO: 1858)
GAGgcaagua,
(SEQ ID NO: 1889)
GAGguaagga,
(SEQ ID NO: 4044)
UAGguacuac,
(SEQ ID NO: 4045)
UCGgugggug,
(SEQ ID NO: 401)
AAGgugugga,
(SEQ ID NO: 1305)
CAGgucugcc,
(SEQ ID NO: 4046)
UAAgugagcc,
(SEQ ID NO: 1820)
GAAguaaguu,
(SEQ ID NO: 1815)
GAAguaagcc,
(SEQ ID NO: 4047)
UAGgugcgac,
(SEQ ID NO: 4048)
GAGguauggc,
(SEQ ID NO: 2145)
GCAguaagaa,
(SEQ ID NO: 1438)
CAGgugugga,
(SEQ ID NO: 4049)
UUGguaacgu,
(SEQ ID NO: 4050)
GCUguaaaaa,
(SEQ ID NO: 4051)
UUGguuagua,
(SEQ ID NO: 4052)
AUAguaaggg,
(SEQ ID NO: 4053)
UUGguacuag,
(SEQ ID NO: 4054)
CGGgcagccg,
(SEQ ID NO: 1389)
CAGgugcugg,
(SEQ ID NO: 2673)
UAUgugaguu,
(SEQ ID NO: 4055)
CAGgucuggg,
(SEQ ID NO: 2415)
UAAguaagaa,
(SEQ ID NO: 4056)
AAGguuauua,
(SEQ ID NO: 4057)
AGAguaaagc,
(SEQ ID NO: 4058)
AGAgugugag,
(SEQ ID NO: 4059)
UAGgugcgag,
(SEQ ID NO: 4060)
CAAguaaacg,
(SEQ ID NO: 4061)
AAGguacgua,
(SEQ ID NO: 1759)
CUGgugagua,
(SEQ ID NO: 4062)
CCAguaugua,
(SEQ ID NO: 3006)
UUGgugagug,
(SEQ ID NO: 2772)
UGAguaagua,
(SEQ ID NO: 4063)
GAGguuagca,
(SEQ ID NO: 4064)
GUGguaagcc,
(SEQ ID NO: 1734)
CUGguauggc,
(SEQ ID NO: 8)
AAAguaacac,
(SEQ ID NO: 1186)
CAGguacuaa,
(SEQ ID NO: 2747)
UCUguaaguu,
(SEQ ID NO: 2024)
GAGgugaggg,
(SEQ ID NO: 647)
ACUgugggua,
(SEQ ID NO: 4065)
GAUguuugug,
(SEQ ID NO: 4066)
CAGgugucaa,
(SEQ ID NO: 4067)
CAGgucacca,
(SEQ ID NO: 4068)
CCGgugagua,
(SEQ ID NO: 4069)
UUGguaaaua,
(SEQ ID NO: 1411)
CAGguggggg,
(SEQ ID NO: 4070)
ACUgcaggug,
(SEQ ID NO: 2554)
UAGguauguu,
(SEQ ID NO: 4071)
GGAgcaagug,
(SEQ ID NO: 4072)
UCGgugccuc,
(SEQ ID NO: 4073)
CAAguaacuu,
(SEQ ID NO: 1879)
GAGguaacca,
(SEQ ID NO: 1151)
CAGguaauau,
(SEQ ID NO: 4074)
GGAguaagaa,
(SEQ ID NO: 1914)
GAGguaccuu,
(SEQ ID NO: 737)
AGGguaagga,
(SEQ ID NO: 1609)
CCUgugaguc,
(SEQ ID NO: 1900)
GAGguaaugg,
(SEQ ID NO: 4075)
AUGguguguc,
(SEQ ID NO: 4076)
GGGgugagua,
(SEQ ID NO: 4077)
AGGgucaggu,
(SEQ ID NO: 2839)
UGGguaaggg,
(SEQ ID NO: 759)
AGGguagguu,
(SEQ ID NO: 4078)
AUAgugaguu,
(SEQ ID NO: 4079)
CCCguaggcu,
(SEQ ID NO: 553)
ACAguaugua,
(SEQ ID NO: 4080)
GACgugugua,
(SEQ ID NO: 4081)
GCGgugagga,
(SEQ ID NO: 1326)
CAGgugaccc,
(SEQ ID NO: 4082)
UAAguuuagu,
(SEQ ID NO: 570)
ACAguugagu,
(SEQ ID NO: 1639)
CGGgugaggg,
(SEQ ID NO: 1398)
CAGguggauu,
(SEQ ID NO: 4083)
CGGguagagg,
(SEQ ID NO: 2608)
UAGgugcgug,
(SEQ ID NO: 2243)
GGGguaagaa,
(SEQ ID NO: 4084)
GAGguggggu,
(SEQ ID NO: 4085)
CACguggguu,
(SEQ ID NO: 4086)
ACGguaauug,
(SEQ ID NO: 705)
AGAgugaguc,
(SEQ ID NO: 4087)
UUGgcuccaa,
(SEQ ID NO: 355)
AAGgugaugc,
(SEQ ID NO: 448)
AAGguugguc,
(SEQ ID NO: 4088)
AGCguaaguu,
(SEQ ID NO: 1006)
AUUguaugua,
(SEQ ID NO: 4089)
UCAguuaagu,
(SEQ ID NO: 4090)
CAAguacgug,
(SEQ ID NO: 1382)
CAGgugcgug,
(SEQ ID NO: 1220)
CAGguaggua,
(SEQ ID NO: 4091)
AUGguggggu,
(SEQ ID NO: 964)
AUGgugaguu,
(SEQ ID NO: 4092)
CAGguaauca,
(SEQ ID NO: 226)
AAGguagggu,
(SEQ ID NO: 4093)
CAGgccaagg,
(SEQ ID NO: 4094)
GUGgugagag,
(SEQ ID NO: 449)
AAGguuggug,
(SEQ ID NO: 1190)
CAGguacucu,
(SEQ ID NO: 4095)
UAGgcaugug,
(SEQ ID NO: 4096)
UUGguaccuu,
(SEQ ID NO: 4097)
CUGgugugcc,
(SEQ ID NO: 4098)
ACAguugcca,
(SEQ ID NO: 4099)
UUGguaauau,
(SEQ ID NO: 4100)
GAGgugcaug,
(SEQ ID NO: 3028)
UUGguuugua,
(SEQ ID NO: 2963)
UUGguaagug,
(SEQ ID NO: 4101)
UGUgugugug,
(SEQ ID NO: 2398)
GUGguuugua,
(SEQ ID NO: 4102)
GCGguacaca,
(SEQ ID NO: 4103)
AGAguaugcu,
(SEQ ID NO: 3038)
UUUguaagua,
(SEQ ID NO: 4104)
UCUgugcggg,
(SEQ ID NO: 295)
AAGgucagug,
(SEQ ID NO: 1930)
GAGguaggaa,
(SEQ ID NO: 4105)
GCGguuagca,
(SEQ ID NO: 793)
AGGgugaggg,
(SEQ ID NO: 4106)
GAAgugagua,
(SEQ ID NO: 4107)
CAGgugacag,
(SEQ ID NO: 357)
AAGgugauua,
(SEQ ID NO: 4108)
GAGgccagcc,
(SEQ ID NO: 4109)
GAGgucuccu,
(SEQ ID NO: 2556)
UAGguauuac,
(SEQ ID NO: 1519)
CAUguaagag,
(SEQ ID NO: 4110)
CUGguagggc,
(SEQ ID NO: 1818)
GAAguaagua,
(SEQ ID NO: 4111)
CGGguaagug,
(SEQ ID NO: 4112)
CAGguaaucu,
(SEQ ID NO: 4113)
GUGguaggua,
(SEQ ID NO: 1413)
CAGgugggua,
(SEQ ID NO: 4114)
AAGgccagug,
(SEQ ID NO: 4115)
AAAgugaauc,
(SEQ ID NO: 4116)
ACGguuacgu,
(SEQ ID NO: 917)
AUGguaggaa,
(SEQ ID NO: 4117)
CGGgugagac,
(SEQ ID NO: 2099)
GAGguuggaa,
(SEQ ID NO: 2871)
UGGgugagcc,
(SEQ ID NO: 1564)
CCAgugagua,
(SEQ ID NO: 4118)
CUAguacgag,
(SEQ ID NO: 1248)
CAGguaugac,
(SEQ ID NO: 4119)
GCUgugaggu,
(SEQ ID NO: 4120)
CUGguaugaa,
(SEQ ID NO: 4121)
GGUguacgac,
(SEQ ID NO: 4122)
CUUgugagug,
(SEQ ID NO: 2380)
GUGgugagca,
(SEQ ID NO: 1696)
CUGguaacuu,
(SEQ ID NO: 1188)
CAGguacuau,
(SEQ ID NO: 739)
AGGguaaggg,
(SEQ ID NO: 3025)
UUGguuaguu,
(SEQ ID NO: 2302)
GGUguaagca,
(SEQ ID NO: 4123)
UCGgugagga,
(SEQ ID NO: 4124)
UGGguaaaca,
(SEQ ID NO: 4125)
UCGguacgug,
(SEQ ID NO: 4126)
UAGguagcag,
(SEQ ID NO: 1704)
CUGguaaggc,
(SEQ ID NO: 2349)
GUGguaagga,
(SEQ ID NO: 2418)
UAAguaagca,
(SEQ ID NO: 4127)
GAGguuccaa,
(SEQ ID NO: 4128)
CUGguaugga,
(SEQ ID NO: 2288)
GGGgugggua,
(SEQ ID NO: 4129)
CAGguuuccc,
(SEQ ID NO: 4130)
CAGgucucug,
(SEQ ID NO: 2022)
GAGgugagga,
(SEQ ID NO: 1805)
CUUguggguu,
(SEQ ID NO: 953)
AUGgugagac,
(SEQ ID NO: 1319)
CAGgugaagg,
(SEQ ID NO: 4131)
GCGguagggg,
(SEQ ID NO: 4132)
GUUguuuccc,
(SEQ ID NO: 4133)
AAAgcaucca,
(SEQ ID NO: 2367)
GUGguagguu,
(SEQ ID NO: 398)
AAGgugugaa,
(SEQ ID NO: 1167)
CAGguacagu,
(SEQ ID NO: 182)
AAGguaccaa,
(SEQ ID NO: 2969)
UUGguaauug,
(SEQ ID NO: 4134)
AAGgugcuca,
(SEQ ID NO: 4135)
AAGguucaac,
(SEQ ID NO: 4136)
CAGguuuaca,
(SEQ ID NO: 2195)
GCUguaagug,
(SEQ ID NO: 769)
AGGguauguc,
(SEQ ID NO: 1996)
GAGgucgggg,
(SEQ ID NO: 363)
AAGgugccug,
(SEQ ID NO: 119)
AAGguaaaaa,
(SEQ ID NO: 2385)
GUGgugaguu,
(SEQ ID NO: 4137)
UAGguaagaa,
(SEQ ID NO: 4138)
AGGguauccu,
(SEQ ID NO: 4139)
GUGguaauau,
(SEQ ID NO: 2744)
UCUguaagua,
(SEQ ID NO: 4140)
UGGguaugga,
(SEQ ID NO: 935)
AUGguaugga,
(SEQ ID NO: 1854)
GACgugagcc,
(SEQ ID NO: 4141)
CUGguuuggc,
(SEQ ID NO: 4142)
AUGguauauc,
(SEQ ID NO: 4143)
AAAguaaacu,
(SEQ ID NO: 721)
AGCgugagug,
(SEQ ID NO: 4144)
CUGguauaga,
(SEQ ID NO: 1409)
CAGgugggga,
(SEQ ID NO: 696)
AGAguauguu,
(SEQ ID NO: 4145)
UAGguacuug,
(SEQ ID NO: 4146)
GCAguaggug,
(SEQ ID NO: 4147)
AGUguauguc,
(SEQ ID NO: 413)
AAGguuaagc,
(SEQ ID NO: 4148)
CUGguggccu,
(SEQ ID NO: 1839)
GAAgugaguc,
(SEQ ID NO: 4149)
UUGguguaag,
(SEQ ID NO: 1138)
CAGguaagaa,
(SEQ ID NO: 4150)
CGGgucucgg,
(SEQ ID NO: 2035)
GAGgugcaca,
(SEQ ID NO: 4151)
CUCguuaguu,
(SEQ ID NO: 352)
AAGgugauca,
(SEQ ID NO: 2649)
UAUguaagaa,
(SEQ ID NO: 2047)
GAGgugcuug,
(SEQ ID NO: 4152)
CAGgugguca,
(SEQ ID NO: 4153)
ACGguaaguc,
(SEQ ID NO: 4154)
ACAguaaugu,
(SEQ ID NO: 4155)
CCUguaaggu,
(SEQ ID NO: 4156)
GAGguuaagu,
(SEQ ID NO: 2725)
UCGguaugug,
(SEQ ID NO: 2863)
UGGguauguu,
(SEQ ID NO: 268)
AAGguauuac,
(SEQ ID NO: 1343)
CAGgugaggg,
(SEQ ID NO: 4157)
UUGguaaaca,
(SEQ ID NO: 4158)
AAGguagugu,
(SEQ ID NO: 4159)
GAGguguggc,
(SEQ ID NO: 4160)
CAGguacgga,
(SEQ ID NO: 4161)
AAGgucauca,
(SEQ ID NO: 4162)
CAAguaggca,
(SEQ ID NO: 4163)
CAGgugaaac,
(SEQ ID NO: 1192)
CAGguacugc,
(SEQ ID NO: 4164)
AAUgcaagug,
(SEQ ID NO: 4165)
CAUguaauuc,
(SEQ ID NO: 259)
AAGguaugcu,
(SEQ ID NO: 1762)
CUGgugaguu,
(SEQ ID NO: 4166)
CAGgugguuu,
(SEQ ID NO: 2922)
UGUgugagua,
(SEQ ID NO: 4167)
AAGgucggug,
(SEQ ID NO: 883)
AUGguaaauu,
(SEQ ID NO: 771)
AGGguauuac,
(SEQ ID NO: 4168)
AGUguaugga,
(SEQ ID NO: 4169)
AACguaagau,
(SEQ ID NO: 4170)
GUGguaaggu,
(SEQ ID NO: 4171)
ACUguuagua,
(SEQ ID NO: 1239)
CAGguaucag,
(SEQ ID NO: 425)
AAGguuaguu,
(SEQ ID NO: 1754)
CUGgugagcu,
(SEQ ID NO: 4172)
UUGgugagcu,
(SEQ ID NO: 4173)
UGUguacgua,
(SEQ ID NO: 4174)
GAGgucagcc,
(SEQ ID NO: 4175)
GAGguagaau,
(SEQ ID NO: 255)
AAGguaugag,
(SEQ ID NO: 2563)
UAGguauuuc,
(SEQ ID NO: 4176)
UGUguaacac,
(SEQ ID NO: 4177)
AGUguaaggc,
(SEQ ID NO: 4178)
GAGgucugcu,
(SEQ ID NO: 418)
AAGguuagca,
(SEQ ID NO: 1127)
CAGguaaaug,
(SEQ ID NO: 4179)
AACguaagcu,
(SEQ ID NO: 4180)
CAGgucugca,
(SEQ ID NO: 1267)
CAGguauugu,
(SEQ ID NO: 2356)
GUGguaauuc,
(SEQ ID NO: 1951)
GAGguauaug,
(SEQ ID NO: 4181)
GCCgugagcc,
(SEQ ID NO: 1883)
GAGguaagag,
(SEQ ID NO: 2787)
UGAguaugua,
(SEQ ID NO: 1145)
CAGguaaggg,
(SEQ ID NO: 1876)
GAGguaaauu,
(SEQ ID NO: 4182)
CAGgcaacuu,
(SEQ ID NO: 2908)
UGUguaaguc,
(SEQ ID NO: 4183)
CAGgugcgcu,
(SEQ ID NO: 4184)
CGGguaaacc,
(SEQ ID NO: 4185)
CCGgucaguc,
(SEQ ID NO: 4186)
UAGgugggcg,
(SEQ ID NO: 4187)
GCGgucaguu,
(SEQ ID NO: 4188)
GGGguggguc,
(SEQ ID NO: 4189)
AGCguaauag,
(SEQ ID NO: 4190)
ACGgugaguc,
(SEQ ID NO: 1722)
CUGguacuug,
(SEQ ID NO: 4191)
CAGguuggua,
(SEQ ID NO: 695)
AGAguaugug,
(SEQ ID NO: 1771)
CUGgugggua,
(SEQ ID NO: 4192)
GAGguggcuu,
(SEQ ID NO: 4193)
AUAguauuga,
(SEQ ID NO: 4194)
UGAgucgucc,
(SEQ ID NO: 4195)
CAGgugcucu,
(SEQ ID NO: 4196)
UACguaauau,
(SEQ ID NO: 4197)
GCUguccuga,
(SEQ ID NO: 4198)
CAGgcugcac,
(SEQ ID NO: 1766)
CUGgugcgcu,
(SEQ ID NO: 4199)
GCGguaagaa,
(SEQ ID NO: 4200)
UAAguuacuu,
(SEQ ID NO: 1840)
GAAgugagug,
(SEQ ID NO: 2460)
UAGgcaaguc,
(SEQ ID NO: 4201)
UAAguaaaua,
(SEQ ID NO: 607)
ACGgugagug,
(SEQ ID NO: 1223)
CAGguagguu,
(SEQ ID NO: 4202)
GGGguauaac,
(SEQ ID NO: 2410)
GUUgugaguu,
(SEQ ID NO: 1539)
CAUgugagua,
(SEQ ID NO: 4203)
GAGgugcauu,
(SEQ ID NO: 466)
AAGguuugua,
(SEQ ID NO: 4204)
UCGguaaugu,
(SEQ ID NO: 1616)
CGAguaaggg,
(SEQ ID NO: 4205)
GAGgcacgga,
(SEQ ID NO: 4206)
AGGgugugga,
(SEQ ID NO: 1257)
CAGguauggu,
(SEQ ID NO: 203)
AAGguagaaa,
(SEQ ID NO: 1373)
CAGgugccug,
(SEQ ID NO: 4207)
UGGguauaug,
(SEQ ID NO: 4208)
UGAgugagac,
(SEQ ID NO: 2847)
UGGguaauuu,
(SEQ ID NO: 881)
AUGguaaaua,
(SEQ ID NO: 4209)
AAGgcaaagg,
(SEQ ID NO: 4210)
AGUguuuguu,
(SEQ ID NO: 4211)
AUGguauugg,
(SEQ ID NO: 1756)
CUGgugaggc,
(SEQ ID NO: 2948)
UUGguaaaau,
(SEQ ID NO: 563)
ACAgugaguu,
(SEQ ID NO: 4212)
CAGgugcugu,
(SEQ ID NO: 2080)
GAGguuaaga,
(SEQ ID NO: 659)
AGAguaagaa,
(SEQ ID NO: 4213)
GAGguccgcg,
(SEQ ID NO: 2382)
GUGgugagga,
(SEQ ID NO: 1338)
CAGgugagcc,
(SEQ ID NO: 1324)
CAGgugacau,
(SEQ ID NO: 4214)
AUGgcaagcu,
(SEQ ID NO: 4215)
UCGguaauau,
(SEQ ID NO: 4216)
CAGgcaacaa,
(SEQ ID NO: 2257)
GGGguaggga,
(SEQ ID NO: 4217)
CUGgucucgc,
(SEQ ID NO: 4218)
UAGguaacga,
(SEQ ID NO: 4219)
CGGguaaggu,
(SEQ ID NO: 4220)
UAGguaaugc,
(SEQ ID NO: 1099)
CAGgcaagaa,
(SEQ ID NO: 4221)
ACAguaggua,
(SEQ ID NO: 1049)
CAAguaugag,
(SEQ ID NO: 4222)
GCUguucgaa,
(SEQ ID NO: 4223)
AAGguuaugc,
(SEQ ID NO: 2136)
GAUgugaguu,
(SEQ ID NO: 1396)
CAGguggaga,
(SEQ ID NO: 4224)
AGAguuaguu,
(SEQ ID NO: 4225)
UGAgugugcg,
(SEQ ID NO: 1907)
GAGguacagc,
(SEQ ID NO: 1139)
CAGguaagac,
(SEQ ID NO: 4226)
CAUgugcuuu,
(SEQ ID NO: 4227)
AGGguguguu,
(SEQ ID NO: 4228)
ACAguuaagg,
(SEQ ID NO: 4229)
ACAgugaggg,
(SEQ ID NO: 4230)
GAUguauacc,
(SEQ ID NO: 4231)
UUAguaagcu,
(SEQ ID NO: 1141)
CAGguaagau,
(SEQ ID NO: 4232)
AGAgcugcgu,
(SEQ ID NO: 1860)
GAGgcaaguu,
(SEQ ID NO: 1819)
GAAguaagug,
(SEQ ID NO: 4233)
AAGgugaaaa,
(SEQ ID NO: 4234)
AAGguaccua,
(SEQ ID NO: 4235)
GAGguaucag,
(SEQ ID NO: 4236)
AUGguaugua,
(SEQ ID NO: 253)
AAGguaugaa,
(SEQ ID NO: 4237)
UUGgugagcc,
(SEQ ID NO: 420)
AAGguuagga,
(SEQ ID NO: 768)
AGGguaugua,
(SEQ ID NO: 4238)
CAGguaccga,
(SEQ ID NO: 4239)
AGAguaaacu,
(SEQ ID NO: 4240)
AAGgugcaua,
(SEQ ID NO: 167)
AAGguaaugu,
(SEQ ID NO: 4241)
CCGgugugug,
(SEQ ID NO: 729)
AGGguaaauu,
(SEQ ID NO: 4242)
GGGguuuggc,
(SEQ ID NO: 1164)
CAGguacacg,
(SEQ ID NO: 4243)
UUGguaacca,
(SEQ ID NO: 1986)
GAGgucaggu,
(SEQ ID NO: 4244)
UCUguuggua,
(SEQ ID NO: 1458)
CAGguuaguu,
(SEQ ID NO: 4245)
UUGguauguc,
(SEQ ID NO: 4246)
AAGgugcguc,
(SEQ ID NO: 733)
AGGguaagaa,
(SEQ ID NO: 4247)
UUUguaagcc,
(SEQ ID NO: 292)
AAGgucaggu,
(SEQ ID NO: 4248)
CUGguaaacu,
(SEQ ID NO: 4249)
UCGguaauuu,
(SEQ ID NO: 4250)
CUGguaggcu,
(SEQ ID NO: 4251)
GAGgucugua,
(SEQ ID NO: 1922)
GAGguacuuu,
(SEQ ID NO: 4252)
CUGguaaagg,
(SEQ ID NO: 1650)
CGGgugugug,
(SEQ ID NO: 4253)
CAGguguggu,
(SEQ ID NO: 4254)
UCGguacguc,
(SEQ ID NO: 4255)
CAGgugccag,
(SEQ ID NO: 2275)
GGGgugagaa,
(SEQ ID NO: 4256)
ACAgcuagua,
(SEQ ID NO: 4257)
AAGguauagc,
(SEQ ID NO: 4258)
CUGguaggag,
(SEQ ID NO: 4259)
GCUguacgua,
(SEQ ID NO: 128)
AAGguaaagg,
(SEQ ID NO: 4260)
CAAgcacgag,
(SEQ ID NO: 4261)
CUAguaagac,
(SEQ ID NO: 4262)
CCCguaagcg,
(SEQ ID NO: 1078)
CAAgugugag,
(SEQ ID NO: 897)
AUGguaaggg,
(SEQ ID NO: 345)
AAGgugaggg,
(SEQ ID NO: 1041)
CAAguaggua,
(SEQ ID NO: 2321)
GGUguugcug,
(SEQ ID NO: 1920)
GAGguacugu,
(SEQ ID NO: 2484)
UAGguaagau,
(SEQ ID NO: 1374)
CAGgugcgaa,
(SEQ ID NO: 4263)
GAGguccagg,
(SEQ ID NO: 2982)
UUGguauaca,
(SEQ ID NO: 2226)
GGAgugagua,
(SEQ ID NO: 2017)
GAGgugagau,
(SEQ ID NO: 4264)
AAGguggggc,
(SEQ ID NO: 4265)
CAGguaaacg,
(SEQ ID NO: 4266)
UCGguaacuu,
(SEQ ID NO: 1128)
CAGguaaauu,
(SEQ ID NO: 4267)
GAGgugcgca,
(SEQ ID NO: 643)
ACUgugagua,
(SEQ ID NO: 4268)
ACGgugugac,
(SEQ ID NO: 2352)
GUGguaaguc,
(SEQ ID NO: 1215)
CAGguaggca,
(SEQ ID NO: 1277)
CAGgucagca,
(SEQ ID NO: 4269)
GUGguaugug,
(SEQ ID NO: 4270)
AAAguaucug,
(SEQ ID NO: 4271)
CGGguaugua,
(SEQ ID NO: 157)
AAGguaauaa,
(SEQ ID NO: 2060)
GAGgugggga,
(SEQ ID NO: 2197)
GCUguaggug,
(SEQ ID NO: 1841)
GAAgugaguu,
(SEQ ID NO: 4272)
AAAguauuua,
(SEQ ID NO: 2653)
UAUguaagua,
(SEQ ID NO: 4273)
ACGguaugag,
(SEQ ID NO: 1761)
CUGgugagug,
(SEQ ID NO: 4274)
AGAguaaaau,
(SEQ ID NO: 4275)
GCUguauggc,
(SEQ ID NO: 879)
AUGguaaacc,
(SEQ ID NO: 4276)
GCAguaauaa,
(SEQ ID NO: 4277)
UAAguauuua,
(SEQ ID NO: 515)
AAUgucagug,
(SEQ ID NO: 4278)
AUUgcaggag,
(SEQ ID NO: 4279)
CCGguaagaa,
(SEQ ID NO: 101)
AAGgcaaguu,
(SEQ ID NO: 4280)
GAGguuuguc,
(SEQ ID NO: 139)
AAGguaacug,
(SEQ ID NO: 4281)
AAAguaugag,
(SEQ ID NO: 4282)
GAUguuagua,
(SEQ ID NO: 1414)
CAGguggguc,
(SEQ ID NO: 4283)
AAGguaccga,
(SEQ ID NO: 4284)
CCAguaauua,
(SEQ ID NO: 4285)
GUGguaugcg,
(SEQ ID NO: 4286)
AUGgugcgcu,
(SEQ ID NO: 4287)
CAGgucuaug,
(SEQ ID NO: 274)
AAGguauuua,
(SEQ ID NO: 4288)
CUAguaagau,
(SEQ ID NO: 675)
AGAguaauuu,
(SEQ ID NO: 4289)
GAGguaacgu,
(SEQ ID NO: 212)
AAGguagcca,
(SEQ ID NO: 4290)
CUGgucccgg,
(SEQ ID NO: 4291)
GAGguccuuc,
(SEQ ID NO: 4292)
ACGgucaccc,
(SEQ ID NO: 158)
AAGguaauac,
(SEQ ID NO: 1367)
CAGgugcaug,
(SEQ ID NO: 4293)
AUGguaauag,
(SEQ ID NO: 4294)
UUUguaacac,
(SEQ ID NO: 4295)
UGGguaugau,
(SEQ ID NO: 4296)
CAGgcccccc,
(SEQ ID NO: 4297)
AGAguaguaa,
(SEQ ID NO: 814)
AGUguaagaa,
(SEQ ID NO: 1833)
GAAguauguu,
(SEQ ID NO: 1434)
CAGgugugca,
(SEQ ID NO: 3003)
UUGgugaggg,
(SEQ ID NO: 4298)
UGGguugguu,
(SEQ ID NO: 1184)
CAGguacgua,
(SEQ ID NO: 4299)
GAGgugcggc,
(SEQ ID NO: 4300)
UCUguacggg,
(SEQ ID NO: 4301)
CGGgugcgug,
(SEQ ID NO: 2455)
UACguaagug,
(SEQ ID NO: 4302)
CAUguaagga,
(SEQ ID NO: 1329)
CAGgugacgg,
(SEQ ID NO: 4303)
GAUguaugcu,
(SEQ ID NO: 4304)
UCUgcaauuc,
(SEQ ID NO: 2770)
UGAguaaggc,
(SEQ ID NO: 1952)
GAGguauauu,
(SEQ ID NO: 707)
AGAgugaguu,
(SEQ ID NO: 148)
AAGguaagcu,
(SEQ ID NO: 2580)
UAGgugaagu,
(SEQ ID NO: 1455)
CAGguuagua,
(SEQ ID NO: 2655)
UAUguaagug,
(SEQ ID NO: 4305)
UUGguggggg,
(SEQ ID NO: 4306)
UGAgcucaaa,
(SEQ ID NO: 4307)
UCGguaugua,
(SEQ ID NO: 4308)
UAAguaugcc,
(SEQ ID NO: 489)
AAUguaagua,
(SEQ ID NO: 4309)
CAGguuugca,
(SEQ ID NO: 4310)
ACGgugagag,
(SEQ ID NO: 4311)
CAGguguuuu,
(SEQ ID NO: 4312)
GUGgugagcc,
(SEQ ID NO: 4313)
AGGguacaua,
(SEQ ID NO: 4314)
UAGguaaccc,
(SEQ ID NO: 4315)
GUGgucagua,
(SEQ ID NO: 4316)
CUGgugagcc,
(SEQ ID NO: 1390)
CAGgugcuua,
(SEQ ID NO: 4317)
AUAgucguga,
(SEQ ID NO: 862)
AUAgugagug,
(SEQ ID NO: 4318)
GAGgucaaaa,
(SEQ ID NO: 4319)
CGUguagcuu,
(SEQ ID NO: 4320)
CAGguguuug,
(SEQ ID NO: 4321)
CAGguuggac,
(SEQ ID NO: 4322)
CAGguaagcu,
(SEQ ID NO: 4323)
AGGgucagaa,
(SEQ ID NO: 4324)
CACguauguc,
(SEQ ID NO: 1098)
CACgugagug,
(SEQ ID NO: 4325)
GGGguacgga,
(SEQ ID NO: 4326)
AAGgcaggac,
(SEQ ID NO: 4327)
GAGgugaagc,
(SEQ ID NO: 4328)
GAGguuugaa,
(SEQ ID NO: 1148)
CAGguaagug,
(SEQ ID NO: 1131)
CAGguaacca,
(SEQ ID NO: 1189),
CAGguacucc
(SEQ ID NO: 371)
AAGgugcuuu,
(SEQ ID NO: 1873)
GAGguaaaua,
(SEQ ID NO: 4329)
GAGgcaggug,
(SEQ ID NO: 4330)
GAGguucgga,
(SEQ ID NO: 1270)
CAGguauuug,
(SEQ ID NO: 1125)
CAGguaaaua,
(SEQ ID NO: 1354)
CAGgugaugu,
(SEQ ID NO: 4331)
CAGgugauac,
(SEQ ID NO: 2023)
GAGgugaggc,
(SEQ ID NO: 4332)
AGGguggggg,
(SEQ ID NO: 2425)
UAAguaaguu,
(SEQ ID NO: 4333)
UGGgugaaca,
(SEQ ID NO: 4334)
UAGguacugc,
(SEQ ID NO: 4335)
CAGgcuccug,
(SEQ ID NO: 753)
AGGguaggca,
(SEQ ID NO: 1371)
CAGgugcccg,
(SEQ ID NO: 4336)
GAGguacauc,
(SEQ ID NO: 804)
AGGgugugug,
(SEQ ID NO: 231)
AAGguaguaa,
(SEQ ID NO: 2859)
UGGguaugag,
(SEQ ID NO: 2294)
GGGgugugug,
(SEQ ID NO: 4337)
CUAguaggug,
(SEQ ID NO: 4338)
GAGgcaagga,
(SEQ ID NO: 4339)
AAGgcaagac,
(SEQ ID NO: 4340)
AAAgugcggu,
(SEQ ID NO: 450)
AAGguugguu,
(SEQ ID NO: 4341)
GAGguuaaug,
(SEQ ID NO: 3005)
UUGgugaguc,
(SEQ ID NO: 2738)
UCGguuagcu,
(SEQ ID NO: 4342)
GCAguaagca,
(SEQ ID NO: 4343)
AAGgcaagca,
(SEQ ID NO: 4344)
ACAguaagcu,
(SEQ ID NO: 1878)
GAGguaacag,
(SEQ ID NO: 4345)
AAAguacgua,
(SEQ ID NO: 1896)
GAGguaauac,
(SEQ ID NO: 2980)
UUGguaggug,
(SEQ ID NO: 4346)
CUGguuaguc,
(SEQ ID NO: 4347)
GAGgugacgc,
(SEQ ID NO: 4348)
ACAguaagga,
(SEQ ID NO: 4349)
AAUguacuua,
(SEQ ID NO: 4350)
GGGguacagu,
(SEQ ID NO: 4351)
CGUguaugug,
(SEQ ID NO: 4352)
UCCguagguu,
(SEQ ID NO: 4353)
GAGguggucg,
(SEQ ID NO: 4354)
UCAgugaguc,
(SEQ ID NO: 15)
AAAguaagca,
(SEQ ID NO: 1999)
GAGgucuggu,
(SEQ ID NO: 4355)
GAGguaauua,
(SEQ ID NO: 2323)
GUAguaagua,
(SEQ ID NO: 382)
AAGgugggga,
(SEQ ID NO: 4356)
UCUgugagca,
(SEQ ID NO: 4357)
GAAguucgug,
(SEQ ID NO: 4358)
ACGgugaggc,
(SEQ ID NO: 2699)
UCAgugagua,
(SEQ ID NO: 4359)
UAGguaguug,
(SEQ ID NO: 4360)
GGUgucuggg,
(SEQ ID NO: 2252)
GGGguaagug,
(SEQ ID NO: 2066)
GAGguggguu,
(SEQ ID NO: 4361)
UGUgugaguu,
(SEQ ID NO: 1522)
CAUguaagua,
(SEQ ID NO: 229)
AAGguaggug,
(SEQ ID NO: 4362)
AAUguaggag,
(SEQ ID NO: 4363)
GAGgcacguc,
(SEQ ID NO: 4364)
CAAguacauu,
(SEQ ID NO: 4365)
UUGguacaga,
(SEQ ID NO: 1941)
GAGguaguag,
(SEQ ID NO: 57)
AAAgugaggg,
(SEQ ID NO: 4366)
UUGgucagug,
(SEQ ID NO: 796)
AGGgugaguc,
(SEQ ID NO: 1317)
CAGgugaaca,
(SEQ ID NO: 4367)
GGUgugggcc,
(SEQ ID NO: 4368)
CGGgugagcu,
(SEQ ID NO: 2283)
GGGgugaguc,
(SEQ ID NO: 4369)
ACAgugagag,
(SEQ ID NO: 794)
AGGgugaggu,
(SEQ ID NO: 2194)
GCUguaaguc,
(SEQ ID NO: 4370)
AUAguagguu,
(SEQ ID NO: 1114)
CAGgcaugug,
(SEQ ID NO: 156)
AAGguaaguu,
(SEQ ID NO: 4371)
CAGguccgug,
(SEQ ID NO: 4372)
GAGgcaggua,
(SEQ ID NO: 4373)
AUGguggaag,
(SEQ ID NO: 4374)
AUGgugggcg,
(SEQ ID NO: 2014)
GAGgugagaa,
(SEQ ID NO: 832)
AGUgugagca,
(SEQ ID NO: 2962)
UUGguaagua,
(SEQ ID NO: 4375)
CAAguaagca,
(SEQ ID NO: 2313)
GGUgugagcu,
(SEQ ID NO: 4376)
CCCgugggua,
(SEQ ID NO: 4377)
CAGguagaau,
(SEQ ID NO: 4378)
CAGgcugagc,
(SEQ ID NO: 4379)
CUGguggccc,
(SEQ ID NO: 4380)
UGAguaagag,
(SEQ ID NO: 4381)
CACguuagcu,
(SEQ ID NO: 348)
AAGgugaguc,
(SEQ ID NO: 4382)
AAGguagcuc,
(SEQ ID NO: 4383)
UCGgugaguu,
(SEQ ID NO: 4384)
GAGgcccuuc,
(SEQ ID NO: 4385)
CAGguuaugc,
(SEQ ID NO: 4386)
CCUguaagcu,
(SEQ ID NO: 4387)
CAGgucuccu,
(SEQ ID NO: 4388)
UAGguaggcu,
(SEQ ID NO: 4389)
GGGguagggg,
(SEQ ID NO: 4390)
AAGguaguga,
(SEQ ID NO: 4391)
GAGguuguug,
(SEQ ID NO: 1489)
CAGguugguu,
(SEQ ID NO: 16)
AAAguaagcc,
(SEQ ID NO: 562)
ACAgugagug,
(SEQ ID NO: 4392)
UGGgugugau,
(SEQ ID NO: 4393)
CCCguaacua,
(SEQ ID NO: 408)
AAGguguugc,
(SEQ ID NO: 4394)
AAAgcuggug,
(SEQ ID NO: 4395)
GAGguauagu,
(SEQ ID NO: 4396)
ACGguaagag,
(SEQ ID NO: 913)
AUGguacggu,
(SEQ ID NO: 4397)
GAGgccaguu,
(SEQ ID NO: 1960)
GAGguaugcg,
(SEQ ID NO: 4398)
UCGgugggag,
(SEQ ID NO: 372)
AAGguggaua,
(SEQ ID NO: 4399)
CCAguguggc,
(SEQ ID NO: 742)
AGGguaagug,
(SEQ ID NO: 4400)
UCUguagguc,
(SEQ ID NO: 1102)
CAGgcaagga,
(SEQ ID NO: 1628)
CGGguaauuu,
(SEQ ID NO: 1010)
AUUgugaguc,
(SEQ ID NO: 1121)
CAGguaaacc,
(SEQ ID NO: 4401)
AAGgucaauu,
(SEQ ID NO: 327)
AAGgugaaua,
(SEQ ID NO: 4402)
GUCguaagaa
(SEQ ID NO: 4403)
GCGguaaguc,
(SEQ ID NO: 4404)
CUGguagagc,
(SEQ ID NO: 4405)
GAGgucgguc,
(SEQ ID NO: 1120)
CAGguaaaca,
(SEQ ID NO: 98)
AAGgcaagga,
(SEQ ID NO: 4406)
CAGgucgucu,
(SEQ ID NO: 4407)
GGGguagggc,
(SEQ ID NO: 1721)
CUGguacuaa,
(SEQ ID NO: 1929)
GAGguagcug,
(SEQ ID NO: 4408)
CUUgucagcu,
(SEQ ID NO: 2489)
UAGguaaggc,
(SEQ ID NO: 4409)
CUGguauuac,
(SEQ ID NO: 4410)
UAAguacguc,
(SEQ ID NO: 146)
AAGguaagcc,
(SEQ ID NO: 4411)
ACGgugaaag,
(SEQ ID NO: 4412)
CCAgccaaua,
(SEQ ID NO: 4413)
CAGguuuguc,
(SEQ ID NO: 239)
AAGguauaau,
(SEQ ID NO: 4414)
AAGgucuuag,
(SEQ ID NO: 791)
AGGgugagcu,
(SEQ ID NO: 4415)
AAGguuaggg,
(SEQ ID NO: 4416)
CGGguaaauu,
(SEQ ID NO: 4417)
CAGguaacgg,
(SEQ ID NO: 4418)
AGAgugugua,
(SEQ ID NO: 549)
ACAguaaguu,
(SEQ ID NO: 4419)
GAUguaauuu,
(SEQ ID NO: 1934)
GAGguaggga,
(SEQ ID NO: 2945)
UUGgcaagug,
(SEQ ID NO: 4420)
AAAgugagga,
(SEQ ID NO: 234)
AAGguagugc,
(SEQ ID NO: 674)
AGAguaauuc,
(SEQ ID NO: 4421)
GGAguaaaua,
(SEQ ID NO: 4422)
GUGguaccca,
(SEQ ID NO: 4423)
CAGguauugc,
(SEQ ID NO: 4424)
GAUgugaggg,
(SEQ ID NO: 1017)
CAAguaaauc,
(SEQ ID NO: 1428)
CAGgugucuc,
(SEQ ID NO: 4425)
AAGguaacag,
(SEQ ID NO: 4426)
UUGguaaaag,
(SEQ ID NO: 1240)
CAGguaucau,
(SEQ ID NO: 4427)
ACGgugagac,
(SEQ ID NO: 4428)
CUGguaugac,
(SEQ ID NO: 4429)
CAGguucacu,
(SEQ ID NO: 4430)
GAGgugauca,
(SEQ ID NO: 4431)
AGUguaaguc,
(SEQ ID NO: 4432)
AACguaagua,
(SEQ ID NO: 60)
AAAgugagug,
(SEQ ID NO: 4433)
GAGguacagg,
(SEQ ID NO: 4434)
CAAguaauga,
(SEQ ID NO: 4435)
GAUguaagga,
(SEQ ID NO: 4436)
UCAguucccc,
(SEQ ID NO: 4437)
GCGguaagga,
(SEQ ID NO: 4438)
UAGguacuaa,
(SEQ ID NO: 321)
AAGgugaaag,
(SEQ ID NO: 4439)
ACUguaagug,
(SEQ ID NO: 2862)
UGGguaugug,
(SEQ ID NO: 884)
AUGguaacag,
(SEQ ID NO: 1219)
CAGguagggu,
(SEQ ID NO: 548)
ACAguaagug,
(SEQ ID NO: 366)
AAGgugcucc,
(SEQ ID NO: 4440)
AAGgugugcu,
(SEQ ID NO: 4441)
AAGgugguga,
(SEQ ID NO: 4442)
ACGgugcgcc,
(SEQ ID NO: 4443)
AAGguauugc,
(SEQ ID NO: 2267)
GGGguaugug,
(SEQ ID NO: 1408)
CAGgugggcu,
(SEQ ID NO: 1968)
GAGguauguu,
(SEQ ID NO: 4444)
AACgugaaua,
(SEQ ID NO: 1154)
CAGguaaugg,
(SEQ ID NO: 4445)
UAGguaugau,
(SEQ ID NO: 1108)
CAGgcaggug,
(SEQ ID NO: 4446)
GGGguugguc,
(SEQ ID NO: 262)
AAGguauggg,
(SEQ ID NO: 4447)
UAAgugaggc,
(SEQ ID NO: 4448)
CAAgugaucg,
(SEQ ID NO: 4449)
AAAguacggg,
(SEQ ID NO: 4450)
AGAgcuacag,
(SEQ ID NO: 2054)
GAGgugggaa,
(SEQ ID NO: 1195)
CAGguacuuu,
(SEQ ID NO: 2016)
GAGgugagag,
(SEQ ID NO: 1221)
CAGguagguc,
(SEQ ID NO: 4451)
UGGguacagc,
(SEQ ID NO: 396)
AAGgugucag,
(SEQ ID NO: 4452)
AAGgcaagaa,
(SEQ ID NO: 4453)
GAGguaaaca,
(SEQ ID NO: 129)
AAGguaaagu,
(SEQ ID NO: 4454)
AAGguaguca,
(SEQ ID NO: 4455)
CUGguauguc,
(SEQ ID NO: 1963)
GAGguauggg,
(SEQ ID NO: 273)
AAGguauugu,
(SEQ ID NO: 4456)
CUGguacuga,
(SEQ ID NO: 1888)
GAGguaagcu,
(SEQ ID NO: 2883)
UGGgugggua,
(SEQ ID NO: 4457)
CAGguucgug,
(SEQ ID NO: 4458)
AAGguauggu,
(SEQ ID NO: 1337)
CAGgugagca,
(SEQ ID NO: 2827)
UGGguaaauu,
(SEQ ID NO: 4459)
UGUguaggug,
(SEQ ID NO: 2921)
UGUgugagcc,
(SEQ ID NO: 4460)
CUGguaauau,
(SEQ ID NO: 45)
AAAguauguu,
(SEQ ID NO: 2903)
UGUguaagaa,
(SEQ ID NO: 4461)
CUAgugagaa,
(SEQ ID NO: 757)
AGGguagguc,
(SEQ ID NO: 385)
AAGgugggug,
(SEQ ID NO: 4462)
UCGguaagug,
(SEQ ID NO: 812)
AGUguaaaua,
(SEQ ID NO: 2122)
GAUguaagug,
(SEQ ID NO: 424)
AAGguuagug,
(SEQ ID NO: 2485)
UAGguaagca,
(SEQ ID NO: 1061)
CAAgugagaa,
(SEQ ID NO: 819)
AGUguaagua,
(SEQ ID NO: 1321)
CAGgugaauc,
(SEQ ID NO: 2868)
UGGgugagac,
(SEQ ID NO: 224)
AAGguagggc,
(SEQ ID NO: 1788)
CUGguuugug,
(SEQ ID NO: 4463)
GCGguagggc,
(SEQ ID NO: 4464)
GAGguaaucc,
(SEQ ID NO: 4465)
AUUguaauaa,
(SEQ ID NO: 1748)
CUGgugaaua,
(SEQ ID NO: 4466)
AAGguuuaaa,
(SEQ ID NO: 4467)
CCUguacugu,
(SEQ ID NO: 4468)
GCGgugagcg,
(SEQ ID NO: 162)
AAGguaaucc,
(SEQ ID NO: 2671)
UAUgugagua,
(SEQ ID NO: 1573)
CCCgugagug,
(SEQ ID NO: 1363)
CAGgugcaga,
(SEQ ID NO: 1284)
CAGgucaguu,
(SEQ ID NO: 4469)
CAGguaggcu,
(SEQ ID NO: 23)
AAAguaagug,
(SEQ ID NO: 4470)
UAGguugguc,
(SEQ ID NO: 4471)
CAGguugccu,
(SEQ ID NO: 260)
AAGguaugga,
(SEQ ID NO: 4472)
GGUguggacg,
(SEQ ID NO: 51)
AAAgugagaa,
(SEQ ID NO: 788)
AGGgugagag,
(SEQ ID NO: 4473)
GAUguggcau,
(SEQ ID NO: 4474)
UCGguaaggu,
(SEQ ID NO: 4475)
GAGgugcguc,
(SEQ ID NO: 4476)
CGGgugaguc,
(SEQ ID NO: 190)
AAGguacggg,
(SEQ ID NO: 4477)
GAGguucuug,
(SEQ ID NO: 4478)
AAGgugcuug,
(SEQ ID NO: 2551)
UAGguaugua,
(SEQ ID NO: 4479)
AUGgucagca,
(SEQ ID NO: 4480)
CGGguacuca,
(SEQ ID NO: 792)
AGGgugagga,
(SEQ ID NO: 869)
AUCgugagua,
(SEQ ID NO: 2689)
UCAguaagua,
(SEQ ID NO: 2469)
UAGguaaaua,
(SEQ ID NO: 170)
AAGguaaug,
(SEQ ID NO: 1835)
GAAgucagug,
(SEQ ID NO: 1160)
CAGguacaaa,
(SEQ ID NO: 4481)
AAAguuaauc,
(SEQ ID NO: 4482)
AGCgugagcg,
(SEQ ID NO: 4483)
CCGgcuggug,
(SEQ ID NO: 4484)
AGUguaauuu,
(SEQ ID NO: 4485)
UGAgccacuc,
(SEQ ID NO: 4486)
GGGgucugua,
(SEQ ID NO: 4487)
AUGgcauguc,
(SEQ ID NO: 4488)
CGGguaaaga,
(SEQ ID NO: 4489)
AGGguagcau,
(SEQ ID NO: 1631)
CGGguaggag,
(SEQ ID NO: 4490)
GAGguucgug,
(SEQ ID NO: 4491)
UAAguuauuc,
(SEQ ID NO: 2650)
UAUguaagau,
(SEQ ID NO: 237)
AAGguaguuu,
(SEQ ID NO: 4492)
CAGgugguau,
(SEQ ID NO: 2355)
GUGguaauga,
(SEQ ID NO: 359)
AAGgugauuu,
(SEQ ID NO: 4493)
CAGgugaagu,
(SEQ ID NO: 4494)
GUAguaauua,
(SEQ ID NO: 4495)
AUGguuggug,
(SEQ ID NO: 1557)
CCAguaagug,
(SEQ ID NO: 2589)
UAGgugagag,
(SEQ ID NO: 959)
AUGgugaggc,
(SEQ ID NO: 72)
AAAguuagug,
(SEQ ID NO: 4496)
AAGgugccuu,
(SEQ ID NO: 2546)
UAGguaugag,
(SEQ ID NO: 1431)
CAGgugugac,
(SEQ ID NO: 1774)
CUGguggguu,
(SEQ ID NO: 896)
AUGguaagga,
(SEQ ID NO: 2740)
UCUguaagaa,
(SEQ ID NO: 4497)
UCCgugaguu,
(SEQ ID NO: 4498)
AAAgcaggua,
(SEQ ID NO: 2672)
UAUgugagug,
(SEQ ID NO: 4499)
CAGguggagg,
(SEQ ID NO: 4500)
CAGguuagac,
(SEQ ID NO: 846)
AUAguaagac,
(SEQ ID NO: 4501)
AAGguguugu,
(SEQ ID NO: 4502)
GAGgucugug,
(SEQ ID NO: 144)
AAGguaagau,
(SEQ ID NO: 1524)
CAUguaaguu,
(SEQ ID NO: 4503)
CUGguaauua,
(SEQ ID NO: 4504)
CAGguaggcg,
(SEQ ID NO: 669)
AGAguaaguc,
(SEQ ID NO: 2872)
UGGgugagga,
(SEQ ID NO: 4505)
AAUguaggua,
(SEQ ID NO: 4506)
UAGguuagca,
(SEQ ID NO: 2258)
GGGguaggua,
(SEQ ID NO: 4507)
GAGguauugc,
(SEQ ID NO: 4508)
AUUguacaca,
(SEQ ID NO: 4509)
GAAguaggua,
(SEQ ID NO: 2212)
GGAguaagcu,
(SEQ ID NO: 2553)
UAGguaugug,
(SEQ ID NO: 2007)
GAGgugaaua,
(SEQ ID NO: 2056)
GAGgugggau,
(SEQ ID NO: 163)
AAGguaaucu,
(SEQ ID NO: 4510)
GGUgugaguu,
(SEQ ID NO: 4511)
AACgugaguu,
(SEQ ID NO: 4512)
GAGguaaccg,
(SEQ ID NO: 2488)
UAGguaagga,
(SEQ ID NO: 4513)
AUUguaagaa,
(SEQ ID NO: 2870)
UGGgugagca,
(SEQ ID NO: 150)
AAGguaaggc,
(SEQ ID NO: 4514)
CCAguaucgu,
(SEQ ID NO: 4515)
CCGgugggug,
(SEQ ID NO: 4516)
GAGguagugu,
(SEQ ID NO: 4517)
ACGgugggaa,
(SEQ ID NO: 2011)
GAGgugaccu,
(SEQ ID NO: 4518)
CACguaugua,
(SEQ ID NO: 799)
AGGgugggga,
(SEQ ID NO: 490)
AAUguaaguc,
(SEQ ID NO: 70)
AAAguuaagu,
(SEQ ID NO: 1541)
CAUgugagug,
(SEQ ID NO: 694)
AGAguauguc,
(SEQ ID NO: 4519)
GCGguaugac,
(SEQ ID NO: 1643)
CGGgugaguu,
(SEQ ID NO: 4520)
CCGguauuuu,
(SEQ ID NO: 4521)
GAGguagaac,
(SEQ ID NO: 2545)
UAGguaugaa,
(SEQ ID NO: 4522)
CAGgcgcgug,
(SEQ ID NO: 1027)
CAAguaaguc,
(SEQ ID NO: 816)
AGUguaagau,
(SEQ ID NO: 4523)
AAGguucuac,
(SEQ ID NO: 1555)
CCAguaagua,
(SEQ ID NO: 4524)
GAGguagcag,
(SEQ ID NO: 1312)
CAGgucuguu,
(SEQ ID NO: 1162)
CAGguacaau,
(SEQ ID NO: 1574)
CCGguaaaga,
(SEQ ID NO: 4525)
UAAgugcugu,
(SEQ ID NO: 786)
AGGgugagaa,
(SEQ ID NO: 4526)
CUCguaaggu,
(SEQ ID NO: 4527)
CAGgucagcu,
(SEQ ID NO: 1144)
CAGguaaggc,
(SEQ ID NO: 4528)
AGGgugcagg,
(SEQ ID NO: 4529)
GAGgugaaac,
(SEQ ID NO: 740)
AGGguaagua,
(SEQ ID NO: 4530)
AAUguaugcc,
(SEQ ID NO: 145)
AAGguaagca,
(SEQ ID NO: 587)
ACGguacggu,
(SEQ ID NO: 164)
AAGguaauga,
(SEQ ID NO: 4531)
UCUgcucaau,
(SEQ ID NO: 4532)
ACGguaaugu,
(SEQ ID NO: 4533)
AAGguaguug,
(SEQ ID NO: 580)
ACGguaagug,
(SEQ ID NO: 4534)
CAGgugauga,
(SEQ ID NO: 4535)
GAGguaacac,
(SEQ ID NO: 1937)
GAGguaggua,
(SEQ ID NO: 1179)
CAGguaccuu,
(SEQ ID NO: 1150)
CAGguaauaa,
(SEQ ID NO: 3016)
UUGgugggug,
(SEQ ID NO: 1710)
CUGguaauga,
(SEQ ID NO: 2492)
UAGguaaguc,
(SEQ ID NO: 4536)
AGGgugugac,
(SEQ ID NO: 4537)
GAGgcaauaa,
(SEQ ID NO: 4538)
GUGguaaagc,
(SEQ ID NO: 4539)
CUGgugggcg,
(SEQ ID NO: 2128)
GAUguauguu,
(SEQ ID NO: 787)
AGGgugagac,
(SEQ ID NO: 4540)
UCGgucagca,
(SEQ ID NO: 4541)
AUGgugauua,
(SEQ ID NO: 4542)
CGAgugugua,
(SEQ ID NO: 1488)
CAGguuggug,
(SEQ ID NO: 4543)
AGCgcaagua,
(SEQ ID NO: 4544)
UGGguacguu,
(SEQ ID NO: 1974)
GAGguauuug,
(SEQ ID NO: 4545)
AGUguacaua,
(SEQ ID NO: 898)
AUGguaagua,
(SEQ ID NO: 4546)
ACAguagguu,
(SEQ ID NO: 337)
AAGgugagag,
(SEQ ID NO: 4547)
UUGgugaagu,
(SEQ ID NO: 43)
AAAguaugua,
(SEQ ID NO: 4548)
UGGguaagga,
(SEQ ID NO: 4549)
UAGgugccuu,
and
(SEQ ID NO: 4550)
CCUgugggug.

Additional exemplary gene sequences and splice site sequences (e.g., 5′ splice site sequences) include

(SEQ ID NO: 4551)
UCCguaaguu,
(SEQ ID NO: 4552)
GUGguaaacg,
(SEQ ID NO: 4553)
CGGgugcggu,
(SEQ ID NO: 4554)
CAUguacuuc,
(SEQ ID NO: 4555)
AGAguaaagg,
(SEQ ID NO: 4556)
CGCgugagua,
(SEQ ID NO: 4557)
AGAgugggca,
(SEQ ID NO: 4558)
AGAguaagcc,
(SEQ ID NO: 4559)
AGAguaaaca,
(SEQ ID NO: 4560)
GUGguuauga,
(SEQ ID NO: 4561)
AGGguaauaa,
(SEQ ID NO: 4562)
UGAguaagac,
(SEQ ID NO: 4563)
AGAguuuguu,
(SEQ ID NO: 4564)
CGGgucugca,
(SEQ ID NO: 4565)
CAGguaaguc,
(SEQ ID NO: 4566)
AAGguagaau,
(SEQ ID NO: 4567)
CAGgucccuc,
(SEQ ID NO: 4568)
AGAguaaugg,
(SEQ ID NO: 4569)
GAGgucuaag,
(SEQ ID NO: 4570)
AGAguagagu,
(SEQ ID NO: 4571)
AUGgucagua,
(SEQ ID NO: 4572)
GAGgccuggg,
(SEQ ID NO: 4573)
AAGguguggc,
(SEQ ID NO: 4574)
AGAgugaucu,
(SEQ ID NO: 4575)
AAGguaucca,
(SEQ ID NO: 4576)
UUCguaagua,
(SEQ ID NO: 4577)
UAAgugggug,
(SEQ ID NO: 4578)
GCCgugaacg,
(SEQ ID NO: 4579)
GAGguugugg,
(SEQ ID NO: 4580)
UAUguaugca,
(SEQ ID NO: 4581)
UGUguaacaa,
(SEQ ID NO: 4582)
AGGguauuag,
(SEQ ID NO: 4583)
UGAguauauc,
(SEQ ID NO: 4584)
AGAguuugug,
(SEQ ID NO: 4585)
GAGgucgcug,
(SEQ ID NO: 4586)
GAGgucaucg,
(SEQ ID NO: 4587)
ACGguaaagc,
(SEQ ID NO: 4588)
UGAguacuug,
(SEQ ID NO: 4589)
CGAgucgccg,
(SEQ ID NO: 4590)
CUGguacguc,
(SEQ ID NO: 4591)
AGGguauugc,
(SEQ ID NO: 4592)
GAAgugaaug,
(SEQ ID NO: 4593)
CAGaugaguc,
(SEQ ID NO: 4594)
UGGguauugg,
(SEQ ID NO: 4595)
UGAguaaaga,
(SEQ ID NO: 4596)
GUGguuccug,
(SEQ ID NO: 4597)
UGAgcaagua,
(SEQ ID NO: 4598)
UAUguaagag,
(SEQ ID NO: 4599)
AAGgucuugc,
(SEQ ID NO: 4600)
AAAgcaugug,
(SEQ ID NO: 4601)
AGAguacagu,
(SEQ ID NO: 4602)
GUGguaaucc,
(SEQ ID NO: 4603)
CAGguagagg,
(SEQ ID NO: 4604)
AAGguacaac,
(SEQ ID NO: 4605)
UGGgcagcau,
(SEQ ID NO: 4606)
CCGgucauca,
(SEQ ID NO: 4607)
CCGguuugua,
(SEQ ID NO: 4608)
UGAguaaggg,
(SEQ ID NO: 4609)
GAAguaugua,
(SEQ ID NO: 4610)
GGGguagcuc,
(SEQ ID NO: 4611)
GCUguacaua,
(SEQ ID NO: 4612)
CUGgucucuu,
(SEQ ID NO: 4613)
GUGguaaaug,
(SEQ ID NO: 4614)
AUCguaagug,
(SEQ ID NO: 4615)
GAGgcaugua,
(SEQ ID NO: 4616)
AAGgucuccc,
(SEQ ID NO: 4617)
UGGgugcguu,
(SEQ ID NO: 4618)
UGUguagguu,
(SEQ ID NO: 4619)
GAAgugagca,
(SEQ ID NO: 4620)
GGUguaauuu,
(SEQ ID NO: 4621)
CUGgugaaau,
(SEQ ID NO: 4622)
AUCguaaguc,
(SEQ ID NO: 4623)
AGAguaaucc,
(SEQ ID NO: 4624)
GGAguagguc,
(SEQ ID NO: 4625)
GAGguaccaa,
(SEQ ID NO: 4626)
CUUguaggug,
(SEQ ID NO: 4627)
AAGguauaag,
(SEQ ID NO: 4628)
AGAguuggua,
(SEQ ID NO: 4629)
AUGguuugug,
(SEQ ID NO: 4630)
UGGgucagau,
(SEQ ID NO: 4631)
AGAguaggac,
(SEQ ID NO: 4632)
AGAguagugu,
(SEQ ID NO: 4633)
AGAguaggag,
(SEQ ID NO: 4634)
CAGgucucua,
(SEQ ID NO: 4635)
AAGguggaug,
(SEQ ID NO: 4636)
UGGguaucaa,
(SEQ ID NO: 4637)
GAUguaugga,
(SEQ ID NO: 4638)
AAGguguuuc,
(SEQ ID NO: 4639)
GCAguguaaa,
(SEQ ID NO: 4640)
UUAguaugua,
(SEQ ID NO: 4641)
UCUguaugca,
(SEQ ID NO: 4642)
AAUguaaaau,
(SEQ ID NO: 4643)
AGAguaaauu,
(SEQ ID NO: 4644)
GGGguacuuu,
(SEQ ID NO: 4645)
GAAguuugau,
(SEQ ID NO: 4646)
AAAguagauu,
(SEQ ID NO: 4647)
UGUguagagu,
(SEQ ID NO: 4648)
UGGguaagcg,
(SEQ ID NO: 4649)
CGGguucagg,
(SEQ ID NO: 4650)
AGGguacgac,
(SEQ ID NO: 4651)
UCGguaagaa,
(SEQ ID NO: 4652)
AGGguuggca,
(SEQ ID NO: 4653)
AAAguacagu,
(SEQ ID NO: 4654)
UAAguuaagg,
(SEQ ID NO: 4655)
AUGguaaugu,
(SEQ ID NO: 4656)
GUGguuuuac,
(SEQ ID NO: 4657)
AGAguaacaa,
(SEQ ID NO: 4658)
AAGguagccc,
(SEQ ID NO: 4659)
GCGgugaggc,
(SEQ ID NO: 4660)
AUGguucagc,
(SEQ ID NO: 4661)
AAGguacuua,
(SEQ ID NO: 4662)
AAGguccgug,
(SEQ ID NO: 4663)
UAGguaagcg,
(SEQ ID NO: 4664)
AUGguaccuu,
(SEQ ID NO: 4665)
GCCguggugg,
(SEQ ID NO: 4666)
CUGgugcguc,
(SEQ ID NO: 4667)
CAGguggaaa,
(SEQ ID NO: 4668)
AAAgucugua,
(SEQ ID NO: 4669)
GAGguaaccc,
(SEQ ID NO: 4670)
AGAguauggg,
(SEQ ID NO: 4671)
UAUgccccug,
(SEQ ID NO: 4672)
AAGgugccag,
(SEQ ID NO: 4673)
ACGgugcggc,
(SEQ ID NO: 4674)
AGGguacuga,
(SEQ ID NO: 4675)
AGAguaagcg,
(SEQ ID NO: 4676)
CUGgcaaggg,
(SEQ ID NO: 4677)
CCAgugugug,
(SEQ ID NO: 4678)
GAGguagacg,
(SEQ ID NO: 4679)
CGGgugcggg,
(SEQ ID NO: 4680)
GAUguaagcu,
(SEQ ID NO: 4681)
AUUguauuua,
(SEQ ID NO: 4682)
UGCgugagug,
(SEQ ID NO: 4683)
CUGgucuaua,
(SEQ ID NO: 4684)
GAGgugcuag,
(SEQ ID NO: 4685)
GAGgugccau,
(SEQ ID NO: 4686)
CAGguacguc,
(SEQ ID NO: 4687)
GAGguucagc,
(SEQ ID NO: 4688)
AACguaagaa,
(SEQ ID NO: 4689)
AGAguaguac,
(SEQ ID NO: 4690)
AAGguaacgg,
(SEQ ID NO: 4691)
UAGgugugac,
(SEQ ID NO: 4692)
CCGguaauag,
(SEQ ID NO: 4693)
CAGguaccag,
(SEQ ID NO: 4694)
UUUguaauug,
(SEQ ID NO: 4695)
AAUguacgaa,
(SEQ ID NO: 4696)
CAGguaauga,
(SEQ ID NO: 4697)
AUCgucaagg,
(SEQ ID NO: 4698)
CUGguagaug,
(SEQ ID NO: 4699)
GGGgugcagu,
(SEQ ID NO: 4700)
AGUgugagaa,
(SEQ ID NO: 4701)
GGGguuuuau,
(SEQ ID NO: 4702)
CCUguccccu,
(SEQ ID NO: 4703)
AUUgugaagu,
(SEQ ID NO: 4704)
AAGguaaacg,
(SEQ ID NO: 4705)
UACgucgugg,
(SEQ ID NO: 4706)
AAGgugccau,
(SEQ ID NO: 4707)
GGGgucccag,
(SEQ ID NO: 4708)
UAUguauggu,
(SEQ ID NO: 4709)
CGGguaauua,
(SEQ ID NO: 4710)
CGGguacucc,
(SEQ ID NO: 4711)
CAGgugacuu,
(SEQ ID NO: 4712)
AGUguggguu,
(SEQ ID NO: 4713)
AGAguauggc,
(SEQ ID NO: 4714)
AAGgccaaca,
(SEQ ID NO: 4715)
AAAgcaagua,
(SEQ ID NO: 4716)
UCAguagguc,
(SEQ ID NO: 4717)
GUGguggcgg,
(SEQ ID NO: 4718)
CAUguauccu,
(SEQ ID NO: 4719)
UCGgugagcc,
(SEQ ID NO: 4720)
AUAguugggu,
(SEQ ID NO: 4721)
AAUguuagcu,
(SEQ ID NO: 4722)
AUGgugaaug,
(SEQ ID NO: 4723)
CGGguaaugu,
(SEQ ID NO: 4724)
UCUguaggug,
(SEQ ID NO: 4725)
CCGgugaggc,
(SEQ ID NO: 4726)
UGAguccacu,
(SEQ ID NO: 4727)
CUAguaagag,
(SEQ ID NO: 4728)
CGGguggggc,
(SEQ ID NO: 4729)
CGAguaagca,
(SEQ ID NO: 4730)
UGUgccaauu,
(SEQ ID NO: 4731)
UCGguaagcc,
(SEQ ID NO: 4732)
UAUguaggug,
(SEQ ID NO: 4733)
UUGgugggcc,
(SEQ ID NO: 4734)
GAGgcugggc,
(SEQ ID NO: 4735)
AGAguaacuu,
(SEQ ID NO: 4736)
ACGguagguc,
(SEQ ID NO: 4737)
CAGgcccaga,
(SEQ ID NO: 4738)
CCGguggguu,
(SEQ ID NO: 4739)
AAGgugacgg,
(SEQ ID NO: 4740)
GGGguacagc,
(SEQ ID NO: 4741)
CAUguaaguc,
(SEQ ID NO: 4742)
AUUgugagaa,
(SEQ ID NO: 4743)
UGUguaagga,
(SEQ ID NO: 4744)
UUUguaagau,
(SEQ ID NO: 4745)
AGGgucauuu,
(SEQ ID NO: 4746)
UGGguuuguu,
(SEQ ID NO: 4747)
CGAguaagcc,
(SEQ ID NO: 4748)
GUGgugugua,
(SEQ ID NO: 4749)
AUGguauaac,
(SEQ ID NO: 4750)
UGGguacgua,
(SEQ ID NO: 4751)
AAAguagagu,
(SEQ ID NO: 4752)
UCGguaacug,
(SEQ ID NO: 4753)
AGAguaauga,
(SEQ ID NO: 4754)
AUGguggguc,
(SEQ ID NO: 4755)
AGAguaauau,
(SEQ ID NO: 4756)
CAGguacugg,
(SEQ ID NO: 4757)
UAAgucaguu,
(SEQ ID NO: 4758)
GCGguagaga,
(SEQ ID NO: 4759)
AAGgugaugg,
(SEQ ID NO: 4760)
ACAguauguu,
(SEQ ID NO: 4761)
GAUguacguc,
(SEQ ID NO: 4762)
UAGguuucuc,
(SEQ ID NO: 4763)
GAGgcauggg,
(SEQ ID NO: 4764)
AUAgcuaagu,
(SEQ ID NO: 4765)
GUAgucugua,
(SEQ ID NO: 4766)
AAGgugaacg,
(SEQ ID NO: 4767)
GUGguggucg,
(SEQ ID NO: 4768)
GAGguugauc,
(SEQ ID NO: 4769)
UGAguggguu,
(SEQ ID NO: 4770)
ACUguacgug,
(SEQ ID NO: 4771)
CUGgugacug,
(SEQ ID NO: 4772)
CAAguuaagc,
(SEQ ID NO: 4773)
GAGguaccca,
(SEQ ID NO: 4774)
AACguaacuu,
(SEQ ID NO: 4775)
CAGguuacua,
(SEQ ID NO: 4776)
AGAguuaguc,
(SEQ ID NO: 4777)
UGGgcacguc,
(SEQ ID NO: 4778)
AGUguauggu,
(SEQ ID NO: 4779)
AAGguugcaa,
(SEQ ID NO: 4780)
CAGguuguua,
(SEQ ID NO: 4781)
AAGgcauccc,
(SEQ ID NO: 4782)
GAUguaaggc,
(SEQ ID NO: 4783)
AGGguacggg,
(SEQ ID NO: 4784)
GAGgucaaag,
(SEQ ID NO: 4785)
CAAgugagcg,
(SEQ ID NO: 4786)
AGAguaaucu,
(SEQ ID NO: 4787)
UCGguagcug,
(SEQ ID NO: 4788)
AAAguaguag,
(SEQ ID NO: 4789)
CAGguucguc,
(SEQ ID NO: 4790)
CGUguaugaa,
(SEQ ID NO: 4791)
AGUguaaaaa,
(SEQ ID NO: 4792)
AAGgucucac,
(SEQ ID NO: 4793)
UAGguggagc,
(SEQ ID NO: 4794)
UGAguaggug,
(SEQ ID NO: 4795)
AGAguaugcc,
(SEQ ID NO: 4796)
GAGguugcau,
(SEQ ID NO: 4797)
CAAguaagag,
(SEQ ID NO: 4798)
UCUgugugcc,
(SEQ ID NO: 4799)
GAGgugaugc,
(SEQ ID NO: 4800)
GGGgugauaa,
(SEQ ID NO: 4801)
CCCgugagcc,
(SEQ ID NO: 4802)
AGAguaacug,
(SEQ ID NO: 4803)
GCGguaagua,
(SEQ ID NO: 4804)
AGAguacauc,
(SEQ ID NO: 4805)
UCGgucuggg,
(SEQ ID NO: 4806)
UAAguaucuc,
(SEQ ID NO: 4807)
GGCguagguu,
(SEQ ID NO: 4808)
AGAguacgcc,
(SEQ ID NO: 4809)
GAUgucuucu,
(SEQ ID NO: 4810)
AGGgcaaggu,
(SEQ ID NO: 4811)
CGAguaugau,
(SEQ ID NO: 4812)
AUGguagagu,
(SEQ ID NO: 4813)
CAAguacgag,
(SEQ ID NO: 4814)
UCGguaugau,
(SEQ ID NO: 4815)
CCGguguguu,
(SEQ ID NO: 4816)
AGGgucugug,
(SEQ ID NO: 4817)
GGAguaggcu,
(SEQ ID NO: 4818)
AAGgucuaug,
(SEQ ID NO: 4819)
GCAgugcgug,
(SEQ ID NO: 4820)
UGGgugagaa,
(SEQ ID NO: 4821)
AGGguaaagu,
(SEQ ID NO: 4822)
GAGguaggac,
(SEQ ID NO: 4823)
CUAguaagca,
(SEQ ID NO: 4824)
UUAguaggcu,
(SEQ ID NO: 4825)
CUGgugggau,
(SEQ ID NO: 4826)
CUGguuagua,
(SEQ ID NO: 4827)
AAGguacgug,
(SEQ ID NO: 4828)
CGGgugagau,
(SEQ ID NO: 4829)
AAGgugcaug,
(SEQ ID NO: 4830)
AAUgugggcu,
(SEQ ID NO: 4831)
CAGguugacu,
(SEQ ID NO: 4832)
CAGguuacag,
(SEQ ID NO: 4833)
GCGguaacau,
(SEQ ID NO: 4834)
AUUgucaguc,
(SEQ ID NO: 4835)
CAAguauaca,
(SEQ ID NO: 4836)
GAUguccgcc,
(SEQ ID NO: 4837)
AAGgugcgga,
(SEQ ID NO: 4838)
AACguaagag,
(SEQ ID NO: 4839)
UGGguuggua,
(SEQ ID NO: 4840)
CAAguguaag,
(SEQ ID NO: 4841)
GUGguaacgu,
(SEQ ID NO: 4842)
CUGgugauca,
(SEQ ID NO: 4843)
AGGguggggc,
(SEQ ID NO: 4844)
UCGguaaaga,
(SEQ ID NO: 4845)
CAGguacacc,
(SEQ ID NO: 4846)
CGGguaaggg,
(SEQ ID NO: 4847)
CAAguuugcu,
(SEQ ID NO: 4848)
ACAgugcgug,
(SEQ ID NO: 4849)
UUGguauggg,
(SEQ ID NO: 4850)
GAGgcucauc,
(SEQ ID NO: 4851)
CUGguaauag,
(SEQ ID NO: 4852)
AUGguggaua,
(SEQ ID NO: 4853)
UCAgugaauu,
(SEQ ID NO: 4854)
AAUguaauua,
(SEQ ID NO: 4855)
GCAgucuaaa,
(SEQ ID NO: 4856)
AAGguauucu,
(SEQ ID NO: 4857)
GAGgucauca,
(SEQ ID NO: 4858)
UGGguccaug,
(SEQ ID NO: 4859)
AGAguuugua,
(SEQ ID NO: 4860)
AGGguagacu,
(SEQ ID NO: 4861)
AAGguaggac,
(SEQ ID NO: 4862)
UGUguguuga,
(SEQ ID NO: 4863)
UCAguacgug,
(SEQ ID NO: 4864)
AUGgucucuc,
(SEQ ID NO: 4865)
UGAguuagua,
(SEQ ID NO: 4866)
UGAguaaagu,
(SEQ ID NO: 4867)
GAGgugaccg,
(SEQ ID NO: 4868)
GAGguauauc,
(SEQ ID NO: 4869)
CAGgugccau,
(SEQ ID NO: 4870)
AGAgugguga,
(SEQ ID NO: 4871)
GUUguaagaa,
(SEQ ID NO: 4872)
AGAguaaaua,
(SEQ ID NO: 4873)
AGGgugaagg,
(SEQ ID NO: 4874)
CUGguagauu,
(SEQ ID NO: 4875)
GAGguucagg,
(SEQ ID NO: 4876)
AGGgucuuca,
(SEQ ID NO: 4877)
CUGguaaccu,
(SEQ ID NO: 4878)
ACAguacuga,
(SEQ ID NO: 4879)
AGAguggguc,
(SEQ ID NO: 4880)
AUGguaugag,
(SEQ ID NO: 4881)
AAGguuauau,
(SEQ ID NO: 4882)
AGAguauagu,
(SEQ ID NO: 4883)
AAAguaugaa,
(SEQ ID NO: 4884)
UAGguggcua,
(SEQ ID NO: 4885)
ACCguauggg,
(SEQ ID NO: 4886)
AAAguauaau,
(SEQ ID NO: 4887)
UUUguauggc,
(SEQ ID NO: 4888)
GGGgucgcgu,
(SEQ ID NO: 4889)
GUGgugguuu,
(SEQ ID NO: 4890)
CAGguuugac,
(SEQ ID NO: 4891)
GGAguaggcg,
(SEQ ID NO: 4892)
GAGguacccu,
(SEQ ID NO: 4893)
AUGgugugca,
(SEQ ID NO: 4894)
GUGguuggug,
(SEQ ID NO: 4895)
AAAguaugcu,
(SEQ ID NO: 4896)
UAAguuacau,
(SEQ ID NO: 4897)
ACAguaugag,
(SEQ ID NO: 4898)
GGAguauguu,
(SEQ ID NO: 4899)
UUUgugagaa,
(SEQ ID NO: 4900)
AAUgugcguu,
(SEQ ID NO: 4901)
CAGguagagu,
(SEQ ID NO: 4902)
AUGguguuaa,
(SEQ ID NO: 4903)
CAUgugcguc,
(SEQ ID NO: 4904)
AUAguuggau,
(SEQ ID NO: 4905)
GAGguacgua ,
(SEQ ID NO: 4906)
GUUgugagaa,
(SEQ ID NO: 4907)
CAAguacauc,
(SEQ ID NO: 4908)
GAGguaguuu,
(SEQ ID NO: 4909)
ACUguacaga,
(SEQ ID NO: 4910)
CCGguuguga,
(SEQ ID NO: 4911)
UGGgucagug,
(SEQ ID NO: 4912)
GUAguaagaa,
(SEQ ID NO: 4913)
GACguacuuu,
(SEQ ID NO: 4914)
AGAgucaguc,
(SEQ ID NO: 4915)
UAGguuaguu,
(SEQ ID NO: 4916)
AGGgcagcag,
(SEQ ID NO: 4917)
AAGguccuac,
(SEQ ID NO: 4918)
AAUguaauug,
(SEQ ID NO: 4919)
CAGgugcggg,
(SEQ ID NO: 4920)
CUGguaaugg,
(SEQ ID NO: 4921)
CAAguagccc,
(SEQ ID NO: 4922)
GAAgucaguu,
(SEQ ID NO: 4923)
ACAguaauug,
(SEQ ID NO: 4924)
UUAguuagua,
(SEQ ID NO: 4925)
CCUguauuuu,
(SEQ ID NO: 4926)
AUCguaagaa,
(SEQ ID NO: 4927)
CCAgugagca,
(SEQ ID NO: 4928)
GAAguaaggc,
(SEQ ID NO: 4929)
UGAgugggua,
(SEQ ID NO: 4930)
UCAgugguag,
(SEQ ID NO: 4931)
UCUguacagg,
(SEQ ID NO: 4932)
CGAgugagug,
(SEQ ID NO: 4933)
UCCguaugug,
(SEQ ID NO: 4934)
CAUgccguuu,
(SEQ ID NO: 4935)
AAAgugacuu,
(SEQ ID NO: 4936)
AGAguaggca,
(SEQ ID NO: 4937)
GAAguaagag,
(SEQ ID NO: 4938)
CAGgcagguu,
(SEQ ID NO: 4939)
UUGguagagc,
(SEQ ID NO: 4940)
AAGguggaaa,
(SEQ ID NO: 4941)
GAGgcagguc,
(SEQ ID NO: 4942)
AUGguacgac,
(SEQ ID NO: 4943)
AGGguaggaa,
(SEQ ID NO: 4944)
AGGguaggua,
(SEQ ID NO: 4945)
UUGguaaggu,
(SEQ ID NO: 4946)
AUGguacaga,
(SEQ ID NO: 4947)
CAGguagagc,
(SEQ ID NO: 4948)
UAGguaaggu,
(SEQ ID NO: 4949)
GGGguuagag,
(SEQ ID NO: 4950)
AAGguaucaa,
(SEQ ID NO: 4951)
GAGguagccc,
(SEQ ID NO: 4952)
CAGgugccuc,
(SEQ ID NO: 4953)
GCAguaagag,
(SEQ ID NO: 4954)
ACGguagagu,
(SEQ ID NO: 4955)
UGGguaaugg,
(SEQ ID NO: 4956)
CUGgucaguu,
(SEQ ID NO: 4957)
GUGguacauu,
(SEQ ID NO: 4958)
AAAguagguu,
(SEQ ID NO: 4959)
AAGgccaaga,
(SEQ ID NO: 4960)
CGGgugggca,
(SEQ ID NO: 4961)
ACGguccggg,
(SEQ ID NO: 4962)
CGAguaugag,
(SEQ ID NO: 4963)
CUGguaugcc,
(SEQ ID NO: 4964)
GAGguggaug,
(SEQ ID NO: 4965)
CAGgccuuuc,
(SEQ ID NO: 4966)
AAAguacauc,
(SEQ ID NO: 4967)
AAAguaauca,
(SEQ ID NO: 4968)
GAGguaacug,
(SEQ ID NO: 4969)
UGguaaaga,
(SEQ ID NO: 4970)
CGUguaagca,
(SEQ ID NO: 4971)
UGGgcaagua,
(SEQ ID NO: 4972)
GCGguggcga,
(SEQ ID NO: 4973)
GAGguggccg,
(SEQ ID NO: 4974)
AUUgcaugca,
(SEQ ID NO: 4975)
ACGgugacug,
(SEQ ID NO: 4976)
CAGgucagau,
(SEQ ID NO: 4977)
AGAguaacuc,
(SEQ ID NO: 4978)
UGAguaacag,
(SEQ ID NO: 4979)
AAGguacccg,
(SEQ ID NO: 4980)
AGGguaggcu,
(SEQ ID NO: 4981)
GGGgcaggac,
(SEQ ID NO: 4982)
CCUguaagug,
(SEQ ID NO: 4983)
AUUguaagug,
(SEQ ID NO: 4984)
ACUguacgag,
(SEQ ID NO: 4985)
GUAguagugu,
(SEQ ID NO: 4986)
AGAguaugag,
(SEQ ID NO: 4987)
UCAguguggg,
(SEQ ID NO: 4988)
UGGguauaua,
(SEQ ID NO: 4989)
UAGguagcua,
(SEQ ID NO: 4990)
GGGguaaaga,
(SEQ ID NO: 4991)
AGGguuacuu,
(SEQ ID NO: 4992)
CAUguaaaug,
(SEQ ID NO: 4993)
GGAguaguaa,
(SEQ ID NO: 4994)
CAGgucaauc,
(SEQ ID NO: 4995)
CGGguuagug,
(SEQ ID NO: 4996)
UAGguacaug,
(SEQ ID NO: 4997)
UAGguuaaga,
(SEQ ID NO: 4998)
UGGguaccuu,
(SEQ ID NO: 4999)
CGGguggaca,
(SEQ ID NO: 5000)
CAGgucuuac,
(SEQ ID NO: 5001)
AAGguggagc,
(SEQ ID NO: 5002)
AUGguaacca,
(SEQ ID NO: 5003)
UCGguaaguu,
(SEQ ID NO: 5004)
UAUguacaaa,
(SEQ ID NO: 5005)
AAUguagauu,
(SEQ ID NO: 5006)
GUAgcuagua,
(SEQ ID NO: 5007)
AAGguauugg,
(SEQ ID NO: 5008)
GAGgucuuug,
(SEQ ID NO: 5009)
GAAguucagg,
(SEQ ID NO: 5010)
UGGguaucac,
(SEQ ID NO: 5011)
AGAguacugg,
(SEQ ID NO: 5012)
CAGguuaaug,
(SEQ ID NO: 5013)
AGGguacgug,
(SEQ ID NO: 5014)
AGGgcacagg,
(SEQ ID NO: 5015)
CUGguuaguu,
(SEQ ID NO: 5016)
UUGguacgag,
(SEQ ID NO: 5017)
ACGgugauca,
(SEQ ID NO: 5018)
CCUgugagag,
(SEQ ID NO: 5019)
GAGgugaagu,
(SEQ ID NO: 5020)
AAGguacauc,
(SEQ ID NO: 5021)
UCUguaugug,
(SEQ ID NO: 5022)
UUGguggaag,
(SEQ ID NO: 5023)
UGGgcagguu,
(SEQ ID NO: 5024)
GAAguggagc,
(SEQ ID NO: 5025)
ACAguaagac,
(SEQ ID NO: 5026)
CGGguaccaa,
(SEQ ID NO: 5027)
CAAguacguc,
(SEQ ID NO: 5028)
AGAgugaggg,
(SEQ ID NO: 5029)
CGGguaagaa,
(SEQ ID NO: 5030)
AAUguaggug,
(SEQ ID NO: 5031)
AUCgugugcu,
(SEQ ID NO: 5032)
UAGgucaugg,
(SEQ ID NO: 5033)
CAGguuuuga,
(SEQ ID NO: 5034)
AAGgcaugca,
(SEQ ID NO: 5035)
GAGgugcugc,
(SEQ ID NO: 5036)
AAGguuaaua,
(SEQ ID NO: 5037)
CAGguucauc,
(SEQ ID NO: 5038)
GCGguaggug,
(SEQ ID NO: 5039)
GACgugagua,
(SEQ ID NO: 5040)
CAGgucuacu,
(SEQ ID NO: 5041)
UUGguaugag,
(SEQ ID NO: 5042)
AGCgugggca,
(SEQ ID NO: 5043)
AUGguaaggu,
(SEQ ID NO: 5044)
AUGguaccuc,
(SEQ ID NO: 5045)
UUGguauggu,
(SEQ ID NO: 5046)
UAUguaugaa,
(SEQ ID NO: 5047)
UGGguauggg,
(SEQ ID NO: 5048)
GAUguaaaua,
(SEQ ID NO: 5049)
CCGguaaguu,
(SEQ ID NO: 5050)
GAGgucugaa,
(SEQ ID NO: 5051)
GAGgugcgag,
(SEQ ID NO: 5052)
CUGgucagcc,
(SEQ ID NO: 5053)
CAGguuuugu,
(SEQ ID NO: 5054)
CGGguggugu,
(SEQ ID NO: 5055)
UAAguuagua,
(SEQ ID NO: 5056)
UUUgugugug,
(SEQ ID NO: 5057)
CAGguuaacc,
(SEQ ID NO: 5058)
UUGguacuuu,
(SEQ ID NO: 5059)
GCUguaaggc,
(SEQ ID NO: 5060)
AGGguggcug,
(SEQ ID NO: 5061)
GAUguaaaaa,
(SEQ ID NO: 5062)
AAGgucaaaa,
(SEQ ID NO: 5063)
CAGguagcgc,
(SEQ ID NO: 5064)
CAGguuuggc,
(SEQ ID NO: 5065)
GAGgugguuu,
(SEQ ID NO: 5066)
CGGguaaaua,
(SEQ ID NO: 5067)
CUGguucggu,
(SEQ ID NO: 5068)
GGAgugagcc,
(SEQ ID NO: 5069)
AAGgugcgcg,
(SEQ ID NO: 5070)
GAAguacauc,
(SEQ ID NO: 5071)
AGUgucugua,
(SEQ ID NO: 5072)
CCCgugagcu,
(SEQ ID NO: 5073)
GAGguucaca,
(SEQ ID NO: 5074)
CUAgugggua,
(SEQ ID NO: 5075)
GAGguaacua,
(SEQ ID NO: 5076)
UCGguauguc,
(SEQ ID NO: 5077)
UAAguauuug,
(SEQ ID NO: 5078)
CAGguaagcg,
(SEQ ID NO: 5079)
GAGgugguaa,
(SEQ ID NO: 5080)
CGAguaagag,
(SEQ ID NO: 5081)
CCGguaagcu,
(SEQ ID NO: 5082)
GAGgucuugu,
(SEQ ID NO: 5083)
AAGguggguc,
(SEQ ID NO: 5084)
CACguaagug,
(SEQ ID NO: 5085)
AGUguaauga,
(SEQ ID NO: 5086)
AAAgugugua,
(SEQ ID NO: 5087)
GGAgugccaa,
(SEQ ID NO: 5088)
CACgugaguu,
(SEQ ID NO: 5089)
AAGguuggau,
(SEQ ID NO: 5090)
UAUguaaaua,
(SEQ ID NO: 5091)
CUGguaggaa,
(SEQ ID NO: 5092)
UAUguaaacu,
(SEQ ID NO: 5093)
AAUguauuuu,
(SEQ ID NO: 5094)
CUGgcaagug,
(SEQ ID NO: 5095)
UGUgugguau,
(SEQ ID NO: 5096)
UAUguauguu,
(SEQ ID NO: 5097)
UUGgugacuc,
(SEQ ID NO: 5098)
GGAguaaggu,
(SEQ ID NO: 5099)
AAGguagaug,
(SEQ ID NO: 5100)
UGGguagggu,
(SEQ ID NO: 5101)
AAUguaauuc,
(SEQ ID NO: 5102)
GUGguauggc,
(SEQ ID NO: 5103)
GGAguggguu,
(SEQ ID NO: 5104)
AGGguaccac,
(SEQ ID NO: 5105)
UAGgugacag,
(SEQ ID NO: 5106)
ACAguaggca,
(SEQ ID NO: 5107)
AUGguuugaa,
(SEQ ID NO: 5108)
GCAguaacua,
(SEQ ID NO: 5109)
CCGguaggua,
(SEQ ID NO: 5110)
AGAguaggcc,
(SEQ ID NO: 5111)
AAGguugaca,
(SEQ ID NO: 5112)
CUGgugugua,
(SEQ ID NO: 5113)
GAAgucuguc,
(SEQ ID NO: 5114)
UGGgcucgga,
(SEQ ID NO: 5115)
CAGguagccu,
(SEQ ID NO: 5116)
AGAguaggua,
(SEQ ID NO: 5117)
UAAguauguc,
(SEQ ID NO: 5118)
CUGguauauc,
(SEQ ID NO: 5119)
GAGguguguu,
(SEQ ID NO: 5120)
AUGgugcaug,
(SEQ ID NO: 5121)
AAGguacgcc,
(SEQ ID NO: 5122)
UGAguaacua,
(SEQ ID NO: 5123)
GAGgugacag,
(SEQ ID NO: 5124)
GUUguccugu,
(SEQ ID NO: 5125)
UUGgugucuu,
(SEQ ID NO: 5126)
AAUgugaagg,
(SEQ ID NO: 5127)
UUGguggaua,
(SEQ ID NO: 5128)
UAGguguguu,
(SEQ ID NO: 5129)
CUGgcaaguu,
(SEQ ID NO: 5130)
GCAguaagau,
(SEQ ID NO: 5131)
GCGguggaaa,
(SEQ ID NO: 5132)
UGCguccagc,
(SEQ ID NO: 5133)
AAAguggagu,
(SEQ ID NO: 5134)
CGUgugagcc,
(SEQ ID NO: 5135
AGAguacugu),
(SEQ ID NO: 5136)
CAGguauagc,
(SEQ ID NO: 5137)
UACguaagga,
(SEQ ID NO: 5138)
AAGgucuuua,
(SEQ ID NO: 5139)
AAGguggucu,
(SEQ ID NO: 5140)
GGGguaaauu,
(SEQ ID NO: 5141)
UCAgugagga,
(SEQ ID NO: 5142)
AGAguacguu,
(SEQ ID NO: 5143)
GAGgucguca,
(SEQ ID NO: 5144)
UAGguuugau,
(SEQ ID NO: 5145)
CAUguaaacc,
(SEQ ID NO: 5146)
AAGguggcac,
(SEQ ID NO: 5147)
CAGguagaug,
(SEQ ID NO: 5148)
AACguaaaag,
(SEQ ID NO: 5149)
UAGgucucug,
(SEQ ID NO: 5150)
AUAguaggug,
(SEQ ID NO: 5151)
UAGgcaagag,
(SEQ ID NO: 5152)
UAGgcacggc,
(SEQ ID NO: 5153)
AAGgucuuca,
(SEQ ID NO: 5154)
CCAguaugcu,
(SEQ ID NO: 5155)
CAAgugaguu,
(SEQ ID NO: 5156)
CAGgucucaa,
(SEQ ID NO: 5157)
CAGguuacau,
(SEQ ID NO: 5158)
GGAgugagca,
(SEQ ID NO: 5159)
AGAguacgca,
(SEQ ID NO: 5160)
CUGguguugg,
(SEQ ID NO: 5161)
AAGguacuca,
(SEQ ID NO: 5162)
CUAguaaggg,
(SEQ ID NO: 5163)
AGAguaaaag,
(SEQ ID NO: 5164)
AAGguaacga,
(SEQ ID NO: 5165)
CUGguccccg,
(SEQ ID NO: 5166)
UAAguauggg,
(SEQ ID NO: 5167)
GAGgucgagc,
(SEQ ID NO: 5168)
UUGguauaua,
(SEQ ID NO: 5169)
AAAgucaagg,
(SEQ ID NO: 5170)
AAGgucuagg,
(SEQ ID NO: 5171)
CGAguagguc,
(SEQ ID NO: 5172)
AGGguucguu,
(SEQ ID NO: 5173)
GAGgcaggcc,
(SEQ ID NO: 5174)
CUAguauuac,
(SEQ ID NO: 5175)
ACGguaugug,
(SEQ ID NO: 5176)
UAGgugguuc,
(SEQ ID NO: 5177)
AGAguauaac,
(SEQ ID NO: 5178)
UUGgugcguc,
(SEQ ID NO: 5179)
ACCguuaucu,
(SEQ ID NO: 5180)
CCAgugauga,
(SEQ ID NO: 5181)
GAAguaugca,
(SEQ ID NO: 5182)
GAAguauggc,
(SEQ ID NO: 5183)
CCGguaggac,
(SEQ ID NO: 5184)
AAUguaagca,
(SEQ ID NO: 5185)
AGAguaauug,
(SEQ ID NO: 5186)
AGGguugguu,
(SEQ ID NO: 5187)
GUGguaggag,
(SEQ ID NO: 5188)
AAGgcaguuu,
(SEQ ID NO: 5189)
CAAguaagcc,
(SEQ ID NO: 5190)
CUGgcaagua,
(SEQ ID NO: 5191)
CAGgcaugau,
(SEQ ID NO: 5192)
AGGguaauug,
(SEQ ID NO: 5193)
GGGguaaccu,
(SEQ ID NO: 5194)
AAAguaacua,
(SEQ ID NO: 5195)
UAGgucugcc,
(SEQ ID NO: 5196)
ACGguaugaa,
(SEQ ID NO: 5197)
AGUguauggg,
(SEQ ID NO: 5198)
UGGguuggca,
(SEQ ID NO: 5199)
UAGguaaacu,
(SEQ ID NO: 5200)
AGAgugggua,
(SEQ ID NO: 5201)
AGAguauuug,
(SEQ ID NO: 5202)
AGUguaggaa,
(SEQ ID NO: 5203)
CUUguacgua,
(SEQ ID NO: 5204)
GAUgugagau,
(SEQ ID NO: 5205)
CAGgcagcca,
(SEQ ID NO: 5206)
AAGgucacug,
(SEQ ID NO: 5207)
AAGgucugac,
(SEQ ID NO: 5208)
UAGguuccuu,
(SEQ ID NO: 5209)
CUGgugcuuu,
(SEQ ID NO: 5210)
UGAguuggug,
(SEQ ID NO: 5211)
UUGgugggau,
(SEQ ID NO: 5212)
UGAguagggu,
(SEQ ID NO: 5213)
UCGgugaggu,
(SEQ ID NO: 5214)
AAAguaaaga,
(SEQ ID NO: 5215)
AAGgcaaguc,
(SEQ ID NO: 5216)
CGGguaaagc,
(SEQ ID NO: 5217)
AAAguuaguu,
(SEQ ID NO: 5218)
UUAguaagca,
(SEQ ID NO: 5219)
GAGgucacau,
(SEQ ID NO: 5220)
UAAgugguau,
(SEQ ID NO: 5221)
UAGgugcuuu,
(SEQ ID NO: 5222)
GGAguaggca,
(SEQ ID NO: 5223)
UGAguaagga,
(SEQ ID NO: 5224)
CAGguggagc,
(SEQ ID NO: 5225)
GAUguagaag,
(SEQ ID NO: 5226)
AAUgccugcc,
(SEQ ID NO: 5227)
AUGguaaggc,
(SEQ ID NO: 5228)
UGGguaauau,
(SEQ ID NO: 5229)
CUGguaccuc,
(SEQ ID NO: 5230)
CACgugagcc,
(SEQ ID NO: 5231)
UGAguuugug,
(SEQ ID NO: 5232)
CCGguagugu,
(SEQ ID NO: 5233)
AAAgugacaa,
(SEQ ID NO: 5234)
GAAguggguu,
(SEQ ID NO: 5235)
CAGgugcagc,
(SEQ ID NO: 5236)
GAGgugggcc,
(SEQ ID NO: 5237)
UAUgugcguc,
(SEQ ID NO: 5238)
GGGguacugg,
(SEQ ID NO: 5239)
CUGguagguu,
(SEQ ID NO: 5240)
UUGgcauguu,
(SEQ ID NO: 5241)
AAUguaauac,
(SEQ ID NO: 5242)
UAGgccggug,
(SEQ ID NO: 5243)
AGAgucagua,
(SEQ ID NO: 5244)
UAAguaaauc,
(SEQ ID NO: 5245)
CAGguuccuc,
(SEQ ID NO: 5246)
UAGguacgau,
(SEQ ID NO: 5247)
AGAguuagug,
(SEQ ID NO: 5248)
GCAguaagug,
(SEQ ID NO: 5249)
AGGgugguag,
(SEQ ID NO: 5250)
GGAguaaugu,
(SEQ ID NO: 5251)
GAUguaaguc,
(SEQ ID NO: 5252)
CCAguuucgu,
(SEQ ID NO: 5253)
AAGguucggg,
(SEQ ID NO: 5254)
AUGguggagu,
(SEQ ID NO: 5255)
AAGguaccgg,
(SEQ ID NO: 5256)
GAAgugcgaa,
(SEQ ID NO: 5257)
UGGgucaguu,
(SEQ ID NO: 5258)
AAGguguaga,
(SEQ ID NO: 5259)
UGGguaggcc,
(SEQ ID NO: 5260)
CCAgugaguc,
(SEQ ID NO: 5261)
AAGgucacuu,
(SEQ ID NO: 5262)
AGCgugaggc,
(SEQ ID NO: 5263)
UCCgugguaa,
(SEQ ID NO: 5264)
AGAguacuua,
(SEQ ID NO: 5265)
GGGgucagau,
(SEQ ID NO: 5266)
AAGguggacc,
(SEQ ID NO: 5267)
AGAgugagcg,
(SEQ ID NO: 5268)
AGAgucagau,
(SEQ ID NO: 5269)
UAAguauuac,
(SEQ ID NO: 5270)
AGAguauuuc,
(SEQ ID NO: 5271)
AGAguucagc,
(SEQ ID NO: 5272)
AUGgugaagu,
(SEQ ID NO: 5273)
UAGgugaucc,
(SEQ ID NO: 5274)
GGAguaagau,
(SEQ ID NO: 5275)
UAGguaccaa,
(SEQ ID NO: 5276)
AGAguugguc,
(SEQ ID NO: 5277)
GAAgugagac,
(SEQ ID NO: 5278)
AUCguagguu,
(SEQ ID NO: 5279)
GAGguacgcu,
(SEQ ID NO: 5280)
ACGguaaggg,
(SEQ ID NO: 5281)
CAGgcauguc,
(SEQ ID NO: 5282)
UUAguaagau,
(SEQ ID NO: 5283)
UGAguagguu,
(SEQ ID NO: 5284)
AGGguacgaa,
(SEQ ID NO: 5285)
ACGguauguu,
(SEQ ID NO: 5286)
AGGguacugu,
(SEQ ID NO: 5287)
UUGguaugga,
(SEQ ID NO: 5288)
UAAguaacug,
(SEQ ID NO: 5289)
GCGgucagcc,
(SEQ ID NO: 5290)
UUUgugaguc,
(SEQ ID NO: 5291)
GUGgucagug,
(SEQ ID NO: 5292)
CUGgucugua,
(SEQ ID NO: 5293)
GAGguucuua,
(SEQ ID NO: 5294)
AUGguacuga,
(SEQ ID NO: 5295)
AAUgugcuuu,
(SEQ ID NO: 5296)
AGGguggcgu,
(SEQ ID NO: 5297)
CCGgcaggaa,
(SEQ ID NO: 5298)
CAUguggguc,
(SEQ ID NO: 5299)
UUGguuuguu,
(SEQ ID NO: 5300)
CAGguucugu,
(SEQ ID NO: 5301)
ACGguaagcg,
(SEQ ID NO: 5302)
CUGgucagua,
(SEQ ID NO: 5303)
UCAguaggcu,
(SEQ ID NO: 5304)
UGAguaggac,
(SEQ ID NO: 5305)
CAGguuuuaa,
(SEQ ID NO: 5306)
GAGguguccc,
(SEQ ID NO: 5307)
AGGguggguu,
(SEQ ID NO: 5308)
GUGgugagac,
(SEQ ID NO: 5309)
CACguaggga,
(SEQ ID NO: 5310)
GUGguauuuu,
(SEQ ID NO: 5311)
GAGauauccu,
(SEQ ID NO: 5312)
AAGgugaaca,
(SEQ ID NO: 5313)
UAAguagggc,
(SEQ ID NO: 5314)
CUGgugcggg,
(SEQ ID NO: 5315)
CUGgucaaua,
(SEQ ID NO: 5316)
AGAguaaaaa,
(SEQ ID NO: 5317)
AAGgugcagu,
(SEQ ID NO: 5318)
CGGguaagca,
(SEQ ID NO: 5319)
AAAgugagcc,
(SEQ ID NO: 5320)
AUGguaauca,
(SEQ ID NO: 5321)
GCAguacgug,
(SEQ ID NO: 5322)
AUGguacaug,
(SEQ ID NO: 5323)
AAGguuaaga,
(SEQ ID NO: 5324)
CGGguaaaug,
(SEQ ID NO: 5325)
GAGguucgca,
(SEQ ID NO: 5326)
GAGgcucugg,
(SEQ ID NO: 5327)
AUGgugggac,
(SEQ ID NO: 5328)
AACgugguag,
(SEQ ID NO: 5329)
AAGgugauag,
(SEQ ID NO: 5330)
GGGguuugca,
(SEQ ID NO: 5331)
CAUguaaggg,
(SEQ ID NO: 5332)
UCAguugagu,
(SEQ ID NO: 5333)
AAAgugcggc,
(SEQ ID NO: 5334)
AGAgugagcc,
(SEQ ID NO: 5335)
AUGgcaagaa,
(SEQ ID NO: 5336)
ACAguaaggu,
(SEQ ID NO: 5337)
AAGgucucua,
(SEQ ID NO: 5338)
GUGguaaaaa,
(SEQ ID NO: 5339)
AAAguaggug,
(SEQ ID NO: 5340)
UAGgugcacu,
(SEQ ID NO: 5341)
GUCgugguau,
(SEQ ID NO: 5342)
CAGguauagg,
(SEQ ID NO: 5343)
UGAgugagag,
(SEQ ID NO: 5344)
ACUgugagcc,
(SEQ ID NO: 5345)
AUCguuaguu,
(SEQ ID NO: 5346)
UUUguaccaa,
(SEQ ID NO: 5347)
UGGgugagau,
(SEQ ID NO: 5348)
AGAgugagaa,
(SEQ ID NO: 5349)
AGAguagggg,
(SEQ ID NO: 5350)
AGGgcaagua,
(SEQ ID NO: 5351)
CGGgucagua,
(SEQ ID NO: 5352)
UUGguaugcc,
(SEQ ID NO: 5353)
CGGguuagau,
(SEQ ID NO: 5354)
GGGgugaagu,
(SEQ ID NO: 5355)
CCCgugugaa,
(SEQ ID NO: 5356)
GCAguuugga,
(SEQ ID NO: 5357)
UGCguaagac,
(SEQ ID NO: 5358)
AGAgucugua,
(SEQ ID NO: 5359)
CACgugagca,
(SEQ ID NO: 5360)
AGGguaaaag,
(SEQ ID NO: 5361)
CAGgcugggu,
(SEQ ID NO: 5362)
GAAgucuuca,
(SEQ ID NO: 5363)
AAGgcaaaaa,
(SEQ ID NO: 5364)
GUAguaaaua,
(SEQ ID NO: 5365)
CUAgugagag,
(SEQ ID NO: 5366)
GAAguuucug,
(SEQ ID NO: 5367)
CCUguacgua,
(SEQ ID NO: 5368)
GAGgugcgcg,
(SEQ ID NO: 5369)
AAGguguaaa,
(SEQ ID NO: 5370)
CCAguauguu,
(SEQ ID NO: 5371)
CCGgucagcu,
(SEQ ID NO: 5372)
AUGguuccug,
(SEQ ID NO: 5373)
CAAguuaaau,
(SEQ ID NO: 5374)
AGAguaggcu,
(SEQ ID NO: 5375)
AUGgugggca,
(SEQ ID NO: 5376)
GGAguaagac,
(SEQ ID NO: 5377)
AGGgucacga,
(SEQ ID NO: 5378)
UAGgugauau,
(SEQ ID NO: 5379)
GAAguaaguc,
(SEQ ID NO: 5380)
CGGguaagau,
(SEQ ID NO: 5381)
CAAguagcua,
(SEQ ID NO: 5382)
UGAguaaaau,
(SEQ ID NO: 5383)
GUCguacgug,
(SEQ ID NO: 5384)
AUGguacgua,
(SEQ ID NO: 5385)
CAGgucucgg,
(SEQ ID NO: 5386)
GAGgcauguc,
(SEQ ID NO: 5387)
AGAgugggau,
(SEQ ID NO: 5388)
GUGguuagag,
(SEQ ID NO: 5389)
UGGgugguga,
(SEQ ID NO: 5390)
AAGguuaaac,
(SEQ ID NO: 5391)
CUUguuagcu,
(SEQ ID NO: 5392)
AAAguaggaa,
(SEQ ID NO: 5393)
UAGguuguau,
(SEQ ID NO: 5394)
AGGgugcgcc,
(SEQ ID NO: 5395)
AAGgugggcu,
(SEQ ID NO: 5396)
UAAguaucug,
(SEQ ID NO: 5397)
AAGguaacgu,
(SEQ ID NO: 5398)
AUGguggggc,
(SEQ ID NO: 5399)
CAAguacacg,
(SEQ ID NO: 5400)
GGCguaagug,
(SEQ ID NO: 5401)
AUAguaggac,
(SEQ ID NO: 5402)
AGAgugaggu,
(SEQ ID NO: 5403)
UUUguaaaaa,
(SEQ ID NO: 5404)
GAAguuugua,
(SEQ ID NO: 5405)
CUAguaaucu,
(SEQ ID NO: 5406)
AAGguuuuua,
(SEQ ID NO: 5407)
GAGgugcguu,
(SEQ ID NO: 5408)
UAGgcgagua,
(SEQ ID NO: 5409)
ACCgugagua,
(SEQ ID NO: 5410)
CAGgucccga,
(SEQ ID NO: 5411)
AUGguacugg,
(SEQ ID NO: 5412)
UGAguucagu,
(SEQ ID NO: 5413)
AAUguguggu,
(SEQ ID NO: 5414)
UCCguugguu,
(SEQ ID NO: 5415)
CAGgucagag,
(SEQ ID NO: 5416)
CAGgucccua,
(SEQ ID NO: 5417)
UAGguagacu,
(SEQ ID NO: 5418)
CAAguuaagg,
(SEQ ID NO: 5419)
GAGgugugcg,
(SEQ ID NO: 5420)
GAAgcugccc,
(SEQ ID NO: 5421)
CGAguacgug,
(SEQ ID NO: 5422)
CGGguaggua,
(SEQ ID NO: 5423)
UUGguauuga,
(SEQ ID NO: 5424)
AUUguaugau,
(SEQ ID NO: 5425)
UUGguaugaa,
(SEQ ID NO: 5426)
GAGgugguca,
(SEQ ID NO: 5427)
GCUguaugaa,
(SEQ ID NO: 5428)
CAGguguugc,
(SEQ ID NO: 5429)
CAGguaaaac,
(SEQ ID NO: 5430)
AUAguaaggu,
(SEQ ID NO: 5431)
CUGguuagag,
(SEQ ID NO: 5432)
AGCgugugag,
(SEQ ID NO: 5433)
AAGguuaucu,
(SEQ ID NO: 5434)
CACgugagua,
(SEQ ID NO: 5435)
AGGgucagua,
(SEQ ID NO: 5436)
GAGguauaau,
(SEQ ID NO: 5437)
CAGguuauuu,
(SEQ ID NO: 5438)
AGGguggacu,
(SEQ ID NO: 5439)
AUUguaauuc,
(SEQ ID NO: 5440)
UUUguggguu,
(SEQ ID NO: 5441)
AUGguacgug,
(SEQ ID NO: 5442)
AAGguguucc,
(SEQ ID NO: 5443)
CAGgugacgc,
(SEQ ID NO: 5444)
GAGguacuaa,
(SEQ ID NO: 5445)
ACAguucagu,
(SEQ ID NO: 5446)
GAGgucacgg,
(SEQ ID NO: 5447)
CAAguaaggc,
(SEQ ID NO: 5448)
AAGguuuggg,
(SEQ ID NO: 5449)
AAAgugggcu,
(SEQ ID NO: 5450)
GCGguucuug,
(SEQ ID NO: 5451)
GAGguggagc,
(SEQ ID NO: 5452)
UGAgucagug,
(SEQ ID NO: 5453)
CAGgucaagg,
(SEQ ID NO: 5454)
AGUguaagcu,
(SEQ ID NO: 5455)
GAGgcagaaa,
(SEQ ID NO: 5456)
AAGgucacac,
(SEQ ID NO: 5457)
GAAguagguu,
(SEQ ID NO: 5458)
GUCguaaguu,
(SEQ ID NO: 5459)
AGAguaugca,
(SEQ ID NO: 5460)
CCUgugcaaa,
(SEQ ID NO: 5461)
ACGgugaaaa,
(SEQ ID NO: 5462)
CAGguacgaa,
(SEQ ID NO: 5463)
CAUgugagga,
(SEQ ID NO: 5464)
AGCgugagua,
(SEQ ID NO: 5465)
GGUguguagg,
(SEQ ID NO: 5466)
AACgugagcu,
(SEQ ID NO: 5467)
GAGgugaacu,
(SEQ ID NO: 5468)
AGAguucagu,
(SEQ ID NO: 5469)
AACgugugua,
(SEQ ID NO: 5470)
CAGguugugg,
(SEQ ID NO: 5471)
AAGguacuag,
(SEQ ID NO: 5472)
UCAgugaaaa,
(SEQ ID NO: 5473)
AAUgucuggu,
(SEQ ID NO: 5474)
ACGguaaaau,
(SEQ ID NO: 5475)
CUGguguaag,
(SEQ ID NO: 5476)
GAGgugcgaa,
(SEQ ID NO: 5477)
AGGguuucuc,
(SEQ ID NO: 5478)
CAGguagccc,
(SEQ ID NO: 5479)
AUUguauugg,
(SEQ ID NO: 5480)
AUGguacuua,
(SEQ ID NO: 5481)
GAGgcccgac,
(SEQ ID NO: 5482)
UCGguaagac,
(SEQ ID NO: 5483)
CGGgcuguag,
(SEQ ID NO: 5484)
UAUgugugug,
(SEQ ID NO: 5485)
UAGguagaaa,
(SEQ ID NO: 5486)
GUGgucauua,
(SEQ ID NO: 5487)
UAGgugaaag,
(SEQ ID NO: 5488)
ACUguaauuc,
(SEQ ID NO: 5489)
GCAguacagg,
(SEQ ID NO: 5490)
UCGgugaguc,
(SEQ ID NO: 5491)
UAUguaggga,
(SEQ ID NO: 5492)
AUGguauguc,
(SEQ ID NO: 5493)
GUGgugugug,
(SEQ ID NO: 5494)
CUGgugaccu,
(SEQ ID NO: 5495)
AAUgugaaua,
(SEQ ID NO: 5496)
UAGgucucac,
(SEQ ID NO: 5497)
GAGguuauug,
(SEQ ID NO: 5498)
UGAguaggcu,
(SEQ ID NO: 5499)
CGGgcacgua,
(SEQ ID NO: 5500)
GCAguaaaua,
(SEQ ID NO: 5501)
CCGgugagag,
(SEQ ID NO: 5502)
UAAguugguc,
(SEQ ID NO: 5503)
CCGgugagcc,
(SEQ ID NO: 5504)
AAGguuguca,
(SEQ ID NO: 5505)
CUGguauuau,
(SEQ ID NO: 5506)
GGGguauggg,
(SEQ ID NO: 5507)
AAAgucagua,
(SEQ ID NO: 5508)
UUUguaugua,
(SEQ ID NO: 5509)
UAAguacugc,
(SEQ ID NO: 5510)
CAGguaccaa,
(SEQ ID NO: 5511)
GAAguucaga,
(SEQ ID NO: 5512)
AUGgugcggu,
(SEQ ID NO: 5513)
GUGgugaggu,
(SEQ ID NO: 5514)
UGAguaagcc,
(SEQ ID NO: 5515)
UAUguaaggg,
(SEQ ID NO: 5516)
GUGguggaaa,
(SEQ ID NO: 5517)
GAGgugauug,
(SEQ ID NO: 5518)
GGAguuugua,
(SEQ ID NO: 5519)
AAGgucacga,
(SEQ ID NO: 5520)
GUGguagagg,
(SEQ ID NO: 5521)
UAAguauauc,
(SEQ ID NO: 5522)
AAGgugucca,
(SEQ ID NO: 5523)
UAUgugguau,
(SEQ ID NO: 5524)
GAGguacaau,
(SEQ ID NO: 5525)
AAGguggggg,
(SEQ ID NO: 5526)
GGAguaggug,
and
(SEQ ID NO: 5527)
UAGgugacuu.

In some embodiments, the splice site sequence (e.g., 5′ splice site sequence) comprises AGA. In some embodiments, the splice site sequence (e.g., 5′ splice site sequence) comprises AAA. In some embodiments, the splice site sequence (e.g., 5′ splice site sequence) comprises AAC. In some embodiments, the splice site sequence (e.g., 5′ splice site sequence) comprises AAU. In some embodiments, the splice site sequence (e.g., 5′ splice site sequence) comprises AAG. In some embodiments, the splice site sequence (e.g., 5′ splice site sequence) comprises ACA. In some embodiments, the splice site sequence (e.g., 5′ splice site sequence) comprises AUA. In some embodiments, the splice site sequence (e.g., 5′ splice site sequence) comprises AUU. In some embodiments, the splice site sequence (e.g., 5′ splice site sequence) comprises AUG. In some embodiments, the splice site sequence (e.g., 5′ splice site sequence) comprises AUC. In some embodiments, the splice site sequence (e.g., 5′ splice site sequence) comprises CAA. In some embodiments, the splice site sequence (e.g., 5′ splice site sequence) comprises CAU. In some embodiments, the splice site sequence (e.g., 5′ splice site sequence) comprises CAC. In some embodiments, the splice site sequence (e.g., 5′ splice site sequence) comprises CAG. In some embodiments, the splice site sequence (e.g., 5′ splice site sequence) comprises GAA. In some embodiments, the splice site sequence (e.g., 5′ splice site sequence) comprises GAC. In some embodiments, the splice site sequence (e.g., 5′ splice site sequence) comprises GAU. In some embodiments, the splice site sequence (e.g., 5′ splice site sequence) comprises GAG. In some embodiments, the splice site sequence (e.g., 5′ splice site sequence) comprises GGA. In some embodiments, the splice site sequence (e.g., 5′ splice site sequence) comprises GCA. In some embodiments, the splice site sequence (e.g., 5′ splice site sequence) comprises GGG. In some embodiments, the splice site sequence (e.g., 5′ splice site sequence) comprises GGC. In some embodiments, the splice site sequence (e.g., 5′ splice site sequence) comprises GUU. In some embodiments, the splice site sequence (e.g., 5′ splice site sequence) comprises GGU. In some embodiments, the splice site sequence (e.g., 5′ splice site sequence) comprises GUC. In some embodiments, the splice site sequence (e.g., 5′ splice site sequence) comprises GUA. In some embodiments, the splice site sequence (e.g., 5′ splice site sequence) comprises GUG. In some embodiments, the splice site sequence (e.g., 5′ splice site sequence) comprises UCU. In some embodiments, the splice site sequence (e.g., 5′ splice site sequence) comprises UCC. In some embodiments, the splice site sequence (e.g., 5′ splice site sequence) comprises UCA. In some embodiments, the splice site sequence (e.g., 5′ splice site sequence) comprises UCG. In some embodiments, the splice site sequence (e.g., 5′ splice site sequence) comprises UUU. In some embodiments, the splice site sequence (e.g., 5′ splice site sequence) comprises UUC. In some embodiments, the splice site sequence (e.g., 5′ splice site sequence) comprises UUA. In some embodiments, the splice site sequence (e.g., 5′ splice site sequence) comprises UUG. In some embodiments, the splice site sequence (e.g., 5′ splice site sequence) comprises UGU. In some embodiments, the splice site sequence (e.g., 5′ splice site sequence) comprises UAU. In some embodiments, the splice site sequence (e.g., 5′ splice site sequence) comprises GGA. In some embodiments, the splice site sequence (e.g., 5′ splice site sequence) comprises CUU. In some embodiments, the splice site sequence (e.g., 5′ splice site sequence) comprises CUC. In some embodiments, the splice site sequence (e.g., 5′ splice site sequence) comprises CUA. In some embodiments, the splice site sequence (e.g., 5′ splice site sequence) comprises CUG. In some embodiments, the splice site sequence (e.g., 5′ splice site sequence) comprises CCU. In some embodiments, the splice site sequence (e.g., 5′ splice site sequence) comprises CCC. In some embodiments, the splice site sequence (e.g., 5′ splice site sequence) comprises CCA. In some embodiments, the splice site sequence (e.g., 5′ splice site sequence) comprises CCG. In some embodiments, the splice site sequence (e.g., 5′ splice site sequence) comprises ACU. In some embodiments, the splice site sequence (e.g., 5′ splice site sequence) comprises ACC. In some embodiments, the splice site sequence (e.g., 5′ splice site sequence) comprises ACG. In some embodiments, the splice site sequence (e.g., 5′ splice site sequence) comprises AGC. In some embodiments, the splice site sequence (e.g., 5′ splice site sequence) comprises AGU. In some embodiments, the splice site sequence (e.g., 5′ splice site sequence) comprises AGG. In some embodiments, the splice site sequence (e.g., 5′ splice site sequence) comprises CGU. In some embodiments, the splice site sequence (e.g., 5′ splice site sequence) comprises UAC. In some embodiments, the splice site sequence (e.g., 5′ splice site sequence) comprises UAA. In some embodiments, the splice site sequence (e.g., 5′ splice site sequence) comprises UAG. In some embodiments, the splice site sequence (e.g., 5′ splice site sequence) comprises CGC. In some embodiments, the splice site sequence (e.g., 5′ splice site sequence) comprises CGA. In some embodiments, the splice site sequence (e.g., 5′ splice site sequence) comprises CGG. In some embodiments, the splice site sequence comprises AGAguaaggg.

In an embodiment, a gene sequence or splice site sequence provided herein is related to a proliferative disease, disorder, or condition (e.g., cancer, benign neoplasm, or inflammatory disease). In an embodiment, a gene sequence or splice site sequence provided herein is related to a non-proliferative disease, disorder, or condition. In an embodiment, a gene sequence or splice site sequence provided herein is related to a neurological disease or disorder; autoimmune disease or disorder; immunodeficiency disease or disorder; lysosomal storage disease or disorder; cardiovascular condition, disease or disorder; metabolic disease or disorder; respiratory condition, disease, or disorder; renal disease or disorder; or infectious disease in a subject. In an embodiment, a gene sequence or splice site sequence provided herein is related to a neurological disease or disorder (e.g., Huntington's disease). In an embodiment, a gene sequence or splice site sequence provided herein is related to an immunodeficiency disease or disorder. In an embodiment, a gene sequence or splice site sequence provided herein is related to a lysosomal storage disease or disorder. In an embodiment, a gene sequence or splice site sequence provided herein is related to a cardiovascular condition, disease or disorder. In an embodiment, a gene sequence or splice site sequence provided herein is related to a metabolic disease or disorder. In an embodiment, a gene sequence or splice site sequence provided herein is related to a respiratory condition, disease, or disorder. In an embodiment, a gene sequence or splice site sequence provided herein is related to a renal disease or disorder. In an embodiment, a gene sequence or splice site sequence provided herein is related to an infectious disease.

In an embodiment, a gene sequence or splice site sequence provided herein is related to a mental retardation disorder. In an embodiment, a gene sequence or splice site sequence provided herein is related to a mutation in the SETD5 gene. In an embodiment, a gene sequence or splice site sequence provided herein is related to an immunodeficiency disorder. In an embodiment, a gene sequence and splice site sequence provided herein is related to a mutation in the GATA2 gene. In an embodiment, a gene sequence or splice site sequence provided herein is related to a lysosomal storage disease.

In some embodiments, a compound of Formula (I), (II), (III), or (IV) described herein interacts with (e.g., binds to) a splicing complex component (e.g., a nucleic acid (e.g., an RNA) or a protein). In some embodiments, the splicing complex component is selected from 9G8, A1 hnRNP, A2 hnRNP, ASD-1, ASD-2b, ASF, BRR2, B1 hnRNP, C1 hnRNP, C2 hnRNP, CBP20, CBP80, CELF, F hnRNP, FBP11, Fox-1, Fox-2, G hnRNP, H hnRNP, hnRNP 1, hnRNP 3, hnRNP C, hnRNP G, hnRNP K, hnRNP M, hnRNP U, Hu, HUR, I hnRNP, K hnRNP, KH-type splicing regulatory protein (KSRP), L hnRNP, LUC7L, M hnRNP, mBBP, muscle-blind like (MBNL), NF45, NFAR, Nova-1, Nova-2, nPTB, P54/SFRS11, polypyrimidine tract binding protein (PTB), a PRP protein (e.g., PRP8, PRP6, PRP31, PRP4, PRP3, PRP28, PRP5, PRP2, PRP19), PRP19 complex proteins, RBM42, R hnRNP, RNPC1, SAD1, SAM68, SC35, SF, SF1/BBP, SF2, SF3A complex, SF3B complex, SFRS10, an Sm protein (such as B, D1, D2, D3, F, E, G), SNU17, SNU66, SNU114, an SR protein, SRm300, SRp20, SRp30c, SRP35C, SRP36, SRP38, SRp40, SRp55, SRp75, SRSF, STAR, GSG, SUP-12, TASR-1, TASR-2, TIA, TIAR, TRA2, TRA2a/b, U hnRNP, U1 snRNP, U11 snRNP, U12 snRNP, U1-70K, U1-A, U1-C, U2 snRNP, U2AF1-RS2, U2AF35, U2AF65, U4 snRNP, U5 snRNP, U6 snRNP, Urp, and YB1.

In some embodiments, the splicing complex component comprises RNA (e.g., snRNA). In some embodiments, a compound described herein binds to a splicing complex component comprising snRNA. The snRNA may be selected from, e.g., U1 snRNA, U2 snRNA, U4 snRNA, U5 snRNA, U6 snRNA, U11 snRNA, U12 snRNA, U4atac snRNA, and any combination thereof.

In some embodiments, the splicing complex component comprises a protein, e.g., a protein associated with an snRNA. In some embodiments, the protein comprises SC35, SRp55, SRp40, SRm300, SFRS10, TASR-1, TASR-2, SF2/ASF, 9G8, SRp75, SRp30c, SRp20 and P54/SFRS11. In some embodiments, the splicing complex component comprises a U2 snRNA auxiliary factor (e.g., U2AF65, U2AF35), Urp/U2AF1-RS2, SF1/BBP, CBP80, CBP 20, SF1 or PTB/hnRNP1. In some embodiments, the hnRNP protein comprises A1, A2/B1, L, M, K, U, F, H, G, R, I or C1/C2. Human genes encoding hnRNPs include HNRNPA0, HNRNPA1, HNRNPA1L1, HNRNPA1L2, HNRNPA3, HNRNPA2B1, HNRNPAB, HNRNPB1, HNRNPC, HNRNPCL1, HNRNPD, HNRPDL, HNRNPF, HNRNPH1, HNRNPH2, HNRNPH3, HNRNPK, HNRNPL, HNRPLL, HNRNPM, HNRNPR, HNRNPU, HNRNPUL1, HNRNPUL2, HNRNPUL3, and FMR1.

In one aspect, the compounds of Formula (I), (II), (III), or (IV) and pharmaceutically acceptable salts, solvates, hydrates, tautomers, stereoisomers, and compositions thereof, may modulate (e.g., increase or decrease) a splicing event of a target nucleic acid sequence (e.g., DNA, RNA, or a pre-mRNA), for example, a nucleic acid encoding a gene described herein, or a nucleic acid encoding a protein described herein, or a nucleic acid comprising a splice site described herein. In an embodiment, the splicing event is an alternative splicing event.

In an embodiment, the compound of Formula (I), (II), (III), or (IV) or a pharmaceutically acceptable salt, solvate, hydrate, tautomer, stereoisomer, and compositions thereof increases splicing at splice site on a target nucleic acid (e.g., an RNA, e.g., a pre-mRNA), by about 0.5%, 1%, 2%, 3%, 4%, 5%, 6%, 7%, 8%, 9%, 10%, 15%, 20%, 25%, 30%, 35%, 40%, 45%, 50%, 55%, 60%, 65%, 70%, 75%, 80%, 85%, 90%, 95%, or more, e.g., as determined by a known method in the art, e.g., qPCR. In an embodiment, the compound of Formula (I), (II), (III), or (IV) or a pharmaceutically acceptable salt, solvate, hydrate, tautomer, stereoisomer, and compositions thereof decreases splicing at splice site on a target nucleic acid (e.g., an RNA, e.g., a pre-mRNA), by about 0.5%, 1%, 2%, 3%, 4%, 5%, 6%, 7%, 8%, 9%, 10%, 15%, 20%, 25%, 30%, 35%, 40%, 45%, 50%, 55%, 60%, 65%, 70%, 75%, 80%, 85%, 90%, 95%, or more, e.g., as determined by a known method in the art, e.g., qPCR.

In another aspect, the present disclosure features a method of forming a complex comprising a component of a spliceosome (e.g., a major spliceosome component or a minor spliceosome component), a nucleic acid (e.g., a DNA, RNA, e.g., a pre-mRNA), and a compound of Formula (I), (II), (III), or (IV) or a pharmaceutically acceptable salt, solvate, hydrate, tautomer, stereoisomer, or composition thereof, comprising contacting the nucleic acid (e.g., a DNA, RNA, e.g., a pre-mRNA) with said compound of Formula (I), (II), (III), or (IV). In an embodiment, the component of a spliceosome is selected from the U1, U2, U4, U5, U6, U11, U12, U4atac, U6atac small nuclear ribonucleoproteins (snRNPs), or a related accessory factor. In an embodiment, the component of a spliceosome is recruited to the nucleic acid in the presence of the compound of Formula (I), (II), (III), or (IV), or a pharmaceutically acceptable salt, solvate, hydrate, tautomer, stereoisomer, or composition thereof.

In another aspect, the present disclosure features a method of altering the conformation of a nucleic acid (e.g., a DNA, RNA, e.g., a pre-mRNA) comprising contacting the nucleic acid with a compound of Formula (I), (II), (III), or (IV) or a pharmaceutically acceptable salt, solvate, hydrate, tautomer, stereoisomer, or composition thereof. In an embodiment, the altering comprises forming a bulge or kink in the nucleic acid. In an embodiment, the altering comprises stabilizing a bulge or a kink in the nucleic acid. In an embodiment, the altering comprises reducing a bulge or a kink in the nucleic acid. In an embodiment, the nucleic acid comprises a splice site. In an embodiment, the compound of Formula (I), (II), (III), or (IV) interacts with a nucleobase, ribose, or phosphate moiety of a nucleic acid (e.g., a DNA, RNA, e.g., pre-mRNA).

The present disclosure also provides methods for the treatment or prevention of a disease, disorder, or condition. In an embodiment, the disease, disorder or condition is related to (e.g., caused by) a splicing event, such as an unwanted, aberrant, or alternative splicing event. In an embodiment, the disease, disorder or condition comprises a proliferative disease (e.g., cancer, benign neoplasm, or inflammatory disease) or non-proliferative disease. In an embodiment, the disease, disorder, or condition comprises a neurological disease, autoimmune disorder, immunodeficiency disorder, cardiovascular condition, metabolic disorder, lysosomal storage disease, respiratory condition, renal disease, or infectious disease in a subject. In another embodiment, the disease, disorder, or condition comprises a haploinsufficiency disease, an autosomal recessive disease (e.g., with residual function), or a paralogue activation disorder. In another embodiment, the disease, disorder, or condition comprises an autosomal dominant disorder (e.g., with residual function). Such methods comprise the step of administering to the subject in need thereof an effective amount of a compound of Formula (I), (II), (III), (IV), or a pharmaceutically acceptable salt, solvate, hydrate, tautomer, stereoisomer thereof, or a pharmaceutical composition thereof. In certain embodiments, the methods described herein include administering to a subject an effective amount of a compound of Formula (I), (II), (III), (IV), or a pharmaceutically acceptable salt thereof, or a pharmaceutical composition thereof.

In certain embodiments, the subject being treated is a mammal. In certain embodiments, the subject is a human. In certain embodiments, the subject is a domesticated animal, such as a dog, cat, cow, pig, horse, sheep, or goat. In certain embodiments, the subject is a companion animal such as a dog or cat. In certain embodiments, the subject is a livestock animal such as a cow, pig, horse, sheep, or goat. In certain embodiments, the subject is a zoo animal. In another embodiment, the subject is a research animal such as a rodent, dog, or non-human primate. In certain embodiments, the subject is a non-human transgenic animal such as a transgenic mouse or transgenic pig.

A proliferative disease may also be associated with inhibition of apoptosis of a cell in a biological sample or subject. All types of biological samples described herein or known in the art are contemplated as being within the scope of the disclosure. The compounds of Formula (I), (II), (III), or (IV) and pharmaceutically acceptable salts, solvates, hydrates, tautomers, stereoisomers, and compositions thereof, may induce apoptosis, and therefore, be useful in treating and/or preventing proliferative diseases.

In certain embodiments, the proliferative disease to be treated or prevented using the compounds of Formula (I), (II), (III), or (IV) is cancer. As used herein, the term “cancer” refers to a malignant neoplasm (Stedman's Medical Dictionary, 25th ed.; Hensyl ed.; Williams & Wilkins: Philadelphia, 1990). All types of cancers disclosed herein or known in the art are contemplated as being within the scope of the disclosure. Exemplary cancers include, but are not limited to, acoustic neuroma; adenocarcinoma; adrenal gland cancer; anal cancer; angiosarcoma (e.g., lymphangiosarcoma, lymphangioendotheliosarcoma, hemangiosarcoma); appendix cancer; benign monoclonal gammopathy; biliary cancer (e.g., cholangiocarcinoma); bladder cancer; breast cancer (e.g., adenocarcinoma of the breast, papillary carcinoma of the breast, mammary cancer, medullary carcinoma of the breast); brain cancer (e.g., meningioma, glioblastomas, glioma (e.g., astrocytoma, oligodendroglioma), medulloblastoma); bronchus cancer; carcinoid tumor; cervical cancer (e.g., cervical adenocarcinoma); choriocarcinoma; chordoma; craniopharyngioma; colorectal cancer (e.g., colon cancer, rectal cancer, colorectal adenocarcinoma); connective tissue cancer; epithelial carcinoma; ependymoma; endotheliosarcoma (e.g., Kaposi's sarcoma, multiple idiopathic hemorrhagic sarcoma); endometrial cancer (e.g., uterine cancer, uterine sarcoma); esophageal cancer (e.g., adenocarcinoma of the esophagus, Barrett's adenocarcinoma); Ewing's sarcoma; eye cancer (e.g., intraocular melanoma, retinoblastoma); familiar hypereosinophilia; gall bladder cancer; gastric cancer (e.g., stomach adenocarcinoma); gastrointestinal stromal tumor (GIST); germ cell cancer; head and neck cancer (e.g., head and neck squamous cell carcinoma, oral cancer (e.g., oral squamous cell carcinoma), throat cancer (e.g., laryngeal cancer, pharyngeal cancer, nasopharyngeal cancer, oropharyngeal cancer)); hematopoietic cancers (e.g., leukemia such as acute lymphocytic leukemia (ALL) (e.g., B-cell ALL, T-cell ALL), acute myelocytic leukemia (AML) (e.g., B-cell AML, T-cell AML), chronic myelocytic leukemia (CML) (e.g., B-cell CML, T-cell CML), and chronic lymphocytic leukemia (CLL) (e.g., B-cell CLL, T-cell CLL)); lymphoma such as Hodgkin lymphoma (HL) (e.g., B-cell HL, T-cell HL) and non-Hodgkin lymphoma (NHL) (e.g., B-cell NHL such as diffuse large cell lymphoma (DLCL) (e.g., diffuse large B-cell lymphoma), follicular lymphoma, chronic lymphocytic leukemia/small lymphocytic lymphoma (CLL/SLL), mantle cell lymphoma (MCL), marginal zone B-cell lymphomas (e.g., mucosa-associated lymphoid tissue (MALT) lymphomas, nodal marginal zone B-cell lymphoma, splenic marginal zone B-cell lymphoma), primary mediastinal B-cell lymphoma, Burkitt lymphoma, lymphoplasmacytic lymphoma (i.e., Waldenstrom's macroglobulinemia), hairy cell leukemia (HCL), immunoblastic large cell lymphoma, precursor B-lymphoblastic lymphoma and primary central nervous system (CNS) lymphoma; and T-cell NHL such as precursor T-lymphoblastic lymphoma/leukemia, peripheral T-cell lymphoma (PTCL) (e.g., cutaneous T-cell lymphoma (CTCL) (e.g., mycosis fungoides, Sezary syndrome), angioimmunoblastic T-cell lymphoma, extranodal natural killer T-cell lymphoma, enteropathy type T-cell lymphoma, subcutaneous panniculitis-like T-cell lymphoma, and anaplastic large cell lymphoma); a mixture of one or more leukemia/lymphoma as described above; and multiple myeloma (MM)), heavy chain disease (e.g., alpha chain disease, gamma chain disease, mu chain disease); hemangioblastoma; hypopharynx cancer; inflammatory myofibroblastic tumors; immunocytic amyloidosis; kidney cancer (e.g., nephroblastoma a.k.a. Wilms' tumor, renal cell carcinoma); liver cancer (e.g., hepatocellular cancer (HCC), malignant hepatoma); lung cancer (e.g., bronchogenic carcinoma, small cell lung cancer (SCLC), non-small cell lung cancer (NSCLC), adenocarcinoma of the lung); leiomyosarcoma (LMS); mastocytosis (e.g., systemic mastocytosis); muscle cancer; myelodysplastic syndrome (MDS); mesothelioma; myeloproliferative disorder (MPD) (e.g., polycythemia vera (PV), essential thrombocytosis (ET), agnogenic myeloid metaplasia (AMM) a.k.a. myelofibrosis (MF), chronic idiopathic myelofibrosis, chronic myelocytic leukemia (CML), chronic neutrophilic leukemia (CNL), hypereosinophilic syndrome (HES)); neuroblastoma; neurofibroma (e.g., neurofibromatosis (NF) type 1 or type 2, schwannomatosis); neuroendocrine cancer (e.g., gastroenteropancreatic neuroendocrine tumor (GEP-NET), carcinoid tumor); osteosarcoma (e.g., bone cancer); ovarian cancer (e.g., cystadenocarcinoma, ovarian embryonal carcinoma, ovarian adenocarcinoma); papillary adenocarcinoma; pancreatic cancer (e.g., pancreatic adenocarcinoma, intraductal papillary mucinous neoplasm (IPMN), Islet cell tumors); penile cancer (e.g., Paget's disease of the penis and scrotum); pinealoma; primitive neuroectodermal tumor (PNT); plasma cell neoplasia; paraneoplastic syndromes; intraepithelial neoplasms; prostate cancer (e.g., prostate adenocarcinoma); rectal cancer; rhabdomyosarcoma; salivary gland cancer; skin cancer (e.g., squamous cell carcinoma (SCC), keratoacanthoma (KA), melanoma, basal cell carcinoma (BCC)); small bowel cancer (e.g., appendix cancer); soft tissue sarcoma (e.g., malignant fibrous histiocytoma (MFH), liposarcoma, malignant peripheral nerve sheath tumor (MPNST), chondrosarcoma, fibrosarcoma, myxosarcoma); sebaceous gland carcinoma; small intestine cancer; sweat gland carcinoma; synovioma; testicular cancer (e.g., seminoma, testicular embryonal carcinoma); thyroid cancer (e.g., papillary carcinoma of the thyroid, papillary thyroid carcinoma (PTC), medullary thyroid cancer); urethral cancer; vaginal cancer; and vulvar cancer (e.g., Paget's disease of the vulva).

In some embodiments, the proliferative disease is associated with a benign neoplasm. For example, a benign neoplasm may include adenoma, fibroma, hemangioma, tuberous sclerosis, and lipoma. All types of benign neoplasms disclosed herein or known in the art are contemplated as being within the scope of the disclosure.

In some embodiments, the proliferative disease is associated with angiogenesis. All types of angiogenesis disclosed herein or known in the art are contemplated as being within the scope of the disclosure.

In some embodiments, the compound of Formula (I), (II), (III), or (IV), or a pharmaceutically acceptable salt thereof, or compositions comprising such compound or pharmaceutically acceptable salt thereof, is used to prevent or treat a non-proliferative disease. Exemplary non-proliferative diseases include a neurological disease, autoimmune disorder, immunodeficiency disorder, lysosomal storage disease, cardiovascular condition, metabolic disorder, respiratory condition, inflammatory disease, renal disease, or infectious disease.

In certain embodiments, the non-proliferative disease is a neurological disease. In certain embodiments, the compound of Formula (I), (II), (III), or (IV), or a pharmaceutically acceptable salt thereof, or compositions comprising such compound or pharmaceutically acceptable salt thereof, is used to prevent or treat a neurological disease, disorder, or condition. A neurological disease, disorder, or condition may include a neurodegenerative disease, a psychiatric condition, or a musculoskeletal disease. A neurological disease may further include a repeat expansion disease, e.g., which may be characterized by the expansion of a nucleic acid sequence in the genome. For example, a repeat expansion disease includes myotonic dystrophy, amyotrophic lateral sclerosis, Huntington's disease, a trinucleotide repeat disease, or a polyglutamine disorder (e.g., ataxia, fragile X syndrome). In some embodiments, the neurological disease comprises a repeat expansion disease, e.g., Huntington's disease. Additional neurological diseases, disorders, and conditions include Alzheimer's disease, Huntington's chorea, a prion disease (e.g., Creutzfeld-Jacob disease, bovine spongiform encephalopathy, Kuru, or scrapie), a mental retardation disorder (e.g., a disorder caused by a SETD5 gene mutation, e.g., intellectual disability-facial dysmorphism syndrome, autism spectrum disorder), Lewy Body disease, diffuse Lewy body disease (DLBD), dementia, progressive supranuclear palsy (PSP), progressive bulbar palsy (PBP), psuedobulbar palsy, spinal and bulbar muscular atrophy (SBMA), primary lateral sclerosis, Pick's disease, primary progressive aphasia, corticobasal dementia, Parkinson's disease, Down's syndrome, multiple system atrophy, spinal muscular atrophy (SMA), progressive spinobulbar muscular atrophy (e.g., Kennedy disease), post-polio syndrome (PPS), spinocerebellar ataxia, pantothenate kinase-associated neurodegeneration (PANK), spinal degenerative disease/motor neuron degenerative diseases, upper motor neuron disorder, lower motor neuron disorder, Hallervorden-Spatz syndrome, cerebral infarction, cerebral trauma, chronic traumatic encephalopathy, transient ischemic attack, Lytigo-bodig (amyotrophic lateral sclerosis-parkinsonism dementia), Guam-Parkinsonism dementia, hippocampal sclerosis, corticobasal degeneration, Alexander disease, Apler's disease, Krabbe's disease, neuroborreliosis, neurosyphilis, Sandhoff disease, Tay-Sachs disease, Schilder's disease, Batten disease, Cockayne syndrome, Kearns-Sayre syndrome, Gerstmann-Straussler-Scheinker syndrome and other transmissible spongiform encephalopathies, hereditary spastic paraparesis, Leigh's syndrome, a demyelinating diseases, neuronal ceroid lipofuscinoses, epilepsy, tremors, depression, mania, anxiety and anxiety disorders, sleep disorders (e.g., narcolepsy, fatal familial insomnia), acute brain injuries (e.g., stroke, head injury), autism, Machado-Joseph disease, or a combination thereof. In some embodiments, the neurological disease comprises Friedrich's ataxia or Sturge Weber syndrome. In some embodiments, the neurological disease comprises Huntington's disease. In some embodiments, the neurological disease comprises spinal muscular atrophy. All types of neurological diseases disclosed herein or known in the art are contemplated as being within the scope of the disclosure.

In certain embodiments, the non-proliferative disease is an autoimmune disorder or an immunodeficiency disorder. In certain embodiments, the compound of Formula (I), (II), (III), or (IV), or a pharmaceutically acceptable salt thereof, or compositions comprising such compound or pharmaceutically acceptable salt thereof, is used to prevent or treat an autoimmune disease, disorder, or condition, or an immunodeficiency disease, disorder, or condition. Exemplary autoimmune and immunodeficiency diseases, disorders, and conditions include arthritis (e.g., rheumatoid arthritis, osteoarthritis, gout), Chagas disease, chronic obstructive pulmonary disease (COPD), dermatomyositis, diabetes mellitus type 1, endometriosis, Goodpasture's syndrome, Graves' disease, Guillain-Barre syndrome (GBS), Hashiomoto's disease, Hidradenitis suppurativa, Kawasaki disease, ankylosing spondylitis, IgA nephropathy, idiopathic thrombocytopenic purpura, inflammatory bowel disease, Crohn's disease, ulcerative colitis, collagenous colitis, lymphocytic colitis, ischemic colitis, diversion colitis, Behcet's syndrome, infective colitis, indeterminate colitisinterstitial cystitis, lupus (e.g., systemic lupus erythematosus, discoid lupus, drug-induced lupus, neonatal lupus), mixed connective tissue disease, morphea, multiple sclerosis, myasthenia gravis, narcolepsy, neuromyotonia, pemphigus vulgaris, pernicious anemia, psoriasis, psoriatic arthritis, polymyositis, primary biliary cirrhosis, relapsing polychondritis, scleroderma, Sjögren's syndrome, Stiff person syndrome, vasculitis, vitiligo, a disorder caused by a GATA2 mutation (e.g., GATA2 deficiency; GATA2 haploinsufficiency; Emberger syndrome; monocytopenia and Mycobacterium avium complex/dendritic cell, monocyte, B and NK lymphocyte deficiency; familial myelodysplastic syndrome; acute myeloid leukemia; chronic myelomonocytic leukemia), neutropenia, aplastic anemia, and Wegener's granulomatosis. In some embodiments, the autoimmune or immunodeficiency disorder comprises chronic mucocutaneous candidiasis. All types of autoimmune disorders and immunodeficiency disorders disclosed herein or known in the art are contemplated as being within the scope of the disclosure.

In certain embodiments, the non-proliferative disease is a cardiovascular condition. In certain embodiments, the compound of Formula (I), (II), (III), or (IV), or a pharmaceutically acceptable salt thereof, or compositions comprising such compound or pharmaceutically acceptable salt thereof, is used to prevent or treat a cardiovascular disease, disorder, or condition. A cardiovascular disease, disorder, or condition may include a condition relating to the heart or vascular system, such as the arteries, veins, or blood. Exemplary cardiovascular diseases, disorders, or conditions include angina, arrhythmias (atrial or ventricular or both), heart failure, arteriosclerosis, atheroma, atherosclerosis, cardiac hypertrophy, cardiac or vascular aneurysm, cardiac myocyte dysfunction, carotid obstructive disease, endothelial damage after PTCA (percutaneous transluminal coronary angioplasty), hypertension including essential hypertension, pulmonary hypertension and secondary hypertension (renovascular hypertension, chronic glomerulonephritis), myocardial infarction, myocardial ischemia, peripheral obstructive arteriopathy of a limb, an organ, or a tissue; peripheral artery occlusive disease (PAOD), reperfusion injury following ischemia of the brain, heart or other organ or tissue, restenosis, stroke, thrombosis, transient ischemic attack (TIA), vascular occlusion, vasculitis, and vasoconstriction. All types of cardiovascular diseases, disorders, or conditions disclosed herein or known in the art are contemplated as being within the scope of the disclosure.

In certain embodiments, the non-proliferative disease is a metabolic disorder. In certain embodiments, the compound of Formula (I), (II), (III), or (IV), or a pharmaceutically acceptable salt thereof, or compositions comprising such compound or pharmaceutically acceptable salt thereof, is used to prevent or treat a metabolic disease, disorder, or condition. A metabolic disease, disorder, or condition may include a disorder or condition that is characterized by abnormal metabolism, such as those disorders relating to the consumption of food and water, digestion, nutrient processing, and waste removal. A metabolic disease, disorder, or condition may include an acid-base imbalance, a mitochondrial disease, a wasting syndrome, a malabsorption disorder, an iron metabolism disorder, a calcium metabolism disorder, a DNA repair deficiency disorder, a glucose metabolism disorder, hyperlactatemia, a disorder of the gut microbiota. Exemplary metabolic conditions include obesity, diabetes (Type I or Type II), insulin resistance, glucose intolerance, lactose intolerance, eczema, hypertension, Hunter syndrome, Krabbe disease, sickle cell anemia, maple syrup urine disease, Pompe disease, and metachromatic leukodystrophy. All types of metabolic diseases, disorders, or conditions disclosed herein or known in the art are contemplated as being within the scope of the disclosure.

In certain embodiments, the non-proliferative disease is a respiratory condition. In certain embodiments, the compound of Formula (I), (II), (III), or (IV), or a pharmaceutically acceptable salt thereof, or compositions comprising such compound or pharmaceutically acceptable salt thereof, is used to prevent or treat a respiratory disease, disorder, or condition. A respiratory disease, disorder, or condition can include a disorder or condition relating to any part of the respiratory system, such as the lungs, alveoli, trachea, bronchi, nasal passages, or nose. Exemplary respiratory diseases, disorders, or conditions include asthma, allergies, bronchitis, allergic rhinitis, chronic obstructive pulmonary disease (COPD), lung cancer, oxygen toxicity, emphysema, chronic bronchitis, and acute respiratory distress syndrome. All types of respiratory diseases, disorders, or conditions disclosed herein or known in the art are contemplated as being within the scope of the disclosure.

In certain embodiments, the non-proliferative disease is a renal disease. In certain embodiments, the compound of Formula (I), (II), (III), or (IV), or a pharmaceutically acceptable salt thereof, or compositions comprising such compound or pharmaceutically acceptable salt thereof, is used to prevent or treat a renal disease, disorder, or condition. A renal disease, disorder, or condition can include a disease, disorder, or condition relating to any part of the waste production, storage, and removal system, including the kidneys, ureter, bladder, urethra, adrenal gland, and pelvis. Exemplary renal diseases include acute kidney failure, amyloidosis, Alport syndrome, adenovirus nephritis, acute lobar nephronia, tubular necrosis, glomerulonephritis, kidney stones, urinary tract infections, chronic kidney disease, polycystic kidney disease, and focal segmental glomerulosclerosis (FSGS). In some embodiments, the renal disease, disorder, or condition comprises HIV-associated nephropathy or hypertensive nephropathy. All types of renal diseases, disorders, or conditions disclosed herein or known in the art are contemplated as being within the scope of the disclosure.

In certain embodiments, the non-proliferative disease is an infectious disease. In certain embodiments, the compound of Formula (I), (II), (III), or (IV), or a pharmaceutically acceptable salt thereof, or compositions comprising such compound or pharmaceutically acceptable salt thereof, is used to prevent or treat an infectious disease, disorder, or condition. An infectious disease may be caused by a pathogen such as a virus or bacteria. Exemplary infectious diseases include human immunodeficiency syndrome (HIV), acquired immunodeficiency syndrome (AIDS), meningitis, African sleeping sickness, actinomycosis, pneumonia, botulism, chlamydia, Chagas disease, Colorado tick fever, cholera, typhus, giardiasis, food poisoning, ebola hemorrhagic fever, diphtheria, Dengue fever, gonorrhea, streptococcal infection (e.g., Group A or Group B), hepatitis A, hepatitis B, hepatitis C, herpes simplex, hookworm infection, influenza, Epstein-Barr infection, Kawasaki disease, kuru, leprosy, leishmaniasis, measles, mumps, norovirus, meningococcal disease, malaria, Lyme disease, listeriosis, rabies, rhinovirus, rubella, tetanus, shingles, scarlet fever, scabies, Zika fever, yellow fever, tuberculosis, toxoplasmosis, or tularemia. In some embodiments, the infectious disease comprises cytomegalovirus. All types of infectious diseases, disorders, or conditions disclosed herein or known in the art are contemplated as being within the scope of the disclosure.

In certain embodiments, the disease, disorder, or condition is a haploinsufficiency disease. In certain embodiments, the compound of Formula (I), (II), (III), or (IV), or a pharmaceutically acceptable salt thereof, or compositions comprising such compound or pharmaceutically acceptable salt thereof, is used to prevent or treat a haploinsufficiency disease, disorder, or condition. A haploinsufficiency disease, disorder, or condition may refer to a monogenic disease in which an allele of a gene has a loss-of-function lesion, e.g., a total loss of function lesion. In an embodiment, the loss-of-function lesion is present in an autosomal dominant inheritance pattern or is derived from a sporadic event. In an embodiment, the reduction of gene product function due to the altered allele drives the disease phenotype despite the remaining functional allele (i.e. said disease is haploinsufficient with regard to the gene in question). In an embodiment, a compound of Formula (I), (II), (III), or (IV) increases expression of the haploinsufficient gene locus. In an embodiment, a compound of Formula (I), (II), (III), or (IV) increases one or both alleles at the haploinsufficient gene locus. Exemplary haploinsufficiency diseases, disorders, and conditions include Robinow syndrome, cardiomyopathy, cerebellar ataxia, pheochromocytoma, Charcot-Marie-Tooth disease, neuropathy, Takenouchi-Kosaki syndrome, Coffin-Siris syndrome 2, chromosome 1p35 deletion syndrome, spinocerebellar ataxia 47, deafness, seizures, dystonia 9, GLUT1 deficiency syndrome 1, GLUT1 deficiency syndrome 2, stomatin-deficient cryohydrocytosis, basal cell carcinoma, basal cell nevus syndrome, medulloblastoma, somatic, brain malformations, macular degeneration, cone-rod dystrophy, Dejerine-Sottas disease, hypomyelinating neuropathy, Roussy-Levy syndrome, glaucoma, autoimmune lymphoproliferative syndrome, pituitary hormone deficiency, epileptic encephalopathy, early infantile, popliteal pterygium syndrome, van der Woude syndrome, Loeys-Dietz syndrome, Skraban-Deardorff syndrome, erythrocytosis, megalencephaly-polymicrogyria-polydactyly-hydrocephalus syndrome, mental retardation, CINCA syndrome, familial cold inflammatory syndrome 1, keratoendothelitis fugax hereditaria, Muckle-Wells syndrome, Feingold syndrome 1, Acute myeloid leukemia, Heyn-Sproul-Jackson syndrome, Tatton-Brown-Rahman syndrome, Shashi-Pena syndrome, Spastic paraplegia, autosomal dominant, macrophthalmia, colobomatous, with microcornea, holoprosencephaly, schizencephaly, endometrial cancer, familial, colorectal cancer, hereditary nonpolyposis, intellectual developmental disorder with dysmorphic facies and behavioral abnormalities, ovarian hyperstimulation syndrome, schizophrenia, Dias-Logan syndrome, premature ovarian failure, dystonia, dopa-responsive, due to sepiapterin reductase deficiency, Beck-Fahrner syndrome, chromosome 2p12-p11.2 deletion syndrome, neuronopathy, spastic paraplegia, familial adult myoclonic, colorectal cancer, hypothyroidism, Culler-Jones syndrome, holoprosencephaly, myelokathexis, WHIM syndrome, Mowat-Wilson syndrome, mental retardation, an intellectual developmental disorder, autism spectrum disorder, epilepsy, epileptic encephalopathy, Dravet syndrome, migraines, a mental retardation disorder (e.g., a disorder caused by a SETD5 gene mutation, e.g., intellectual disability-facial dysmorphism syndrome, autism spectrum disorder), a disorder caused by a GATA2 mutation (e.g., GATA2 deficiency; GATA2 haploinsufficiency; Emberger syndrome; monocytopenia and Mycobacterium avium complex/dendritic cell, monocyte, B and NK lymphocyte deficiency; familial myelodysplastic syndrome; acute myeloid leukemia; chronic myelomonocytic leukemia), and febrile seizures.

In certain embodiments, the disease, disorder, or condition is an autosomal recessive disease, e.g., with residual function. In certain embodiments, the compound of Formula (I), (II), (III), or (IV), or a pharmaceutically acceptable salt thereof, or compositions comprising such compound or pharmaceutically acceptable salt thereof, is used to prevent or treat an autosomal recessive disease, disorder, or condition. An autosomal recessive disease with residual function may refer to a monogenic disease with either homozygous recessive or compound heterozygous heritability. These diseases may also be characterized by insufficient gene product activity (e.g., a level of gene product greater than 0%). In an embodiment, a compound of Formula (I), (II), (III), or (IV) may increase the expression of a target (e.g., a gene) related to an autosomal recessive disease with residual function. Exemplary autosomal recessive diseases with residual function include Friedreich's ataxia, Stargardt disease, Usher syndrome, chlorioderma, fragile X syndrome, achromatopsia 3, Hurler syndrome, hemophilia B, alpha-1-antitrypsin deficiency, Gaucher disease, X-linked retinoschisis, Wiskott-Aldrich syndrome, mucopolysaccharidosis (Sanfilippo B), DDC deficiency, epidermolysis bullosa dystrophica, Fabry disease, metachromatic leukodystrophy, and odontochondrodysplasia.

In certain embodiments, the disease, disorder, or condition is an autosomal dominant disease. In certain embodiments, the compound of Formula (I), (II), (III), or (IV), or a pharmaceutically acceptable salt thereof, or compositions comprising such compound or pharmaceutically acceptable salt thereof, is used to prevent or treat an autosomal dominant disease, disorder, or condition. An autosomal dominant disease may refer to a monogenic disease in which the mutated gene is a dominant gene. These diseases may also be characterized by insufficient gene product activity (e.g., a level of gene product greater than 0%). In an embodiment, a compound of Formula (I), (II), (III), or (IV) may increase the expression of a target (e.g., a gene) related to an autosomal dominant disease. Exemplary autosomal dominant diseases include Huntington's disease, achondroplasia, antithrombin III deficiency, Gilbert's disease, Ehlers-Danlos syndrome, hereditary hemorrhagic telangiectasia, intestinal polyposis, hereditary elliptosis, hereditary spherocytosis, marble bone disease, Marfan's syndrome, protein C deficiency, Treacher Collins syndrome, Von Willebrand's disease, tuberous sclerosis, osteogenesis imperfecta, polycystic kidney disease, neurofibromatosis, and idiopathic hypoparathyroidism.

In certain embodiments, the disease, disorder, or condition is a paralogue activation disorder. In certain embodiments, the compound of Formula (I), (II), (III), or (IV), or a pharmaceutically acceptable salt thereof, or compositions comprising such compound or pharmaceutically acceptable salt thereof, is used to prevent or treat a paralogue activation disease, disorder, or condition. A paralogue activation disorder may comprise a homozygous mutation of genetic locus leading to loss-of-function for the gene product. In these disorders, there may exist a separate genetic locus encoding a protein with overlapping function (e.g. developmental paralogue), which is otherwise not expressed sufficiently to compensate for the mutated gene. In an embodiment, a compound of Formula (I), (II), (III), or (IV) activates a gene connected with a paralogue activation disorder (e.g., a paralogue gene).

The cell described herein may be an abnormal cell. The cell may be in vitro or in vivo. In certain embodiments, the cell is a proliferative cell. In certain embodiments, the cell is a cancer cell. In certain embodiments, the cell is a non-proliferative cell. In certain embodiments, the cell is a blood cell. In certain embodiments, the cell is a lymphocyte. In certain embodiments, the cell is a benign neoplastic cell. In certain embodiments, the cell is an endothelial cell. In certain embodiments, the cell is an immune cell. In certain embodiments, the cell is a neuronal cell. In certain embodiments, the cell is a glial cell. In certain embodiments, the cell is a brain cell. In certain embodiments, the cell is a fibroblast. In certain embodiment, the cell is a primary cell, e.g., a cell isolated from a subject (e.g., a human subject).

In certain embodiments, the methods described herein comprise the additional step of administering one or more additional pharmaceutical agents in combination with the compound of Formula (I), (II), (III), or (IV), a pharmaceutically acceptable salt thereof, or compositions comprising such compound or pharmaceutically acceptable salt thereof. Such additional pharmaceutical agents include, but are not limited to, anti-proliferative agents, anti-cancer agents, anti-diabetic agents, anti-inflammatory agents, immunosuppressant agents, and a pain-relieving agent. The additional pharmaceutical agent(s) may synergistically augment the modulation of splicing induced by the inventive compounds or compositions of this disclosure in the biological sample or subject. Thus, the combination of the inventive compounds or compositions and the additional pharmaceutical agent(s) may be useful in treating, for example, a cancer or other disease, disorder, or condition resistant to a treatment using the additional pharmaceutical agent(s) without the inventive compounds or compositions.

### EXAMPLES

In order that the invention described herein may be more fully understood, the following examples are set forth. The examples described in this application are offered to illustrate the compounds, pharmaceutical compositions, and methods provided herein and are not to be construed in any way as limiting their scope.

The compounds provided herein can be prepared from readily available starting materials using modifications to the specific synthesis protocols set forth below that would be well known to those of skill in the art. It will be appreciated that where typical or preferred process conditions (i.e., reaction temperatures, times, mole ratios of reactants, solvents, pressures, etc.) are given, other process conditions can also be used unless otherwise stated. Optimum reaction conditions may vary with the particular reactants or solvents used, but such conditions can be determined by those skilled in the art by routine optimization procedures.

Additionally, as will be apparent to those skilled in the art, conventional protecting groups may be necessary to prevent certain functional groups from undergoing undesired reactions. The choice of a suitable protecting group for a particular functional group as well as suitable conditions for protection and deprotection are well known in the art. For example, numerous protecting groups, and their introduction and removal, are described in Greene et al., Protecting Groups in Organic Synthesis, Second Edition, Wiley, New York, 1991, and references cited therein.

Reactions can be purified or analyzed according to any suitable method known in the art. For example, product formation can be monitored by spectroscopic means, such as nuclear magnetic resonance (NMR) spectroscopy (e.g., ¹H or ¹³C), infrared (IR) spectroscopy, spectrophotometry (e.g., UV-visible), mass spectrometry (MS), or by chromatographic methods such as high performance liquid chromatography (HPLC) or thin layer chromatography (TLC). In some embodiments, absolute stereochemistry of chiral compounds provided herein is arbitrarily assigned.

Proton NMR: ¹H NMR spectra were recorded in CDCl₃ solution in 5-mm o.d. tubes (Wildmad) at 24° C. and were collected on a BRUKER AVANCE NEO 400 at 400 MHz for ¹H. The chemical shifts (6) are reported relative to tetramethylsilane (TMS=0.00 ppm) and expressed in ppm.

LC/MS: Liquid chromatography-mass spectrometry (LC/MS) was performed on Shimadzu-2020EV using column: Shim-pack XR-ODS (C18, Ø4.6×50 mm, 3 μm, 120 Å, 40° C.) operating in ESI(+) ionization mode; flow rate=1.2 mL/min. Mobile phase=0.05% TFA in water or CH₃CN; or on Shimadzu-2020EV using column: Poroshell HPH-C18 (C18, Ø4.6×50 mm, 3 μm, 120 Å, 40° C.) operating in ESI(+) ionization mode; flow rate=1.2 mL/min. Mobile phase A: Water/5 mM NH₄HCO₃, Mobile phase B: CH₃CN.)

Analytical chiral HPLC: Analytical chiral HPLC was performed on a Agilent 1260 using column: CHIRALPAK IG-3, CHIRALPAK IC-3 or CHIRALPAK OJ-3, with flow rate=1.2 mL/min. Mobile phase=MTBE(DEA): EtOHL:=50:50).

Preparative HPLC purification: prep-HPLC purification was performed on a Waters-2545 or Shimadzu, using one of the following conditions:
Condition 1: Column: X-Select CSH C18 OBD (130 Å, 5 μm, 30 mm×150 mm); Mobile phase A: water (10 mmol/L NH₄HCO₃); Mobile phase B: acetonitrile; Gradient 1: 5% B up to 85% B in 8 min; Gradient 2: 10% B to 40% B in 8 min; Gradient 3: 5% B up to 55% B in 8 min; Gradient 4: 5% B up to 40% B in 8 min.
Condition 2: Column: XBridge Prep OBD C18 (30×150 mm, 5 μm); Mobile phase A: water (10 mmol/L NH₄HCO₃); Mobile phase B: acetonitrile; Gradient 1: 5% B up to 65% B in 8 min; Gradient 2: 5% B to 48% B in 8 min; Gradient 3: 10% B to 55% B in 8 min; Gradient 3: 5% B to 55% B in 8 min; Gradient 4: 5% B to 45% B in 8 min; Gradient 5: 5% B to 50% B in 8 min; Gradient 6: 25% B to 65% B in 8 min.
Condition 3: Column: XBridge Prep C18 OBD (Sum, 19 mm×150 mm).
Condition 4: Column: YMC-Actus Triart C18 (30×150 mm, 5 μm); Mobile phase A: water (10 mM ammonium formate); Mobile phase B: acetonitrile; Gradient 1: 15% B to 95% B in 8 min.
Condition 5: Column: YMC-Actus Triart C18 (30×150 mm, 5 μm); Mobile phase A: water (10 mmol/L NH₄HCO₃); Mobile phase B: acetonitrile; Flow rate: 60 mL/min; Gradient 1: 55% B to 77% B in 8 min; Gradient 2: 10% B to 34% B in 10 min; Gradient 3: 10% B to 75% B in 8 min; Gradient 4: 45% B to 85% B in 8 min; Gradient 5: 25% B to 85% B in 8 min; Gradient 6: 5% B up to 35% B in 8 min; Gradient 7: 5% B to 75% B in 8 min; Gradient 8: 25% B to 61% B in 8 min; Gradient 9: 5% B to 80% B in 8 min; Gradient 10: 20% B to 47% B in 8 min.
Condition 6: Column: Column: XBridge Prep OBD C18 (30×150 mm, 5 μm); Mobile Phase A: water (0.05% HCl), Mobile Phase B: ACN; Flow rate:60 mL/min; Gradient 1:5% B to 45% B in 8 min.
Condition 7: Column: XSelect CSH OBD Column (300×150 mm, 5 μm, n); Mobile phase A: water (0.05% HCl); Mobile phase B: CAN; Gradient 1: 3% Phase B up to 40% Phase B in 8 min.
Condition 8: Column: YMC-Actus Triart C18, 30*150 mm, 5 μm; Mobile Phase A: water (0.05% HCl), Mobile Phase B: acetonitrile; Flow rate: 60 mL/min; Gradient 1: 5% B to 40% B in 8 min.

Flash Preparative HPLC purification: Flash-Prep-HPLC purification was performed using one of the following conditions:
Condition 1: Column: C18 silica gel; Mobile phase A: water (10 mmol/L NH₄HCO₃); Mobile phase B: acetonitrile; Gradient 1: 40% B up to 80%.
Condition 2: IntelFlash-1, Column: silica gel; Mobile phase A: water; Mobile phase B: acetonitrile; Gradient 1: 10% B up to 90% B in 30 min; Gradient 2: 55% B up to 77% B in 8 min; Detector: UV 254 nm.

Preparative chiral HPLC: purification by chiral HPLC was performed on a Gilson-GX 281 using column: CHIRALPAK IG-3, CHIRALPAK IC-3 or CHIRALPAK OJ-3.

### General Schemes

Compounds of the present disclosure may be prepared using a synthetic protocol illustrated below in Schemes A, B, C, D, E, F, G, and H.

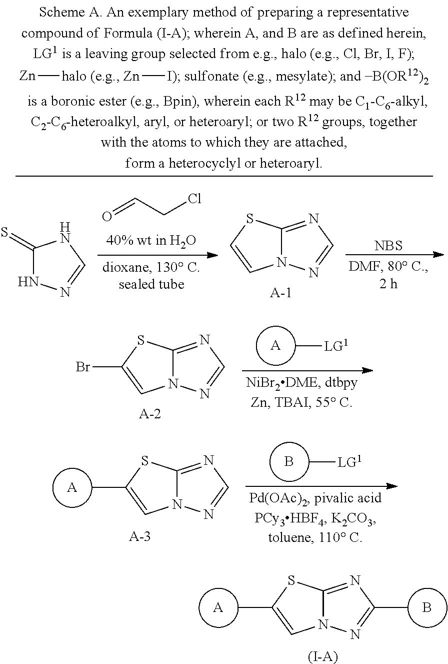

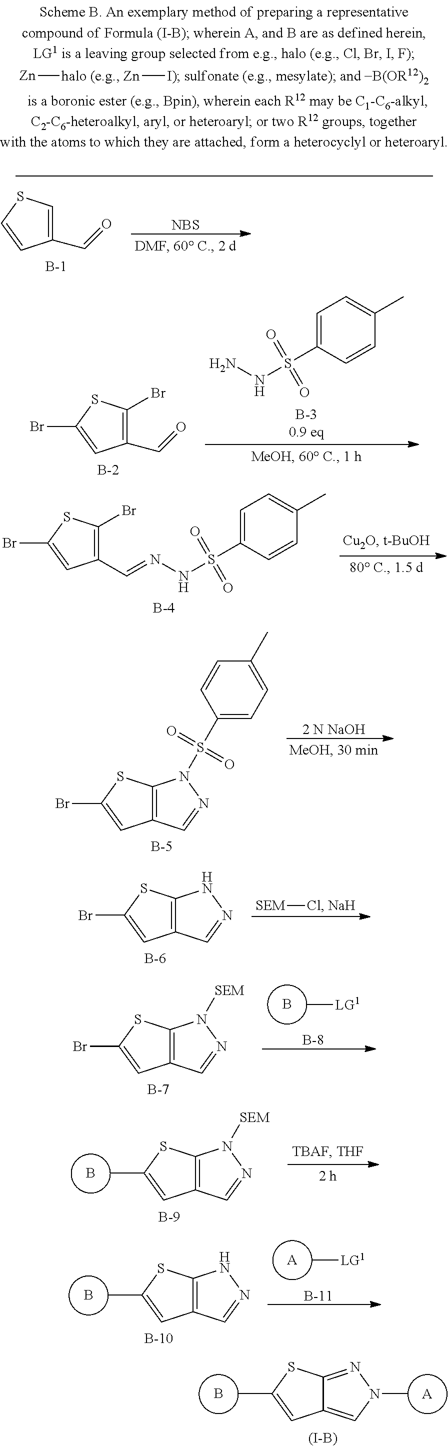

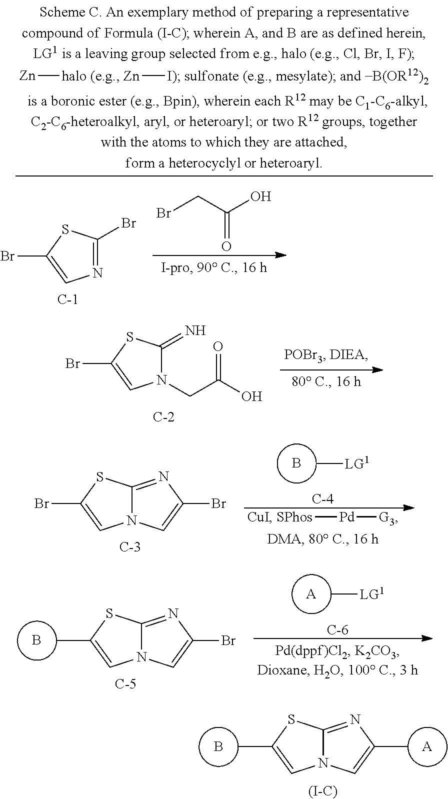

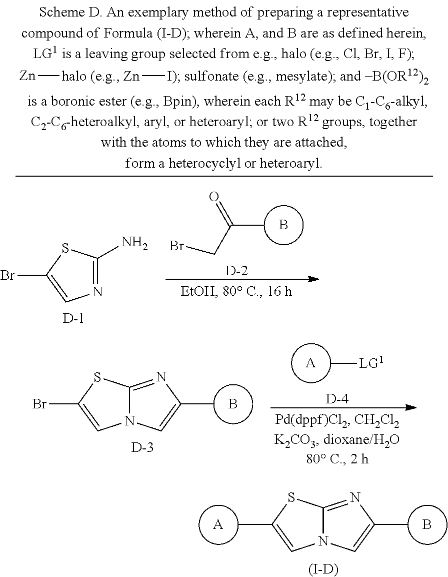

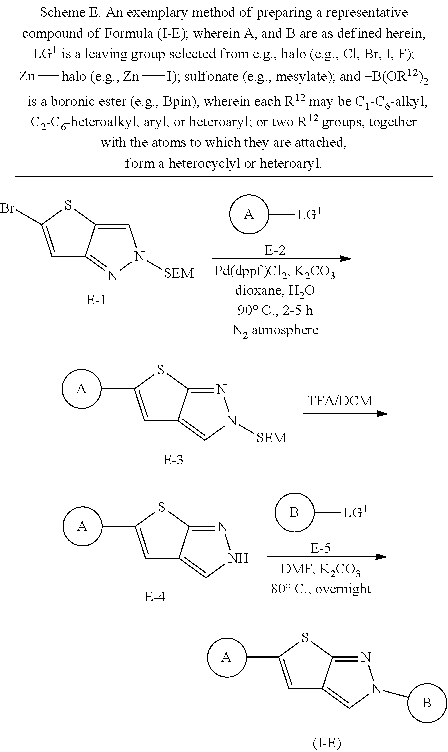

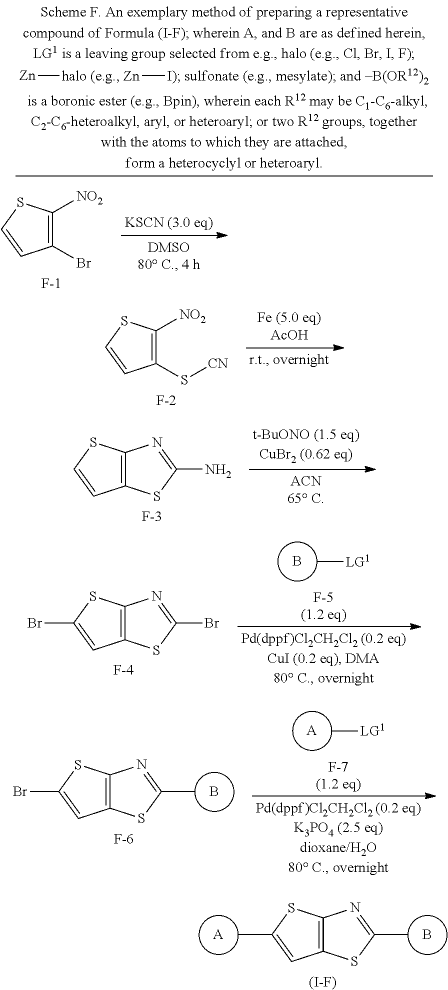

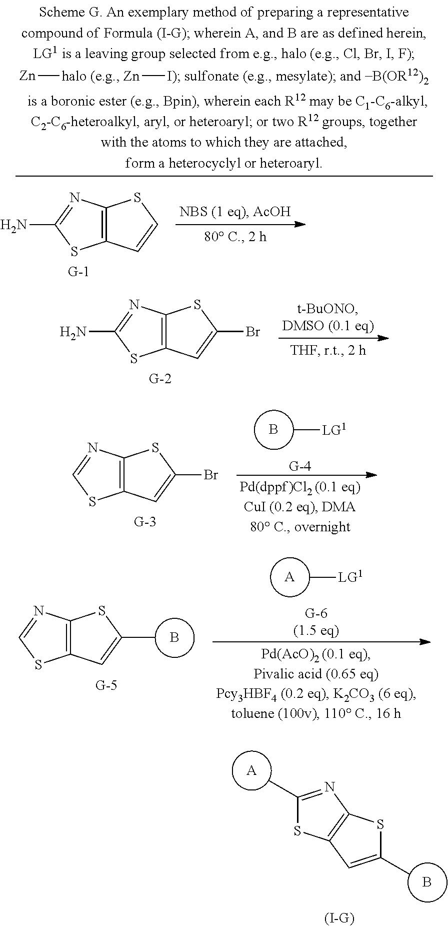

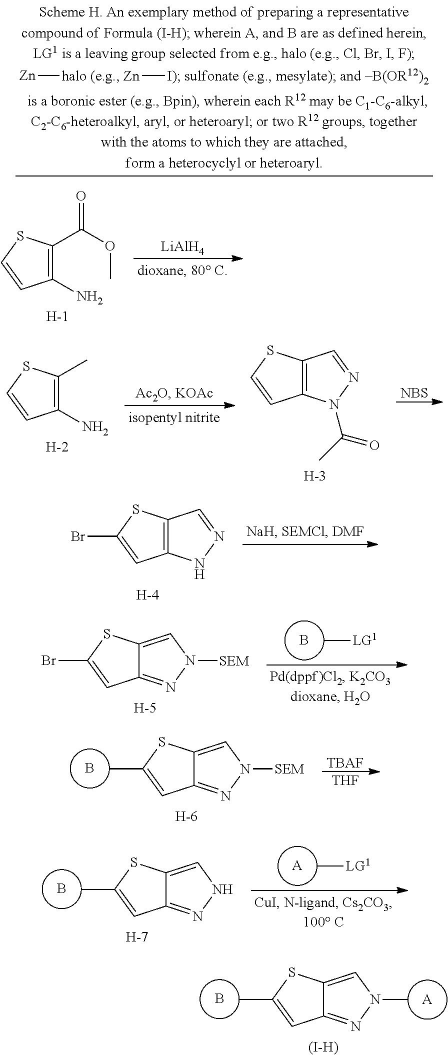

Exemplary methods of preparing a compound of Formula (I) are provided in Schemes A-H. Coupling of Ring A or Ring B to the core may be carried out with a palladium catalyst, such as Pd₂(dba)₃, [1,1′-bis(di-tert-butylphosphino)ferrocene]dichloropalladium(II) (Pd(dtbpf)Cl₂) or chloro(2-dicyclohexylphosphino-2′,4′,6′-triisopropyl-1,1′-biphenyl)[2-(2′-amino-1,1′-biphenyl)]palladium(II) (XPhos-Pd-G2). Coupling reactions may be conducted in DMA, DMF, toluene, dioxane, water, or a similar solvent or mixtures of solvents, at 80° C. or a temperature sufficient to provide the compound of Formula (I), for example, 80° C., 90° C., 100° C., 110° C., or 120° C. The reaction may be conducted in a microwave reactor. Compounds of Formula (I) may be purified using standard techniques and characterized using any method known in the art, such as nuclear magnetic resonance spectroscopy (NMR) or mass spectrometry (MS).

### Example 1: Synthesis of Compound 102

### Synthesis of Intermediate B2

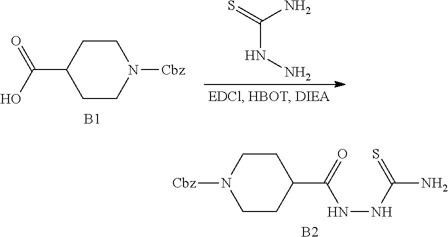

1-Ethyl-3-(3-dimethylaminopropyl)carbodiimide hydrochloride (7.3 g, 38 mmol), hydroxybenzotriazole (5.1 g, 38 mmol) and diisopropylethylamine (14.7 g, 114 mmol) were added to a solution of 1-[(benzyloxy)carbonyl]piperidine-4-carboxylic acid (B1; 10 g, 38 mmol) and thiosemicarbazide (3.46 g, 38 mmol) in dimethylformamide (100 mL), and the mixture was stirred for 3 h at room temperature. The resulting mixture was then diluted with water and acidified with 1M HCl to achieve a pH of 5-6. The precipitated solids were collected by filtration and washed with water, to afford benzyl 4-(carbamothioylaminocarbamoyl)piperidine-1-carboxylate (B2; 2 g) as a solid. LCMS (ES, m z): 337 [M+H]⁺.

### Synthesis of Intermediate B3

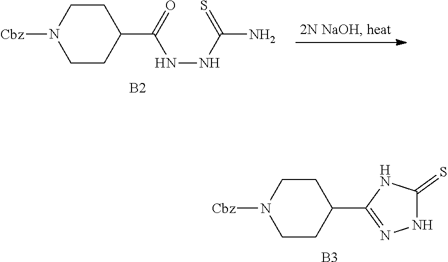

A mixture of benzyl 4-(carbamothioylaminocarbamoyl)piperidine-1-carboxylate (B2; 12 g, 36 mmol) and 1M sodium hydroxide (100 mL) was stirred for 1 h at 50° C. The residue was acidified with 1M HCl to achieve a pH of 5 and extracted with dichloromethane (3×300 mL). The combined organic layers were washed with brine (100 mL), dried over anhydrous sodium sulfate, filtered, and concentrated under reduced pressure to give a residue. The residue was purified by silica gel column chromatography, eluting with dichloromethane/methanol (50:1), to afford benzyl 4-(5-sulfanylidene-1,4-dihydro-1,2,4-triazol-3-yl)piperidine-1-carboxylate (B3; 7 g) as a solid. LCMS (ES, m/z): 319 [M+H]⁺.

### Synthesis of Intermediate B4

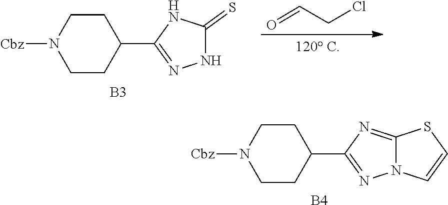

Chloroacetaldehyde (3.45 g, 44 mmol) was added dropwise to a solution of benzyl 4-(5-sulfanylidene-1,4-dihydro-1,2,4-triazol-3-yl)piperidine-1-carboxylate (B3; 7 g, 22 mmol) in 1,4-dioxane (60 mL) in a pressure tank reactor, and the resulting mixture was stirred for 4 h at 120° C. The mixture was then concentrated under reduced pressure, and purified by silica gel column chromatography eluting with dichloromethane/methanol (40:1), to afford benzyl 4-[[1,2,4]triazolo[3,2-b][1,3]thiazol-2-yl]piperidine-1-carboxylate (B4; 1.3 g) as a solid. LCMS (ES, m/z): 343 [M+H]⁺.

### Synthesis of Intermediate B5

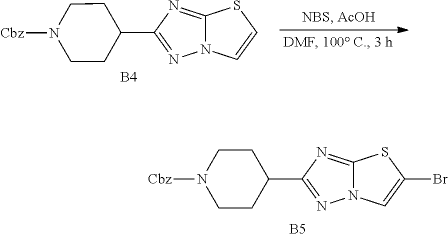

N-Bromosuccinimide (623 mg, 3.5 mmol) and acetic acid (28 mg, 0.4 mmol) were added dropwise to a solution of benzyl 4-[[1,2,4]triazolo[3,2-b][1,3]thiazol-2-yl]piperidine-1-carboxylate (B4; 800 mg, 2.3 mmol) in dimethylformamide at room temperature, and the resulting mixture was stirred for 16 h at 100° C. under a nitrogen atmosphere. The reaction was quenched with water at room temperature, and the resulting mixture was extracted with ethyl acetate (100 mL). The combined organic layers were washed with brine (4×20 mL), dried over anhydrous sodium sulfate, filtered, and concentrated under reduced pressure. The residue was purified by silica gel column chromatography, eluting with dichloromethane/ethyl acetate (1:1) to afford benzyl 4-[5-bromo-[1,2,4]triazolo[3,2-b][1,3]thiazol-2-yl]piperidine-1-carboxylate (B5; 370 mg) as a solid. LCMS (ES, m/z): 422[M+2]⁺.

### Synthesis of Intermediate B7

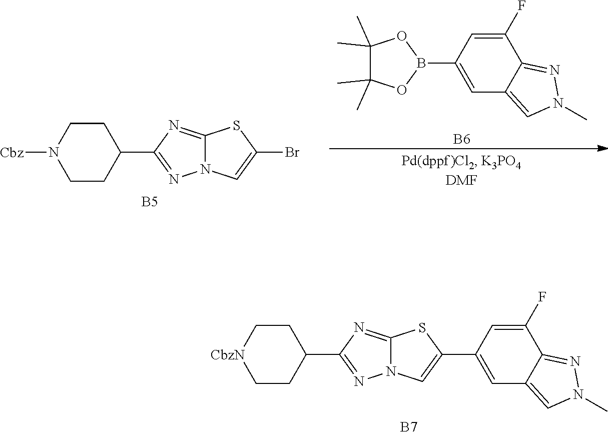

Tripotassium phosphate (75 mg, 0.3 mmol) and Pd(dppf)Cl₂ (17 mg, 0.02 mmol) were added to a solution of benzyl 4-[5-bromo-[1,2,4]triazolo[3,2-b][1,3]thiazol-2-yl]piperidine-1-carboxylate (B5; 50 mg, 0.12 mmol) and 7-fluoro-2-methyl-5-(4,4,5,5-tetramethyl-1,3,2-dioxaborolan-2-yl)indazole (B6; 39 mg, 0.14 mmol) in dimethylformamide (4 mL) and water (1 mL), and the resulting mixture was stirred for 3 h at 80° C. under a nitrogen atmosphere. The mixture was concentrated under reduced pressure and extracted with ethyl acetate (2×30 mL). The combined organic layers were washed with brine (10 mL), dried over anhydrous sodium sulfate, filtered, and concentrated under reduced pressure. The residue was purified by silica gel column chromatography, eluting with dichloromethane/methanol (30:1), to afford tert-butyl 4-[5-(7-fluoro-2-methylindazol-5-yl)-[1,2,4]triazolo[3,2-b][1,3]thiazol-2-yl]piperidine-1-carboxylate (B7; 35 mg) as a solid. LCMS (ES, m/z): 491 [M+H]⁺.

### Synthesis of Compound 102

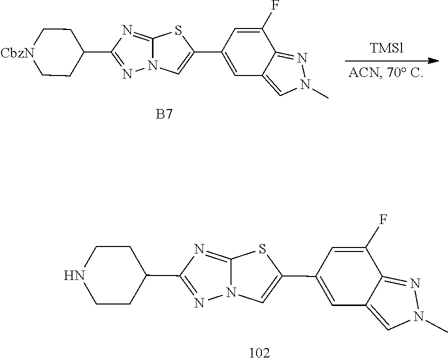

Iodotrimethylsilane (21 mg, 0.11 mmol) was added dropwise to a solution of benzyl 4-[5-(7-fluoro-2-methylindazol-5-yl)-[1,2,4]triazolo[3,2-b][1,3]thiazol-2-yl]piperidine-1-carboxylate (B7; 35 mg, 0.07 mmol) in acetonitrile (2 mL) at room temperature, and the resulting mixture was stirred for 15 min at 70° C. The reaction was quenched with methanol at room temperature and concentrated under vacuum. The residue was purified by silica gel column chromatography, eluting with dichloromethane/methanol (30:1), to afford 7-fluoro-2-methyl-5-[2-(piperidin-4-yl)-[1,2,4]triazolo[3,2-b][1,3]thiazol-5-yl]indazole (Compound 108; 10.1 mg) as a solid. LCMS (ES, m/z): 357 [M+H]⁺. ¹H NMR (400 MHz, DMSO-d6) δ 8.86 (s, 1H), 8.56 (d, J=2.8 Hz, 1H), 7.83 (d, J=1.4 Hz, 1H), 7.57 (dd, J=12.7, 1.5 Hz, 1H), 4.23 (s, 3H), 3.02 (dt, J=12.3, 3.5 Hz, 2H), 2.88 (tt, J=11.4, 3.8 Hz, 1H), 2.62 (td, J=12.0, 2.6 Hz, 2H), 1.92 (dd, J=13.5, 3.4 Hz, 2H), 1.65 (qd, J=11.8, 3.8 Hz, 2H).

### Example 2: Synthesis of Compound 100

### Synthesis of Intermediate B9

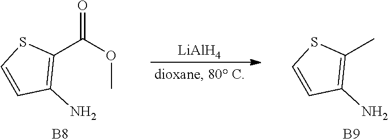

To a stirred solution of B8 (30.00 g, 190.852 mmol, 1.00 equiv) in dioxane (300.00 mL) was added LiAlH₄ (14.44 g, 381.704 mmol, 2.00 equiv) at room temperature. The mixture was stirred 2 h at 80° C. The reaction was quenched by the addition of 14 mL water, 14 mL 15% NaOH, 42 mL water, and the resulting mixture was filtered and the filter cake was washed with EA. The combined organic layers were dried over anhydrous Na₂SO₄, was concentrated under reduced pressure. The crude product was used in the next step directly without further purification.

### Synthesis of Intermediate B11

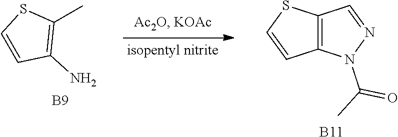

To a stirred solution of B9 (20.00 g, 176.71 mmol, 1.00 equiv) in AcOH (200.00 mL, 3490.308 mmol) was added AcOK (17.34 g, 176.71 mmol, 1.00 equiv) and Ac₂O (27.06 g, 265.064 mmol, 1.50 equiv) dropwise at 0° C. The temperature was increased to 80° C. and isopentyl nitrite (31.06 g, 265.064 mmol, 1.50 equiv) was added. The mixture was stirred 15 h at 100° C., then the resulting mixture was concentrated under vacuum. The mixture was basified to pH 8 with NaHCO₃, and the aqueous layer was extracted with EA. The residue was purified by silica gel column chromatography and the product was eluted with PE:EA (4:1) to afford 1-[thieno[3,2-c]pyrazol-1-yl]ethanone (B11, 13 g, 44.55%) as an oil.

### Synthesis of Intermediate B12

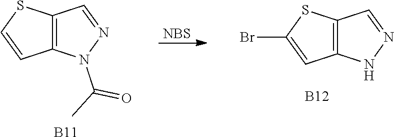

To a stirred solution of B11 (8.00 g, 48.134 mmol, 1.00 equiv) in AcOH (80.00 mL, 1396.124 mmol) was added NBS (12.86 g, 72.202 mmol, 1.50 equiv) at 70° C. The mixture was stirred 15 h at 70° C., and the solution was concentrated under vacuum. The residue was basified to pH 8 with NaHCO₃ and the aqueous layer was extracted with EA. The residue was purified by silica gel column chromatography and the product eluted with PE:EA (4:1) to afford 5-bromo-1H-thieno[3,2-c]pyrazole (B12, 2.90 g, 29.65%) as an oil. LCMS (ES, m/z): 203 [M+H]⁺

### Synthesis of Intermediate B13

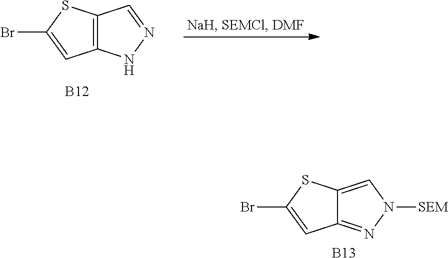

To a stirred solution of B12 (2.90 g, 14.281 mmol, 1.00 equiv) in DMF (30.00 mL) was added NaH (514.08 mg, 21.422 mmol, 1.50 equiv) at 0° C. The mixture was stirred for 30 min, then [2-(chloromethoxy)ethyl]trimethylsilane (2.857 g, 17.138 mmol, 1.2 equiv) was added. The mixture was stirred 1 h at room temperature, then the reaction was quenched with water. The aqueous layer was extracted with EA and the residue was purified by silica gel column chromatography with PE:EA (4:1) to afford 5-bromo-1-[[2-(trimethylsilyl)ethoxy]methyl]thieno[3,2-c]pyrazole (B13, 2.80 g, 59.57%) as an oil. LCMS (ES, m/z): 333 [M+H]⁺

### Synthesis of Intermediate B14

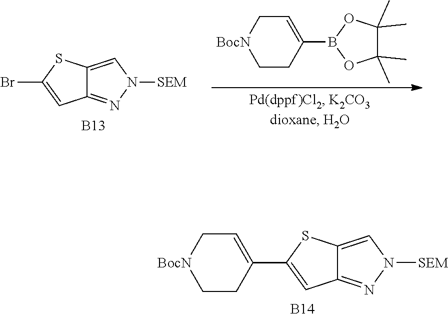

To a solution of B13 (1.80 g, 5.400 mmol, 1.00 equiv) and tert-butyl 4-(4,4,5,5-tetramethyl-1,3,2-dioxaborolan-2-yl)-3,6-dihydro-2H-pyridine-1-carboxylate (1.67 g, 5.401 mmol, 1.00 equiv) in dioxane (20.00 mL) and water (4.00 mL) were added Pd(dppf)Cl₂ (395.13 mg, 0.540 mmol, 0.10 equiv) and K₂CO₃ (2.24 g, 16.201 mmol, 3.00 equiv). After stirring for 2 h at 80° C. under a nitrogen atmosphere, the resulting mixture was concentrated under reduced pressure. The residue was purified by silica gel column chromatography and eluted with PE:EA (2:1) to afford tert-butyl 4-(2-[[2-(trimethylsilyl)ethoxy]methyl]thieno[3,2-c]pyrazol-5-yl)-3,6-dihydro-2H-pyridine-1-carboxylate (B14, 1.6 g, 68.01%) as a solid. LCMS (ES, m/z): 436 [M+H]⁺

### Synthesis of Intermediate BJ5

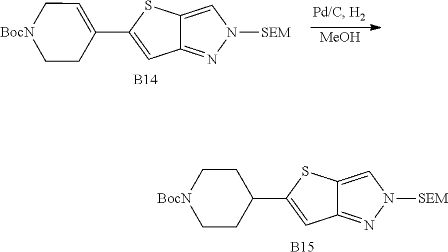

To a stirred solution of B14 (1.60 g, 3.673 mmol, 1.00 equiv) in MeOH (20.00 mL, 493.978 mmol, 134.50 equiv) was added Pd/C (160.24 mg, 1.506 mmol, 0.41 equiv) at room temperature under H₂ atmosphere. The mixture was stirred 15 h at room temperature. The resulting mixture was filtered and the filter cake was washed with MeOH. The filtrate was concentrated under reduced pressure to afford tert-butyl 4-(2-[[2-(trimethylsilyl)ethoxy]methyl]thieno[3,2-c]pyrazol-5-yl)piperidine-1-carboxylate (B15, 1.3 g, 80.88%) as an oil.

### Synthesis of Intermediate B16

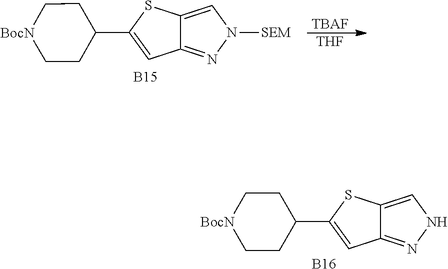

To a stirred solution of B15 (1.60 g, 3.656 mmol, 1.00 equiv) in THE (20.00 mL, 277.383 m mol) was added TBAF (1.91 g, 7.311 mmol, 2.00 equiv) at room temperature. The mixture was stirred 2 h at 80° C., and the solution was extracted with ethyl aceteate (EA) and washed with water. The combined organic layers were dried over anhydrous Na₂SO₄ and concentrated under reduced pressure to afford tert-butyl 4-[2H-thieno[3,2-c]pyrazol-5-yl]piperidine-1-carboxylate (B16, 380 mg, 33.81%) as an oil.

### Synthesis of Intermediate B17

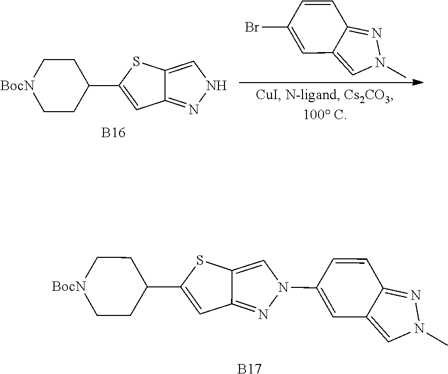

To a stirred solution of B16 (130.00 mg, 0.423 mmol, 1.00 equiv) and 5-bromo-2-methylindazole (89.26 mg, 0.423 mmol, 1.00 equiv) in DMF (4.00 mL) was added (1S,2S)-(+)-1,2-diaminocyclohexane (24.14 mg, 0.211 mmol, 0.50 equiv) and CuI (40.27 mg, 0.211 mmol, 0.50 equiv) at 100° C. under N₂ atmosphere. The mixture was stirred 15 h at 100° C., then the crude product was purified by prep-HPLC to afford tert-butyl 4-[2-(2-methylindazol-5-yl)thieno[3,2-c]pyrazol-5-yl]piperidine-1-carboxylate (B17, 40 mg, 21.62%) as a solid, and tert-butyl 4-[1-(2-methylindazol-5-yl)thieno[3,2-c]pyrazol-5-yl]piperidine-1-carboxylate (B17-A, 30 mg, 16.21%) as a solid. LCMS (ES, m/z): 438 [M+H]⁺

### Synthesis of Compound 100

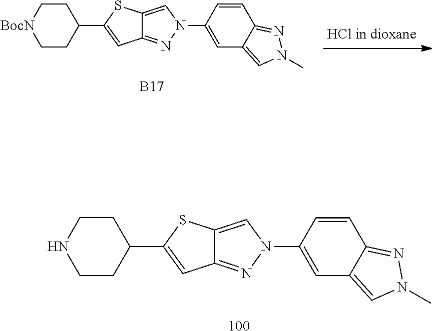

To a stirred solution of B17 (30.00 mg, 1 equiv) in MeOH (4.00 mL) was added HCl(gas)in 1,4-dioxane (1.00 mL) at room temperature. The mixture was stirred 1 h at room temperature, then the crude product was purified by prep-HPLC to afford 2-methyl-5-[5-(piperidin-4-yl)thieno[3,2-c]pyrazol-2-yl]indazole (Compound 100, 9.8 mg, 42.36%) as a solid. LCMS (ES, m/z): 338 [M+H]+. ¹H NMR (400 MHz, DMSO-d6) δ 8.66 (s, 1H), 8.44 (s, 1H), 8.12 (d, J=2.1 Hz, 1H), 7.83 (dd, J=9.2, 2.2 Hz, 1H), 7.74 (d, J=9.3 Hz, 1H), 6.98 (s, 1H), 4.20 (s, 3H), 3.03 (d, J=12.0 Hz, 2H), 2.90 (td, J=11.6, 6.0 Hz, 1H), 2.60 (t, J=11.8 Hz, 2H), 1.96-1.88 (m, 2H), 1.56 (qd, J=12.2, 3.9 Hz, 2H), 1.22 (s, 1H).

### Example 3: Synthesis of Compound 111

### Synthesis of Intermediate B18

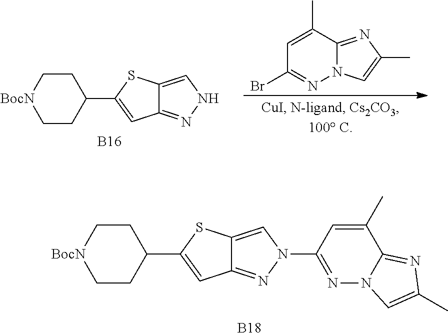

To a stirred solution of B16 (200.00 mg, 0.651 mmol, 1.00 equiv) and 6-bromo-2,8-dimethylimidazo[1,2-b]pyridazine (147.09 mg, 0.651 mmol, 1.00 equiv) in dioxane (5.00 mL) was added CuI (61.95 mg, 0.325 mmol, 0.50 equiv), (1S,2S)—N1,N2-dimethylcyclohexane-1,2-diamine (46.27 mg, 0.325 mmol, 0.50 equiv) and Cs₂CO₃ (635.93 mg, 1.952 mmol, 3.00 equiv) at 100° C. under N₂ atmosphere. The mixture was stirred 15 h at 100° C., then the residue was purified by silica gel column chromatography, eluting with PE:EA (1:1) to afford a mixture comprising product. The mixture was further purified by prep-HPLC with the following conditions to afford tert-butyl 4-(2-[2,8-dimethylimidazo[1,2-b]pyridazin-6-yl]thieno[3,2-c]pyrazol-5-yl)piperidine-1-carboxylate (80 mg) as a solid and tert-butyl 4-(1-[2,8-dimethylimidazo[1,2-b]pyridazin-6-yl]thieno[3,2-c]pyrazol-5-yl)piperidine-1-carboxylate (B18, 60 mg) as a solid. LCMS (ES, m/z): 453 [M+H]⁺.

### Synthesis of Compound 111

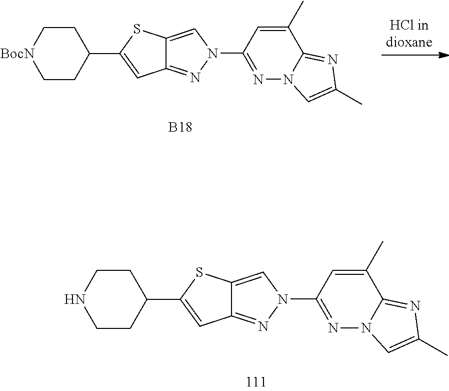

To a stirred solution of B18 (80.00 mg, 1 equiv) in MeOH (5.00 mL) was added HCl (gas) in 1,4-dioxane (2.00 mL) at room temperature. The mixture was stirred 2 h at room temperature, then the mixture was purified by prep-HPLC to afford 4-(2-[2,8-dimethylimidazo[1,2-b]pyridazin-6-yl]thieno[3,2-c]pyrazol-5-yl)piperidine (Compound 111, 6.4 mg, 58.42%) as a solid. LCMS (ES, m/z): 353 [M+H]^{+ 1}H NMR (400 MHz, DMSO-d6) δ 8.13 (s, 1H), 8.08 (s, 1H), 7.69 (s, 1H), 7.56 (s, 1H), 3.02 (q, J=11.8 Hz, 3H), 2.61 (s, 4H), 2.59 (s, 1H), 2.39 (s, 3H), 1.96 (d, J=12.5 Hz, 2H), 1.62 (dd, J=12.3, 3.9 Hz, 1H), 1.56 (dd, J=12.1, 4.0 Hz, 1H).

### Example 4: Synthesis of Compound 121

### Synthesis of Intermediate B21

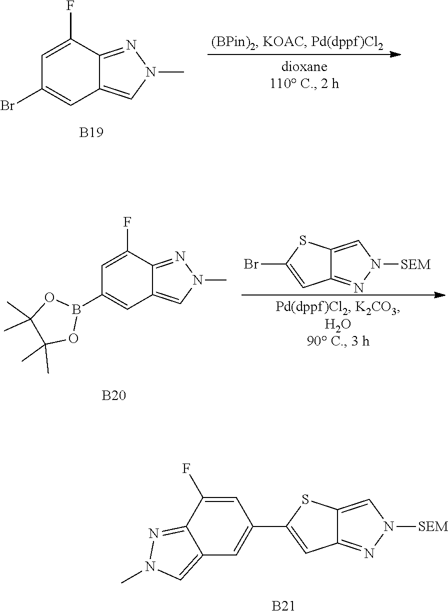

The mixture of B19 (1.00 g, 4.366 mmol, 1.00 equiv), bis(pinacolato)diboron (1.11 g, 4.371 mmol, 1.00 equiv) and KOAc (320.69 mg, 3.268 mmol, 3.0 equiv), Pd(dppf)Cl₂ (0.32 g, 0.437 mmol, 0.10 equiv) in dioxane (20.00 mL). The resulting mixture was stirred for 2 h at 110° C., at which point the desired product was observed by LCMS. To this mixture was added 5-bromo-2-[[2-(trimethylsilyl) ethoxy]methyl]thieno[3,2-c]pyrazole (1.46 g, 4.380 mmol, 1.00 equiv), K₂CO₃ (451.60 mg, 3.268 mmol, 3.0 equiv) and Pd(dppf)Cl₂ (0.32 g, 0.437 mmol, 0.10 equiv), water (4.00 mL). The resulting mixture was stirred for 3 h at 90° C., then concentrated under reduced pressure. The residue was purified by silica gel column chromatography, eluted with hexane/EtOAc (1:1) to afford 7-fluoro-2-methyl-5-(2-[[2-(trimethylsilyl) ethoxy]methyl]thieno[3,2-c]pyrazol-5-yl)indazole (B21, 990 mg,56.33%) as a solid. LCMS (ES, m/z): 403 [M+H]⁺.

### Synthesis of Intermediate B22

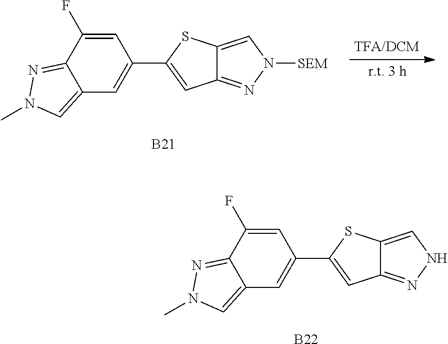

Into a 40 mL round-bottom flask were added B21 (400.00 mg, 0.994 mmol, 1.00 equiv) and TFA (4.00 mL), DCM (4.00 mL) at room temperature. The resulting mixture was stirred for 3 h at room temperature, then concentrated under reduced pressure and extracted with CH₂Cl₂ (3×4 mL). The combined organic layers were washed with brine (2×3 mL), dried over anhydrous Na₂SO₄, filtered, then concentrated under reduced pressure to afford 7-fluoro-2-methyl-5-[2H-thieno[3,2-c]pyrazol-5-yl]indazole (B22, 200 mg, 73.92%) as a solid. LCMS (ES, m/z): 273 [M+H]⁺

### Synthesis of Intermediate B23

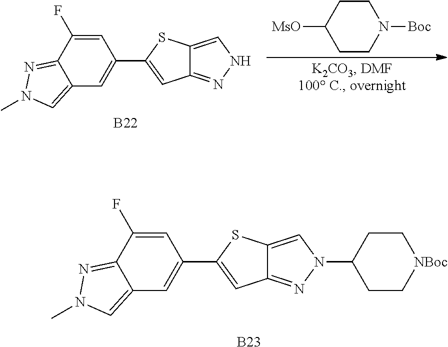

Into a 40 mL round-bottom flask were B22 (200.00 mg, 0.734 mmol, 1.00 equiv) and tert-butyl 4-(methanesulfonyloxy)piperidine-1-carboxylate (205.18 mg, 0.734 mmol, 1 equiv), K₂CO₃(304.53 mg, 2.203 mmol, 3.00 equiv), and DMF (10.00 mL) at room temperature. The resulting mixture was stirred for overnight at 100° C. under nitrogen atmosphere, then the mixture was allowed to cool to room temperature. The crude product (150 mg) was purified by prep-HPLC to afford tert-butyl 4-[5-(7-fluoro-2-methylindazol-5-yl)thieno[3,2-c]pyrazol-1-yl]piperidine-1-carboxylate; tert-butyl-4-[5-(7-fluoro-2-methylindazol-5-yl)thieno[3,2-c]pyrazol-2-yl]piperidine-1-carboxylate (34 mg) as a solid and tert-butyl 4-(5-(7-fluoro-2-methyl-2H-indazol-5-yl)-2H-thieno[3,2-c]pyrazol-2-yl)piperidine-1-carboxylate (40 mg) as a solid. LCMS (ES, m/z): 456 [M+H]⁺

### Synthesis of Compound 121

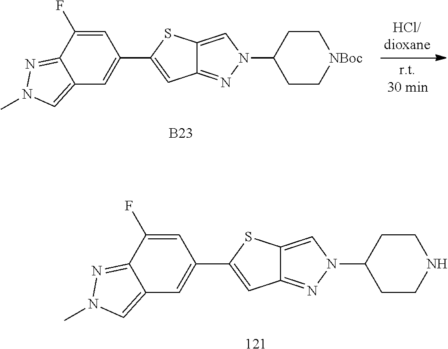

Into a 8 mL round-bottom flask was added B23 (29.00 mg) and HCl(gas) in 1,4-dioxane (6.00 mL) at room temperature. The resulting mixture was stirred for 30 min at room temperature. The crude product (20 mg) was purified by prep-HPLC to afford 7-fluoro-2-methyl-5-[2-(piperidin-4-yl)thieno[3,2-c]pyrazol-5-yl]indazole (Compound 121, 12 mg) as a solid. LCMS (ES, m/z): 356 [M+H]^{+ 1}HNMR (400 MHz, DMSO-d₆, ppm) δ 8.52 (d, J=2.8 Hz, 1H), 8.07 (s, 1H), 7.80 (d, J=1.4 Hz, 1H), 7.62 (s, 1H), 7.55 (dd, J=13.1, 1.4 Hz, 1H), 4.36 (tt, J=11.4, 4.0 Hz, 1H), 4.22 (s, 3H), 4.21 (s, 1H), 3.11-3.03 (m, 2H), 2.63 (td, J=12.3, 2.5 Hz, 2H), 2.49 (s, 1H), 2.06-1.97 (m, 2H), 1.89 (qd, J=12.0, 4.1 Hz, 2H).

### Example 5: Synthesis of Compound 103

### Synthesis of Intermediate B25

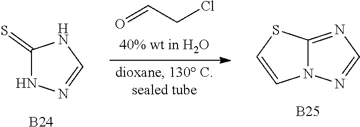

A mixture of 2,4-dihydro-1,2,4-triazole-3-thione (B24; 40 g, 388 mmol) and chloroacetaldehyde (76 g, 388 mmol, 40%) in dioxane (400 mL) was stirred for 16 h at 130° C. in a sealed tube. The mixture was then cooled to 20° C. and concentrated under reduced pressure. The residue was purified by silica gel column chromatography, eluting with dichloromethane/methanol (10:1), and the resulting solution was concentrated under vacuum. The residue was then recrystallized from ethyl acetate/methanol (10:1; 300 mL) to afford [1,2,4]triazolo[3,2-b][1,3]thiazole (B25; 9 g) as a solid. LCMS (ES, m/z): 126 [M+H]⁺.

### Synthesis of Intermediate B26

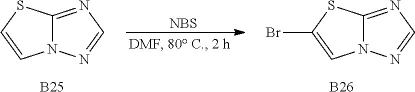

A mixture of [1,2,4]triazolo[3,2-b][1,3]thiazole (B25; 2 g, 16 mmol) and N-bromosuccinimide (5.57 g, 31 mmol) in dimethylformamide (20 mL) was stirred for 2 h at 80° C., then cooled to 20° C. The resulting mixture was poured onto water (100 mL) and extracted with ethyl acetate (3×100 mL). The combined organic layers were washed with brine (3×100 mL), dried over anhydrous sodium sulfate, filtered, and concentrated under reduced pressure. The residue was purified by silica gel column chromatography, eluting with petroleum ether/ethyl acetate (1:1) to afford 5-bromo-[1,2,4]triazolo[3,2-b][1,3]thiazole (B26; 1 g) as a solid. LCMS (ES, m/z): 204/206 [M+H]⁺. ¹H NMR (400 MHz, Methanol-d₄) δ 8.42 (s, 1H), 8.27 (s, 1H).

### Synthesis of Intermediate B28

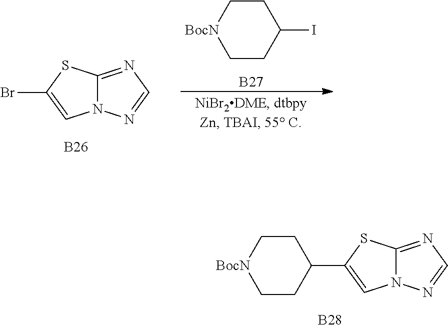 [SMILES: "B26" ; Brc1cn2ncnc2s1 ; "B27" ; "B28" ; CC(C)(C)OC(=O)N1CCC(I)CC1 ; CC(C)(C)OC(=O)N1CCC(c2cn3ncnc3s2)CC1]

A mixture of 4,4′-di-tert-butyl-2,2′-dipyridyl (387 mg, 1.44 mmol) in dimethylacetamide (20 mL) was treated with nickel(II) bromide ethylene glycol dimethyl ether complex (508 mg, 1.44 mmol) in portions at 25° C. under an argon atmosphere, and the resulting mixture was stirred for 20 min. Next, zinc (1.57 g, 24 mmol), tetrabutylammonium iodide (444 mg, 1.2 mmol), 5-bromo-[1,2,4]triazolo[3,2-b][1,3]thiazole (B26; 1 g, 4.8 mmol), and tert-butyl 4-iodopiperidine-1-carboxylate (B27; 2.24 g, 7.2 mmol) were added in portions at 25° C., and the resulting mixture was then heated 55° C. for 2 h. The mixture then cooled to 25° C. and filtered. The filtrate was poured onto water (100 mL) and extracted with ethyl acetate (3×100 mL). The combined organic layers were washed with brine (2×100 mL), dried over anhydrous sodium sulfate, filtered, and concentrated under reduced pressure. The residue was purified by silica gel column chromatography, eluting with petroleum ether/ethyl acetate (5:1) to afford tert-butyl 4-[[1,2,4]triazolo[3,2-b][1,3]thiazol-5-yl]piperidine-1-carboxylate (B28; 140 mg) as a solid. LCMS (ES, m/z): 309 [M+H].

### Synthesis of Intermediate B30

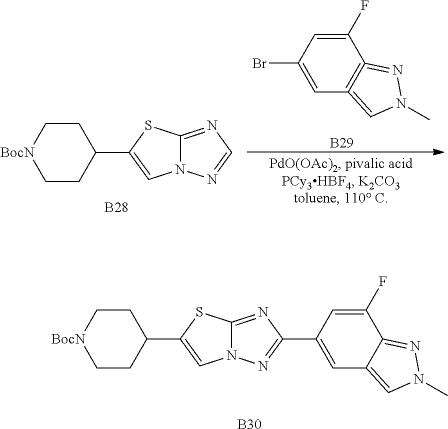

A solution of tert-butyl 4-[[1,2,4]triazolo[3,2-b][1,3]thiazol-5-yl]piperidine-1-carboxylate (B28; 140 mg, 0.45 mmol), 5-bromo-7-fluoro-2-methylindazole (B29; 102 mg, 0.45 mmol), Pd(OAc)₂ (10 mg, 0.05 mmol), pivalic acid (30 mg, 0.29 mmol), tricyclohexylphosphine tetrafluoroborate (33 mg, 0.09 mmol) and potassium carbonate (369 mg, 2.670 mmol) in toluene (3 mL) was stirred for 16 h at 110° C. under a nitrogen atmosphere. The mixture was then cooled to 25° C. and concentrated under reduced pressure. The residue was purified by silica gel column chromatography, eluting with hexanes/ethyl acetate (1:1) to afford tert-butyl-4-[2-(7-fluoro-2-methylindazol-5-yl)-[1,2,4]triazolo[3,2-b][1,3]thiazol-5-yl]piperidine-1-carboxylate (B30; 25 mg) as an oil. LCMS (ES, m/z): 457 [M+H]⁺.

### Synthesis of Compound 103

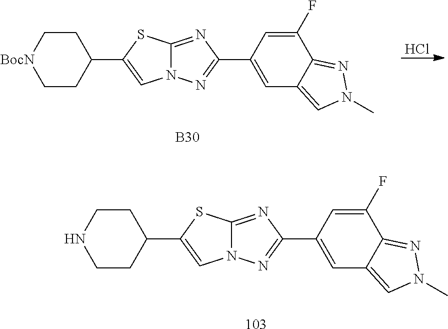

A solution of tert-butyl 4-[2-(7-fluoro-2-methylindazol-5-yl)-[1,2,4]triazolo[3,2-b][1,3]thiazol-5-yl]piperidine-1-carboxylate (B30; 25 mg, 0.05 mmol) and HCl in 1,4-dioxane (2 mL) was stirred for 30 min at 25° C. The resulting mixture was concentrated under reduced pressure and purified by preparative HPLC (Condition 1, Gradient 1), to afford 7-fluoro-2-methyl-5-[5-(piperidin-4-yl)-[1,2,4]triazolo[3,2-b][1,3]thiazol-2-yl]indazole (Compound 103; 5.9 mg) as a solid. LCMS (ES, m/z): 357 [M+H]⁺. ¹H NMR (400 MHz, DMSO-d₆) δ 8.65 (d, J=2.8 Hz, 1H), 8.28 (s, 1H), 7.89 (d, J=1.3 Hz, 1H), 7.31 (dd, J=12.4, 1.3 Hz, 1H), 4.27 (s, 3H), 3.31 (s, 1H), 3.13 (s, 2H), 3.00 (d, J=12.3 Hz, 2H), 1.91 (d, J=12.2 Hz, 2H), 1.64-1.56 (m, 1H), 1.59-1.51 (m, 1H).

### Example 6: Synthesis of Compound 110

### Synthesis of Intermediate B32

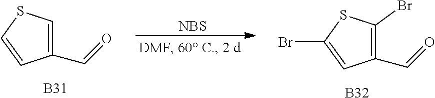 [SMILES: "B31" ; O=Cc1ccsc1 ; "NBS" ; "B32" ; O=Cc1cc(Br)sc1Br]

A solution of 3-thiophenecarboxaldehyde (B31; 15 g, 134 mmol) in dimethylformamide (150 mL) was treated with N-bromosuccinimide (47.6 g, 267 mmol) in portions at room temperature under a nitrogen atmosphere, and the resulting mixture was stirred for 2 days at 60° C. The reaction was quenched with sodium sulfite at room temperature and extracted with ethyl acetate (2×200 mL). The combined organic layers were washed with brine (2×200 mL), dried over anhydrous sodium sulfate, filtered, and concentrated under reduced pressure to give a residue. The residue was purified by reverse phase flash chromatography on a C18 silica gel column, eluting with methanol (60% to 80% gradient over 10 min) in water, to afford 2,5-dibromothiophene-3-carbaldehyde (B32; 3.5 g) as an oil. LCMS (ES, m/z): 270 [M+H]⁺.

### Synthesis of Intermediate B34

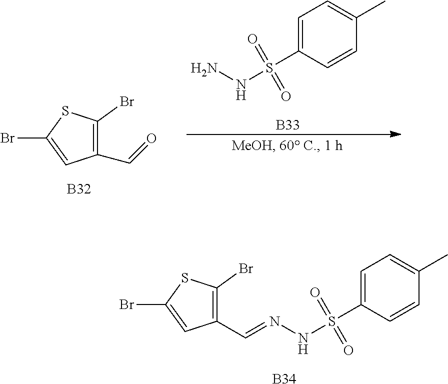

A solution of 2,5-dibromothiophene-3-carbaldehyde (B32; 3.5 g, 0.013 mmol) in methanol (35 mL) was treated with 4-toluenesulfonyl hydrazide (B33; 2.17 g, 0.012 mmol) dropwise at room temperature under a nitrogen atmosphere, and the resulting mixture was stirred for 1 h at 60° C. The reaction was quenched with methanol at room temperature, and the precipitated solids were collected by filtration and washed with methanol (2×5 mL), to afford N-[(1E)-(2,5-dibromothiophen-3-yl) methylidene]-4-methylbenzenesulfonohydrazide (B34; 4.5 g) as a solid. LCMS (ES, m/z): 438 [M+H]⁺.

### Synthesis of Intermediate B35

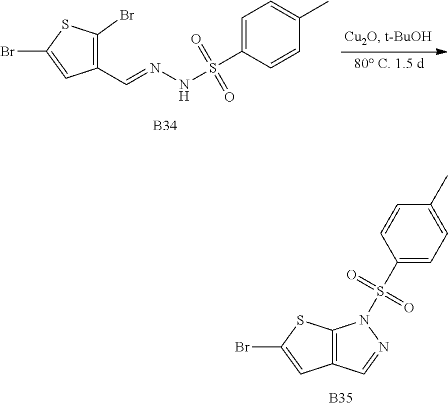

A solution of N-[(1E)-(2,5-dibromothiophen-3-yl) methylidene]-4-methylbenzenesulfonohydrazide (B34; 7 g, 16 mmol) in tert-butanol (70 mL) was treated with copper(I) oxide (2.3 g, 16 mmol) in portions at room temperature under a nitrogen atmosphere, and the resulting mixture was stirred overnight at 80° C. under a nitrogen atmosphere. The reaction was quenched with water at room temperature and extracted with ethyl acetate (2×100 mL). The combined organic layers were washed with brine (2×100 mL), dried over anhydrous sodium sulfate, filtered, and concentrated under reduced pressure. The residue was purified by silica gel column chromatography, eluting with petroleum ether/ethyl acetate (8:1) to afford 5-bromo-1-(4-methylbenzenesulfonyl) thieno[2,3-c]pyrazole (B35; 4.9 g) as a solid. LCMS (ES, m/z): 357 [M+H]⁺.

### Synthesis of Intermediate B36

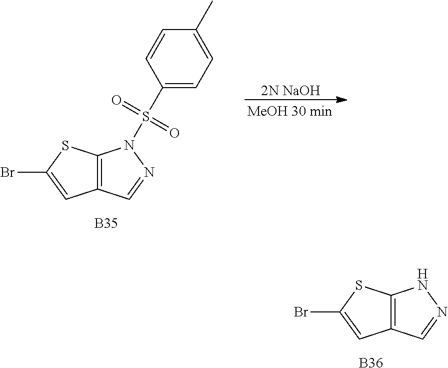

A solution of 5-bromo-1-(4-methylbenzenesulfonyl) thieno[2,3-c] pyrazole (B35; 950 mg, 2.66 mmol) in methanol (2 mL) was treated with sodium hydroxide solution (2 mL, 4 mmol) in portions at room temperature under a nitrogen atmosphere. The resulting mixture was stirred for 30 min at room temperature, and was then quenched with water. The resulting mixture was extracted with ethyl acetate (2×4 mL), and the combined organic layers were washed with brine (2×4 mL), dried over anhydrous sodium sulfate, filtered, and concentrated under reduced pressure, to afford 5-bromo-1H-thieno[2,3-c] pyrazole (B36; 620 mg) as a solid. LCMS (ES, m/z): 203 [M+H]⁺.

### Synthesis of Intermediate B37

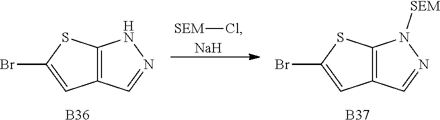

A solution of 5-bromo-1H-thieno[2,3-c] pyrazole (B36; 2.5 g, 12.3 mmol) in dimethylformamide (75 mL) was treated with sodium hydride (985 mg, 41 mmol) in portions at 0° C. under a nitrogen atmosphere. The reaction mixture was then irradiated with a microwave for 1 h at 0° C. Next, 2-(trimethylsilyl)ethoxymethyl chloride (3.08 g, 18.5 mmol) was added to the mixture at 0° C. under nitrogen atmosphere over 2 h. The reaction was quenched with water/ice at room temperature, and extracted with ethyl acetate (2×10 mL). The combined organic layers were washed with brine (2×10 mL), dried over anhydrous sodium sulfate, filtered, and concentrated under reduced pressure. The residue was purified by silica gel column chromatography, eluting with petroleum ether/ethyl acetate (9:1) to afford 5-bromo-1-[[2-(trimethylsilyl) ethoxy] methyl] thieno[2,3-c] pyrazole (B37; 4 g) as a solid. LCMS (ES, m/z): 333 [M+H]⁺.

### Synthesis of Intermediate B39

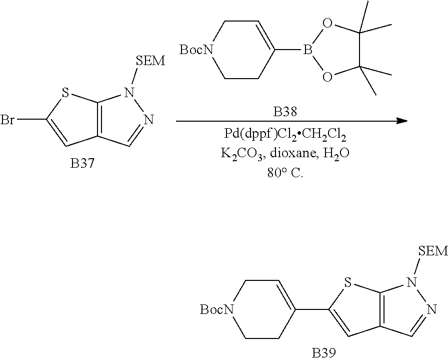 [SMILES: "B37" ; C[Si](C)(C)CCOCn1ncc2cc(Br)sc21 ; "B38" ; "B39" ; CC(C)(C)OC(=O)N1CC=C(B2OC(C)(C)C(C)(C)O2)CC1 ; CC(C)(C)OC(=O)N1CC=C(c2cc3cnn(COCC[Si](C)(C)C)c3s2)CC1]

A solution of 5-bromo-1-[[2-(trimethylsilyl) ethoxy] methyl] thieno[2,3-c] pyrazole (B37; 400 mg, 1.2 mmol) and tert-butyl 4-(4,4,5,5-tetramethyl-1,3,2-dioxaborolan-2-yl)-3,6-dihydro-2H-pyridine-1-carboxylate (B38; 557 mg, 1.8 mmol) in dioxane (4 mL) was treated with Pd(dppf)Cl₂·CH₂Cl₂ (49 mg, 0.06 mmol), potassium carbonate (498 mg, 3.6 mmol) and water (4 mL) in portions at room temperature under a nitrogen atmosphere. The resulting mixture was stirred overnight at 80° C., then filtered, and concentrated under reduced pressure. The residue was purified by silica gel column chromatography, eluting with petroleum ether/ethyl acetate (10:1) to afford tert-butyl 4-(1-[[2-(trimethylsilyl) ethoxy]methyl] thieno[2,3-c] pyrazol-5-yl)-3,6-dihydro-2H-pyridine-1-carboxylate (B39; 430 mg) as a solid. LCMS (ES, m/z): 436 [M+H]⁺.

### Synthesis of Intermediate B40

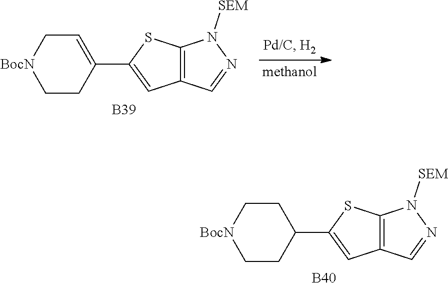 [SMILES: "B39" ; CC(C)(C)OC(=O)N1CC=C(c2cc3cnn(COCC[Si](C)(C)C)c3s2)CC1 ; "B40" ; CC(C)(C)OC(=O)N1CCC(c2cc3cnn(COCC[Si](C)(C)C)c3s2)CC1]

A solution of tert-butyl 4-(1-[[2-(trimethylsilyl) ethoxy] methyl] thieno[2,3-c] pyrazol-5-yl)-3,6-dihydro-2H-pyridine-1-carboxylate (B39; 400 mg, 0.92 mmol) in methanol (8 mL) was treated with palladium on carbon (400 mg, 3.78 mmol) in portions at room temperature under a nitrogen atmosphere. The resulting mixture was stirred for 3 days at 40° C. under a hydrogen atmosphere (40 atm). The precipitated solids were then collected by filtration and washed with methanol (2×2 mL), and the filtrate was concentrated under reduced pressure to afford tert-butyl 4-(1-[[2-(trimethylsilyl) ethoxy] methyl] thieno[2,3-c] pyrazol-5-yl) piperidine-1-carboxylate (B40; 340 mg) as a solid. LCMS (ES, m/z): 438 [M+H]⁺.

### Synthesis of Intermediate B41

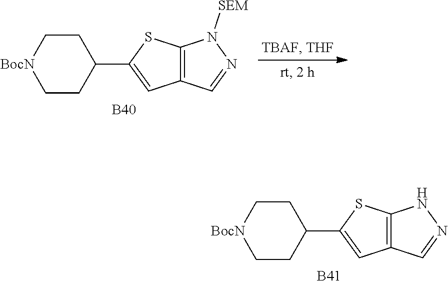 [SMILES: "B40" ; CC(C)(C)OC(=O)N1CCC(c2cc3cnn(COCC[Si](C)(C)C)c3s2)CC1 ; "B41" ; CC(C)(C)OC(=O)N1CCC(c2cc3cn[nH]c3s2)CC1]

A solution of tert-butyl 4-(1-[[2-(trimethylsilyl) ethoxy] methyl] thieno[2,3-c] pyrazol-5-yl) piperidine-1-carboxylate (B40; 350 mg, 0.8 mmol) in THE (4 mL) was treated with tetrabutylammonium fluoride (4 mL, 4 mmol) in portions at room temperature under a nitrogen atmosphere. The resulting mixture was stirred for 2 h and was quenched with water at room temperature. The resulting mixture was extracted with ethyl acetate (2×5 mL), and the combined organic layers were washed with water (7×5 mL), dried over anhydrous sodium sulfate, filtered, and concentrated under reduced pressure. The residue was purified by silica gel column chromatography, eluting with petroleum ether/ethyl acetate (5:1) to afford tert-butyl 4-[1H-thieno[2,3-c] pyrazol-5-yl] piperidine-1-carboxylate (B41; 260 mg) as a solid. LCMS (ES, m/z): 308 [M+H]⁺.

### Synthesis of Intermediate B43

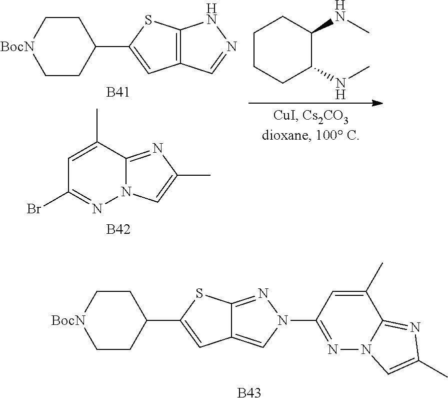

A solution of tert-butyl 4-[1H-thieno[2,3-c] pyrazol-5-yl] piperidine-1-carboxylate (B41; 125 mg, 0.41 mmol) and 6-bromo-2,8-dimethylimidazo[1,2-b] pyridazine (B42; 138 mg, 0.61 mmol) in dioxane (2 mL) was treated with copper(I) iodide (7.7 mg, 0.04 mmol), trans-N, N-dimethylcyclohexane-1,2-diamine (11.6 mg, 0.08 mmol) and cesium carbonate (397 mg, 1.2 mmol) in portions at room temperature under a nitrogen atmosphere. The resulting mixture was stirred overnight at 100° C., then filtered and concentrated under reduced pressure. The residue was purified by silica gel column chromatography, eluting with petroleum ether/ethyl acetate (3:1) to afford tert-butyl 4-(2-[2,8-dimethylimidazo[1,2-b] pyridazin-6-yl] thieno[2,3-c] pyrazol-5-yl) piperidine-1-carboxylate (B43; 63 mg) as a solid. LCMS (ES, m/z): 453 [M+H]⁺.

### Synthesis of Compound 110

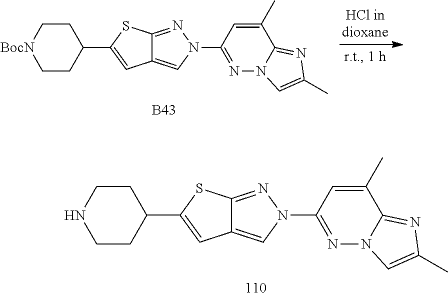 [SMILES: "B43" ; Cc1cn2nc(-n3cc4cc(C5CCN(C(=O)OC(C)(C)C)CC5)sc4n3)cc(C)c2n1 ; Cc1cn2nc(-n3cc4cc(C5CCNCC5)sc4n3)cc(C)c2n1 ; "110"]

A solution of tert-butyl 4-(2-[2,8-dimethylimidazo[1,2-b] pyridazin-6-yl] thieno[2,3-c]pyrazol-5-yl) piperidine-1-carboxylate (B43; 28 mg, 0.06 mmol) and HCl in 1,4-dioxane (2 mL) was stirred at room temperature under a nitrogen atmosphere for 1 h. The mixture was then filtered and concentrated under reduced pressure. The crude product was purified by preparative HPLC (Condition 2, Gradient 1) to afford 4-(2-[2,8-dimethylimidazo[1,2-b]pyridazin-6-yl] thieno[2,3-c] pyrazol-5-yl) piperidine (Compound 110; 7.4 mg) as a solid. LCMS (ES, m/z): 353 [M+H]+. ¹H NMR (400 MHz, DMSO-d6) δ 8.11 (s, 1H), 8.00 (s, 1H), 7.73 (s, 1H), 6.94 (s, 1H), 3.03 (s, 2H), 2.94 (s, 1H), 2.66 (s, 1H), 2.62 (s, 3H), 2.58 (s, 1H), 2.39 (s, 3H), 1.94 (d, J=12.4 Hz, 2H), 1.56 (qd, J=12.1, 3.9 Hz, 2H).

### Example 7: Synthesis of Compound 104

### Synthesis of Intermediate B44

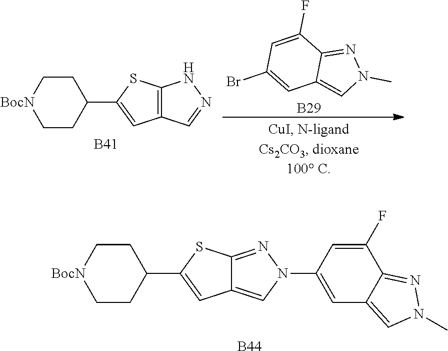

A solution of tert-butyl 4-[1H-thieno[2,3-c] pyrazol-5-yl] piperidine-1-carboxylate (B41 from Example 7; 260 mg, 0.85 mmol) and 5-bromo-7-fluoro-2-methylindazole (B29; 291 mg, 1.27 mmol) in dioxane (3 mL) was treated with copper(I) iodide (16 mg, 0.09 mmol), trans-N,N-dimethylcyclohexane-1,2-diamine (24 mg, 0.17 mmol) and cesium carbonate (827 mg, 2.54 mmol) in portions at room temperature under a nitrogen atmosphere. The resulting mixture was stirred overnight at 100° C., then filtered and concentrated under reduced pressure. The residue was purified by silica gel column chromatography, eluting with petroleum ether/ethyl acetate (3:1) to afford tert-butyl 4-[2-(7-fluoro-2-methylindazol-5-yl) thieno[2,3-c] pyrazol-5-yl] piperidine-1-carboxylate (B44; 128 mg) as a solid. LCMS (ES, m/z): 456 [M+H]⁺.

### Synthesis of Compound 104

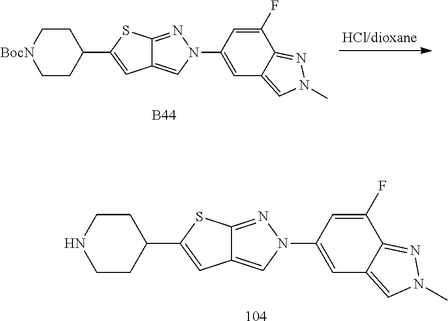 [SMILES: "B44" ; Cn1cc2cc(-n3cc4cc(C5CCN(C(=O)OC(C)(C)C)CC5)sc4n3)cc(F)c2n1 ; Cn1cc2cc(-n3cc4cc(C5CCNCC5)sc4n3)cc(F)c2n1 ; "104"]

A solution of tert-butyl 4-[2-(7-fluoro-2-methylindazol-5-yl) thieno[2,3-c] pyrazol-5-yl]piperidine-1-carboxylate (B44; 43 mg, 0.09 mmol) and HCl in 1,4-dioxane (2 mL) was stirred for 1 h at room temperature under a nitrogen atmosphere, then filtered and concentrated under reduced pressure. The residue was purified by preparative HPLC (Condition 1, Gradient 2), to afford 7-fluoro-2-methyl-5-[5-(piperidin-4-yl) thieno[2,3-c]pyrazol-2-yl] indazole (B45; 6.3 mg) as a solid. LCMS (ES, m/z): 356 [M+H]⁺. ¹H NMR (400 MHz, DMSO-d₆) δ 8.59 (d, J=2.8 Hz, 1H), 8.01 (s, 1H), 7.73 (d, J=1.8 Hz, 1H), 7.66 (dd, J=12.6, 1.8 Hz, 1H), 6.97 (d, J=1.1 Hz, 1H), 4.24 (s, 3H), 3.04 (d, J=12.2 Hz, 2H), 2.95 (t, J=11.7 Hz, 1H), 2.61 (dd, J=12.8, 10.4 Hz, 2H), 1.96 (d, J=12.6 Hz, 2H), 1.59 (dd, J=12.3, 3.8 Hz, 1H), 1.53 (dd, J=12.0, 3.8 Hz, 1H), 1.24 (s, 1H).

### Example 8: Synthesis of Compound 106

### Synthesis of Intermediate B46

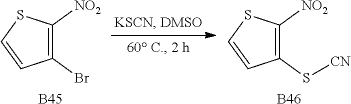 [SMILES: "B45" ; O=[N+]([O-])c1sccc1Br ; "B46" ; N#CSc1ccsc1[N+](=O)[O-]]

A mixture of 3-bromo-2-nitrothiophene (B45; 19 g, 91.1 mmol) and potassium thiocyanate (7.4 g, 273 mmol) in dimethylsulfoxide (180 mL) was stirred for 2 h at 60° C. under an atmosphere of nitrogen, then filtered and concentrated, to afford 2-nitro-3-thiocyanatothiophene (B46; 15.1 g) as a solid. LCMS (ES, m/z): 187 [M+H+41]⁺.

### Synthesis of Intermediate B47

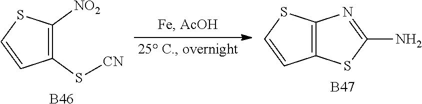 [SMILES: "B46" ; N#CSc1ccsc1[N+](=O)[O-] ; "B47" ; Nc1nc2sccc2s1]

A mixture of 2-nitro-3-thiocyanatothiophene (B46; 15 g, 80.6 mmol) and iron (26 g, 403 mmol) in acetic acid (350 mL) was stirred overnight at 25° C. under an atmosphere of nitrogen. Water (1 L) was then added, and the pH value of the solution was adjusted to 8 with sodium carbonate. The resulting solution was extracted with ethyl acetate (3×100 mL) and the combined organic layers were concentrated to afford thieno[2,3-d]thiazol-2-amine (B47; 10 g) as a solid. LCMS (ES, m/z): 157 [M+H]⁺.

### Synthesis of Intermediate B48

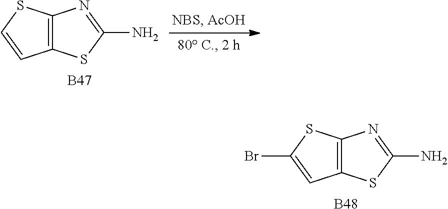

A mixture of thieno[2,3-d][1,3]thiazol-2-amine (B47; 6.5 g, 41.6 mmol), N-bromosuccinimide (7.4 g, 41.6 mmol), and acetic acid (200 mL) was stirred for 2 h at 80° C. under an atmosphere of nitrogen, and then cooled to 25° C. The pH value of the solution was adjusted to 8 with sodium carbonate, and extracted with ethyl acetate (3×100 mL). The combined organic layers were then concentrated and the residue was purified by silica gel column chromatography eluting with ethyl acetate/petroleum ether (1:1) to afford 5-bromothieno[2,3-d][1,3]thiazol-2-amine (B48; 3.3 g) as a solid. LCMS (ES, m/z): 235 [M+H]⁺.

### Synthesis of Intermediate B49

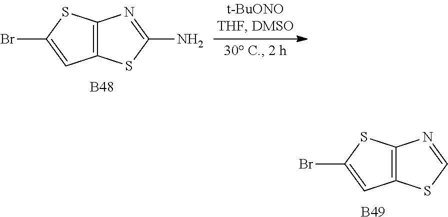

A mixture of 5-bromothieno[2,3-d][1,3]thiazol-2-amine (B48; 6 g, 25.5 mmol), THF (60 mL), and DMSO (0.2 g, 2.55 mmol) was treated with tert-butyl nitrite (3.95 g) dropwise with stirring at 0° C. The resulting solution was stirred for 2 h at 30° C., and was then concentrated. The residue was purified by silica gel column chromatography eluting with ethyl acetate/petroleum ether (3:1), to afford 5-bromothieno[2,3-d][1,3]thiazole (B49; 2.6 g) as a solid. LCMS (ES, m/z): 220 [M+H]⁺.

### Synthesis of Intermediate B51

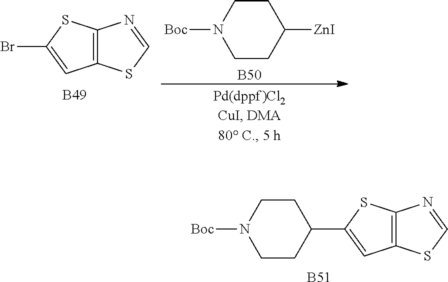

A mixture of 5-bromothieno[2,3-d][1,3]thiazole (B49; 1.2 g, 5.45 mmol), Pd(dppf)Cl₂ (0.4 g), copper(I) iodide (0.21 g, 1.09 mmol), and [1-(tert-butoxycarbonyl)piperidin-4-yl](iodo)zinc (B50; 15 mL) in dimethylacetamide (20 mL) was stirred for 5 h at 80° C. under an atmosphere of nitrogen. The reaction mixture was then cooled to 25° C. and quenched with water/ice. The resulting solution was extracted with ethyl acetate (3×20 mL) and the combined organic layers were concentrated, and purified by Flash-Prep-HPLC (IntelFlash-1) on a C₁₈ silica gel column, eluting with acetonitrile in water (ACN/H₂O=3:7 increasing to 7:3 within 30 min), to afford tert-butyl 4-[thieno[2,3-d][1,3]thiazol-5-yl]piperidine-1-carboxylate (B51; 260 mg) as a solid. LCMS (ES, m/z): 325 [M+H]⁺.

### Synthesis of Intermediate B55

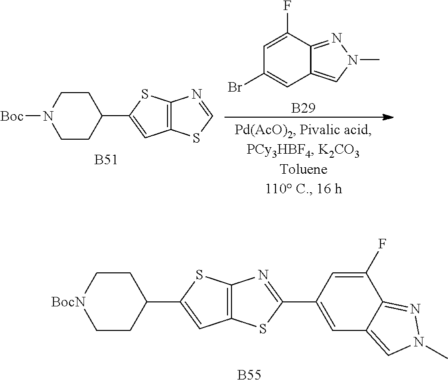 [SMILES: "B51" ; CC(C)(C)OC(=O)N1CCC(c2cc3scnc3s2)CC1 ; "B55" ; Cn1cc2cc(-c3nc4sc(C5CCN(C(=O)OC(C)(C)C)CC5)cc4s3)cc(F)c2n1 ; "B29" ; Cn1cc2cc(Br)cc(F)c2n1]

A mixture of tert-butyl 4-[thieno[2,3-d][1,3]thiazol-5-yl]piperidine-1-carboxylate (B51 from Example 9; 90 mg, 0.27 mmol), 5-bromo-7-fluoro-2-methylindazole (B29; 95.3 mg, 0.41 mmol), Pd(AcO)₂ (6.2 mg, 0.03 mmol), pivalic acid (18.4 mg, 0.18 mmol), tricyclohexylphosphine tetrafluoroborate (20.4 mg, 0.05 mmol), and potassium carbonate (230 mg, 1.66 mmol), in toluene (3 mL) was stirred for 16 h at 110° C. under an atmosphere of nitrogen. The resulting mixture was concentrated and purified by silica gel column chromatography eluting with ethyl acetate/petroleum ether (1:1) to afford tert-butyl 4-[2-(7-fluoro-2-methylindazol-5-yl)thieno[2,3-d] [1,3]thiazol-5-yl]piperidine-1-carboxylate (B55; 50 mg) as a solid. LCMS (ES, m/z): 473 [M+H]⁺.

### Synthesis of Compound 106

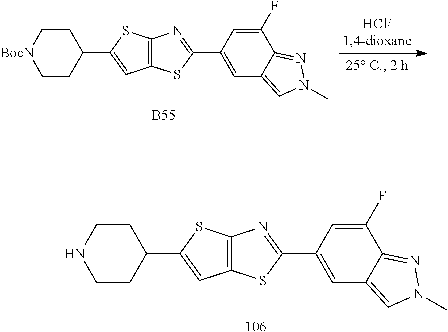 [SMILES: "B55" ; Cn1cc2cc(-c3nc4sc(C5CCN(C(=O)OC(C)(C)C)CC5)cc4s3)cc(F)c2n1 ; Cn1cc2cc(-c3nc4sc(C5CCNCC5)cc4s3)cc(F)c2n1 ; "106"]

A mixture of tert-butyl 4-[2-(7-fluoro-2-methylindazol-5-yl)thieno[2,3-d][1,3]thiazol-5-yl]piperidine-1-carboxylate (B55; 50 mg, 0.11 mmol), and HCl in 1,4-dioxane (1 mL) was stirred for 2 h at 25° C., then concentrated and purified by preparative HPLC (Condition 2, Gradient 3) to afford 7-fluoro-2-methyl-5-[5-(piperidin-4-yl)thieno[2,3-d][1,3]thiazol-2-yl]indazole (Compound 106; 11.8 mg) as a solid. LCMS (ES, m/z): 373 [M+H]⁺. ¹H NMR (400 MHz, DMSO-d₆) δ 8.61 (d, J=2.7 Hz, 1H), 8.21 (d, J=1.3 Hz, 1H), 7.64 (dd, J=12.5, 1.4 Hz, 1H), 7.28 (d, J=1.0 Hz, 1H), 4.24 (s, 3H), 3.00 (ddt, J=20.3, 11.8, 3.5 Hz, 3H), 2.61 (td, J=12.1, 2.4 Hz, 2H), 1.89-1.90 (m, 2H), 1.55 (qd, J=12.2, 3.9 Hz, 2H).

### Example 9: Synthesis of Compound 107

### Synthesis of Intermediate B57

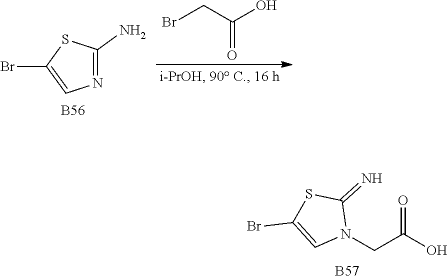 [SMILES: "B56" ; Nc1ncc(Br)s1 ; O=C(O)CBr ; "B57" ; N=c1sc(Br)cn1CC(=O)O]

A solution of 5-bromo-1,3-thiazol-2-amine (B56; 23 g, 128 mmol) and 2-bromoacetic acid (17.9 g, 128 mmol) in isopropanol (200 mL) was stirred for 16 h at 90° C. The mixture was then cooled to 25° C., filtered, and concentrated under reduced pressure to afford (2-amino-5-bromo-2H-1,3-thiazol-3-yl)acetic acid (B57; 27 g) as an oil. LCMS: (ES, m/z): 237[M+H]⁺.

### Synthesis of Intermediate B58

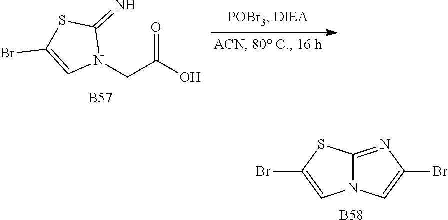

A solution of (2-amino-5-bromo-2H-1,3-thiazol-3-yl)acetic acid (B57; 27 g, 113 mmol) and diisopropylethylamine (14.6 g, 113 mmol) in acetonitrile (300 mL) was stirred for 30 min at 25° C. Phosphoryl bromide (129.5 g, 450 mmol) was then added and the mixture was stirred for 16 h at 80° C., and then cooled to 25° C. The reaction was quenched with aqueous sodium carbonate (200 mL) at 30° C. The aqueous layer was extracted with ethyl acetate (3×200 mL) and concentrated under reduced pressure. The residue was purified by reverse phase flash chromatography on a silica gel column eluting with methanol (10% to 50% gradient over 10 min) in water, to afford 2,6-dibromoimidazo[2,1-b][1,3] thiazole (B58; 2 g) as a solid. LCMS: (ES, m/z): 281[M+H]⁺.

### Synthesis of Intermediate B60

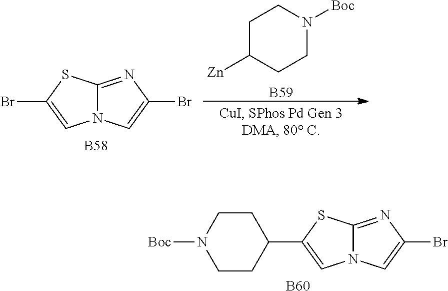

A solution of 2,6-dibromoimidazo [2,1-b] [1,3] thiazole (B58; 500 mg, 1.8 mmol), copper(I) iodide (34 mg, 0.18 mmol) and SPhos-Pd Gen-3 (138 mg, 0.18 mmol) in dimethylacetamide was treated with [1-(tert-butoxycarbonyl) piperidin-4-yl] zinc (B59; 443 mg, 1.8 mmol) under a nitrogen atmosphere, and the mixture was stirred for 16 h at 80° C. The mixture was then cooled to 25° C., and the precipitated solids were collected by filtration and washed with ethyl acetate and H₂O (3×40 mL). The mixture was then concentrated under vacuum, and purified by reverse phase flash chromatography on a silica gel column eluting with methanol (10% to 50% gradient over 10 min) in water, to afford tert-butyl 4-[6-bromoimidazo[2,1-b][1,3] thiazol-2-yl] piperidine-1-carboxylate (B60; 30 mg) as a solid. LCMS: (ES, m/z): 385[M+H]⁺.

### Synthesis of Intermediate B61

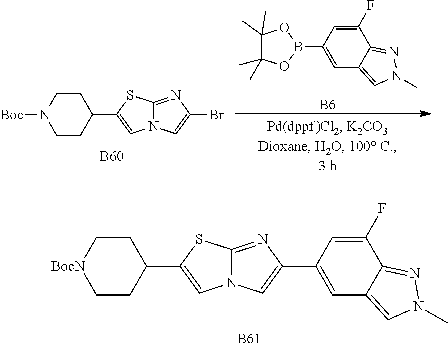

A solution of tert-butyl 4-[6-bromoimidazo[2,1-b][1,3]thiazol-2-yl]piperidine-1-carboxylate (B60; 40 mg, 0.1 mmol), 7-fluoro-2-methyl-5-(4,4,5,5-tetramethyl-1,3,2-dioxaborolan-2-yl)indazole (B6; 31 mg, 0.1 mmol), Pd(dppf)Cl₂ (15.2 mg, 0.02 mmol) and potassium carbonate (43 mg, 0.3 mmol) in dioxane (1.5 mL) and H₂O (0.3 mL) was stirred for 3 h at 100° C. The mixture was then cooled to 25° C. and extracted with ethyl acetate (3×10 mL). The resulting mixture was concentrated under vacuum and purified by reverse phase flash chromatography on a silica gel column eluting with methanol (10% to 50% gradient over 10 min) in water, to afford tert-butyl 4-[6-(7-fluoro-2-methylindazol-5-yl)imidazo [2,1-b][1,3]thiazol-2-yl]piperidine-1-carboxylate (B61; 18 mg) as a solid. LCMS: (ES, m/z): 455[M+1]⁺.

### Synthesis of Compound 107

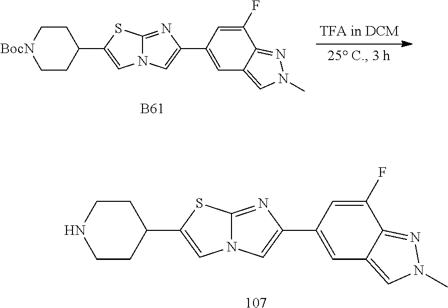 [SMILES: "B61" ; Cn1cc2cc(-c3cn4cc(C5CCN(C(=O)OC(C)(C)C)CC5)sc4n3)cc(F)c2n1 ; Cn1cc2cc(-c3cn4cc(C5CCNCC5)sc4n3)cc(F)c2n1 ; "107"]

A solution of tert-butyl-4-[6-(7-fluoro-2-methylindazol-5-yl)imidazo[2,1-b][1,3]thiazol-2-yl]piperidine-1-carboxylate (B60; 18 mg, 0.04 mmol) and trifluoroacetic acid (1 mL, 13.5 mmol) in dichloromethane (3 mL) was stirred for 3 h at 25° C. The resulting mixture was then concentrated under vacuum and purified by reverse phase flash chromatography on a C₁s silica gel column, eluting with methanol (10% to 50% gradient over 10 min) in water, to afford 7-fluoro-2-methyl-5-[2-(piperidin-4-yl) imidazo [2,1-b] [1,3] thiazol-6-yl] indazole (Compound 107; 1.2 mg) as a solid. LCMS: (ES, m/z): 355[M+H]+. ¹H NMR: (400 MHz, DMSO-d₆, ppm): δ 8.48 (d, J=3.4 Hz, 2H), 8.02 (d, J=1.1 Hz, 1H), 7.57 (dd, J=13.2, 1.2 Hz, 1H), 6.88 (d, J=1.0 Hz, 1H), 4.20 (s, 3H), 3.09 (d, J=12.4 Hz, 2H), 2.91 (d, J=11.8 Hz, 1H), 2.02 (d, J=12.2 Hz, 2H), 1.61-1.48 (m, 2H), 1.24 (s, 1H).

### Example 10: Synthesis of Compound 112

### Synthesis of Intermediate B62

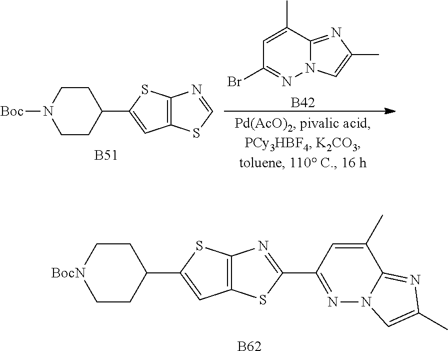 [SMILES: "B51" ; CC(C)(C)OC(=O)N1CCC(c2cc3scnc3s2)CC1 ; "B62" ; Cc1cn2nc(-c3nc4sc(C5CCN(C(=O)OC(C)(C)C)CC5)cc4s3)cc(C)c2n1 ; "B42" ; Cc1cn2nc(Br)cc(C)c2n1]

A mixture of tert-butyl 4-[thieno[2,3-d][1,3]thiazol-5-yl]piperidine-1-carboxylate (B51 from Example 9; 90 mg, 0.28 mmol), 6-bromo-2,8-dimethylimidazo[1,2-b]pyridazine (B42; 94 mg, 0.42 mmol), Pd(AcO)₂ (6.2 mg, 0.028), pivalic acid (18.4 mg, 0.18 mmol), tricyclohexylphosphine tetrafluoroborate (20.4 mg, 0.05 mmol), and potassium carbonate (230 mg, 1.6 mmol) in toluene (3 mL) was stirred for 16 h at 110° C. under an atmosphere of nitrogen. The resulting mixture was concentrated and purified by silica gel column chromatography eluting with ethyl acetate/petroleum ether (1:1) to afford tert-butyl 4-(2-[2,8-dimethylimidazo[1,2-b]pyridazin-6-yl]thieno[2,3-d][1,3]thiazol-5-yl)piperidine-1-carboxylate (B62; 50 mg) as a solid. LCMS (ES, m/z): 470 [M+H]⁺.

### Synthesis of Compound 112

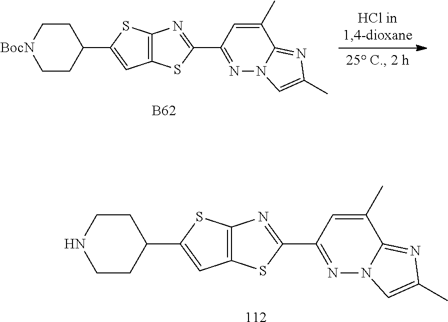 [SMILES: "B62" ; Cc1cn2nc(-c3nc4sc(C5CCN(C(=O)OC(C)(C)C)CC5)cc4s3)cc(C)c2n1 ; Cc1cn2nc(-c3nc4sc(C5CCNCC5)cc4s3)cc(C)c2n1 ; "112"]

A mixture of tert-butyl 4-(2-[2,8-dimethylimidazo[1,2-b]pyridazin-6-yl]thieno[2,3-d][1,3]thiazol-5-yl)piperidine-1-carboxylate (B62; 50 mg, 0.11 mmol) and HCl in 1,4-dioxane (1 mL) was stirred for 2 h at 25° C., and then concentrated and purified by preparative HPLC (Condition 2, Gradient 3), to afford 4-(2-[2,8-dimethylimidazo[1,2-b]pyridazin-6-yl]thieno[2,3-d][1,3]thiazol-5-yl)piperidine (Compound 112; 11.7 mg) as a solid. LCMS (ES, m/z): 370 [M+H]⁺. ¹H NMR (400 MHz, DMSO-d₆) δ 8.14 (d, J=1.0 Hz, 1H), 7.82 (d, J=1.3 Hz, 1H), 7.34 (d, J=1.0 Hz, 1H), 3.03 (dt, J=11.8, 6.2 Hz, 3H), 2.62 (dd, J=5.6, 1.8 Hz, 5H), 2.42 (s, 3H), 1.95 (d, J=11.8 Hz, 2H), 1.56 (qd, J=12.2, 3.9 Hz, 2H).

### Example 11: Synthesis of Compound 122

### Synthesis of Intermediate B63

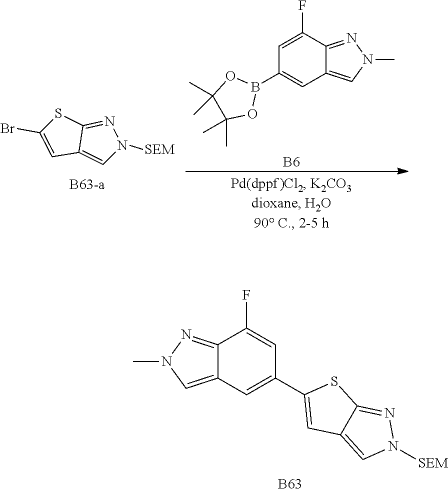

A mixture of 5-bromo-2-[[2-(trimethylsilyl)ethoxy]methyl]thieno[3,2-c]pyrazole (B63-a; 500 mg, 1.5 mmol), 7-fluoro-2-methyl-5-(4,4,5,5-tetramethyl-1,3,2-dioxaborolan-2-yl)indazole (B6; 414 mg, 1.5 mmol), potassium carbonate (622 mg, 4.5 mmol), and Pd(dppf)Cl₂ (183 mg, 0.23 mmol) in dioxane (15 mL) was purged with nitrogen for 1 min, then heated to 90° C. for 2-5 h. The resulting mixture was concentrated under reduced pressure and extracted with ethyl acetate (3×50 mL). The combined organic layers were washed with brine, dried over anhydrous sodium sulfate, filtered, and concentrated under reduced pressure. The residue was purified by silica gel column chromatography, eluting with acetate/petroleum ether (2:1) to afford (7-fluoro-2-methyl-5-(2-[[2-(trimethylsilyl)ethoxy]methyl]thieno[2,3-c]pyrazol-5-yl)indazole (B63; 310 mg) as a solid. LCMS (ES, m/z): 403 [M+H]⁺.

### Synthesis of Intermediate B64

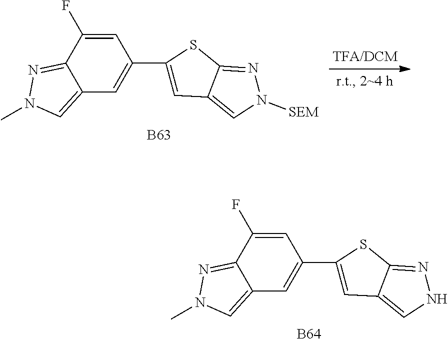 [SMILES: "B63" ; Cn1cc2cc(-c3cc4cn(COCC[Si](C)(C)C)nc4s3)cc(F)c2n1 ; "B64" ; Cn1cc2cc(-c3cc4c[nH]nc4s3)cc(F)c2n1]

A mixture of 7-fluoro-2-methyl-5-(2-[[2-(trimethylsilyl)ethoxy]methyl]thieno[2,3-c]pyrazol-5-yl)indazole (B63; 300 mg, 0.75 mmol), and trifluoroacetic acid (3 mL) in dichloromethane (3 mL) was stirred at room temperature for 2-4 h, and then neutralized with non-saturated aqueous sodium carbonate. The resulting mixture was extracted with ethyl acetate and the combined organic layers were washed with non-saturated sodium carbonate solution, dried over anhydrous sodium sulfate, filtered, and concentrated under reduced pressure, to afford 7-fluoro-2-methyl-5-[2H-thieno[2,3-c]pyrazol-5-yl]indazole (B64; 180 mg) as a solid. LCMS (ES, m/z): 273 [M+H]+.

### Synthesis of Intermediate B66

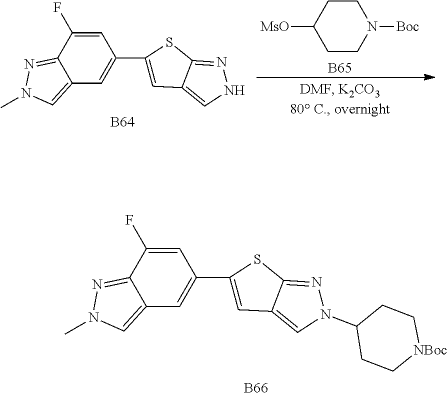 [SMILES: "B64" ; Cn1cc2cc(-c3cc4c[nH]nc4s3)cc(F)c2n1 ; Cn1cc2cc(-c3cc4cn(C5CCN(C(=O)OC(C)(C)C)CC5)nc4s3)cc(F)c2n1 ; "B66" ; "B65" ; CC(C)(C)OC(=O)N1CCC(OS(C)(=O)=O)CC1]

A mixture of 7-fluoro-2-methyl-5-[2H-thieno[2,3-c]pyrazol-5-yl]indazole (B64; 570 mg, 2 mmol) and tert-butyl 4-(methanesulfonyloxy)piperidine-1-carboxylate (B65; 877 mg, 3.1 mmol) in dimethylformamide (1 mL) was treated with potassium carbonate (38 mg, 0.28 mmol) at room temperature, and the mixture was then heated to 80° C. overnight under a nitrogen atmosphere. The resulting mixture was extracted with ethyl acetate and the combined organic layers were washed with brine, dried over anhydrous sodium sulfate, filtered, and concentrated under reduced pressure. The residue was purified by silica gel column chromatography, eluting with petroleum ether/ethyl acetate (1:1) to afford as a solid, which was further purified by chiral HPLC, to afford tert-butyl 4-[5-(7-fluoro-2-methylindazol-5-yl)thieno[2,3-c]pyrazol-2-yl]piperidine-1-carboxylate (B66; 764.4 mg) as a solid. LCMS (ES, m/z): 456 [M+H]⁺.

### Synthesis of Compound 122

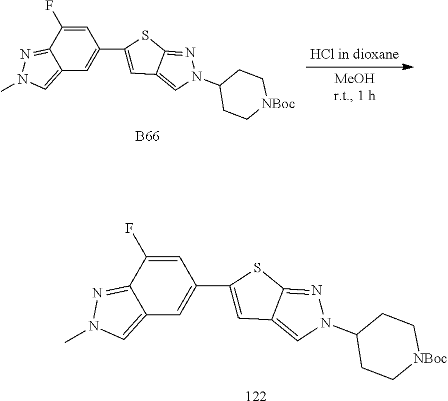 [SMILES: "B66" ; Cn1cc2cc(-c3cc4cn(C5CCN(C(=O)OC(C)(C)C)CC5)nc4s3)cc(F)c2n1 ; Cn1cc2cc(-c3cc4cn(C5CCN(C(=O)OC(C)(C)C)CC5)nc4s3)cc(F)c2n1 ; "122"]

tert-Butyl 4-[5-(7-fluoro-2-methylindazol-5-yl)thieno[2,3-c]pyrazol-1-yl]piperidine-1-carboxylate (B66; 50 mg, 0.11 mmol) was dissolved in methanol (1 mL), then HCl in 1,4-dioxane (4M, 2 mL) was added and the reaction mixture was stirred for 1 h at room temperature. The resulting mixture was then concentrated under reduced pressure, to afford 7-fluoro-2-methyl-5-[2-(piperidin-4-yl)thieno[2,3-c]pyrazol-5-yl]indazole (Compound 122; 42.7 mg) as a solid. LCMS (ES, m/z): 356 [M+H]⁺. ¹H NMR (400 MHz, DMSO-d₆, ppm) δ 9.07 (s, 1H), 8.89 (s, 1H), 8.51 (d, J=2.8 Hz, 1H), 7.85 (s, 1H), 7.70 (d, J=1.4 Hz, 1H), 7.54 (s, 1H), 7.48 (dd, J=13.0, 1.5 Hz, 1H), 4.71 (p, J=7.9, 7.4 Hz, 1H), 4.21 (s, 3H), 3.44 (d, J=12.8 Hz, 2H), 3.14 (q, J=7.0, 6.4 Hz, 2H), 2.28 (dt, J=9.6, 4.7 Hz, 4H).

### Example 12: Synthesis of Compound 123

### Synthesis of Intermediate B68

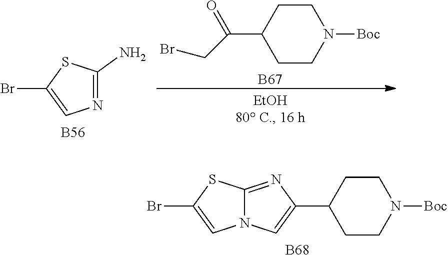

A mixture of 5-bromo-1,3-thiazol-2-amine (B56; 200 mg, 1.12 mmol) and tert-butyl 4-(2-bromoacetyl) cyclohexane-1-carboxylate (B67; 375 mg, 1.23 mmol) in ethanol (10 mL) was stirred for 16 h at 80° C. The mixture was then cooled to 25° C. and concentrated under reduced pressure. The residue was purified by reverse phase flash chromatography on a silica gel column eluting with methanol (10% to 50% gradient over 10 min) in water, to afford tert-butyl 4-[2-bromoimidazo[2,1-b] [1,3] thiazol-6-yl] piperidine-1-carboxylate (B68; 150 mg) as a solid. LCMS: (ES, m/z): 386[M+1]⁺.

### Synthesis of Intermediate B69

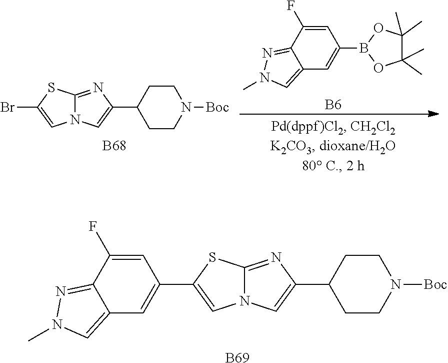

A solution of tert-butyl 4-[2-bromoimidazo[2,1-b] [1,3] thiazol-6-yl] piperidine-1-carboxylate (B68; 130 mg, 0.34 mmol), 7-fluoro-2-methyl-5-(4,4,5,5-tetramethyl-1,3,2-dioxaborolan-2-yl) indazole (B6; 111.5 mg, 0.40 mmol), Pd(dppf)Cl₂ (49 mg, 0.07 mmol) and potassium carbonate (140 mg, 1.0 mmol) in dioxane (5 mL) and H₂O (1 mL) was stirred for 2 h at 80° C. under an atmosphere of nitrogen. The mixture was then cooled to 25° C., and the aqueous layer was extracted with ethyl acetate and H₂O (3×15 mL). The resulting mixture was concentrated under vacuum and purified by reverse phase flash chromatography on a silica gel column eluting with methanol (10% to 50% gradient over 10 min) in water, to afford tert-butyl 4-[2-(7-fluoro-2-methylindazol-5-yl) imidazole[2,1-b] [1,3] thiazol-6-yl]piperidine-1-carboxylate (B69; 80 mg) as a solid. LCMS: (ES, m/z): 456[M+1]⁺.

### Synthesis of Compound 123

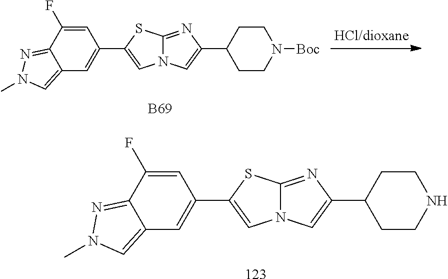 [SMILES: "B69" ; Cn1cc2cc(-c3cn4cc(C5CCN(C(=O)OC(C)(C)C)CC5)nc4s3)cc(F)c2n1 ; Cn1cc2cc(-c3cn4cc(C5CCNCC5)nc4s3)cc(F)c2n1 ; "123"]

A mixture of tert-butyl4-[2-(7-fluoro-2-methylindazol-5-yl) imidazo [2,1-b] [1,3]thiazol-6-yl] piperidine-1-carboxylate (B69; 80 mg, 0.18 mmol) and HCl (2 mL, 35 mmol) in dioxane (4 mL) was stirred for 3 h at 25° C., and then concentrated under vacuum. The residue was purified by reverse phase flash chromatography on a C₁₈ column, eluting with methanol (10% to 50% gradient over 10 min) in water, to afford 7-fluoro-2-methyl-5-[6-(piperidin-4-yl)imidazo[2,1-b][1,3]thiazol-2-yl]indazole (Compound 123; 14.2 mg) as a solid. LCMS: (ES, m/z): 356[M+1]+. ¹H NMR (400 MHz, DMSO-d₆, ppm) δ 8.51 (d, J=2.8 Hz, 1H), 8.37 (s, 1H), 7.69 (d, J=1.4 Hz, 1H), 7.52-7.44 (m, 2H), 4.22 (s, 3H), 3.01 (d, J=12.1 Hz, 2H), 2.67-2.54 (m, 3H), 1.88 (d, J=12.6 Hz, 2H), 1.50 (qd, J=12.3, 4.0 Hz, 2H). 1⁹F NMR (376 MHz, DMSO-d₆, ppm) 6-128.30.

### Example 13: Synthesis of Compound 124

### Synthesis of Intermediate B71

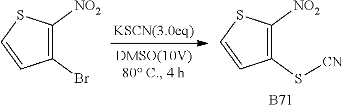 [SMILES: O=[N+]([O-])c1sccc1Br ; "B71" ; N#CSc1ccsc1[N+](=O)[O-]]

3-bromo-2-nitrothiophene (20 g, 96.14 mmol), DMSO (200 mL), and potassium thiocyanate (28.0 g, 288.43 mmol) were combined under an inert atmosphere of nitrogen. The reaction mixture was stirred for 4 h at 80° C., then quenched with a mixture of water and ice (200 mL), and extracted with ethyl acetate (3×200 mL). The organic layers were combined, washed with ½ saturated aqueous NaCl (3×200 mL) and saturated aqueous NaCl (1×200 mL), dried over anhydrous sodium sulfate, filtered, and concentrated in vacuo to afford [(2-nitrothiophen-3-yl)sulfanyl]formonitrile (17.7 g, 98.8%) as a solid.

### Synthesis of Intermediate B72

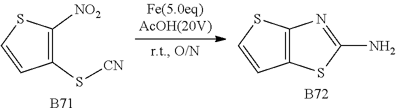 [SMILES: "B71" ; N#CSc1ccsc1[N+](=O)[O-] ; "B72" ; Nc1nc2sccc2s1]

[(2-nitrothiophen-3-yl)sulfanyl]formonitrile (15.5 g, 83.24 mmol) and AcOH (310 mL) were combined under a nitrogen atmosphere, followed by the addition Fe (23.2 g, 0.41 mmol) portionwise with stirring at 0° C. The reaction mixture was stirred for 16 h at room temperature, then quenched with a mixture of water and ice (300 mL). The reaction mixture was filtered to remove solids, and the filtrate concentrated under vacuum, pH adjusted to 8 with saturated aqueous Na₂CO₃, and extracted with 3×500 mL of ethyl acetate. The organic layers were combined, washed with saturated aqueous NaCl (1×1000 mL), filtered, and concentrated in vacuo to a residue. The residue was purified by silica gel column with ethyl acetate/petroleum ether to afford thieno[2,3-d][1,3]thiazol-2-amine (12.5 g, 96.12%) as a solid.

### Synthesis of Intermediate B73

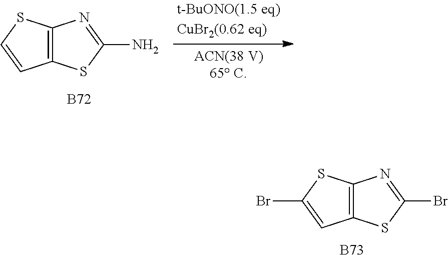

A solution of thieno[2,3-d][1,3]thiazol-2-amine (5.0 g, 32.00 mmol) in dry acetonitrile (125 mL) was treated dropwise with a solution of tBuNO₂ (4.9 g, 48.00 mmol) and CuBr₂ (4.4 g, 19.84 mmol) in dry acetonitrile (65 mL) under a nitrogen atmosphere. The reaction mixture was stirred for 10 min at 65° C., then quenched with HCl (6M, 150 mL), and extracted with ethoxyethane (3×400 mL). The organic layers were combined, washed with HCl (6 M, 1 x150 mL), dried over anhydrous sodium sulfate, filtered, and concentrated in vacuo to give a residue. The residue was purified by Flash-Prep-HPLC (Condition 1, Gradient 1) to afford 2,5-dibromothieno[2,3-d][1,3]thiazole (2.5 g, 25.34%) as a solid.

### Synthesis of Intermediate B74

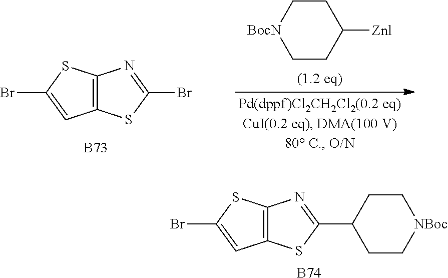

2,5-dibromothieno[2,3-d][1,3]thiazole (75.Oxl2 mg, 3.01 mmol), CuI (114.6 mg, 0.60 mmol), Pd(dppf)Cl₂·CH₂Cl₂ (440.5 mg, 0.60 mmol), and DMA (75.00 mL) were combined and the reaction vessel was evacuated and flushed three times with nitrogen. [1-(tert-butoxycarbonyl)piperidin-4-yl](iodo)zinc (B50, 1.3 g, 3.612 mmol) was added to the reaction mixture, and the reaction vessel evacuated and flushed three times with nitrogen. The reaction mixture was stirred overnight at 80° C., then quenched by the addition of water, filtered to remove solids, and extracted with ethyl acetate (3×100 mL). The organic layers were combined, washed with ½ saturated aqueous NaCl (3×150 mL) and saturated aqueous NaCl (1×150 mL), dried over anhydrous sodium sulfate, filtered, and concentrated in vacuo to give a residue. The residue was purified by Prep-TLC (PE: EA=10: 1) to afford tert-butyl 4-[5-bromothieno[2,3-d][1,3]thiazol-2-yl]piperidine-1-carboxylate (90 mg, 6.67%) as a solid. LCMS (ES, m/z): 403 [M+H]⁺.

### Synthesis of Intermediate B75

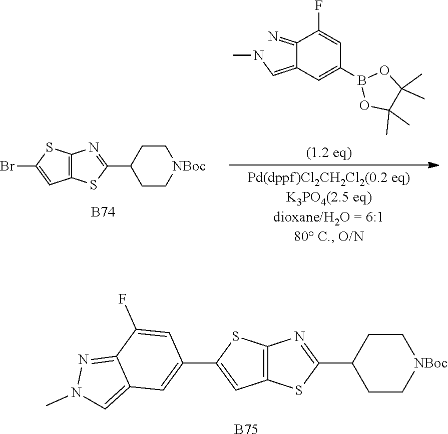

Tert-butyl 4-[5-bromothieno[2,3-d][1,3]thiazol-2-yl]piperidine-1-carboxylate (50.0 mg, 0.12 mmol), dioxane (5 mL), 7-fluoro-2-methyl-5-(4,4,5,5-tetramethyl-1,3,2-dioxaborolan-2-yl)indazole (41.0 mg, 0.15 mmol), K₃PO₄ (65.7 mg, 0.31 mmol), H₂O (1 mL), and Pd(dppf)Cl₂ CH₂Cl₂ (20.2 mg, 0.025 mmol) were combined. The reaction mixture was evacuated and flushed three times with nitrogen, then stirred overnight at 80° C. The reaction was quenched with water (20 mL) and extracted with ethyl acetate (3×30 mL). The organic layers were combined, washed with saturated aqueous NaCl (1×50 mL), dried over anhydrous sodium sulfate, filtered, and concentrated in vacuo to give a residue. The residue was purified by Prep-TLC (PE: EA=1:1) to afford tert-butyl 4-[5-(7-fluoro-2-methylindazol-5-yl)thieno[2,3-d][1,3]thiazol-2-yl]piperidine-1-carboxylate (20 mg, 34.14%) as a solid. LCMS (ES, m/z): 473 [M+H]⁺.

### Synthesis of Compound 124

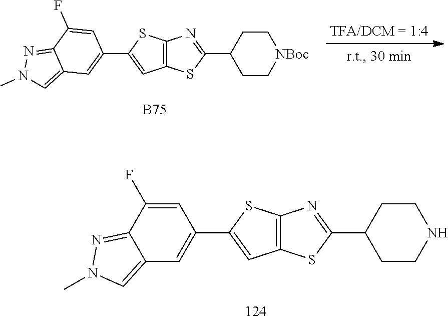 [SMILES: "B75" ; Cn1cc2cc(-c3cc4sc(C5CCN(C(=O)OC(C)(C)C)CC5)nc4s3)cc(F)c2n1 ; Cn1cc2cc(-c3cc4sc(C5CCNCC5)nc4s3)cc(F)c2n1 ; "124"]

Tert-butyl 4-[5-(7-fluoro-2-methylindazol-5-yl)thieno[2,3-d][1,3]thiazol-2-yl]piperidine-1-carboxylate (15.0 mg, 0.03 mmol), DCM (2 mL), and TFA (0.50 mL) were combined and stirred for 30 min at room temperature, then concentrated in vacuo to give a residue. The residue was purified by Prep-HPLC (Condition 1, Gradient 3) to afford 7-fluoro-2-methyl-5-[2-(piperidin-4-yl)thieno[2,3-d][1,3]thiazol-5-yl]indazole (6.3 mg, 53.29%) as a solid. LCMS (ES, m/z): 373 [M+H]+. ¹H NMR (400 MHz, DMSO-d₆) δ 8.52 (d, J=2.8 Hz, 1H), 7.89-7.81 (m, 2H), 7.49 (dd, J=12.9, 1.4 Hz, 1H), 4.22 (s, 3H), 3.20 ((ddt, 1H), 3.05 (dt, J=12.4, 3.4 Hz, 2H), 2.69-2.58 (m, 2H), 2.07-1.98 (m, 2H), 1.65 (qd, J=12.1, 4.0 Hz, 2H).

### Example 14: Synthesis of Compound 125

### Synthesis of Compound B76

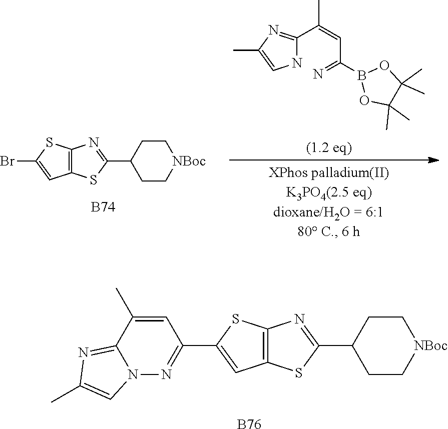

Tert-butyl 4-[5-bromothieno[2,3-d][1,3]thiazol-2-yl]piperidine-1-carboxylate (40.0 mg, 0.10 mmol), dioxane (3 mL), 2-methyl-6-(4,4,5,5-tetramethyl-1,3,2-dioxaborolan-2-yl)imidazo[1,2-b]pyridazine (30.8 mg, 0.12 mmol), K₃PO₄ (52.6 mg, 0.25 mmol), H₂O (0.50 mL), and XPhos palladium(II) biphenyl-2-amine chloride (11.7 mg, 0.015 mmol) were combined, and the reaction vessel was evacuated and flushed three times with nitrogen. The reaction mixture was stirred for 6 h at 80° C., then quenched with water (20 mL) and extracted with ethyl acetate (3×20 mL). The organic layers were combined, washed with saturated aqueous NaCl (1×50 mL), dried over anhydrous sodium sulfate, filtered, and concentrated in vacuo to give a residue. The residue was purified by Prep-TLC (PE: EA=1:1) to afford tert-butyl 4-(5-[2,8-dimethy-limidazo[1,2-b] pyridazin-6-yl]thieno[2,3-d][1,3]thiazol-2-yl)piperidine-1-carboxylate (30.0 mg, 64.4%) as a solid. LCMS (ES, m/z): 470 [M+H]⁺.

### Synthesis of Compound 125

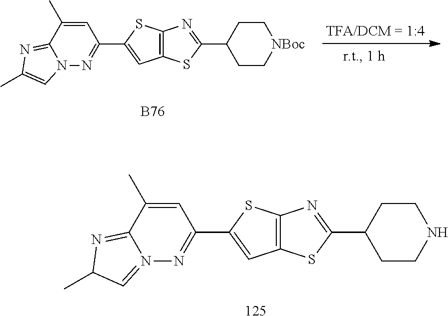

Tert-butyl 4-(5-[2,8-dimethylimidazo[1,2-b] pyridazin-6-yl]thieno[2,3-d][1,3]thiazol-2-yl)piperidine-1-carboxylate (25.00 mg), DCM (2 mL), and TFA (0.50 mL) were combined. The reaction mixture was stirred for 30 min at room temperature, then concentrated in vacuo to give a residue. The residue was purified by Prep-HPLC (Condition 1, Gradient 3) to afford 4-(5-[2,8-dimethylimidazo[1,2-b] pyridazin-6-yl]thieno [2,3-d][1,3]thiazol-2-yl) piperidine (12.0 mg, 50.8%) as a solid. LCMS (ES, m/z): 370[M+H]⁺. ¹H NMR (400 MHz, DMSO-d₆) δ 8.24 (s, 1H), 8.04 (d, J=1.0 Hz, 1H), 7.69 (d, J=1.3 Hz, 1H), 3.23-3.16 (m, 1H), 3.03 (dt, J=12.3, 3.3 Hz, 2H), 2.67-2.56 (m, 5H), 2.39 (s, 3H), 2.06-1.98 (m, 2H), 1.68-1.61 (dd, J=11.9, 3.9 Hz, 2H).

### Example 15: Synthesis of Compound 105

### Synthesis of Intermediate B78 and B79

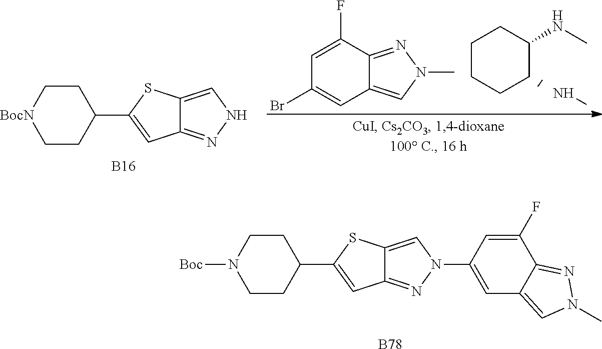

Tert-butyl 4-[2H-thieno[3,2-c]pyrazol-5-yl]piperidine-1-carboxylate (280.0 mg, 0.91 mmol, 1.0 equiv), 5-bromo-7-fluoro-2-methylindazole (250.3 mg, 1.09 mmol, 1.2 equiv), (1R,2S)—N1,N2-dimethylcyclohexane-1,2-diamine (51.8 mg, 0.36 mmol, 0.4 equiv), CuI (34.6 mg, 0.18 mmol, 0.2 equiv), Cs₂CO₃ (890.3 mg, 2.73 mmol, 3.0 equiv), and 1,4-dioxane (5.0 mL, 59.02 mmol, 64.8 equiv) under a nitrogen atmosphere and stirred for 16 h at 100° C. The reaction mixture was filtered, then concentrated in vacuo to give a residue. The residue was purified by Flash-Prep-HPLC (Condition 2, Gradient 1) to afford tert-butyl 4-[2-(7-fluoro-2-methylindazol-5-yl)thieno[3,2-c]pyrazol-5-yl]piperidine-1-carboxylate (45 mg) as a solid and tert-butyl 4-[1-(7-fluoro-2-methylindazol-5-yl)thieno[3,2-c] pyrazol-5-yl]piperidine-1-carboxylate (60 mg) as a solid. LCMS (ES, m/z): 456 [M+H]⁺.

### Synthesis of Compound 105

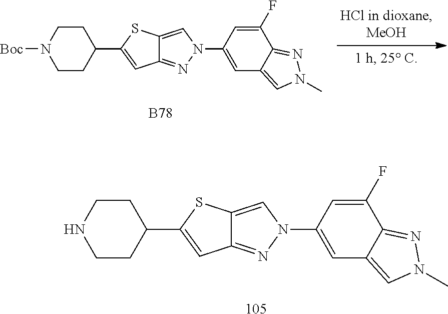 [SMILES: "B78" ; Cn1cc2cc(-n3cc4sc(C5CCN(C(=O)OC(C)(C)C)CC5)cc4n3)cc(F)c2n1 ; Cn1cc2cc(-n3cc4sc(C5CCNCC5)cc4n3)cc(F)c2n1 ; "105"]

Tert-butyl 4-[2-(7-fluoro-2-methylindazol-5-yl)thieno[3,2-c]pyrazol-5-yl]piperidine-1-carboxylate (45.0 mg) was combined with HCl (gas) in 1,4-dioxane (1.0 mL) in MeOH (1.0 mL) under a nitrogen atmosphere, and for 1 h at 25° C. The reaction mixture was concentrated in vacuo to give a residue. The residue was purified by Prep-HPLC (Condition 1, Gradient 4) to afford 7-fluoro-2-methyl-5-[5-(piperidin-4-yl)thieno[3,2-c]pyrazol-2-yl]indazole (16.1 mg) as a solid. LCMS (ES, m/z): 356 [M+H] *. ¹H NMR (400 MHz, DMSO-d₆) δ 8.63 (s, 1H), 8.52 (d, J=2.8 Hz, 1H), 7.97 (d, J=1.7 Hz, 1H), 7.71 (dd, J=12.7, 1.7 Hz, 1H), 6.97 (s, 1H), 4.23 (s, 3H), 3.23-3.34 (m, 2H), 2.96-3.01 (m, 1H), 2.68-2.76 (m, 2H), 1.95-2.01 (m, 2H), 1.52-1.73 (m, 2H).

### Example 16: Synthesis of Compound 135

### Synthesis of Intermediate B80

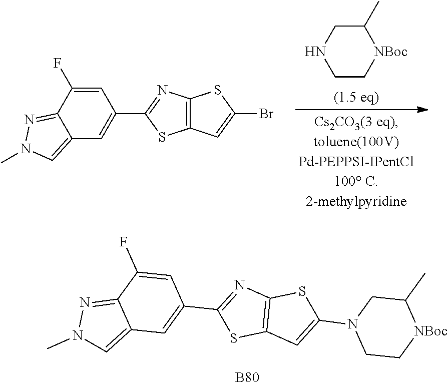

5-[5-bromothieno[2,3-d][1,3]thiazol-2-yl]-7-fluoro-2-methylindazole (50 mg, 0.14 mmol), tert-butyl 2-methylpiperazine-1-carboxylate (40.79 mg, 0.20 mmol), Pd-PEPPSI-IPentCl 2-methylpyridine (o-picoline, 11.42 mg, 0.01 mmol), and Cs₂CO₃ (132.72 mg, 0.41 mmol) were combined in a sealed tube in toluene (3 mL) under a nitrogen atmosphere and stirred for 10 h at 100° C. The reaction mixture was extracted with ethyl acetate (3×10 mL). The organic layers were combined, washed with a saturated NaCl solution (1×10 mL), dried over anhydrous sodium sulfate, and concentrated in vacuo to give a residue. The residue was purified by silica gel column chromatography with ethyl acetate/petroleum ether (1:4) to afford tert-butyl 4-[2-(7-fluoro-2-methylindazol-5-yl)thieno[2,3-d][1,3]thiazol-5-yl]-2-methylpiperazine-1-carboxylate (40.00 mg, 60.42%) as a solid. LCMS (ES, m/z): 488 [M+H]⁺.

### Synthesis of Compound 135

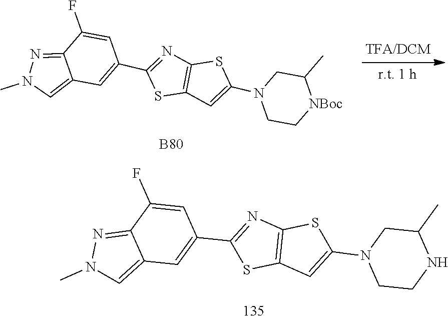

Tert-butyl 4-[2-(7-fluoro-2-methylindazol-5-yl)thieno[2,3-d][1,3]thiazol-5-yl]-2-methylpiperazine-1-carboxylate (40.00 mg, 0.08 mmol) was combined with a mixture of TFA and DCM (5 mL). The reaction mixture was stirred for 1 h at room temperature, then concentrated in vacuo to a residue. The residue was purified by Prep-HPLC (Condition 2, Gradient 4) to afford 7-fluoro-2-methyl-5-[5-(3-methylpiperazin-1-yl)thieno[2,3-d][1,3]thiazol-2-yl]indazole (19.10 mg, 60.09%) as a solid. LCMS (ES, m/z):388 [M+H] *.

¹H NMR (400 MHz, DMSO-d₆) δ 8.57 (d, J=2.8 Hz, 1H), 8.09 (d, J=1.3 Hz, 1H), 7.59 (dd, J=12.7, 1.4 Hz, 1H), 6.50 (s, 1H), 4.23 (s, 3H), 3.39 (td, J=7.6, 3.1 Hz, 2H), 3.02-2.94 (m, 1H), 2.88-2.71 (m, 3H), 2.43 (t, J=10.8 Hz, 1H), 1.04 (d, J=6.3 Hz, 3H).

### Example 17: Synthesis of Compound 128

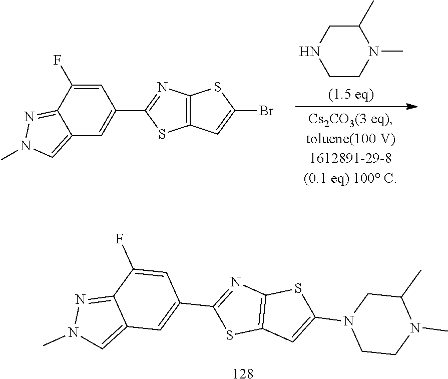

5-[5-bromothieno[2,3-d][1,3]thiazol-2-yl]-7-fluoro-2-methylindazole (50.00 mg, 0.14 mmol), 1,2-dimethylpiperazine (23.26 mg, 0.20 mmol), Pd-PEPPSI-IPentCl 2-methylpyridine (o-picoline (11.42 mg, 0.01 mmol), Cs₂CO₃ (132.72 mg, 0.41 mmol) and toluene were combined in a sealed tube under a nitrogen atmosphere and stirred for 10 h at 100° C. The reaction mixture was extracted with ethyl acetate (3×10 mL). The organic layers were combined, washed with saturated NaCl (1×10 mL), dried over anhydrous sodium sulfate, and concentrated in vacuo to give a residue. The residue was purified by silica gel column chromatography with ethyl acetate/petroleum ether (1:4), followed by Prep-HPLC (Condition 4, Gradient 1) to afford 5-[5-(3,4-dimethylpiperazin-1-yl)thieno[2,3-d][1,3]thiazol-2-yl]-7-fluoro-2-methylindazole (11.70 mg, 21.46%) as a solid. LCMS (ES, m/z):402 [M+H]⁺. ¹H NMR (400 MHz, DMSO-d₆) δ 8.57 (d, J=2.8 Hz, 1H), 8.10 (d, J=1.4 Hz, 1H), 7.59 (dd, J=12.6, 1.4 Hz, 1H), 6.52 (s, 1H), 4.23 (s, 3H), 3.41 (t, J=12.4 Hz, 2H), 2.97 (td, J=11.5, 3.1 Hz, 1H), 2.84 (d, J=11.5 Hz, 1H), 2.61 (t, J=10.8 Hz, 1H), 2.33-2.25 (m, 1H), 2.23 (s, 3H), 2.19 (d, J=6.8 Hz, 1H), 1.07 (d, J=6.2 Hz, 3H).

### Example 18: Synthesis of Compound 136

### Synthesis of Intermediate B81

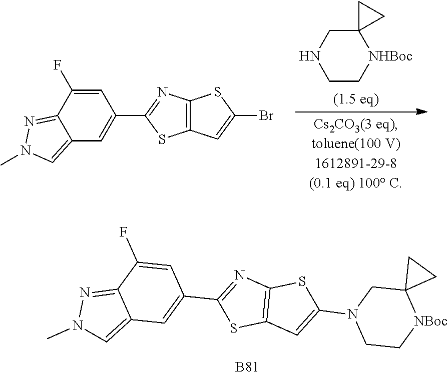

5-[5-bromothieno[2,3-d][1,3]thiazol-2-yl]-7-fluoro-2-methylindazole (50.00 mg, 0.14 mmol), tert-butyl 4,7-diazaspiro[2.5]octane-4-carboxylate (43.24 mg, 0.20 mmol), Pd-PEPPSI-IPentCl 2-methylpyridine (o-picoline) (11.42 mg, 0.01 mmol), Cs₂CO₃ (132.72 mg, 0.41 mmol), and toluene (3 mL) were combined in a sealed tube under a nitrogen atmosphere and stirred for 10 h at 100° C. The reaction mixture was extracted with ethyl acetate (3×10 mL). The organic layers were combined, washed with a saturated NaCl solution (1×10 mL), dried over anhydrous sodium sulfate, and concentrated in vacuo to give a residue. The residue was purified by silica gel column chromatography with ethyl acetate/petroleum ether (1:4) to afford tert-butyl 7-[2-(7-fluoro-2-methylindazol-5-yl)thieno[2,3-d][1,3]thiazol-5-yl]-4,7-diazaspiro[2.5]octane-4-carboxylate (37.00 mg, 54.54%) as a solid. LCMS (ES, m/z):500 [M+H]⁺.

### Synthesis of Compound 136

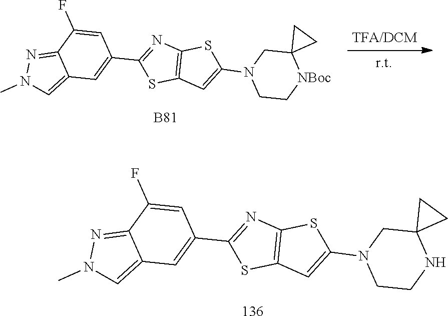

Tert-butyl 7-[2-(7-fluoro-2-methylindazol-5-yl)thieno[2,3-d][1,3]thiazol-5-yl]-4,7-diazaspiro[2.5]octane-4-carboxylate (37.00 mg, 0.07 mmol) was added to a mixture of TFA and DCM (5 mL). The reaction mixture was stirred for 1 h at room temperature, then concentrated in vacuo to give a residue. The resiude was purified by Prep-HPLC (Condition 2, Gradient 2) to afford 5-(5-[4,7-diazaspiro[2.5]octan-7-yl]thieno[2,3-d][1,3]thiazol-2-yl)-7-fluoro-2-methylindazole (6.60 mg, 22.31%) as a solid. LCMS (ES, m/z):400 [M+H]⁺. ¹H NMR (400 MHz, DMSO-d₆) δ 8.57 (d, J=2.7 Hz, 1H), 8.09 (d, J=1.3 Hz, 1H), 7.59 (dd, J=12.6, 1.4 Hz, 1H), 6.47 (s, 1H), 4.23 (s, 3H), 3.13 (dd, J=6.0, 4.2 Hz, 2H), 3.01 (s, 2H), 2.92 (t, J=5.1 Hz, 2H), 2.45 (brs, 1H), 0.54 (dt, J=9.6, 2.1 Hz, 4H).

### Example 19: Synthesis of Compound 129

### Synthesis of Compound 129

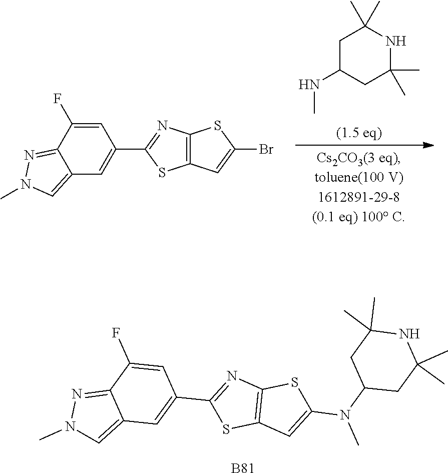

5-[5-bromothieno[2,3-d][1,3]thiazol-2-yl]-7-fluoro-2-methylindazole (50.00 mg, 0.14 mmol), N,2,2,6,6-pentamethylpiperidin-4-amine (34.69 mg, 0.20 mmol), Pd-PEPPSI-IPentCl 2-methylpyridine (o-picoline) (11.42 mg, 0.01 mmol), Cs₂CO₃ (132.72 mg, 0.41 mmol), and toluene (3 ml) were combined in a sealed tube under a nitrogen atmosphere. The reaction mixture was stirred for 10 h at 100° C., diluted with water, and extracted with ethyl acetate (3×10 mL). The organic layers were combined, washed with of a saturated NaCl solution (1×10 mL), dried over anhydrous sodium sulfate, and concentrated in vacuo to give a residue. The residue was purified by silica gel column chromatography with ethyl acetate/petroleum ether (1:4), followed by Prep-HPLC (Condition 5, Gradient 1) to afford N-[2-(7-fluoro-2-methylindazol-5-yl)thieno[2,3-d][1,3]thiazol-5-yl]-N,2,2,6,6-pentamethylpiperidin-4-amine (2 mg, 3.22%) as a solid. LCMS (ES, m/z):458 [M+H]⁺. ¹H NMR (400 MHz, DMSO-d₆) δ 8.56 (d, J=2.8 Hz, 1H), 8.06 (d, J=1.3 Hz, 1H), 7.58 (dd, J=12.6, 1.4 Hz, 1H), 6.32 (s, 1H), 4.22 (s, 3H), 3.78 (t, J=12.5 Hz, 1H), 2.81 (s, 3H), 1.64 (d, J=11.6 Hz, 2H), 1.40 (t, J=12.4 Hz, 2H), 1.25 (s, 6H), 1.11 (s, 6H).

### Example 20: Synthesis of Compound 137

### Synthesis of Intermediate B82

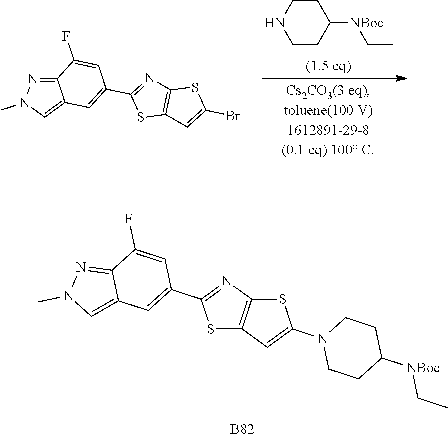 [SMILES: Cn1cc2cc(-c3nc4sc(Br)cc4s3)cc(F)c2n1 ; CCN(C(=O)OC(C)(C)C)C1CCNCC1 ; "B82" ; CCN(C(=O)OC(C)(C)C)C1CCN(c2cc3sc(-c4cc(F)c5nn(C)cc5c4)nc3s2)CC1]

5-[5-bromothieno[2,3-d][1,3]thiazol-2-yl]-7-fluoro-2-methylindazole (50.00 mg, 0.14 mmol), tert-butyl N-ethyl-N-(piperidin-4-yl)carbamate (46.51 mg, 0.20 mmol), Pd-PEPPSI-IPentCl 2-methylpyridine (o-picoline) (11.42 mg, 0.01 mmol), Cs₂CO₃ (132.72 mg, 0.41 mmol), and toluene (3 mL) were combined in a sealed tube under a nitrogen atmosphere and stirred for 10 h at 100° C. The reaction mixture was extracted with ethyl acetate (3×10 mL). The organic layers were combined, washed with of a saturated NaCl solution (1×10 mL), dried over anhydrous sodium sulfate, and concentrated in vacuo to give a residue. The residue was purified by silica gel column chromatography with ethyl acetate/petroleum ether (1:4) to afford tert-butyl N-ethyl-N-[1-[2-(7-fluoro-2-methylindazol-5-yl)thieno[2,3-d][1,3]thiazol-5-yl]piperidin-4-yl]carbamate (40.00 mg, 57.13%) as a solid. LCMS (ES, m/z): 516 [M+H]⁺.

### Synthesis of Compound 137

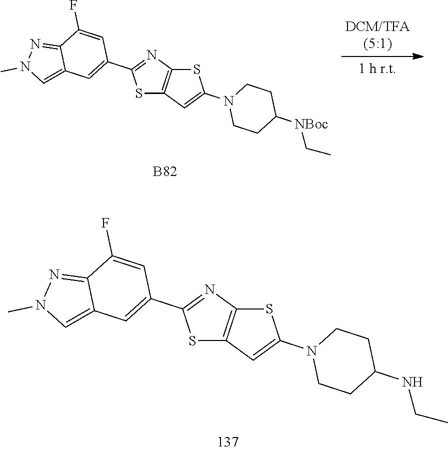

Tert-butyl N-ethyl-N-[1-[2-(7-fluoro-2-methylindazol-5-yl)thieno[2,3-d][1,3]thiazol-5-yl]piperidin-4-yl]carbamate (40.00 mg, 0.08 mmol) was added to a mixture of TFA and DCM (5 mL). The reaction mixture was stirred for 1 h at room temperature and concentrated in vacuo to give a residue. The residue was purified by Prep-HPLC (Condition 2, Gradient 5) to afford N-ethyl-1-[2-(7-fluoro-2-methylindazol-5-yl)thieno[2,3-d][1,3]thiazol-5-yl]piperidin-4-amine (8.90 mg, 27.61%) as a solid. LCMS (ES, m/z): 416 [M+H]⁺. ¹H NMR (400 MHz, DMSO-d₆) δ 8.57 (d, J=2.8 Hz, 1H), 8.08 (d, J=1.3 Hz, 1H), 7.59 (dd, J=12.6, 1.4 Hz, 1H), 6.49 (s, 1H), 4.23 (s, 3H), 3.57-3.49 (m, 2H), 2.94 (td, J=11.5, 3.0 Hz, 2H), 2.60 (q, J=7.2 Hz, 3H), 1.96-1.87 (m, 2H), 1.40 (s, 2H), 1.03 (t, J=7.1 Hz, 3H).

### Example 21: Synthesis of Compound 131

### Synthesis of Intermediate B83

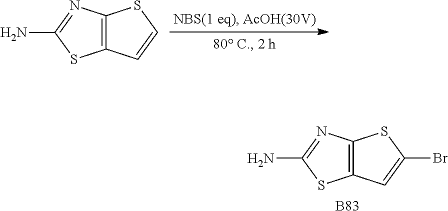

Thieno[2,3-d][1,3]thiazol-2-amine (30.00 g, 192.04 mmol), AcOH (900 ml), and NBS (34.18 g, 192.04 mmol) were combined and stirred for 2 h at 80° C. The reaction mixture was pH adjusted to 8 with Na₂CO₃, extracted with ethyl acetate (3×500 mL). The organic layers were combined, washed with a saturated NaCl solution (1×500 mL), dried over anhydrous sodium sulfate, and concentrated in vacuo to a residue. The residue was purified by silica gel column chromatography with ethyl acetate/petroleum ether (1:10) to afford 5-bromothieno[2,3-d][1,3]thiazol-2-amine (5.90 g, 13.07%) as a solid. LCMS (ES, m/z):235 [M+H]⁺.

### Synthesis of Intermediate B84

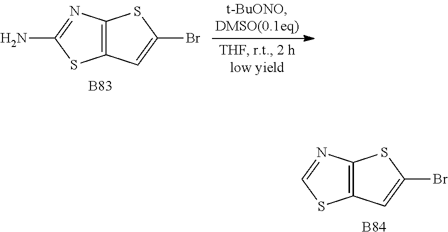

5-bromothieno[2,3-d][1,3]thiazol-2-amine (5.90 g, 25.09 mmol), THF (120 mL), t-BuNO₂ (3.88 g, 37.64 mmol), and DMSO (196.06 mg, 2.51 mmol) were combined and stirred for 2 h at room temperature, then concentrated in vacuo to a residue. The residue was purified by silica gel column chromatography with ethyl acetate/petroleum ether (1:10) to afford 5-bromothieno[2,3-d][1,3]thiazole (2.35 g, 42.55%) as a solid. LCMS (ES, m/z):220 [M+H]⁺.

### Synthesis of Intermediate B85

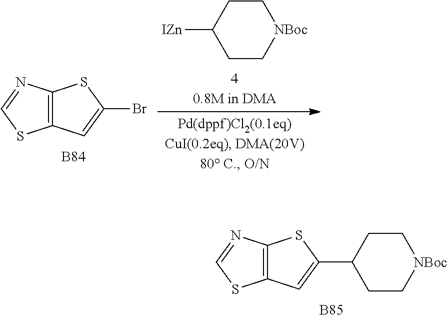

5-bromothieno[2,3-d][1,3]thiazole (1.35 g, 6.13 mmol), DMA (40 ml), CuI (0.23 g, 1.23 mmol), Pd(dppf)Cl₂ (0.50 g, 0.61 mmol), and [1-(tert-butoxycarbonyl) piperidin-4-yl](iodo)zinc (4.62 g, 12.27 mmol) were combined under a nitrogen atmosphere. The reaction mixture was stirred for 10 h at 110° C., then quenched with a mixture of water and ice (20 mL) and extracted with ethyl acetate (3×50 mL). The organic layers were combined, washed with a saturated NaCl solution (1×50 mL), dried over anhydrous sodium sulfate, and concentrated in vacuo to a residue. The residue was purified by silica gel column chromatography with ethyl acetate/petroleum ether (1:4) to afford tert-butyl 4-[thieno[2,3-d][1,3]thiazol-5-yl]piperidine-1-carboxylate (690 mg, 34.67%) as a solid. LCMS (ES, m/z):325 [M+H]⁺.

### Synthesis of Intermediate B86

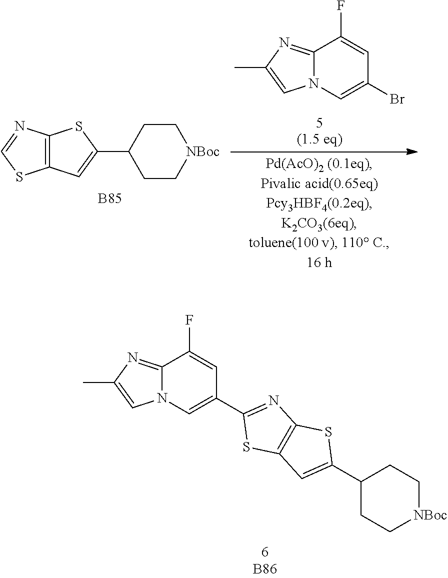 [SMILES: "B85" ; CC(C)(C)OC(=O)N1CCC(c2cc3scnc3s2)CC1 ; Cc1cn2cc(-c3nc4sc(C5CCN(C(=O)OC(C)(C)C)CC5)cc4s3)cc(F)c2n1 ; Cc1cn2cc(Br)cc(F)c2n1]

Tert-butyl 4-[thieno[2,3-d][1,3]thiazol-5-yl]piperidine-1-carboxylate (50.00 mg, 0.15 mmol), 6-bromo-8-fluoro-2-methylimidazo[1,2-a]pyridine (52.95 mg, 0.23 mmol), Pd(AcO)₂ (3.46 mg, 0.02 mmol), Pivalic acid (10.23 mg, 0.10 mmol), PCy₃HBF₄ (11.35 mg, 0.03 mmol), K₂CO₃ (127.79 mg, 0.93 mmol), and toluene (3 mL) were combined under a nitrogen atmosphere. The reaction mixture was stirred for 16 h at 110° C., then extracted with ethyl acetate (3×10 mL). The organic layers were combined, washed with a saturated NaCl solution (1×10 mL), dried over anhydrous sodium sulfate, and concentrated in vacuo to give a residue. The residue was purified by silica gel column chromatography with ethyl acetate/petroleum ether (1:4) to afford tert-butyl 4-(2-[8-fluoro-2-methylimidazo[1,2-a]pyridin-6-yl]thieno[2,3-d][1,3]thiazol-5-yl)piperidine-1-carboxylate (35 mg, 48.06%) as a solid. LCMS (ES, m/z):473 [M+H]⁺.

### Synthesis of Compound 130

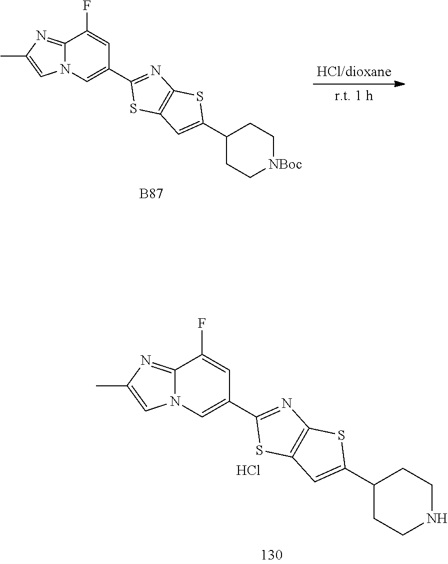 [SMILES: "B87" ; Cc1cn2cc(-c3nc4sc(C5CCN(C(=O)OC(C)(C)C)CC5)cc4s3)cc(F)c2n1 ; Cc1cn2cc(-c3nc4sc(C5CCNCC5)cc4s3)cc(F)c2n1 ; Cl]

Tert-butyl 4-(2-[8-fluoro-2-methylimidazo[1,2-a]pyridin-6-yl]thieno[2,3-d][1,3]thiazol-5-yl)piperidine-1-carboxylate (35 mg) and HCl (gas) in 1,4-dioxane (5 mL) were stirred for 1 h at room temperature. The reaction mixture was concentrated in vacuo to give a residue. The residue was purified by Prep-HPLC (Condition 6, Gradient 1) to afford 4-(2-[8-fluoro-2-methylimidazo[1,2-a]pyridin-6-yl]thieno[2,3-d][1,3]thiazol-5-yl)piperidine hydrochloride (14.70 mg, 48.54%) as a solid. LCMS (ES, m/z):373 [M+H]⁺. ¹H NMR (400 MHz, DMSO-d₆) δ 9.24 (t, J=1.6 Hz, 1H), 8.76 (s, 1H), 8.54 (s, 1H), 7.98 (s, 1H), 7.77 (d, J=11.3 Hz, 1H), 7.40 (d, J=1.0 Hz, 1H), 3.39 (d, J=12.5 Hz, 2H), 3.29 (d, J=11.4 Hz, 1H), 3.08 (d, J=12.2 Hz, 2H), 2.42 (s, 3H), 2.19 (d, J=13.6 Hz, 2H), 1.88 (s, 1H), 1.84 (d, J=11.3 Hz, 1H).

### Example 21: Synthesis of Compound 131

### Synthesis of Intermediate B88

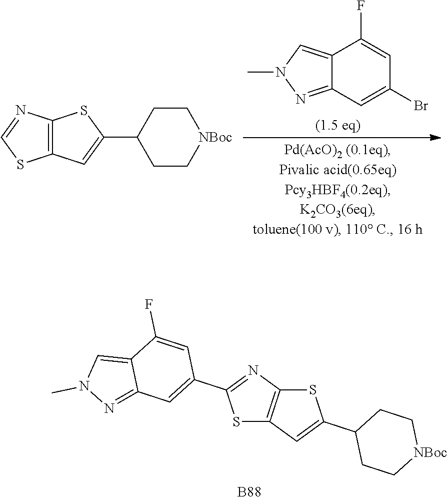 [SMILES: Cn1cc2c(F)cc(Br)cc2n1 ; CC(C)(C)OC(=O)N1CCC(c2cc3scnc3s2)CC1 ; "B88" ; Cn1cc2c(F)cc(-c3nc4sc(C5CCN(C(=O)OC(C)(C)C)CC5)cc4s3)cc2n1]

Into a 8-mL sealed tube purged and maintained with an inert atmosphere of nitrogen, was placed tert-butyl 4-[thieno[2,3-d][1,3]thiazol-5-yl]piperidine-1-carboxylate (B85, 50.00 mg, 0.15 mmol, 1.00 equiv), 6-bromo-4-fluoro-2-methylindazole (52.95 mg, 0.23 mmol, 1.50 equiv), Pd(AcO)₂ (3.46 mg, 0.02 mmol, 0.10 equiv), pivalic acid (10.23 mg, 0.10 mmol, 0.65 equiv), PCy₃HBF₄ (11.35 mg, 0.03 mmol, 0.20 equiv), K₂CO₃ (127.79 mg, 0.92 mmol, 6.00 equiv), Toluene (3.00 mL). The resulting solution was stirred for 16 hr at 110° C. The resulting solution was extracted with 3×10 mL of ethyl acetate and the organic layers combined. The resulting mixture was washed with 1 x10 ml of sat. NaCl. The mixture was dried over anhydrous sodium sulfate and concentrated under vacuum. The residue was applied onto a silica gel column with ethyl acetate/petroleum ether (1:4). This resulted in 35.00 mg (48.06%) of tert-butyl 4-[2-(4-fluoro-2-methylindazol-6-yl)thieno[2,3-d][1,3]thiazol-5-yl]piperidine-1-carboxylate as a solid. LCMS (ES, m/z):473 [M+H]⁺.

### Synthesis of Compound 131

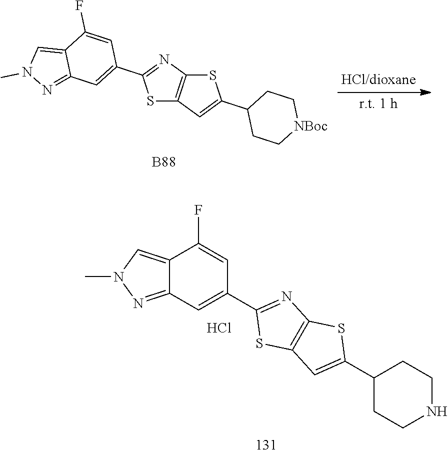 [SMILES: "B88" ; Cn1cc2c(F)cc(-c3nc4sc(C5CCN(C(=O)OC(C)(C)C)CC5)cc4s3)cc2n1 ; Cn1cc2c(F)cc(-c3nc4sc(C5CCNCC5)cc4s3)cc2n1 ; Cl ; "131"]

Tert-butyl 4-[2-(4-fluoro-2-methylindazol-6-yl)thieno [2,3-d][1,3]thiazol-5-yl]piperidine-1-carboxylate (35.00 mg, 0.07 mmol) and HCl (gas) in 1,4-dioxane (5 mL, 87.59 mmol) were combined and stirred for 1 h at room temperature. The reaction mixture was concentrated in vacuo in a residue. The residue was purified by Prep-HPLC (Condition 6, Gradient 1) to afford 4-fluoro-2-methyl-6-[5-(piperidin-4-yl)thieno[2,3-d][1,3]thiazol-2-yl]indazole (9.50 mg, 34.44%) as a solid. LCMS (ES, m/z):373 [M+H]⁺. ¹H NMR (400 MHz, DMSO-d₆) δ 8.90 (d, J=11.3 Hz, 1H), 8.65 (s, 2H), 8.08 (s, 1H), 7.43 (dd, J=11.4, 1.2 Hz, 1H), 7.38 (d, J=1.0 Hz, 1H), 4.23 (s, 3H), 3.42-3.35 (m, 3H), 3.1-3.0 (m, 2H), 2.23-2.14 (m, 2H), 1.96-1.81 (m, 2H).

### Example 22: Synthesis of Compound 132

### Synthesis of Intermediate B89

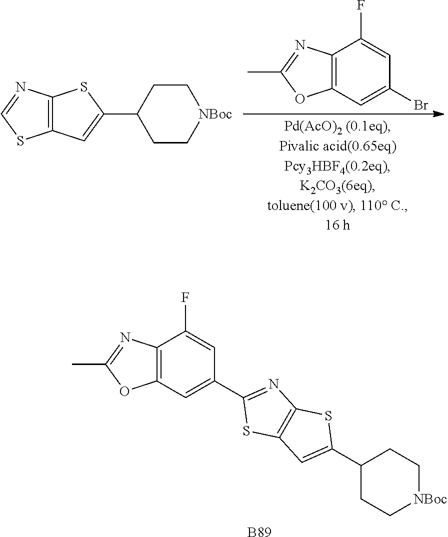 [SMILES: CC(C)(C)OC(=O)N1CCC(c2cc3scnc3s2)CC1 ; Cc1nc2c(F)cc(Br)cc2o1 ; "B89" ; Cc1nc2c(F)cc(-c3nc4sc(C5CCN(C(=O)OC(C)(C)C)CC5)cc4s3)cc2o1]

Tert-butyl 4-[thieno[2,3-d][1,3]thiazol-5-yl]piperidine-1-carboxylate (50.00 mg, 0.15 mmol), 6-bromo-4-fluoro-2-methyl-1,3-benzoxazole (B85, 53.17 mg, 0.23 mmol), Pd(AcO)₂ (3.46 mg, 0.02 mmol), Pivalic acid (10.23 mg, 0.1 mmol), PCy₃HBF₄ (11.35 mg, 0.03 mmol), and K₂CO₃ (127.79 mg, 0.92 mmol) were combined in toluene (5 mL). The reaction mixture was stirred for 16 h at 110° C., then extracted with ethyl acetate (3×10 mL). The organic layers were combined, washed with a saturated NaCl solution (1×10 mL), dried over anhydrous sodium sulfate, and concentrated in vacuo to give a residue. The residue was purified by silica gel column chromatography with ethyl acetate/petroleum ether (1:4) to afford tert-butyl 4-[2-(4-fluoro-2-methyl-1,3-benzoxazol-6-yl)thieno[2,3-d][1,3]thiazol-5-yl]piperidine-1-carboxylate (42 mg, 57.55%) as a solid. LCMS (ES, m/z): 474 [M+H]⁺.

### Synthesis of Compound 132

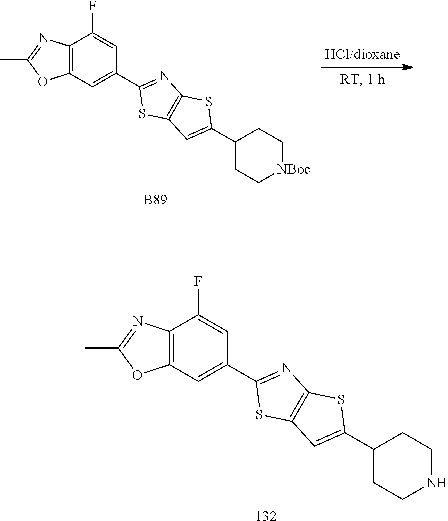 [SMILES: "B89" ; Cc1nc2c(F)cc(-c3nc4sc(C5CCN(C(=O)OC(C)(C)C)CC5)cc4s3)cc2o1 ; Cc1nc2c(F)cc(-c3nc4sc(C5CCNCC5)cc4s3)cc2o1 ; "132"]

Tert-butyl 4-[2-(4-fluoro-2-methyl-1,3-benzoxazol-6-yl)thieno[2,3-d][1,3]thiazol-5-yl]piperidine-1-carboxylate (42 mg, 0.09 mmol) and HCl (gas) in 1,4-dioxane (5 mL, 87.59 mmol) were combined and stirred for 1 h at room temperature. The reaction mixture was concentrated in vacuo to give a residue. The residue was purified by Prep-HPLC (Condition 5, Gradient 2) to afford 4-fluoro-2-methyl-6-[5-(piperidin-4-yl)thieno[2,3-d][1,3]thiazol-2-yl]-1,3-benzoxazole (4.30 mg, 12.98%) as a solid. LCMS (ES, m/z):374 [M+H]⁺. ¹H NMR (400 MHz, DMSO-d₆) δ 8.18 (d, J=1.4 Hz, 1H), 7.85 (dd, J=10.9, 1.4 Hz, 1H), 7.35 (d, J=1.0 Hz, 1H), 3.18-3.05 (m, 3H), 2.79-2.69 (m, 2H), 2.69 (s, 3H), 2.02 (d, J=12.8 Hz, 2H), 1.64 (qd, J=12.3, 3.9 Hz, 2H).

### Example 23: Synthesis of Compound 126

### Synthesis of Intermediate B90

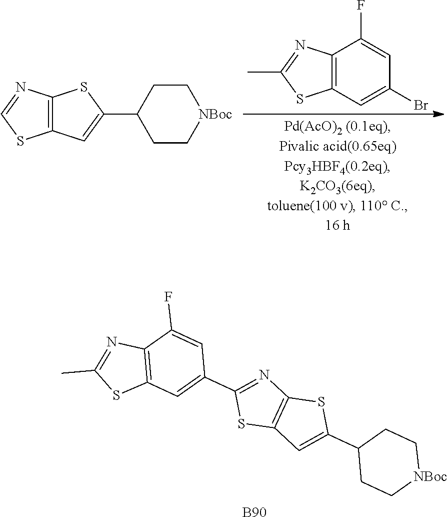 [SMILES: CC(C)(C)OC(=O)N1CCC(c2cc3scnc3s2)CC1 ; Cc1nc2c(F)cc(Br)cc2s1 ; "B90" ; Cc1nc2c(F)cc(-c3nc4sc(C5CCN(C(=O)OC(C)(C)C)CC5)cc4s3)cc2s1]

Tert-butyl 4-[thieno[2,3-d][1,3]thiazol-5-yl]piperidine-1-carboxylate (B85, 50.00 mg, 0.15 mmol), 6-bromo-4-fluoro-2-methyl-1,3-benzothiazole (56.89 mg, 0.23 mmol), Pd(AcO)₂ (3.46 mg, 0.015 mmol), Pivalic acid (10.23 mg, 0.1 mmol), PCy₃HBF₄ (11.35 mg, 0.03 mmol), and K₂CO₃ (127.79 mg, 0.93 mmol) were combined in toluene. The reaction mixture was stirred for 16 h at 110° C., extracted with ethyl acetate (3×10 mL). The organic layers were combined, washed with a saturated NaCl solution (1×10 mL), dried over anhydrous sodium sulfate, and concentrated in vacuo to give a residue. The residue was purified by silica gel column chromatography with ethyl acetate/petroleum ether (1:4) to afford tert-butyl 4-[2-(4-fluoro-2-methyl-1,3-benzothiazol-6-yl)thieno[2,3-d][1,3]thiazol-5-yl]piperidine-1-carboxylate (40 mg, 53.01%) as a solid. LCMS (ES, m/z):490 [M+H]⁺.

### Synthesis of Compound 126

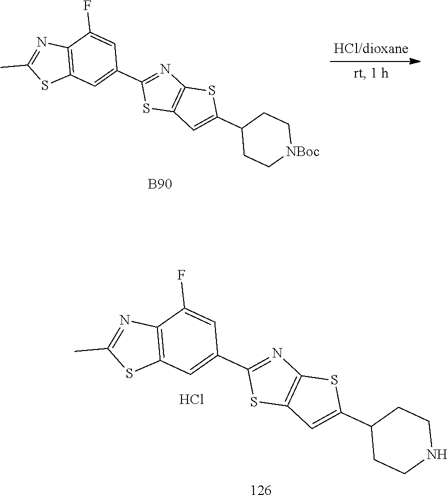 [SMILES: "B90" ; Cc1nc2c(F)cc(-c3nc4sc(C5CCN(C(=O)OC(C)(C)C)CC5)cc4s3)cc2s1 ; Cc1nc2c(F)cc(-c3nc4sc(C5CCNCC5)cc4s3)cc2s1 ; Cl ; "126"]

Tert-butyl 4-[2-(4-fluoro-2-methyl-1,3-benzothiazol-6-yl) thieno[2,3-d][1,3]thiazol-5-yl]piperidine-1-carboxylate (40.00 mg, 0.08 mmol) was combined with HCl(gas) in 1,4-dioxane (5.00 mL, 87.59 mmol). The reaction mixture was stirred for 1 hr at room temperature, then concentrated in vacuo to give a residue. The residue was purified by Prep-HPLC (Condition 6, Gradient 1) to afford 4-fluoro-2-methyl-6-[5-(piperidin-4-yl)thieno[2,3-d][1,3]thiazol-2-yl]-1,3-benzothiazole hydrochloride (22.10 mg, 63.51%) as a solid. LCMS (ES, m/z):390 [M+H]⁺. ¹H NMR (400 MHz, DMSO-d₆) δ 8.89 (s, 1H), 8.66 (d, J=10.8 Hz, 1H), 8.59 (d, J=1.6 Hz, 1H), 7.93 (dd, J=11.5, 1.6 Hz, 1H), 7.40 (d, J=1.0 Hz, 1H), 3.38 (d, J=12.3 Hz, 2H), 3.32 (s, 1H), 3.04 (q, J=12.0 Hz, 2H), 2.87 (s, 3H), 2.24-2.15 (m, 2H), 1.96-1.81 (m, 2H).

### Example 24: Synthesis of Compound 138

### Synthesis of Intermediate B91

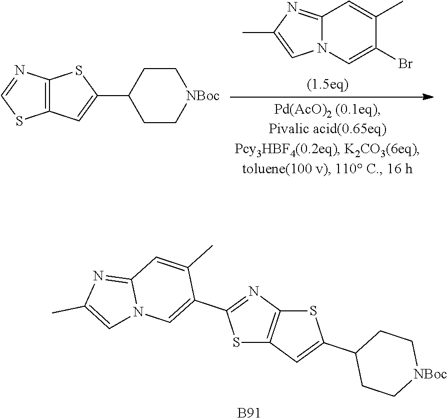 [SMILES: Cc1cn2cc(Br)c(C)cc2n1 ; CC(C)(C)OC(=O)N1CCC(c2cc3scnc3s2)CC1 ; "B91" ; Cc1cn2cc(-c3nc4sc(C5CCN(C(=O)OC(C)(C)C)CC5)cc4s3)c(C)cc2n1]

Tert-butyl 4-[thieno[2,3-d][1,3]thiazol-5-yl]piperidine-1-carboxylate (B85, 50.00 mg, 0.15 mmol), 6-bromo-2,7-dimethylimidazo[1,2-a]pyridine (52.03 mg, 0.23 mmol), Pd(AcO)₂ (3.46 mg, 0.02 mmol), Pivalic acid (10.23 mg, 0.10 mmol), PCy3·HBF4 (11.35 mg, 0.03 mmol), K₂CO₃ (127.79 mg, 0.92 mmol), and toluene (3 mL) were combined under a nitrogen atmosphere. The reaction mixture was stirred for 16 h at 110° C., extracted with ethyl acetate (3×10 mL). The organic layers were combined, washed with a saturated NaCl solution (1×10 mL), dried over anhydrous sodium sulfate, and concentrated in vacuo to give a residue. The residue was purified by silica gel column chromatography with ethyl acetate/petroleum ether (1:4) to afford tert-butyl 4-(2-[2,7-dimethylimidazo[1,2-a]pyridin-6-yl]thieno[2,3-d][1,3]thiazol-5-yl)piperidine-1-carboxylate (20.0 mg, 27.69%) as a solid. LCMS (ES, m/z): 469 [M+H]⁺.

### Synthesis of Compound 138

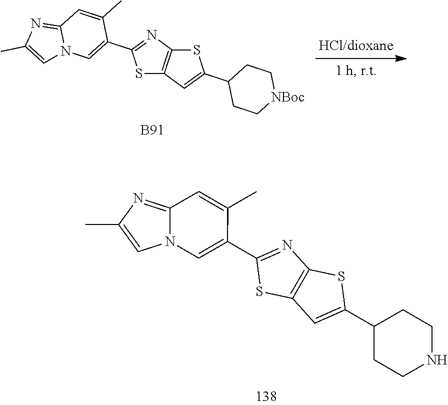 [SMILES: "B91" ; Cc1cn2cc(-c3nc4sc(C5CCN(C(=O)OC(C)(C)C)CC5)cc4s3)c(C)cc2n1 ; Cc1cn2cc(-c3nc4sc(C5CCNCC5)cc4s3)c(C)cc2n1]

Tert-butyl 4-(2-[2,7-dimethylimidazo[1,2-a]pyridin-6-yl]thieno[2,3-d][1,3]thiazol-5-yl)piperidine-1-carboxylate (20 mg, 0.04 mmol) was combined with HCl (gas) in 1,4-dioxane (5 mL) and stirred for 1 h at room temperature. The reaction mixture was concentrated in vacuo to give a residue. The residue was purified by Prep-HPLC (Condition 2, Gradient 4) to afford 4-(2-[2,7-dimethylimidazo[1,2-a]pyridin-6-yl]thieno[2,3-d][1,3]thiazol-5-yl)piperidine (1.00 mg, 6.36%) as a solid. LCMS (ES, m/z):369 [M+H]⁺. ¹H NMR (400 MHz, Methanol-d₄) δ 8.75 (s, 1H), 7.62 (s, 1H), 7.39 (s, 1H), 7.21 (d, J=0.9 Hz, 1H), 3.22 (d, J=3.3 Hz, 3H), 3.23-3.09 (m, 2H), 2.82 (td, J=12.5, 2.6 Hz, 3H), 2.60 (d, J=1.2 Hz, 3H), 2.17-2.09 (m, 2H), 1.78 (qd, J=12.3, 3.9 Hz, 2H).

### Example 25: Synthesis of Compound 133

### Synthesis of Intermediate B92

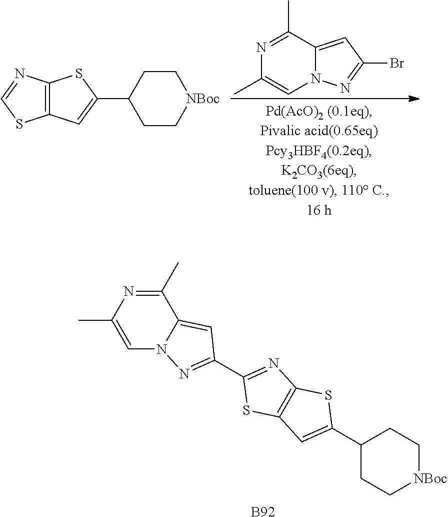

Tert-butyl 4-[thieno[2,3-d][1,3]thiazol-5-yl]piperidine-1-carboxylate (B85, 50.00 mg, 0.15 mmol), 2-bromo-4,6-dimethylpyrazolo[1,5-a]pyrazine (52.26 mg, 0.23 mmol), Pd(AcO)₂ (3.46 mg, 0.02 mmol), Pivalic acid (10.23 mg, 0.10 mmol), PCy3·HBF4 (11.35 mg, 0.03 mmol), K₂CO (127.79 mg, 0.92 mmol), and toluene (3.00 mL) were combined in a sealed tube under a nitrogen atmosphere. The reaction mixture was stirred for 16 h at 110° C., then extracted with ethyl acetate (3×10 mL). The organic layers were combined, washed with a saturated NaCl solution (1×10 mL), dried over anhydrous sodium sulfate, and concentrated in vacuo to give a residue. The residue was purified by silica gel column chromatography with ethyl acetate/petroleum ether (1:4) to afford tert-butyl 4-(2-[4,6-dimethylpyrazolo[1,5-a]pyrazin-2-yl]thieno[2,3-d][1,3]thiazol-5-yl)piperidine-1-carboxylate (50.00 mg, 69.09%) as a solid. LCMS (ES, m/z):470 [M+H]⁺.

### Synthesis of Compound 133

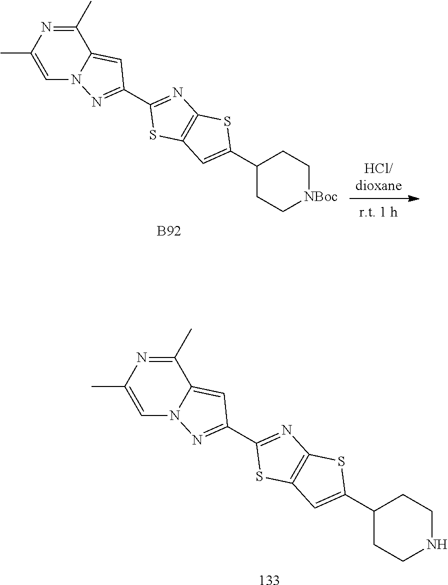 [SMILES: "B92" ; Cc1cn2nc(-c3nc4sc(C5CCN(C(=O)OC(C)(C)C)CC5)cc4s3)cc2c(C)n1 ; Cc1cn2nc(-c3nc4sc(C5CCNCC5)cc4s3)cc2c(C)n1 ; "133"]

Tert-butyl 4-(2-[4,6-dimethylpyrazolo[1,5-a]pyrazin-2-yl]thieno[2,3-d][1,3]thiazol-5-yl)piperidine-1-carboxylate (50.00 mg, 0.11 mmol) and HCl (gas) in 1,4-dioxane (5.00 mL, 87.59 mmol) were combined and stirred for 1 h at room temperature. The reaction mixture was concentrated in vacuo to give a residue. The residue was purified by Prep-HPLC (Condition 5, Gradient 2) to afford 4-(2-[4,6-dimethylpyrazolo[1,5-a]pyrazin-2-yl]thieno[2,3-d][1,3]thiazol-5-yl)piperidine (6.1 mg, 15.51%) as a solid. LCMS (ES, m/z):370 [M+H]⁺. ¹H NMR (400 MHz, DMSO-d₆) δ 8.56 (s, 1H), 7.52 (d, J=1.0 Hz, 1H), 7.31 (d, J=1.0 Hz, 1H), 3.08-2.96 (m, 3H), 2.73 (s, 3H), 2.61 (td, J=12.1, 2.4 Hz, 2H), 2.44 (d, J=1.0 Hz, 3H), 1.99-1.91 (m, 2H), 1.56 (qd, J=12.1, 3.8 Hz, 2H).

### Example 26: Synthesis of Compound 139

### Synthesis of Intermediate B93

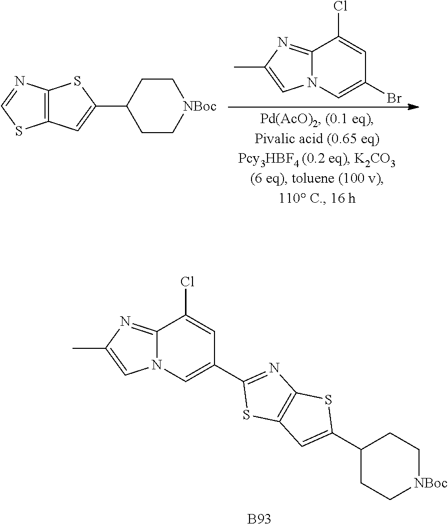 [SMILES: CC(C)(C)OC(=O)N1CCC(c2cc3scnc3s2)CC1 ; Cc1cn2cc(Br)cc(Cl)c2n1 ; "B93" ; Cc1cn2cc(-c3nc4sc(C5CCN(C(=O)OC(C)(C)C)CC5)cc4s3)cc(Cl)c2n1]

Tert-butyl 4-[thieno[2,3-d][1,3]thiazol-5-yl]piperidine-1-carboxylate (B85, 50.00 mg, 0.15 mmol), 6-bromo-8-chloro-2-methylimidazo[1,2-a]pyridine (56.75 mg, 0.23 mmol), Pd(AcO)₂ (3.46 mg, 0.02 mmol), Pivalic acid (10.23 mg, 0.10 mmol), PCy₃.HBF₄ (11.35 mg, 0.03 mmol), K₂CO₃ (127.79 mg, 0.93 mmol) and toluene (3.00 mL) were combined in a sealed tube under a nitrogen atmosphere. The reaction mixture was stirred for 16 h at 110° C., then extracted with ethyl acetate (3×10 mL). The organic layers combined, washed with saturated NaCl solution (1×10 ml), dried over anhydrous sodium sulfate, and concentrated in vacuo to give a residue. The residue was purified by silica gel column chromatography with ethyl acetate/petroleum ether (1:4) to afford tert-butyl 4-(2-[8-chloro-2-methylimidazo[1,2-a]pyridin-6-yl]thieno[2,3-d][1,3]thiazol-5-yl)piperidine-1-carboxylate (37.00 mg, 49.10%) as a solid. LCMS (ES, m/z):489 [M+H]⁺.

### Synthesis of Compound 139

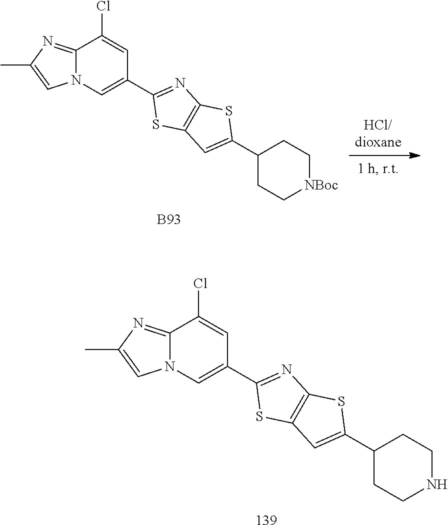 [SMILES: "B93" ; Cc1cn2cc(-c3nc4sc(C5CCN(C(=O)OC(C)(C)C)CC5)cc4s3)cc(Cl)c2n1 ; Cc1cn2cc(-c3nc4sc(C5CCNCC5)cc4s3)cc(Cl)c2n1 ; "139"]

Tert-butyl 4-(2-[8-chloro-2-methylimidazo[1,2-a]pyridin-6-yl]thieno[2,3-d][1,3]thiazol-5-yl)piperidine-1-carboxylate (37.00 mg, 0.076 mmol) and HCl(gas)in 1,4-dioxane (5.00 mL, 87.587 mmol, 1157.69 equiv) were combined and stirred for 1 h at room temperature. The reaction mixture was concentrated in vacuo to give a residue. The residue was purified by Prep-HPLC (Condition 5, Gradient 3) to afford 4-(2-[8-chloro-2-methylimidazo[1,2-a]pyridin-6-yl]thieno 2,3-d][1,3]thiazol-5-yl)piperidine (7.2 mg, 24.47%) as a solid. LCMS (ES, m/z):389 [M+H]⁺. ¹H NMR (400 MHz, DMSO-d₆) δ 9.27 (d, J=1.6 Hz, 1H), 7.90 (dd, J=14.4, 1.3 Hz, 2H), 7.31 (d, J=1.0 Hz, 1H), 3.00 (ddt, J=15.6, 11.5, 3.4 Hz, 3H), 2.60 (td, J=12.1, 2.4 Hz, 2H), 2.39 (d, J=0.9 Hz, 3H), 1.98-1.89 (m, 2H), 1.57 (dd, J=12.2, 3.9 Hz, 2H).

### Example 27: Synthesis of Compound 140

### Synthesis of Intermediate B94

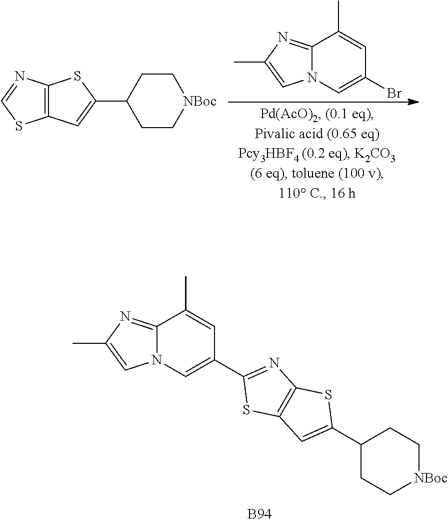 [SMILES: CC(C)(C)OC(=O)N1CCC(c2cc3scnc3s2)CC1 ; Cc1cn2cc(Br)cc(C)c2n1 ; "B94" ; Cc1cn2cc(-c3nc4sc(C5CCN(C(=O)OC(C)(C)C)CC5)cc4s3)cc(C)c2n1]

Tert-butyl 4-[thieno[2,3-d][1,3]thiazol-5-yl]piperidine-1-carboxylate (B85, 50.00 mg, 0.15 mmol), 6-bromo-2,8-dimethylimidazo[1,2-a]pyridine (52.03 mg, 0.23 mmol), Pd(AcO)₂ (3.46 mg, 0.02 mmol), Pivalic acid (10.23 mg, 0.100 mmol), PCy₃HBF₄ (11.35 mg, 0.031 mmol), K₂CO₃ (127.79 mg, 0.92 mmol), and toluene (3.00 mL) were combined in a sealed tube under a nitrogen atmosphere. The reaction mixture was stirred for 16 h at 110° C., then extracted with ethyl acetate (3×10 mL). The organic layers were combined, washed with a saturated NaCl solution (1×10 mL), dried over anhydrous sodium sulfate, and concentrated in vacuo to give a residue. The residue was purified by silica gel column chromatography with ethyl acetate/petroleum ether (1:4) to afford tert-butyl 4-(2-[2,8-dimethylimidazo[1,2-a]pyridin-6-yl]thieno [2,3-d][1,3]thiazol-5-yl)piperidine-1-carboxylate (40.00 mg, 55.39%) as a solid. LCMS (ES, m/z):469 [M+H]⁺.

### Synthesis of Compound 140

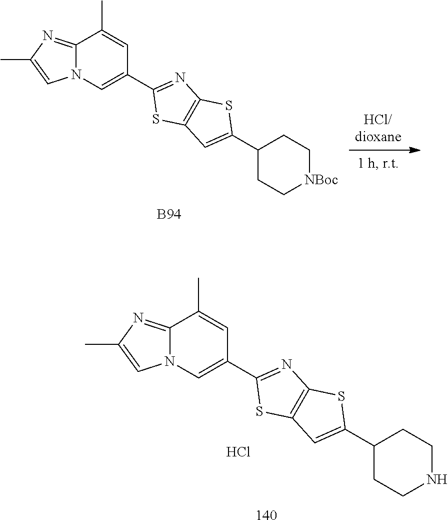 [SMILES: "B94" ; Cc1cn2cc(-c3nc4sc(C5CCN(C(=O)OC(C)(C)C)CC5)cc4s3)cc(C)c2n1 ; Cc1cn2cc(-c3nc4sc(C5CCNCC5)cc4s3)cc(C)c2n1 ; Cl ; "140"]

Into a 25-mL round-bottom flask, was placed tert-butyl 4-(2-[2,8-dimethylimidazo[1,2-a]pyridin-6-yl]thieno[2,3-d][1,3]thiazol-5-yl)piperidine-1-carboxylate (40.00 mg, 0.09 mmol, 1.00 equiv), HCl(gas)in 1,4-dioxane (5.00 mL, 87.59 mmol, 1026.15 equiv). The resulting solution was stirred for 1 hr at room temperature. The resulting mixture was concentrated under vacuum. The crude product was purified by Prep-HPLC (Condition 7, Gradient 1) to afford 4-(2-[2,8-dimethylimidazo[1,2-a]pyridin-6-yl]thieno[2,3-d][1,3]thiazol-5-yl)piperidine hydrochloride (10.10 mg, 32.11%) as a solid. LCMS (ES, m/z):369 [M+H]⁺. 1H NMR (400 MHz, DMSO-d₆) δ 14.77 (s, 1H), 9.46 (s, 1H), 8.92 (s, 1H), 8.79 (d, J=10.9 Hz, 1H), 8.21 (s, 1H), 8.11 (s, 1H), 7.43 (d, J=1.0 Hz, 1H), 3.32 (m, 2H), 3.25 (m, 1H), 3.01 (d, J=11.9 Hz, 2H), 2.66 (s, 3H), 2.47 (s, 3H), 2.19 (d, J=13.3 Hz, 2H), 1.98-1.83 (m, 2H).

### Example 28: Synthesis of Compound 127

### Synthesis of Intermediate B95

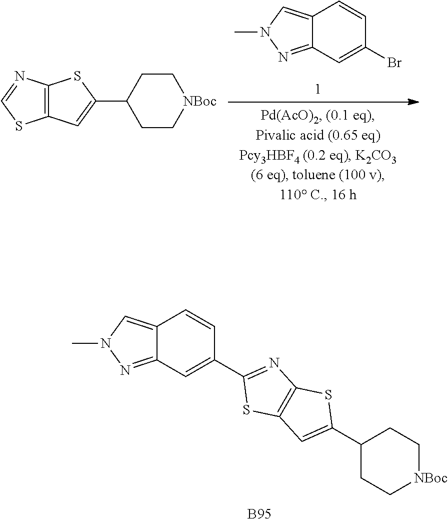 [SMILES: Cn1cc2ccc(Br)cc2n1 ; CC(C)(C)OC(=O)N1CCC(c2cc3scnc3s2)CC1 ; "B95" ; Cn1cc2ccc(-c3nc4sc(C5CCN(C(=O)OC(C)(C)C)CC5)cc4s3)cc2n1]

Tert-butyl 4-[thieno[2,3-d][1,3]thiazol-5-yl]piperidine-1-carboxylate (B85, 50.00 mg, 0.15 mmol), 6-bromo-2-methylindazole (48.79 mg, 0.23 mmol), Pd(AcO)₂ (3.46 mg, 0.02 mmol), Pivalic acid (10.23 mg, 0.10 mmol), PCy₃.HBF₄ (11.35 mg, 0.03 mmol), K₂CO₃ (127.79 mg, 0.93 mmol), and toluene (3.00 ml) were combined in a sealed tube under a nitrogen atmosphere. The reaction mixture was stirred for 16 h at 110° C., then extracted with ethyl acetate (3×10 mL). The organic layers were combined, washed with a saturated NaCl solution (1×10 mL), dried over anhydrous sodium sulfate, and concentrated in vacuo to give a residue. The residue was purified by silica gel column chromatography with ethyl acetate/petroleum ether (1:4) to afford tert-butyl 4-[2-(2-methylindazol-6-yl)thieno[2,3-d][1,3]thiazol-5-yl]piperidine-1-carboxylate (37.00 mg, 52.81%) as a solid. LCMS (ES, m/z):455 [M+H]⁺.

### Synthesis of Compound 127

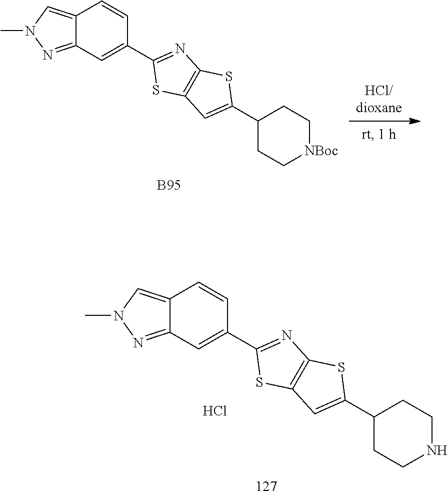 [SMILES: "B95" ; Cn1cc2ccc(-c3nc4sc(C5CCN(C(=O)OC(C)(C)C)CC5)cc4s3)cc2n1 ; Cn1cc2ccc(-c3nc4sc(C5CCNCC5)cc4s3)cc2n1 ; Cl ; "127"]

Tert-butyl 4-[2-(2-methylindazol-6-yl)thieno [2,3-d][1,3]thiazol-5-yl]piperidine-1-carboxylate (37.00 mg, 0.08 mmol) and HCl(gas)in 1,4-dioxane (5.00 mL) were combined and stirred for 1 h at room temperature. The reaction mixture was concentrated in vacuo to give a residue. The residue was purified by Prep-HPLC (Condition 6, Gradient 1) to afford 2-methyl-6-[5-(piperidin-4-yl)thieno [2,3-d][1,3]thiazol-2-yl]indazole hydrochloride (6.60 mg, 22.88%) as a solid. LCMS (ES, m/z):355 [M+H]⁺. ¹H NMR (400 MHz, DMSO-d₆) δ 8.80 (s, 1H), 8.55 (s, 1H), 8.44 (s, 1H), 8.21 (q, J=1.1 Hz, 1H), 7.86 (dd, J=8.8, 0.9 Hz, 1H), 7.67 (dd, J=8.7, 1.5 Hz, 1H), 7.37 (d, J=1.0 Hz, 1H), 4.22 (s, 3H), 3.39 (d, J=12.6 Hz, 2H), 3.08 (d, J=12.0 Hz, 1H), 3.02 (d, J=11.9 Hz, 2H), 2.23-2.15 (m, 2H), 1.95-1.80 (m, 2H).

### Example 29: Synthesis of Compound 149

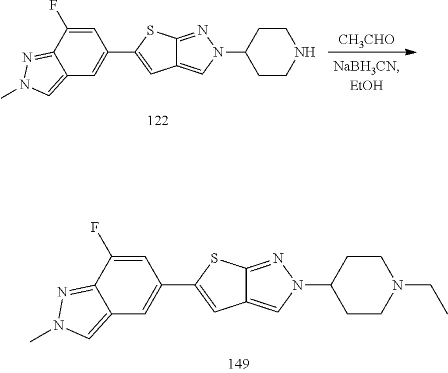

A solution of 7-fluoro-2-methyl-5-[2-(piperidin-4-yl)thieno[2,3-c]pyrazol-5-yl]indazole (100 mg, 0.281 mmol, 1.00 equiv) and acetaldehyde (24.79 mg, 0.562 mmol, 2 equiv) in MeOH (1 mL) was stirred for 1 h at room temperature under nitrogen atmosphere. NaBH₃CN (35.36 mg, 0.562 mmol, 2 equiv) was then added, and the resulting mixture was stirred for 2 h at room temperature under nitrogen atmosphere. The resulting mixture was diluted with water (5 mL), extracted with EtOAc (2×30 mL). The combined organic layers were washed with brine (2×30 mL), dried over anhydrous Na₂SO₄, and the crude product was purified by Prep-HPLC (Condition 2, Gradient 4) to afford 5-[2-(1-ethylpiperidin-4-yl)thieno[2,3-c]pyrazol-5-yl]-7-fluoro-2-methylindazole (28.2 mg, 26.14%) as a solid. LCMS (ES, m/z): 384 [M+H]⁺. ¹H NMR (400 MHz, DMSO-d6) δ 8.49 (d, J=2.8 Hz, 1H), 8.14 (s, 1H), 7.71 (d, J=1.4 Hz, 1H), 7.53 (dd, J=13.1, 1.4 Hz, 1H), 7.48 (s, 1H), 4.29 (s, 1H), 4.21 (s, 3H), 2.99 (t, J=4.7 Hz, 2H), 2.38 (q, J=7.2 Hz, 2H), 2.05-2.00 (m, 6H), 1.03 (t, J=7.2 Hz, 3H).

### Example 30: Synthesis of Compound 150

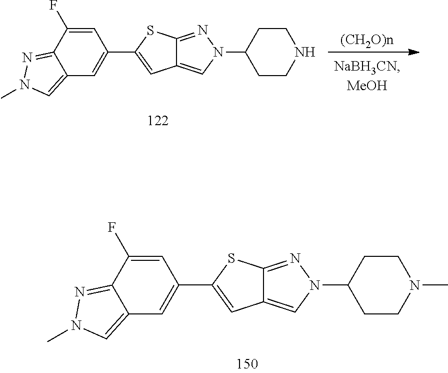

A solution of 7-fluoro-2-methyl-5-[2-(piperidin-4-yl)thieno[2,3-c]pyrazol-5-yl]indazole (100 mg, 0.281 mmol, 1.00 equiv) and (HCHO)n (0.5 mL, Infinity mmol, Infinity equiv) in MeOH (1 mL) was stirred for 1 h at room temperature under nitrogen atmosphere. Were added NaBH₃CN (35.36 mg, 0.562 mmol, 2 equiv) at room temperature. The resulting mixture was stirred for 2 h at room temperature under nitrogen atmosphere. The resulting mixture was diluted with water (5 mL). The resulting mixture was extracted with EtOAc (2×5 mL). The combined organic layers were washed with brine (2×5 mL), dried over anhydrous Na₂SO₄. After filtration, the filtrate was concentrated under reduced pressure. The crude product was purified by Prep-HPLC (Condition 2, Gradient 4) to afford 7-fluoro-2-methyl-5-[2-(1-methylpiperidin-4-yl)thieno[2,3-c]pyrazol-5-yl]indazole (47.1 mg, 45.31%) as a solid. LCMS (ES, m/z): 370 [M+H]^{+ 1}H NMR (400 MHz, DMSO-d6) δ 8.49 (d, J=2.8 Hz, 1H), 8.13 (s, 1H), 7.71 (d, J=1.4 Hz, 1H), 7.53 (dd, J=13.1, 1.4 Hz, 1H), 7.48 (s, 1H), 4.29 (s, 1H), 4.21 (s, 3H), 2.90 (d, J=7.3 Hz, 2H), 2.24 (s, 3H), 2.16-2.01 (m, 6H).

### Example 31: Synthesis of Compound 152

### Synthesis of Intermediate B96

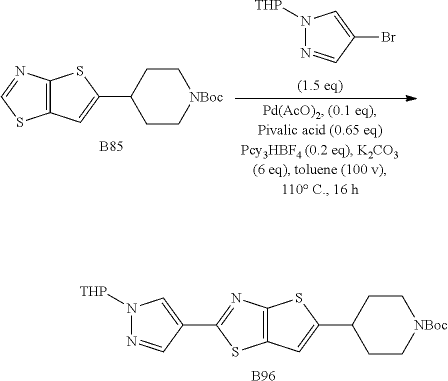

Into a 8-mL sealed tube purged and maintained with an inert atmosphere of nitrogen was placed tert-butyl 4-[thieno[2,3-d][1,3]thiazol-5-yl]piperidine-1-carboxylate (B85, 50.00 mg, 0.15 mmol, 1.00 equiv), 4-bromo-1-(oxan-2-yl)pyrazole (53.42 mg, 0.23 mmol, 1.50 equiv), Pivalic acid (10.23 mg, 0.10 mmol, 0.65 equiv), PCy₃.HBF₄ (11.35 mg, 0.03 mmol, 0.20 equiv), Pd(AcO)₂ (3.46 mg, 0.02 mmol, 0.10 equiv), K₂CO₃ (127.79 mg, 0.93 mmol, 6.00 equiv), and toluene (3.00 mL). The resulting solution was stirred for 10 hr at 100° C., then extracted with 3×10 mL of ethyl acetate. The organic layers were combined and washed with 1×10 ml of sat. NaCl, then dried over anhydrous sodium sulfate and concentrated under vacuum. The residue was applied onto a silica gel column with ethyl acetate/petroleum ether (1:5). This resulted in 30.00 mg (41.02%) of tert-butyl 4-[2-[1-(oxan-2-yl)pyrazol-4-yl]thieno[2,3-d][1,3]thiazol-5-yl]piperidine-1-carboxylate as a solid. LCMS (ES, m/z):475 [M+H]⁺.

### Synthesis of Compound 152

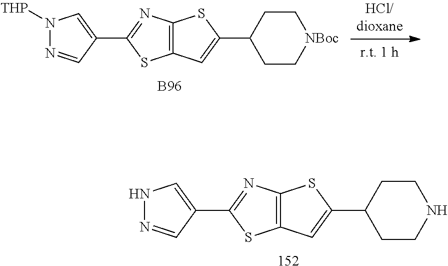

Into a 25-mL round-bottom flask was placed tert-butyl 4-[2-[1-(oxan-2-yl)pyrazol-4-yl]thieno[2,3-d][1,3]thiazol-5-yl]piperidine-1-carboxylate (B96, 30.00 mg, 0.06 mmol, 1.00 equiv), HCl(gas)in 1,4-dioxane (3.00 ug, 0.000 mmol). The resulting solution was stirred for 1 hr at room temperature then concentrated under vacuum. The crude product was purified by Prep-HPLC (Condition 5, Gradient 6) to afford 4-[2-(1H-pyrazol-4-yl)thieno[2,3-d][1,3]thiazol-5-yl]piperidine (2.3 mg, 12.53%) as a solid. LCMS (ES, m/z):291 [M+H]⁺. ¹H NMR (400 MHz, DMSO-d₆) δ 13.34 (s, 1H), 8.21 (s, 2H), 7.22 (d, J=1.0 Hz, 1H), 3.12-3.05 (m, 2H), 3.05-2.96 (m, 1H), 2.67 (td, J=12.2, 2.5 Hz, 2H), 1.97 (d, J=12.5 Hz, 2H), 1.55 (dd, J=12.3, 3.9 Hz, 2H).

### Example 32: Synthesis of Compound 151

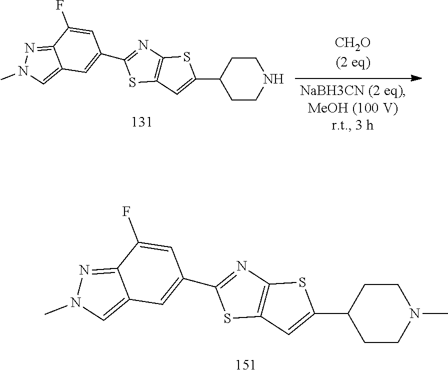

Into a 8-mL sealed tube was placed HCHO (5.64 mg, 0.20 mmol, 2.00 equiv), NaBH₃CN (11.81 mg, 0.188 mmol, 2.00 equiv), MeOH (2.00 mL), and 7-fluoro-2-methyl-5-[5-(piperidin-4-yl)thieno[2,3-d][1,3]thiazol-2-yl]indazole (131, 35.00 mg, 0.09 mmol, 1.00 equiv), and the resulting solution was stirred for 3 hr at room temperature. The solution was then extracted with 3×10 mL of ethyl acetate and the organic layers combined, washed with 1×10 ml of sat. NaCl, dried over anhydrous sodium sulfate, and concentrated under vacuum. The crude product was purified by Prep-HPLC (Condition 2, Gradient 1) to afford 7-fluoro-2-methyl-5-[5-(1-methylpiperidin-4-yl)thieno[2,3-d][1,3]thiazol-2-yl]indazole (6.00 mg, 16.52%) as a solid. LCMS (ES, m/z):387 [M+H]⁺. 1H NMR (400 MHz, DMSO-d₆) δ 8.62 (d, J=2.7 Hz, 1H), 8.23 (d, J=1.3 Hz, 1H), 7.64 (dd, J=12.6, 1.4 Hz, 1H), 7.33 (d, J=1.0 Hz, 1H), 4.24 (s, 3H), 3.09 (s, 3H), 2.45-2.40 (m, 5H), 2.09 (d, J=12.7 Hz, 2H), 1.79-1.72 (m, 2H).

### Example 33: Synthesis of Compound 153

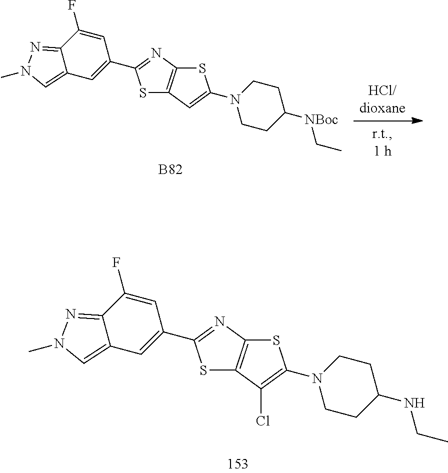

Into a 25-mL round-bottom flask was placed tert-butyl N-ethyl-N-[1-[2-(7-fluoro-2-methylindazol-5-yl)thieno[2,3-d][1,3]thiazol-5-yl]piperidin-4-yl]carbamate (B82, 50.00 mg, 0.10 mmol, 1.00 equiv), HCl(gas)in 1,4-dioxane (5.00 mL, 87.59 mmol, 903.32 equiv). The resulting solution was stirred for 1 hr at room temperature, then concentrated under vacuum. The crude product was purified by Prep-HPLC (Condition 5, Gradient 4) to afford 1-[6-chloro-2-(7-fluoro-2-methylindazol-5-yl)thieno[2,3-d][1,3]thiazol-5-yl]-N-ethylpiperidin-4-amine (1.90 mg, 4.35%) as a solid. LCMS (ES, m/z):450 [M+H]⁺. ¹H NMR (400 MHz, DMSO-d₆) δ 8.63 (d, J=2.7 Hz, 1H), 8.24 (s, 1H), 7.64 (d, J=12.4 Hz, 1H), 4.24 (s, 3H), 3.60-3.40 (m, 2H), 2.90-2.80 (m, 3H), 2.72 (d, J=7.2 Hz, 2H), 1.99 (d, J=12.4 Hz, 2H), 1.55 (q, J=10.6 Hz, 2H), 1.09 (t, J=7.1 Hz, 3H).

### Example 34: Synthesis of Compound 154

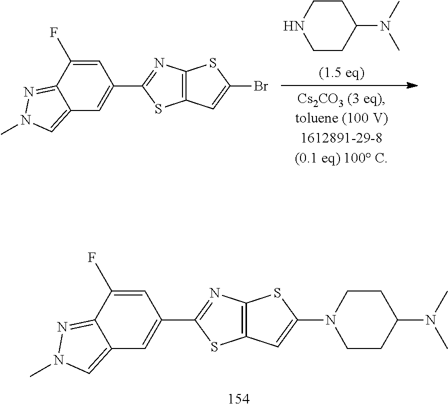

Into a 8-mL sealed tube purged and maintained with an inert atmosphere of nitrogen was placed 5-[5-bromothieno[2,3-d][1,3]thiazol-2-yl]-7-fluoro-2-methylindazole (100.00 mg, 0.27 mmol, 1.00 equiv), N,N-dimethylpiperidin-4-amine (52.23 mg, 0.41 mmol, 1.50 equiv), Pd-PEPPSI-IPentCl 2-methylpyridine (o-picoline (22.84 mg, 0.03 mmol, 0.10 equiv), Cs₂CO₃ (265.44 mg, 0.82 mmol, 3.00 equiv) and toluene (3.00 mL), and the resulting solution was stirred for 10 hr at 100° C. The solution was then extracted with 3×10 mL of ethyl acetate and the organic layers combined, washed with 1×10 ml of sat. NaCl, dried over anhydrous sodium sulfate, then concentrated under vacuum. The residue was applied onto a silica gel column with ethyl acetate/petroleum ether (1:4). The crude product was purified by Prep-HPLC (Condition 5, Gradient 5) to afford 1-[2-(7-fluoro-2-methylindazol-5-yl)thieno[2,3-d][1,3]thiazol-5-yl]-N,N-dimethylpiperidin-4-amine (18.20 mg, 16.13%) as a solid. LCMS (ES, m/z):416 [M+H]⁺. ¹H NMR (400 MHz, DMSO-d₆) δ 8.56 (d, J=2.8 Hz, 1H), 8.08 (s, 1H), 7.59 (d, J=12.6 Hz, 1H), 6.49 (s, 1H), 4.22 (s, 3H), 3.58 (d, J=12.0 Hz, 2H), 2.90 (td, J=12.2, 2.8 Hz, 2H), 2.27 (s, 1H), 2.20 (s, 6H), 1.86 (d, J=11.6 Hz, 2H), 1.55 (qd, J=11.8, 4.0 Hz, 2H).

### Example 35: Synthesis of Compound 250

### Synthesis of Intermediate B97

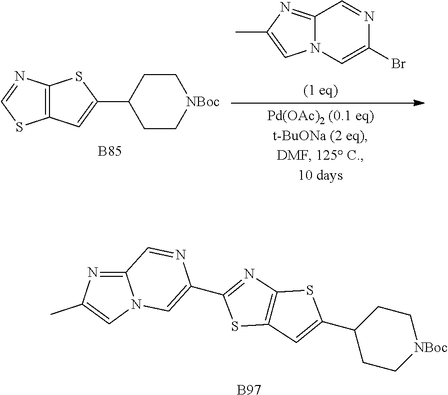

To a stirred mixture of tert-butyl 4-{thieno[2,3-d][1,3]thiazol-5-yl}piperidine-1-carboxylate (80.00 mg, 0.25 mmol, 1.00 equiv) and 6-bromo-2-methylimidazo[1,2-a]pyrazine (52.28 mg, 0.25 mmol, 1.00 equiv) in DMF (5 mL) was added Pd(OAc)₂ (5.54 mg, 0.03 mmol, 0.10 equiv) and t-BuONa (47.39 mg, 0.49 mmol, 2.00 equiv). The reaction mixture was stirred for 10 days at 125° C. under nitrogen atmosphere. The resulting mixture was extracted with ethyl acetate (3×10 mL). The combined organic layers were washed with saturated NaCl (1×10 mL), dried over anhydrous Na₂SO₄, and filtered. After filtration, the filtrate was concentrated under reduced pressure to give a residue. The residue was purified by silica gel column chromatography, eluted with PE/EA (1:1) to afford tert-butyl 4-(2-{2-methylimidazo[1,2-a]pyrazin-6-yl}thieno[2,3-d][1,3]thiazol-5-yl)piperidine-1-carboxylate (30.00 mg, 26.71%) as a solid. LCMS (ES, m/z):456 [M+H]⁺.

### Synthesis of Compound 205

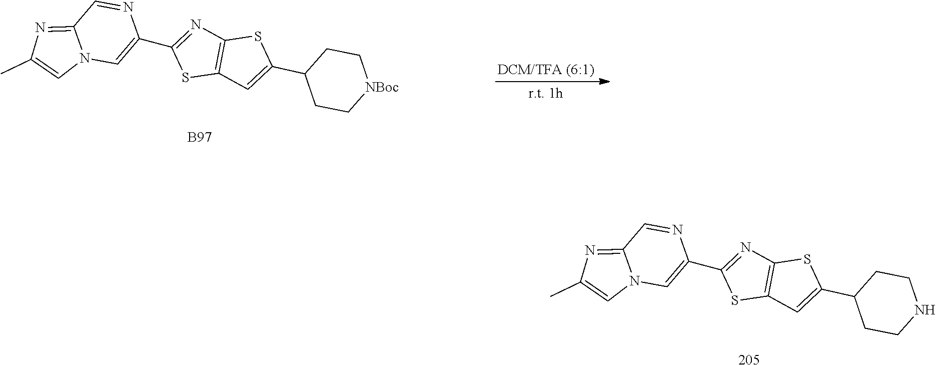 [SMILES: "B97" ; Cc1cn2cc(-c3nc4sc(C5CCN(C(=O)OC(C)(C)C)CC5)cc4s3)ncc2n1 ; Cc1cn2cc(-c3nc4sc(C5CCNCC5)cc4s3)ncc2n1 ; "205"]

A mixture of tert-butyl 4-(2-{2-methylimidazo[1,2-a]pyrazin-6-yl}thieno[2,3-d][1,3]thiazol-5-yl) piperidine-1-carboxylate (30.00 mg, 0.07 mmol, 1.00 equiv) and DCM/TFA (3 mL, 6:1) was stirred for 1 h at room temperature. The resulting mixture was concentrated under reduced pressure to give a residue. The residue was purified by reverse flash chromatography (Condition 2, Gradient 4) to afford 4-(2-{2-methylimidazo[1,2-a]pyrazin-6-yl}thieno[2,3-d][1,3]thiazol-5-yl)piperidine (2.20 mg, 9.40%) as a solid. LCMS (ES, m/z):356 [M+H]⁺. ¹H NMR (400 MHz, DMSO-d₆) δ 9.38 (d, J=1.5 Hz, 1H), 9.02 (d, J=1.4 Hz, 1H), 8.03 (s, 1H), 7.31 (d, J=1.0 Hz, 1H), 3.04 (d, J=3.2 Hz, 3H), 2.61 (td, J=12.1, 2.4 Hz, 2H), 2.45 (s, 3H), 1.99-1.90 (m, 2H), 1.55 (dd, J=12.1, 3.8 Hz, 2H).

### Example 36: Synthesis of Compound 199

### Synthesis of Intermediate B98

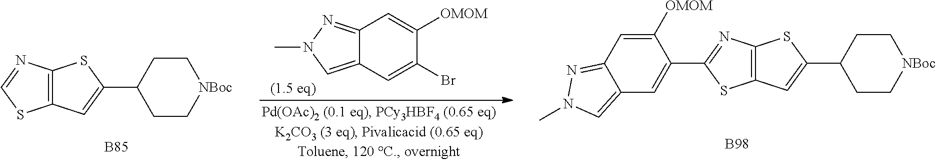

To a stirred mixture of tert-butyl 4-{thieno[2,3-d][1,3]thiazol-5-yl}piperidine-1-carboxylate (60.00 mg, 0.19 mmol, 1.00 equiv) and 5-bromo-6-(methoxymethoxy)-2-methylindazole (75.20 mg, 0.28 mmol, 1.50 equiv) in toluene (3.00 mL) was added Pd(OAc)₂ (4.15 mg, 0.02 mmol, 0.10 equiv), PCy₃HBF₄ (44.26 mg, 0.12 mmol, 0.65 equiv), Pivalic acid (12.28 mg, 0.12 mmol, 0.65 equiv), and K₂CO₃ (76.67 mg, 0.56 mmol, 3.00 equiv). The reaction mixture was stirred for 4 days at 120° C. under nitrogen atmosphere, then extracted with ethyl acetate (3×10 mL). The combined organic layers were washed with saturated NaCl (1×10 mL), dried over anhydrous Na₂SO₄, and filtered. After filtration, the filtrate was concentrated under reduced pressure to give a residue. The residue was purified by silica gel column chromatography, eluted with CH₂Cl₂/MeOH (10:1) to afford tert-butyl 4-{2-[6-(methoxymethoxy)-2-methylindazol-5-yl]thieno[2,3-d][1,3]thiazol-5-yl}piperidine-1-carboxylate (30.00 mg, 31.52%) as a solid. LCMS (ES, m/z): 515 [M+H]⁺.

### Synthesis of Compound 199

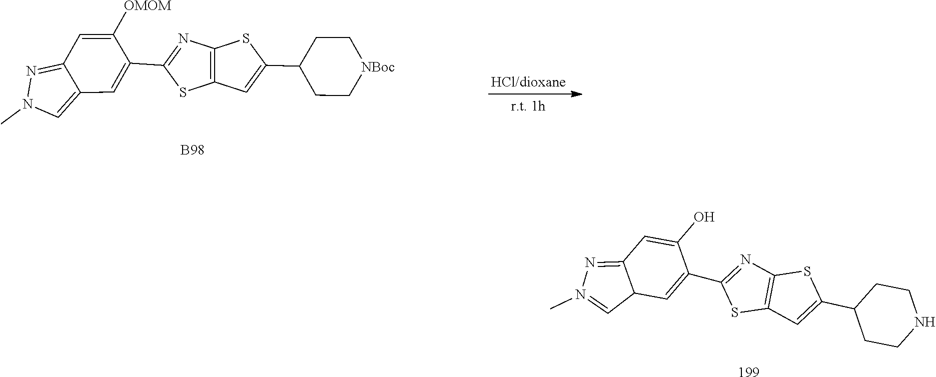

A mixture of tert-butyl 4-{2-[6-(methoxymethoxy)-2-methylindazol-5-yl]thieno[2,3-d][1,3]thiazol-5-yl} piperidine-1-carboxylate (30.00 mg, 0.06 mmol, 1.00 equiv) and HCl (gas) in 1,4-dioxane (5 mL) was stirred for 1 h at room temperature. The resulting mixture was concentrated under reduced pressure to give a residue. The residue was purified by reverse flash chromatography (Condition 8, Gradient 1) to afford 2-methyl-5-[5-(piperidin-4-yl)thieno[2,3-d][1,3]thiazol-2-yl]indazol-6-ol hydrochloride (2.40 mg, 10.12%) as a solid. LCMS (ES, m/z):371 [M+H]⁺. ¹H NMR (400 MHz, DMSO-d₆) δ 10.97 (s, 1H), 8.91 (d, J 11.4 Hz, 1H), 8.66 (d, J=12.3 Hz, 1H), 8.62 (s, 1H), 8.39 (s, 1H), 7.30 (d, J=1.0 Hz, 1H), 7.00 (s, 1H), 4.12 (s, 3H), 3.38 (d, J=12.6 Hz, 2H), 3.33-3.23 (m, 1H), 3.07 (d, J=12.0 Hz, 2H), 2.19 (d, J=13.6 Hz, 2H), 1.88 (qd, J=13.0, 3.9 Hz, 2H).

### Example 37: Synthesis of Compound 206

### Synthesis of Intermediate B99

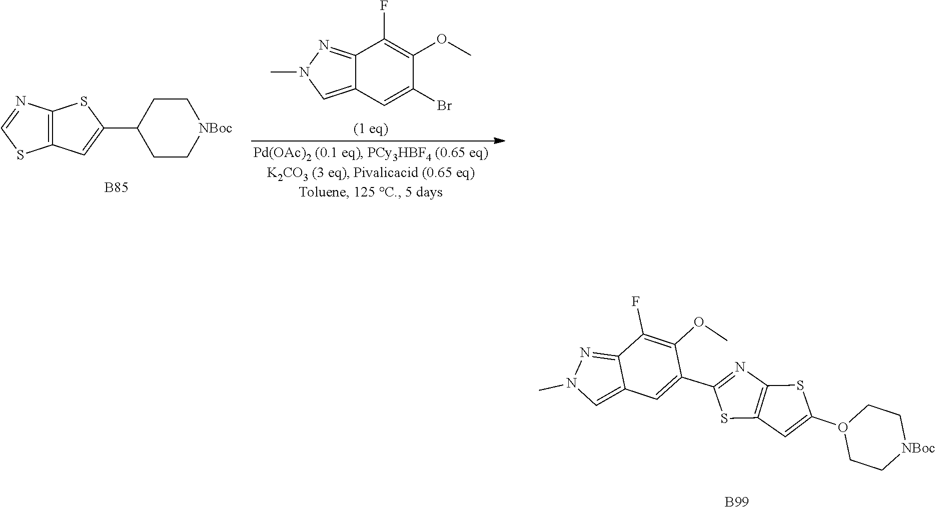

To a stirred mixture of tert-butyl 4-{thieno[2,3-d][1,3]thiazol-5-yl}piperidine-1-carboxylate (100.00 mg, 0.31 mmol, 1.00 equiv), 5-bromo-7-fluoro-6-methoxy-2-methylindazole (79.85 mg, 0.31 mmol, 1.00 equiv), Pd(OAc)₂ (6.92 mg, 0.03 mmol, 0.10 equiv), and PCy₃HBF₄ (56.18 mg, 0.20 mmol, 0.65 equiv) in toluene (5 mL) was added K₂CO₃ (127.79 mg, 0.92 mmol, 3.0 equiv) and Pivalic acid (20.46 mg, 0.200 mmol, 0.65 equiv). The reaction mixture was stirred for 5 days at 125° C. under nitrogen atmosphere, then extracted with ethyl acetate (3×10 mL). The combined organic layers were washed with saturated NaCl (1×10 mL), dried over anhydrous Na₂SO₄, and filtered. After filtration, the filtrate was concentrated under reduced pressure to give a residue. The residue was purified by silica gel column chromatography, eluted with PE/EA (5:1) to afford tert-butyl 4-[2-(7-fluoro-6-methoxy-2-methylindazol-5-yl)thieno [2,3-d][1,3] thiazol-5-yl]piperidine-1-carboxylate (65.00 mg, 41.96%) as a brown solid. LCMS (ES, m/z):503 [M+H]⁺.

### Synthesis of Compound 206

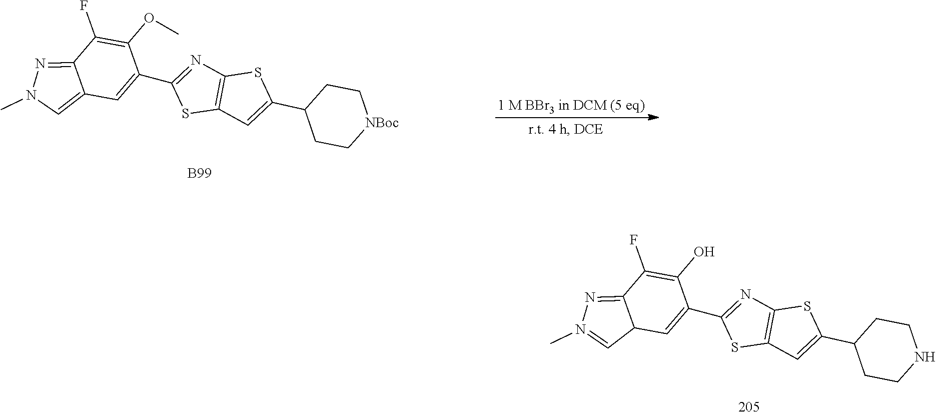

A mixture of tert-butyl 4-[2-(7-fluoro-6-methoxy-2-methylindazol-5-yl)thieno[2,3-d][1,3]thiazol-5-yl]piperidine-1-carboxylate (65.00 mg, 0.13 mmol, 1.00 equiv) and BBr₃ in DCM (1 M, 1.5 equiv) in DCE (5 mL) was stirred for 8 h at 60° C. The reaction mixture was quenched with water at room temperature, then concentrated under reduced pressure to give a residue. The residue was purified by reverse flash chromatography (Condition 2, Gradient 4) to afford 7-fluoro-2-methyl-5-[5-(piperidin-4-yl)thieno[2,3-d][1,3] thiazol-2-yl]indazol-6-ol (5.30 mg, 10.55%) as a solid. LCMS (ES, m/z):389 [M+H]⁺. ¹H NMR (400 MHz, DMSO-d₆) δ 8.43 (d, J=2.6 Hz, 1H), 8.31 (d, J=1.1 Hz, 1H), 7.24 (d, J=1.1 Hz, 1H), 4.16 (s, 3H), 3.15 (t, J=12.0 Hz, 3H) 2.71 (t, J=12.0 Hz, 2H), 2.06 (m, 2H), 1.65 (qd, J=12.1, 4.0 Hz, 2H).

### Example 38: Synthesis of Compound 200

### Synthesis of Intermediate B100

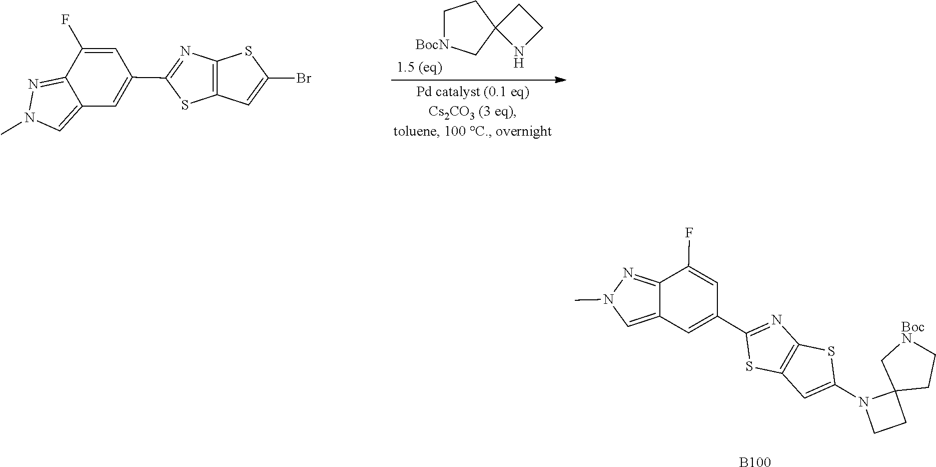 [SMILES: Cn1cc2cc(-c3nc4sc(Br)cc4s3)cc(F)c2n1 ; CC(C)(C)OC(=O)N1CCC2(CCN2)C1 ; "B100" ; Cn1cc2cc(-c3nc4sc(N5CCC56CCN(C(=O)OC(C)(C)C)C6)cc4s3)cc(F)c2n1]

To a mixture of 5-{5-bromothieno[2,3-d][1,3]thiazol-2-yl}-7-fluoro-2-methylindazole (60.00 mg, 0.16 mmol, 1.00 equiv) and tert-butyl 1,6-diazaspiro[3.4]octane-6-carboxylate (51.89 mg, 0.24 mmol, 1.50 equiv) in a mixture of dioxane/water (3 mL) was added Pd-PEPPSI-IPentCl 2-methylpyridine (o-picoline (13.71 mg, 0.02 mmol, 0.10 equiv) and Cs₂CO₃ (37.22 mg, 0.49 mmol, 3.00 equiv). The reaction mixture was stirred for 8 h at 100° C. under nitrogen atmosphere, then extracted with ethyl acetate (3×10 mL). The combined organic layers were washed with saturated NaCl (1×10 mL), dried over anhydrous Na₂SO₄, and filtered. After filtration, the filtrate was concentrated under reduced pressure to give a residue. The residue was purified by silica gel column chromatography, eluted with CH₂Cl₂/MeOH (10:1) to afford tert-butyl 1-[2-(7-fluoro-2-methylindazol-5-yl)thieno[2,3-d][1,3]thiazol-5-yl]-1,6-diazaspiro[3.4]octane-6-carboxylate (60 mg, 73.70%) as a solid. LCMS (ES, m/z): 500 [M+H]⁺.

### Synthesis of Compound 200

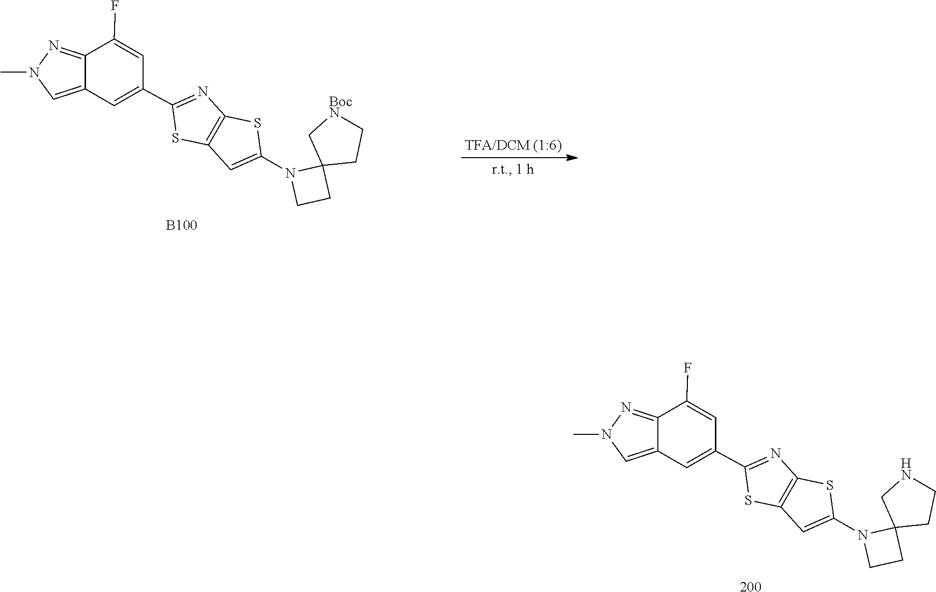 [SMILES: "B100" ; Cn1cc2cc(-c3nc4sc(N5CCC56CCN(C(=O)OC(C)(C)C)C6)cc4s3)cc(F)c2n1 ; Cn1cc2cc(-c3nc4sc(N5CCC56CCNC6)cc4s3)cc(F)c2n1 ; "200"]

A mixture of tert-butyl 1-[2-(7-fluoro-2-methylindazol-5-yl)thieno[2,3-d][1,3]thiazol-5-yl]-1,6-diazaspiro[3.4]octane-6-carboxylate (60.00 mg, 0.12 mmol, 1.00 equiv) in TFA/DCM (0.5 mL/3 mL) was stirred for 1 h at room temperature. The resulting mixture was concentrated under reduced pressure to give a residue. The residue was purified by reverse flash chromatography (Condition 5, Gradient 7) to afford 5-(5-{1,6-diazaspiro[3.4]octan-1-yl}thieno[2,3-d][1,3]thiazol-2-yl)-7-fluoro-2-methy-lindazole (5.60 mg, 11.67%) as a solid. LCMS (ES, m/z):400 [M+H]⁺. ¹H NMR (400 MHz, DMSO-d₆) δ 8.50 (d, J=2.8 Hz, 1H), 8.03 (d, J=1.3 Hz, 1H), 7.53 (dd, J=12.7, 1.4 Hz, 1H), 6.30 (s, 1H), 4.21 (s, 3H), 3.73 (dd, J=7.9, 6.5 Hz, 2H), 3.22 (d, J=11.5 Hz, 1H), 3.02-2.93 (m, 1H), 2.93 (m, 1H), 2.78 (m, 1H), 2.45 (m, 1H), 2.31 (m, 1H), 2.20 (m, 1H), 1.86 (ddd, J=13.0, 7.6, 4.9 Hz, 1H).

### Example 39: Synthesis of Compound 202

### Synthesis of Intermediate B101

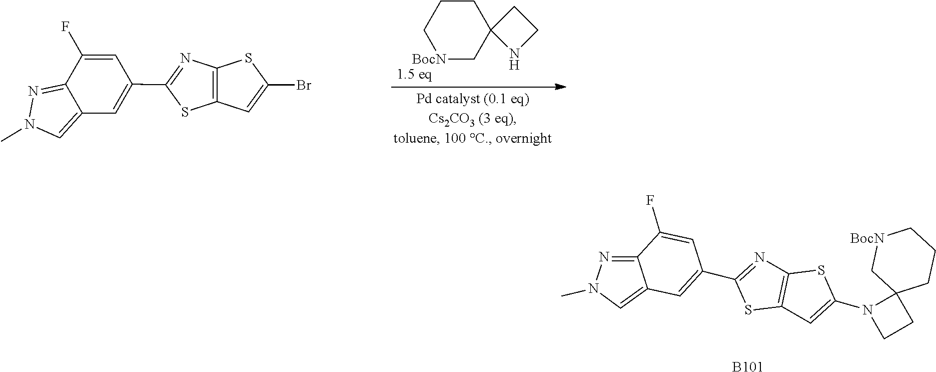 [SMILES: Cn1cc2cc(-c3nc4sc(Br)cc4s3)cc(F)c2n1 ; CC(C)(C)OC(=O)N1CCCC2(CCN2)C1 ; "B101" ; Cn1cc2cc(-c3nc4sc(N5CCC56CCCN(C(=O)OC(C)(C)C)C6)cc4s3)cc(F)c2n1]

To a mixture of 5-{5-bromothieno[2,3-d][1,3]thiazol-2-yl}-7-fluoro-2-methylindazole (150.00 mg, 0.41 mmol, 1.00 equiv) and tert-butyl 1,6-diazaspiro[3.5]nonane-6-carboxylate (138.28 mg, 0.61 mmol, 1.50 equiv) in toluene (10 mL) was added Pd-PEPPSI-IPentCl 2-methylpyridine (o-picoline (34.26 mg, 0.04 mmol, 0.10 equiv) and Cs₂CO₃ (398.16 mg, 1.22 mmol, 3.00 equiv) in portions at 100° C. under nitrogen atmosphere. The resulting mixture was stirred overnight, then extracted with ethyl acetate (3×10 mL). The combined organic layers were washed with saturated NaCl (1×10 mL), dried over anhydrous Na₂SO₄, and filtered. After filtration, the filtrate was concentrated under reduced pressure to give a residue. The residue was purified by silica gel column chromatography, eluted with PE/EA (1:1) to afford tert-butyl 1-[2-(7-fluoro-2-methylindazol-5-yl)thieno [2,3-d][1,3]thiazol-5-yl]-1,6-diazaspiro[3.5]nonane-6-carboxylate (131.00 mg, 62.61%) as an oil. LCMS (ES, m/z): 514 [M+H]⁺.

### Synthesis of Compound 202

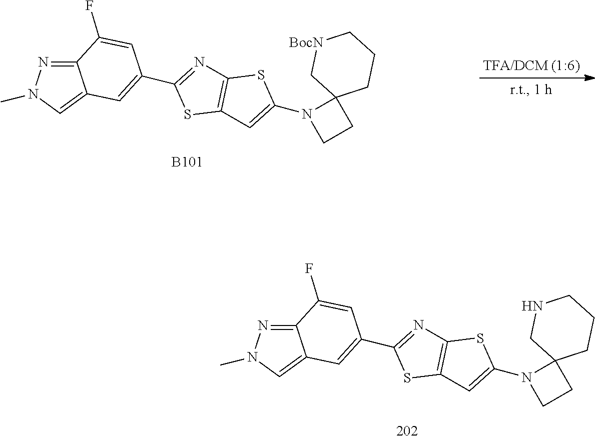 [SMILES: "B101" ; Cn1cc2cc(-c3nc4sc(N5CCC56CCCN(C(=O)OC(C)(C)C)C6)cc4s3)cc(F)c2n1 ; Cn1cc2cc(-c3nc4sc(N5CCC56CCCNC6)cc4s3)cc(F)c2n1 ; "202"]

A mixture of tert-butyl 1-[2-(7-fluoro-2-methylindazol-5-yl)thieno[2,3-d][1,3]thiazol-5-yl]-1,6-diazaspiro[3.5]nonane-6-carboxylate (61 mg, 0.12 mmol, 1.00 equiv) and TFA/DCM (0.5 mL/3 mL) was stirred for 1 h at room temperature. The resulting mixture was concentrated under reduced pressure to give a residue. The residue was purified by reverse flash chromatography (Condition 5, Gradient 8) to afford 1-[2-(7-fluoro-2-methylindazol-5-yl)thieno[2,3-d][1,3]thiazol-5-yl]-1,6-diazaspiro[3.5]nonane (15.30 mg, 31.67%) as a solid. LCMS (ES, m/z):414 [M+H]⁺. ¹H NMR (400 MHz, DMSO-d₆) δ 8.56 (d, J=2.8 Hz, 1H), 8.07 (d, J=1.3 Hz, 1H), 7.58 (dd, J=12.7, 1.4 Hz, 1H), 6.30 (s, 1H), 4.22 (s, 3H), 3.81-3.68 (m, 2H), 2.93 (d, J=11.6 Hz, 1H), 2.80 (dd, J=15.8, 11.0 Hz, 2H), 2.48 (s, 1H), 2.39-2.29 (m, 1H), 2.25 (ddd, J=10.7, 8.3, 6.4 Hz, 1H), 2.11 (td, J=10.3, 9.3, 5.9 Hz, 2H), 1.93-1.81 (m, 1H), 1.60 (d, J=14.2 Hz, 1H).

### Example 40: Synthesis of Compound 203

### Synthesis of Compound 203

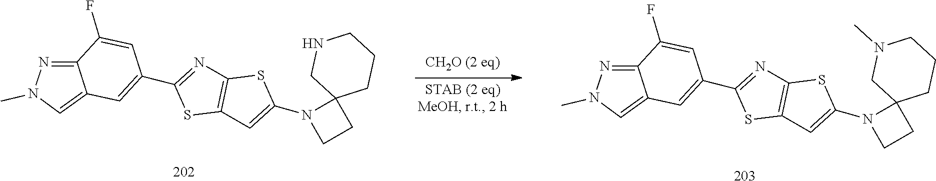 [SMILES: Cn1cc2cc(-c3nc4sc(N5CCC56CCCNC6)cc4s3)cc(F)c2n1 ; CN1CCCC2(CCN2c2cc3sc(-c4cc(F)c5nn(C)cc5c4)nc3s2)C1 ; "202" ; "203"]

A mixture of 1-[2-(7-fluoro-2-methylindazol-5-yl)thieno[2,3-d][1,3]thiazol-5-yl]-1,6-diazaspiro[3.5]nonane (70.00 mg, 0.17 mmol, 1.00 equiv) and HCHO (10.17 mg, 0.34 mmol, 2.00 equiv) in methanol (5 mL) was stirred for 40 min at room temperature. To the reaction mixture was added STAB (71.75 mg, 0.34 mmol, 2.00 equiv). The resulting mixture was stirred for an additional 2 h at room temperature, then extracted with CH₂Cl₂ (3×5 mL). The combined organic layers were washed with saturated NaCl (1×5 mL), dried over anhydrous Na₂SO₄, and filtered. After filtration, the filtrate was concentrated under reduced pressure to give a residue. The residue was purified by reverse flash chromatography (Condition 5, Gradient 8) to afford 1-[2-(7-fluoro-2-methylindazol-5-yl)thieno[2,3-d][1,3]thiazol-5-yl]-6-methyl-1,6-diazaspiro[3.5]nonane (3.40 mg, 4.70%) as a solid. LCMS (ES, m/z):428 [M+H]⁺. ¹H NMR (400 MHz, DMSO-d₆) δ 8.57 (d, J=2.8 Hz, 1H), 8.08 (d, J 1.4 Hz, 1H), 7.58 (dd, J=12.7, 1.4 Hz, 1H), 6.34 (s, 1H), 4.23 (s, 3H), 3.77 (dt, J=8.5, 6.0 Hz, 2H), 2.86 (d, J=10.5 Hz, 1H), 2.20 (s, 5H), 2.11 (q, J=9.8, 8.8 Hz, 1H), 1.78 (s, 4H), 1.71 (dd, J=12.6, 4.3 Hz, 1H), 1.24 (s, 1H).

### Example 41: Synthesis of Compound 201

### Synthesis of Intermediate B102

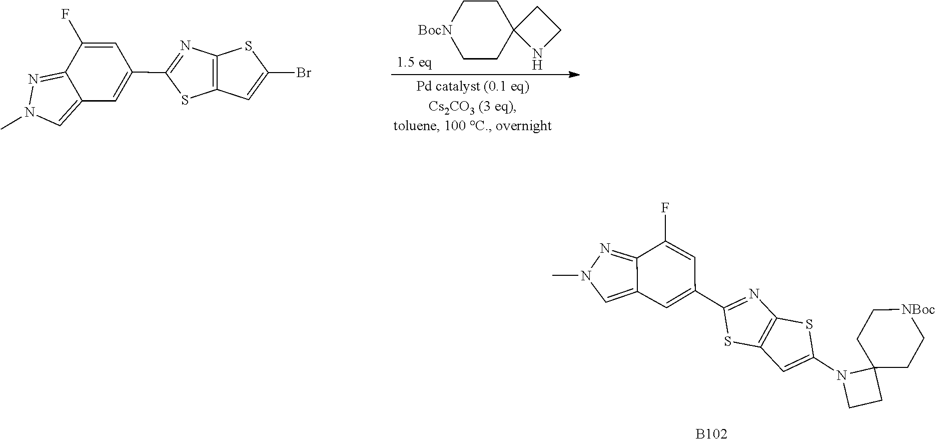 [SMILES: Cn1cc2cc(-c3nc4sc(Br)cc4s3)cc(F)c2n1 ; CC(C)(C)OC(=O)N1CCC2(CCN2)CC1 ; "B102" ; Cn1cc2cc(-c3nc4sc(N5CCC56CCN(C(=O)OC(C)(C)C)CC6)cc4s3)cc(F)c2n1]

To a mixture of 5-{5-bromothieno[2,3-d][1,3]thiazol-2-yl}-7-fluoro-2-methylindazole (200.00 mg, 0.54 mmol, 1.00 equiv) and tert-butyl 1,7-diazaspiro[3.5]nonane-7-carboxylate (184.38 mg, 0.82 mmol, 1.50 equiv) in toluene (5 mL) was added Pd-PEPPSI-IPentCl 2-methylpyridine (o-picoline (45.68 mg, 0.05 mmol, 0.10 equiv) and Cs₂CO₃ (124.06 mg, 1.63 mmol, 3.00 equiv). The reaction mixture was stirred for 8 h at 100° C. under nitrogen atmosphere, then extracted with ethyl acetate (10× mL). The combined organic layers were washed with saturated NaCl (1×10 mL), dried over anhydrous Na₂SO₄, and filtered. After filtration, the filtrate was concentrated under reduced pressure to give a residue. The residue was purified by silica gel column chromatography, eluted with CH₂Cl₂/MeOH (10:1) to afford tert-butyl 1-[2-(7-fluoro-2-methylindazol-5-yl)thieno [2,3-d][1,3]thiazol-5-yl]-1,7-diazaspiro[3.5]nonane-7-carboxylate (190.00 mg, 68.11%) as an oil. LCMS (ES, m/z):514 [M+H]⁺.

### Synthesis of Compound 201

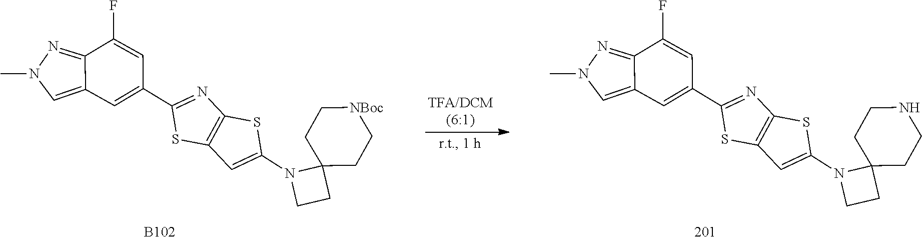 [SMILES: "B102" ; Cn1cc2cc(-c3nc4sc(N5CCC56CCN(C(=O)OC(C)(C)C)CC6)cc4s3)cc(F)c2n1 ; Cn1cc2cc(-c3nc4sc(N5CCC56CCNCC6)cc4s3)cc(F)c2n1 ; "201"]

A mixture of tert-butyl 1-[2-(7-fluoro-2-methylindazol-5-yl)thieno[2,3-d][1,3]thiazol-5-yl]-1,7-diazaspiro[3.5]nonane-7-carboxylate (60.00 mg, 0.12 mmol, 1.00 equiv) and TFA/DCM (0.5 mL/3 mL) was stirred for 1 h at room temperature. The resulting mixture was concentrated under reduced pressure to give a residue. The residue was purified by reverse flash chromatography (Condition 5, Gradient 9) to afford 1-[2-(7-fluoro-2-methylindazol-5-yl)thieno[2,3-d] [1,3]thiazol-5-yl]-1,7-diazaspiro[3.5]nonane (5.90 mg, 12.21%) as a solid. LCMS (ES, m/z):414 [M+H]⁺. ¹H NMR (400 MHz, DMSO-d₆) δ 8.50 (d, J=2.8 Hz, 1H), 8.03 (d, J=1.3 Hz, 1H), 7.53 (dd, J=12.7, 1.4 Hz, 1H), 6.30 (s, 1H), 4.21 (s, 3H), 3.73 (dd, J=7.9, 6.5 Hz, 2H), 3.02 (d, J=11.5 Hz, 2H), 2.93-2.78 (m, 2H), 2.45-2.31 (m, 2H), 2.30-2.20 (m, 2H), 1.86 (ddd, J=13.0, 7.6, 4.9 Hz, 2H).

### Example 42: Synthesis of Compound 204

### Synthesis of Compound 204

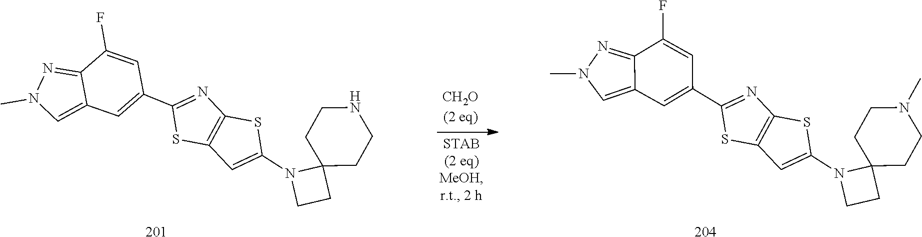 [SMILES: Cn1cc2cc(-c3nc4sc(N5CCC56CCNCC6)cc4s3)cc(F)c2n1 ; CN1CCC2(CC1)CCN2c1cc2sc(-c3cc(F)c4nn(C)cc4c3)nc2s1 ; "201" ; "204"]

A mixture of 1-[2-(7-fluoro-2-methylindazol-5-yl)thieno[2,3-d][1,3]thiazol-5-yl]-1,7-diazaspiro[3.5]nonane (80.00 mg, 0.19 mmol, 1.00 equiv) and HCHO (145.22 mg, 4.83 mmol, 2.00 equiv) in methanol (5 mL) was stirred for 40 min at room temperature. To the reaction mixture was added STAB (82.00 mg, 0.39 mmol, 2.00 equiv). The resulting mixture was stirred for an additional 2 h at room temperature, then extracted with ethyl acetate (3×10 mL). The combined organic layers were washed with saturated NaCl (1×10 mL), dried over anhydrous Na₂SO₄, and filtered. After filtration, the filtrate was concentrated under reduced pressure to give a residue. The residue was purified by reverse flash chromatography (Condition 2, Gradient 6) to afford 1-[2-(7-fluoro-2-methylindazol-5-yl)thieno[2,3-d][1,3]thiazol-5-yl]-7-methyl-1,7-diazaspiro [3.5]nonane (5.70 mg, 6.89%) as a solid. LCMS (ES, m/z):428 [M+H]⁺. ¹H NMR (400 MHz, DMSO-d₆) δ 8.56 (d, J=2.8 Hz, 1H), 8.08 (d, J=1.3 Hz, 1H), 7.58 (dd, J=12.7, 1.4 Hz, 1H), 6.30 (s, 1H), 4.22 (s, 3H), 3.76 (t, J=7.2 Hz, 2H), 2.75 (d, J=11.2 Hz, 2H), 2.15 (d, J=6.3 Hz, 5H), 2.02 (td, J=12.3, 3.8 Hz, 2H), 1.95-1.84 (m, 2H), 1.77-1.69 (m, 2H).

### Example 43: Synthesis of Compound 197

### Synthesis of Intermediate B103

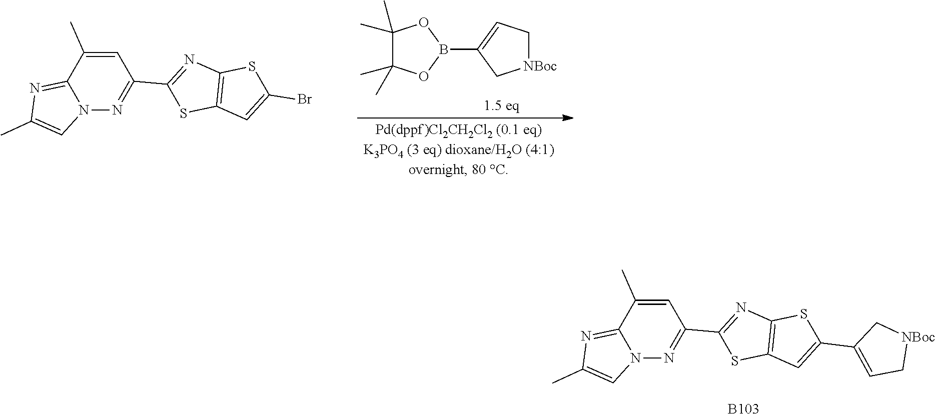 [SMILES: Cc1cn2nc(-c3nc4sc(Br)cc4s3)cc(C)c2n1 ; CC(C)(C)OC(=O)N1CC=C(B2OC(C)(C)C(C)(C)O2)C1 ; "B103" ; Cc1cn2nc(-c3nc4sc(C5=CCN(C(=O)OC(C)(C)C)C5)cc4s3)cc(C)c2n1]

To a mixture of 6-{5-bromothieno[2,3-d][1,3]thiazol-2-yl}-2,8-dimethylimidazo[1,2-b]pyridazine (80.0 mg, 0.22 mmol, 1.00 equiv) and tert-butyl 3-(4,4,5,5-tetramethyl-1,3,2-dioxaborolan-2-yl)-2,5-dihydropyrrole-1-carboxylate (96.98 mg, 0.33 mmol, 1.50 equiv) in a mixture of dioxane/water (3 mL) was added Pd(dppf)Cl₂CH₂Cl₂ (17.84 mg, 0.02 mmol, 0.10 equiv) and K₃PO₄ (139.47 mg, 0.66 mmol, 3.00 equiv). The reaction mixture was stirred overnight at 80° C. under nitrogen atmosphere. The resulting mixture was extracted with ethyl acetate (3×10 mL). The combined organic layers were washed with saturated NaCl (1×10 mL), dried over anhydrous Na₂SO₄, and filtered. After filtration, the filtrate was concentrated under reduced pressure to give a residue. The residue was purified by silica gel column chromatography, eluted with PE/EA (5:1) to afford tert-butyl 3-(2-{2,8-dimethylimidazo [1,2-b]pyridazin-6-yl}thieno[2,3-d][1,3]thiazol-5-yl)-2,5-dihydropyrrole-1-carboxylate (40.00 mg, 40.27%) as a solid. LCMS (ES, m/z):454 [M+H]⁺.

### Synthesis of Intermediate B104

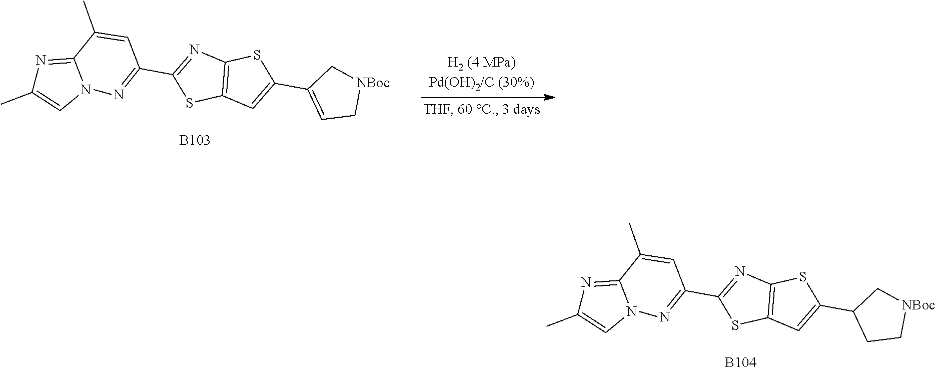 [SMILES: "B103" ; Cc1cn2nc(-c3nc4sc(C5=CCN(C(=O)OC(C)(C)C)C5)cc4s3)cc(C)c2n1 ; "B104" ; Cc1cn2nc(-c3nc4sc(C5CCN(C(=O)OC(C)(C)C)C5)cc4s3)cc(C)c2n1]

To a mixture of tert-butyl 3-(2-{2,8-dimethylimidazo[1,2-b]pyridazin-6-yl}thieno [2,3-d][1,3]thiazol-5-yl)-2,5-dihydropyrrole-1-carboxylate (40.00 mg, 0.09 mmol, 1.00 equiv) and Pd(OH)₂/C (10.00 mg, 0.07 mmol, 0.81 equiv) in THF (5 mL) was added H₂ (4 Mpa). The reaction mixture was stirred for 3 days at 60° C. The resulting mixture was filtered, and the filter cake was washed with THF (3×2 mL). The filtrate was concentrated under reduced pressure to afford tert-butyl 3-(2-{2,8-dimethylimidazo [1,2-b]pyridazin-6-yl}thieno[2,3-d][1,3]thiazol-5-yl)pyrrolidine-1-carboxylate (30.00 mg, 74.67%) as a solid. LCMS (ES, m/z):456 [M+H]⁺.

### Synthesis of Compound 197

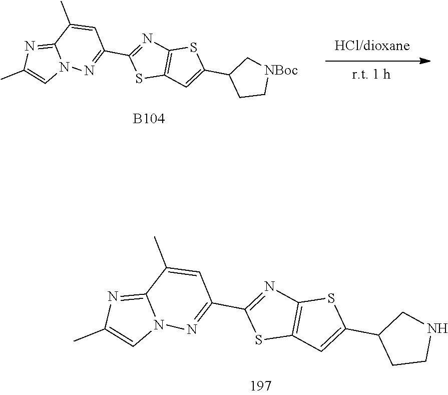 [SMILES: "B104" ; Cc1cn2nc(-c3nc4sc(C5CCN(C(=O)OC(C)(C)C)C5)cc4s3)cc(C)c2n1 ; Cc1cn2nc(-c3nc4sc(C5CCNC5)cc4s3)cc(C)c2n1]

A solution of tert-butyl 3-(2-{2,8-dimethylimidazo[1,2-b]pyridazin-6-yl}thieno [2,3-d][1,3]thiazol-5-yl)pyrrolidine-1-carboxylate (30.00 mg, 0.07 mmol, 1.00 equiv) and HCl (gas) in 1,4-dioxane (5 mL) was stirred for 1 h at room temperature. The resulting mixture was concentrated under reduced pressure to give a residue. The residue was purified by reverse flash chromatography (Condition 5, Gradient 10) to afford 3-(2-{2,8-dimethylimidazo[1,2-b]pyridazin-6-yl}thieno [2,3-d][1,3]thiazol-5-yl)pyrrolidine (3.80 mg, 16.23%) as a solid. LCMS (ES, m/z):356 [M+H]⁺. ¹H NMR (400 MHz, DMSO-d₆) δ 8.15 (d, J=3.8 Hz, 1H), 7.83 (s, 1H), 7.38 (s, 1H), 3.79 (d, J=6.1 Hz, 1H), 3.55 (p, J=7.7 Hz, 1H), 3.27 (dd, J=10.6, 7.3 Hz, 1H), 2.87 (m, 1H), 2.77 (dd, J=10.6, 7.0 Hz, 1H), 2.63 (s, 3H), 2.42 (s, 3H), 2.28-2.21 (m, 1H), 1.80 (dq, J=12.7, 7.5 Hz, 1H).

### Example 44: Synthesis of Compound 198

### Synthesis of Intermediate B105

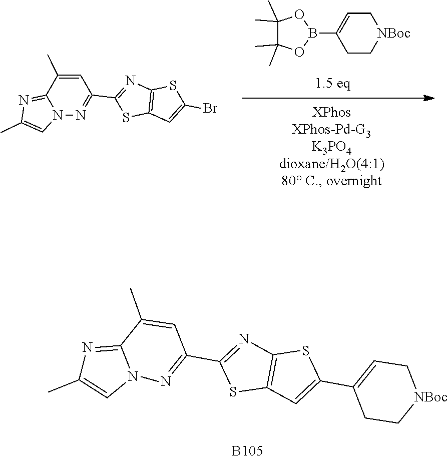 [SMILES: Cc1cn2nc(-c3nc4sc(Br)cc4s3)cc(C)c2n1 ; CC(C)(C)OC(=O)N1CC=C(B2OC(C)(C)C(C)(C)O2)CC1 ; "B105" ; Cc1cn2nc(-c3nc4sc(C5=CCN(C(=O)OC(C)(C)C)CC5)cc4s3)cc(C)c2n1]

To a mixture of 6-{5-bromothieno[2,3-d][1,3]thiazol-2-yl}-2,8-dimethylimidazo[1,2-b]pyridazine (200.00 mg, 0.55 mmol, 1.00 equiv) and tert-butyl 4-(4,4,5,5-tetramethyl-1,3,2-dioxaborolan-2-yl)-3,6-dihydro-2H-pyridine-1-carboxylate (253.96 mg, 0.82 mmol, 1.50 equiv) in a mixture of dioxane/water (5 mL) was added XPhos Pd G₃ (46.35 mg, 0.06 mmol, 0.10 equiv), XPhos (52.20 mg, 0.11 mmol, 0.20 equiv), and K₃PO₄ (348.67 mg, 1.64 mmol, 3.00 equiv). The reaction mixture was stirred overnight at 80° C. under nitrogen atmosphere, then extracted with ethyl acetate (3×10 mL). The combined organic layers were washed with saturated NaCl (1×10 mL), dried over anhydrous Na₂SO₄, and filtered. After filtration, the filtrate was concentrated under reduced pressure to give a residue. The residue was purified by silica gel column chromatography, eluted with CH₂Cl₂/MeOH (10:1) to afford tert-butyl 4-(2-{2,8-dimethylimidazo[1,2-b]pyridazin-6-yl}thieno [2,3-d][1,3]thiazol-5-yl)-3,6-dihydro-2H-pyridine-1-carboxylate (83.00 mg, 32.42%) as a solid. LCMS (ES, m/z):468 [M+H]⁺.

### Synthesis of Compound 198

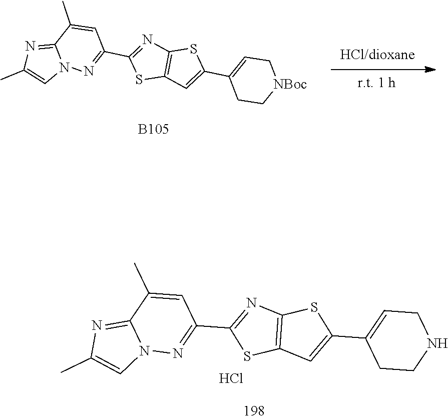 [SMILES: "B105" ; Cc1cn2nc(-c3nc4sc(C5=CCN(C(=O)OC(C)(C)C)CC5)cc4s3)cc(C)c2n1 ; Cc1cn2nc(-c3nc4sc(C5=CCNCC5)cc4s3)cc(C)c2n1 ; Cl]

A mixture of tert-butyl 4-(2-{2,8-dimethylimidazo[1,2-b]pyridazin-6-yl}thieno [2,3-d][1,3]thiazol-5-yl)-3,6-dihydro-2H-pyridine-1-carboxylate (63.00 mg, 0.14 mmol, 1.00 equiv) and HCl (gas) in 1,4-dioxane (5 mL) was stirred for 1 h at room temperature. The resulting mixture was concentrated under reduced pressure to give a residue. The residue was purified by reverse flash chromatography (Condition 8, Gradient 1) to afford 4-(2-{2,8-dimethylimidazo[1,2-b]pyridazin-6-yl} thieno[2,3-d][1,3]thiazol-5-yl)-1,2, 3,6-tetrahydropyridine (6.90 mg, 13.94%) as a solid. LCMS (ES, m/z):368 [M+H]⁺. ¹H NMR (400 MHz, DMSO-d₆) δ 8.95 (s, 2H), 8.20 (s, 1H), 7.89 (s, 1H), 7.67 (s, 1H), 6.29 (s, 1H), 3.81 (s, 2H), 3.31 (s, 2H), 2.77 (s, 2H), 2.65 (d, J=1.1 Hz, 3H), 2.44 (s, 3H).

### Example 45: Synthesis of Compounds 110, 155, 156, 157, 158, 163, 164, 165, 174, 179, 180

### Synthesis of Intermediate B106

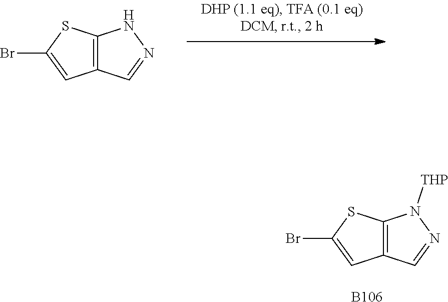

To a stirred mixture of 5-bromo-1H-thieno[2,3-c] pyrazole (6 g, 29.54 mmol, 1.00 equiv) and DHP (4.97 g, 59.09 mmol, 2 equiv) in DCM (60 mL, 943.80 mmol, 31.94 equiv) was added TFA (2 mL, 26.92 mmol, 0.91 equiv) portionwise at room temperature under nitrogen atmosphere. The resulting mixture was stirred for 2 h at room temperature under nitrogen atmosphere, then basified to pH 7 with saturated NaHCO₃. The resulting mixture was diluted with water (50 mL) and extracted with ethyl acetate (2×60 mL). The combined organic layers were washed with brine (2×100 mL), dried over anhydrous Na₂SO₄, and filtered. After filtration, the filtrate was concentrated under reduced pressure to afford 5-bromo-1-(oxan-2-yl) thieno[2,3-c] pyrazole (8 g, 94%) as an oil.

### Synthesis of Intermediate B107

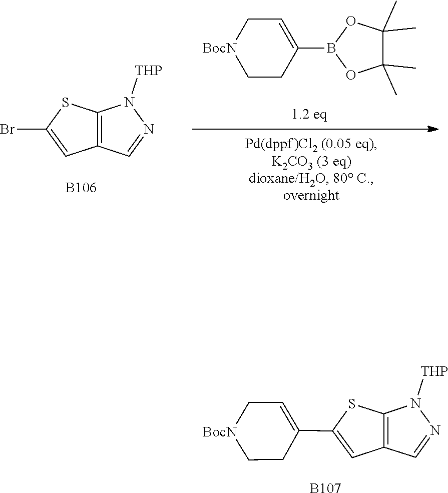

To a stirred mixture of 5-bromo-1-(oxan-2-yl)thieno[2,3-c]pyrazole (8 g, 27.85 mmol, 1.0 equiv) and tert-butyl 4-(4,4,5,5-tetramethyl-1,3,2-dioxaborolan-2-yl)-3,6-dihydro-2H-pyridine-1-carboxylate (12.92 g, 41.78 mmol, 1.5 equiv) in dioxane (80 mL) was added Pd(dppf)Cl₂CH₂Cl₂ (1.13 g, 1.39 mmol, 0.05 equiv), K₂CO₃ (11.55 g, 83.57 mmol, 3 equiv), and water (15 mL) portionwise at room temperature under nitrogen atmosphere. The resulting mixture was stirred overnight at 80° C. under nitrogen atmosphere. The resulting mixture was diluted with water (80 mL) and extracted with ethyl acetate (2×100 mL). The combined organic layers were washed with brine (2×100 mL), dried over anhydrous Na₂SO₄, and filtered. After filtration, the filtrate was concentrated under reduced pressure to give a residue. The residue was purified by silica gel column chromatography, eluted with CH₂Cl₂/MeOH (1:1) to afford tert-butyl 4-[1-(oxan-2-yl) thieno[2,3-c]pyrazol-5-yl]-3,6-dihydro-2H-pyridine-1-carboxylate (8 g, 73.73%) as a solid.

### Synthesis of Intermediate B108

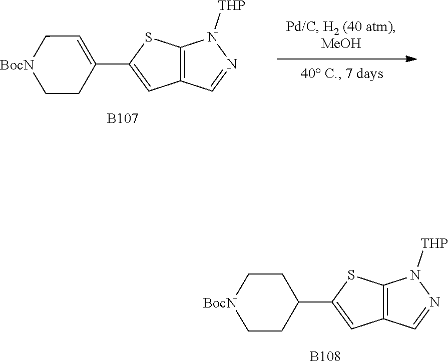

To a stirred solution of tert-butyl 4-[1-(oxan-2-yl) thieno[2,3-c] pyrazol-5-yl]-3,6-dihydro-2H-pyridine-1-carboxylate (8 g, 20.53 mmol, 1.00 equiv) in methanol was added Pd/C (8 g, 21.26 mmol, 1.04 equiv) portionwise at room temperature under hydrogen atmosphere. The resulting mixture was stirred for 7 days at 40° C. under hydrogen atmosphere (40 atm), then filtered, and the filter cake washed with methanol (2×20 mL). The filtrate was concentrated under reduced pressure to afford tert-butyl 4-[1-(oxan-2-yl)thieno[2,3-c]pyrazol-5-yl]piperidine-1-carboxylate (7 g, 87.05%) as a solid.

### Synthesis of Intermediate B109

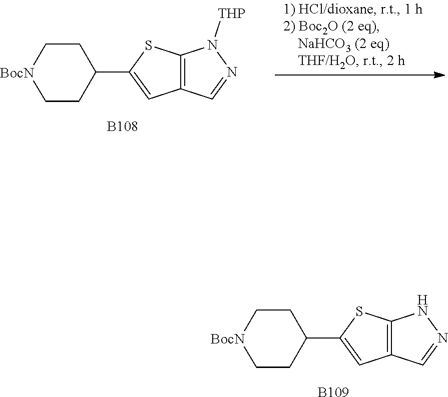

To a stirred solution of tert-butyl 4-[1-(oxan-2-yl) thieno[2,3-c] pyrazol-5-yl] piperidine-1-carboxylate (6 g, 15.324 mmol, 1.00 equiv) was added HCl (gas) in 1,4-dioxane (60 mL, 1974.71 mmol, 128.86 equiv) portionwise at room temperature under nitrogen atmosphere. The resulting mixture was stirred for 1 h at room temperature under nitrogen atmosphere, then filtered. After filtration, the filtrate was concentrated under reduced pressure to give a residue. To the residue was added NaHCO₃ (2.57 g, 30.648 mmol, 2 equiv) in water (50 mL, 2775.41 mmol, 181.11 equiv), and the mixture was stirred for 10 min. To the resulting mixture was added THE (50 mL, 617.150 mmol, 40.27 equiv) and (Boc)₂O (3.68 g, 16.85 mmol, 1.1 equiv) portionwise at room temperature under nitrogen atmosphere, and the reaction mixture was stirred for 2 h at room temperature under nitrogen atmosphere, then extracted with ethyl acetate (2×50 mL). The combined organic layers were washed with brine (2×50 mL), dried over anhydrous Na₂SO₄, and filtered. After filtration, the filtrate was concentrated under reduced pressure to give a residue. The residue was purified by silica gel column chromatography, eluted with PE/EA (1:1) to afford tert-butyl 4-{1H-thieno[2,3-c]pyrazol-5-yl}piperidine-1-carboxylate (2.8 g, 59.44%) as a solid.

### Synthesis of Intermediate B110

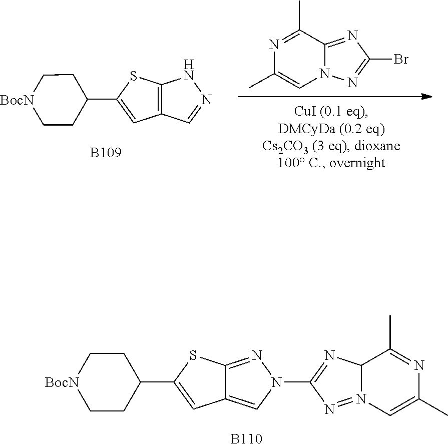

A mixture of tert-butyl 4-{1H-thieno[2,3-c]pyrazol-5-yl}piperidine-1-carboxylate (200 mg, 0.651 mmol, 1.00 equiv), 2-bromo-6,8-dimethyl-[1,2,4]triazolo[1,5-a]pyrazine (177.27 mg, 0.781 mmol, 1.2 equiv), CuI (12.39 mg, 0.065 mmol, 0.1 equiv), (1R,2R)-1-N,2-N-dimethylcyclohexane-1,2-diamine (18.51 mg, 0.130 mmol, 0.2 equiv), and Cs₂CO₃ (635.93 mg, 1.953 mmol, 3 equiv) in dioxane (10 mL, 118.041 mmol, 181.43 equiv) was stirred for 1 days at 100° C. under nitrogen atmosphere. The resulting mixture was concentrated under reduced pressure to give a residue. The residue was purified by silica gel column chromatography, eluted with PE/EtOAc (1:5) to yield a solid. The solid was further purified by Prep-HPLC (Column: YMC-Actus Triart C18, 30×150 mm, 5 m; Mobile Phase A: Water (10 mmol/L NH₄HCO₃), Mobile Phase B: ACN; Flow rate: 60 mL/min; Gradient: 5% B to 50% B in 8 min; Wave Length: 220 nm; RT1(min): 6.23.), followed by Chiral-Prep-HPLC (Column: CHIRALPAK IA, 2×25 cm, 5 m; Mobile Phase A: Hex(0.1% DEA)-HPLC, Mobile Phase B: EtOH-HPLC; Flow rate: 20 mL/min; Gradient: 50% B to 50% B in 14 min; Wave Length: 220/254 nm; RT1(min): 7.2; RT2(min): 12.1; Sample Solvent: DCM−HPLC; Injection Volume: 0.4 mL; Number Of Runs: 8; Column temperature: room temperature) to afford tert-butyl 4-(2-{6,8-dimethyl-[1,2,4]triazolo[1,5-a]pyrazin-2-yl} thieno[2,3-c]pyrazol-5-yl)piperidine-1-carboxylate (40 mg, 13.56%) as a solid.

### Synthesis of Compound 163

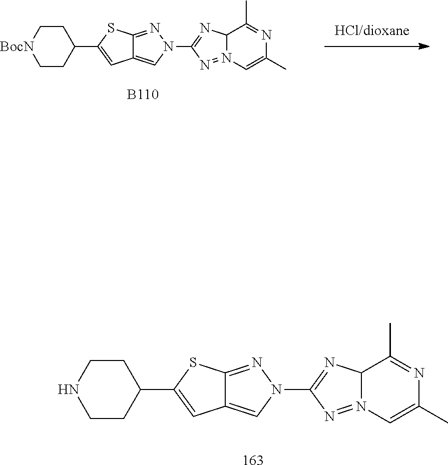

To a stirred solution of tert-butyl 4-(2-{6,8-dimethyl-[1,2,4]triazolo[1,5-a]pyrazin-2-yl}thieno[2,3-c] pyrazol-5-yl)piperidine-1-carboxylate (40 mg, 0.088 mmol, 1.00 equiv) in dioxane (2 mL, 23.608 mmol, 267.70 equiv) was added HCl (gas) in 1,4-dioxane (2 mL, 65.8 mmol, 746 equiv) dropwise at room temperature under air atmosphere. The resulting mixture was stirred for 5 h at room temperature under air atmosphere, then concentrated under vacuum to give a residue. The residue was purified by Prep-HIPLC (Column: YMC-Actus Triart C18, 30×150 mm, 5 m; Mobile Phase A: Water (10 mmol/L NH₄HCO₃), Mobile Phase B: ACN; Flow rate: 60 mL/min; Gradient: 5% B to 45% B in 8 min; Wave Length: 220 nm; RT1(min): 6.22) to afford 4-(2-{6,8-dimethyl-[1,2,4]triazolo [1,5-a]pyrazin-2-yl} thieno[2,3-c]pyrazol-5-yl)piperidine (21 mg, 67.37=) as a solid.

Compounds 110, 155, 156, 157, 158, 163, 164, 165, 174, 179, and 180 were prepared according to the procedures described herein and outlined in this Example 45. The table below provides intermediates used in these procedures and final compound characterization data.

| | | LCMS | |
|---|---|---|---|
| | | (ESI, | |
| | | m/z) | |
| Compound No. and Structure | Coupling Reagent | [M + H]⁺ | ¹H NMR δ |
| 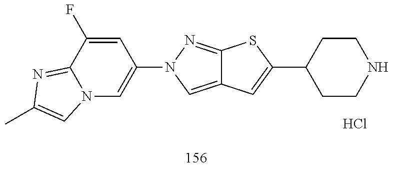 [SMILES: Cc1cn2cc(-n3cc4cc(C5CCNCC5)sc4n3)cc(F)c2n1 ; Cl] | 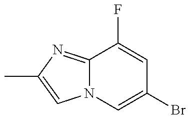 | 356 | (400 MHz, DMSO-d₆) δ 9.25 (s, 1H), 8.95 (d, J = 10.5 Hz, 1H), 8.77 (d, J = 11.0 Hz, 1H), 8.71 (s, 1H), 8.23- 8.11 (m, 1H), 8.07 (d, J = 2.7 Hz, 1H), 6.95 (s, 1H), 3.36 (d, J = 12.5 Hz, 2H), 3.18 (td, J = 11.6, 9.8, 5.7 Hz, |
| | | | 1H), 3.02 (q, J = 11.9 |
| | | | Hz, 2H), 2.45 (s, 3H), |
| | | | 2.16 (d, J = 12.8 Hz, |
| | | | 2H), 1.88 (qd, J = |
| | | | 13.0, 4.0 Hz, 2H) |
| 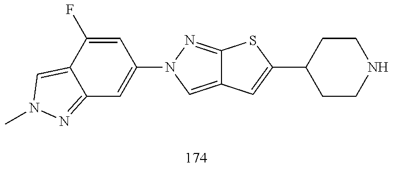 [SMILES: Cn1cc2c(F)cc(-n3cc4cc(C5CCNCC5)sc4n3)cc2n1] | 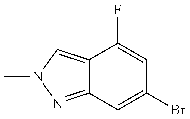 | 356 | (400 MHz, DMSO-d₆) δ 8.73 (s, 1H), 8.61 (s, 1H), 7.93 (t, J = 1.3 Hz, 1H), 7.52 (dd, J = 11.9, 1.6 Hz, 1H), 6.82 (d, J = 1.2 Hz, 1H), 4.20 (s, 3H), 3.06- 2.97 (m, 2H), 2.93- 2.83 (m, 1H), 2.59 (td, J = 12.1, 2.4 Hz, 2H), |
| | | | 1.91 (d, J = 12.5 Hz, |
| | | | 2H), 1.54 (qd, J = |
| | | | 12.2, 4.0 Hz, 2H) |
| 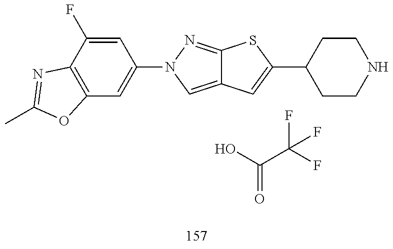 | 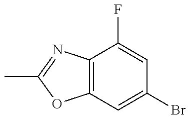 | 357 | (400 MHz, DMSO-d₆) δ 8.02 (d, J = 2.9 Hz, 1H), 7.77 (s, 1H), 7.55 (d, J = 11.0 Hz, 1H), 6.95 (s, 1H), 3.06 (d, J = 12.5 Hz, 2H), 2.97 (d, J = 12.8 Hz, 1H), 2.64 (s, 5H), 2.03- 1.95 (m, 2H), 1.65 (d, J = 14.6 Hz, 1H), 1.59 (d, J = 12.3 Hz, 1H) |
| 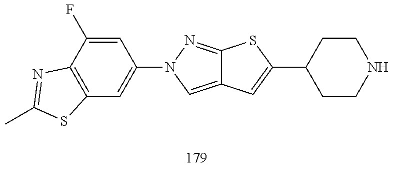 [SMILES: Cc1nc2c(F)cc(-n3cc4cc(C5CCNCC5)sc4n3)cc2s1] | 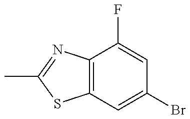 | 373 | (400 MHz, DMSO-d₆) δ 8.80 (d, J = 42.3 Hz, 1H), 8.52 (d, J = 32.1 Hz, 1H), 8.25 (d, J = 2.1 Hz, 1H), 8.12 (s, 1H), 7.76 (dd, J = 11.7, 2.1 Hz, 1H), 7.06 (d, J = 1.1 Hz, 1H), 3.40 (s, 2H), 3.23 (d, J = 11.1 Hz, 1H), 3.05 |
| | | | (t, J = 12.6 Hz, 2H), |
| | | | 2.86 (s, 3H), 2.20 (d, J = |
| | | | 13.6 Hz, 2H), 1.87 |
| | | | (q, J = 12.2, 11.7 Hz, |
| | | | 2H) |
| 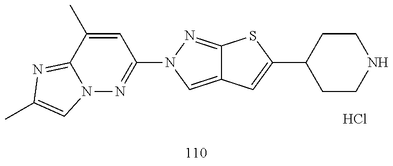 [SMILES: Cc1cn2nc(-n3cc4cc(C5CCNCC5)sc4n3)cc(C)c2n1 ; Cl] | 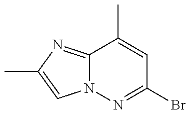 | 353 | (400 MHz, DMSO-d₆) δ 8.97 (d, J = 11.6 Hz, 1H), 8.80 (d, J = 10.8 Hz, 1H), 8.75 (s, 1H), 8.25 (s, 1H), 8.09 (s, 1H), 6.92 (s, 1H), 3.37 (d, J = 12.5 Hz, 2H), 3.25-3.14 (m, 1H), 3.05 (d, J = 11.9 Hz, 1H), 2.99 (d, J = 12.0 |
| | | | Hz, 1H), 2.71 (s, 3H), |
| | | | 2.49 (s, 3H), 2.21- |
| | | | 2.11 (m, 2H), 1.92 (dd, |
| | | | J = 12.7, 3.9 Hz, 1H), |
| | | | 1.86 (dd, J = 13.0, 3.7 |
| | | | Hz, 1H) |
| 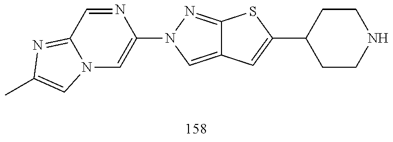 [SMILES: Cc1cn2cc(-n3cc4cc(C5CCNCC5)sc4n3)ncc2n1] | 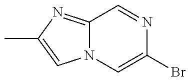 | 339 | (400 MHz, DMSO-d₆) δ 9.13 (s, 1H), 8.96 (s, 1H), 8.08 (s, 1H), 8.00 (s, 1H), 6.88 (s, 1H), 3.11-3.03 (m, 2H), 2.91 (d, J = 11.6 Hz, 1H), 2.66 (s, 2H), 2.44 (s, 3H), 1.96 (d, J = 12.5 Hz, 2H), 1.58 (p, |
| | | | J = 13.3, 12.1 Hz, 2H) |
| 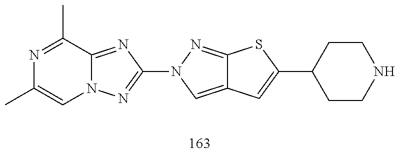 [SMILES: Cc1cn2nc(-n3cc4cc(C5CCNCC5)sc4n3)nc2c(C)n1] | 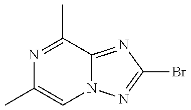 | 354 | (400 MHz, 353K, DMSO-d₆): δ 8.72- 8.67 (m, 1H), 8.55 (s, 1H), 6.79 (d, J = 1.2 Hz, 1H), 3.02 (s, 2H), 2.92-2.86 (m, 1H), 2.80 (s, 3H), 2.67- 2.56 (m, 2H), 2.52 (d, J = 0.9 Hz, 3H), 1.98- 1.88 (m, 2H), 1.63- |
| | | | 1.49 (m, 2H) |
| 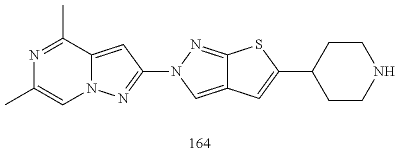 [SMILES: Cc1cn2nc(-n3cc4cc(C5CCNCC5)sc4n3)cc2c(C)n1] | 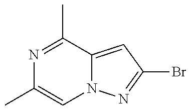 | 353 | (400 MHz, 353K, DMSO-d₆): δ 8.43 (s, 1H), 8.00 (s, 1H), 7.05 (s, 1H), 6.90 (d, J = 1.1 Hz, 1H), 3.08 (s, 2H), 2.98-2.91 (m, 1H), 2.71 (s, 3H), 2.65 (dd, J = 12.2, 2.6 Hz, 2H), 2.44 (s, 3H), 2.02- 1.93 (m, 2H), 1.60 (qd, |
| | | | J = 12.1, 4.0 Hz, 2H) |
| 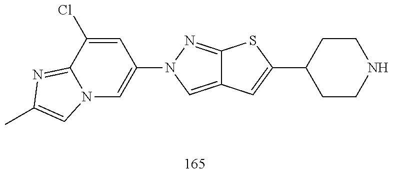 [SMILES: Cc1cn2cc(-n3cc4cc(C5CCNCC5)sc4n3)cc(Cl)c2n1] | 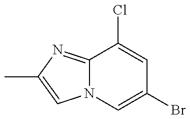 | 372 | (400 MHz, DMSO-d₆): δ 9.13 (d, J = 1.9 Hz, 1H), 8.59 (s, 1H), 8.03 (t, J = 2.1 Hz, 1H), 7.89 (s, 1H), 6.81 (s, 1H), 3.01 (d, J = 12.2 Hz, 2H), 2.92-2.79 (m, 1H), 2.58 (t, J = 11.8 Hz, 2H), 2.38 (s, 3H), 1.90 (dt, J = 13.0, 6.9 Hz, 2H), 1.52 (qd, J = |
| | | | 12.2, 3.9 Hz, 2H) |
| 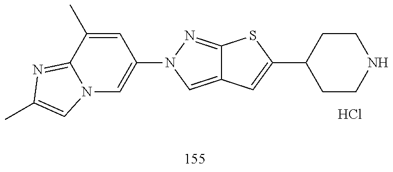 [SMILES: Cc1cn2cc(-n3cc4cc(C5CCNCC5)sc4n3)cc(C)c2n1 ; Cl] | 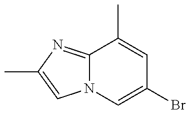 | 352 | (400 MHz, DMSO-d₆) δ 9.00 (s, 1H), 8.09 (dd, J = 13.9, 7.9 Hz, 1H), 8.02 (s, 2H), 7.06- 7.00 (m, 1H), 3.37 (dd, J = 12.9, 7.2 Hz, 2H), 3.19 (s, 1H), 2.99 (d, J = 15.6 Hz, 2H), 2.62 (d, J = 5.4 Hz, 3H), 2.46 (d, J = 5.3 Hz, 3H), 2.19 (d, J = |
| | | | 14.0 Hz, 2H), 1.82 (d, |
| | | | J = 12.7 Hz, 2H) |
| 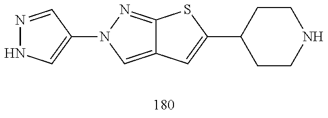 [SMILES: c1n[nH]cc1-n1cc2cc(C3CCNCC3)sc2n1] | 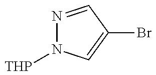 | 274 | (400 MHz, DMSO-d₆) δ 8.30 (s, 1H), 8.08 (s, 2H), 6.79 (s, 1H), 3.01 (d, J = 12.0 Hz, 1H), 2.87 (d, J = 12.5 Hz, 1H), 2.57 (dd, J = 23.0, 10.7 Hz, 2H), 1.90 (d, J = 12.6 Hz, |
| | | | 2H), 1.52 (qd, J = |
| | | | 12.2, 4.0 Hz, 2H) |
| 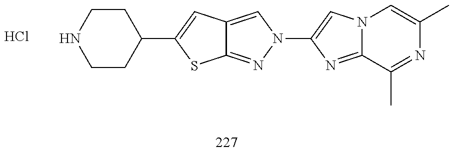 [SMILES: Cl ; Cc1cn2cc(-n3cc4cc(C5CCNCC5)sc4n3)nc2c(C)n1] | 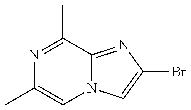 | 353 | (400 MHz, DMSO-d₆) δ 9.16 (s, 1H), 8.94 (s, 1H), 8.39 (s, 1H), 8.30 (s, 1H), 8.02 (s, 1H), 6.97 (d, J = 1.1 Hz, 1H), 3.46-3.34 (m, 2H), 3.24 (td, J = 11.6, 5.8 Hz, 1H), 3.11- 3.02 (m, J = 12.4 Hz, 2H), 2.83 (s, 3H), 2.47 |
| | | | (d, J = 1.0 Hz, 3H), |
| | | | 2.26-2.17 (m, 2H), |
| | | | 2.07-1.92 (m, 2H) |
| 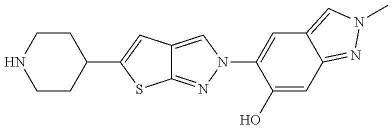 [SMILES: Cn1cc2cc(-n3cc4cc(C5CCNCC5)sc4n3)c(O)cc2n1] | 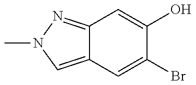 | 354 | (400 MHz, DMSO-d₆) δ1H NMR (400 MHz, DMSO-d6) δ 8.27 (s, 2H), 7.89 (s, 1H), 7.04 (s, 1H), 6.79 (s, 1H), 4.11 (s, 3H), 3.05 (s, 2H), 2.90 (s, 1H), 2.65 (d, J = 15.1 Hz, 2H), 1.93 (d, J = 12.7 Hz, |
| | | | 2H), 1.59-1.53 (dd, J = |
| | | | 12.2, 3.9 Hz, 2H) |

### Example 46: Synthesis of Compounds 177, 182, 183, 184, 186, 187, 190

### Synthesis Intermediate B111

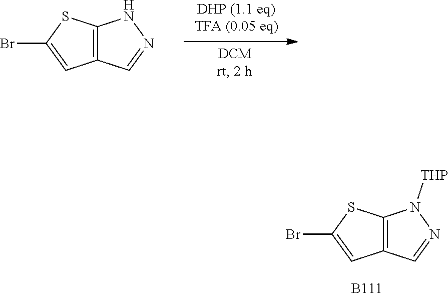

A solution of 5-bromo-1H-thieno[2,3-c] pyrazole (2.00 g, 9.36 mmol, 1.00 equiv) in DCM (20 mL) was treated at 25° C. with DHP (0.91 g, 10.29 mmol, 1.10 equiv), added over the course of 5 minutes under nitrogen atmosphere. To the reaction mixture was added TFA (0.06 g, 0.47 mmol, 0.05 equiv) dropwise, and the reaction mixture was stirring for 2 h at 25° C. The resulting mixture was extracted with ethyl acetate (2×50 mL). The combined organic layers were washed with saturated salt solution (50 mL), dried over anhydrous Na₂SO₄, and filtered. After filtration, the filtrate was concentrated under reduced pressure to afford B111 (2.80 g) as a solid.

### Synthesis of Intermediate B112

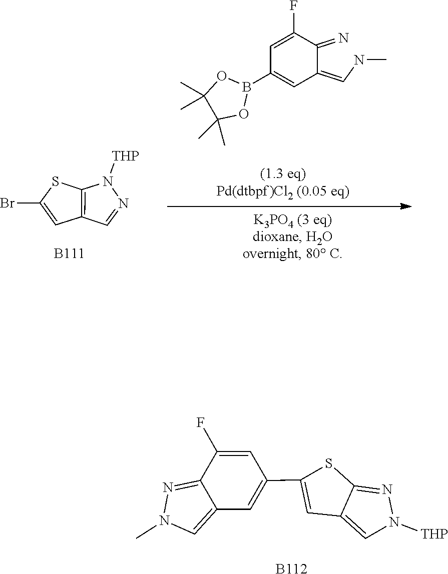

To a stirred solution of 5-bromo-1-(oxan-2-yl) thieno[2,3-c]pyrazole (1.90 g, 6.62 mmol, 1.00 equiv) and 7-fluoro-2-methyl-5-(4,4,5,5-tetramethyl-1,3,2-dioxaborolan-2-yl)indazole (2.37 g, 8.60 mmol, 1.30 equiv) in 1,4-dioxane (19 mL) and H₂O (3.80 mL) was added Pd(dtbpf)Cl₂ (0.43 g, 0.66 mmol, 0.10 equiv) and K₃PO₄ (4.21 g, 19.85 mmol, 3.00 equiv) at 100° C. under N₂ atmosphere. The reaction mixture was stirred overnight at 80° C., then extracted with ethyl acetate (2×50 mL). The combined organic layers were washed with saturated salt solution (50 mL), dried over anhydrous Na₂SO₄, and filtered. After filtration, the filtrate was concentrated under reduced pressure to give a residue. The residue was purified by silica gel column chromatography, eluted with EA and PE (5:2) to afford 7-fluoro-2-methyl-5-[2-(oxan-2-yl) thieno[2,3-c] pyrazol-5-yl] indazole (1.8 g) as a solid.

### Synthesis of Intermediate B113

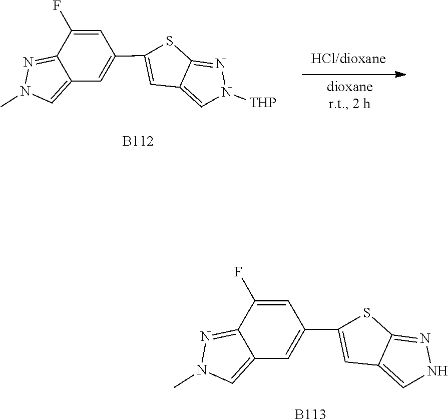

To a stirred solution of 7-fluoro-2-methyl-5-[2-(oxan-2-yl) thieno[2,3-c] pyrazol-5-yl] indazole (1.8 g, 5.050 mmol, 1.00 equiv) in dioxane (18 mL) was added HCl (gas) in 1,4-dioxane (18 mL, 592.414 mmol, 117.30 equiv) portionwise at room temperature under nitrogen atmosphere. The resulting mixture was stirred for 2 h at room temperature under nitrogen atmosphere, then filtered. After filtration, the filtrate was concentrated under reduced pressure to afford 7-fluoro-2-methyl-5-{2H-thieno[2,3-c]pyrazol-5-yl}indazole (2 g, 145.44%) as a solid.

### Synthesis of Intermediate B114

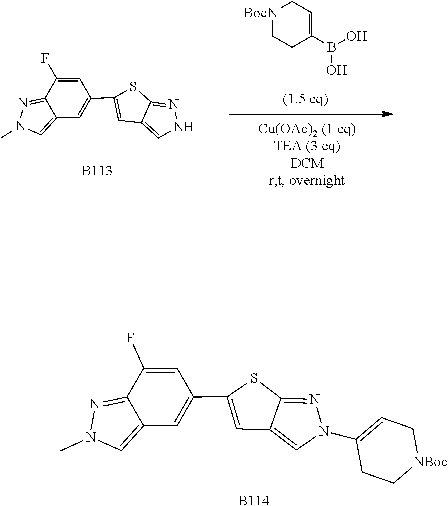 [SMILES: "B113" ; "B114" ; Cn1cc2cc(-c3cc4c[nH]nc4s3)cc(F)c2n1 ; Cn1cc2cc(-c3cc4cn(C5=CCN(C(=O)OC(C)(C)C)CC5)nc4s3)cc(F)c2n1 ; CC(C)(C)OC(=O)N1CC=C(B(O)O)CC1]

To a stirred solution of 7-fluoro-2-methyl-5-{2H-thieno[2,3-c] pyrazol-5-yl} indazole (200 mg, 0.734 mmol, 1.00 equiv) and 1-(tert-butoxycarbonyl)-3,6-dihydro-2H-pyridin-4-ylboronic acid (250.17 mg, 1.101 mmol, 1.5 equiv) in DCM (2 mL) was added Cu(OAc)₂ (133.41 mg, 0.734 mmol, 1 equiv) and TEA (222.97 mg, 2.202 mmol, 3 equiv) at room temperature under nitrogen atmosphere. The resulting mixture was stirred overnight at room temperature under nitrogen atmosphere, then concentrated in vacuo to give a residue. The residue was purified by silica gel column chromatography, eluted with PE/EA (5:1) followed by chiral HPLC (to afford tert-butyl 4-[5-(7-fluoro-2-methylindazol-5-yl)thieno[2,3-c]pyrazol-2-yl]-3,6-dihydro-2H-pyridine-1-carboxylate (25 mg) as a solid.

### Synthesis of Intermediate B115

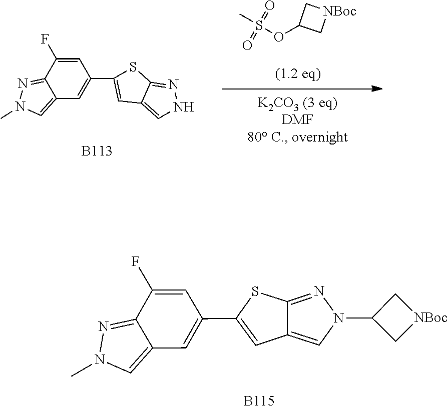

To a stirred mixture of 7-fluoro-2-methyl-5-{2H-thieno[2,3-c]pyrazol-5-yl}indazole (200 mg, 0.734 mmol, 1.00 equiv) and tert-butyl 3-(methanesulfonyloxy)azetidine-1-carboxylate (221.49 mg, 0.881 mmol, 1.20 equiv) in DMF (2 ml) was added K₂CO₃ (304.53 mg, 2.202 mmol, 3 equiv) at 80° C. under nitrogen atmosphere. The resulting mixture was stirred overnight at 80° C. under nitrogen atmosphere, then concentrated in vacuo to give a residue. The residue was purified by silica gel column chromatography, eluted with PE/EA (5:1) to afford tert-butyl 3-[5-(7-fluoro-2-methylindazol-5-yl) thieno[2,3-c] pyrazol-2-yl] azetidine-1-carboxylate (20 mg) as a solids and tert-butyl 3-[5-(7-fluoro-2-methylindazol-5-yl) thieno [2,3-c] pyrazol-1-yl]azetidine-1l-carboxylate (25 mg) as a solid.

Compounds 177, 182, 183, 184, 186, 187, and 190 were prepared according to the procedures described herein and outlined in this Example 46. The table below provides intermediates used in these procedures and final compound characterization data.

| | | LCMS | |
|---|---|---|---|
| | | (ESI, | |
| | Coupling | m/z) | |
| Compound No. and Structure | Reagent | [M + H]⁺ | ¹H NMR δ |
| 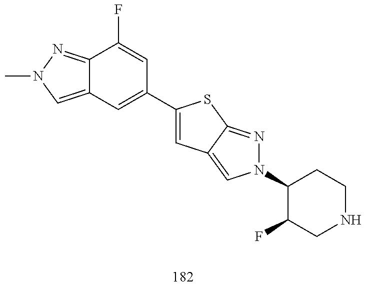 [SMILES: Cn1cc2cc(-c3cc4cn([C@H]5CCNC[C@H]5F)nc4s3)cc(F)c2n1] | 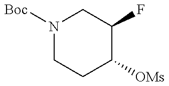 [SMILES: CC(C)(C)OC(=O)N1CC[C@@H](OS(C)(=O)=O)[C@H](F)C1] | 374 | (400 MHz, DMSO-d₆) δ 8.50 (d, J = 2.8 Hz, 1H), 8.16 (s, 1H), 7.72 (d, J = 1.3 Hz, 1H), 7.52 (d, J = 1.5 Hz, 1H), 7.49 (s, 1H), 4.83 (ddt, J = 50.7, 9.9, 5.0 Hz, 1H), 4.51 (d, J = 9.9 Hz, 1H), 4.21 (s, 3H), 3.34 (s, 1H), 2.97 (dd, J = 13.0, 3.5 Hz, 1H), 2.51 (p, J = 1.9 Hz, 2H), 2.05 (h, J = 5.4, 4.7 Hz, 2H) |
| 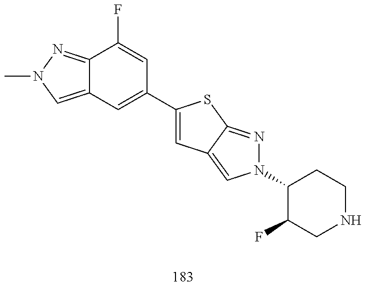 [SMILES: Cn1cc2cc(-c3cc4cn([C@@H]5CCNC[C@H]5F)nc4s3)cc(F)c2n1] | 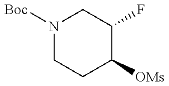 [SMILES: CC(C)(C)OC(=O)N1CC[C@H](OS(C)(=O)=O)[C@@H](F)C1] | 374 | (400 MHz, DMSO-d₆) δ 8.50 (d, J = 2.8 Hz, 1H), 8.16 (s, 1H), 7.72 (d, J = 1.3 Hz, 1H), 7.53 (dd, J = 13.0, 1.4 Hz, 1H), 7.49 (s, 1H), 4.84 (dtd, J = 50.7, 9.8, 5.2 Hz, 1H), 4.52 (qd, J = 10.3, 6.5 Hz, 1H), 4.21 (s, 3H), 3.34 (s, 1H), 3.03-2.91 (m, 1H), 2.57 (ddd, J = 14.7, 10.5, 4.5 Hz, 2H), 2.14-1.97 (m, 2H) |
| 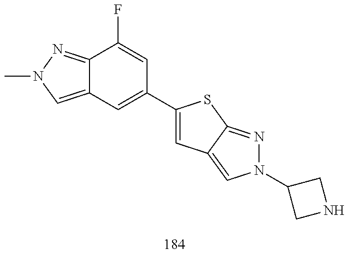 [SMILES: Cn1cc2cc(-c3cc4cn(C5CNC5)nc4s3)cc(F)c2n1] | 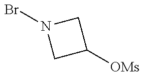 | 328 | (400 MHz, DMSO-d₆) δ 8.44 (d, J = 2.9 Hz, 1H), 8.12 (s, 1H), 7.73 (d, J = 1.5 Hz, 1H), 7.41 (d, J = 13.9 Hz, 2H), 5.34 (d, J = 7.9 Hz, 1H), 4.22 (s, 3H), 3.98 (d, J = 48.6 Hz, 4H) |
| 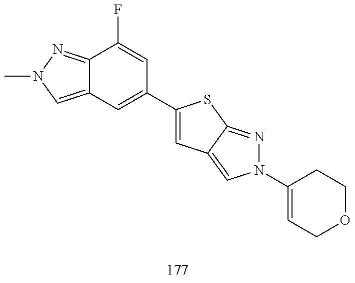 [SMILES: Cn1cc2cc(-c3cc4cn(C5=CCOCC5)nc4s3)cc(F)c2n1] | 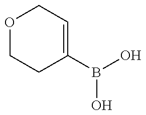 | 355 | (400 MHz, DMSO-d₆) δ 8.51 (d, J = 2.8 Hz, 1H), 8.38 (s, 1H), 7.75 (d, J = 1.4 Hz, 1H), 7.61-7.56 (m, 1H), 7.53 (s, 1H), 6.38-6.30 (m, 1H), 4.28 (q, J = 2.8 Hz, 2H), 4.21 (s, 3H), 3.90 (t, J = 5.5 Hz, 2H), 2.74 (tt, J = 5.8, 2.9 Hz, 2H) |
| 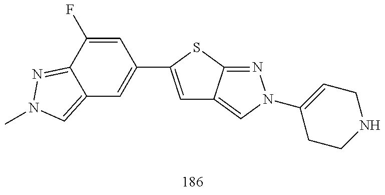 [SMILES: Cn1cc2cc(-c3cc4cn(C5=CCNCC5)nc4s3)cc(F)c2n1] | 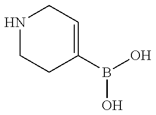 | 354 | (400 MHz, DMSO-d₆) δ 8.50 (s, 1H), 8.33 (s, 1H), 7.74 (s, 1H), 7.57 (d, J = 13.0 Hz, 1H), 7.51 (s, 1H), 6.31 (s, 1H), 4.21 (s, 3H), 3.40 (s, 2H), 2.97 (d, J = 5.8 Hz, 2H), 2.59 (s, 2H) |
| 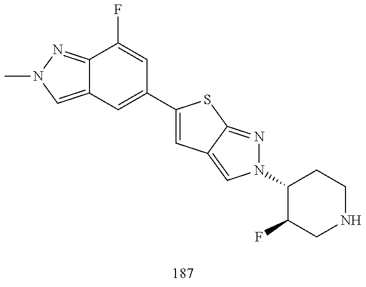 [SMILES: Cn1cc2cc(-c3cc4cn([C@@H]5CCNC[C@H]5F)nc4s3)cc(F)c2n1] | 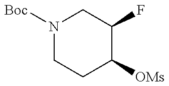 [SMILES: CC(C)(C)OC(=O)N1CC[C@H](OS(C)(=O)=O)[C@H](F)C1] | 374 | (400 MHz, DMSO-d₆) δ 10.09 (d, J = 11.1 Hz, 1H), 9.02 (d, J = 11.4 Hz, 1H), 8.51 (d, J = 2.7 Hz, 1H), 8.16 (s, 1H), 7.74 (d, J = 1.3 Hz, 1H), 7.58-7.49 (m, 2H), 5.38 (d, J = 47.7 Hz, 1H), 5.08-4.89 (m, 1H), 4.21 (s, 3H), 3.52- 3.36 (m, 2H), 3.21 (d, J = 11.9 Hz, 1H), 2.64- 2.54 (m, 1H), 2.34 (dd, J = 13.8, 4.1 Hz, 1H) |
| 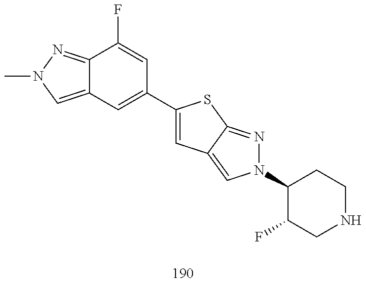 [SMILES: Cn1cc2cc(-c3cc4cn([C@H]5CCNC[C@@H]5F)nc4s3)cc(F)c2n1] | 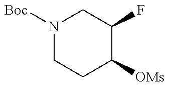 [SMILES: CC(C)(C)OC(=O)N1CC[C@H](OS(C)(=O)=O)[C@H](F)C1] | 374 | (400 MHz, DMSO-d6) δ 8.50 (d, J = 2.8 Hz, 1H), 8.09 (d, J = 0.9 Hz, 1H), 7.73 (d, J = 1.4 Hz, 1H), 7.56 (d, J = 1.5 Hz, 1H), 7.49 (s, 1H), 4.96 (d, J = 50.7 Hz, 1H), 4.68 (dddd, J = 30.6, 12.7, 4.4, 2.0 Hz, 1H), 4.21 (s, 3H), 3.25-3.17 (m, 1H), 3.15-3.04 (m, 1H), 2.96-2.81 (m, 1H), 2.76-2.64 (m, 1H), 2.36-2.16 (m, 1H), 1.98 (dd, J = 12.7, 3.9 Hz, 1H) |

### Example 47: Synthesis of Compound 230

### Synthesis of Compound 230

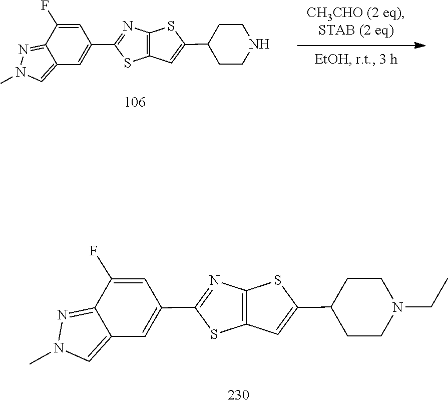 [SMILES: Cn1cc2cc(-c3nc4sc(C5CCNCC5)cc4s3)cc(F)c2n1 ; CCN1CCC(c2cc3sc(-c4cc(F)c5nn(C)cc5c4)nc3s2)CC1]

A mixture of 7-fluoro-2-methyl-5-[5-(piperidin-4-yl)thieno[2,3-d][1,3]thiazol-2-yl]indazole (14 mg, 0.04 mmol, 1.00 equiv) and 2-oxoacetic acid hydrate (6.92 mg, 0.08 mmol, 2.00 equiv) in ethanol (2 mL) was stirred for 1 h at room temperature, To the reaction mixture was added STAB (15.93 mg, 0.076 mmol, 2 equiv) and the resulting mixture was stirred for an additional 2 h at room temperature. The resulting mixture was extracted with ethyl acetate (2×5 mL). The combined organic layers were washed with sat. NaCl (1×2 mL), dried over anhydrous Na₂SO₄, and filtered. After filtration, the filtrate was concentrated under reduced pressure to give a residue. The residue was purified by reverse flash chromatography (Column: YMC-Actus Triart C18, 30*150 mm, 5 m; Mobile Phase A: Water(10 mmol/L NH₄HCO₃), Mobile Phase B: ACN; Flow rate: 60 mL/min; Gradient: 40% B to 80% B in 8 min, 80% B; Wave Length: 220 nm; RT1(min): 6.03) to afford 5-[5-(1-ethylpiperidin-4-yl)thieno[2,3-d][1,3]thiazol-2-yl]-7-fluoro-2-methylindazole (2 mg, 13.29%) as a solid. LCMS (ES, m/z):401 [M+H]⁺. ¹H NMR (400 MHz, DMSO-d₆) δ 8.62 (d, J=2.7 Hz, 1H), 8.22 (d, J=1.3 Hz, 1H), 7.65 (dd, J=12.5, 1.4 Hz, 1H), 7.31 (d, J=1.0 Hz, 1H), 4.24 (s, 3H), 3.00 (m, 2H), 2.85 (m, 1H), 2.40-2.32 (m, 2H), 2.05-1.94 (m, 4H), 1.68 (qd, J=13.0, 4.0 Hz, 2H), 1.02 (t, J=7.2 Hz, 3H).

### Example 48: Synthesis of Compound 231

### Synthesis of Intermediate B116

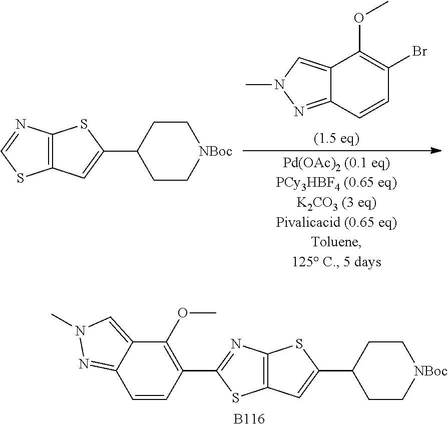

To a stirred mixture of tert-butyl 4-{thieno[2,3-d][1,3]thiazol-5-yl}piperidine-1-carboxylate (100 mg, 0.31 mmol, 1.00 equiv), 5-bromo-4-methoxy-2-methylindazole (111.46 mg, 0.46 mmol, 1.50 equiv), Pd(OAc)₂ (6.92 mg, 0.03 mmol, 0.10 equiv), and K₂CO₃ (127.79 mg, 0.92 mmol, 3.00 equiv) in toluene (5 mL) was added PCy₃HBF₄ (70.37 mg, 0.19 mmol, 0.65 equiv) and pivalic acid (20.46 mg, 0.20 mmol, 0.65 equiv). The reaction mixture was stirred for 5 days at 125° C. under nitrogen atmosphere, then extracted with ethyl acetate (3×10 mL). The combined organic layers were washed with sat. NaCl (1×10 mL), dried over anhydrous Na₂SO₄, and filtered. After filtration, the filtrate was concentrated under reduced pressure to give a residue. The residue was purified by silica gel column chromatography, eluted with PE/EA (1:1) to afford tert-butyl 4-[2-(4-methoxy-2-methylindazol-5-yl)thieno[2,3-d][1,3]thiazol-5-yl]piperidine-1-carboxylate (60 mg, 40.17%) as a solid. LCMS (ES, m/z):485 [M+H]⁺.

### Synthesis of Compound 231

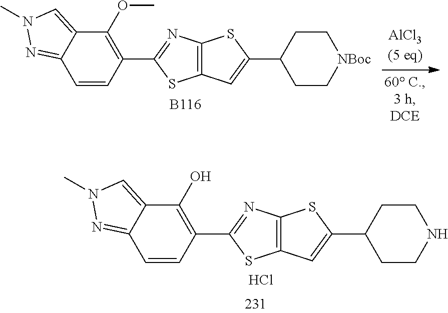 [SMILES: "B116" ; COc1c(-c2nc3sc(C4CCN(C(=O)OC(C)(C)C)CC4)cc3s2)ccc2nn(C)cc12 ; Cn1cc2c(O)c(-c3nc4sc(C5CCNCC5)cc4s3)ccc2n1 ; Cl]

A mixture of tert-butyl 4-[2-(4-methoxy-2-methylindazol-5-yl)thieno[2,3-d][1,3]thiazol-5-yl]piperidine-1-carboxylate (50 mg, 0.10 mmol, 1.00 equiv) and AlCl₃ (13.76 mg, 0.10 mmol, 1.00 equiv) in DCE (5 mL) was stirred for 3 h at 60° C. The reaction mixture was quenched with water (1 mL) at room temperature, then extracted with ethyl acetate (2×10 mL). The combined organic layers were washed with sat. NaCl (1×5 mL), dried over anhydrous Na₂SO₄, and filtered. After filtration, the filtrate was concentrated under reduced pressure to give a residue. The residue was purified by reverse flash chromatography (Column: Xselect CSH OBD Column 30*150 mm Sum, n; Mobile Phase A: Water (0.05% HCl), Mobile Phase B: ACN; Flow rate: 60 mL/min; Gradient: 3% B to 18% B in 8 min, 18% B; Wave Length: 210 nm; RT1(min): 5.53) to afford 2-methyl-5-[5-(piperidin-4-yl) thieno[2,3-d][1,3]thiazol-2-yl]indazol-4-ol (3.50 mg, 9.16%) as a solid. LCMS (ES, m/z):371 [M+H]⁺. ¹H NMR (400 MHz, DMSO-d₆) δ 12.08 (s, 1H), 8.97 (d, J=11.3 Hz, 1H), 8.76-8.68 (m, 1H), 8.57 (s, 1H), 7.92 (d, J=9.1 Hz, 1H), 7.32 (d, J=14.9 Hz, 1H), 7.21 (d, 1H), 4.16 (s, 3H), 3.37 (d, J=12.5 Hz, 2H), 3.29-3.22 (m, 1H), 3.06 (d, J=12.2 Hz, 2H), 2.18 (d, J=13.4 Hz, 2H), 1.89 (qd, J=13.1, 3.9 Hz, 2H).

### Example 49: Synthesis of Compound 235

### Synthesis of Intermediate B117

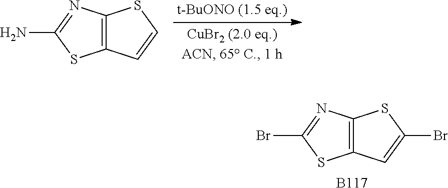 [SMILES: Nc1nc2sccc2s1 ; "B117" ; Brc1cc2sc(Br)nc2s1]

In a 3-necked round-bottom flask, t-BuONO (8.91 g, 86.42 mmol, 1.50 equiv), CuBr₂ (25.74 g, 115.22 mmol, 2.00 equiv), and ACN (270 mL) were combined at room temperature. The reaction mixture was stirred for 15 min at 65° C. To the resulting mixture was added thieno[2,3-d] [1,3] thiazol-2-amine (9.00 g, 57.61 mmol, 1.00 equiv), and the reaction mixture was stirred for an additional 1 h. The resulting mixture was filtered, the filter cake was washed with DCM, and the filtrate was concentrated under reduced pressure to give a residue. The residue was purified by silica gel column chromatography, eluted with PE/EA (1:1) to afford 2,5-dibromothieno[2,3-d] [1,3] thiazole (6.7 g, 38.90%) as a solid. LCMS (ES, m/z):298 [M+H]⁺.

### Synthesis of Intermediate B118

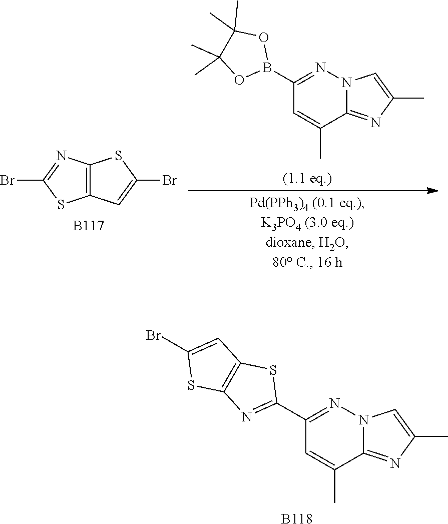

To a solution of 2,5-dibromothieno[2,3-d] [1,3] thiazole (600 mg, 2.01 mmol, 1.00 equiv) and 2,8-dimethyl-6-(4,4,5,5-tetramethyl-1,3,2-dioxaborolan-2-yl) imidazo[1,2-b] pyridazine (602.92 mg, 2.21 mmol, 1.10 equiv) in dioxane (12 mL) and H₂O (3 mL) was added K₃PO₄ (1277.86 mg, 6.02 mmol, 3.00 equiv) and Pd(PPh₃)₄(231.88 mg, 0.20 mmol, 0.10 equiv). After stirring for 16 h at 80° C. under a nitrogen atmosphere, the resulting mixture was concentrated under reduced pressure to give a residue. The residue was purified by Prep-TLC/silica gel column chromatography, eluted with PE/EA (1:1) to afford 6-{5-bromothieno[2,3-d] [1,3] thiazol-2-yl}-2,8-dimethylimidazo[1,2-b] pyridazine (140 mg, 19.10%) as a solid. LCMS (ES, m/z):365 [M+H]⁺.

### Synthesis of Intermediate B119

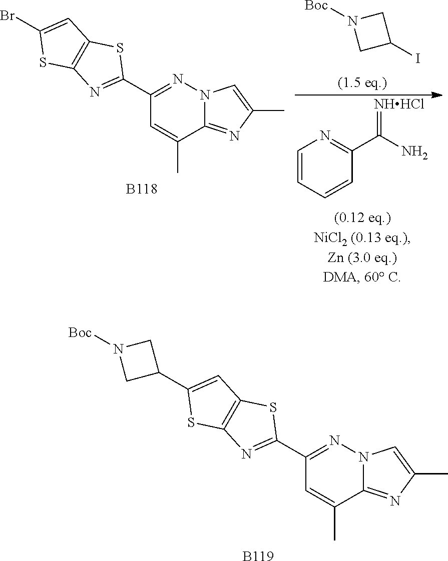

A mixture of tert-butyl 3-iodoazetidine-1-carboxylate (58.13 mg, 0.20 mmol, 1.50 equiv), pyridine-2-carboximidamide (1.99 mg, 0.02 mmol, 0.12 equiv), and NiCl₂ (2.31 mg, 0.02 mmol, 0.13 equiv) in DMA (2 mL) was stirred for 1 h at 40° C. under nitrogen atmosphere. To the reaction mixture was added 6-{5-bromothieno[2,3-d] [1,3] thiazol-2-yl}-2,8-dimethylimidazo[1,2-b] pyridazine (50 mg, 0.14 mmol, 1.00 equiv) and Zn (26.86 mg, 0.41 mmol, 3.00 equiv) portionwise at 60° C. The reaction mixture was concentrated in vacuo to give a residue. The residue was purified by silica gel column chromatography, eluted with PE/EA (5:1) to afford tert-butyl 3-(2-{2,8-dimethylimidazo[1,2-b] pyridazin-6-yl} thieno[2,3-d][1,3] thiazol-5-yl) azetidine-1-carboxylate (15 mg, 24.82%) as a solid. LCMS (ES, m/z):442 [M+H]⁺.

### Synthesis of Compound 235

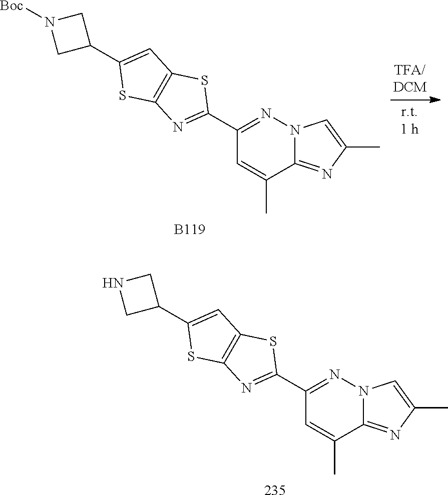 [SMILES: "B119" ; Cc1cn2nc(-c3nc4sc(C5CN(C(=O)OC(C)(C)C)C5)cc4s3)cc(C)c2n1 ; Cc1cn2nc(-c3nc4sc(C5CNC5)cc4s3)cc(C)c2n1 ; "235"]

A mixture of tert-butyl 3-(2-{2,8-dimethylimidazo[1,2-b] pyridazin-6-yl} thieno [2,3-d] [1,3]thiazol-5-yl) azetidine-1-carboxylate (13 mg, 0.03 mmol, 1.00 equiv), DCM (0.6 mL), and TFA (0.10 mL) was stirred for 1 h at room temperature. The reaction mixture was concentrated in vacuo to give a residue. The residue was purified by Chiral-Prep-HPLC (Column, XBridge Prep OBD C18 Column, 30*150 mm, 5 pm; mobile phase, Water (10 mmol/L NH₄HCO₃) and ACN (5% ACN up to 40% in 8 min)) to afford 3-(2-{2,8-dimethylimidazo[1,2-b] pyridazin-6-yl} thieno[2,3-d] [1,3] thiazol-5-yl) azetidine (3.4 mg, 33.69%) as a solid. LCMS (ES, m/z):342 [M+H]⁺. ¹H NMR (400 MHz, DMSO-d₆) δ 8.17-8.12 (m, 1H), 7.85 (d, J=1.2 Hz, 1H), 7.43 (d, J=0.9 Hz, 1H), 4.17 (p, J=7.4 Hz, 1H), 3.85 (t, J=7.6 Hz, 2H), 3.61 (t, J=7.0 Hz, 2H), 2.64 (d, J=1.2 Hz, 3H), 2.42 (s, 3H).

### Example 50: Synthesis of Compound 232

### Synthesis of Intermediate B120

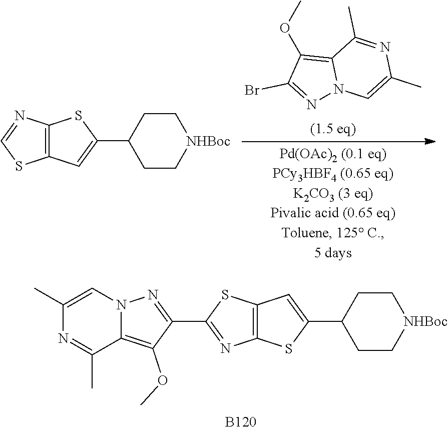

To a stirred mixture of tert-butyl 4-{thieno[2,3-d][1,3]thiazol-5-yl}piperidine-1-carboxylate (100.00 mg, 0.31 mmol, 1.00 equiv), 2-bromo-3-methoxy-4,6-dimethylpyrazolo[1,5-a]pyrazine (118.40 mg, 0.46 mmol, 1.50 equiv), Pd(OAc)₂ (6.92 mg, 0.03 mmol, 0.10 equiv), and K₂CO₃ (127.79 mg, 0.92 mmol, 3.00 equiv) in toluene (5 mL) was added PCy₃HBF₄ (56.18 mg, 0.20 mmol, 0.65 equiv) and pivalic acid (20.46 mg, 0.20 mmol, 0.65 equiv). The reaction mixture was stirred for 5 days at 125° C. under nitrogen atmosphere, then extracted with ethyl acetate (3×10 mL). The combined organic layers were washed with sat. NaCl (1×10 mL), dried over anhydrous Na₂SO₄, and filtered. After filtration, the filtrate was concentrated under reduced pressure to give a residue. The residue was purified by silica gel column chromatography, eluted with PE/EA (1:1) to afford tert-butyl 4-(2-{3-methoxy-4,6-dimethylpyrazolo[1,5-a]pyrazin-2-yl}thieno [2,3-d][1,3] thiazol-5-yl)piperidine-1-carboxylate (55.00 mg, 35.72%) as a solid. LCMS (ES, m/z):500 [M+H]⁺.

### Synthesis of Compound 232

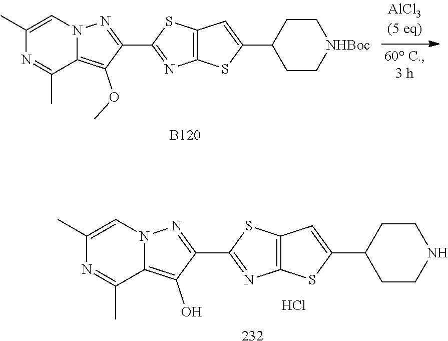

A mixture of tert-butyl 4-(2-{3-methoxy-4,6-dimethylpyrazolo[1,5-a]pyrazin-2-yl}thieno[2,3-d][1,3] thiazol-5-yl)piperidine-1-carboxylate (50.00 mg, 0.10 mmol, 1.00 equiv) and AlCl₃ (66.72 mg, 0.50 mmol, 5.00 equiv) in DCE (5 mL) was stirred for 3 h at 60° C. The reaction mixture was quenched with water (1 mL) at room temperature. The resulting mixture was extracted with ethyl acetate (2×10 mL). The combined organic layers were washed with sat. NaCl (1×10 mL), dried over anhydrous Na₂SO₄, and filtered. After filtration, the filtrate was concentrated under reduced pressure to give a residue. The residue was purified by reverse flash chromatography (Column: Xselect CSH OBD Column 30*150 mm Sum, n; Mobile Phase A: Water(0.05% HCl), Mobile Phase B: ACN; Flow rate: 60 mL/min; Gradient: 3% B to 18% B in 8 min, 18% B; Wave Length: 210 nm; RT1(min): 5.53) to afford 4,6-dimethyl-2-[5-(piperidin-4-yl)thieno[2,3-d][1,3]thiazol-2-yl]pyrazolo[1,5-a] pyrazin-3-ol hydrochloride (2.00 mg, 4.74%) as a solid. LCMS (ES, m/z):386 [M+H]⁺. ¹H NMR (400 MHz, DMSO-d₆) δ 9.88 (s, 1H), 8.97 (d, J=11.3 Hz, 1H), 8.76 (d, J=10.5 Hz, 1H), 8.42 (s, 1H), 7.38 (s, 1H), 3.38 (d, J=12.6 Hz, 2H), 3.07 (d, J=12.0 Hz, 1H), 3.01 (d, J=12.2 Hz, 2H), 2.83 (s, 3H), 2.40 (s, 3H), 2.19 (d, J=13.8 Hz, 2H), 1.97-1.82 (m, 2H).

### Example 51: Synthesis of Compound 239

### Synthesis of Intermediate B121

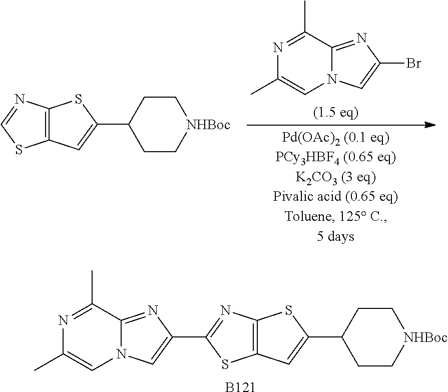

To a stirred mixture of tert-butyl 4-{thieno[2,3-d][1,3]thiazol-5-yl}piperidine-1-carboxylate (100 mg, 0.31 mmol, 1.00 equiv), 2-bromo-6,8-dimethylimidazo[1,2-a]pyrazine (104.52 mg, 0.46 mmol, 1.50 equiv), Pd(OAc)₂ (6.92 mg, 0.03 mmol, 0.10 equiv) and PCy₃HBF₄ (73.77 mg, 0.20 mmol, 0.65 equiv) in toluene (5 mL) was added pivalic acid (20.46 mg, 0.20 mmol, 0.65 equiv) and K₂CO₃ (127.79 mg, 0.92 mmol, 3.00 equiv). The reaction mixture was stirred for 5 days at 125° C. under nitrogen atmosphere. The resulting mixture was extracted with ethyl acetate (3×10 mL). The combined organic layers were washed with sat. NaCl (1×10 mL), dried over anhydrous Na₂SO₄, and filtered. After filtration, the filtrate was concentrated under reduced pressure to give a residue. The residue was purified by silica gel column chromatography, eluted with PE/EA (1:1) to afford tert-butyl 4-(2-{6,8-dimethylimidazo[1,2-a]pyrazin-2-yl}thieno[2,3-d][1,3]thiazol-5-yl)piperidine-1-carboxylate (30 mg, 20.73%) as a solid. LCMS (ES, m/z):470 [M+H]⁺.

### Synthesis of Compound 239

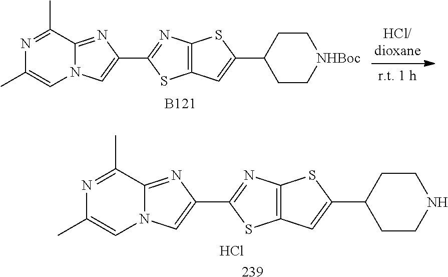

A mixture of tert-butyl 4-(2-{6,8-dimethylimidazo[1,2-a]pyrazin-2-yl}thieno[2,3-d][1,3]thiazol-5-yl)piperidine-1-carboxylate (30 mg, 0.06 mmol, 1.00 equiv) and HCl (gas) in 1,4-dioxane (5 mL) was stirred for 1 h at room temperature. The resulting mixture was concentrated under reduced pressure to give a residue. The residue was purified by reverse flash chromatography (Column: Xselect CSH C18 OBD Column 30*150 mm 5 μm, n; Mobile Phase A: Water (0.05% HCl), Mobile Phase B: ACN; Flow rate: 60 mL/min; Gradient: 3% B to 3% B in 2 min, 3% B to 28% B in 8 min, 28% B; Wave Length: 220 nm; RT1(min): 6.85) to afford 4-(2-{6,8-dimethylimidazo[1,2-a]pyrazin-2-yl}thieno[2,3-d][1,3]thiazol-5-yl)piperidine (5.50 mg, 23.30%) as a solid. LCMS (ES, m/z):370 [M+H]⁺. ¹H NMR (400 MHz, DMSO-d₆) δ 8.61 (s, 1H), 8.34 (s, 1H), 7.37 (s, 1H), 3.40 (d, J=13.7 Hz, 2H), 3.31 (s, 1H), 3.06 (t, J=12.4 Hz, 2H), 2.80 (s, 3H), 2.42 (s, 3H), 2.27-2.19 (m, 2H), 1.90 (d, J=12.8 Hz, 2H).

### Example 52: Synthesis of Compound 233

### Synthesis of Intermediate B122

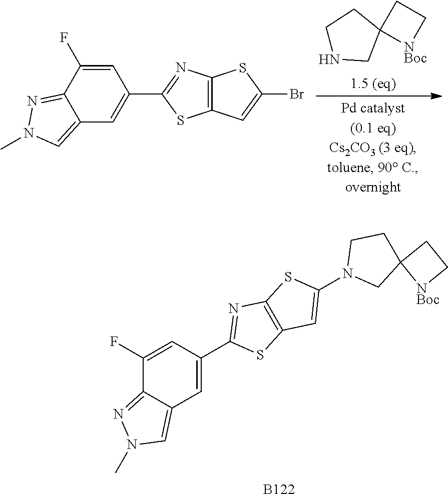 [SMILES: Cn1cc2cc(-c3nc4sc(Br)cc4s3)cc(F)c2n1 ; CC(C)(C)OC(=O)N1CCC12CCNC2 ; "B122" ; Cn1cc2cc(-c3nc4sc(N5CCC6(CCN6C(=O)OC(C)(C)C)C5)cc4s3)cc(F)c2n1]

To a stirred mixture of 5-{5-bromothieno[2,3-d][1,3]thiazol-2-yl}-7-fluoro-2-methylindazole (100 mg, 0.27 mmol, 1.00 equiv) and tert-butyl 1,6-diazaspiro[3.4]octane-1-carboxylate (69.18 mg, 0.33 mmol, 1.20 equiv) in toluene (3 mL) was added Pd-PEPPSI-IPentCl 2-methylpyridine (o-picoline) (22.84 mg, 0.03 mmol, 0.10 equiv) and Cs₂CO₃ (265.44 mg, 0.82 mmol, 3.00 equiv). The reaction mixture was stirred overnight at 90° C. under nitrogen atmosphere, then extracted with ethyl acetate (3×10 mL). The combined organic layers were washed with sat. NaCl (1×10 mL), dried over anhydrous Na₂SO₄, and filtered. After filtration, the filtrate was concentrated under reduced pressure to give a residue. The residue was purified by silica gel column chromatography, eluted with PE/EA (5:1) to afford tert-butyl 6-[2-(7-fluoro-2-methylindazol-5-yl)thieno [2,3-d][1,3]thiazol-5-yl]-1,6-diazaspiro[3.4]octane-1-carboxylate (60 mg, 44.22%) as a solid. LCMS (ES, m/z):500 [M+H]⁺.

### Synthesis of Compound 233

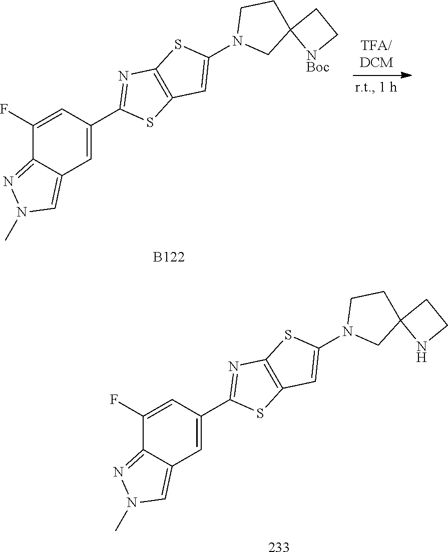 [SMILES: "B122" ; Cn1cc2cc(-c3nc4sc(N5CCC6(CCN6C(=O)OC(C)(C)C)C5)cc4s3)cc(F)c2n1 ; Cn1cc2cc(-c3nc4sc(N5CCC6(CCN6)C5)cc4s3)cc(F)c2n1 ; "233"]

A solution of tert-butyl 6-[2-(7-fluoro-2-methylindazol-5-yl)thieno[2,3-d][1,3]thiazol-5-yl]-1,6-diazaspiro[3.4]octane-1-carboxylate (30 mg, 0.06 mmol, 1.00 equiv) in DCM and TFA (3 mL/0.5 mL) was stirred for 1 h at room temperature. The resulting mixture was concentrated under reduced pressure to give a residue. The residue was purified by reverse flash chromatography (Column: YMC-Actus Triart C18, 30*150 mm, 5 μm; Mobile Phase A: Water (10 mmol/L NH₄HCO₃), Mobile Phase B: ACN; Flow rate: 60 mL/min; Gradient: 20% B to 65% B in 8 min, 65% B; Wave Length: 220 nm; RT1(min): 6.5) to afford 5-(5-{1,6-diazaspiro[3.4]octan-6-yl}thieno[2,3-d][1,3]thiazol-2-yl)-7-fluoro-2-methylindazole (5 mg, 20.84%) as a solid. LCMS (ES, m/z):400 [M+H]+. ¹H NMR (400 MHz, DMSO-d₆) δ 8.55 (d, J=2.7 Hz, 1H), 8.03 (d, J=1.3 Hz, 1H), 7.57 (dd, J=12.7, 1.4 Hz, 1H), 6.06 (s, 1H), 4.22 (s, 3H), 3.38 (s, 1H), 3.30 (s, 4H), 3.25 (s, 1H), 2.41 (m, 1H), 2.29 (m, 1H) 2.15 (m, 2H).

### Example 53: Synthesis of Compound 234

### Synthesis of Compound 234

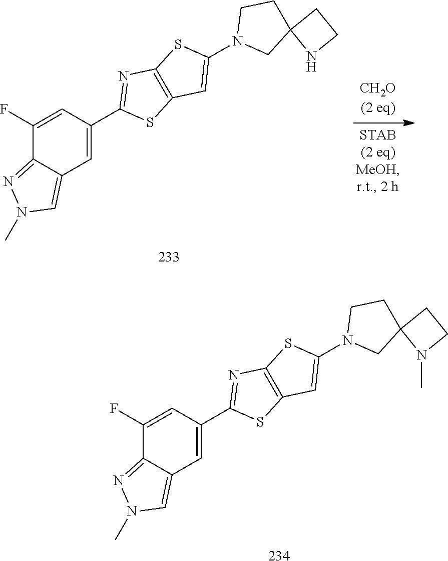 [SMILES: Cn1cc2cc(-c3nc4sc(N5CCC6(CCN6)C5)cc4s3)cc(F)c2n1 ; "233" ; CN1CCC12CCN(c1cc3sc(-c4cc(F)c5nn(C)cc5c4)nc3s1)C2 ; "234"]

A mixture of 5-(5-{1,6-diazaspiro[3.4]octan-6-yl}thieno[2,3-d][1,3]thiazol-2-yl)-7-fluoro-2-methylindazole (30 mg, 0.08 mmol, 1.00 equiv) and HCHO (4.51 mg, 0.15 mmol, 2.00 equiv) in methanol (3 mL) was stirred for 40 min at room temperature. To the reaction mixture was added STAB (31.83 mg, 0.15 mmol, 2.00 equiv). The resulting mixture was stirred for 2 h at room temperature, then extracted with ethyl acetate (3×10 mL). The combined organic layers were washed with sat. NaCl (1×10 mL), dried over anhydrous Na₂SO₄, and filtered. After filtration, the filtrate was concentrated under reduced pressure to give a resiude. The residue was purified by reverse flash chromatography (Column: YMC-Actus Triart C18, 30*150 mm, 5 m; Mobile Phase A: Water (10 mmol/L NH₄HCO₃), Mobile Phase B: ACN; Flow rate: 60 mL/min; Gradient: 40% B to 75% B in 8 min, 75% B; Wave Length: 220 nm; RT1(min): 6.05) to afford 7-fluoro-2-methyl-5-(5-{1-methyl-1,6-diazaspiro[3.4]octan-6-yl}thieno[2,3-d][1,3]thiazol-2-yl)indazole (1.50 mg, 4.83%) as a solid. LCMS (ES, m/z):414 [M+H]⁺. ¹H NMR (400 MHz, DMSO-d₆) δ 8.54 (d, J=2.8 Hz, 1H), 8.03 (s, 1H), 7.57 (d, J=12.7 Hz, 1H), 6.09 (s, 1H), 5.75 (s, 2H), 4.22 (s, 4H), δ 3.44 (s, 1H), 3.30 (s, 1H), 3.25 (d, J=10.1 Hz, 1H), 3.12 (dd, J=7.6, 5.2 Hz, 1H), 3.03 (q, J=6.8 Hz, 1H),2.25 (dt, J=12.3, 7.8 Hz, 1H), 2.15 (s, 4H), 2.07 (ddt, J=13.1, 9.8, 4.0 Hz, 3H).

### Example 54: Synthesis of Compound 236

### Synthesis of Intermediate B123

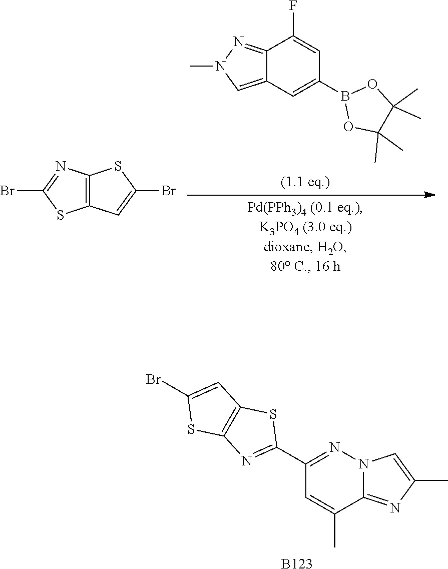

To a mixture of 2,5-dibromothieno[2,3-d] [1,3] thiazole (3.00 g, 10.03 mmol, 1.00 equiv) and 7-fluoro-2-methyl-5-(4,4,5,5-tetramethyl-1,3,2-dioxaborolan-2-yl) indazole (3.05 g, 11.04 mmol, 1.1 equiv) in dioxane (60 mL) and H₂O (12 mL) was added K₃PO₄ (6.39 g, 30.10 mmol, 3.00 equiv) and Pd(PPh₃)₄(1.16 g, 1.00 mmol, 0.10 equiv). After stirring for 16 h at 80° C. under a nitrogen atmosphere, the resulting mixture was concentrated under reduced pressure to give a residue. The residue was purified by silica gel column chromatography, eluted with PE/EA (1:1) to afford 5-{5-bromothieno[2,3-d][1,3] thiazol-2-yl}-7-fluoro-2-methylindazole (890 mg, 24.09%) as a solid. LCMS (ES, m/z):368 [M+H]⁺.

### Synthesis of Intermediate B124

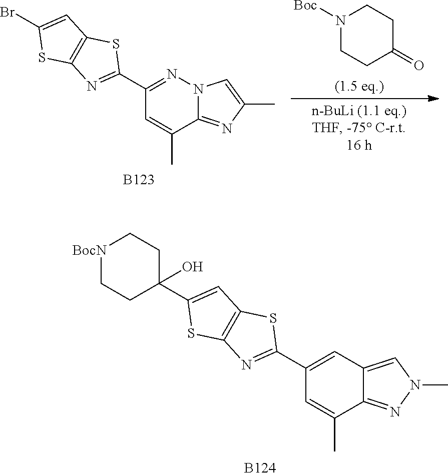 [SMILES: "B123" ; "B124" ; Cc1cn2nc(-c3nc4sc(Br)cc4s3)cc(C)c2n1 ; Cc1cc(-c2nc3sc(C4(O)CCN(C(=O)OC(C)(C)C)CC4)cc3s2)cc2cn(C)nc12 ; CC(C)(C)OC(=O)N1CCC(=O)CC1]

To a stirred solution of 5-{5-bromothieno[2,3-d] [1,3] thiazol-2-yl}-7-fluoro-2-methylindazole (100 mg, 0.27 mmol, 1.00 equiv) in tetrahydrofuran (2 mL) was added butyllithium (19.14 mg, 0.30 mmol, 1.10 equiv) portionwise at −78° C. under nitrogen atmosphere. The resulting mixture was stirred for 30 min at −78° C. under nitrogen atmosphere. To the reaction mixture was added tert-butyl 4-oxopiperidine-1-carboxylate (81.16 mg, 0.41 mmol, 1.50 equiv) in tetrahydrofuran (1 mL) dropwise at −78° C. The resulting mixture was stirred for an additional 16 h at room temperature. The reaction mixture was quenched with water at room temperature. The combined organic layers were washed with etihyl acetate (3×50 mL), dried over anhydrous Na₂SO₄, and filtered. After filtration, the filtrate was concentrated under reduced pressure to give a residue. The residue was purified by silica gel column chromatography, eluted with PE/EA (5:1) to afford tert-butyl 4-[2-(7-fluoro-2-methylindazol-5-yl) thieno[2,3-d] [1,3] thiazol-5-yl]-4-hydroxypiperidine-1-carboxylate (20 mg, 15.07%) as a solid. LCMS (ES, m/z):489 [M+H]⁺.

### Synthesis of Compound 236

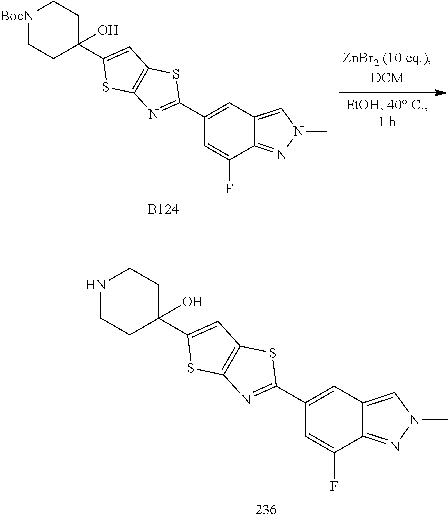 [SMILES: "B124" ; Cn1cc2cc(-c3nc4sc(C5(O)CCN(C(=O)OC(C)(C)C)CC5)cc4s3)cc(F)c2n1 ; Cn1cc2cc(-c3nc4sc(C5(O)CCNCC5)cc4s3)cc(F)c2n1]

Tert-butyl 4-[2-(7-fluoro-2-methylindazol-5-yl) thieno[2,3-d] [1,3] thiazol-5-yl]-4-hydroxypiperidine-1-carboxylate (20 mg, 0.06 mmol, 1.00 equiv), ZnBr₂ (92.19 mg, 0.41 mmol, 10.00 equiv), and DCM (1 mL) were combined at room temperature. The resulting mixture was stirred for 30 min at 40° C. under nitrogen atmosphere. Ethanol (1 mL) was added, and the reaction mixture was stirred for 10 min at 40° C. The reaction mixture was concentrated under reduced pressure to give a residue. The residue was purified by Prep-HPLC (Column, XBridge Prep OBD C18 Column, 30*150 mm, 5 μm; mobile phase, Water (10 mmol/L NH₄HCO₃) and ACN (5% ACN up to 35% in 8 min) to afford 4-[2-(7-fluoro-2-methylindazol-5-yl) thieno[2,3-d] [1,3] thiazol-5-yl] piperidin-4-ol (3.2 mg, 20.01%) as a solid. LCMS (ES, m/z):389 [M+H]⁺. ¹H NMR (400 MHz, DMSO-d₆) δ 8.62 (d, J=2.8 Hz, 1H), 8.23 (d, J=1.4 Hz, 1H), 7.65 (dd, J=12.5, 1.4 Hz, 1H), 7.34 (s, 1H), 5.60 (s, 1H), 4.24 (s, 3H), 4.04 (s, 1H), 2.92-2.86 (m, 2H), 2.75 (d, J=12.2 Hz, 2H), 1.88-1.82 (m, 2H), 1.76 (d, J=12.8 Hz, 2H).

### Example 55: Synthesis of Compound 240

### Synthesis of Intermediate B125

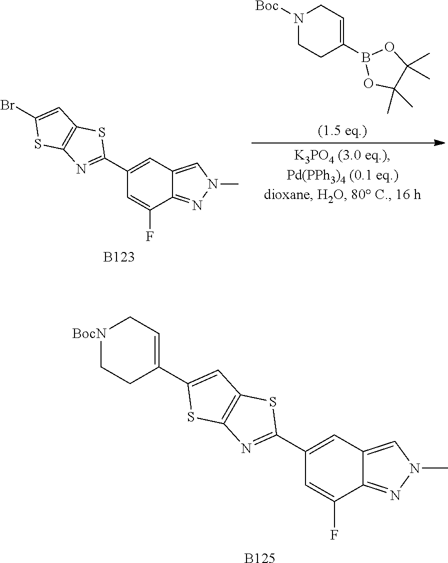 [SMILES: "B123" ; "B125" ; Cn1cc2cc(-c3nc4sc(Br)cc4s3)cc(F)c2n1 ; Cn1cc2cc(-c3nc4sc(C5=CCN(C(=O)OC(C)(C)C)CC5)cc4s3)cc(F)c2n1 ; CC(C)(C)OC(=O)N1CC=C(B2OC(C)(C)C(C)(C)O2)CC1]

To a mixture of 5-{5-bromothieno[2,3-d] [1,3] thiazol-2-yl}-7-fluoro-2-methylindazole (700 mg, 1.90 mmol, 1.00 equiv) and tert-butyl 4-(4,4,5,5-tetramethyl-1,3,2-dioxaborolan-2-yl)-3,6-dihydro-2H-pyridine-1-carboxylate (881.68 mg, 2.85 mmol, 1.50 equiv) in dioxane (15 mL) and H₂O (3 mL) was added K₃PO₄ (1210.51 mg, 5.70 mmol, 3.00 equiv) and Pd(PPh₃)₄ (219.66 mg, 0.19 mmol, 0.10 equiv). After stirring for 16 h at 80° C. under a nitrogen atmosphere, the reaction mixture was concentrated under reduced pressure to give a reside. The residue was purified by silica gel column chromatography, eluted with CH₂Cl₂/MeOH (10:1) to afford tert-butyl 4-[2-(7-fluoro-2-methylindazol-5-yl) thieno[2,3-d] [1,3] thiazol-5-yl]-3,6-dihydro-2H-pyridine-1-carboxylate (400 mg, 44.72%) as a solid. LCMS (ES, m/z):471 [M+H]⁺.

### Synthesis of Intermediate B126

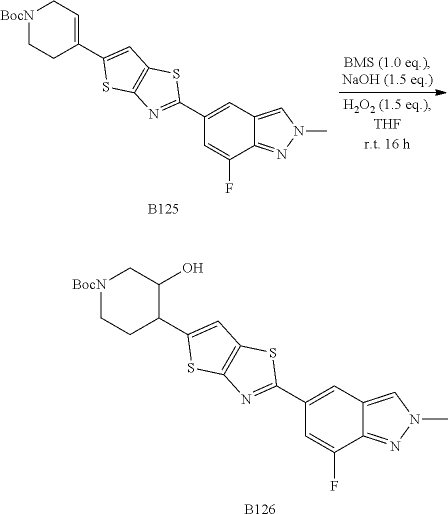 [SMILES: "B125" ; "B126" ; Cn1cc2cc(-c3nc4sc(C5=CCN(C(=O)OC(C)(C)C)CC5)cc4s3)cc(F)c2n1 ; Cn1cc2cc(-c3nc4sc(C5CCN(C(=O)OC(C)(C)C)CC5O)cc4s3)cc(F)c2n1]

To a stirred solution of tert-butyl 4-[2-(7-fluoro-2-methylindazol-5-yl) thieno[2,3-d] [1,3]thiazol-5-yl]-3,6-dihydro-2H-pyridine-1-carboxylate (400 mg, 0.85 mmol, 1.00 equiv) in THE (5 mL) was added BH₃-Me₂S (64.57 mg, 0.85 mmol, 1.00 equiv) dropwise at 0° C. under nitrogen atmosphere. The resulting mixture was stirred for 1 h at 50° C. under nitrogen atmosphere. To the reaction mixture was added NaOH (51 mg, 1.27 mmol, 1.50 equiv) and H₂O₂(43.37 mg, 1.27 mmol, 1.50 equiv) dropwise at 0° C. The resulting mixture was stirred for 16 h at room temperature under nitrogen. The aqueous layer was extracted with EtOAc (3×100 mL). The combined organic layers were combined, dried over Na₂SO₄, filtered, and concentrated under reduced pressure to yield a residue. The residue was purified by silica gel column chromatography, eluted with PE/EA (5:1) to afford tert-butyl 4-[2-(7-fluoro-2-methylindazol-5-yl) thieno[2,3-d] [1,3] thiazol-5-yl]-3-hydroxypiperidine-1-carboxylate (190 mg, 45.75%) as a solid. LCMS (ES, m/z):489 [M+H]⁺.

### Synthesis of Intermediate B127

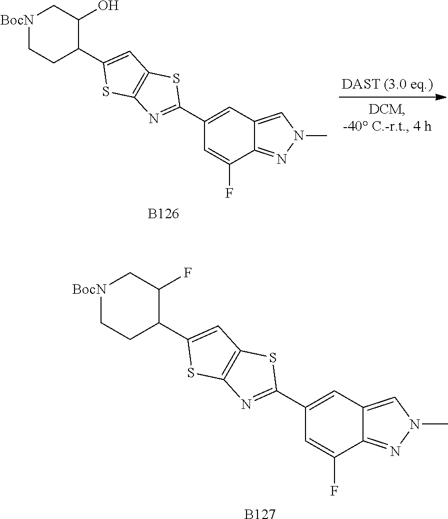 [SMILES: "B126" ; "B127" ; Cn1cc2cc(-c3nc4sc(C5CCN(C(=O)OC(C)(C)C)CC5O)cc4s3)cc(F)c2n1 ; Cn1cc2cc(-c3nc4sc(C5CCN(C(=O)OC(C)(C)C)CC5F)cc4s3)cc(F)c2n1]

To a stirred solution of tert-butyl 4-[2-(7-fluoro-2-methylindazol-5-yl) thieno[2,3-d] [1,3]thiazol-5-yl]-3-hydroxypiperidine-1-carboxylate (190 mg, 0.39 mmol, 1.00 equiv) in DCM (20 mL) was added DAST (188.04 mg, 1.16 mmol, 3.00 equiv) dropwise at −40° C. under nitrogen atmosphere. The resulting mixture was stirred for 4 h at room temperature under nitrogen atmosphere. The reaction mixture was quenched with water at 0° C., then concentrated under reduced pressure to give a residue. The residue was purified by silica gel column chromatography, eluted with PE/EA (5:1) to afford tert-butyl 3-fluoro-4-[2-(7-fluoro-2-methylindazol-5-yl) thieno[2,3-d] [1,3] thiazol-5-yl] piperidine-1-carboxylate (100 mg, 52.42%) as a solid. LCMS (ES, m/z):491 [M+H]⁺.

### Synthesis of Compound 240

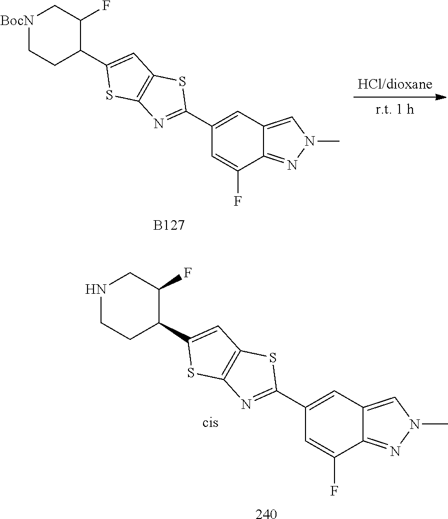 [SMILES: "B127" ; Cn1cc2cc(-c3nc4sc(C5CCN(C(=O)OC(C)(C)C)CC5F)cc4s3)cc(F)c2n1 ; Cn1cc2cc(-c3nc4sc([C@H]5CCNC[C@H]5F)cc4s3)cc(F)c2n1 ; "240"]

Tert-butyl 3-fluoro-4-[2-(7-fluoro-2-methylindazol-5-yl) thieno[2,3-d] [1,3] thiazol-5-yl]piperidine-1-carboxylate (90 mg, 0.18 mmol, 1.00 equiv) and HCl (gas) in 1,4-dioxane (1 mL) were combined at room temperature. The resulting mixture was stirred for 1 h at room temperature under nitrogen atmosphere, then concentrated under reduced pressure to give a residue. The residue was purified by Prep-HPLC (2 SHIMADZU (HPLC-01): Column, XBridge Prep OBD C18 Column, 30*150 mm, 5 μm; mobile phase, Water (10 mmol/L NH₄HCO₃) and ACN (20% ACN up to 45% in 8 min) to afford 7-fluoro-5-{5-[(3S,4S)-3-fluoropiperidin-4-yl] thieno[2,3-d] [1,3] thiazol-2-yl}-2-methylindazole (8.5 mg, 11.82%) as a solid. LCMS (ES, m/z):391 [M+H]⁺. ¹H NMR (400 MHz, DMSO-d₆) δ 8.68 (d, J=2.7 Hz, 1H), 8.29 (d, J=1.4 Hz, 1H), 7.71 (dd, J=12.5, 1.4 Hz, 1H), 7.46 (s, 1H), 4.76 (dtd, J=49.5, 10.1, 4.8 Hz, 1H), 4.31 (s, 3H), 3.28 (t, J=5.7 Hz, 1H), 3.14 (ddt, J=16.8, 9.6, 4.2 Hz, 2H), 2.69 (q, J=13.0, 12.4 Hz, 2H), 2.17 (ddd, J=13.3, 8.6, 4.6 Hz, 1H), 1.71-1.58 (m, 1H).

### Example 56: Synthesis of Compound 241

### Synthesis of Intermediate B128

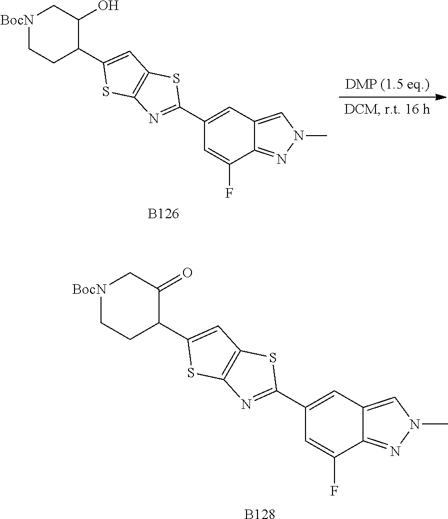 [SMILES: "B126" ; "B128" ; Cn1cc2cc(-c3nc4sc(C5CCN(C(=O)OC(C)(C)C)CC5O)cc4s3)cc(F)c2n1 ; Cn1cc2cc(-c3nc4sc(C5CCN(C(=O)OC(C)(C)C)CC5=O)cc4s3)cc(F)c2n1]

Tert-butyl 4-[2-(7-fluoro-2-methylindazol-5-yl)thieno[2,3-d][1,3] thiazol-5-yl]-3-hydroxypiperidine-1-carboxylate (180 mg, 0.36 mmol, 1.00 equiv), DCM (3 mL), and DMP (234.38 mg, 0.55 mmol, 1.50 equiv) were combined at 0° C. The resulting mixture was stirred for 16 h at room temperature under nitrogen atmosphere. The reaction mixture was concentrated under reduced pressure to give a residue. The residue was used in the next step directly without further purification. LCMS (ES, m/z):487 [M+H]⁺.

### Synthesis of Intermediate B129

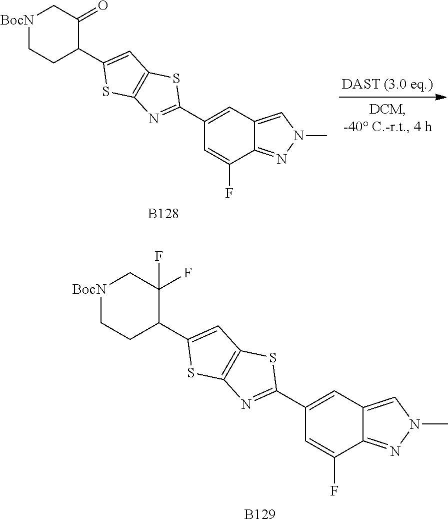 [SMILES: "B128" ; "B129" ; Cn1cc2cc(-c3nc4sc(C5CCN(C(=O)OC(C)(C)C)CC5=O)cc4s3)cc(F)c2n1 ; Cn1cc2cc(-c3nc4sc(C5CCN(C(=O)OC(C)(C)C)CC5(F)F)cc4s3)cc(F)c2n1]

Tert-butyl 4-[2-(7-fluoro-2-methylindazol-5-yl)thieno[2,3-d][1,3] thiazol-5-yl]-3-oxopiperidine-1-carboxylate (190 mg, 0.39 mmol, 1.00 equiv), DCM (3 mL), and DAST (94.41 mg, 0.58 mmol, 1.50 equiv) were combined at 0° C. The resulting mixture was stirred for 4 h at room temperature under nitrogen atmosphere, then concentrated under reduced pressure to give a residue. The residue was purified by silica gel column chromatography, eluted with PE/EA (1:1) to afford tert-butyl 3,3-difluoro-4-[2-(7-fluoro-2-methylindazol-5-yl) thieno[2,3-d] [1,3] thiazol-5-yl] piperidine-1-carboxylate (60 mg, 30.21%) as a solid. LCMS (ES, m/z):509 [M+H]⁺.

### Synthesis of Compound 241

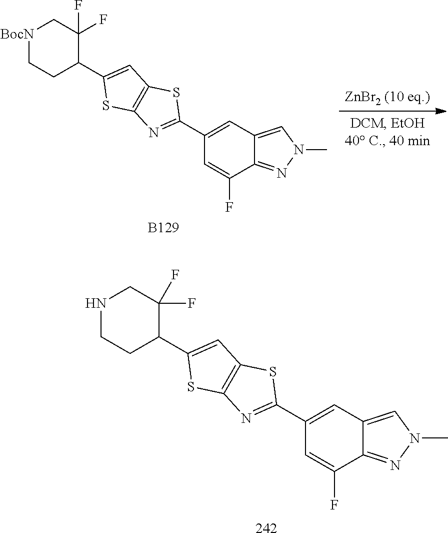 [SMILES: "B129" ; Cn1cc2cc(-c3nc4sc(C5CCN(C(=O)OC(C)(C)C)CC5(F)F)cc4s3)cc(F)c2n1 ; Cn1cc2cc(-c3nc4sc(C5CCNCC5(F)F)cc4s3)cc(F)c2n1 ; "242"]

A mixture of tert-butyl 3,3-difluoro-4-[2-(7-fluoro-2-methylindazol-5-yl)thieno[2,3-d][1,3]thiazol-5-yl]piperidine-1-carboxylate (56 mg, 0.11 mmol, 1.00 equiv) and ZnBr₂ (248 mg, 1.10 mmol, 10.00 equiv) in DCM (0.50 mL) was stirred for 30 min at 40° C. under nitrogen atmosphere. To the reaction mixture was added ethanol (0.50 mL), and the resulting mixture was stirred for an additional 10 min. The reaction mixture was concentrated under reduced pressure to give a residue. The residue was purified by Prep-HPLC (2 SHIMADZU (HPLC-01): Column, YMC-Actus Triart C18, 30*150 mm, 5 μm; mobile phase, Water (10 mmol/L NH₄HCO₃) and ACN (25% ACN up to 70% in 8 min) to afford 5-[5-(3,3-difluoropiperidin-4-yl) thieno[2,3-d] [1,3] thiazol-2-yl]-7-fluoro-2-methylindazole (1.1 mg, 2.43%) as a solid. LCMS (ES, m/z):409 [M+H]⁺. ¹H NMR (400 MHz, DMSO-d₆) δ 8.62 (d, J=2.8 Hz, 1H), 8.24 (d, J=1.3 Hz, 1H), 7.65 (dd, J=12.5, 1.4 Hz, 1H), 7.42 (s, 1H), 4.24 (s, 3H), 3.70 (d, J=14.9 Hz, 1H), 3.15 (s, 1H), 2.99 (d, J=12.9 Hz, 1H), 2.90 (d, J=13.4 Hz, 1H), 2.82 (d, J=13.5 Hz, 1H), 2.01 (s, 1H), 1.89 (d, J=12.6 Hz, 1H).

### Example 57: Synthesis of Compound 237

### Synthesis of Intermediate B130

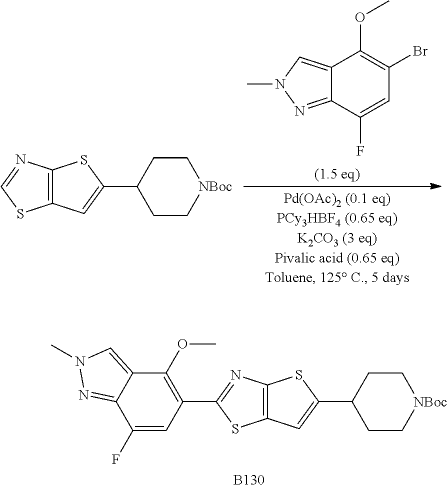 [SMILES: COc1c(Br)cc(F)c2nn(C)cc12 ; "B130" ; CC(C)(C)OC(=O)N1CCC(c2cc3scnc3s2)CC1 ; COc1c(-c2nc3sc(C4CCN(C(=O)OC(C)(C)C)CC4)cc3s2)cc(F)c2nn(C)cc12]

To a stirred mixture of tert-butyl 4-{thieno[2,3-d][1,3]thiazol-5-yl}piperidine-1-carboxylate (100 mg, 0.31 mmol, 1.00 equiv), 5-bromo-7-fluoro-4-methoxy-2-methylindazole (119.77 mg, 0.46 mmol, 1.50 equiv), Pd(OAc)₂ (6.92 mg, 0.03 mmol, 0.10 equiv), and PCy₃HBF₄ (73.77 mg, 0.20 mmol, 0.65 equiv) in toluene (3 ml) was added pivalic acid (20.46 mg, 0.20 mmol, 0.65 equiv) and K₂CO₃ (127.79 mg, 0.92 mmol, 3.00 equiv). The reaction mixture was stirred for 5 days at 125° C. under nitrogen atmosphere, then extracted with ethyl acetate (3×10 mL). The combined organic layers were washed with sat. NaCl (1×10 mL), dried over anhydrous Na₂SO₄, and filtered. After filtration, the filtrate was concentrated under reduced pressure to give a residue. The residue was purified by silica gel column chromatography, eluted with PE/EA (1:1) to afford tert-butyl 4-[2-(7-fluoro-4-methoxy-2-methylindazol-5-yl)thieno[2,3-d][1,3]thiazol-5-yl]piperidine-1-carboxylate (60 mg, 38.73%) as a solid. LCMS (ES, m/z):503 [M+H]⁺.

### Synthesis of Compound 237

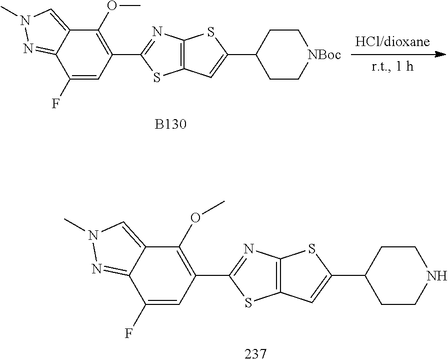 [SMILES: "B130" ; COc1c(-c2nc3sc(C4CCN(C(=O)OC(C)(C)C)CC4)cc3s2)cc(F)c2nn(C)cc12 ; COc1c(-c2nc3sc(C4CCNCC4)cc3s2)cc(F)c2nn(C)cc12 ; "237"]

A mixture of tert-butyl 4-[2-(7-fluoro-4-methoxy-2-methylindazol-5-yl)thieno[2,3-d][1,3]thiazol-5-yl] piperidine-1-carboxylate (60 mg, 0.12 mmol, 1.00 equiv) and HCl (gas) in 1,4-dioxane (5 mL) was stirred for 1 h at room temperature. The resulting mixture was concentrated under reduced pressure to give a residue. The residue was purified by reverse flash chromatography (Column: YMC-Actus Triart C18, 30*150 mm, 5 m; Mobile Phase A: Water (10 mmol/L NH₄HCO₃), Mobile Phase B: ACN; Flow rate: 60 mL/min; Gradient: 15% B to 70% B in 8 min, 70% B; Wave Length: 220 nm; RT1(min): 6.18) to afford 7-fluoro-4-methoxy-2-methyl-5-[5-(piperidin-4-yl)thieno[2,3-d][1,3]thiazol-2-yl]indazole (10.10 mg, 21.02%) as a solid. LCMS (ES, m/z): 403 [M+H]+. ¹H NMR (400 MHz, DMSO-d₆) δ 9.00 (d, J=2.7 Hz, 1H), 7.85 (d, J=12.5 Hz, 1H), 7.23 (d, J=1.0 Hz, 1H), 4.28 (s, 3H), 4.22 (s, 3H), 2.99 (ddt, J=24.0, 11.6, 3.5 Hz, 3H), 2.60 (td, J=12.1, 2.4 Hz, 2H), 1.98-1.89 (m, 2H), 1.54 (qd, J=12.2, 3.9 Hz, 2H).

### Example 58: Synthesis of Compound 238

### Synthesis of Intermediate B131

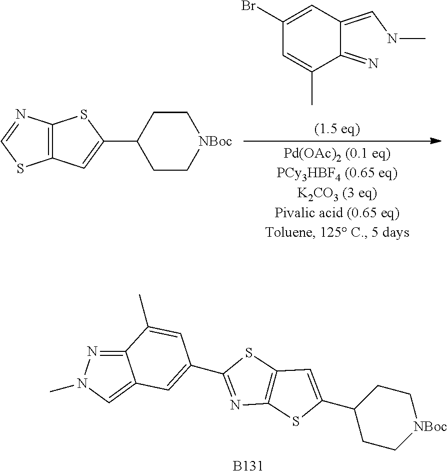 [SMILES: CN/C=C1/C=C(Br)C=C(C)C1=N ; CC(C)(C)OC(=O)N1CCC(c2cc3scnc3s2)CC1 ; "B131" ; Cc1cc(-c2nc3sc(C4CCN(C(=O)OC(C)(C)C)CC4)cc3s2)cc2cn(C)nc12]

To a stirred mixture of tert-butyl 4-{thieno[2,3-d][1,3]thiazol-5-yl}piperidine-1-carboxylate (100 mg, 0.31 mmol, 1.00 equiv), 5-bromo-2,7-dimethylindazole (104.06 mg, 0.46 mmol, 1.50 equiv), Pd(OAc)₂ (6.92 mg, 0.03 mmol, 0.10 equiv), and PCy₃HBF₄ (56.18 mg, 0.20 mmol, 0.65 equiv) in toluene (3 ml) was added pivalic acid (20.46 mg, 0.200 mmol, 0.65 equiv) and K₂CO₃ (127.79 mg, 0.92 mmol, 3.00 equiv). The reaction mixture was stirred for 5 days at 125° C. under nitrogen atmosphere, then extracted with ethyl acetate (3×10 mL). The combined organic layers were washed with sat. NaCl (1×10 mL), dried over anhydrous Na₂SO₄, and filtered. After filtration, the filtrate was concentrated under reduced pressure to give a residue. The residue was purified by silica gel column chromatography, eluted with PE/EA (1:1) to afford tert-butyl 4-[2-(2,7-dimethylindazol-5-yl)thieno [2,3-d][1,3]thiazol-5-yl]piperidine-1-carboxylate (50 mg, 34.62%) as a solid. LCMS (ES, m/z):469 [M+H]⁺.

### Synthesis of Compound 238

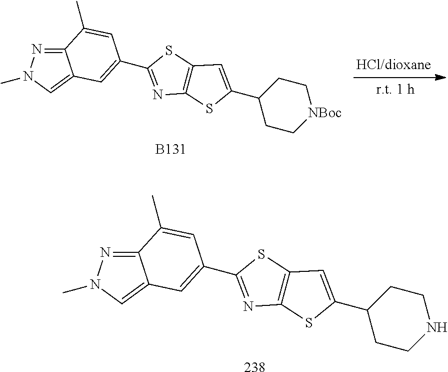 [SMILES: "B131" ; Cc1cc(-c2nc3sc(C4CCN(C(=O)OC(C)(C)C)CC4)cc3s2)cc2cn(C)nc12 ; Cc1cc(-c2nc3sc(C4CCNCC4)cc3s2)cc2cn(C)nc12 ; "238"]

A solution of tert-butyl 4-[2-(2,7-dimethylindazol-5-yl)thieno[2,3-d][1,3]thiazol-5-yl]piperidine-1-carboxylate (50 mg, 0.11 mmol, 1.00 equiv) and HCl (gas) in 1,4-dioxane (5 mL) was stirred for 1 h at room temperature. The resulting mixture was concentrated under reduced pressure to give a residue. The residue was purified by reverse flash chromatography (Column: XBridge Prep OBD C18 Column, 30*150 mm, 5 m; Mobile Phase A: Water (10 mmol/L NH₄HCO₃), Mobile Phase B: ACN; Flow rate: 60 mL/min; Gradient: 10% B to 50% B in 8 min, 50% B; Wave Length: 220 nm; RT1(min): 6.42) to afford 2,7-dimethyl-5-[5-(piperidin-4-yl)thieno[2,3-d][1,3]thiazol-2-yl]indazole (16.40 mg, 41.71%) as a solid. LCMS (ES, m/z):369 [M+H]⁺. ¹H NMR (400 MHz, DMSO-d₆) δ 8.45 (s, 1H), 8.18 (d, J=1.7 Hz, 1H), 7.65 (t, J=1.4 Hz, 1H), 7.25 (d, J=1.0 Hz, 1H), 4.20 (s, 3H), 3.08-2.94 (m, 3H), 2.62 (td, J=12.2, 2.4 Hz, 2H), 2.57 (s, 3H), 1.99-1.91 (m, 2H), 1.55 (qd, J=12.2, 3.9 Hz, 2H).

### Example 59: Synthesis of Compound 242

### Synthesis of Compound 242

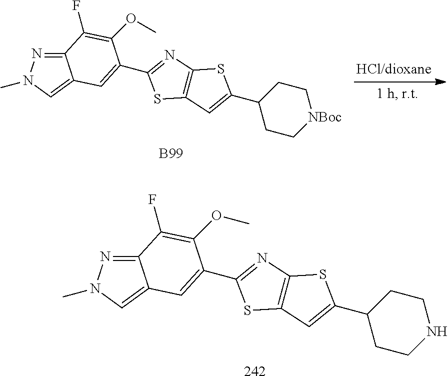 [SMILES: "B99" ; COc1c(-c2nc3sc(C4CCN(C(=O)OC(C)(C)C)CC4)cc3s2)cc2cn(C)nc2c1F ; COc1c(-c2nc3sc(C4CCNCC4)cc3s2)cc2cn(C)nc2c1F ; "242"]

A mixture of tert-butyl 4-[2-(7-fluoro-6-methoxy-2-methylindazol-5-yl)thieno[2,3-d][1,3]thiazol-5-yl] piperidine-1-carboxylate (50 mg, 0.10 mmol, 1.00 equiv) and HCl (gas) in 1,4-dioxane (5 mL) was stirred for 1 h at room temperature. The resulting mixture was concentrated under reduced pressure to give a reside. The residue was purified by reverse flash chromatography (Column: YMC-Actus Triart C18, 30*150 mm, 5 m; Mobile Phase A: Water (10 mmol/L NH₄HCO₃), Mobile Phase B: ACN; Flow rate: 60 mL/min; Gradient: 15% B to 70% B in 8 min, 70% B; Wave Length: 220 nm; RT1(min): 6.02) to afford 7-fluoro-6-methoxy-2-methyl-5-[5-(piperidin-4-yl)thieno[2,3-d][1,3]thiazol-2-yl]indazole (18.70 mg, 46.70%) as a solid. LCMS (ES, m/z):403 [M+H]⁺. ¹H NMR (400 MHz, DMSO-d₆) δ 8.60 (d, J=2.7 Hz, 1H), 8.50 (d, J=0.9 Hz, 1H), 7.26 (d, J=1.0 Hz, 1H), 4.20 (s, 3H), 4.06 (d, J=1.5 Hz, 3H), 3.08-2.94 (m, 3H), 2.61 (td, J=12.2, 2.5 Hz, 2H), 1.99-1.90 (m, 2H), 1.59 (dd, J=12.1, 3.9 Hz, 2H).

### Example 60: Synthesis of Compounds 208, 210, 211, 214, 216, 218, 219, 221-226, and 228

### Synthesis of Intermediate B132

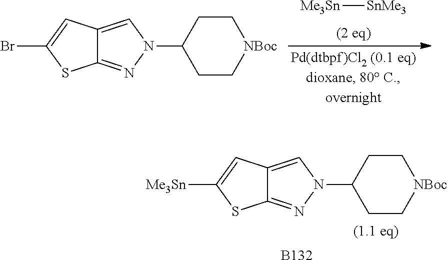

A mixture of tert-butyl 4-{5-bromothieno[2,3-c]pyrazol-2-yl}piperidine-1-carboxylate (500 mg, 1.294 mmol, 1.00 equiv), hexamethyldistannane (848.10 mg, 2.588 mmol, 2 equiv), and Pd(DtBPF)Cl₂ (84.36 mg, 0.129 mmol, 0.1 equiv) in 1,4-dioxane (10 mL, 113.471 mmol, 87.69 equiv) was stirred overnight at 80° C. under nitrogen atmosphere. The reaction mixture was allowed to cool to room temperature, then quenched with sat. KF (aq.) (30 mL) at 0° C. The resulting mixture was extracted with ethyl acetate (3×30 mL). The combined organic layers were washed with brine (2×20 mL), dried over anhydrous Na₂SO₄, and filtered. After filtration, the filtrate was concentrated under reduced pressure to give a residue. The residue (tert-butyl 4-[5-(trimethylstannyl)thieno[2,3-c]pyrazol-2-yl]piperidine-1-carboxylate (950 mg, 78.05%)) was used in the next step directly without further purification.

### Synthesis of Intermediate B133

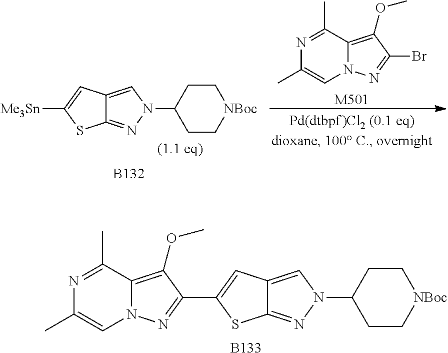

A mixture of 2-bromo-3-methoxy-4,6-dimethylpyrazolo[1,5-a]pyrazine (100 mg, 0.390 mmol, 1.00 equiv), tert-butyl 4-[5-(trimethylstannyl)thieno[2,3-c]pyrazol-2-yl]piperidine-1-carboxylate (201.97 mg, 0.429 mmol, 1.1 equiv), and Pd(DtBPF)Cl₂ (25.45 mg, 0.039 mmol, 0.1 equiv) in 1,4-dioxane (5 mL, 56.750 mmol, 145.34 equiv) was stirred overnight at 100° C. under nitrogen atmosphere. The resulting mixture was concentrated under reduced pressure to give a residue. The residue was purified by silica gel column chromatography, eluted with DCM/EA (2:1) to afford tert-butyl 4-(5-{3-methoxy-4,6-dimethylpyrazolo[1,5-a]pyrazin-2-yl}thieno [2,3-c]pyrazol-2-yl)piperidine-1-carboxylate (125 mg, 66.33%) as a solid.

### Synthesis of Compound 219

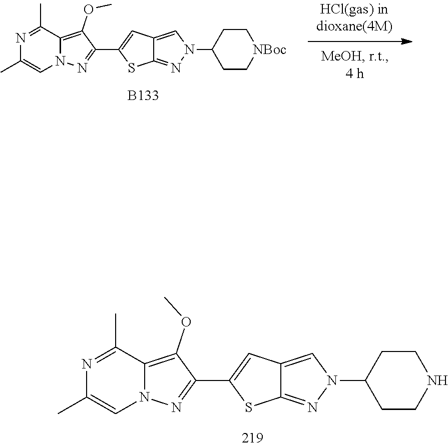 [SMILES: "B133" ; COc1c(-c2cc3cn(C4CCN(C(=O)OC(C)(C)C)CC4)nc3s2)nn2cc(C)nc(C)c12 ; COc1c(-c2cc3cn(C4CCNCC4)nc3s2)nn2cc(C)nc(C)c12]

To a stirred solution of tert-butyl 4-(5-{3-methoxy-4,6-dimethylpyrazolo[1,5-a]pyrazin-2-yl}thieno[2,3-c]pyrazol-2-yl)piperidine-1-carboxylate (50 mg, 0.104 mmol, 1.00 equiv) in methanol (1.25 mL) was added HCl (gas) in 1,4-dioxane (1.25 mL) dropwise at room temperature under air atmosphere. The resulting mixture was stirred for 4 h at room temperature under air atmosphere. The resulting mixture was concentrated under vacuum to give a residue. The residue was purified by Prep-HPLC (Column: XBridge Prep OBD C18 Column, 30×150 mm, 5 m; Mobile Phase A: Water (10 mmol/L NH₄HCO₃), Mobile Phase B: ACN; Flow rate: 60 mL/min; Gradient: 5% B to 40% B in 8 min; Wave Length: 220 nm; RT1(min): 7.30) to afford 4-(5-{3-methoxy-4,6-dimethylpyrazolo[1,5-a]pyrazin-2-yl}thieno[2,3-c]pyrazol-2-yl) piperidine (17.0 mg, 42.51%) as a solid.

Compounds 208, 210, 211, 214, 216, 218, 219, 221-226, and 228 were prepared according to the procedures described herein and outlined in this Example 60. The table below provides intermediates used in these procedures and final compound characterization data.

| | | LCMS | |
|---|---|---|---|
| | | (ESI, m/z) | |
| Compound No. and Structure | Coupling Reagent | [M + H]⁺ | ¹H NMR δ |
| 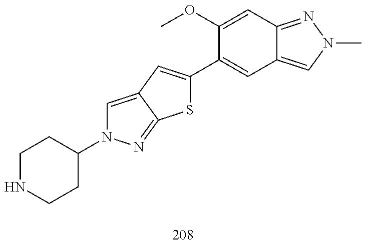 [SMILES: COc1cc2nn(C)cc2cc1-c1cc2cn(C3CCNCC3)nc2s1] | 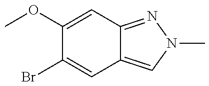 | 368 | (400 MHz, DMSO- d₆): δ 8.28 (s, 1H), 8.05 (s, 1H), 7.86 (s, 1H), 7.34 (s, 1H), 7.07 (s, 1H), 4.35 (tt, J = 11.5, 4.1 Hz, 1H), 4.12 (s, 3H), 3.90 (s, 3H), 3.06 (dt, J = 12.4, 3.3 Hz, 2H), 2.61 (td, J = 12.3, 2.5 Hz, 2H), 2.30 (s, 1H), 2.08- 1.95 (m, 2H), 1.87 (qd, J = 12.0, 4.1 Hz, 2H) |
| 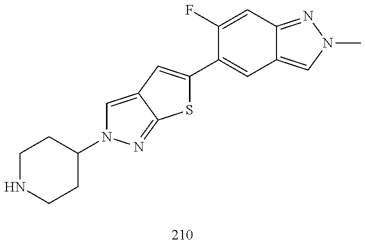 [SMILES: Cn1cc2cc(-c3cc4cn(C5CCNCC5)nc4s3)c(F)cc2n1] | 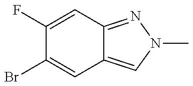 | 356 | (400 MHz, DMSO- d₆): δ 8.45 (s, 1H), 8.12 (s, 1H), 8.02 (d, J = 7.7 Hz, 1H), 7.49 (d, J = 12.7 Hz, 1H), 7.33 (d, J = 2.0 Hz, 1H), 4.37 (tt, J = 11.5, 4.1 Hz, 1H), 4.17 (s, 3H), 3.06 (dt, J = 12.5, 3.5 Hz, 2H), 2.61 (td, J = 12.3, 2.5 Hz, 2H), 2.28 (s, 1H), 2.01 (ddt, J = 11.4, 4.5, 2.2 Hz, 2H), 1.88 |
| | | | (qd, J = 12.0, 4.1 Hz, |
| | | | 2H) |
| 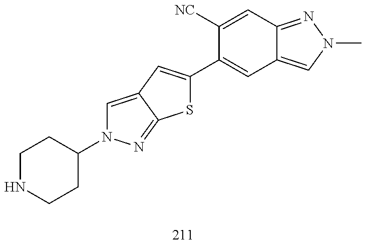 [SMILES: Cn1cc2cc(-c3cc4cn(C5CCNCC5)nc4s3)c(C#N)cc2n1] | 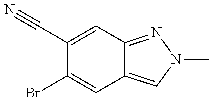 | 363 | (400 MHz, DMSO- d₆): δ 8.59 (s, 1H), 8.46 (s, 1H), 8.21 (s, 1H), 8.04 (s, 1H), 7.35 (s, 1H), 4.39 (ddt, J = 11.3, 8.1, 4.1 Hz, 1H), 4.28 (s, 3H), 3.07 (d, J = 12.7 Hz, 2H), 2.94 (s, 1H), 2.68-2.58 (m, 2H), 2.11-1.97 (m, 2H), 1.90 (qd, J = 11.9, 4.0 Hz, 2H) |
| 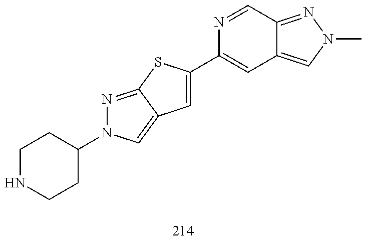 [SMILES: Cn1cc2cc(-c3cc4cn(C5CCNCC5)nc4s3)ncc2n1] | 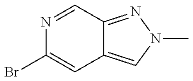 | 339 | (400 MHz, 353K, DMSO-d₆): δ 9.18 (s, 1H), 9.14 (t, J = 1.2 Hz, 1H), 8.97 (s, 1H), 8.47 (s, 1H), 8.12 (d, J = 1.4 Hz, 1H), 8.05 (s, 1H), 7.59 (s, 1H), 4.68 (p, J = 8.1, 7.5 Hz, 1H), 4.27 (s, 3H), 3.42 (d, J = 13.0 Hz, 2H), 3.11 (s, 2H), 2.31 (td, J = 8.8, 7.5, 4.2 Hz, 4H) |
| 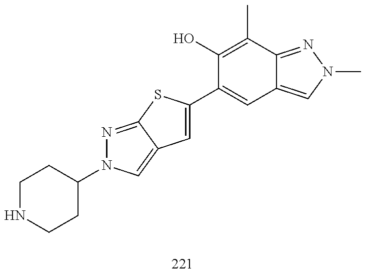 [SMILES: Cc1c(O)c(-c2cc3cn(C4CCNCC4)nc3s2)cc2cn(C)nc12] | 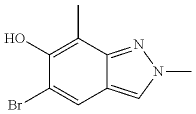 | 368 | (400 MHz, 353K, DMSO-d₆): δ 12.95 (s, 1H), 8.03 (s, 1H), 7.99 (s, 1H), 7.79 (s, 1H), 7.28 (s, 1H), 4.35 (tt, J = 11.5, 4.1 Hz, 1H), 3.65 (s, 3H), 3.13-3.07 (m, 2H), 2.64 (td, J = 12.2, 2.6 Hz, 2H), 2.48 (s, 3H), 2.10-2.00 (m, 2H), 1.90 (qd, J = 11.9, 4.2 Hz, 3H) |
| 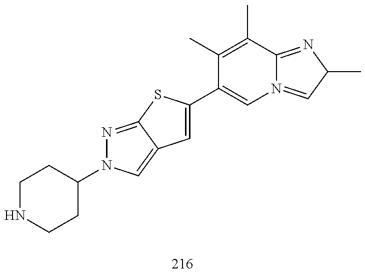 | 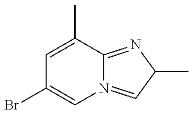 | 352 | (400 MHz, 353K, DMSO-d₆): δ 8.55 (d, J = 1.8 Hz, 1H), 8.03 (s, 1H), 7.67 (d, J = 1.0 Hz, 1H), 7.32 (d, J = 7.5 Hz, 2H), 4.35 (tt, J = 11.4, 4.1 Hz, 1H), 3.09 (dd, J = 7.8, 4.5 Hz, 2H), 2.64 (td, J = 12.2, 2.6 Hz, 2H), 2.51 (s, 3H), 2.35 (d, J = 0.8 Hz, 3H), 2.04 (ddt, J = 11.5, 4.4, 2.3 Hz, 2H), 1.89 (qd, J = 11.9, 4.2 Hz, 3H) |
| 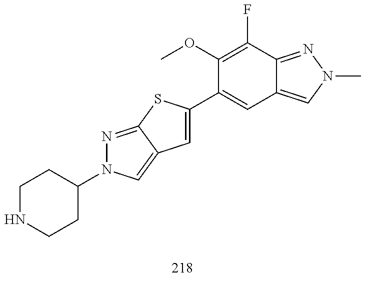 [SMILES: COc1c(-c2cc3cn(C4CCNCC4)nc3s2)cc2cn(C)nc2c1F] | 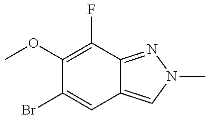 | 386 | (400 MHz, 353K, DMSO-d₆): δ 8.39 (d, J = 2.9 Hz, 1H), 8.02 (s, 1H), 7.73 (d, J = 1.0 Hz, 1H), 7.33 (s, 1H), 4.36 (tt, J = 11.4, 4.1 Hz, 1H), 4.18 (s, 3H), 3.89 (d, J = 1.3 Hz, 3H), 3.13- 3.06 (m, 2H), 2.64 (td, J = 12.3, 2.6 Hz, 2H), 2.09-2.00 (m, 2H), 1.89 (qd, J = 11.9, 4.2 Hz, 3H) |
| 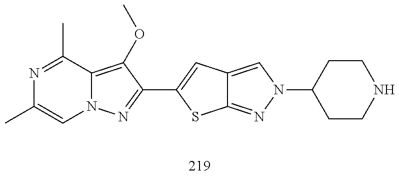 [SMILES: COc1c(-c2cc3cn(C4CCNCC4)nc3s2)nn2cc(C)nc(C)c12] | 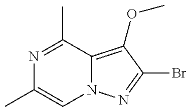 | 383 | (400 MHz, 353K, DMSO-d₆): δ 8.23 (s, 1H), 8.08 (s, 1H), 7.53 (s, 1H), 4.37 (tt, J = 11.4, 4.1 Hz, 1H), 3.92 (s, 3H), 3.14-3.07 (m, 2H), 2.76 (s, 3H), 2.64 (td, J = 12.3, 2.6 Hz, 2H), 2.38 (d, J = 1.1 Hz, 3H), 2.10-1.98 |
| | | | (m, 2H), 1.90 (qd, J = |
| | | | 11.8, 4.2 Hz, 3H) |
| 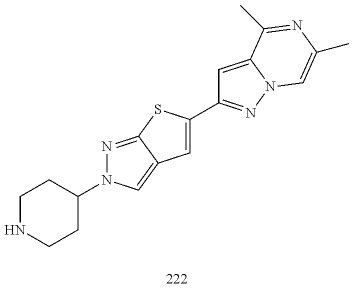 [SMILES: Cc1cn2nc(-c3cc4cn(C5CCNCC5)nc4s3)cc2c(C)n1] | 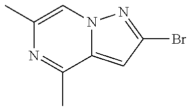 | 353 | (400 MHz, DMSO- d₆): δ 8.47 (s, 1H), 8.17 (s, 1H), 7.61 (s, 1H), 7.37 (s, 1H), 4.36 (t, J = 10.3 Hz, 1H), 3.07 (d, J = 12.2 Hz, 2H), 2.68 (s, 3H), 2.61 (t, J = 11.9 Hz, 2H), 2.41 (s, 3H), 2.01 (d, J = 11.9 Hz, 2H), 1.88 (qd, J = 11.9, 4.0 Hz, 2H) |
| 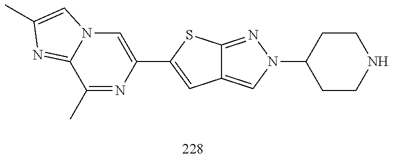 [SMILES: Cc1cn2cc(-c3cc4cn(C5CCNCC5)nc4s3)nc(C)c2n1] | 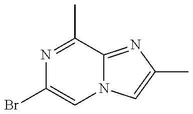 | 353 | (400 MHz, DMSO- d₆): δ 8.95 (s, 1H), 8.11 (s, 1H), 7.83 (s, 1H), 7.54 (s, 1H), 4.36 (tt, J = 11.7, 4.1 Hz, 1H), 3.15-2.99 (m, 2H), 2.73 (s, 3H), 2.62 (td, J = 12.4, 2.5 Hz, 2H), 2.41 (s, 3H), 2.12-1.96 (m, 2H), |
| | | | 1.89 (qd, J = 12.0, |
| | | | 4.1 Hz, 2H) |
| 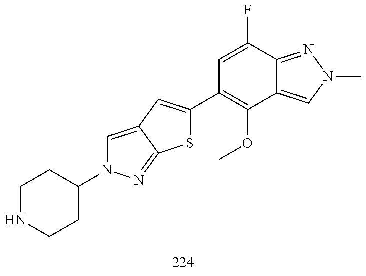 [SMILES: COc1c(-c2cc3cn(C4CCNCC4)nc3s2)cc(F)c2nn(C)cc12] | 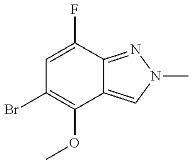 | 386 | (400 MHz, DMSO- d₆): δ 8.78 (d, J = 2.7 Hz, 1H), 8.06 (s, 1H), 7.48 (d, J = 12.8 Hz, 1H), 7.45 (s, 1H), 4.35 (tt, J = 11.5, 4.1 Hz, 1H), 4.19 (s, 3H), 4.03 (s, 3H), 3.12- 3.00 (m, 2H), 2.61 (td, J = 12.4, 2.6 Hz, 2H), 2.07-1.95 (m, 2H), 1.88 (qd, J = 12.0, 4.1 Hz, 2H) |
| 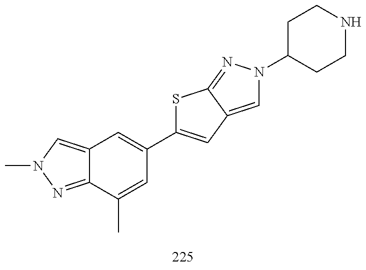 [SMILES: Cc1cc(-c2cc3cn(C4CCNCC4)nc3s2)cc2cn(C)nc12] | 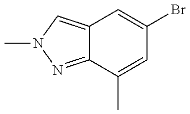 | 352 | (400 MHz, DMSO- d₆): δ 8.32 (s, 1H), 8.06 (s, 1H), 7.80- 7.65 (m, 1H), 7.39 (d, J = 21.2 Hz, 2H), 4.35 (tt, J = 11.4, 4.1 Hz, 1H), 4.17 (s, 3H), 3.15-3.00 (m, 2H), 2.67-2.58 (m, 2H), 2.54 (s, 3H), 2.09- 1.95 (m, 2H), 1.95- 1.78 (m, 2H) |
| 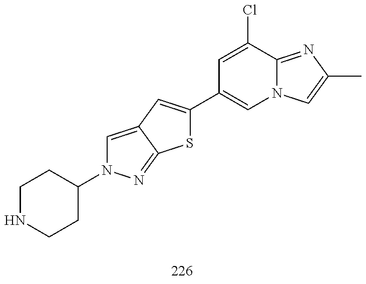 [SMILES: Cc1cn2cc(-c3cc4cn(C5CCNCC5)nc4s3)cc(Cl)c2n1] | 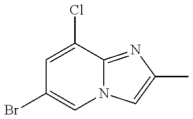 | 372 | (400 MHz, DMSO- d₆): δ 8.77 (d, J = 1.7 Hz, 1H), 8.15 (s, 1H), 7.84 (t, J = 1.7 Hz, 2H), 7.55 (s, 1H), 4.36 (tt, J = 11.5, 3.7 Hz, 1H), 3.06 (dt, J = 12.6, 3.4 Hz, 2H), 2.61 (td, J = 12.3, 2.5 Hz, 2H), 2.36 (s, 3H), 2.07-1.95 (m, 2H), 1.87 (qd, J = 12.0, 4.1 Hz, 2H) |
| 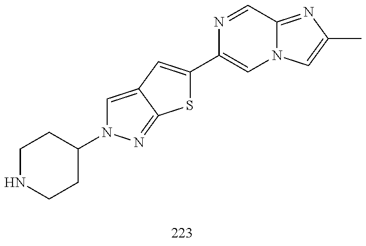 [SMILES: Cc1cn2cc(-c3cc4cn(C5CCNCC5)nc4s3)ncc2n1] | 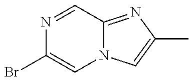 | 339 | (400 MHz, DMSO- d₆): δ 9.12 (s, 1H), 8.94 (s, 1H), 8.14 (s, 1H), 7.90 (s, 1H), 7.58 (d, J = 1.9 Hz, 1H), 4.37 (d, J = 12.1 Hz, 1H), 3.06 (d, J = 12.3 Hz, 2H), 2.61 (t, J = 12.3 Hz, 2H), 2.43 (s, 3H), 2.01 (d, J = 12.3 Hz, 2H), 1.87 (ddt, J = 15.8, 11.9, 6.5 Hz, 2H) |

### Example 61: Synthesis of Compounds 213 and 215

### Synthesis of Intermediate B134

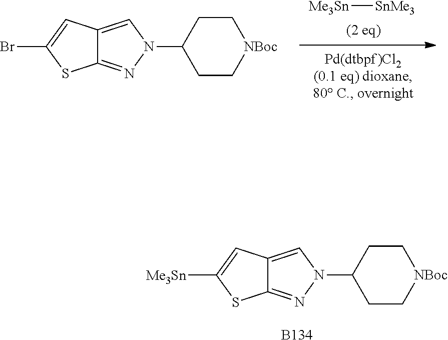

A mixture of tert-butyl 4-{5-bromothieno[2,3-c]pyrazol-2-yl}piperidine-1-carboxylate (500 mg, 1.294 mmol, 1.00 equiv), hexamethyldistannane (848.10 mg, 2.588 mmol, 2 equiv), and Pd(DtBPF)Cl₂ (84.36 mg, 0.129 mmol, 0.1 equiv) in 1,4-dioxane (10 mL, 113.471 mmol, 87.69 equiv) was stirred overnight at 80° C. under nitrogen atmosphere. The mixture was allowed to cool down to room temperature, then quenched with sat. KF (aq.) (30 mL) at 0° C. The resulting mixture was extracted with ethyl acetate (3×30 mL). The combined organic layers were washed with brine (2×20 mL), dried over anhydrous Na₂SO₄, and filtered. After filtration, the filtrate was concentrated under reduced pressure to give a residue. The residue (tert-butyl 4-[5-(trimethylstannyl)thieno[2,3-c]pyrazol-2-yl]piperidine-1-carboxylate (950 mg, 78.05%)) was used in the next step without further purification.

### Synthesis of Intermediate B135

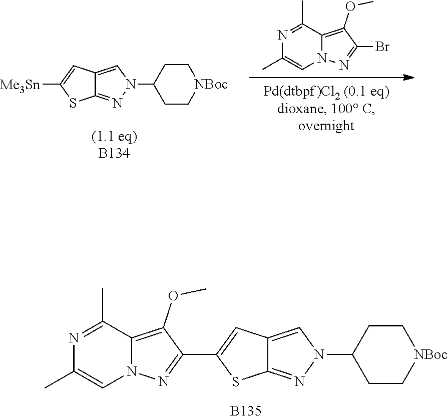

A mixture of 2-bromo-3-methoxy-4,6-dimethylpyrazolo[1,5-a]pyrazine (100 mg, 0.390 mmol, 1.00 equiv), tert-butyl 4-[5-(trimethylstannyl)thieno[2,3-c]pyrazol-2-yl]piperidine-1-carboxylate (201.97 mg, 0.429 mmol, 1.1 equiv), and Pd(DtBPF)Cl₂ (25.45 mg, 0.039 mmol, 0.1 equiv) in 1,4-dioxane (5 mL, 56.750 mmol, 145.34 equiv) was stirred overnight at 100° C. under nitrogen atmosphere. The resulting mixture was concentrated under reduced pressure to give a residue. The residue was purified by silica gel column chromatography, eluted with DCM/EA (2:1) to afford tert-butyl 4-(5-{3-methoxy-4,6-dimethylpyrazolo[1,5-a]pyrazin-2-yl}thieno [2,3-c]pyrazol-2-yl)piperidine-1-carboxylate (125 mg, 66.33%) as a solid.

### Synthesis of Compound 213

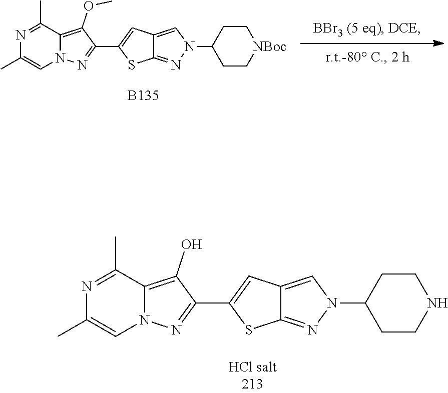 [SMILES: "B135" ; COc1c(-c2cc3cn(C4CCN(C(=O)OC(C)(C)C)CC4)nc3s2)nn2cc(C)nc(C)c12 ; Cc1cn2nc(-c3cc4cn(C5CCNCC5)nc4s3)c(O)c2c(C)n1]

To a stirred solution of tert-butyl 4-(5-{3-methoxy-4,6-dimethylpyrazolo[1,5-a]pyrazin-2-yl}thieno [2,3-c]pyrazol-2-yl)piperidine-1-carboxylate (50 mg, 0.104 mmol, 1.00 equiv) in DCE (1 mL) was added BBr₃ (129.78 mg, 0.520 mmol, 5 equiv) (in 1 mL DCE) dropwise at room temperature under air atmosphere. The resulting mixture was stirred for an additional 2 h at 80° C., then quenched with MeOH (5 mL) at 0° C. The resulting mixture was concentrated under reduced pressure to give a residue. The residue was purified by Prep-HPLC (Column: Xselect CSH C18 OBD Column 30×150 mm 5 m, n; Mobile Phase A: Water (0.05% HCl), Mobile Phase B: ACN; Flow rate: 60 mL/min; Gradient: 3% B to 43% B in 8 min; Wave Length: 220 nm; RT1(min): 5.24.) to afford 4,6-dimethyl-2-[2-(piperidin-4-yl)thieno[2,3-c]pyrazol-5-yl]pyrazolo[1,5-a]pyrazin-3-ol hydrochloride (20.7 mg, 48.95%) as a solid.

Compounds 213 and 215 were prepared according to the procedures described herein and outlined in this Example 61. The table below provides intermediates used in these procedures and final compound characterization data.

| | | LCMS | |
|---|---|---|---|
| | | (ESI, | |
| | | m/z) | |
| Compound No. and Structure | Coupling Reagent | [M + H]⁺ | ¹H NMR δ |
| 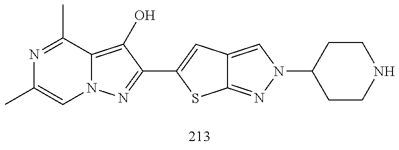 [SMILES: Cc1cn2nc(-c3cc4cn(C5CCNCC5)nc4s3)c(O)c2c(C)n1] | 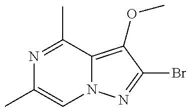 | 369 | (400 MHz, 353K, DMSO- d₆): δ 9.23 (s, 1H), 9.06 (s, 1H), 8.34 (s, 1H), 8.11 (s, 1H), 7.68 (s, 1H), 4.69 (tt, J = 9.1, 5.6 Hz, 1H), 3.42 (d, J = 13.0 Hz, 2H), 3.11 (d, J = 11.0 Hz, 2H), 2.90 (s, 3H), 2.41 (s, 3H), 2.36- |
| | | | 2.26 (m, 4H) |
| 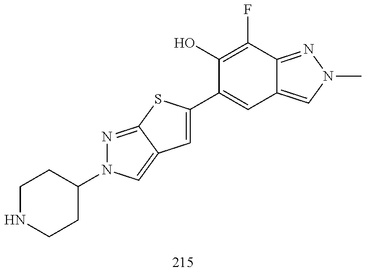 [SMILES: Cn1cc2cc(-c3cc4cn(C5CCNCC5)nc4s3)c(O)c(F)c2n1] | 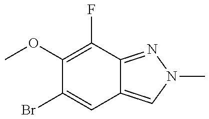 | 372 | (400 MHz, DMSO-d₆): δ 8.36 (d, J = 2.7 Hz, 1H), 8.07 (s, 1H), 7.67 (d, J = 0.9 Hz, 1H), 7.48 (s, 1H), 4.36 (tt, J = 11.5, 4.1 Hz, 1H), 4.14 (s, 3H), 3.07 (d, J = 12.1 Hz, 2H), 2.70- 2.58 (m, 2H), 2.07-1.96 (m, 2H), 1.89 (qd, J = 12.0, 4.1 Hz, 2H) |

### Example 62: Synthesis of Compound 134

### Synthesis of Intermediate B136

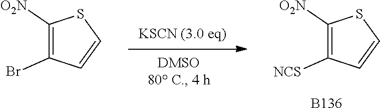

A mixture of 3-bromo-2-nitrothiophene (80.00 g, 384.56 mmol, 1.00 equiv), DMSO (250.00 mL), and potassium thiocyanate (112.00 g, 3.00 equiv) was stirred for 4 h at 80° C. The resulting solution was extracted with ethyl acetate (3×200 mL). The organic layers were combined, washed with saturated NaCl (1×200 mL), dried over anhydrous sodium sulfate, and concentrated under vacuum to afford [(2-nitrothiophen-3-yl)sulfanyl]formonitrile as a solid (68 g, 94.97%). LCMS (ES, m/z):187 [M+H]⁺.

### Synthesis of Intermediate B137

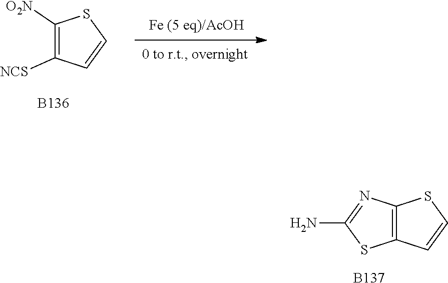

A mixture of [(2-nitrothiophen-3-yl)sulfanyl]formonitrile (68.00 g, 365.20 mmol, 1.00 equiv), AcOH (1.50 L), and Fe (101.97 g, 1825.99 mmol, 5.00 equiv) was stirred for 8 h at room temperature. The reaction mixture was filtered to remove solids, and the filtrate extracted with ethyl acetate (3×1 L). The organic layers were combined, washed with saturated NaCl (1 L), dried over anhydrous sodium sulfate, and concentrated under vacuum to give a residue. The residue was purified by silica gel column chromatograpy, eluted with ethyl acetate/petroleum ether (1:5) to afford thieno[2,3-d][1,3]thiazol-2-amine (50.00 g, 87.64%) as a solid. LCMS (ES, m/z):157 [M+H]⁺.

### Synthesis of Intermediate B138

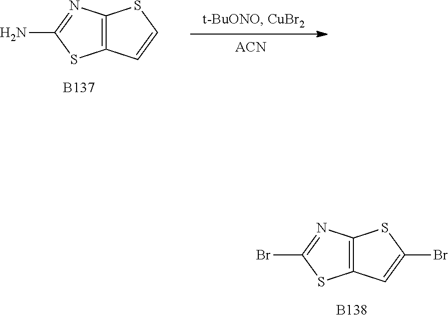

A mixture of t-BuNO₂ (9.90 g, 0.096 mmol, 1.50 equiv), CuBr₂ (14.30 g, 0.06 mmol, 1.00 equiv), ACN (200.00 mL), and thieno[2,3-d][1,3]thiazol-2-amine (10.00 g, 64.01 mmol, 1.00 equiv) was stirred for 1 h at 65° C. The reaction mixture was quenched with HCl (50 mL, 6 M), then extracted with Et₂O (3×100 mL). The organic layers were combined, dried over anhydrous sodium sulfate, filtered, and the filtrate concentrated under vacuum to give a residue. The residue was purified by silica gel column chromatography, eluted with ethyl acetate/petroleum ether (1:10) to afford 2,5-dibromothieno[2,3-d][1,3]thiazole (8 g, 41.80%) as a solid. LCMS (ES, m/z):298 [M+H]⁺.

### Synthesis of Intermediate B139

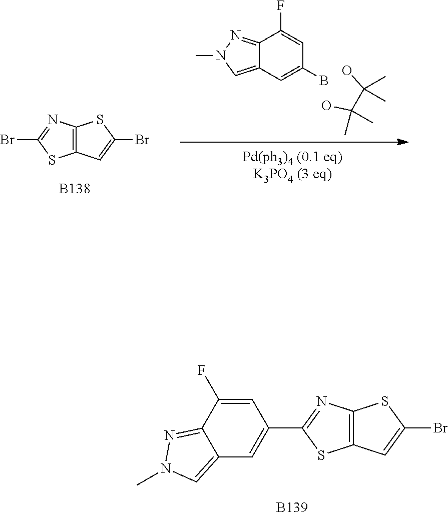

A mixture of 2,5-dibromothieno[2,3-d][1,3]thiazole (3.00 g, 10.03 mmol, 1.00 equiv), K₃PO₄ (6.39 g, 30.10 mmol, 3.00 equiv), dioxane/H₂O (5:1, 30.00 mL), Pd(PPh₃)₄(1.16 g, 1.00 mmol, 0.10 equiv), and 7-fluoro-2-methyl-5-(4,4,5,5-tetramethyl-1,3,2-dioxaborolan-2-yl)indazole (3.05 g, 11.04 mmol, 1.10 equiv) was stirred for 16 h at 80° C. The resulting solution was extracted with ethyl acetate (3×30 mL). The organic layers combined, washed with saturated NaCl (1×30 mL), dried over anhydrous sodium sulfate, filtered, and the filtrate concentrated under vacuum to give a residue. The residue was purified by silica gel column chromatography, eluted with ethyl acetate/petroleum ether (1:4) to afford 5-[5-bromothieno[2,3-d][1,3]thiazol-2-yl]-7-fluoro-2-methylindazole as a brown solid. LCMS (ES, m/z):368 [M+H]⁺.

### Synthesis of Intermediate B140

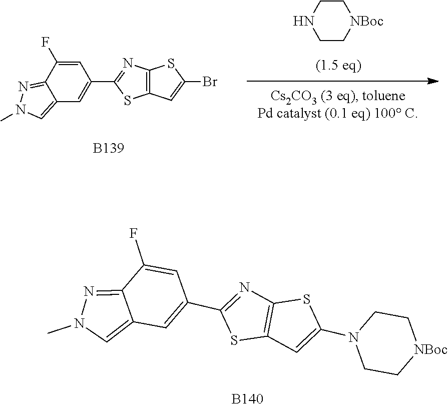 [SMILES: "B139" ; "B140" ; Cn1cc2cc(-c3nc4sc(Br)cc4s3)cc(F)c2n1 ; Cn1cc2cc(-c3nc4sc(N5CCN(C(=O)OC(C)(C)C)CC5)cc4s3)cc(F)c2n1 ; CC(C)(C)OC(=O)N1CCNCC1]

A mixture of 5-[5-bromothieno[2,3-d][1,3]thiazol-2-yl]-7-fluoro-2-methylindazole (50.00 mg, 0.14 mmol, 1.00 equiv), tert-butyl piperazine-1-carboxylate (37.93 mg, 0.20 mmol, 1.50 equiv), Pd-PEPPSI-IPentCl 2-methylpyridine (o-picoline) (11.42 mg, 0.01 mmol, 0.10 equiv), Cs₂CO₃ (132.72 mg, 0.41 mmol, 3.00 equiv), and toluene (3.00 mL) was stirred for 10 h at 100° C. The resulting solution was extracted with ethyl acetate (3×10 mL). The organic layers were combined, washed with saturated NaCl (1×10 mL), dried over anhydrous sodium sulfate, and filtered. The filtrate was concentrated under vacuum to give a residue. The residue was applied onto a silica gel column with ethyl acetate/petroleum ether (1:4) to afford tert-butyl 4-[2-(7-fluoro-2-methylindazol-5-yl)thieno[2,3-d][1,3]thiazol-5-yl]piperazine-1-carboxylate (40.00 mg, 62.20%) as a solid. LCMS (ES, m/z): 473 [M+H]⁺.

### Synthesis of Compound 134

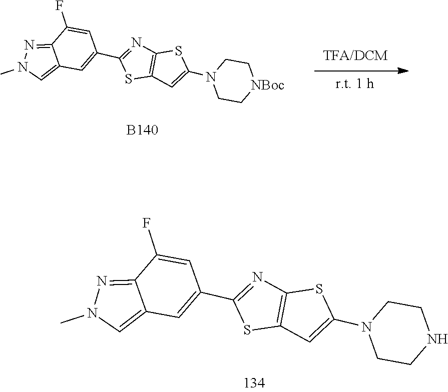 [SMILES: "B140" ; Cn1cc2cc(-c3nc4sc(N5CCN(C(=O)OC(C)(C)C)CC5)cc4s3)cc(F)c2n1 ; Cn1cc2cc(-c3nc4sc(N5CCNCC5)cc4s3)cc(F)c2n1]

A mixture of tert-butyl 4-[2-(7-fluoro-2-methylindazol-5-yl)thieno[2,3-d][1,3]thiazol-5-yl]piperazine-1-carboxylate (40.00 mg, 0.08 mmol, 1.00 equiv) and TFA/DCM (5.00 mL) was stirred for 1 h at room temperature. The resulting mixture was concentrated under vacuum to give a residue. The residue was purified by Prep-HPLC ((Condition 2, Gradient 11) to afford 7-fluoro-2-methyl-5-[5-(piperazin-1-yl)thieno[2,3-d][1,3]thiazol-2-yl]indazole (11.10 mg, 35.19%) as a solid. LCMS (ES, m/z):373 [M+H]⁺. ¹H NMR (400 MHz, DMSO-d₆) δ 8.57 (d, J=2.8 Hz, 1H), 8.09 (d, J=1.3 Hz, 1H), 7.59 (dd, J=12.6, 1.4 Hz, 1H), 6.50 (s, 1H), 4.23 (s, 3H), 3.10 (dd, J=6.3, 3.9 Hz, 4H), 2.86 (dd, J=6.2, 3.9 Hz, 4H).

### Example 63: Synthesis of Compound 141

### Synthesis of Compound 141

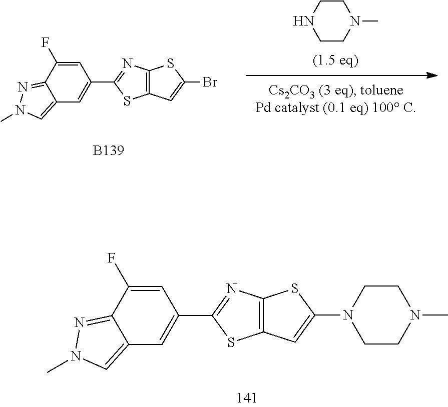

Into a 8-mL sealed tube purged and maintained with an inert atmosphere of nitrogen, 5-[5-bromothieno[2,3-d][1,3]thiazol-2-yl]-7-fluoro-2-methylindazole (50.00 mg, 0.14 mmol, 1.00 equiv), piperazine, 1-methyl-(20.40 mg, 0.20 mmol, 1.50 equiv), Pd-PEPPSI-IPentCl 2-methylpyridine (o-picoline) (11.42 mg, 0.01 mmol, 0.10 equiv), Cs₂CO₃ (132.72 mg, 0.41 mmol, 3.00 equiv), and toluene were combined. The resulting solution was stirred for 10 h at 100° C., then extracted with ethyl acetate (3×10 mL). The organic layers were combined, washed with saturated NaCl (1×10 mL), dried over anhydrous sodium sulfate, and concentrated under vacuum to give a residue. The residue was purified by Prep-HPLC (Condition 2, Gradient 12) to afford 7-fluoro-2-methyl-5-[5-(4-methylpiperazin-1-yl)thieno[2,3-d][1,3]thiazol-2-yl]indazole (2.80 mg, 5.32%) as a solid. LCMS (ES, m/z):388 [M+H]⁺. 1H NMR (400 MHz, DMSO-d₆) δ 8.57 (d, J=2.8 Hz, 1H), 8.10 (d, J=1.3 Hz, 1H), 7.59 (dd, J=12.7, 1.4 Hz, 1H), 6.53 (s, 1H), 4.23 (s, 3H), 3.20 (t, J=5.1 Hz, 4H), 3.13 (t, J=5.1 Hz, 4H), 2.25 (s, 3H).

### Example 64: Synthesis of Compound 142

### Synthesis of Intermediate B141

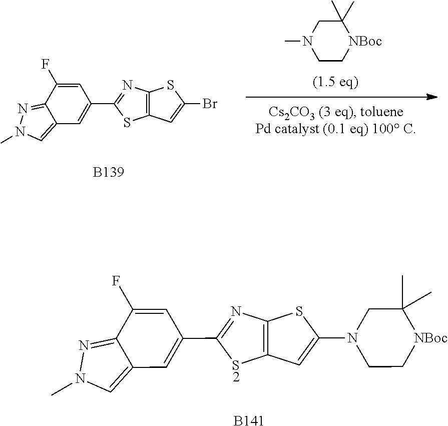

A mixture of 5-[5-bromothieno[2,3-d][1,3]thiazol-2-yl]-7-fluoro-2-methylindazole (50.00 mg, 0.14 mmol, 1.00 equiv), tert-butyl 2,2-dimethylpiperazine-1-carboxylate (43.65 mg, 0.20 mmol, 1.50 equiv), Pd-PEPPSI-IPentCl 2-methylpyridine (o-picoline (11.42 mg, 0.01 mmol, 0.10 equiv), Cs₂CO₃ (132.72 mg, 0.40 mmol, 3.00 equiv), and toluene (3.00 mL) was stirred for 10 h at 100° C. The resulting solution was extracted with ethyl acetate (3×10 mL). The organic layers were combined, washed with saturated NaCl (1×10 mL), dried over anhydrous sodium sulfate, filtered, and the filtrate concentrated under reduced pressure to give a residue. The residue was purified by silica gel column chromatography with ethyl acetate/petroleum ether (1:4) to afford tert-butyl 4-[2-(7-fluoro-2-methylindazol-5-yl)thieno[2,3-d][1,3]thiazol-5-yl]-2,2-dimethylpiperazine-1-carboxylate (42 mg, 61.66%) as a solid. LCMS (ES, m/z):502 [M+H]⁺.

### Synthesis of Compound 142

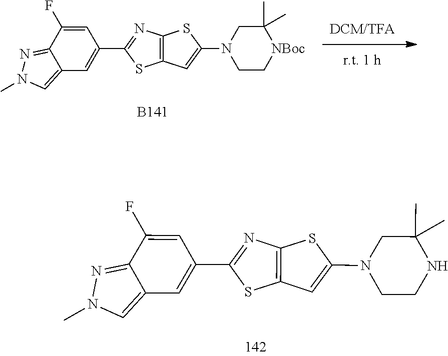

A mixture of tert-butyl 4-[2-(7-fluoro-2-methylindazol-5-yl)thieno[2,3-d][1,3]thiazol-5-yl]-2,2-dimethylpiperazine-1-carboxylate (42.00 mg, 0.08 mmol, 1.00 equiv) and TFA/DCM (5.00 mL) was stirred for 1 h at room temperature, then was concentrated under vacuum to give a residue. The residue was purified by Prep-HPLC (Condition 2, Gradient 13) to afford 5-[5-(3,3-dimethylpiperazin-1-yl)thieno[2,3-d][1,3]thiazol-2-yl]-7-fluoro-2-methylindazole (16.70 mg, 49.68%) as a solid. LCMS (ES, m/z): 402 [M+H]⁺. ¹H NMR (400 MHz, DMSO-d₆) δ 8.56 (d, J=2.8 Hz, 1H), 8.08 (d, J=1.3 Hz, 1H), 7.59 (dd, J=12.6, 1.4 Hz, 1H), 6.49 (s, 1H), 4.23 (s, 3H), 3.06 (dd, J=6.1, 4.3 Hz, 2H), 2.89 (d, J=4.6 Hz, 4H), 2.03 (s, 1H), 1.12 (s, 6H).

### Example 65: Synthesis of Compound 146

### Synthesis of Compound 146

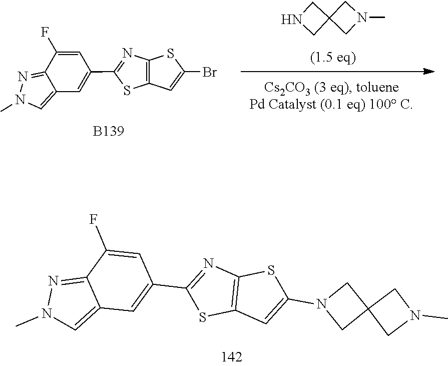

A mixture of 5-[5-bromothieno[2,3-d][1,3]thiazol-2-yl]-7-fluoro-2-methylindazole (50.00 mg, 0.14 mmol, 1.00 equiv), 2-methyl-2,6-diazaspiro[3.3]heptane (22.85 mg, 0.20 mmol, 1.50 equiv), Pd-PEPPSI-IPentCl 2-methylpyridine (o-picoline (11.42 mg, 0.01 mmol, 0.10 equiv), Cs₂CO₃ (132.72 mg, 0.41 mmol, 3.00 equiv), and toluene (3.00 mL) was stirred for 10 h at 100° C. The resulting solution was extracted with ethyl acetate (3×10 mL). The organic layers were combined, washed with saturated NaCl (1×10 mL), dried over anhydrous sodium sulfate, filtered, and the filtrate concentrated under vacuum to give a residue. The residue was purified by Prep-HPLC (Condition 2, Gradient 13) to afford 7-fluoro-2-methyl-5-(5-[6-methyl-2,6-diazaspiro[3.3]heptan-2-yl]thieno[2,3-d][1,3]thiazol-2-yl)indazole (20.90 mg, 38.53%) as a solid. LCMS (ES, m/z): 400 [M+H]⁺. ¹H NMR (400 MHz, DMSO-d₆) δ 8.56 (d, J=2.8 Hz, 1H), 8.07 (d, J=1.4 Hz, 1H), 7.58 (dd, J=12.6, 1.4 Hz, 1H), 6.22 (s, 1H), 4.22 (s, 3H), 3.98 (s, 4H), 3.31 (s, 4H), 2.19 (s, 3H).

### Example 66: Synthesis of Compound 143

### Synthesis of Compound 143

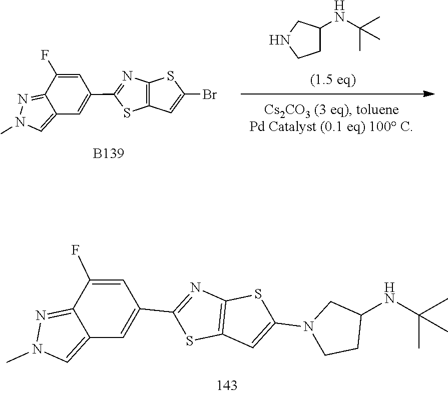

A mixture of 5-[5-bromothieno[2,3-d][1,3]thiazol-2-yl]-7-fluoro-2-methylindazole (100.00 mg, 0.27 mmol, 1.00 equiv), N-tert-butylpyrrolidin-3-amine (57.94 mg, 0.41 mmol, 1.50 equiv), Pd-PEPPSI-IPentCl 2-methylpyridine (o-picoline (22.84 mg, 0.03 mmol, 0.10 equiv), Cs₂CO₃ (265.44 mg, 0.82 mmol, 3.00 equiv), and toluene (3 mL) was stirred for 10 h at 100° C. The resulting solution was extracted with ethyl acetate (3×10 mL). The organic layers were combined, washed with saturated NaCl (1×10 mL), dried over anhydrous sodium sulfate, and concentrated under vacuum to give a residue. The residue was purified by silica gel column chromatography, eluted with ethyl acetate/petroleum ether (1:4), followed by Prep-HPLC Condition 1, Gradient 5) to afford N-tert-butyl-1-[2-(7-fluoro-2-methylindazol-5-yl)thieno[2,3-d][1,3]thiazol-5-yl]pyrrolidin-3-amine (13.00 mg, 11.14%) as a solid. LCMS (ES, m/z):430 [M+H]+. ¹H NMR (400 MHz, DMSO-d₆) δ 8.54 (d, J=2.8 Hz, 1H), 8.02 (d, J=1.3 Hz, 1H), 7.57 (dd, J=12.7, 1.4 Hz, 1H), 4.22 (s, 3H), 3.27 (td, J=8.9, 7.1 Hz, 4H), 2.94 (t, J=7.9 Hz, 4H).

### Example 67: Synthesis of Compound 144

### Synthesis of Compound 144

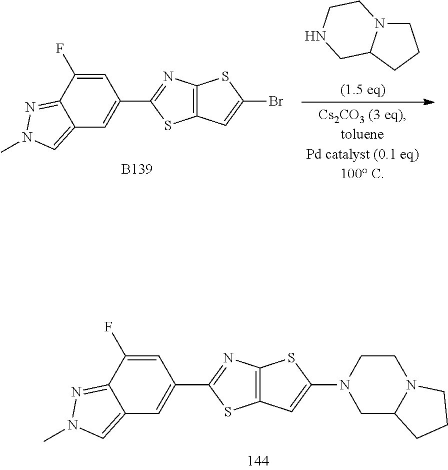 [SMILES: "B139" ; Cn1cc2cc(-c3nc4sc(Br)cc4s3)cc(F)c2n1 ; Cn1cc2cc(-c3nc4sc(N5CCN6CCCC6C5)cc4s3)cc(F)c2n1 ; C1CC2CNCCN2C1]

A mixture of 5-[5-bromothieno[2,3-d][1,3]thiazol-2-yl]-7-fluoro-2-methylindazole (100.00 mg, 0.27 mmol, 1.00 equiv), octahydropyrrolo[1,2-a]pyrazine (51.41 mg, 0.41 mmol, 1.50 equiv), Pd-PEPPSI-IPentCl 2-methylpyridine (o-picoline (22.84 mg, 0.03 mmol, 0.10 equiv), Cs₂CO₃ (265.44 mg, 0.82 mmol, 3.00 equiv), and toluene (3.00 mL) was stirred for 10 h at 100° C. The resulting solution was extracted with ethyl acetate (3×10 mL). The organic layers were combined, washed with saturated NaCl (1×10 mL), filtered, and the filtrate concentrated under reduced pressure to give a residue. The residue was purified by silica gel column chromatography eluted with ethyl acetate/petroleum ether (1:4), followed by Prep-HPLC (Condition 1, Gradient 5) to afford 7-fluoro-5-(5-[hexahydro-1H-pyrrolo[1,2-a]pyrazin-2-yl]thieno[2,3-d][1,3]thiazol-2-yl)-2-methylindazole (35.70 mg, 31.79%) as a solid. LCMS (ES, m/z):414 [M+H]⁺. ¹H NMR (400 MHz, DMSO-d₆) δ 8.57 (d, J=2.7 Hz, 1H), 8.09 (d, J=1.3 Hz, 1H), 7.59 (dd, J=12.7, 1.4 Hz, 1H), 6.51 (s, 1H), 4.22 (s, 3H), 3.66 (ddd, J=11.1, 3.2, 1.2 Hz, 1H), 3.54-3.46 (m, 1H), 3.12-2.99 (m, 2H), 2.96 (td, J=11.6, 3.4 Hz, 1H), 2.64 (t, J=10.7 Hz, 1H), 2.28 (td, J=11.3, 3.3 Hz, 1H), 2.16-2.04 (m, 2H), 1.92-1.81 (m, 1H), 1.72 (s, 2H), 1.39 (qd, J=11.0, 6.9 Hz, 1H).

### Example 68: Synthesis of Compound 145

### Synthesis of Compound 145

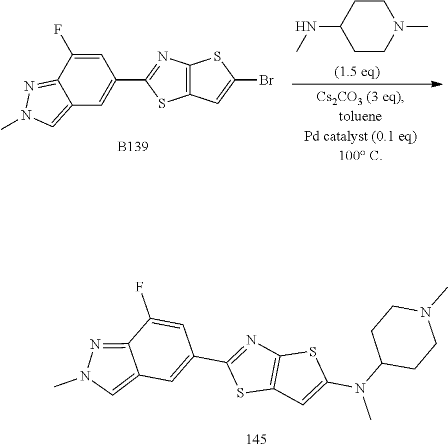

A mixture of 5-[5-bromothieno[2,3-d][1,3]thiazol-2-yl]-7-fluoro-2-methylindazole (50.00 mg, 0.14 mmol, 1.00 equiv), N,1-dimethylpiperidin-4-amine (26.11 mg, 0.00 mmol, 1.50 equiv), Pd-PEPPSI-IPentCl 2-methylpyridine (o-picoline (11.42 mg, 0.01 mmol, 0.10 equiv), Cs₂CO₃ (132.72 mg, 0.41 mmol, 3.00 equiv), and toluene (3.00 mL). The resulting solution was stirred for 10 h at 100° C., then extracted with ethyl acetate (3×10 mL). The organic layers were combined, washed with saturated NaCl (1×10 mL), dried over anhydrous sodium sulfate, filtered, and the filtrate concentrated under vacuum to give a residue. The residue was purified by Prep-HPLC (Condition 5, Gradient 18) to afford N-[2-(7-fluoro-2-methylindazol-5-yl)thieno[2,3-d][1,3]thiazol-5-yl]-N,1-dimethylpiperidin-4-amine (5.20 mg, 9.22%) as a solid. LCMS (ES, m/z):416 [M+H]⁺. ¹H NMR (400 MHz, DMSO-d₆) δ 8.55 (d, J=2.7 Hz, 1H), 8.06 (d, J=1.3 Hz, 1H), 7.58 (dd, J=12.7, 1.3 Hz, 1H), 6.33 (s, 1H), 4.22 (s, 3H), 3.30 (s, 1H), 2.88 (s, 3H), 2.83 (s, 3H), 2.22 (s, 3H), 2.02 (s, 3H), 1.79 (d, J=11.3 Hz, 1H), 1.73 (s, 4H).

### Example 69: Synthesis of Compounds 192 and 193

### Synthesis of Intermediate B142

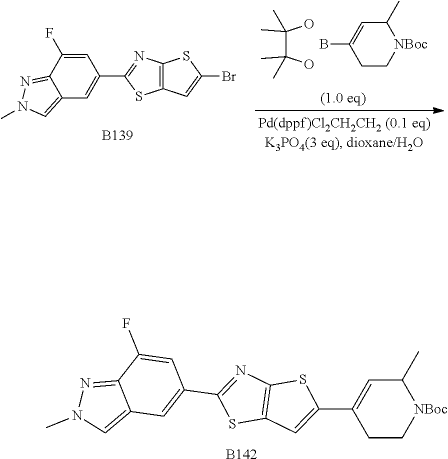

A mixture of5-[5-bromothieno[2,3-d][1,3]thiazol-2-yl]-7-fluoro-2-methylindazole (100.00 mg, 0.27 mmol, 1.00 equiv), dioxane/H₂O (5.00 mL), K₃PO₄ (172.93 mg, 0.82 mmol, 3.00 equiv), Pd(dppf)Cl₂ CH₂Cl₂ (22.12 mg, 0.03 mmol, 0.10 equiv), and tert-butyl 2-methyl-4-(4,4,5,5-tetramethyl-1,3,2-dioxaborolan-2-yl)-5,6-dihydro-2H-pyridine-1-carboxylate (96.56 mg, 0.30 mmol, 1.10 equiv) was stirred for 8 h at 80° C. The resulting solution was extracted with ethyl acetate (3×10 mL). The organic layers were combined, washed with saturated NaCl (3×10 mL), dried over anhydrous sodium sulfate, filtered and the filtrate concentrated under vacuum to give a residue. The residue was purified by silica gel column chromatography with ethyl acetate/petroleum ether (1:5) to afford tert-butyl 4-[2-(7-fluoro-2-methylindazol-5-yl)thieno[2,3-d][1,3]thiazol-5-yl]-2-methyl-5,6-dihydro-2H-pyridine-1-carboxylate (87.00 mg, 66.11%) as a solid. LCMS (ES, m/z):485 [M+H]⁺.

### Synthesis of Intermediate B143

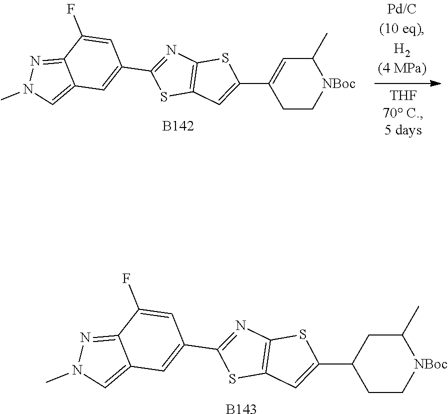 [SMILES: "B142" ; "B143" ; CC1C=C(c2cc3sc(-c4cc(F)c5nn(C)cc5c4)nc3s2)CCN1C(=O)OC(C)(C)C ; CC1CC(c2cc3sc(-c4cc(F)c5nn(C)cc5c4)nc3s2)CCN1C(=O)OC(C)(C)C]

A mixture of tert-butyl 4-[2-(7-fluoro-2-methylindazol-5-yl)thieno [2,3-d][1,3]thiazol-5-yl]-2-methyl-5,6-dihydro-2H-pyridine-1-carboxylate (87.00 mg, 1.00 equiv), THE (5.00 mL), and Pd(OH)₂/C (20.00 mg) was stirred for 5 days at 70° C. under H₂ (4 MPa). The reaction mixture was filtered to remove solids. The filtrate was concentrated under vacuum to afford tert-butyl 4-[2-(7-fluoro-2-methylindazol-5-yl)thieno[2,3-d][1,3]thiazol-5-yl]-2-methylpiperidine-1-carboxylate (65.00 mg, 74.40%) as a solid. LCMS (ES, m/z): 487 [M+H]⁺.

### Synthesis of Compounds 193 and 194

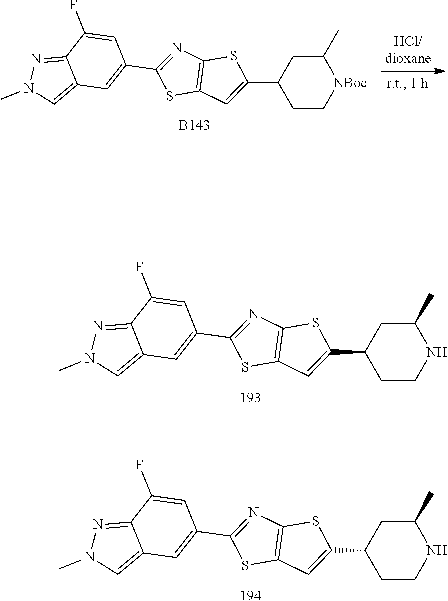

A solution of tert-butyl 4-[2-(7-fluoro-2-methylindazol-5-yl)thieno[2,3-d][1,3]thiazol-5-yl]-2-methylpiperidine-1-carboxylate (65.00 mg, 0.13 mmol, 1.00 equiv)] in HCl (5 mL) was stirred for 1 h at room temperature, then basified to pH 8 with saturated NaHCO₃ (aq.). The resulting mixture was extracted with ethyl acetate (3×10 mL). The organic layers were combined, washed with saturated NaCl (1×10 mL), dried over anhydrous Na₂SO₄, and filtered. After filtration, the filtrate was concentrated under reduced pressure to give a residue. The residue was purified by Prep-chiral-HPLC (Condition 3, Gradient 1) to afford 7-fluoro-2-methyl-5-[5-(2-methylpiperidin-4-yl)thieno[2,3-d][1,3]thiazol-2-yl]indazole (8.10 mg, 32.1%) and 7-fluoro-2-methyl-5-[5-(2-methylpiperidin-4-yl)thieno 2,3-d][1,3]thiazol-2-yl]indazole (3.70 mg, 11.09%) as solids. Compound 193: LCMS (ES, m/z):387 [M+H]⁺. ¹H NMR (400 MHz, DMSO-d₆) δ 8.54 (s, 1H), 8.15 (s, 1H), 7.59 (d, J=12.4 Hz, 1H), 7.18 (s, 1H), 4.19 (s, 3H), 2.97 (d, J=12.3 Hz, 2H), 2.60 (t, J=11.7 Hz, 2H), 1.89 (t, J=12.2 Hz, 2H), 1.53-1.40 (m, 1H), 1.18 (q, J=11.9 Hz, 1H), 1.01 (d, J=6.2 Hz, 3H). Compound 194: LCMS (ES, m/z):387 [M+H]+. ¹H NMR (400 MHz, DMSO-d₆) δ 8.61 (d, J=2.8 Hz, 1H), 8.22 (s, 1H), 7.64 (d, J=12.5 Hz, 1H), 7.32 (s, 1H), 4.24 (s, 3H), 3.41(td, J=7.0, 3.3 Hz, 1H), 3.00 (dtt, J=20.9, 8.1, 4.5 Hz, 1H), 2.79(dtt, J=20.9, 8.1, 4.5 Hz, 2H)1.90 (tt, J=8.5, 4.4 Hz, 2H), 1.75 (d, J=13.6 Hz, 1H), 1.65 (ddd, J=12.6, 7.5, 4.5 Hz, 1H), 1.06 (d, J=6.5 Hz, 3H).

### Example 70: Synthesis of Compound 247

### Synthesis of Compound 247

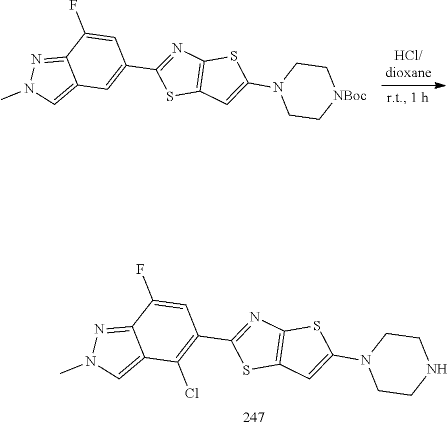 [SMILES: Cn1cc2cc(-c3nc4sc(N5CCN(C(=O)OC(C)(C)C)CC5)cc4s3)cc(F)c2n1 ; Cn1cc2c(Cl)c(-c3nc4sc(N5CCNCC5)cc4s3)cc(F)c2n1]

A mixture of tert-butyl 4-[2-(7-fluoro-2-methylindazol-5-yl)thieno[2,3-d][1,3]thiazol-5-yl]piperazine-1-carboxylate (40.00 mg, 0.08 mmol, 1.00 equiv) and HCl (gas) in 1,4-dioxane (3.00 mL) was stirred for 1 h at room temperature. The resulting mixture was concentrated under vacuum to give a residue. The residue was purified by Prep-HPLC (Condition 2, Gradient 13) to afford 4-chloro-7-fluoro-2-methyl-5-[5-(piperazin-1-yl)thieno[2,3-d][1,3]thiazol-2-yl]indazole (2.60 mg, 7.55%) as a solid. LCMS (ES, m/z):408 [M+H]⁺. ¹H NMR (400 MHz, DMSO-d₆) δ 8.63 (d, J=2.8 Hz, 1H), 8.25 (d, J=1.4 Hz, 1H), 7.64 (dd, J=12.5, 1.4 Hz, 1H), 4.24 (s, 3H), 3.02 (dd, J=5.8, 3.8 Hz, 4H), 2.88 (t, J=4.9 Hz, 4H).

### Example 71: Synthesis of Compound 194

### Synthesis of Intermediate B144

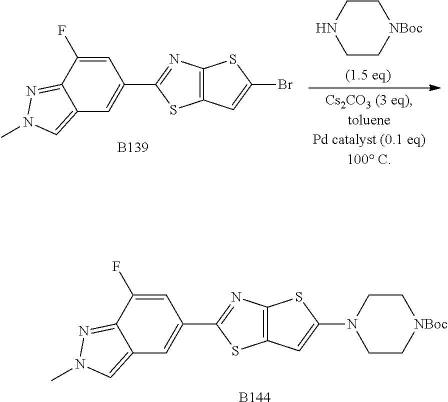

To a stirred mixture of 5-{5-bromothieno[2,3-d][1,3]thiazol-2-yl}-7-fluoro-2-methylindazole (200.0 mg, 0.54 mmol, 1.00 equiv) and tert-butyl piperazine-1-carboxylate (151.7 mg, 0.82 mmol, 1.50 equiv) in toluene (5 mL) was added Pd-PEPPSI-IPentCl₂-methylpyridine-o-picoline (45.6 mg, 0.05 mmol, 0.10 equiv) and Cs₂CO₃ (530.8 mg, 1.63 mmol, 3.00 equiv). The reaction mixture was stirred for 8 h at 100° C. under nitrogen atmosphere, then extracted with ethyl acetate (3×10 mL). The organic layers were combined, washed with saturated NaCl (1×10 mL), dried over anhydrous Na₂SO₄, and filtered. After filtration, the filtrate was concentrated under reduced pressure to give a residue. The residue was purified by silica gel column chromatography, eluted with CH₂Cl₂/MeOH (10:1) to afford tert-butyl 4-[2-(7-fluoro-2-methylindazol-5-yl)thieno [2,3-d][1,3] thiazol-5-yl]piperazine-1-carboxylate (160 mg, 62.2%) as a solid. LCMS (ES, m/z):474 [M+H]⁺.

### Synthesis of Intermediate B145

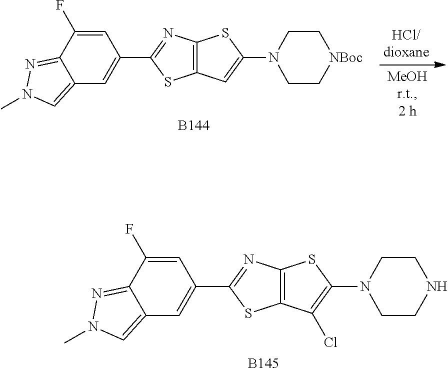 [SMILES: "B144" ; Cn1cc2cc(-c3nc4sc(N5CCN(C(=O)OC(C)(C)C)CC5)cc4s3)cc(F)c2n1 ; "B145" ; Cn1cc2cc(-c3nc4sc(N5CCNCC5)c(Cl)c4s3)cc(F)c2n1]

A solution of tert-butyl 4-[2-(7-fluoro-2-methylindazol-5-yl)thieno[2,3-d][1,3]thiazol-5-yl]piperazine-1-carboxylate (160 mg, 0.34 mmol, 1.00 equiv) in HCl (gas) in 1,4-dioxane (5 mL, 164.56 mmol, 487.09 equiv) was stirred for 1 h at room temperature. The resulting mixture was concentrated under reduced pressure to afford 5-[6-chloro-5-(piperazin-1-yl)thieno[2,3-d][1,3]thiazol-2-yl]-7-fluoro-2-methy-lindazole (100.0 mg, 72.56%) as a solid. LCMS (ES, m/z):408 [M+H]⁺.

### Synthesis of Intermediate B146

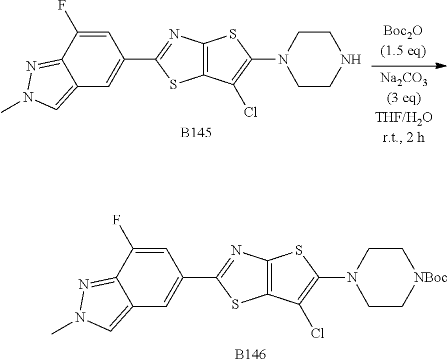 [SMILES: "B145" ; "B146" ; Cn1cc2cc(-c3nc4sc(N5CCNCC5)c(Cl)c4s3)cc(F)c2n1 ; Cn1cc2cc(-c3nc4sc(N5CCN(C(=O)OC(C)(C)C)CC5)c(Cl)c4s3)cc(F)c2n1]

To a stirred mixture of 5-[6-chloro-5-(piperazin-1-yl)thieno[2,3-d][1,3]thiazol-2-yl]-7-fluoro-2-methylindazole (100.0 mg, 0.25 mmol, 1.00 equiv) and Boc₂0 (80.2 mg, 0.37 mmol, 1.50 equiv) in THF/water (1:1) (5 mL) was added Na₂CO₃ (77.95 mg, 0.74 mmol, 3.00 equiv) in portions. The reaction mixture was stirred for 2 h at room temperature. The resulting mixture was extracted with ethyl acetate (3×10 mL). The organic layers were combined, washed with saturated NaCl (1×10 mL), dried over anhydrous Na₂SO₄, and filtered. After filtration, the filtrate was concentrated under reduced pressure to afford tert-butyl 4-[6-chloro-2-(7-fluoro-2-methylindazol-5-yl)thieno[2,3-d][1,3]thiazol-5-yl]piperazine-1-carboxylate (80.00 mg, 64.23%) as a solid. LCMS (ES, m/z):508 [M+H]

### Synthesis of Intermediate B147

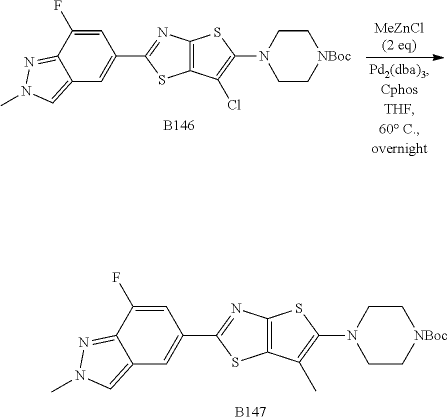 [SMILES: "B146" ; "B147" ; Cn1cc2cc(-c3nc4sc(N5CCN(C(=O)OC(C)(C)C)CC5)c(Cl)c4s3)cc(F)c2n1 ; Cc1c(N2CCN(C(=O)OC(C)(C)C)CC2)sc2nc(-c3cc(F)c4nn(C)cc4c3)sc12]

To a stirred mixture of tert-butyl 4-[6-chloro-2-(7-fluoro-2-methylindazol-5-yl)thieno[2,3-d][1,3]thiazol-5-yl]piperazine-1-carboxylate (80.0 mg, 0.16 mmol, 1.00 equiv) and chloro(methyl)zinc (36.5 mg, 0.31 mmol, 2.00 equiv) in THF (5 ml) was added CPhos (6.8 mg, 0.02 mmol, 0.10 equiv) and Pd₂(dba)₃ (16.3 mg, 0.02 mmol, 0.10 equiv) in portions. The reaction mixture was stirred for 8 h at 60° C. under nitrogen atmosphere, then extracted with ethyl acetate (3×10 mL). The organic layers were combined, washed with saturated NaCl (1×10 mL), dried over anhydrous Na₂SO₄, and filtered. After filtration, the filtrate was concentrated under reduced pressure to give a residue. The residue was purified by silica gel column chromatography, eluted with CH₂Cl₂/MeOH (10:01) to afford tert-butyl 4-[2-(7-fluoro-2-methylindazol-5-yl)-6-methylthieno [2,3-d][1,3]thiazol-5-yl]piperazine-1-carboxylate (20.0 mg, 26.05%) as a solid. LCMS (ES, m/z): 488 [M+H]⁺.

### Synthesis of Compound 194

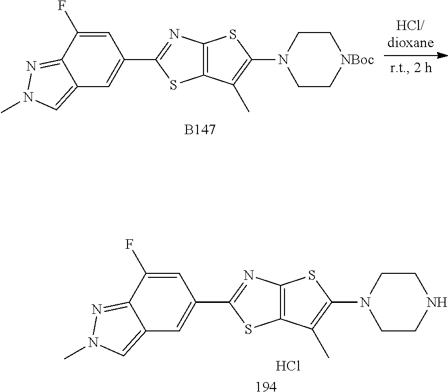

A mixture of tert-butyl 4-[2-(7-fluoro-2-methylindazol-5-yl)-6-methylthieno[2,3-d][1,3]thiazol-5-yl] piperazine-1-carboxylate (20.0 mg, 0.04 mmol, 1.00 equiv) and HCl (gas) in 1,4-dioxane (5 mL, 0.14 mmol, 3.34 equiv) was stirred for 2 h at room temperature. The resulting mixture was concentrated under reduced pressure to give a residue. The residue was purified by prep-HPLC (Condition 7, Gradient 6) to afford 7-fluoro-2-methyl-5-[6-methyl-5-(piperazin-1-yl)thieno[2,3-d] [1,3]thiazol-2-yl]indazole (2.20 mg, 13.8%) as a solid. LCMS (ES, m/z):388 [M+H]⁺. ¹H NMR (400 MHz, DMSO-d₆) δ 9.02 (s, 2H), 8.63 (d, J=2.7 Hz, 1H), 8.22 (d, J=1.3 Hz, 1H), 7.64 (dd, J=12.5, 1.4 Hz, 1H), 4.24 (s, 3H), 3.30 (s, 4H), 3.15 (t, J=5.0 Hz, 4H), 2.30 (s, 3H)

### Example 72: Synthesis of Compounds 166-173, 175, 176, 178, and 181

### Synthesis of Intermediate B148

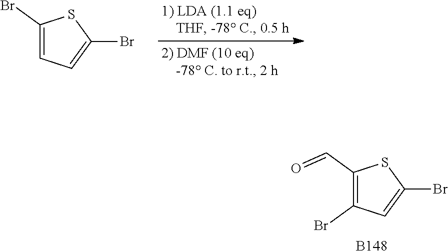 [SMILES: Brc1ccc(Br)s1 ; "B148" ; O=Cc1sc(Br)cc1Br]

To a stirred solution of 2,5-dibromothiophene (40 g, 165.337 mmol, 1.00 equiv) in THF (400 mL) was added LDA (19.48 g, 181.871 mmol, 1.1 equiv) dropwise at −78° C. under nitrogen atmosphere. The resulting mixture was stirred for 30 min at −78° C. under nitrogen atmosphere. To the reaction mixture was added DMF (127.95 mL, 1653.370 mmol, 10 equiv) dropwise over 5 min at −78° C. The resulting mixture was stirred for an additional 2 h at room temperature. The reaction mixture was quenched with water/ice (30 mL) at 0° C., then extracted with ethyl acetate (3×30 mL). The organic layers were combined, washed with brine (2×10 mL), dried over anhydrous Na₂SO₄, and filtered. After filtration, the filtrate was concentrated under reduced pressure to afford 3,5-dibromothiophene-2-carbaldehyde (44 g, 98.59%) as a solid. LCMS (ESI, m z): 269 [M+H]⁺.

### Synthesis of Intermediate B149

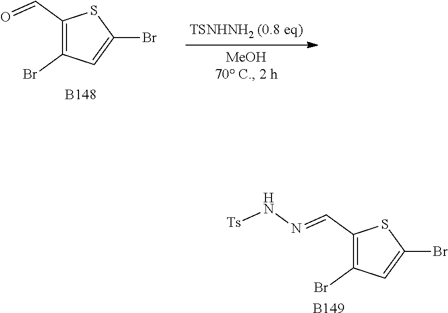

A mixture of 3,5-dibromothiophene-2-carbaldehyde (50 g, 185.226 mmol, 1.00 equiv), TsNHNH₂ (27.60 g, 148.181 mmol, 0.8 equiv), and methanol (500 mL) was stirred for 2 h at 70° C. under nitrogen atmosphere. The resulting mixture was concentrated under reduced pressure and a precipitate formed. The precipitated solid was collected by filtration, and washed with hexane (2×10 mL) to afford N-[(1E)-(3,5-dibromothiophen-2-yl)methylidene]-4-methylbenzene sulfonohydrazide (50 g, 61.61%) as a solid. LCMS (ESI, m z): 439 [M+H]⁺.

### Synthesis of Intermediate B150

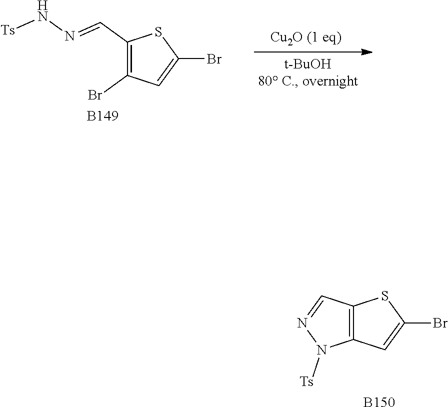 [SMILES: "B149" ; Cc1ccc(S(=O)(=O)N/N=C/c2sc(Br)cc2Br)cc1 ; "B150" ; Cc1ccc(S(=O)(=O)n2ncc3sc(Br)cc32)cc1]

A mixture of N-[(1E)-(3,5-dibromothiophen-2-yl)methylidene]-4-methylbenzenesulfonohydrazide (50 g, 114.116 mmol, 1.00 equiv), Cu₂O (16.33 g, 114.116 mmol, 1 equiv), and t-BuOH (500 mL) was stirred overnight at 80° C. under N₂ atmosphere. The resulting mixture was filtered, and the filter cake was washed with tert-butanol (1×10 mL). The filtrate was cooled to room temperature, and a precipitate formed that was collected by filtration. The filtrate was concentrated under reduced pressure to yield further precipitate, and the precipitated solid was collected by filtration to afford 5-bromo-1-(4-methylbenzenesulfonyl)thieno[3,2-c]pyrazole (33 g, 80.95%) as a solid. LCMS (ESI, m z): 358 [M+H]⁺.

### Synthesis of Intermediate B151

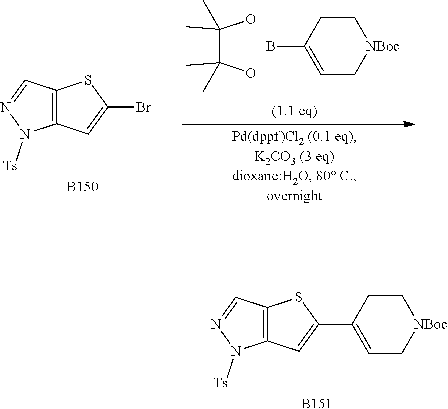

To a mixture of Pd(dppf)Cl₂ (2.05 g, 2.799 mmol, 0.1 equiv) and K₂CO₃ (11.61 g, 83.976 mmol, 3 equiv) in dioxane (100 mL) and water (20 mL) was added 5-bromo-1-(4-methylbenzenesulfonyl)thieno[3,2-c]pyrazole (10 g, 27.992 mmol, 1.00 equiv) and tert-butyl 4-(4,4,5,5-tetramethyl-1,3,2-dioxaborolan-2-yl)-3,6-dihydro-2H-pyridine-1-carboxylate (9.52 g, 30.791 mmol, 1.1 equiv). After stirring overnight at 80° C. under a nitrogen atmosphere, the resulting mixture was concentrated under reduced pressure to give a residue. The residue was purified by silica gel column chromatography, eluted with PE/EA (1:1) to afford tert-butyl 4-[1-(4-methylbenzenesulfonyl)thieno[3,2-c] pyrazol-5-yl]-3,6-dihydro-2H-pyridine-1-carboxylate (9.5 g, 73.85%) as a solid. LCMS (ESI, m z): 459 [M+H]⁺.

### Synthesis of Intermediate B152

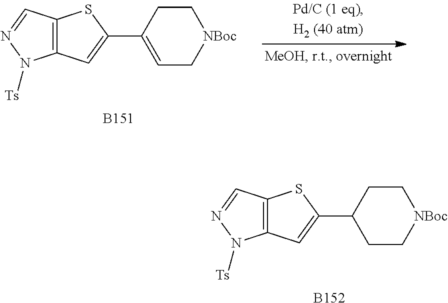

To a solution of tert-butyl 4-[1-(4-methylbenzenesulfonyl)thieno[3,2-c]pyrazol-5-yl]-3,6-dihydro-2H-pyridine-1-carboxylate (9.5 g, 20.671 mmol, 1.00 equiv) in methanol (100 mL) was added Pd/C (10%, 3 g) under nitrogen atmosphere in a 250 mL sealed tube. The reaction mixture was hydrogenated at room temperature for 8 days under hydrogen atmosphere using a hydrogen balloon, filtered through a Celite pad, and concentrated under reduced pressure to afford tert-butyl 4-[1-(4-methylbenzenesulfonyl)thieno[3,2-c]pyrazol-5-yl] piperidine-1-carboxylate (7.7 g, 80.70%) as a solid. LCMS (ESI, m/z): 461 [M+H]⁺.

### Synthesis of Intermediate B153

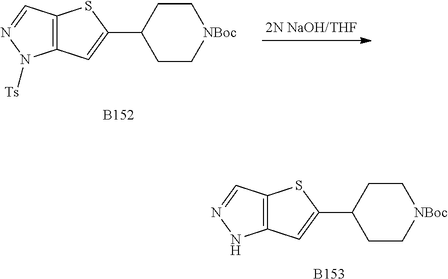 [SMILES: "B152" ; "B153" ; Cc1ccc(S(=O)(=O)n2ncc3sc(C4CCN(C(=O)OC(C)(C)C)CC4)cc32)cc1 ; CC(C)(C)OC(=O)N1CCC(c2cc3[nH]ncc3s2)CC1]

A mixture of tert-butyl 4-[1-(4-methylbenzenesulfonyl)thieno[3,2-c]pyrazol-5-yl]piperidine-1-carboxylate (7.7 g, 16.681 mmol, 1.00 equiv), NaOH(2M) (80 mL), THF (80 mL) was stirred overnight at 50° C. under nitrogen atmosphere. The resulting mixture was extracted with CH₂Cl₂ (3×80 mL). The organic layers were combined, washed with brine (2×80 mL), dried over anhydrous Na₂SO₄, and filtered. After filtration, the filtrate was concentrated under reduced pressure to give a residue. The residue was purified by reverse flash chromatography (Condition 2, Gradient 3) tert-butyl 4-{1H-thieno[3,2-c]pyrazol-5-yl}piperidine-1-carboxylate (3 g, 58.50%) as a solid. LCMS (ESI, m z): 308 [M+H]⁺.

### Synthesis of Intermediate B154

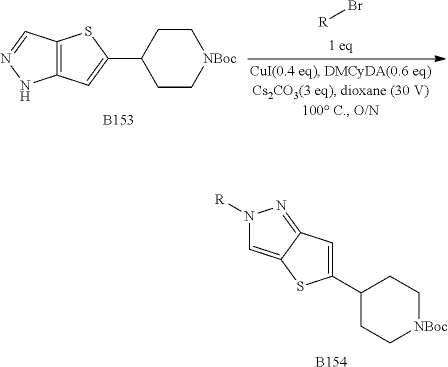

A mixture of tert-butyl 4-{1H-thieno[3,2-c]pyrazol-5-yl}piperidine-1-carboxylate (200 mg, 0.651 mmol, 1.00 equiv), 6-bromo-8-fluoro-2-methylimidazo[1,2-a]pyridine (149 mg, 0.651 mmol, 1 equiv), CuI (50 mg, 0.260 mmol, 0.4 equiv), (1R,2R)-1-N,2-N-dimethylcyclohexane-1,2-diamine (55 mg, 0.390 mmol, 0.60 equiv), Cs₂CO₃ (636 mg, 1.953 mmol, 3 equiv), and dioxane (20 mL) was stirred overnight at 100° C. under nitrogen atmosphere. The reaction mixture was cooled to room temperature, then diluted with water (20 mL). The resulting mixture was extracted with CH₂Cl₂ (3×20 mL). The organic layers were combined, washed with brine (2×20 mL), dried over anhydrous Na₂SO₄, and filtered. After filtration, the filtrate was concentrated under reduced pressure to give a residue. The residue was purified by silica gel column chromatography, eluted with CH₂Cl₂/MeOH (12:1), followed by reverse flash chromatography (Condition 2, Gradient 3) and prep-HPLC (Condition 2, Gradient 14) to afford tert-butyl 4-(2-{8-fluoro-2-methylimidazo[1,2-a]pyridin-6-yl}thieno [3,2-c]pyrazol-5-yl)piperidine-1-carboxylate (60 mg, 20.24%) and tert-butyl 4-(1-{8-fluoro-2-methylimidazo [1,2-a]pyridin-6-yl}thieno[3,2-c]pyrazol-5-yl)piperidine-1-carboxylate (50 mg, 16.87%) as solids. LCMS (ESI, m z): 456 [M+H]⁺.

### Synthesis of Compound 166

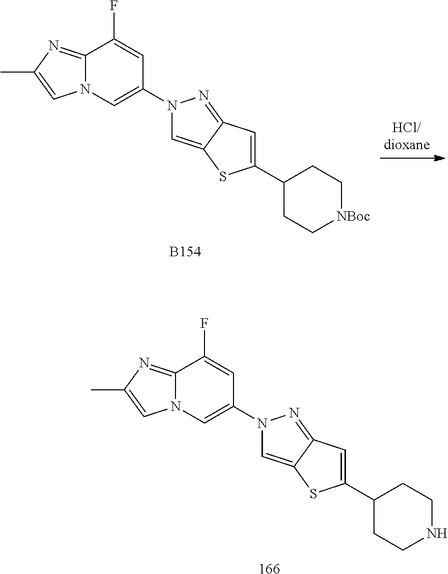 [SMILES: "B154" ; Cc1cn2cc(-n3cc4sc(C5CCN(C(=O)OC(C)(C)C)CC5)cc4n3)cc(F)c2n1 ; Cc1cn2cc(-n3cc4sc(C5CCNCC5)cc4n3)cc(F)c2n1 ; "166"]

A mixture of tert-butyl 4-(2-{8-fluoro-2-methylimidazo [1,2-a]pyridin-6-yl}thieno[3,2-c]pyrazol-5-yl)piperidine-1-carboxylate (30 mg, 0.066 mmol, 1.00 equiv) and HCl (gas) in 1,4-dioxane (2 mL) was stirred for 1 h at room temperature, then concentrated under reduced pressure to give a residue. The residue was dissolved in THF (2 mL) and purified by reverse flash chromatography (Condition 2, Gradient 14) to afford 4-(2-{8-fluoro-2-methylimidazo[1,2-a]pyridin-6-yl}thieno[3,2-c]pyrazol-5-yl)piperidine) (11.8 mg, 50.41%) as a solid.

Compounds 166-173, 175, 176, 178, and 181 were prepared according to the procedures described herein and outlined in this Example 72. The table below provides intermediates used in these procedures and final compound characterization data.

| | | LCMS | |
|---|---|---|---|
| | | (ESI, m/z) | |
| Compound No. and Structure | Coupling Reagent | [M + H]⁺ | ¹H NMR δ |
| 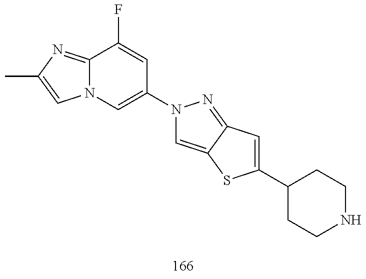 [SMILES: Cc1cn2cc(-n3cc4sc(C5CCNCC5)cc4n3)cc(F)c2n1] | 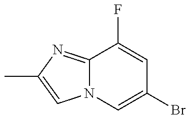 | 356 | (400 MHz, DMSO-d₆) δ 9.04 (s, 1H), 8.65 (s, 1H), 7.93 (d, J = 3.0 Hz, 1H), 7.82 (d, J = 12.1 Hz, 1H), 6.98 (s, 1H), 3.01 (d, J = 12.0 Hz, 2H), 2.92 (dd, J = 13.7, 9.6 Hz, 1H), 2.60 (d, J = 11.8 Hz, 2H), 2.38 (s, 3H), 1.91 (d, J = 12.6 Hz, 2H), 1.55 (qd, J = 12.2, 4.0 Hz, 2H) |
| 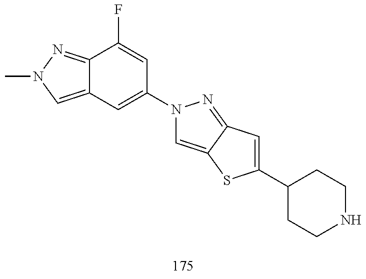 [SMILES: Cn1cc2cc(-n3cc4sc(C5CCNCC5)cc4n3)cc(F)c2n1] | 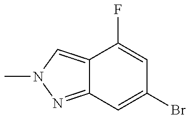 | 356 | (400 MHz, DMSO-d₆) δ 8.80 (s, 1H), 8.61 (s, 1H), 7.93 (s, 1H), 7.52 (d, J = 11.8 Hz, 1H), 6.99 (s, 1H), 4.20 (s, 3H), 3.06-2.98 (m, 2H), 2.92 (td, J = 12.0, 11.3, 5.5 Hz, 1H), 2.64-2.54 (m, 2H), 1.92 (d, J = 12.1 Hz, 2H), 1.55 (qd, J = 12.2, 4.0 Hz, 2H) |
| 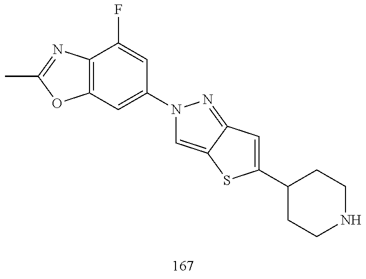 [SMILES: Cc1nc2c(F)cc(-n3cc4sc(C5CCNCC5)cc4n3)cc2o1] | 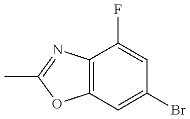 | 357 | (400 MHz, DMSO-d₆) δ 8.80 (s, 1H), 8.12 (s, 1H), 7.87 (d, J = 11.4 Hz, 1H), 6.99 (s, 1H), 3.02 (d, J = 12.6 Hz, 2H), 2.91 (td, J = 11.8, 5.8 Hz, 1H), 2.67 (s, 3H), 2.60 (d, J = 11.9 Hz, 2H), 1.95-1.87 (m, 2H), 1.55 (qd, J = 12.1, 3.9 Hz, 2H) |
| 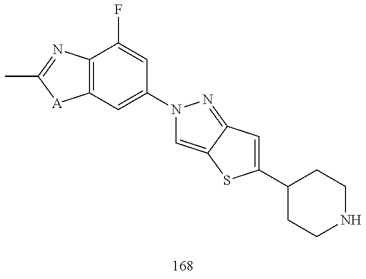 | 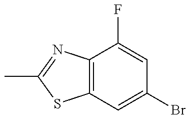 | 373 | (400 MHz, DMSO-d₆) δ 8.68 (d, J = 0.7 Hz, 1H), 8.39 (dd, J = 2.0, 0.8 Hz, 1H), 7.90 (dd, J = 12.0, 2.1 Hz, 1H), 6.95 (t, J = 0.9 Hz, 1H), 3.04-2.94 (m, 2H), 2.94 (ddq, J = 11.7, 7.6, 3.7 Hz, 1H), 2.85 (s, 3H), 2.69-2.58 (m, 2H), 1.99-1.90 (m, 2H), 1.60 (dtd, J = 12.7, 11.6, 4.0 Hz, 2H) |
| 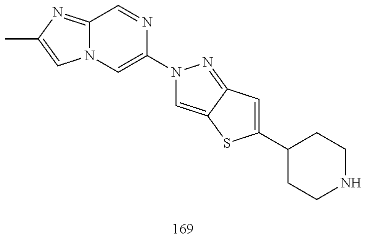 [SMILES: Cc1cn2cc(-n3cc4sc(C5CCNCC5)cc4n3)ncc2n1] | 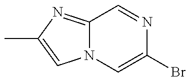 | 339 | (400 MHz, DMSO-d₆) δ 8.99 (d, J = 1.5 Hz, 1H), 8.95-8.90 (m, 1H), 8.02 (d, J = 17.8 Hz, 2H), 7.49 (s, 1H), 2.98 (dt, J = 11.4, 3.9 Hz, 2H), 2.64 (td, J = 12.2, 2.6 Hz, 2H), 2.46 (s, 3H), 1.96 (d, J = 12.5 Hz, 2H), 1.59 (qd, J = 12.0, 4.1 Hz, 2H) |
| 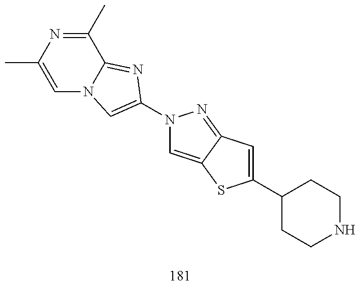 [SMILES: Cc1cn2cc(-n3cc4sc(C5CCNCC5)cc4n3)nc2c(C)n1] | 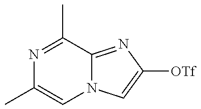 [SMILES: Cc1cn2cc(OS(=O)(=O)C(F)(F)F)nc2c(C)n1] | 353 | (400 MHz, DMSO-d₆) δ 8.62 (s, 1H), 8.28 (s, 1H), 8.22 (s, 1H), 6.94 (s, 1H), 3.04-2.96 (m, 2H), 2.92 (t, J = 11.9 Hz, 1H), 2.72 (s, 3H), 2.58 (td, J = 12.2, 2.3 Hz, 2H), 2.39 (s, 3H), 1.91 (d, J = 12.2 Hz, 2H), 1.57 (qd, J = 12.4, 3.9 Hz, 2H) |
| 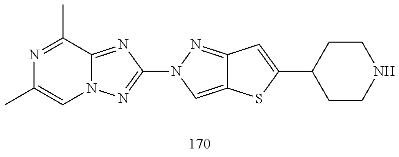 [SMILES: Cc1cn2nc(-n3cc4sc(C5CCNCC5)cc4n3)nc2c(C)n1] | 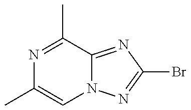 | 354 | (400 MHz, DMSO-d₆) δ 8.82 (s, 1H), 8.73 (s, 1H), 6.99 (s, 1H), 3.02 (dt, J = 12.3, 3.3 Hz, 2H), 2.92 (ddt, J = 11.6, 7.2, 3.6 Hz, 1H), 2.81 (s, 3H), 2.64- 2.54 (m, 2H), 2.49(s, 1H), 1.97-1.88 (m, 2H), 1.59 (dd, J = 12.2, 3.9 Hz, 1H), 1.53 (dd, J = 12.2, 3.9 Hz, |
| | | | 1H) |
| 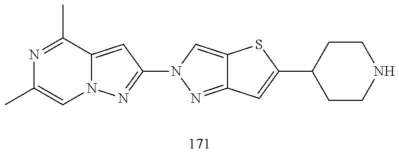 [SMILES: Cc1cn2nc(-n3cc4sc(C5CCNCC5)cc4n3)cc2c(C)n1] | 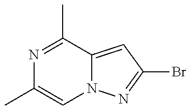 | 353 | (400 MHz, DMSO-d₆) δ 8.61 (s, 1H), 8.49 (d, J = 1.4 Hz, 1H), 7.17 (d, J = 1.0 Hz, 1H), 6.98 (s, 1H), 3.06-2.98 (m, 2H), 2.97- 2.86 (m, 1H), 2.70 (s, 3H), 2.59 (td, J = 12.1, 2.4 Hz, 2H), 2.43 (d, J = 1.0 Hz, 3H), 1.96-1.88 (m, 2H), 1.56 (qd, J = 12.2, |
| | | | 3.9 Hz, 2H) |
| 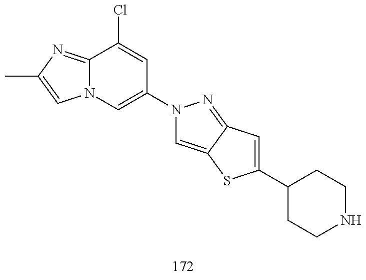 [SMILES: Cc1cn2cc(-n3cc4sc(C5CCNCC5)cc4n3)cc(Cl)c2n1] | 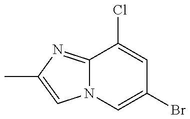 | 372 | (400 MHz, DMSO-d₆) δ 9.15 (d, J = 1.8 Hz, 1H), 8.68 (s, 1H), 8.05 (d, J = 1.8 Hz, 1H), 7.93 (s, 1H), 6.98 (s, 1H), 3.07-2.98 (m, 2H), 2.92 (t, J = 11.8 Hz, 1H), 2.64-2.56 (m, 2H), 2.39 (s, 3H), 1.95- 1.87 (m, 2H), 1.55 (qd, J = 12.2, 3.9 Hz, 2H) |
| 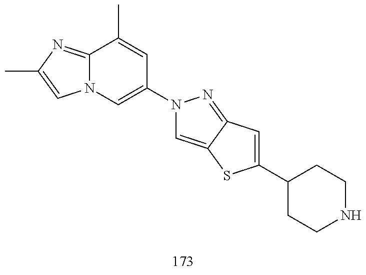 [SMILES: Cc1cn2cc(-n3cc4sc(C5CCNCC5)cc4n3)cc(C)c2n1] | 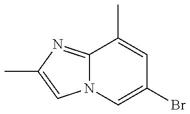 | 352 | (400 MHz, DMSO-d₆) δ 8.86 (d, J = 2.1 Hz, 1H), 8.51 (s, 1H), 7.74 (d, J = 1.0 Hz, 1H), 7.57 (dd, J = 2.2, 1.2 Hz, 1H), 6.93 (d, J = 1.0 Hz, 1H), 3.04 (m, 2H), 2.93 (ddt, J = 15.0, 7.8, 3.8 Hz, 1H), 2.63 (td, J = 11.9, 2.4 Hz, 2H), 2.57 (s, 3H), 2.38 (d, J = 0.8 Hz, 3H), 2.18 (s, 1H), 2.00- 1.89 (m, 2H), 1.67- 1.52 (m, 2H) |
| 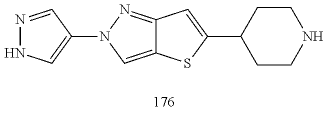 [SMILES: c1n[nH]cc1-n1cc2sc(C3CCNCC3)cc2n1] | 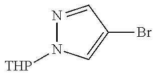 | 274 | (400 MHz, DMSO-d₆) δ 12.87 (s, 1H), 8.25 (s, 1H), 7.99 (s, 2H), 6.87 (s, 1H), 3.08-3.01 (m, 1H), 2.89 (tt, J = 11.6, 3.8 Hz, 1H), 2.61 (td, J = 12.1, 2.5 Hz, 2H), 1.96-1.86 (m, 2H), |
| | | | 1.56 (qd, J = 11.9, 4.0 Hz, |
| | | | 2H) |
| 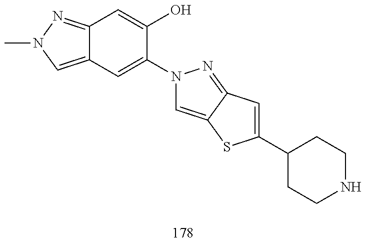 [SMILES: Cn1cc2cc(-n3cc4sc(C5CCNCC5)cc4n3)c(O)cc2n1] | 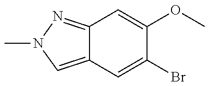 | 354 | (400 MHz, DMSO-d₆) δ 10.14 (s, 1H), 8.28 (s, 1H), 7.91 (s, 1H), 7.73 (s, 1H), 7.02 (s, 1H), 6.80 (s, 1H), 4.11 (s, 3H), 2.98 (dt, J = 12.6, 3.3 Hz, 2H), 2.89 (ddt, J = 11.4, 7.4, 3.8 Hz, 1H), 2.57 (dd, J = 12.2, 2.4 Hz, 2H), 1.88 (d, J = 12.3 Hz, 2H), 1.50 (qd, J = 12.1, 3.9 Hz, 2H) |

### Example 73: Synthesis of Compound 185

### Synthesis of Intermediate B155

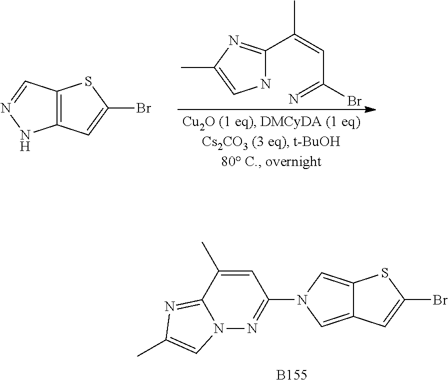

A mixture of 5-bromo-1H-thieno[3,2-c] pyrazole (2.50 g, 12.3 mmol, 1.00 equiv), 6-bromo-2,8-dimethylimidazo[1,2-b] pyridazine (2.78 g, 12.3 mmol, 1.00 equiv), Cu₂O (1.76 g, 12.3 mmol, 1.00 equiv), (1R,2R)-1-N,2-N-dimethylcyclohexane-1,2-diamine (1.75 g, 12.3 mmol, 1.00 equiv) and Cs₂CO₃ (12.03 g, 36.9 mmol, 3.00 equiv) in t-BuOH (50.0 mL) was stirred for 16 h at 80° C. under nitrogen atmosphere. The reaction mixture was cooled to 25° C., then diluted with water (50.0 mL), and extracted with ethyl acetate (2×50.0 mL). The organic layers were combined, dried over anhydrous Na₂SO₄, and filtered. After filtration, the filtrate was concentrated under reduced pressure to give a residue. The residue was purified by silica gel column chromatography, eluted with PE/EA (1:5), followed by Chiral-Prep-HPLC (Condition 2, Gradient 2) to afford 6-{5-bromothieno[3,2-c] pyrazol-2-yl}-2,8-dimethylimidazo[1,2-b] pyridazine (270.00 mg, 6.3%) as a solid. LCMS (ES, m/z): 348 [M+H]⁺.

### Synthesis of Intermediate B156

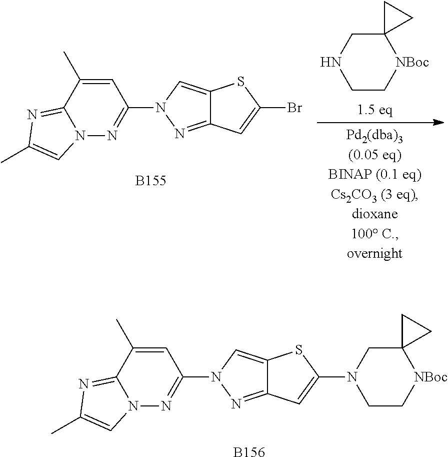 [SMILES: "B156" ; "B155" ; Cc1cn2nc(-n3cc4sc(Br)cc4n3)cc(C)c2n1 ; Cc1cn2nc(-n3cc4sc(N5CCN(C(=O)OC(C)(C)C)C6(CC6)C5)cc4n3)cc(C)c2n1 ; CC(C)(C)OC(=O)N1CCNCC12CC2]

A mixture of 6-{5-bromothieno[3,2-c] pyrazol-2-yl}-2,8-dimethylimidazo[1,2-b] pyridazine (40.00 mg, 0.1 mmol, 1.00 equiv), tert-butyl 4,7-diazaspiro [2.5] octane-4-carboxylate (36.58 mg, 0.1 mmol, 1.50 equiv), Pd₂(dba)₃ (5.26 mg, 0.006 mmol, 0.05 equiv), BINAP (7.15 mg, 0.012 mmol, 0.10 equiv) and Cs₂CO₃ (112.28 mg, 0.3 mmol, 3.00 equiv) in dioxane (0.8 mL) was stirred for 16 h at 100° C. under nitrogen atmosphere. The reaction mixture was cooled to 25° C., then diluted with water (50.0 mL), and extracted with ethyl acetate (2×50.0 mL). The organic layers were combined, dried over anhydrous Na₂SO₄, and filtered. After filtration, the filtrate was concentrated under reduced pressure to give a residue. The residue was purified by silica gel column chromatography, eluted with PE/EA (1:4) to afford tert-butyl 7-(2-{2,8-dimethylimidazo[1,2-b] pyridazin-6-yl} thieno [3,2-c] pyrazol-5-yl)-4,7-diazaspiro [2.5] octane-4-carboxylate (25.00 mg, 45.3%) as a solid. LCMS (ES, m/z): 480 [M+H]⁺.

### Synthesis of Compound 185

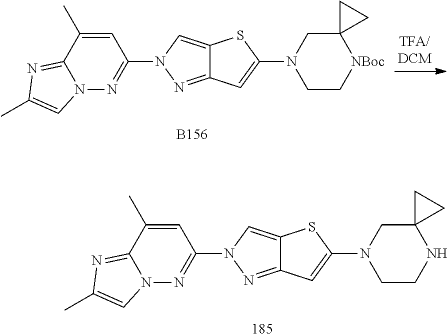 [SMILES: "B156" ; Cc1cn2nc(-n3cc4sc(N5CCN(C(=O)OC(C)(C)C)C6(CC6)C5)cc4n3)cc(C)c2n1 ; Cc1cn2nc(-n3cc4sc(N5CCNC6(CC6)C5)cc4n3)cc(C)c2n1 ; "185"]

A solution of tert-butyl 7-(2-{2,8-dimethylimidazo[1,2-b] pyridazin-6-yl} thieno [3,2-c]pyrazol-5-yl)-4,7-diazaspiro [2.5] octane-4-carboxylate (25.00 mg, 0.05 mmol, 1.00 equiv) in TFA (0.2 mL) and DCM (0.4 mL) was stirred for 1 h at room temperature. The resulting mixture was concentrated under vacuum to give a residue. The residue was purified by Chiral-Prep-HPLC (Condition 5, Gradient 19) to afford 7-(2-{2,8-dimethylimidazo[1,2-b]pyridazin-6-yl}thieno [3,2-c] pyrazol-5-yl)-4,7-diazaspiro [2.5] octane (2.20 mg, 10.7%) as a solid. LCMS (ES, m/z): 380 [M+H]⁺. ¹H NMR (400 MHz, DMSO-d₆) δ 8.53 (s, 1H), 7.96 (d, J=1.0 Hz, 1H), 7.69 (d, J=1.2 Hz, 1H), 5.97 (s, 1H), 3.21 (d, J=5.2 Hz, 2H), 3.09 (s, 2H), 2.89 (s, 2H), 2.61 (d, J=1.1 Hz, 3H), 2.39 (d, J=0.8 Hz, 3H), 0.54 (d, J=15.1 Hz, 4H).

### Example 74: Synthesis of Compound 188

### Synthesis of Compound 188

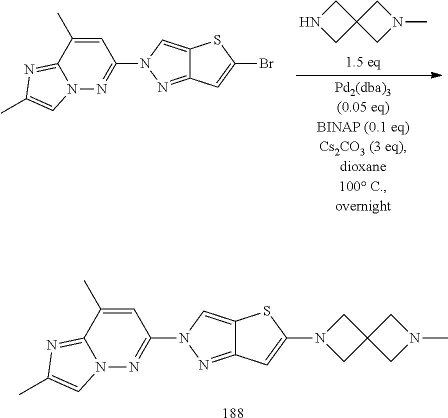

A mixture of 6-{5-bromothieno[3,2-c] pyrazol-2-yl}-2,8-dimethylimidazo [1,2-b] pyridazine (25.00 mg, 0.07 mmol, 1.00 equiv), 2-methyl-2,6-diazaspiro [3.3] heptane (9.66 mg, 0.08 mmol, 1.20 equiv), Pd₂(dba)₃ (3.29 mg, 0.004 mmol, 0.05 equiv), BINAP (4.47 mg, 0.007 mmol, 0.10 equiv), and Cs₂CO₃ (70.18 mg, 0.2 mmol, 3.00 equiv) in dioxane (0.8 mL) was stirred for 16 h at 100° C. under nitrogen atmosphere. The reaction mixture was cooled to 25° C. The resulting mixture was diluted with water (50.0 mL) and extracted with ethyl acetate (2×50.0 mL). The organic layers were combined, dried over anhydrous Na₂SO₄, and filtered. After filtration, the filtrate was concentrated under reduced pressure to give a residue. The residue was purified by silica gel column chromatography, eluted with PE/EA (1:4), followed by Chiral-Prep-HPLC (Condition 2, Gradient 3) to afford 2-(2-{2,8-dimethylimidazo[1,2-b]pyridazin-6-yl} thieno [3,2-c] pyrazol-5-yl)-6-methyl-2,6-diazaspiro [3.3] heptane (5.40 mg, 19.7%) as a solid. LCMS (ES, m/z): 380 [M+H]⁺. ¹H NMR (400 MHz, DMSO-d₆) δ 8.53 (s, 1H), 7.97 (d, J=1.0 Hz, 1H), 7.69 (d, J=1.3 Hz, 1H), 5.71 (s, 1H), 4.03 (s, 4H), 3.29 (s, 4H), 2.61 (d, J=1.2 Hz, 3H), 2.39 (s, 3H), 2.18 (s, 3H).

### Example 75: Synthesis of Compound 189

### Synthesis of Compound 189

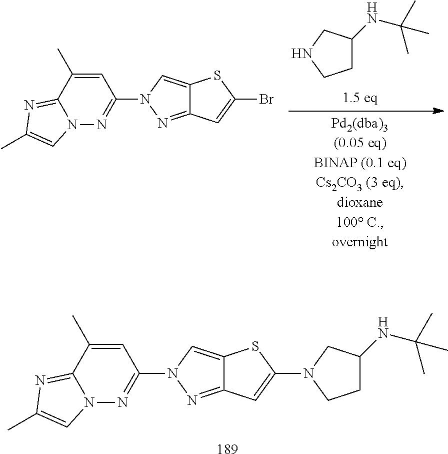

A mixture of 6-{5-bromothieno[3,2-c] pyrazol-2-yl}-2,8-dimethylimidazo[1,2-b] pyridazine (40.00 mg, 0.1 mmol, 1.00 equiv), N-tert-butylpyrrolidin-3-amine (24.51 mg, 0.1 mmol, 1.50 equiv), Pd₂(dba)₃ (5.26 mg, 0.006 mmol, 0.05 equiv), BINAP (7.15 mg, 0.01 mmol, 0.10 equiv) and Cs₂CO₃ (112.28 mg, 0.3 mmol, 3.00 equiv) in dioxane (1.6 mL) was stirred for 16 h at 100° C. under nitrogen atmosphere. The reaction mixture was cooled to 25° C., then diluted with water (50.0 mL) and extracted with ethyl acetate (2×50.0 mL). The organic layers were combined, dried over anhydrous Na₂SO₄, and filtered. After filtration, the filtrate was concentrated under reduced pressure to give a residue. The residue was purified by silica gel column chromatography, eluted with PE/EA (1:4), followed by Prep-HPLC (Condition 5, Gradient 20) to afford N-tert-butyl-1-(2-{2,8-dimethylimidazo[1,2-b] pyridazin-6-yl}thieno[3,2-c] pyrazol-5-yl) pyrrolidin-3-amine (6.80 mg, 14.2%) as a solid. LCMS (ES, m/z): 410 [M+H]⁺. ¹H NMR (400 MHz, DMSO-d₆) δ 8.49 (s, 1H), 7.95 (d, J=1.0 Hz, 1H), 7.68 (d, J=1.3 Hz, 1H), 5.54 (s, 1H), 3.53 (d, J=8.3 Hz, 2H), 3.42 (s, 1H), 3.30 (s, 1H), 2.98 (s, 1H), 2.60 (d, J=1.1 Hz, 3H), 2.39 (s, 3H), 2.18 (s, 1H), 1.77 (s, 1H), 1.07 (s, 9H).

### Example 76: Synthesis of Compound 246

### Synthesis of Intermediate B157

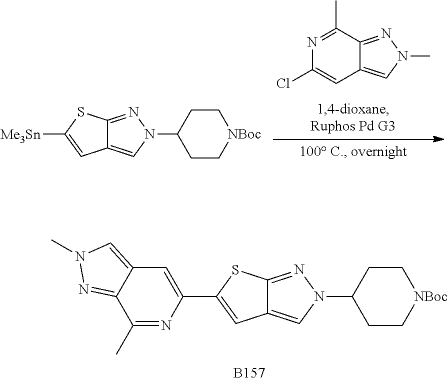

A mixture of tert-butyl 4-[5-(trimethylstannyl)thieno[2,3-c]pyrazol-2-yl]piperidine-1-carboxylate (160 mg, 0.340 mmol, 1.00 equiv), 5-chloro-2,7-dimethylpyrazolo[3,4-c]pyridine (67.98 mg, 0.374 mmol, 1.1 equiv) and RuPhos Palladacycle Gen.3 (28.46 mg, 0.034 mmol, 0.1 equiv) in 1,4-dioxane (3 mL) was stirred overnight at 100° C. under nitrogen atmosphere. The resulting mixture was concentrated under reduced pressure to give a residue. The residue was purified by reverse flash chromatography (Condition 1, Gradient 2) to afford tert-butyl 4-(5-{2,7-dimethylpyrazolo[3,4-c]pyridin-5-yl} thieno[2,3-c] pyrazol-2-yl) piperidin-1-carboxylate (65 mg, 29.55%) as a solid. LCMS (ESI, m z): 453[M+H]⁺.

### Synthesis of Compound 246

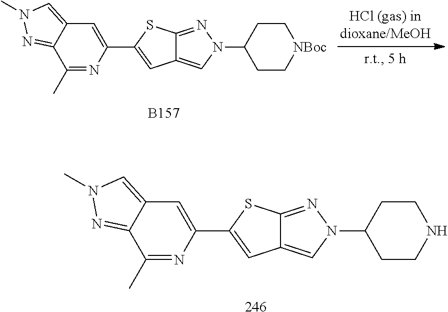 [SMILES: "B157" ; Cc1nc(-c2cc3cn(C4CCN(C(=O)OC(C)(C)C)CC4)nc3s2)cc2cn(C)nc12 ; Cc1nc(-c2cc3cn(C4CCNCC4)nc3s2)cc2cn(C)nc12 ; "246"]

To a stirred solution of tert-butyl 4-(5-{2,7-dimethylpyrazolo[3,4-c]pyridin-5-yl}thieno[2,3-c] pyrazol-2-yl)piperidine-1-carboxylate (65 mg, 0.144 mmol, 1 equiv) in methanol (3.25 mL) was added HCl (gas) in 1,4-dioxane (3.25 mL) dropwise at room temperature under air atmosphere. The resulting mixture was stirred for 5 h at room temperature under air atmosphere, then concentrated under vacuum to give a residue. The residue was purified by Prep-HPLC (Condition 1, Gradient 4) to afford 4-(5-{2,7-dimethylpyrazolo[3,4-c]pyridin-5-yl}thieno[2,3-c]pyrazol-2-yl)piperidine hydrochloride (12.1 mg, 21.36%) as a solid. LCMS (ESI, m/z): 353[M+H]. ¹H NMR (400 MHz, DMSO-d₆): δ 9.28 (s, 1H), 9.08 (s, 1H), 8.45 (s, 1H), 8.07 (s, 1H), 7.96 (s, 1H), 7.64 (s, 1H), 4.69 (td, J=9.3, 4.7 Hz, 1H), 4.28 (s, 3H), 3.43 (d, J=12.7 Hz, 2H), 3.12 (q, J=12.5, 12.0 Hz, 2H), 2.84 (s, 3H), 2.34 (td, J=9.8, 4.2 Hz, 4H).

### Example 77: Synthesis of Compounds 244 and 245

### Synthesis of Intermediate B158

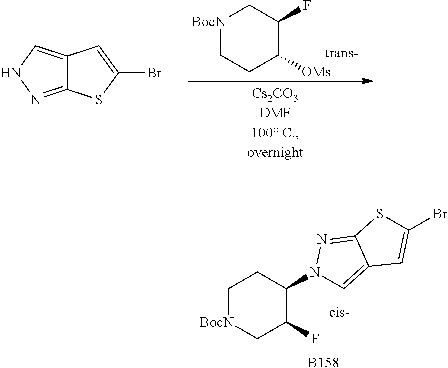 [SMILES: "B158" ; Brc1cc2c[nH]nc2s1 ; CC(C)(C)OC(=O)N1CC[C@@H](OS(C)(=O)=O)[C@H](F)C1 ; CC(C)(C)OC(=O)N1CC[C@@H](n2cc3cc(Br)sc3n2)[C@@H](F)C1]

To a stirred mixture of 5-bromo-2H-thieno[2,3-c]pyrazole (400 mg, 1.970 mmol, 1 equiv) and tert-butyl (3R,4R)-3-fluoro-4-(methanesulfonyloxy)piperidine-1-carboxylate (702.86 mg, 2.364 mmol, 1.2 equiv) in DMF (8 mL) was added Cs₂CO₃ (1925.46 mg, 5.910 mmol, 3 equiv) in portions at room temperature under nitrogen atmosphere. The reaction mixture was stirred at 100° C. overnight, then concentrated under vacuum to give a residue. The residue was purified by silica gel column chromatography, eluted with PE/EA (1:1) to afford tert-butyl (3S,4R)-4-{5-bromothieno[2,3-c]pyrazol-2-yl}-3-fluoropiperidine-1-carboxylate (380 mg, 47.71%) as a solid.

### Synthesis of Intermediates B159 and B160

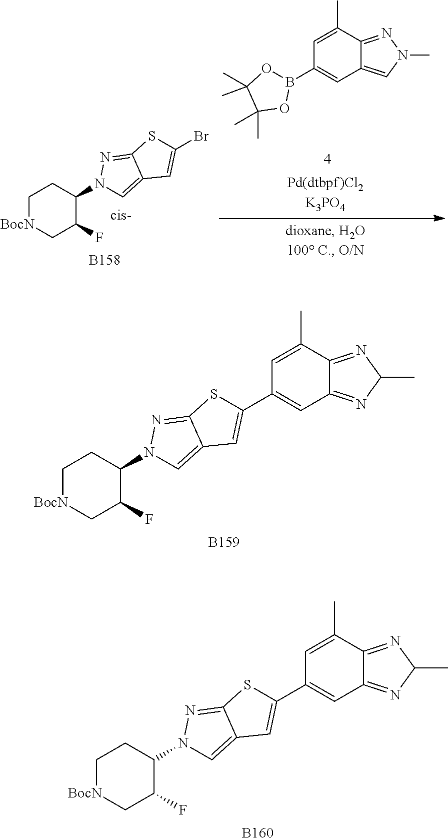

To a stirred mixture of tert-butyl (3S,4R)-4-{5-bromothieno[2,3-c]pyrazol-2-yl}-3-fluoropiperidine-1-carboxylate (350 mg, 0.866 mmol, 1 equiv) and 2,7-dimethyl-5-(4,4,5,5-tetramethyl-1,3,2-dioxaborolan-2-yl)indazole (282.73 mg, 1.039 mmol, 1.2 equiv) in dioxane (3.50 mL) and water (0.70 mL) was added Pd(dtbpf)Cl₂ (56.42 mg, 0.087 mmol, 0.1 equiv) and K₃PO₄ (551.27 mg, 2.598 mmol, 3 equiv) at room temperature under nitrogen atmosphere. The reaction mixture was stirred at 100° C. for 2 h. The resulting mixture was concentrated under vacuum to give a residue. The residue was purified by silica gel column chromatography, eluted with PE/EA (1:1) to afford tert-butyl (3S,4R)-4-[5-(2,7-dimethylindazol-5-yl)thieno[2,3-c]pyrazol-2-yl]-3-fluoropiperidine-1-carboxylate (250 mg, 61.50%) as a solid. The residue was purified by prep-chiral-HPLC (Condition 4, Gradient 1) to afford tert-butyl (3S,4R)-4-[5-(2,7-dimethylindazol-5-yl)thieno [2,3-c]pyrazol-2-yl]-3-fluoropiperidine-1-carboxylate (15 mg, 6.00%) and tert-butyl (3R,4S)-4-[5-(2,7-dimethylindazol-5-yl)thieno[2,3-c]pyrazol-2-yl]-3-fluoropiperidine-1-carboxylate (10 mg, 4.00%) as solids.

### Synthesis of Compound 244

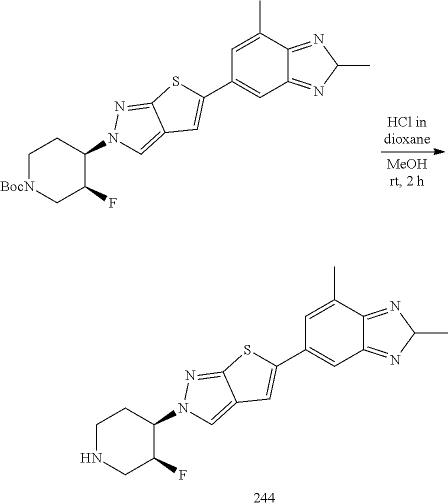 [SMILES: Cc1cc(-c2cc3cn([C@@H]4CCN(C(=O)OC(C)(C)C)C[C@@H]4F)nc3s2)cc2c1=NC(C)N=2 ; Cc1cc(-c2cc3cn([C@@H]4CCNC[C@@H]4F)nc3s2)cc2c1=NC(C)N=2 ; "244"]

To a stirred mixture of methane; tert-butyl (3S,4R)-4-[5-(2,7-dimethylindazol-5-yl)thieno [2,3-c]pyrazol-2-yl]-3-fluoropiperidine-1-carboxylate (15 mg, 0.031 mmol, 1 equiv) in methanol (0.5 mL) was added HCl (gas) in 1,4-dioxane (0.5 mL) at room temperature under nitrogen atmosphere. The reaction mixture was stirred at 25° C. for 2 h, then concentrated under vacuum to give a residue. The residue was purified by prep-HPLC (Condition 2, Gradient 3) to afford 5-{2-[(3S,4R)-3-fluoropiperidin-4-yl]thieno[2,3-c]pyrazol-5-yl}-2,7-dimethylindazole (6.8 mg, 59.59%) as a solid. LCMS (ES, m/z): 370 [M+H]⁺. ¹H NMR (400 MHz, DMSO-d₆) δ 8.33 (s, 1H), 8.03 (s, 1H), 7.72 (s, 1H), 7.42 (d, J=1.6 Hz, 1H), 7.38 (s, 1H), 4.94 (d, J=50.4 Hz, 1H), 4.66 (dd, J=31.8, 14.0 Hz, 1H), 4.17 (s, 3H), 3.19 (s, 1H), 3.10 (d, J=13.6 Hz, 1H), 2.90 (d, J=14.3 Hz, 1H), 2.80 (d, J=14.4 Hz, 1H), 2.54 (s, 3H), 2.23 (dd, J=12.4, 4.2 Hz, 1H), 1.96 (d, J=11.9 Hz, 1H).

### Synthesis of Compound 245

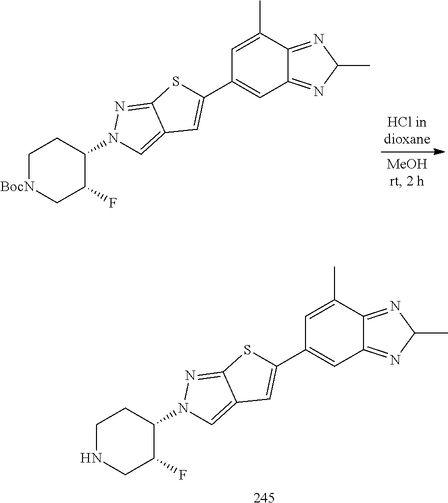 [SMILES: Cc1cc(-c2cc3cn([C@H]4CCN(C(=O)OC(C)(C)C)C[C@H]4F)nc3s2)cc2c1=NC(C)N=2 ; Cc1cc(-c2cc3cn([C@H]4CCNC[C@H]4F)nc3s2)cc2c1=NC(C)N=2 ; "245"]

To a stirred solution of tert-butyl (3R,4S)-4-[5-(2,7-dimethylindazol-5-yl)thieno[2,3-c]pyrazol-2-yl]-3-fluoropiperidine-1-carboxylate (10 mg, 0.021 mmol, 1 equiv) in methanol (0.5 mL) was added HCl (gas) in 1,4-dioxane (0.5 mL, 16.456 mmol, 772.74 equiv) at room temperature under nitrogen atmosphere. The reaction mixture was stirred at 25° C. for 2 h, then concentrated under vacuum to give a residue. The residue was purified by prep-HPLC (Condition 1, Gradient 3) to afford 5-{2-[(3R,4S)-3-fluoropiperidin-4-yl]thieno[2,3-c]pyrazol-5-yl}-2,7-dimethylindazole (7.9 mg, 100.41%) as a solid. LCMS (ES, m/z): 370 [M+H]⁺. ¹H NMR (400 MHz, DMSO-d₆) δ 8.33 (s, 1H), 8.03 (s, 1H), 7.72 (d, J=1.7 Hz, 1H), 7.42 (s, 1H), 7.38 (s, 1H), 4.93 (d, J=50.7 Hz, 1H), 4.65 (dd, J=30.8, 12.5 Hz, 1H), 4.17 (s, 3H), 3.19 (s, 1H), 3.10 (d, J=13.2 Hz, 1H), 2.90 (d, J=14.4 Hz, 1H), 2.80 (d, J=14.4 Hz, 1H), 2.54 (s, 3H), 2.22 (dt, J=13.1, 6.6 Hz, 1H), 1.96 (d, J=12.2 Hz, 1H).

### Example 78: Synthesis of Compound 212

### Synthesis of Intermediate B161

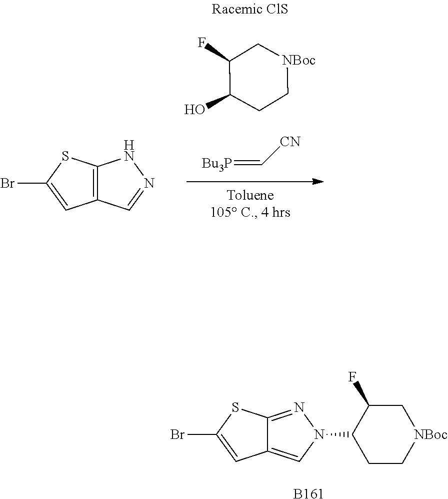

To a solution of 5-bromo-1H-thieno[2,3-c]pyrazole (150 mg, 739 umol) in toluene (15.0 mL) was added cis-tert-butyl 3-fluoro-4-hydroxypiperidine-1-carboxylate (240 mg, 1.09 mmol), followed by cyanomethylenetri-n-butylphosphorane (399 uL, 1.32 mmol) at room temperature under nitrogen atmosphere. The resulting mixture was stirred in refluxing toluene under nitrogen atmosphere for 2 h, then at room temperature overnight. Additional cyanomethylenetri-n-butylphosphorane (399 uL, 1.32 mmol) was added and the mixture refluxed for 2 hrs. Cis-tert-Butyl 3-fluoro-4-hydroxypiperidine-1-carboxylate (240 mg, 1.09 mmol) was added followed by cyanomethylenetri-n-butylphosphorane (399 uL, 1.32 mmol) and the mixture refluxed for an additional 4 hrs. The reaction mixture was allowed to cool to room temperature and concentrated under reduced pressure. The resulting residue was purified by silica gel column chromatography, eluted with Acetone/DCM to afford tert-butyl (1:1 mixture of 3S,4S and 3R,4R)-4-(5-bromo-2H-thieno[2,3-c]pyrazol-2-yl)-3-fluoropiperidine-1-carboxylate (151 mg, 84%) as a solid. LCMS (ES, m/z): 348 [M+H-t-Bu]⁺.

### Synthesis of Intermediate B162

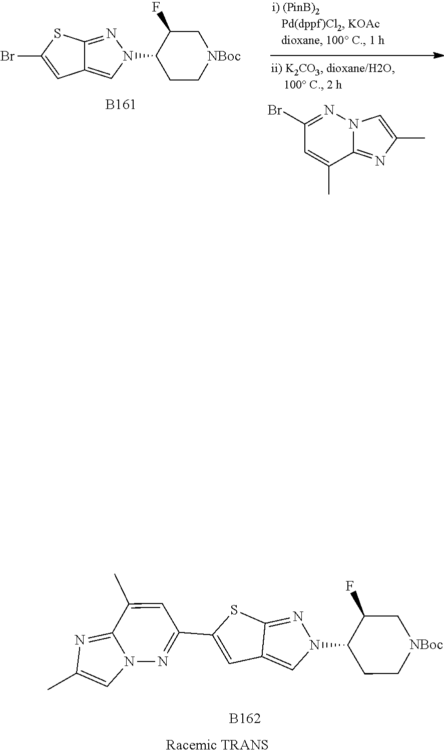

A mixture of 5-bromo-7-fluoro-2-methyl-2H-indazole (60 mg, 267 umol), bis(pinacolato)diboron (69 mg, 267 umol), 1,1 bis(diphenylphosphino)ferrocene dichloropalladium (II) (16.3 mg, 22.3 umol), and potassium acetate (55 mg, 557 umol) in 1,4-dioxane (1.4 mL) was heated to 115° C. for 2 h. To the reaction mixture was added a solution of potassium carbonate (92 mg, 0.67 mmol) in water (0.29 mL), followed by a solution of tert-butyl (1:1 mixture of 3S,4S and 3R,4R)-4-(5-(2,8-dimethylimidazo[1,2-b]pyridazin-6-yl)-2H-thieno[2,3-c]pyrazol-2-yl)-3-fluoropiperidine-1-carboxylate (90 mg, 223 umol) in dioxane (1.1 mL) under argon. The reaction mixture was heated at 90° C. for 1 h and then cooled to room temperature. The reaction mixture was filtered over Celite using 20% methanol in CH₂Cl₂ as eluent. The solvents were evaporated under reduced pressure to give a residue. The residue was purified by column chromatography on silica gel using a gradient of 0-100% ethyl acetate in hexanes to afford tert-butyl (1:1 mixture of 3S,4S and 3R,4R)-4-(5-(2,8-dimethylimidazo[1,2-b]pyridazin-6-yl)-2H-thieno[2,3-c]pyrazol-2-yl)-3-fluoropiperidine-1-carboxylate (33.0 mg, 32%) as a solid. LCMS (ES, m/z): 471.2 [M+H]⁺.

### Synthesis of Compound 212

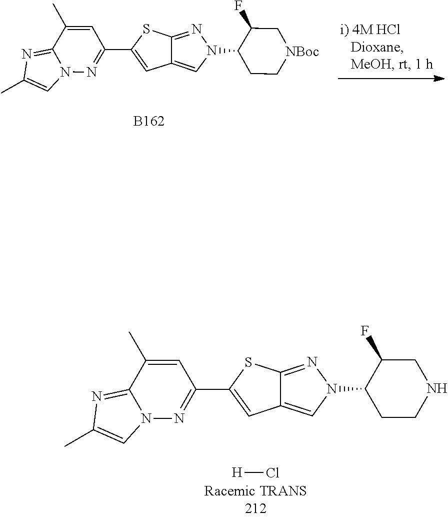 [SMILES: "B162" ; Cc1cn2nc(-c3cc4cn([C@H]5CCN(C(=O)OC(C)(C)C)C[C@@H]5F)nc4s3)cc(C)c2n1 ; Cc1cn2nc(-c3cc4cn([C@H]5CCNC[C@@H]5F)nc4s3)cc(C)c2n1 ; Cl]

To a suspension of tert-butyl (1:1 mixture of 3S,4S and 3R,4R)-4-(5-(2,8-dimethylimidazo[1,2-b]pyridazin-6-yl)-2H-thieno[2,3-c]pyrazol-2-yl)-3-fluoropiperidine-1-carboxylate (30.0 mg, 64 μmol) in methanol (1.2 mL) was added 4 M HCl in dioxane (1.0 mL, 4.0 mmol). The reaction mixture was stirred at room temperature for 2 h, and the volatiles removed under reduced pressure to give a residue. The resulting residue was suspended and triturated in ethyl acetate (5 mL), the solid was collected by vacuum filtration, washed with ethyl acetate (5 mL), and dried under vacuum. The solid was dissolved in a mixture of acetonitrile and water, then lyophilized to afford 5-(2,8-dimethylimidazo[1,2-b]pyridazin-6-yl)-2-((1:1 mixture of 3S,4S and 3R,4R)-3-fluoropiperidin-4-yl)-2H-thieno[2,3-c]pyrazole as an HCl salt (20.0 mg, 77%). LCMS (ES, m/z): 371.1 [M+H]⁺. ¹H NMR (CD₃OD, 400 MHz): δ_{H} 8.22 (2H, s), 8.19 (1H, s), 8.01 (1H, s), 5.32-5.16 (1H, m), 4.90-5.00 (1H, m), 3.97-3.87 (1H, m), 3.76-3.38 (3H, m), 2.73 (3H, s), 2.61 (3H, s), 2.55-2.48 (2H, m).

### Example 79: Synthesis of Compound 148

### Synthesis of Intermediate B163

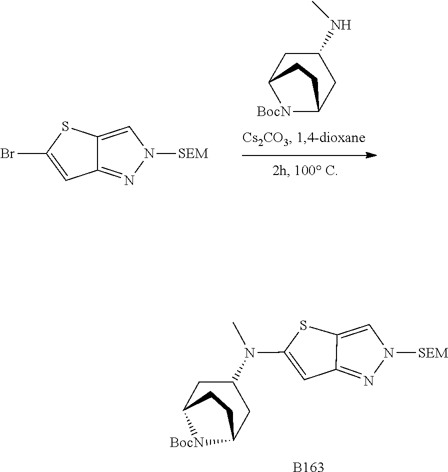

A mixture of 5-bromo-2-[[2-(trimethylsilyl)ethoxy]methyl]thieno[3,2-c]pyrazole (1.1 g, 3.30 mmol, 1.0 equiv), tert-butyl (1R,3R,5S)-3-(methylamino)-8-azabicyclo[3.2.1]octane-8-carboxylate (0.95 g, 0.004 mmol, 1.2 equiv), Pd-PEPPSI-IPentCl 2-methylpyridine (o-picoline) (0.28 g, 0.10 equiv), Cs₂CO₃ (3.23 g, 0.01 mmol, 3.0 equiv), and 1,4-dioxane (11.0 mL, 129.84 mmol, 39.35 equiv) was stirred for 2 h at 100° C. The resulting mixture was concentrated to give a residue. The residue was purified by silica gel column chromatography, eluted with ethyl acetate/petroleum ether (1:1) to afford tert-butyl (1R,3R,5S)-3-[methyl(2-[[2-(trimethylsilyl)ethoxy]methyl]thieno [3,2-c]pyrazol-5-yl)amino]-8-azabicyclo[3.2.1]octane-8-carboxylate (1 g, 61.5%) as a solid. LCMS (ES, m/z): 493 [M+H]⁺.

### Synthesis of Intermediate B164

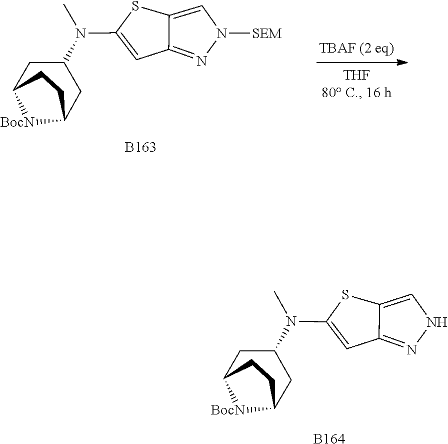

A mixture of tert-butyl (1R,3R,5S)-3-[methyl(2-[[2-(trimethylsilyl)ethoxy]methyl]thieno[3,2-c]pyrazol-5-yl)amino]-8-azabicyclo[3.2.1]octane-8-carboxylate (500.0 mg, 1.015 mmol, 1.0 equiv), TBAF (530.62 mg, 2.02 mmol, 2.0 equiv), and THE (10.0 mL, 123.43 mmol, 121.64 equiv) was stirred for 16 h at 80° C. The reaction mixture was then quenched with water/ice (10 mL). The resulting solution was extracted with ethyl acetate (3×10 mL) and the organic layers combined and concentrated to give a residue. The residue was purified by silica gel column chromatography with ethyl acetate/petroleum ether (1:1) to afford tert-butyl (1R,3R,5S)-3-[methyl(2H-thieno[3,2-c]pyrazol-5-yl)amino]-8-azabicyclo [3.2.1]octane-8-carboxylate (250 mg, 67.9%) as a solid. LCMS (ES, m/z): 363 [M+H]⁺.

### Synthesis of Intermediate B165

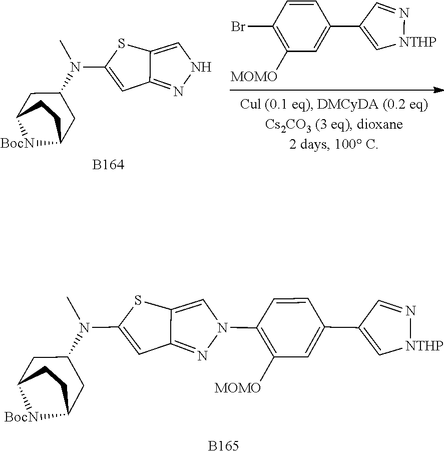

A mixture of tert-butyl (1R,3R,5S)-3-[methyl(2H-thieno[3,2-c]pyrazol-5-yl)amino]-8-azabicyclo[3.2.1]octane-8-carboxylate (220.0 mg, 0.61 mmol, 1.0 equiv), 4-[4-bromo-3-(methoxymethoxy)phenyl]-1-(oxan-2-yl)pyrazole (267.46 mg, 1.2 equiv), (1R,2S)—N1,N2-dimethylcyclohexane-1,2-diamine (17.27 mg, 0.12 mmol, 0.2 equiv), CuI (11.56 mg, 0.061 mmol, 0.1 equiv), Cs₂CO₃ (593.23 mg, 1.82 mmol, 3.0 equiv), and 1,4-dioxane (4.0 mL, 47.216 mmol, 77.8 equiv) was stirred for 2 days at 100° C. The reaction mixture was cooled to 25° C. and filtered to remove solids. The filtrate was concentrated under reduced pressure to give a residue. The residue was purified by Prep-HPLC Condition 2, Gradient 15) tert-butyl (1R,3R,5S)-3-([2-[2-(methoxymethoxy)-4-[1-(oxan-2-yl)pyrazol-4-yl]phenyl]thieno[3,2-c]pyrazol-5-yl](methyl)amino)-8-azabicyclo[3.2.1]octane-8-carboxylate (60 mg, 15.2%) and tert-butyl (1R,3R,5S)-3-([1-[2-(methoxymethoxy)-4-[1-(oxan-2-yl)pyrazol-4-yl]phenyl]thieno[3,2-c]pyrazol-5-yl](methyl)amino)-8-azabicyclo[3.2.1]octane-8-carboxylate (35 mg, 8.89%) as a solid. LCMS (ES, m/z): 649 [M+H]⁺.

### Synthesis of Compound 148

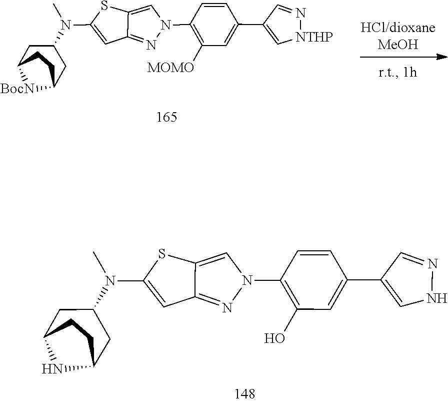

To a solution of tert-butyl (1R,3S,5S)-3-([2-[2-(methoxymethoxy)-4-[1-(oxan-2-yl)pyrazol-4-yl]phenyl]thieno[3,2-c]pyrazol-5-yl](methyl)amino)-8-azabicyclo[3.2.1]octane-8-carboxylate (60.0 mg) in methanol (1.0 mL) was added HCl (g) in 1,4-dioxane (1.0 mL) at 25° C. The resulting solution was stirred for 1 h at 25° C. The resulting mixture was concentrated to give a residue. The residue was purified by Prep-HPLC (Condition 2, Gradient 4) to afford 2-[5-[(1R,3R,5S)-8-azabicyclo[3.2.1]octan-3-yl(methyl)amino]thieno[3,2-c]pyrazol-2-yl]-5-(1H-pyrazol-4-yl)phenol (11.9 mg) as a solid. LCMS (ES, m/z): 421 [M+H]⁺. ¹H NMR (400 MHz, DMSO-d₆) δ 8.48 (s, 1H), 8.03 (s, 2H), 7.65 (d, J=8.4 Hz, 1H), 7.24 (d, J=2.0 Hz, 1H), 7.17 (dd, J=8.4, 1.9 Hz, 1H), 5.84 (s, 1H), 3.73 (t, J=11.4, 5.3 Hz, 1H), 3.51 (s, 2H), 2.80 (s, 3H), 1.51-1.86 (m, 8H).

### Example 80: Synthesis of Compounds 208, 210, 211, 214, 216, 218, 219, 221-226, 228, and 248

### Synthesis of Intermediate B166

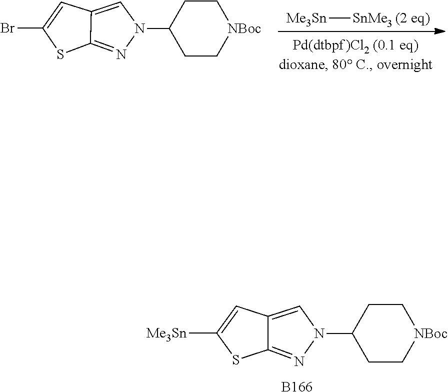

A mixture of tert-butyl 4-{5-bromothieno[2,3-c]pyrazol-2-yl}piperidine-1-carboxylate (500 mg, 1.294 mmol, 1.00 equiv), hexamethyldistannane (848.10 mg, 2.588 mmol, 2 equiv), and Pd(DtBPF)Cl₂ (84.36 mg, 0.129 mmol, 0.1 equiv) in 1,4-dioxane (10 mL, 113.471 mmol, 87.69 equiv) was stirred overnight at 80° C. under nitrogen atmosphere. The reaction mixture was cooled to room temperature, then quenched with saturated KF (aq.) (30 mL) at 0° C. and extracted with ethyl acetate (3×30 mL). The organic layers were combined, washed with brine (2×20 mL), dried over anhydrous Na₂SO₄, and filtered. After filtration, the filtrate was concentrated under reduced pressure to afford tert-butyl 4-[5-(trimethylstannyl)thieno[2,3-c]pyrazol-2-yl]piperidine-1-carboxylate (950 mg, 78.05%).

### Synthesis of Intermediate B167

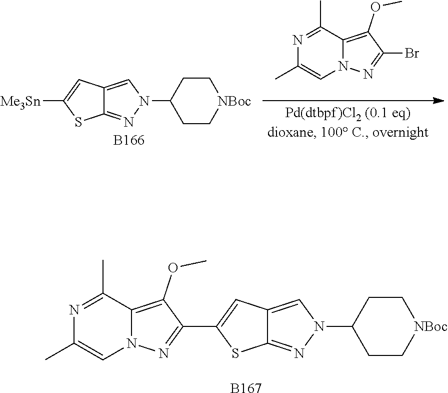

A mixture of 2-bromo-3-methoxy-4,6-dimethylpyrazolo[1,5-a]pyrazine (100 mg, 0.390 mmol, 1.00 equiv), tert-butyl 4-[5-(trimethylstannyl)thieno[2,3-c]pyrazol-2-yl]piperidine-1-carboxylate (201.97 mg, 0.429 mmol, 1.1 equiv), and Pd(DtBPF)Cl₂ (25.45 mg, 0.039 mmol, 0.1 equiv) in 1,4-dioxane (5 mL, 56.750 mmol, 145.34 equiv) was stirred overnight at 100° C. under nitrogen atmosphere. The resulting mixture was concentrated under reduced pressure to give a residue. The residue was purified by silica gel column chromatography, eluted with DCM/EA (2:1) to afford tert-butyl 4-(5-{3-methoxy-4,6-dimethylpyrazolo[1,5-a]pyrazin-2-yl}thieno [2,3-c]pyrazol-2-yl)piperidine-1-carboxylate (125 mg, 66.33%) as a solid.

### Synthesis of Compound 219

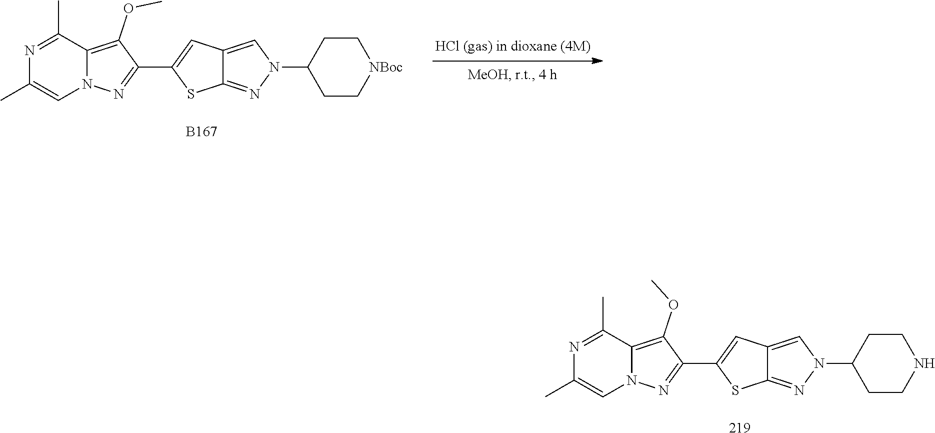

To a stirred solution of tert-butyl 4-(5-{3-methoxy-4,6-dimethylpyrazolo[1,5-a]pyrazin-2-yl}thieno[2,3-c]pyrazol-2-yl)piperidine-1-carboxylate (50 mg, 0.104 mmol, 1.00 equiv) in methanol (1.25 mL) was added HCl (gas) in 1,4-dioxane (1.25 mL) dropwise at room temperature under air atmosphere. The resulting mixture was stirred for 4 h at room temperature, then concentrated under vacuum to give a residue. The residue was purified by Prep-HPLC (Condition 2, Gradient 10) to afford 4-(5-{3-methoxy-4,6-dimethylpyrazolo[1,5-a]pyrazin-2-yl} thieno[2,3-c]pyrazol-2-yl) piperidine (17.0 mg, 42.51%) as a solid.

Compounds 208, 210, 211, 214, 216, 218, 219, 221-226, 228, and 248 were prepared according to the procedures herein and outlined in this Example 80. The table below provides intermediates used in these procedures and final compound characterization data.

| | | LCMS | |
|---|---|---|---|
| | | (ESI, m/z) | |
| Compound No. and Structure | Coupling Reagent | [M + H]⁺ | ¹H NMR δ |
| 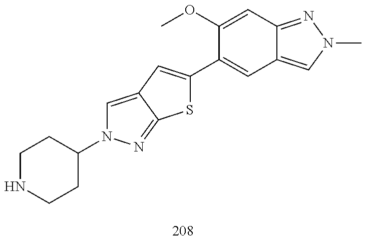 [SMILES: COc1cc2nn(C)cc2cc1-c1cc2cn(C3CCNCC3)nc2s1] | 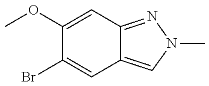 | 368 | (400 MHz, DMSO- d₆): δ 8.28 (s, 1H), 8.05 (s, 1H), 7.86 (s, 1H), 7.34 (s, 1H), 7.07 (s, 1H), 4.35 (tt, J = 11.5, 4.1 Hz, 1H), 4.12 (s, 3H), 3.90 (s, 3H), 3.06 (dt, J = 12.4, 3.3 Hz, 2H), 2.61 (td, J = 12.3, 2.5 Hz, 2H), 2.30 (s, 1H), 2.08-1.95 (m, 2H), 1.87 (qd, J = 12.0, 4.1 Hz, 2H) |
| 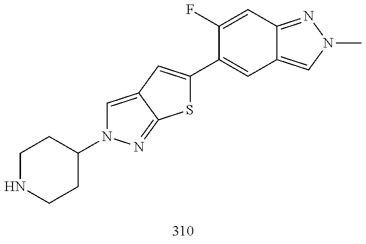 [SMILES: Cn1cc2cc(-c3cc4cn(C5CCNCC5)nc4s3)c(F)cc2n1] | 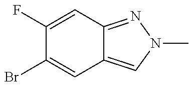 | 356 | (400 MHz, DMSO- d₆): δ 8.45 (s, 1H), 8.12 (s, 1H), 8.02 (d, J = 7.7 Hz, 1H), 7.49 (d, J = 12.7 Hz, 1H), 7.33 (d, J = 2.0 Hz, 1H), 4.37 (tt, J = 11.5, 4.1 Hz, 1H), 4.17 (s, 3H), 3.06 (dt, J = 12.5, 3.5 Hz, 2H), 2.61 (td, J = 12.3, 2.5 Hz, 2H), 2.28 (s, 1H), 2.01 (ddt, J = 11.4, 4.5, 2.2 Hz, |
| | | | 2H), 1.88 (qd, J = |
| | | | 12.0, 4.1 Hz, 2H) |
| 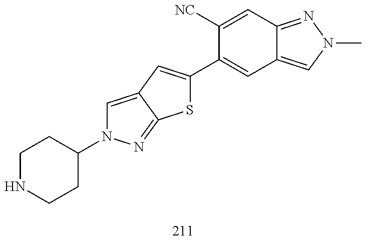 [SMILES: Cn1cc2cc(-c3cc4cn(C5CCNCC5)nc4s3)c(C#N)cc2n1] | 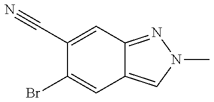 | 363 | (400 MHz, DMSO- d₆): δ 8.59 (s, 1H), 8.46 (s, 1H), 8.21 (s, 1H), 8.04 (s, 1H), 7.35 (s, 1H), 4.39 (ddt, J = 11.3, 8.1, 4.1 Hz, 1H), 4.28 (s, 3H), 3.07 (d, J = 12.7 Hz, 2H), 2.94 (s, 1H), 2.68-2.58 (m, 2H), 2.11-1.97 (m, 2H), 1.90 (qd, J = 11.9, 4.0 Hz, 2H) |
| 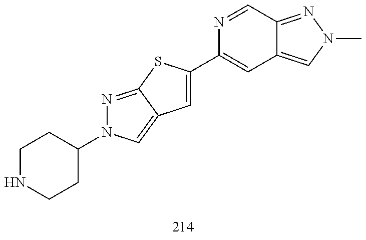 [SMILES: Cn1cc2cc(-c3cc4cn(C5CCNCC5)nc4s3)ncc2n1] | 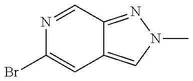 | 339 | (400 MHz, 353K, DMSO-d₆): δ 9.18 (s, 1H), 9.14 (t, J = 1.2 Hz, 1H), 8.97 (s, 1H), 8.47 (s, 1H), 8.12 (d, J = 1.4 Hz, 1H), 8.05 (s, 1H), 7.59 (s, 1H), 4.68 (p, J = 8.1, 7.5 Hz, 1H), 4.27 (s, 3H), 3.42 (d, J = 13.0 Hz, 2H), 3.11 (s, 2H), 2.31 (td, J = 8.8, 7.5, 4.2 Hz, 4H) |
| 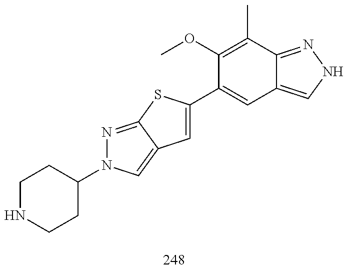 [SMILES: COc1c(-c2cc3cn(C4CCNCC4)nc3s2)cc2c[nH]nc2c1C] | 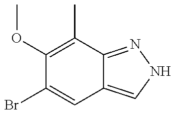 | 368 | (400 MHz, 353K, DMSO-d₆): δ 12.95 (s, 1H), 8.03 (s, 1H), 7.99 (s, 1H), 7.79 (s, 1H), 7.28 (s, 1H), 4.35 (tt, J = 11.5, 4.1 Hz, 1H), 3.65 (s, 3H), 3.13- 3.07 (m, 2H), 2.64 (td, J = 12.2, 2.6 Hz, 2H), 2.48 (s, 3H), 2.10-2.00 (m, 2H), 1.90 (qd, J = 11.9, 4.2 Hz, 3H) |
| 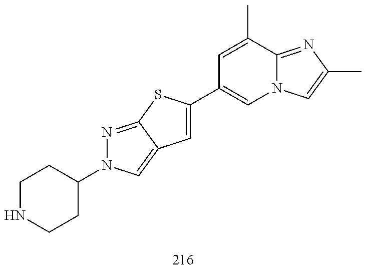 [SMILES: Cc1cn2cc(-c3cc4cn(C5CCNCC5)nc4s3)cc(C)c2n1] | 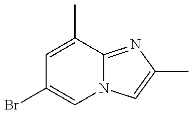 | 352 | (400 MHz, 353K, DMSO-d₆): δ 8.55 (d, J = 1.8 Hz, 1H), 8.03 (s, 1H), 7.67 (d, J = 1.0 Hz, 1H), 7.32 (d, J = 7.5 Hz, 2H), 4.35 (tt, J = 11.4, 4.1 Hz, 1H), 3.09 (dd, J = 7.8, 4.5 Hz, 2H), 2.64 (td, J = 12.2, 2.6 Hz, 2H), 2.51 (s, 3H), 2.35 (d, J = 0.8 Hz, 3H), 2.04 (ddt, J = 11.5, 4.4, 2.3 Hz, 2H), 1.89 (qd, J = 11.9, 4.2 |
| | | | Hz, 3H) |
| 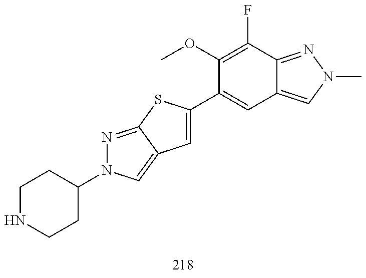 [SMILES: COc1c(-c2cc3cn(C4CCNCC4)nc3s2)cc2cn(C)nc2c1F] | 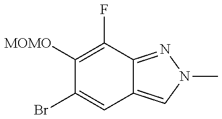 | 386 | (400 MHz, 353K, DMSO-d₆): δ 8.39 (d, J = 2.9 Hz, 1H), 8.02 (s, 1H), 7.73 (d, J = 1.0 Hz, 1H), 7.33 (s, 1H), 4.36 (tt, J = 11.4, 4.1 Hz, 1H), 4.18 (s, 3H), 3.89 (d, J = 1.3 Hz, 3H), 3.13- 3.06 (m, 2H), 2.64 (td, J = 12.3, 2.6 Hz, 2H), 2.09- 2.00 (m, 2H), 1.89 (qd, J = 11.9, 4.2 Hz, 3H) |
| 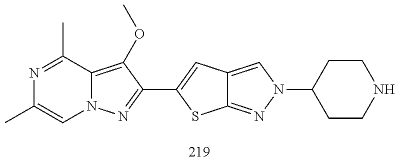 [SMILES: COc1c(-c2cc3cn(C4CCNCC4)nc3s2)nn2cc(C)nc(C)c12] | 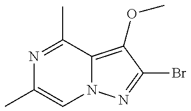 | 383 | (400 MHz, 353K, DMSO-d₆): δ 8.23 (s, 1H), 8.08 (s, 1H), 7.53 (s, 1H), 4.37 (tt, J = 11.4, 4.1 Hz, 1H), 3.92 (s, 3H), 3.14-3.07 (m, 2H), 2.76 (s, 3H), 2.64 (td, J = 12.3, 2.6 Hz, 2H), |
| | | | 2.38 (d, J = 1.1 Hz, |
| | | | 3H), 2.10-1.98 |
| | | | (m, 2H), 1.90 (qd, J = |
| | | | 11.8, 4.2 Hz, 3H) |
| 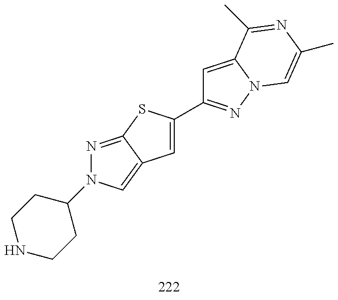 [SMILES: Cc1cn2nc(-c3cc4cn(C5CCNCC5)nc4s3)cc2c(C)n1] | 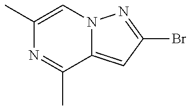 | 353 | (400 MHz, DMSO- d₆): δ 8.47 (s, 1H), 8.17 (s, 1H), 7.61 (s, 1H), 7.37 (s, 1H), 4.36 (t, J = 10.3 Hz, 1H), 3.07 (d, J = 12.2 Hz, 2H), 2.68 (s, 3H), 2.61 (t, J = 11.9 Hz, 2H), 2.41 (s, 3H), 2.01 (d, J = 11.9 Hz, 2H), 1.88 (qd, J = 11.9, 4.0 Hz, 2H) |
| 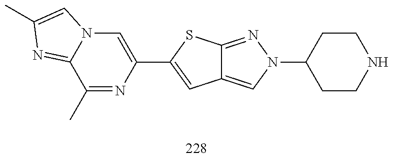 [SMILES: Cc1cn2cc(-c3cc4cn(C5CCNCC5)nc4s3)nc(C)c2n1] | 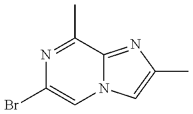 | 353 | (400 MHz, DMSO- d₆): δ 8.95 (s, 1H), 8.11 (s, 1H), 7.83 (s, 1H), 7.54 (s, 1H), 4.36 (tt, J = 11.7, 4.1 Hz, 1H), 3.15-2.99 (m, 2H), 2.73 (s, 3H), 2.62 (td, J = 12.4, 2.5 Hz, 2H), 2.41 |
| | | | (s, 3H), 2.12-1.96 |
| | | | (m, 2H), 1.89 (qd, J = |
| | | | 12.0, 4.1 Hz, 2H) |
| 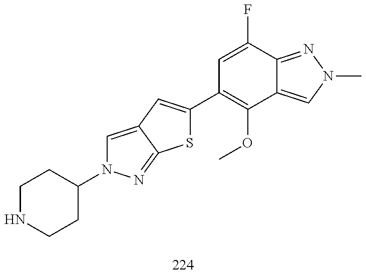 [SMILES: COc1c(-c2cc3cn(C4CCNCC4)nc3s2)cc(F)c2nn(C)cc12] | 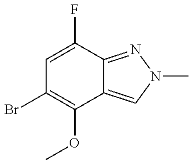 | 386 | (400 MHz, DMSO- d₆): δ 8.78 (d, J = 2.7 Hz, 1H), 8.06 (s, 1H), 7.48 (d, J = 12.8 Hz, 1H), 7.45 (s, 1H), 4.35 (tt, J = 11.5, 4.1 Hz, 1H), 4.19 (s, 3H), 4.03 (s, 3H), 3.12-3.00 (m, 2H), 2.61 (td, J = 12.4, 2.6 Hz, 2H), 2.07-1.95 (m, 2H), 1.88 (qd, J = 12.0, 4.1 Hz, 2H) |
| 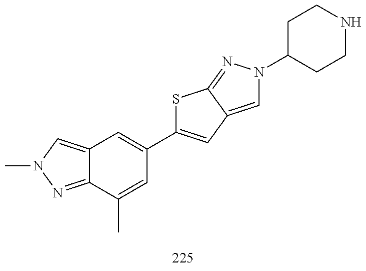 [SMILES: Cc1cc(-c2cc3cn(C4CCNCC4)nc3s2)cc2cn(C)nc12] | 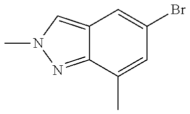 | 352 | (400 MHz, DMSO- d₆): δ 8.32 (s, 1H), 8.06 (s, 1H), 7.80- 7.65 (m, 1H), 7.39 (d, J = 21.2 Hz, 2H), 4.35 (tt, J = 11.4, 4.1 Hz, 1H), 4.17 (s, 3H), 3.15- 3.00 (m, 2H), 2.67- 2.58 (m, 2H), 2.54 (s, 3H), 2.09- 1.95 (m, 2H), 1.95- 1.78 (m, 2H) |
| 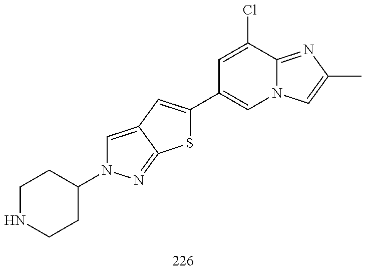 [SMILES: Cc1cn2cc(-c3cc4cn(C5CCNCC5)nc4s3)cc(Cl)c2n1] | 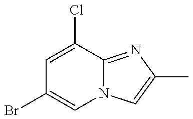 | 372 | (400 MHz, DMSO- d₆): δ 8.77 (d, J = 1.7 Hz, 1H), 8.15 (s, 1H), 7.84 (t, J = 1.7 Hz, 2H), 7.55 (s, 1H), 4.36 (tt, J = 11.5, 3.7 Hz, 1H), 3.06 (dt, J = 12.6, 3.4 Hz, 2H), 2.61 (td, J = 12.3, 2.5 Hz, 2H), 2.36 (s, 3H), 2.07-1.95 (m, 2H), 1.87 (qd, J = 12.0, 4.1 Hz, 2H) |
| 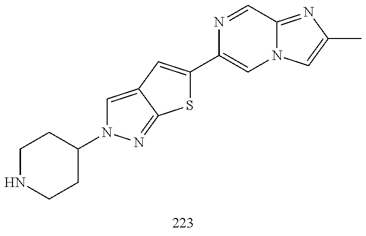 [SMILES: Cc1cn2cc(-c3cc4cn(C5CCNCC5)nc4s3)ncc2n1] | 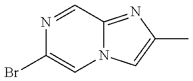 | 339 | (400 MHz, DMSO- d₆): δ 9.12 (s, 1H), 8.94 (s, 1H), 8.14 (s, 1H), 7.90 (s, 1H), 7.58 (d, J = 1.9 Hz, 1H), 4.37 (d, J = 12.1 Hz, 1H), 3.06 (d, J = 12.3 Hz, 2H), 2.61 (t, J = 12.3 Hz, 2H), 2.43 (s, 3H), 2.01 (d, J = 12.3 Hz, 2H), 1.87 (ddt, J = 15.8, 11.9, 6.5 Hz, 2H) |
| 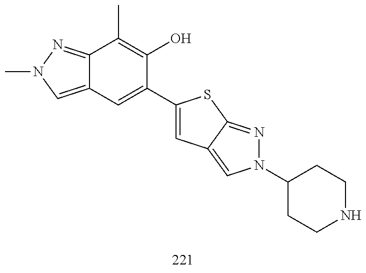 [SMILES: Cc1c(O)c(-c2cc3cn(C4CCNCC4)nc3s2)cc2cn(C)nc12] | 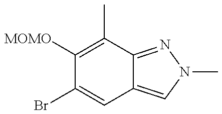 | 368 | (400 MHz, DMSO- d₆): δ 8.74 (s, 1H), 8.13 (d, J = 71.3 Hz, 2H), 7.67 (s, 1H), 7.36 (s, 1H), 4.34 (tt, J = 11.4, 4.1 Hz, 1H), 4.11 (s, 3H), 3.06 (dt, J = 12.5, 3.3 Hz, 2H), 2.63 (qd, J = 12.6, 2.2 Hz, 2H), 2.39 (s, 3H), 2.11-1.94 (m, 2H), 1.87 (qd, J = 12.0, 4.1 Hz, 2H) |

### Example 81: Synthesis of Compounds 207, 209, and 229

### Synthesis of Intermediate R16

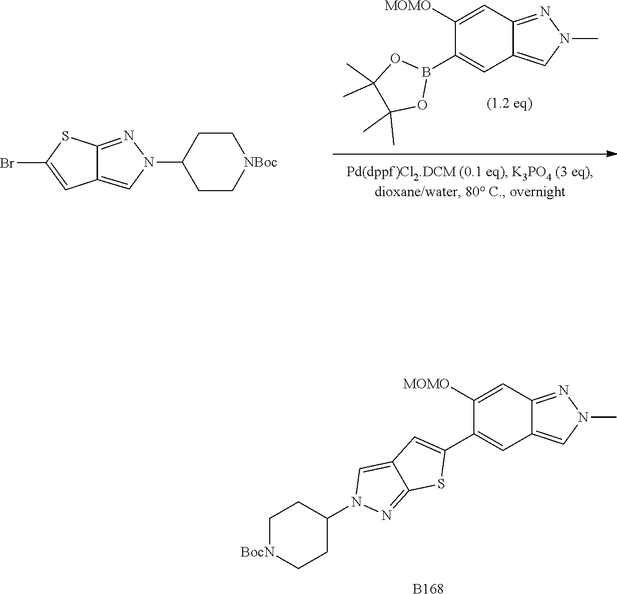

A mixture of tert-butyl 4-{5-bromothieno[2,3-c]pyrazol-2-yl}piperidine-1-carboxylate (100 mg, 0.259 mmol, 1.00 equiv), 6-(methoxymethoxy)-2-methyl-5-(4,4,5,5-tetramethyl-1,3,2-dioxaborolan-2-yl) indazole (98.84 mg, 0.311 mmol, 1.2 equiv), Pd(dppf)Cl₂·CH₂Cl₂ (21.09 mg, 0.026 mmol, 0.1 equiv), and K₃PO₄ (164.84 mg, 0.777 mmol, 3 equiv) in a mixture of 1,4-dioxane (2 mL) and water (0.4 mL, 22.203 mmol, 85.77 equiv) was stirred overnight at 80° C. under nitrogen atmosphere. The reaction mixture was cooled to room temperature, then diluted with water (10 mL) and extracted with ethyl acetate (3×15 mL). The organic layers were combined, washed with brine (1×10 mL), dried over anhydrous Na₂SO₄, and filtered. After filtration, the filtrate was concentrated under reduced pressure to give a residue. The residue was purified by silica gel column chromatography, eluted with DCM/EA (2:1) to afford tert-butyl 4-{5-[6-(methoxymethoxy)-2-methylindazol-5-yl]thieno[2,3-c]pyrazol-2-yl}piperidine-1-carboxylate (110 mg, 85.40%) as a solid.

### Synthesis of Compound 209

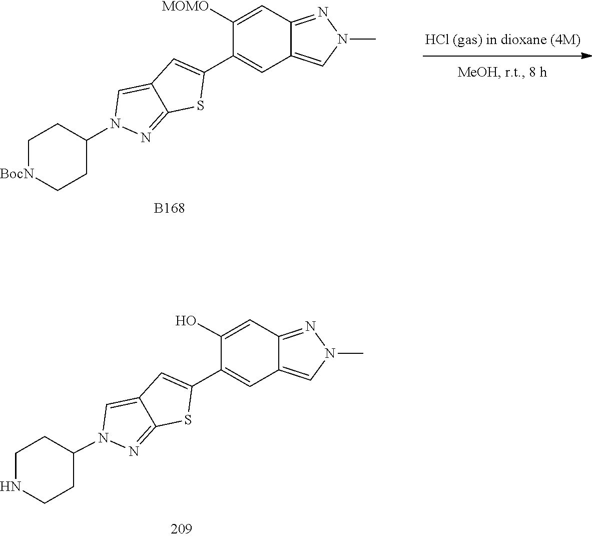

A mixture of tert-butyl 4-{5-[6-(methoxymethoxy)-2-methylindazol-5-yl]thieno[2,3-c]pyrazol-2-yl} piperidine-12-carboxylate (100 mg, 0.201 mmol, 1.00 equiv) and HCl (gas) in 1,4-dioxane (2.5 mL, 82.280 mmol, 409.43 equiv) in methanol (2.50 mL, 61.759 mmol, 307.26 equiv) was stirred for 8 h at room temperature under air atmosphere. The resulting mixture was concentrated under vacuum to give a residue. The residue was purified by Prep-TIPLC (Condition 2, Gradient 16) to afford 2-methyl-5-[2-(piperidin-4-yl) thieno[2,3-c]pyrazol-5-yl]indazol-6-ol (28.7 mg, 39.81H) as a solid.

Compounds 207, 209, and 229 were prepared according to the procedures described herein and outlined in this Example 80. The table below provides intermediates used in these procedures and final compound characterization data.

| | | LCMS | |
|---|---|---|---|
| | | (ESI, | |
| Compound No. and | Coupling | m/z) | |
| Structure | Reagent | [M + H]⁺ | ¹H NMR δ |
| 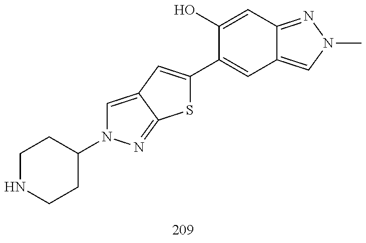 [SMILES: Cn1cc2cc(-c3cc4cn(C5CCNCC5)nc4s3)c(O)cc2n1] | 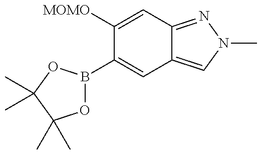 | 354 | (400 MHz, DMSO-d₆): δ 10.06 (s, 1H), 8.22 (s, 1H), 8.03 (s, 1H), 7.81 (s, 1H), 7.45 (s, 1H), 6.92 (s, 1H), 4.34 (dq, J = 11.3, 5.7, 4.2 Hz, 1H), 4.08 (s, 3H), 3.06 (d, J = 12.6 Hz, 2H), 2.64- 2.57 (m, 2H), 2.05-1.96 (m, 2H), 1.87 (qd, J = 12.0, 4.1 Hz, 2H) |
| 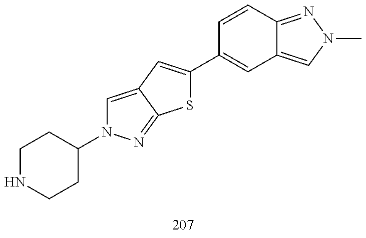 [SMILES: Cn1cc2cc(-c3cc4cn(C5CCNCC5)nc4s3)ccc2n1] | 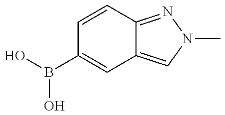 | 338 | (400 MHz, DMSO-d₆): δ 8.37 (s, 1H), 8.08 (s, 1H), 7.89 (d, J = 1.5 Hz, 1H), 7.64 (d, J = 1.4 Hz, 2H), 7.40 (s, 1H), 4.35 (tt, J = 11.5, 4.1 Hz, 1H), 4.18 (s, 3H), 3.06 (dt, J = 12.5, 3.4 Hz, 2H), 2.61 (td, J = 12.3, 2.5 Hz, 2H), 2.05-1.96 (m, 2H), 1.87 (qd, J = 12.0, 4.1 Hz, 2H) |
| 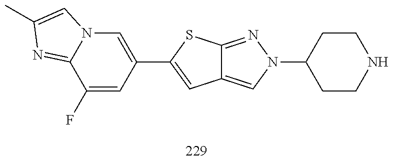 [SMILES: Cc1cn2cc(-c3cc4cn(C5CCNCC5)nc4s3)cc(F)c2n1] | 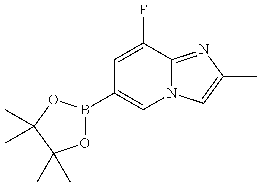 | 356 | (400 MHz, DMSO-d₆): δ 8.65 (d, J = 1.5 Hz, 1H), 8.15 (s, 1H), 7.87-7.81 (m, 1H), 7.60 (dd, J = 12.4, 1.6 Hz, 1H), 7.51 (s, 1H), 4.35 (ddt, J = 11.5, 8.1, 4.1 Hz, 1H), 3.06 (dt, J = 12.7, 3.3 Hz, 2H), 2.62 (qd, J = 13.2, 12.3, 2.2 Hz, 2H), 2.36 (s, 3H), 2.06-1.95 (m, 2H), 1.87 (qd, J = 12.0, 4.1 Hz, 2H) |

### Example 82: Synthesis of Compound 217

### Synthesis of Intermediate B169

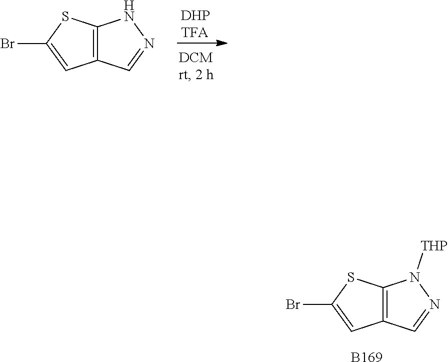

A solution of 5-bromo-1H-thieno[2,3-c] pyrazole (2.00 g, 9.36 mmol, 1.00 equiv) in DCM (20 mL) was treated with DHP (0.91 g, 10.29 mmol, 1.10 equiv) for 5 minutes at 25° C. under nitrogen atmosphere. To the reaction mixture was added TFA (0.06 g, 0.47 mmol, 0.05 equiv) dropwise at 25° C. The reaction mixture was stirred for 2 h, then extracted with ethyl acetate (2×50 mL). The organic layers were combined, washed with saturated salt solution (50 mL), dried over anhydrous Na₂SO₄, and filtered. After filtration, the filtrate was concentrated under reduced pressure to yield 5-bromo-1-(oxan-2-yl)thieno[2,3-c]pyrazole (2.80 g crude) as a solid. LCMS (ES, m/z): 287 [M+H]⁺.

### Synthesis of Intermediate B170

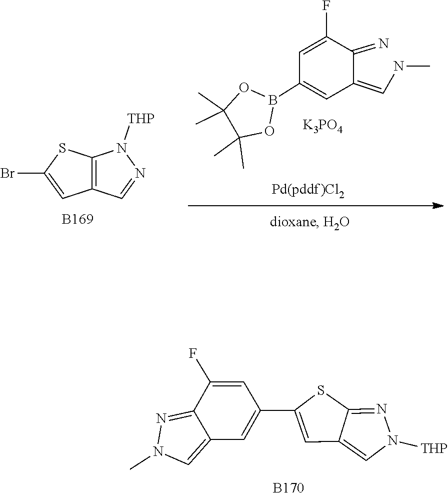

To a stirred mixture of 5-bromo-1-(oxan-2-yl) thieno[2,3-c] pyrazole (1.90 g, 6.62 mmol, 1.00 equiv) and 7-fluoro-2-methyl-5-(4,4,5,5-tetramethyl-1,3,2-dioxaborolan-2-yl) indazole (2.37 g, 8.60 mmol, 1.30 equiv) in 1,4-dioxane (19 mL) and water (3.80 mL) was added Pd(dtbpf)Cl₂ (0.43 g, 0.66 mmol, 0.10 equiv) and K₃PO₄ (4.21 g, 19.85 mmol, 3.00 equiv) at 100° C. under N₂ atmosphere. The reaction mixture was stirred overnight at 100° C., then extracted with ethyl acetate (2×50 mL). The organic layers were combined, washed with saturated salt solution (50 mL), dried over anhydrous Na₂SO₄, and filtered. After filtration, the filtrate was concentrated under reduced pressure to give a residue. The residue was purified by silica gel column chromatography, eluted with EA and PE (5:2) to afford 7-fluoro-2-methyl-5-[2-(oxan-2-yl) thieno[2,3-c] pyrazol-5-yl] indazole (1.8 g) as a solid. LCMS (ES, m/z): 357 [M+H]⁺.

### Synthesis of Intermediate B171

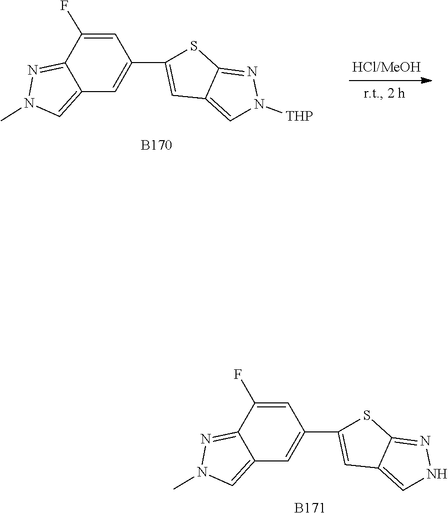

A solution of 7-fluoro-2-methyl-5-[2-(oxan-2-yl) thieno[2,3-c] pyrazol-5-yl] indazole (1.80 g) in HCl (gas) in 1,4-dioxane (18 mL) and methanol (18 mL) was stirred for 2 h at 25° C. under N₂ atmosphere. The reaction mixture was concentrated under reduced pressure to afford 7-fluoro-2-methyl-5-{2H-thieno[2,3-c] pyrazol-5-yl} indazole (2 g) as a solid. LCMS (ES, m/z): 273 [M+H]⁺.

### Synthesis of Intermediate B172 and B173

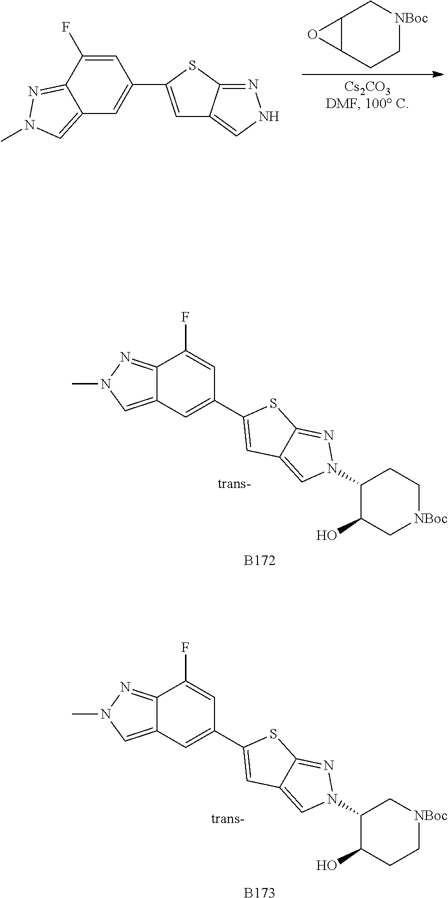 [SMILES: Cn1cc2cc(-c3cc4c[nH]nc4s3)cc(F)c2n1 ; CC(C)(C)OC(=O)N1CCC2OC2C1 ; "B172" ; "B173" ; Cn1cc2cc(-c3cc4cn([C@@H]5CCN(C(=O)OC(C)(C)C)C[C@H]5O)nc4s3)cc(F)c2n1 ; Cn1cc2cc(-c3cc4cn([C@@H]5CN(C(=O)OC(C)(C)C)CC[C@H]5O)nc4s3)cc(F)c2n1]

To a stirred solution of 7-fluoro-2-methyl-5-{2H-thieno[2,3-c] pyrazol-5-yl}indazole (200 mg, 0.734 mmol, 1.00 equiv) and tert-butyl 7-oxa-3-azabicyclo[4.1.0]heptane-3-carboxylate (175.62 mg, 0.881 mmol, 1.2 equiv) in DMF (4 mL) was added Cs₂CO₃ (717.93 mg, 2.202 mmol, 3 equiv) in portions at room temperature under nitrogen atmosphere. The resulting mixture was stirred overnight at 100° C. under nitrogen atmosphere. The resulting mixture was concentrated under vacuum to give a residue. The residue was purified by silica gel column chromatography, eluted with PE/EA (1:1), followed by chiral-Prep-HPLC (Condition 5, Gradient 1) to afford tert-butyl (3R,4R)-4-[5-(7-fluoro-2-methylindazol-5-yl)thieno[2,3-c]pyrazol-2-yl]-3-hydroxypiperidine-1-carboxylate (20 mg, 5.77%) and tert-butyl (3R,4R)-3-[5-(7-fluoro-2-methylindazol-5-yl)thieno [2,3-c]pyrazol-2-yl]-4-hydroxypiperidine-1-carboxylate (15 mg, 4.33%) as solids.

### Synthesis of Compound 249

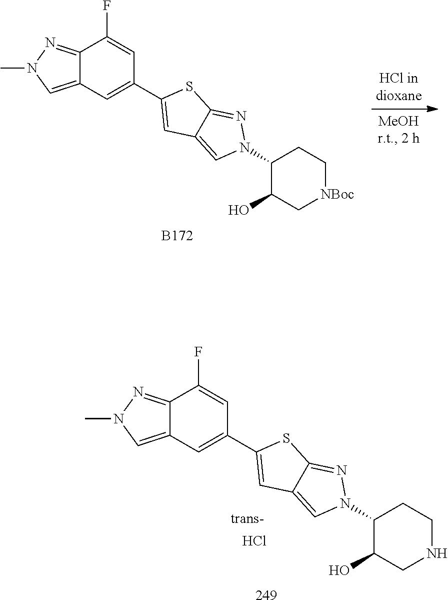 [SMILES: "B172" ; Cn1cc2cc(-c3cc4cn([C@@H]5CCN(C(=O)OC(C)(C)C)C[C@H]5O)nc4s3)cc(F)c2n1 ; Cn1cc2cc(-c3cc4cn([C@@H]5CCNC[C@H]5O)nc4s3)cc(F)c2n1 ; Cl ; "249"]

To a stirred solution of tert-butyl (3R,4R)-4-[5-(7-fluoro-2-methylindazol-5-yl)thieno[2,3-c]pyrazol-2-yl]-3-hydroxypiperidine-1-carboxylate (20 mg, 0.042 mmol, 1.00 equiv) in methanol (1 mL) was added HCl (gas) in 1,4-dioxane (0.5 mL) dropwise at room temperature under nitrogen atmosphere. The resulting mixture was stirred for 2 h at room temperature under nitrogen atmosphere, then concentrated under vacuum to afford (3R,4R)-4-[5-(7-fluoro-2-methylindazol-5-yl)thieno[2,3-c]pyrazol-2-yl] piperidin-3-ol (9.6 mg, 60.94%) as a solid. LCMS (ES, m/z): 372 [M+H]⁺. ¹H NMR (400 MHz, DMSO-d₆) δ 8.95 (s, 2H), 8.50 (d, J=2.8 Hz, 1H), 8.06 (s, 1H), 7.73 (d, J=1.4 Hz, 1H), 7.53 (dd, J=13.0, 1.5 Hz, 1H), 7.49 (s, 1H), 4.37 (td, J 11.6, 10.6, 4.2 Hz, 1H), 4.21 (s, 3H), 4.13 (tt, J=10.3, 5.2 Hz, 1H), 3.48-3.38 (m, 1H), 3.33 (s, 2H), 3.07 (t, J=12.5 Hz, 1H), 2.84 (t, J=11.5 Hz, 1H), 2.41-2.32 (m, 1H), 2.20 (d, J=13.7 Hz, 1H).

### Synthesis of Compound 217

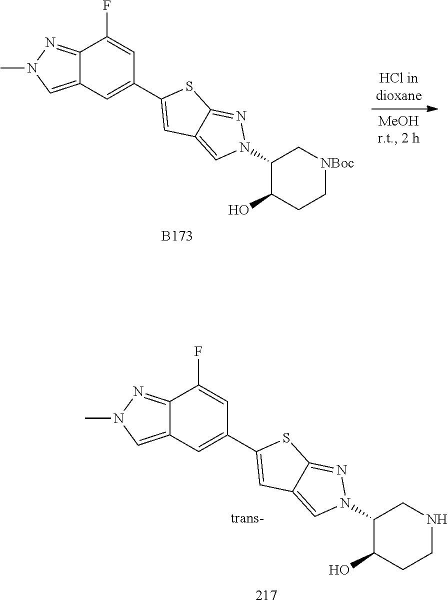 [SMILES: "B173" ; Cn1cc2cc(-c3cc4cn([C@@H]5CN(C(=O)OC(C)(C)C)CC[C@H]5O)nc4s3)cc(F)c2n1 ; Cn1cc2cc(-c3cc4cn([C@@H]5CNCC[C@H]5O)nc4s3)cc(F)c2n1 ; "217"]

To a stirred solution of tert-butyl (3R,4R)-3-[5-(7-fluoro-2-methylindazol-5-yl)thieno[2,3-c]pyrazol-1-yl]-4-hydroxypiperidine-1-carboxylate (15 mg, 0.032 mmol, 1.00 equiv) in methanol (0.5 mL) was added HCl (gas) in 1,4-dioxane (0.5 mL) dropwise at room temperature under nitrogen atmosphere. The resulting mixture was stirred for 2 h at room temperature under nitrogen atmosphere, then concentrated under vacuum to give a residue. The residue was purified by Prep-HPLC (Condition 2, Gradient 7) to afford (3R,4R)-3-[5-(7-fluoro-2-methylindazol-5-yl)thieno[2,3-c]pyrazol-1-yl]piperidin-4-ol (1.9 mg, 16.08%) as a solid. LCMS (ES, m/z): 372 [M+H]⁺. 1H NMR (400 MHz, DMSO-d₆) δ 8.48 (d, J=2.8 Hz, 1H), 8.07 (s, 1H), 7.71 (d, J=1.4 Hz, 1H), 7.52 (dd, J=13.1, 1.5 Hz, 1H), 7.48 (s, 1H), 4.95 (d, J=5.7 Hz, 1H), 4.21 (s, 3H), 4.00 (td, J=10.0, 4.3 Hz, 1H), 3.90 (dq, J=9.9, 5.1 Hz, 1H), 3.13 (dd, J=12.2, 4.4 Hz, 1H), 2.94 (d, J=12.7 Hz, 1H), 2.86 (t, J=11.5 Hz, 1H), 2.68 (p, J=1.8 Hz, 1H), 2.55 (s, 1H), 1.93 (d, J=8.5 Hz, 1H), 1.42 (qd, J=12.3, 4.3 Hz, 1H).

### Example 83: Synthesis of Compound 246

### Synthesis of Intermediate B174

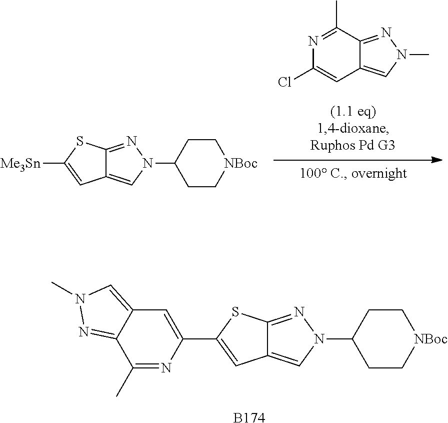

A mixture of tert-butyl 4-[5-(trimethylstannyl)thieno[2,3-c]pyrazol-2-yl]piperidine-1-carboxylate (160 mg, 0.340 mmol, 1.00 equiv), 5-chloro-2,7-dimethylpyrazolo[3,4-c]pyridine (67.98 mg, 0.374 mmol, 1.1 equiv), and RuPhos Palladacycle Gen.3 (28.46 mg, 0.034 mmol, 0.1 equiv) in 1,4-dioxane (3 mL) was stirred overnight at 100° C. under nitrogen atmosphere. The resulting mixture was concentrated under reduced pressure to give a residue. The residue was purified by reverse flash chromatography (Condition 1, Gradient 2) to afford tert-butyl 4-(5-{2,7-dimethylpyrazolo[3,4-c]pyridin-5-yl} thieno[2,3-c] pyrazol-2-yl) piperidine-1-carboxylate (65 mg, 29.55%) as a solid. LCMS (ESI, m z): 453 [M+H]⁺.

### Synthesis of Compound 246

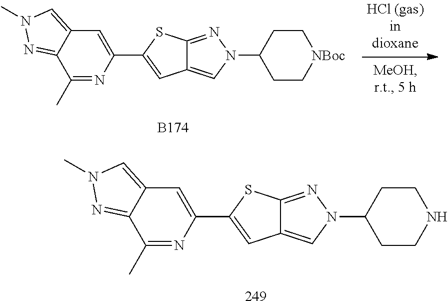 [SMILES: "B174" ; Cc1nc(-c2cc3cn(C4CCN(C(=O)OC(C)(C)C)CC4)nc3s2)cc2cn(C)nc12 ; Cc1nc(-c2cc3cn(C4CCNCC4)nc3s2)cc2cn(C)nc12 ; "249"]

To a stirred solution of tert-butyl 4-(5-{2,7-dimethylpyrazolo[3,4-c]pyridin-5-yl}thieno[2,3-c]pyrazol-2-yl)piperidine-1-carboxylate (65 mg, 0.144 mmol, 1 equiv) in methanol (3.25 mL) was added HCl (gas) in 1,4-dioxane (3.25 mL) dropwise at room temperature under air atmosphere. The resulting mixture was stirred for 5 h at room temperature under air atmosphere. The resulting mixture was concentrated under vacuum to give a residue. The residue was purified by Prep-HPLC (Condition 1, Gradient 4) to afford 4-(5-{2,7-dimethylpyrazolo[3,4-c]pyridin-5-yl}thieno[2,3-c]pyrazol-2-yl)piperidine hydrochloride (12.1 mg, 21.36%) as a solid. LCMS (ESI, m z): 353[M+H]. ¹H NMR (400 MHz, DMSO-d₆): δ 9.28 (s, 1H), 9.08 (s, 1H), 8.45 (s, 1H), 8.07 (s, 1H), 7.96 (s, 1H), 7.64 (s, 1H), 4.69 (td, J=9.3, 4.7 Hz, 1H), 4.28 (s, 3H), 3.43 (d, J=12.7 Hz, 2H), 3.12 (q, J=12.5, 12.0 Hz, 2H), 2.84 (s, 3H), 2.34 (td, J=9.8, 4.2 Hz, 4H).

### Example 84: Synthesis of Compound 243

### Synthesis of Intermediate B175

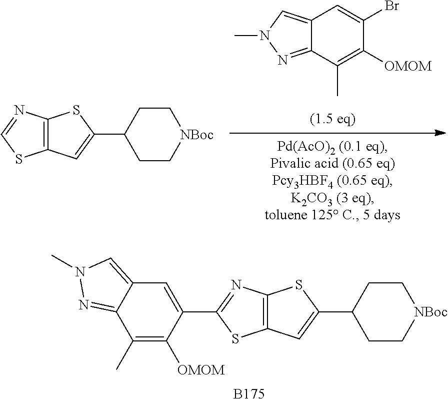

To a stirred mixture of tert-butyl 4-{thieno[2,3-d][1,3]thiazol-5-yl}piperidine-1-carboxylate (100 mg, 0.31 mmol, 1.00 equiv), tert-butyl 4-{thieno[2,3-d][1,3]thiazol-5-yl}piperidine-1-carboxylate (100 mg, 0.31 mmol, 1.00 equiv), 5-bromo-6-(methoxymethoxy)-2,7-dimethylindazole (131.82 mg, 0.46 mmol, 1.50 equiv), Pd(OAc)₂ (6.92 mg, 0.03 mmol, 0.10 equiv) and PCy₃HBF₄ (73.77 mg, 0.20 mmol, 0.65 equiv) in toluene (3 mL) was added pivalic acid (20.46 mg, 0.20 mmol, 0.65 equiv) and K₂CO₃ (127.79 mg, 0.92 mmol, 3.00 equiv). The reaction mixture was stirred for 5 days at 125° C. under nitrogen atmosphere, then extracted with ethyl acetate (3×10 mL). The organic layers were combined, washed with saturated NaCl (1×10 mL), dried over anhydrous Na₂SO₄, and filtered. After filtration, the filtrate was concentrated under reduced pressure to give a residue. The residue was purified by silica gel column chromatography, eluted with PE/EA (1:1) to afford tert-butyl 4-{2-[6-(methoxymethoxy)-2,7-dimethylindazol-5-yl]thieno[2,3-d][1,3]thiazol-5-yl}piperidine-1-carboxylate (100 mg, 61.37%) as a solid. LCMS (ES, m/z):529 [M+H]⁺.

### Synthesis of Compound 243

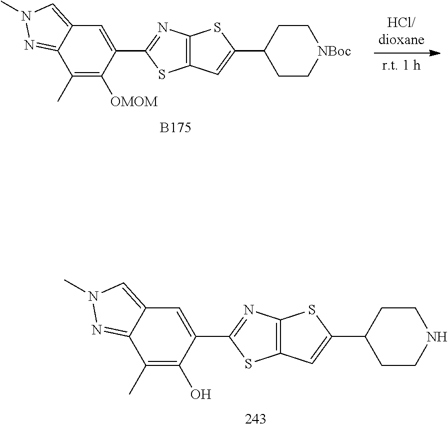

A solution oftert-butyl 4-{2-[6-(methoxymethoxy)-2,7-dimethylindazol-5-yl]thieno[2,3-d][1,3]thiazo 1-5-yl}piperidine-1-carboxylate (100 mg, 0.12 mmol, 1.00 equiv) and HCl (gas) in 1,4-dioxane (5 mL) was stirred for 1 h at room temperature, then concentrated under reduced pressure to give a residue. The residue was purified by reverse flash chromatography (Condition 2, Gradient 17) to afford 2,7-dimethyl-5-[5-(piperidin-4-yl)thieno[2,3-d][1,3]thiazol-2-yl]indazol-6-ol (41.90 mg, 57.61%) as a solid. LCMS (ES, m/z):385 [M+H]⁺. ¹H NMR (400 MHz, DMSO-d₆) δ 8.38 (d, J=17.1 Hz, 1H), 8.29 (s, 1H), 7.28 (d, J=1.0 Hz, 1H), 4.14 (s, 3H), 3.15 (m, 2H) 2.99 (m, 1H), 2.69 (td, J=12.1, 2.3 Hz, 2H), 2.40 (s, 3H), 2.03-1.94 (m, 2H), 1.62 (qd, J=12.4, 3.8 Hz, 2H).

### Example 85: Synthesis of Compound 101

### Synthesis of Intermediate B176

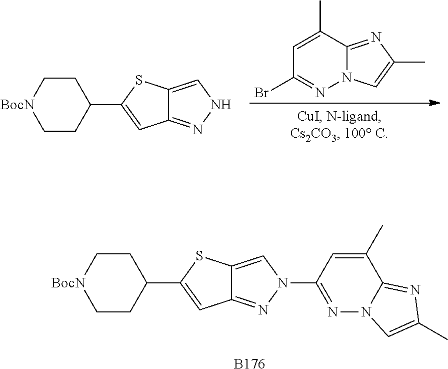 [SMILES: "B176" ; CC(C)(C)OC(=O)N1CCC(c2cc3n[nH]cc3s2)CC1 ; Cc1cn2nc(-n3cc4sc(C5CCN(C(=O)OC(C)(C)C)CC5)cc4n3)cc(C)c2n1 ; Cc1cn2nc(Br)cc(C)c2n1]

To a stirred mixture of tert-butyl 4-[2H-thieno[3,2-c]pyrazol-5-yl]piperidine-1-carboxylate (200.00 mg, 0.651 mmol, 1.00 equiv) and 6-bromo-2,8-dimethylimidazo[1,2-b]pyridazine (147.09 mg, 0.651 mmol, 1.00 equiv) in dioxane (5.00 mL) was added CuI (61.95 mg, 0.325 mmol, 0.50 equiv), (1S,2S)—N1,N2-dimethylcyclohexane-1,2-diamine (46.27 mg, 0.325 mmol, 0.50 equiv) and Cs₂CO₃ (635.93 mg, 1.952 mmol, 3.00 equiv) at 100° C. under N₂ atmosphere. The reaction mixture was stirred for 15 h at 100° C., then concentrated under reduced pressure to give a residue. The residue was purified by silica gel column chromatography, eluted with PE:EA (1:1), followed by Prep-HPLC (Condition 1, Gradient 3) to afford tert-butyl 4-(2-[2,8-dimethylimidazo[1,2-b]pyridazin-6-yl]thieno[3,2-c]pyrazol-5-yl)piperidine-1-carboxylate (80 mg) as a solid. LCMS (ES, m/z): 453 [M+H]⁺.

### Synthesis of Compound 101

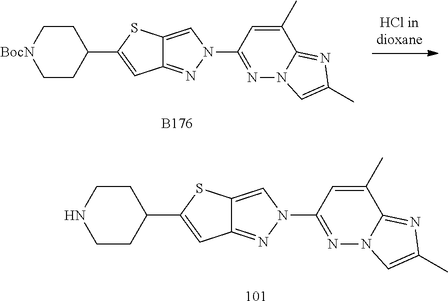 [SMILES: "B176" ; Cc1cn2nc(-n3cc4sc(C5CCN(C(=O)OC(C)(C)C)CC5)cc4n3)cc(C)c2n1 ; Cc1cn2nc(-n3cc4sc(C5CCNCC5)cc4n3)cc(C)c2n1 ; "101"]

To a stirred solution of tert-butyl 4-(2-[2,8-dimethylimidazo[1,2-b]pyridazin-6-yl]thieno[3,2-c]pyrazol-5-yl)piperidine-1-carboxylate (80.00 mg, 1 equiv) in methanol (5.00 mL) was added HCl (gas) in 1,4-dioxane (2.00 mL) at room temperature. The reaction mixture was stirred for 2 h at room temperature, then concentrated under reduced pressure to give a residue. The residue was purified by Prep-HPLC (Condition 1, Gradient 3) to afford 4-(2-[2,8-dimethylimidazo[1,2-b]pyridazin-6-yl]thieno[3,2-c]pyrazol-5-yl)piperidine (36.4 mg, 58.42%) as a solid. LCMS (ES, m z): 353 [M+H]⁺. 1H NMR (400 MHz, DMSO-d6) δ 8.13 (s, 1H), 8.08 (s, 1H), 7.69 (s, 1H), 7.56 (s, 1H), 3.02 (q, J=11.8 Hz, 3H), 2.61 (s, 4H), 2.59 (s, 1H), 2.39 (s, 3H), 1.96 (d, J=12.5 Hz, 2H), 1.62 (dd, J=12.3, 3.9 Hz, 1H), 1.56 (dd, J=12.1, 4.0 Hz, 1H).

### Example 86: Synthesis of Compound 102

### Synthesis of Intermediate B177

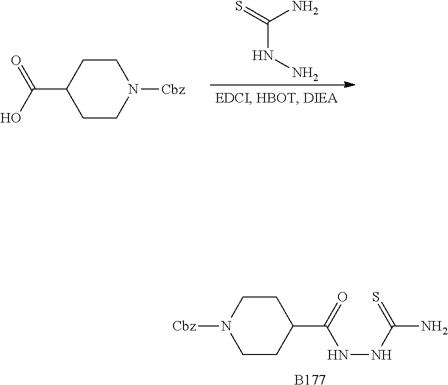

To a stirred solution of 1-[(benzyloxy)carbonyl]piperidine-4-carboxylic acid (10.00 g, 37.981 mmol, 1.00 equiv) and thiosemicarbazide (3.46 g, 37.9 mmol, 1.0 equiv) in DMF (100 mL) was added EDC.HCl (7.28 g, 37.98 mmol, 1.0 equiv), HOBT (5.13 g, 37.98 mmol, 1.0 equiv), and DIEA (14.73 g, 113.94 mmol, 3.0 equiv) at room temperature. The reaction mixture was stirred 3 h at room temperature, then diluted with water, and acidified to pH 5-6 with 1M HCl to form a precipitate. The precipitated solid was collected by filtration and washed with water to afford benzyl 4-(carbamothioylaminocarbamoyl)piperidine-1-carboxylate (12 g, 93.92%). LCMS (ES, m/z): 337 [M+H]⁺.

### Synthesis of Intermediate B178

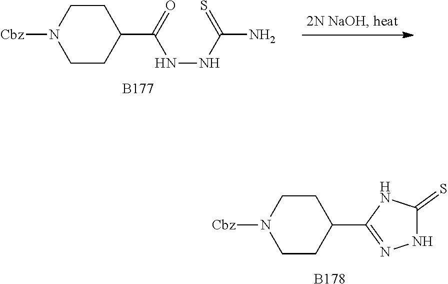 [SMILES: "B177" ; "B178" ; NC(=S)NNC(=O)C1CCN(C(=O)OCc2ccccc2)CC1 ; O=C(OCc1ccccc1)N1CCC(c2n[nH]c(=S)[nH]2)CC1]

Benzyl 4-(carbamothioylaminocarbamoyl)piperidine-1-carboxylate (12.0 g, 35.67 mmol, 1.0 equiv) and NaOH (1M) (100 mL) were combined at room temperature. The resulting mixture was stirred for 1 h at 50° C., then acidified to pH 5 with HCl (1M). The resulting mixture was extracted with CH₂Cl₂ (3×300 mL). The organic layers were combined, washed with brine (100 mL), dried over anhydrous Na₂SO₄, and filtered. After filtration, the filtrate was concentrated under reduced pressure to give a residue. The residue was purified by silica gel column chromatography, eluted with CH₂Cl₂/MeOH (50:1) to afford benzyl 4-(5-sulfanylidene-1,4-dihydro-1,2,4-triazol-3-yl)piperidine-1-carboxylate (7 g, 61.6%) as a solid. LCMS (ES, m/z): 319 [M+H]⁺.

### Synthesis of Intermediate B179

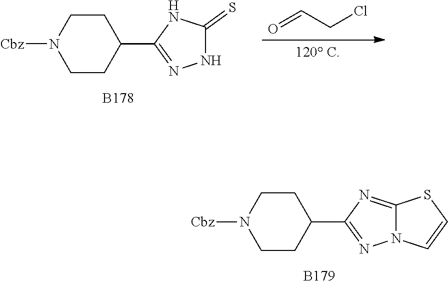

Benzyl 4-(5-sulfanylidene-1,4-dihydro-1,2,4-triazol-3-yl)piperidine-1-carboxylate (7.0 g, 21.98 mmol, 1.0 equiv) and 1,4-dioxane (60 mL) were combined in a pressure tank vessel at room temperature. To the reaction mixture was added chloroacetaldehyde (3.45 g, 43.97 mmol, 2.0 equiv) dropwise. The resulting mixture was stirred for 4 h at 125° C., then concentrated under reduced pressure to give a residue. The residue was purified by silica gel column chromatography, eluted with CH₂Cl₂/MeOH (40:1) to afford benzyl 4-[[1,2,4]triazolo[3,2-b][1,3]thiazol-2-yl]piperidine-1-carboxylate(1.3 g,17.2%) as a solid. LCMS (ES, m/z): 343 [M+H]⁺.

### Synthesis of Intermediate B180

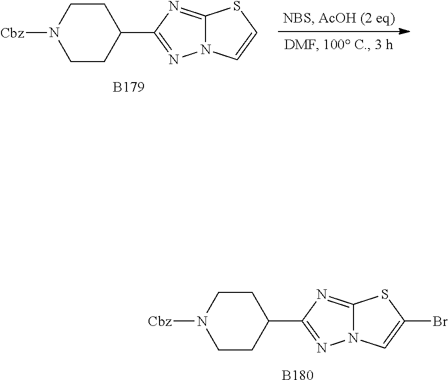 [SMILES: "B179" ; "B180" ; O=C(OCc1ccccc1)N1CCC(c2nc3sccn3n2)CC1 ; O=C(OCc1ccccc1)N1CCC(c2nc3sc(Br)cn3n2)CC1]

To a stirred solution of benzyl 4-[[1,2,4]triazolo[3,2-b][1,3]thiazol-2-yl]piperidine-1-carboxylate (800 mg, 2.3 mmol, 1.0 equiv) in DMF (6 mL) was added NBS (623 mg, 3.5 mmol, 1.5 equiv) and AcOH (28 mg, 0.4 mmol, 0.2 equiv) dropwise at room temperature. The resulting mixture was stirred for 16 h at 100° C. under nitrogen atmosphere, then quenched with water at room temperature. The resulting mixture was extracted with ethyl actate (100 mL). The organic layers were washed with brine (4×20 mL), dried over anhydrous Na₂SO₄, and filtered. After filtration, the filtrate was concentrated under reduced pressure to give a residue. The residue was purified by silica gel column chromatography, eluted with CH₂Cl₂/EtOAc (1:1) to afford benzyl 4-[5-bromo-[1,2,4]triazolo[3,2-b][1,3]thiazol-2-yl]piperidine-1-carboxylate (370 mg, 37.6%) as a solid. LCMS (ES, m/z): 422[M+2]⁺.

### Synthesis of Intermediate B181

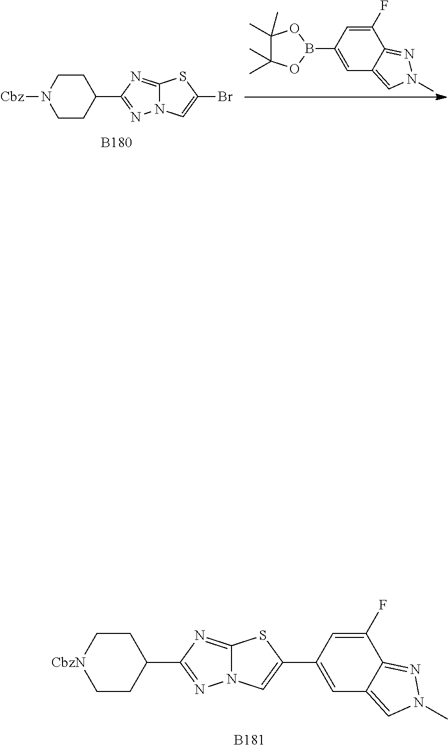

To a mixture of benzyl 4-[5-bromo-[1,2,4]triazolo[3,2-b][1,3]thiazol-2-yl]piperidine-1-carboxylate (50 mg, 0.12 mmol, 1.0 equiv) and 7-fluoro-2-methyl-5-(4,4,5,5-tetramethyl-1,3,2-dioxaborolan-2-yl)indazole (39 mg, 0.14 mmol, 1.2 equiv) in DMF (4 mL) and water (1 mL) was added K₃PO₄ (75 mg, 0.3 mmol, 3.0 equiv) and Pd(dppf)Cl₂ (17 mg, 0.02 mmol, 0.2 equiv). The reaction mixture was stirred for 3 h at 80° C. under a nitrogen atmosphere, then concentrated under reduced pressure and extracted with ethyl acetate (2×30 mL). The organic layers were combined, washed with brine (10 mL), dried over anhydrous Na₂SO₄, and filtered. After filtration, the filtrate was concentrated under reduced pressure to give a residue. The residue was purified by silica gel column chromatography, eluted with CH₂Cl₂/MeOH (30:1) to afford tert-butyl 4-[5-(7-fluoro-2-methylindazol-5-yl)-[1,2,4]triazolo[3,2-b][1,3]thiazol-2-yl]piperidine-1-carboxylate(35 mg,21.5%) as a solid. LCMS (ES, m/z): 491 [M+H]⁺.

### Synthesis of Compound 102

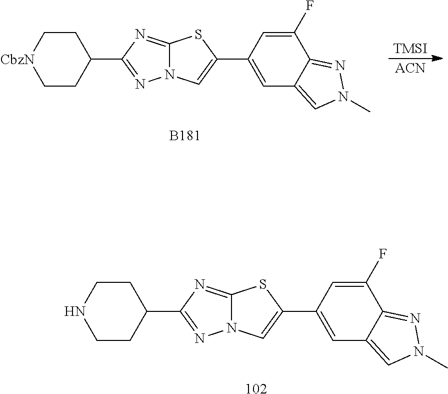

To a stirred solution of benzyl 4-[5-(7-fluoro-2-methylindazol-5-yl)-[1,2,4]triazolo[3,2-b][1,3]thiazol-2-yl]piperidine-1-carboxylate (35 mg, 0.07 mmol, 1.0 equiv) in acetonitrile (2.00 mL), was added TMSI (21 mg, 0.107 mmol, 1.5 equiv) dropwise at room temperature. The resulting mixture was stirred for 15 min at 70° C., then quenched with methanol at room temperature and concentrated under vacuum to give a residue. The residue was purified by silica gel column chromatography, eluted with CH₂Cl₂/MeOH (30:1) to afford 7-fluoro-2-methyl-5-[2-(piperidin-4-yl)-[1,2,4]triazolo[3,2-b][1,3]thiazol-5-yl]indazole (10.1 mg, 39.6%) as a solid. LCMS (ES, m/z): 357 [M+H]⁺. ¹H-NMR (400 MHz, DMSO-d6) δ 8.86 (s, 1H), 8.56 (d, J=2.8 Hz, 1H), 7.83 (d, J=1.4 Hz, 1H), 7.57 (dd, J=12.7, 1.5 Hz, 1H), 4.23 (s, 3H), 3.02 (dt, J=12.3, 3.5 Hz, 2H), 2.88 (tt, J=11.4, 3.8 Hz, 1H), 2.62 (td, J=12.0, 2.6 Hz, 2H), 1.92 (dd, J=13.5, 3.4 Hz, 2H), 1.65 (qd, J=11.8, 3.8 Hz, 2H).

### Example 87: Synthesis of Compound 114

### Synthesis of Intermediate B182

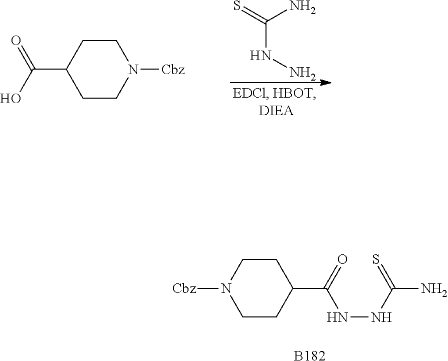 [SMILES: "B182" ; O=C(O)C1CCN(C(=O)OCc2ccccc2)CC1 ; NC(=S)NNC(=O)C1CCN(C(=O)OCc2ccccc2)CC1 ; NNC(N)=S]

To a stirred solution of 1-[(benzyloxy)carbonyl]piperidine-4-carboxylic acid (10.00 g, 37.981 mmol, 1.00 equiv.) and thiosemicarbazide (3.46 g, 37.9 mmol, 1.0 equiv.) in DMF (100 mL) was added EDC.HCl (7.28 g, 37.98 mmol, 1.0 equiv.), HOBT (5.13 g, 37.98 mmol, 1.0 equiv.), and DIEA (14.73 g, 113.94 mmol, 3.0 equiv.) at room temperature. The reaction mixture was stirred 3 h at room temperature, then diluted with water, and acidified to pH 5-6 with 1M HCl. A precipitate formed that was collected by filtration and washed with water to afford benzyl 4-(carbamothioylaminocarbamoyl)piperidine-1-carboxylate (12 g, 93.92%) as a solid. LCMS (ES, m/z): 337 [M+H]⁺.

### Synthesis of Intermediate B183

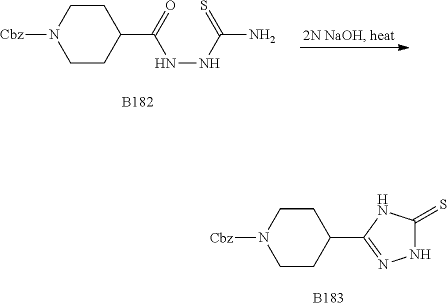 [SMILES: "B182" ; "B183" ; NC(=S)NNC(=O)C1CCN(C(=O)OCc2ccccc2)CC1 ; O=C(OCc1ccccc1)N1CCC(c2n[nH]c(=S)[nH]2)CC1]

A mixture of benzyl 4-(carbamothioylaminocarbamoyl)piperidine-1-carboxylate (12.0 g, 35.67 mmol, 1.0 equiv.) and NaOH (1M) (100 mL) was stirred for 1 h at 50° C., then acidified to pH 5 with HCl (1M). The resulting mixture was extracted with CH₂Cl₂ (3×300 mL). The organic layers were combined washed with brine (100 mL), dried over anhydrous Na₂SO₄, and filtered. After filtration, the filtrate was concentrated under reduced pressure to give a residue. The residue was purified by silica gel column chromatography, eluted with CH₂Cl₂/MeOH (50:1) to afford benzyl 4-(5-sulfanylidene-1,4-dihydro-1,2,4-triazol-3-yl)piperidine-1-carboxylate (7 g, 61.6%) as a solid. LCMS (ES, m/z): 319 [M+H]⁺

### Synthesis of Intermediate B184

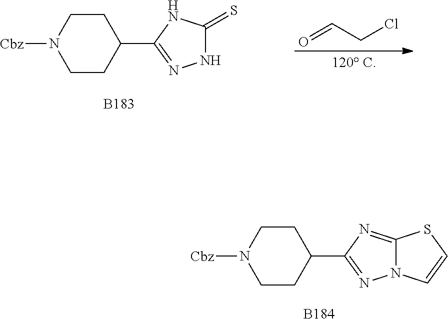

Benzyl 4-(5-sulfanylidene-1,4-dihydro-1,2,4-triazol-3-yl)piperidine-1-carboxylate (7.0 g, 21.98 mmol, 1.0 equiv.) and 1,4-dioxane (60 mL) were combined in a pressure tank vessel at room temperature. To the reaction mixture was added chloroacetaldehyde (3.45 g, 43.97 mmol, 2.0 equiv.) dropwise. The resulting mixture was stirred for 4 h at 125° C., then concentrated under reduced pressure to give a residue. The residue was purified by silica gel column chromatography, eluted with CH₂Cl₂/MeOH (40:1) to afford benzyl 4-[[1,2,4]triazolo[3,2-b][1,3]thiazol-2-yl]piperidine-1-carboxylate(1.3 g,17.2%) as a solid. LCMS (ES, m/z): 343 [M+H]⁺.

### Synthesis of Intermediate B185

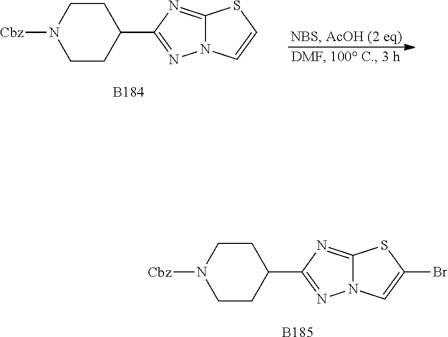 [SMILES: "B184" ; "B185" ; O=C(OCc1ccccc1)N1CCC(c2nc3sccn3n2)CC1 ; O=C(OCc1ccccc1)N1CCC(c2nc3sc(Br)cn3n2)CC1]

To a stirred solution of benzyl 4-[[1,2,4]triazolo[3,2-b][1,3]thiazol-2-yl]piperidine-1-carboxylate (600 mg, 1.7 mmol, 1.0 equiv.) in DMF (6 mL) was added NBS (467.80 mg, 2.6 mmol, 1.5 equiv.) and AcOH (2 mg, 0.03 mmol, 0.02 equiv.) dropwise at room temperature. The resulting mixture was stirred for 16 h at 100° C. under nitrogen atmosphere, then quenched with water at room temperature. The resulting mixture was extracted with ethyl acetate (100 mL). The organic layers were combined, washed with brine (4×20 mL), dried over anhydrous Na₂SO₄, and filtered. After filtration, the filtrate was concentrated under reduced pressure to give a residue. The residue was purified by silica gel column chromatography, eluted with PE/EtOAc (3:1) to afford benzyl 4-[5-bromo-[1,2,4]triazolo[3,2-b][1,3]thiazol-2-yl]piperidine-1-carboxylate (260 mg, 35%) as a solid. LCMS (ES, m/z): 422[M+2]⁺.

### Synthesis of Intermediate B186

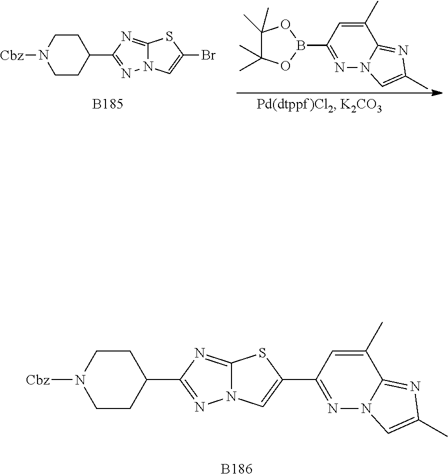

To a mixture of benzyl 4-[5-bromo-[1,2,4]triazolo[3,2-b][1,3]thiazol-2-yl]piperidine-1-carboxylate (100 mg, 0.2 mmol, 1.0 equiv.) and 2,8-dimethyl-6-(4,4,5,5-tetramethyl-1,3,2-dioxaborolan-2-yl)imidazo[1,2-b]pyridazine (97 mg, 0.3 mmol, 1.5 equiv.) in THE (2 mL) and water (0.5 mL) was added K₂CO₃ (98 mg, 0.7 mmol, 3.0 equiv.), Dppf (6 mg, 0.012 mmol, 0.05 equiv.) and Pd(DtBPF)Cl₂ (7 mg, 0.012 mmol, 0.05 equiv.). The reaction mixture was stirred for 3 h at 70° C. under a nitrogen atmosphere, then extracted with ethyl acetate (30 mL). The organic layers were combined, washed with brine (3×10 mL), dried over anhydrous Na₂SO₄, and filtered. After filtration, the filtrate was concentrated under reduced pressure. the resulting mixture was concentrated under reduced pressure to give a residue. The residue was purified by silica gel column chromatography, eluted with PE/EtOAc (1:1) to afford benzyl 4-(5-[2,8-dimethylimidazo[1,2-b]pyridazin-6-yl]-[1,2,4]triazolo[3,2-b][1,3]thiazol-2-yl)piperidine-1-carboxylate (40 mg, 34%) as a solid. LCMS (ES, m/z): 488 [M+H]⁺.

### Synthesis of Compound 114

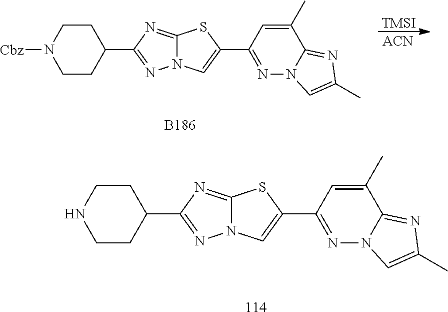

To a stirred solution of benzyl 4-(5-[2,8-dimethylimidazo[1,2-b]pyridazin-6-yl]-[1,2,4]triazolo[3,2-b][1,3]thiazol-2-yl)piperidine-1-carboxylate (40 mg, 0.08 mmol, 1.0 equiv.) in acetonitrile (2 mL) was added TMSI (24 mg, 0.12 mmol, 1.5 equiv.) dropwise at room temperature. The resulting mixture was stirred for 15 min at 70° C., then quenched with methanol at room temperature, and concentrated under vacuum to give a residue. The residue was purified by silica gel column chromatography, eluted with CH₂Cl₂/MeOH (30:1) to afford 4-(5-[2,8-dimethylimidazo[1,2-b]pyridazin-6-yl]-[1,2,4]triazolo[3,2-b][1,3]thiazol-2-yl)piperidine(2.1 mg,7%) as a solid. LCMS (ES, m/z): 354 [M+H]⁺. ¹H NMR (400 MHz, Methanol-d₄, ppm) δ 8.91 (s, 1H), 7.93 (d, J=1.0 Hz, 1H), 7.66 (d, J=1.4 Hz, 1H), 3.32-3.01 (m, 5H), 2.69 (d, J=1.1 Hz, 3H), 2.51 (d, J=0.9 Hz, 3H), 2.24 (m, 2H), 2.01 (m, 2H).

### Example 88: Synthesis of Compound 191

### Synthesis of Intermediate B187

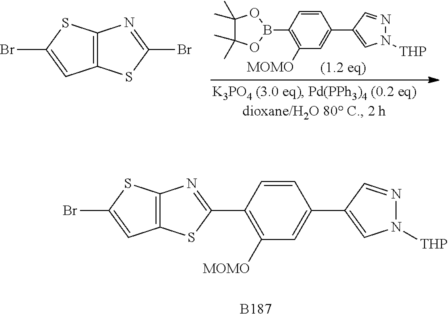

To a stirred mixture of 2,5-dibromothieno[2,3-d][1,3]thiazole (300.0 mg, 1.01 mmol, 1.00 equiv) and 4-[3-(metho-xy-methoxy)-4-(4,4,5,5-tetramethyl-1,3,2-dioxaborolan-2-yl)phenyl]-1-(oxan-2-yl)pyrazole (501.8 mg, 1.21 mmol, 1.2 equiv) in 1,4-dioxanedioxane (10 mL) and water (2 mL) was added Pd(PPh₃)₄(233.3 mg, 0.20 mmol, 0.2 equiv) and K₃PO₄ (642.4 mg, 3.03 mmol, 3.0 equiv). The reaction mixture was stirred for 2 h at 80° C. under N₂ atmosphere, then cooled to 25° C., and quenched with water (30 mL). The resulting mixture was extracted with ethyl acetate (2×50 mL). The organic layers were combined, washed with saturated aqueous NaCl (1×100 mL), dried over anhydrous Na₂SO₄, and filtered. After filtration, the filtrate was concentrated under reduced pressure to give a residue. The residue was purified by reverse flash chromatography (Condition 1, Gradient 2) to afford 4-(4-{5-bromothieno [2,3-d][1,3]thiazol-2-yl}-3-(methoxymethoxy)phenyl)-1-(oxan-2-yl)pyrazole (90.0 mg, 17.6%) as a solid. LCMS (ES, m/z): 506 [M+H]⁺.

### Synthesis of Intermediate B188

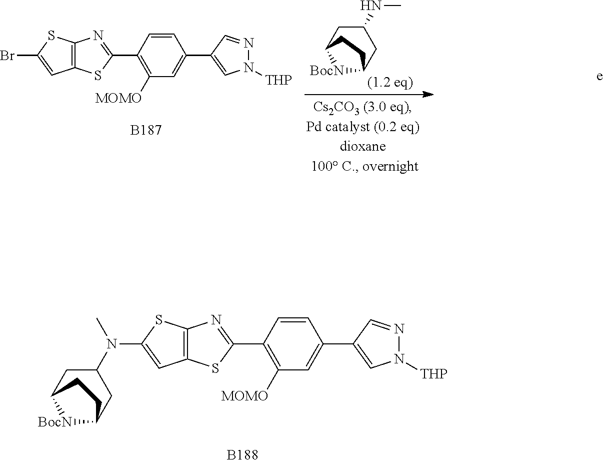

To a stirred mixture of 4-(4-{5-bromothieno[2,3-d][1,3]thiazol-2-yl}-3-(methoxymethoxy)phenyl)-1-(oxan-2-yl)-pyrazole (90.0 mg, 0.17 mmol, 1.00 equiv) and tert-butyl (1R,3R,5S)-3-(methylamino)-8-azabicyclo[3.2.1]octane-8-carboxylate (51.3 mg, 0.21 mmol, 1.2 equiv) in toluene (10 mL) was added Pd-PEPPSI-IPentCl₂-methylpyridine (opicoline)(33.5 mg, 0.03 mmol, 0.2 equiv) and Cs₂CO₃ (173.7 mg, 0.53 mmol, 3.0 equiv). The reaction mixture was stirred for 16 h at 100° C. under N₂ atmosphere, then cooled to 25° C. and quenched with water (30 mL). The resulting mixture was extracted with ethyl acetate (2×50 mL). The organic layers were combined, washed with saturated aqueous NaCl (1×100 mL), dried over anhydrous Na₂SO₄, and filtered. After filtration, the filtrate was concentrated under reduced pressure to give a residue. The residue was purified by Prep-HPLC (Condition 1) to afford tert-butyl (1R,3R,5S)-3-({2-[2-(methoxymethoxy)-4-[1-(oxan-2-yl) pyrazol-4-yl]-phenyl]-thieno[2,3-d] [1,3]thiazol-5-yl}(methyl)amino)-8-azabicyclo[3.2.1] octane-8-carboxylate (45.0 mg, 38.1%) as a solid. LCMS (ES, m/z): 666 [M+H]⁺.

### Synthesis of Compound 191

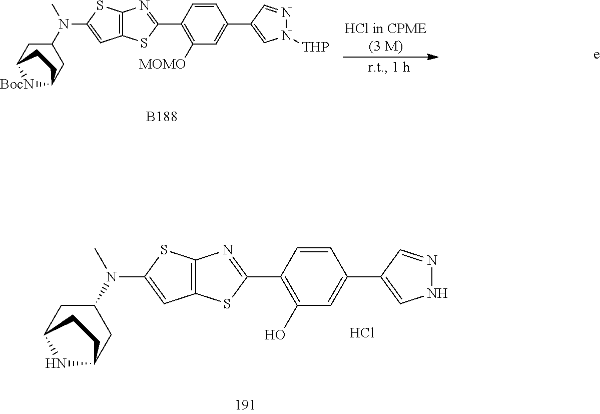

A mixture of tert-butyl (1R,3R,5S)-3-({2-[2-(methoxymethoxy)-4-[1-(oxan-2-yl) pyrazol-4-yl]phenyl]thieno[2,3-d][1,3]thiazol-5-yl}(methyl)amino)-8-azabicyclo[3.2.1]octane-8-carboxylate (20.0 mg, 0.03 mmol, 1.00 equiv) and 3 M HCl in CPME (0.5 mL) was stirred for 1 h at room temperature. A precipitate formed that was collected and was purified by Prep-HPLC (Condition 7, Gradient 6) to afford 2-{5-[(1R,3R,5S)-8-azabicyclo[3.2.1]octan-3-yl(methyl)amino]thieno[2,3-d][1,3]-thiazol-2-yl}-5-(1H-pyrazol-4-yl)phenol (5.9 mg, 42.06%) as a solid. LCMS (ES, m/z): 438 [M+H]⁺. ¹H-NMR (400 MHz, DMSO-d₆) δ 11.06 (s, 1H), 9.10 (s, 1H), 8.87 (s, 1H), 8.07 (s, 2H), 8.04-7.95 (m, 1H), 7.21 (td, J=4.3, 1.7 Hz, 2H), 6.47 (s, 1H), 4.08 (s, 2H), 3.86 (m, 1H), 2.84 (s, 3H), 2.17 (t, J=12.7 Hz, 2H), 2.02 (s, 4H), 1.84 (d, J=13.4 Hz, 2H).

### Example 89: Synthesis of Compounds 159 and 160

### Synthesis of Intermediate B189

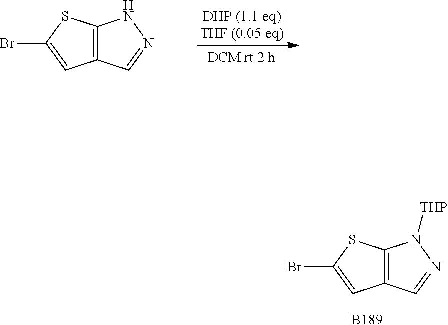

A solution of 5-bromo-1H-thieno[2,3-c] pyrazole (2.0 g, 9.36 mmol, 1.0 equiv) in DCM (20.0 ml) was treated with DHP (0.91 g, 10.29 mmol, 1.1 equiv). The reaction mixture was stirred for 5 minutes at 25° C. under nitrogen atmosphere, then TFA (0.06 g, 0.47 mmol, 0.05 equiv) was added dropwise at 25° C. The reaction mixture was stirring for an additional 2 h at 25° C. The resulting mixture was extracted with ethyl acetate (2×50.0 mL). The organic layers were combined, washed with saturated brine (50.0 mL), dried over anhydrous Na₂SO₄, and filtered. After filtration, the filtrate was concentrated under reduced pressure to afford a solid (2.80 g). LCMS (ES, m/z): 287 [M+H]⁺

### Synthesis of Intermediate B190

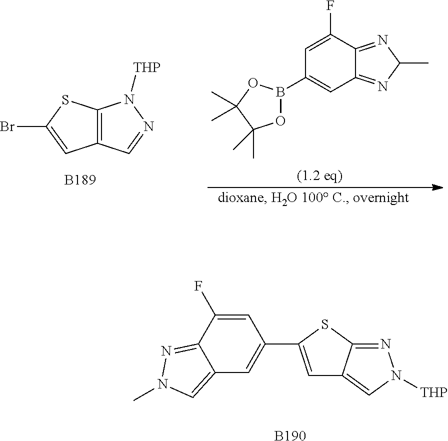

To a stirred mixture of 5-bromo-1-(oxan-2-yl) thieno[2,3-c] pyrazole (1.90 g, 6.62 mmol, 1.00 equiv) and 7-fluoro-2-methyl-5-(4,4,5,5-tetramethyl-1,3,2-dioxaborolan-2-yl) indazole (2.37 g, 8.60 mmol, 1.30 equiv) in 1,4-dioxane (19.00 mL) and water (3.80 mL) was added Pd(dtbpf)Cl₂ (0.43 g, 0.66 mmol, 0.10 equiv) and K₃PO₄ (4.21 g, 19.85 mmol, 3.00 equiv) under N₂ atmosphere. The reaction mixture was stirred overnight at 100° C. The resulting mixture was extracted with ethyl acetate (2×50.00 mL). The organic layers were combined, washed with saturated brine solution (50.00 mL), dried over anhydrous Na₂SO₄, and filtered. After filtration, the filtrate was concentrated under reduced pressure to give a residue. The residue was purified by silica gel column chromatography, eluted with EA and PE (5:2) to afford 1.8 g 7-fluoro-2-methyl-5-[2-(oxan-2-yl) thieno[2,3-c] pyrazol-5-yl] indazole as a solid. LCMS (ES, m/z): 357 [M+H]⁺

### Synthesis of Intermediate B191

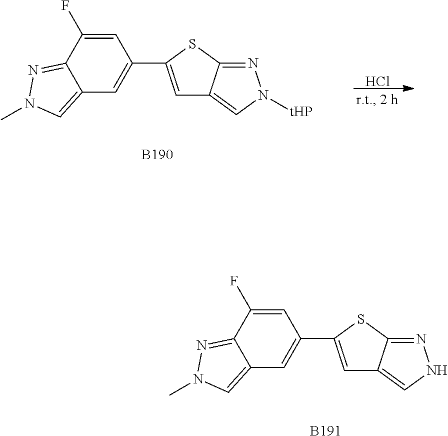

A solution of 7-fluoro-2-methyl-5-[2-(oxan-2-yl) thieno[2,3-c] pyrazol-5-yl] indazole (1.80 g) in HCl (gas) in 1,4-dioxane (18.0 mL) and 1,4-dioxane (18.0 mL) was stirred for 2 h at 25° C. under N₂ atmosphere. The mixture was concentrated under reduced pressure to afford 7-fluoro-2-methyl-5-{2H-thieno[2,3-c] pyrazol-5-yl} indazole (2 g) as a solid. LCMS (ES, m/z): 273 [M+H]⁺.

### Synthesis of Intermediate B192 and B193

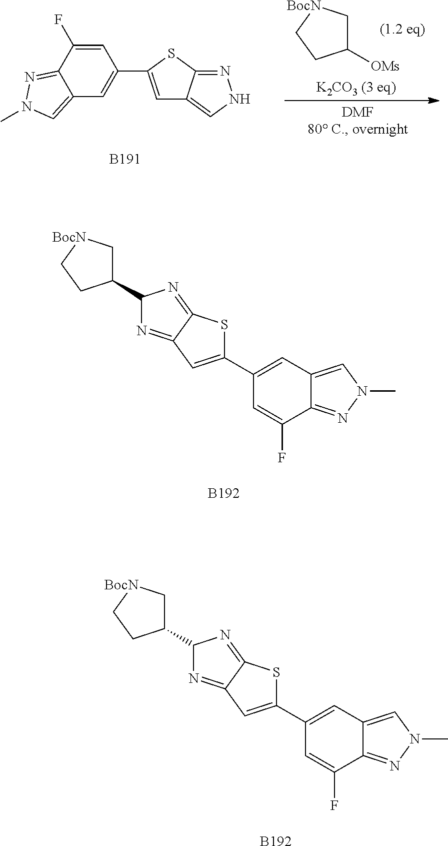

To a stirred mixture of 7-fluoro-2-methyl-5-{2H-thieno[2,3-c] pyrazol-5-yl} indazole (200.0 mg, 0.73 mmol, 1.00 equiv) and tert-butyl 3-(methanesulfonyloxy) pyrrolidine-1-carboxylate (233.9 mg, 0.88 mmol, 1.20 equiv) in DMF (4.00 mL, 51.6 mmol, 70.37 equiv) was added K₂CO₃ (304.5 mg, 2.20 mmol, 3 equiv) at 25° C. under N₂ atmosphere. The reaction mixture was stirred overnight at 80° C., then concentrated under reduced pressure to give a residue. The residue was purified by silica gel column chromatography, eluted with EA and PE (2:5), followed by chiral-prep-HPLC (Condition 6, Gradient 1) to afford tert-butyl (3S)-3-[5-(7-fluoro-2-methylindazol-5-yl)thieno[2,3-c]pyrazol-2-yl] pyrrolidine-1-carboxylate (30 mg, 9.25%) and tert-butyl (3R)-3-[5-(7-fluoro-2-methylindazol-5-yl) thieno[2,3-c] pyrazol-2-yl]pyrrolidine-1-carboxylate (50 mg, 15.4%) as solids.

### Synthesis of Compound 159

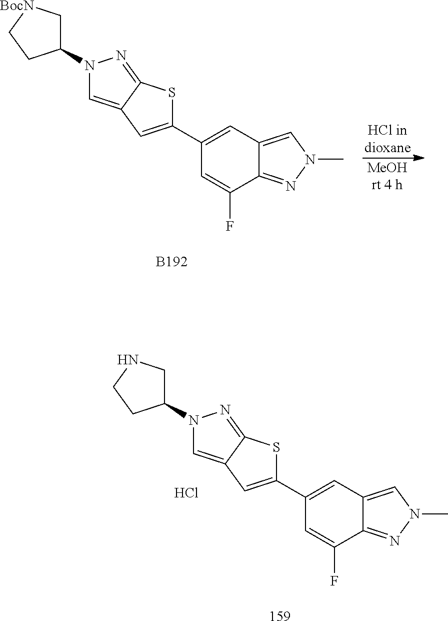 [SMILES: "B192" ; Cn1cc2cc(-c3cc4cn([C@H]5CCN(C(=O)OC(C)(C)C)C5)nc4s3)cc(F)c2n1 ; Cn1cc2cc(-c3cc4cn([C@H]5CCNC5)nc4s3)cc(F)c2n1 ; Cl ; "159"]

To a stirred solution of tert-butyl 3-[5-(7-fluoro-2-methylindazol-5-yl) thieno[2,3-c] pyrazol-2-yl] pyrrolidine-1-carboxylate (30.0 mg) in methanol was added HCl (gas) in 1,4-dioxane at 25° C. under N₂ atmosphere. The reaction was stirred for 4 h at 25° C., then concentrated under reduced pressure to afford 7-fluoro-2-methyl-5-{2-[(3S)-pyrrolidin-3-yl] thieno [2,3-c] pyrazol-5-yl} indazole hydrochloride (2.0 mg) as a solid. LCMS (ES, m/z): 342 [M+H]⁺. ¹H NMR (400 MHz, DMSO-d₆) δ 9.67 (s, 1H), 9.39 (s, 1H), 8.51 (d, J=2.8 Hz, 1H), 8.26 (s, 1H), 7.73 (d, J=1.3 Hz, 1H), 7.53 (s, 2H), 5.36 (tt, J=7.3, 3 Hz, 1H), 4.21 (s, 3H), 3.75-3.57 (m, 2H), 3.49 (d, J=6.1 Hz, 1H), 3.38 (dd, J=8.1, 4.6 Hz, 1H), 2.41-2.29 (m, 2H).

### Synthesis of Compound 160

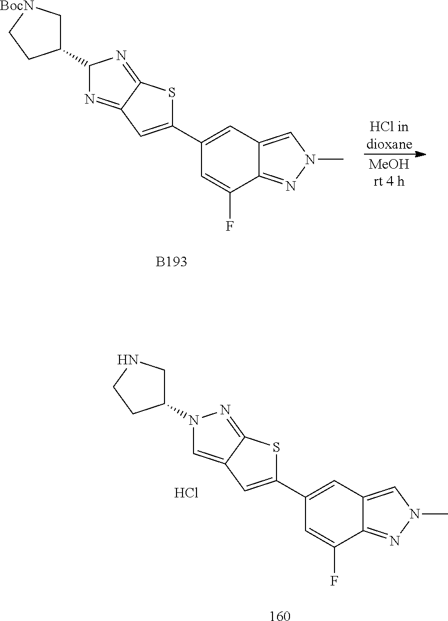 [SMILES: "B193" ; Cn1cc2cc(-c3cc4c(s3)=NC([C@@H]3CCN(C(=O)OC(C)(C)C)C3)N=4)cc(F)c2n1 ; Cn1cc2cc(-c3cc4cn([C@@H]5CCNC5)nc4s3)cc(F)c2n1 ; Cl ; "160"]

To a stirred solution of tert-butyl 3-[5-(7-fluoro-2-methylindazol-5-yl) thieno[2,3-c] pyrazol-2-yl] pyrrolidine-1-carboxylate (50.0 mg, 0.11 mmol, 1.00 equiv) in methanol (2.00 mL) was added HCl (gas) in 1,4-dioxane (0.50 mL) at 25° C. under N₂ atmosphere. The reaction mixture was stirred for 4 h at 25° C., then concentrated under reduced pressure to afford (R)-5-(7-fluoro-2-methyl-2H-indazol-5-yl)-2-(pyrrolidin-3-yl)-2H-thieno[2,3-c]pyrazole hydrochloride (7.7 mg) as a solid. LCMS (ES, m/z): 342 [M+H]⁺. ¹H NMR (400 MHz, DMSO-d₆) δ 9.89 (s, 1H), 9.53 (s, 1H), 8.51 (d, J=2.7 Hz, 1H), 8.27 (s, 1H), 7.73 (d, J=1.3 Hz, 1H), 7.52 (s, 2H), 5.36 (tt, J=7.4, 3.9 Hz, 1H), 4.21 (s, 3H), 3.69 (tt, J=10.8, 5.3 Hz, 1H), 3.60 (ddd, J=11.6, 7.0, 3.6 Hz, 1H), 3.48 (dp, J=13.9, 6.9 Hz, 1H), 3.38 (qd, J=10.8, 5.3 Hz, 1H), 2.46 (t, J=7.0 Hz, 1H), 2.38-2.28 (m, 1H).

### Example 90: Synthesis of Compound 161

### Synthesis of Compound 161

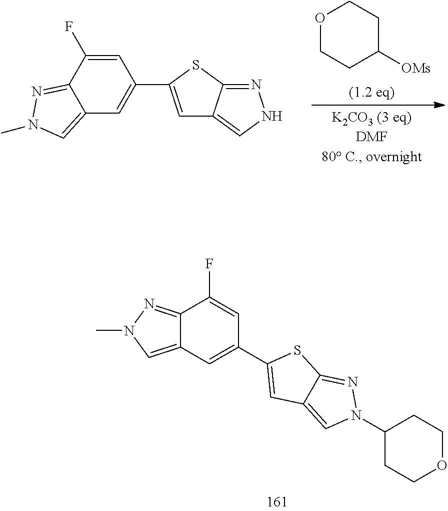 [SMILES: Cn1cc2cc(-c3cc4c[nH]nc4s3)cc(F)c2n1 ; CS(=O)(=O)OC1CCOCC1 ; Cn1cc2cc(-c3cc4cn(C5CCOCC5)nc4s3)cc(F)c2n1]

To a stirred mixture of 7-fluoro-2-methyl-5-{2H-thieno[2,3-c] pyrazol-5-yl} indazole (200.0 mg, 0.73 mmol, 1.00 equiv) and oxan-4-yl methanesulfonate (158.8 mg, 0.88 mmol, 1.20 equiv) in DMF (4.00 mL, 51.69 mmol, 70.37 equiv) was added K₂CO₃ (304.5 mg, 2.20 mmol, 3.00 equiv) under nitrogen atmosphere. The reaction mixture was stirred overnight at 80° C., then concentrated under reduced pressure to give a residue. The residue was purified by silica gel column chromatography, eluted with PE/EA (1:1), followed by chiral-prep-HPLC (Condition 7, Gradient 1) to afford 7-fluoro-2-methyl-5-[2-(oxan-4-yl) thieno[2,3-c] pyrazol-5-yl] indazole (17.8 mg, 6.8%) as a solid. LCMS (ES, m/z): 357 [M+H]⁺. ¹H NMR (400 MHz, DMSO-d₆) δ 8.49 (d, J=2.8 Hz, 1H), 8.15 (s, 1H), 7.71 (d, J=1.4 Hz, 1H), 7.53 (dd, J=13.1, 1.5 Hz, 1H), 7.48 (s, 1H), 4.64-4.51 (m, 1H), 4.21 (s, 3H), 4.00 (dt, J=11.7, 3.5 Hz, 2H), 3.57-3.42 (m, 2H), 2.05 (td, J=9.0, 8.0, 4.2 Hz, 4H).

### Example 91: Synthesis of Compound 162

### Synthesis of Intermediate B194

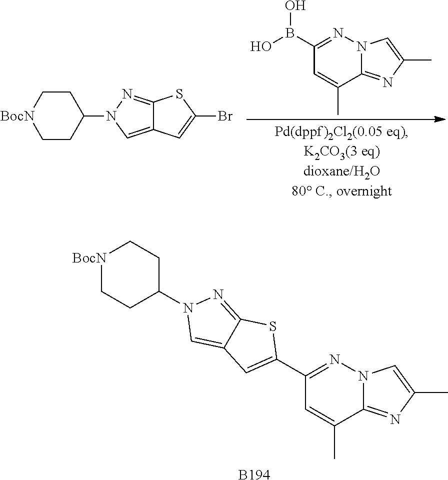 [SMILES: CC(C)(C)OC(=O)N1CCC(n2cc3cc(Br)sc3n2)CC1 ; Cc1cn2nc(B(O)O)cc(C)c2n1 ; "B194" ; Cc1cn2nc(-c3cc4cn(C5CCN(C(=O)OC(C)(C)C)CC5)nc4s3)cc(C)c2n1]

To a stirred mixture of tert-butyl 4-{5-bromothieno[2,3-c]pyrazol-2-yl}piperidine-1-carboxylate (300 mg, 0.777 mmol, 1.00 equiv) and 2,8-dimethylimidazo[1,2-b]pyridazin-6-ylboronic acid (296.65 mg, 1.554 mmol, 2 equiv) in dioxane (3 mL, 35.412 mmol, 45.60 equiv) was added Pd(dppf)Cl₂ (31.63 mg, 0.039 mmol, 0.05 equiv), K₂CO₃ (321.98 mg, 2.331 mmol, 3 equiv) and water (0.5 mL, 27.754 mmol, 35.74 equiv) in portions at room temperature under nitrogen atmosphere. The resulting mixture was stirred overnight at 80° C. under nitrogen atmosphere. The reaction mixture was filtered and the filtrate was concentrated under reduced pressure to give a residue. The residue was purified by Chiral-Prep-HPLC (Condition 8, Gradient 1) to afford tert-butyl4-(5-{2,8-dimethylimidazo[1,2-b]pyridazin-6-yl}thieno[2,3-c]pyrazol-2-yl)piperidine-1-carboxylate (100 mg, 28.45%) as a solid.

### Synthesis of Compound 162

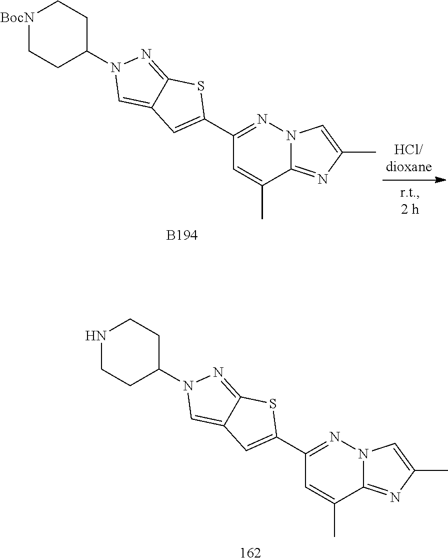 [SMILES: "B194" ; Cc1cn2nc(-c3cc4cn(C5CCN(C(=O)OC(C)(C)C)CC5)nc4s3)cc(C)c2n1 ; Cc1cn2nc(-c3cc4cn(C5CCNCC5)nc4s3)cc(C)c2n1 ; "162"]

To a stirred solution of tert-butyl 4-(5-{2,8-dimethylimidazo[1,2-b]pyridazin-6-yl}thieno[2,3-c]pyrazol-2-yl)piperidine-1-carboxylate (100 mg, 0.221 mmol, 1.00 equiv) in methanol (2 mL, 49.398 mmol, 223.56 equiv) was added HCl (gas) in 1,4-dioxane (2 mL, 65.824 mmol, 297.91 equiv) in portions at room temperature under nitrogen atmosphere. The resulting mixture was stirred for 1 h at room temperature under nitrogen atmosphere, then filtered. After filtration, the filtrate was concentrated under reduced pressure to give a residue. The residue was purified by Prep-HPLC (Condition 2, Gradient 5) to afford 4-(5-{2,8-dimethylimidazo[1,2-b]pyridazin-6-yl}thieno[2,3-c]pyrazol-2-yl)piperidine (20.6 mg, 26.45%) as a solid. LCMS (ES, m/z): 353 [M+H]⁺. 1H NMR (400 MHz, DMSO-d₆) δ 8.24 (s, 1H), 7.99 (s, 1H), 7.88 (s, 1H), 7.71 (s, 1H), 4.37 (t, J=11.3 Hz, 1H), 3.10-3.02 (m, 2H), 2.62 (s, 2H), 2.51 (s, 3H), 2.37 (s, 3H), 2.01 (d, J=11.8 Hz, 2H), 1.88 (q, J=12.1 Hz, 2H).

### Example 92: Synthesis of Compounds 195 and 196

### Synthesis of Intermediate B195

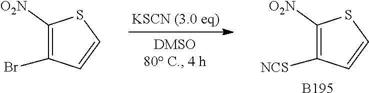

To a stirred mixture of 3-bromo-2-nitrothiophene (140.0 g, 672.98 mmol, 1.00 equiv) and KSCN (196.8 g, 2028.87 mmol, 3.01 equiv) in DMSO (450.00 mL) was stirred for 4 h at 80° C. The resulting mixture was extracted with EtOAc (3×500 mL). The combined organic layers were washed with sat. NaCl (3×500 mL), dried over anhydrous Na₂SO₄. After filtration, the filtrate was concentrated under reduced pressure. This resulted in [(2-nitrothiophen-3-yl)sulfanyl]formonitrile (120.0 g, 95.7%) as a solid. LCMS (ES, m/z):187 [M+H]⁺.

### Synthesis of Intermediate B196

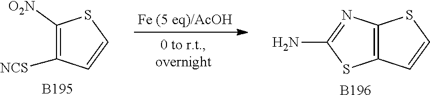

A mixture of [(2-nitrothiophen-3-yl)sulfanyl]formonitrile (120.0 g, 644.47 mmol, 1.00 equiv) and Fe (179.9 g, 3222.34 mmol, 5.00 equiv) in AcOH (2.50 L) was stirred for 8 h at room temperature. The resulting mixture was filtered, the filter cake washed with methanol (1×2 L), and the filtrate concentrated under reduced pressure to give a residue. The residue was purified by silica gel column chromatography, eluted with PE/EA (10:01) to afford 2-amino-5-methyl-1,3-thiazole-4-thiol (50.0 g, 53.0%) as a solid. LCMS (ES, m/z):157 [M+H]⁺.

### Synthesis of Intermediate B197

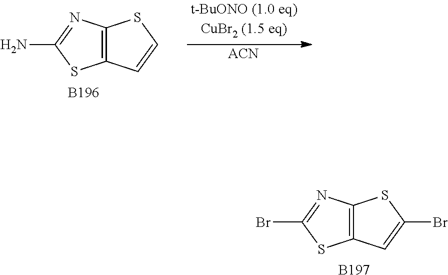 [SMILES: "B196" ; "B197" ; Nc1nc2sccc2s1 ; "ACN" ; Brc1cc2sc(Br)nc2s1]

To a stirred mixture of CuBr₂ (21.5 g, 96.02 mmol, 1.50 equiv) and t-BuONO (6.6 g, 64.01 mmol, 1.00 equiv) in acetonitrile (300 mL) was added thieno[2,3-d][1,3]thiazol-2-amine (10.0 g, 64.01 mmol, 1.00 equiv) in portions at 65° C. The resulting mixture was extracted with ethyl acetate (3×200 mL). The organic layers were combined, washed with saturated NaCl (1×200 mL), dried over anhydrous Na₂SO₄, and filtered. After filtration, the filtrate was concentrated under reduced pressure to give a residue. The residue was purified by silica gel column chromatography, eluted with PE/EA (12:01) to afford 2,5-dibromothieno[2,3-d][1,3]thiazole (4.3 g, 22.4%) as a solid. LCMS (ES, m/z):298 [M+H]⁺.

### Synthesis of Intermediate B198

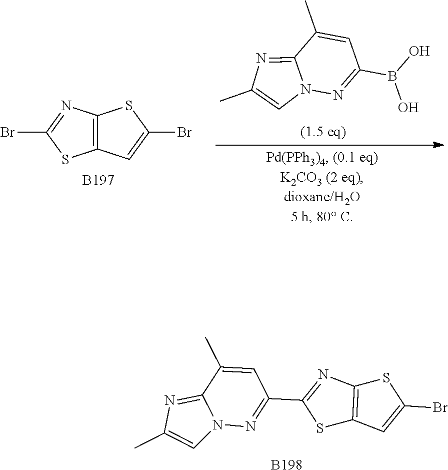 [SMILES: "B197" ; "B198" ; Brc1cc2sc(Br)nc2s1 ; Cc1cn2nc(-c3nc4sc(Br)cc4s3)cc(C)c2n1 ; Cc1cn2nc(B(O)O)cc(C)c2n1]

To a stirred mixture of 2,5-dibromothieno[2,3-d][1,3]thiazole (3.0 g, 10.03 mmol, 1.00 equiv) and 2,8-dimethyl-6-(4,4,5,5-tetramethyl-1,3,2-dioxaborolan-2-yl)imidazo[1,2-b]pyridazine (4.1 g, 15.05 mmol, 1.50 equiv) in dioxane/water (30 mL) was added Pd(PPh₃)₄(1.2 g, 1.00 mmol, 0.10 equiv) and K₂CO₃ (2.8 g, 20.07 mmol, 2.00 equiv). The reaction mixture was stirred for 4 h at 80° C. under nitrogen atmosphere, then extracted with ethyl acetate (3×30 mL). The organic layers were combined, washed with saturated NaCl (1×30 mL), dried over anhydrous Na₂SO₄, and filtered. After filtration, the filtrate was concentrated under reduced pressure to give a residue. The residue was purified by silica gel column chromatography, eluted with PE/EA (5:01) to afford 6-{5-bromothieno[2,3-d][1,3]thiazol-2-yl}-2,8-dimethylimidazo[1,2-b]pyridazine (500.0 mg, 13.6%) as a solid. LCMS (ES, m/z):365 [M+H]⁺.

### Synthesis of Compound 195

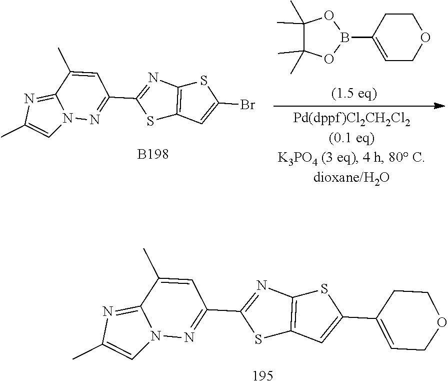

To a stirred mixture of 6-{5-bromothieno[2,3-d][1,3]thiazol-2-yl}-2,8-dimethylimidazo [1,2-b]pyridazine (200.00 mg, 0.55 mmol, 1.00 equiv) and 2-(3,6-dihydro-2H-pyran-4-yl)-4,4,5,5-tetramethyl-1,3,2-dioxaborolane (172.54 mg, 0.82 mmol, 1.50 equiv) in dioxane/water (5 mL) was added Pd(dppf)Cl₂·CH₂Cl₂ (44.60 mg, 0.06 mmol, 0.10 equiv) and K₃PO₄ (348.67 mg, 1.64 mmol, 3.00 equiv). The reaction mixture was stirred for 8 h at 80° C. under nitrogen atmosphere, then extracted with ethyl acetate (3×10 mL). The organic layers were combined, washed with saturated NaCl (1×10 mL), dried over anhydrous Na₂SO₄, and filtered. After filtration, the filtrate was concentrated under reduced pressure to give a residue. The residue was purified by silica gel column chromatography, eluted with PE/EA (1:01) to afford 6-[5-(3,6-dihydro-2H-pyran-4-yl)thieno[2,3-d][1,3] thiazol-2-yl]-2,8-dimethylimidazo[1,2-b]pyridazine (83.00 mg, 41.14%) as a solid. LCMS (ES, m/z):369 [M+H]⁺. ¹H-NMR (400 MHz, DMSO-d₆) δ 8.15 (s, 1H), 7.83 (s, 1H), 7.55 (s, 1H), 6.33 (s, 1H), 4.25 (d, J=3.2 Hz, 2H), 3.85 (t, J=5.4 Hz, 2H), 2.63 (s, 3H), 2.51 (s, 2H) 2.42 (s, 3H).

### Synthesis of Compound 196

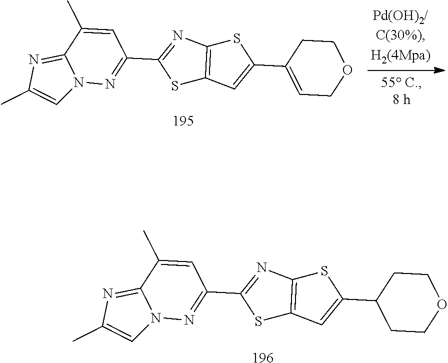

A mixture of 6-[5-(3,6-dihydro-2H-pyran-4-yl)thieno[2,3-d][1,3]thiazol-2-yl]-2,8-dimethyl-limidazo[1,2-b]pyridazine (53.0 mg, 0.14 mmol, 1.00 equiv) and Pd(OH)₂/C (15.00 mg, 0.11 mmol, 0.74 equiv) in THE (5 mL) was stirred for 8 h at 55° C. under hydrogen (4 MPa) atmosphere. The resulting mixture was filtered, the filter cake was washed with THE (3×5 mL), and the filtrate concentrated under reduced pressure to give a residue. The resulting solid was washed with methanol (3×2 mL) and dried to afford 2,8-dimethyl-6-[5-(oxan-4-yl)thieno[2,3-d][1,3]thiazol-2-yl]imidazo[1,2-b]pyridazine (10.4 mg, 19.5%) as a solid. LCMS (ES, m/z):371 [M+H]⁺. 1H NMR (400 MHz, DMSO-d₆) δ 8.15 (d, J=1.0 Hz, 1H), 7.84 (q, J=1.1 Hz, 1H), 7.39 (d, J=1.0 Hz, 1H), 4.00-3.92 (m, 2H), 3.48 (td, J=11.7, 2.0 Hz, 2H), 3.27-3.18 (m, 1H), 2.64 (d, J=1.1 Hz, 3H), 2.48-2.40 (m, 3H), 1.97 (d, J=13.3 Hz, 2H), 1.72 (qd, J=12.2, 4.3 Hz, 2H)

### Example 93: Exemplary Splicing Assay for Monitoring Expression Levels of Splice Variants

Compounds described herein were used to modulate RNA transcript abundance in cells. The expression of a target mRNA was measured by detecting the formation of an exon-exon junction in the canonical transcript (CJ). A compound mediated exon-inclusion event was detected by observing an increase in formation of a new junction with an alternative exon (AJ). Real-time qPCR assays were used to detect these splicing switches and interrogate the potency of various compounds towards different target genes. A high-throughput real time quantitative PCR (RT-qPCR) assay was developed to measure these two isoforms of the mRNA (CJ and AJ) for an exemplary gene, HTT, together with a control housekeeping gene, GAPDH or GUSB or PPIA, used for normalization. Briefly, the A673 or K562 cell line was treated with various compounds described herein (e.g., compounds of Formula (I)). After treatment, the levels of the HTT mRNA targets were determined from each sample of cell lysate by cDNA synthesis followed by qPCR.

### Materials:

Cells-to-C_{T} 1-step kit: ThermoFisher A25602, Cells-to-C_{T} lysis reagent: ThermoFisher 4391851C, TaqMan™ Fast Virus 1-Step Master Mix: ThermoFisher 4444436
GAPDH: VIC-PL, ThermoFisher 4326317E (Assay: Hs99999905_ml)—used for K562/suspension cell lines
GUSB: VIC-PL, ThermoFisher 4326320E (Assay: Hs99999908_ml)—used for K562/suspension cell lines
PPIA: VIC-PL, ThermoFisher 4326316E (Assay: Hs99999904_ml)—used for A673/adherent cell lines

### Probe/Primer Sequences

Canonical junction (CJ)
HTT Primer 1:
TCCTCCTGAGAAAGAGAAGGAC
HTT Primer 2:
GCCTGGAGATCCAGACTCA
HTT CY5-Probe:
/5Cy5/TGGCAACCCTTGAGGCCCTGTCCT/3IAbRQSp/
Alternative junction (AJ)
HTT Primer 1:
TCCTGAGAAAGAGAAGGACATTG
HTT Primer 2:
CTGTGGGCTCCTGTAGAAATC
HTT FAM-Probe:
/56-FAM/TGGCAACCC/ZEN/TTGAGAGGCAAGCCCT/3IABKFQ/

### Description

The A673 cell line was cultured in DMEM with 10% FBS. Cells were diluted with full growth media and plated in a 96-well plate (15,000 cells in 100ul media per well). The plate was incubated at 37° C. with 5% CO₂ for 24 hours to allow cells to adhere. An 11-point 3-fold serial dilution of the compounds was made in DMSO then diluted in media in an intermediate plate. Compounds were transferred from the intermediate plate to the cell plate with the top dose at a final concentration of 10 uM in the well. Final DMSO concentration was kept at or below 0.25%. The cell plate was returned to the incubator at 37° C. with 5% CO₂ for an additional 24 hours.

The K562 cell line was cultured in IMDM with 10% FBS. For K562, cells were diluted with full growth media and plated in either a 96-well plate (50,000 cells in 50 uL media per well) or a 384-well plate (8,000-40,000 cells in 45 uL media per well). An 11-point 3-fold serial dilution of the compounds were made in DMSO then diluted in media in an intermediate plate. Compound was transferred from the intermediate plate to the cell plate with the top dose at a final concentration of 10 uM in the well. Final DMSO concentration was kept at or below 0.25%. Final volume was 100 uL for 96-well plate and 50 uL for 384-well plate. The cell plate was then placed in an incubator at 37° C. with 5% CO2 for 24 hours.

The cells were then gently washed with 50 uL-100 uL cold PBS before proceeding to addition of lysis buffer. 30 uL-50 uL of room temperature lysis buffer with DNAse I (and optionally RNAsin) was added to each well. Cells were shaken/mixed thoroughly at room temperature for 5-10 minutes for lysis to take place and then 3 uL-5 uL of room temperature stop solution was added and wells were shaken/mixed again. After 2-5 minutes, the cell lysate plate was transferred to ice for RT-qPCR reaction setup. The lysates could also be frozen at −80° C. for later use.

In some cases, a direct lysis buffer was used. An appropriate volume of 3X lysis buffer (10 mM Tris, 150 mM NaCl, 1.5%-2.5% Igepal and 0.1-1 U/uL RNAsin, pH 7.4) was directly added to either K562 or A673 cells in media and mixed by pipetting 3 times. The plates were then incubated at room temperature with shaking/rocking for 20-50 minutes to allow for lysis to take place. After this time, the cell lysate plate was transferred to ice to set up for the RT-qPCR reactions. The lysates could also be frozen at −80° C. for later use.

To set up 10 uL RT-qPCR reactions, cell lysates were transferred to 384-well qPCR plates containing the master mix according to the table below. The plates were sealed, gently vortexed, and spun down before the run. The volumes were adjusted accordingly in some instances where the reaction was carried in 20 uL. The table below summarizes the components of the RT-qPCR reactions:

| Component | 1× |
|---|---|
| Taqman 1-step RT-qPCR mix (4×) | 2.5 |
| 20× AJ Primers + Probe (FAM) | 0.5 |
| 20× CJ Primers + Probe (CY5) | 0.5 |
| 20× PPIA Control (VIC) | 0.5 |
| Cell lysate (1×) | 1-2 |
| H₂O | 4-5 |
| Total volume | 10 |

The RT-qPCR reaction was performed using a QuantStudio (ThermoFisher) under the following fast cycling conditions. All samples and standards were analyzed at least in duplicate. In some instances, bulk room temperature (RT) step of 5-10 minutes was completed for all plates before proceeding with qPCR. The table below summarizes the PCR cycle:

| Step | # cycles | Temp. | Time | |
|---|---|---|---|---|
| RT step | 1 | 50° C. | 5 | min |
| RT inactivation/initial | 1 | 95° C. | 20 | sec |
| denaturation | | | | |
| Amplification | 40 | 95° C. | 3 | sec |
| | | 60° C. | 30 | sec |

The data analysis was performed by first determining the ΔCt vs the housekeeper gene. This ΔCt was then normalized against the DMSO control (ΔΔCt) and converted to RQ (relative quantification) using the 2\hat{ }(−ΔΔCt) equation. The RQ were then converted to a percentage response by arbitrarily setting an assay window of 3.5 ΔCt for HTT-CJ and an assay window of 9 ΔCt for HTT-AJ. These assay windows correspond to the maximal modulation observed at high concentration of the most active compounds. The percentage response was then fitted to the 4 parametric logistic equation to evaluate the concentration dependence of compound treatment. The increase in AJ mRNA is reported as AC₅₀ (compound concentration having 50% response in AJ increase) while the decrease in CJ mRNA levels is reported as IC₅₀ (compound concentration having 50% response in CJ decrease).

A summary of these results is illustrated in Table 5, wherein “A” represents an AC₅₀/IC₅₀ of less than 100 nM; “B” represents an AC₅₀/IC₅₀ of between 100 nM and 1 μM; and “C” represents an AC₅₀/IC₅₀ of between 1 μM and 10 μM; and “D” represents an AC₅₀/IC₅₀ of greater than 10 μM.

TABLE 5
Modulation of RNA Splicing by Exemplary Compounds

| | HTT AJ | HTT CJ |
|---|---|---|
| Compound | AC₅₀ | IC₅₀ |
| No. | (nM) | (nM) |
| 100 | C | — |
| 101 | B | A |
| 102 | B | B |
| 103 | D | D |
| 104 | B | A |
| 105 | B | B |
| 106 | A | A |
| 110 | B | B |
| 112 | A | A |
| 121 | B | B |
| 122 | B | A |
| 124 | A | B |
| 125 | B | B |
| 126 | B | B |
| 127 | C | C |
| 128 | C | C |
| 130 | A | A |
| 131 | C | C |
| 132 | A | A |
| 133 | B | B |
| 134 | A | B |
| 135 | B | B |
| 136 | D | D |
| 137 | B | B |
| 139 | A | A |
| 140 | A | A |
| 141 | B | B |
| 142 | B | B |
| 143 | C | C |
| 144 | B | C |
| 145 | D | D |
| 146 | B | B |
| 147 | B | B |
| 149 | A | A |
| 150 | B | B |
| 151 | A | A |
| 154 | B | B |
| 155 | B | B |
| 156 | B | A |
| 157 | B | B |
| 158 | B | B |
| 160 | C | C |
| 161 | D | D |
| 162 | A | A |
| 163 | C | C |
| 164 | C | C |
| 165 | A | A |
| 166 | B | B |
| 167 | B | B |
| 168 | C | C |
| 169 | C | C |
| 170 | C | C |
| 171 | B | B |
| 172 | B | B |
| 173 | C | C |
| 174 | D | D |
| 175 | D | D |
| 176 | D | D |
| 177 | D | D |
| 178 | C | C |
| 180 | D | D |
| 181 | C | C |
| 182 | B | A |
| 183 | C | B |
| 184 | C | C |
| 187 | A | A |
| 188 | C | B |
| 189 | D | D |
| 190 | B | B |
| 191 | D | D |
| 192 | A | A |
| 193 | A | A |
| 195 | B | B |
| 196 | C | C |
| 197 | B | B |
| 198 | B | A |
| 200 | D | D |
| 201 | D | D |
| 202 | D | D |
| 203 | D | D |
| 204 | C | C |
| 205 | C | C |
| 206 | A | A |
| 207 | B | B |
| 208 | B | B |
| 209 | C | B |
| 210 | A | A |
| 211 | C | C |
| 212 | C | C |
| 213 | C | C |
| 214 | B | C |
| 215 | B | B |
| 216 | A | A |
| 218 | B | A |
| 219 | D | D |
| 220 | C | C |
| 221 | D | D |
| 222 | C | C |
| 223 | C | C |
| 224 | D | D |
| 225 | A | A |
| 226 | A | A |
| 231 | C | C |
| 233 | C | C |
| 235 | C | C |
| 236 | C | B |
| 237 | D | D |
| 238 | A | A |

Additional studies were carried out for a larger panel of genes using the protocol provided above. The junction between flanking upstream and downstream exons was used to design canonical junction qPCR assays. At least one of the forward primer, reverse primer or the CY5-labeled 5′ nuclease probe (with 3′ quencher such as ZEN/Iowa Black FQ) was designed to overlap with the exon junction to capture the CJ mRNA transcript. BLAST was used to confirm the specificity of the probeset and parameters such as melting temperature, GC content, amplicon size, and primer dimer formation are considered during their design. Data for the decrease in CJ mRNA levels for three exemplary genes (HTT, SMN2, and Target C) analyzed in this panel are reported as IC₅₀ (compound concentration having 5000 response in CJ decrease).

A summary of the results from the panel is illustrated in Table 6, wherein “A” represents an IC₅₀ of less than 100 nM; “B” represents an IC₅₀ of between 100 nM and 1 μM; and “C” represents an IC₅₀ of between 1 μM and 10 μM; and “D” represents an IC₅₀ of greater than 10 μM.

TABLE 6
Modulation of RNA Splicing by Exemplary Compounds

| Compound No. | HTT | SMN2 | Target C |
|---|---|---|---|
| 100 | — | — | D |
| 101 | A | A | A |
| 102 | B | A | B |
| 103 | D | D | D |
| 104 | A | A | B |
| 105 | B | B | C |
| 106 | A | A | A |
| 110 | B | A | — |
| 112 | A | A | A |
| 121 | B | A | — |
| 122 | A | A | A |
| 124 | B | A | B |
| 125 | B | B | C |
| 126 | B | B | C |
| 127 | C | C | C |
| 128 | C | B | C |
| 130 | A | A | A |
| 131 | C | C | C |
| 132 | A | A | B |
| 133 | B | B | B |
| 134 | B | A | B |
| 135 | B | A | B |
| 136 | D | B | D |
| 137 | B | A | B |
| 139 | A | A | A |
| 140 | A | A | B |
| 141 | B | A | C |
| 142 | B | A | C |
| 143 | C | B | C |
| 144 | C | A | D |
| 145 | D | B | D |
| 146 | B | A | B |
| 147 | B | A | C |
| 149 | A | A | B |
| 150 | B | A | B |
| 151 | A | A | A |
| 154 | B | A | B |
| 155 | B | A | C |
| 156 | A | A | B |
| 157 | B | A | B |
| 158 | B | B | B |
| 160 | C | B | D |
| 161 | D | D | D |
| 162 | A | A | B |
| 163 | C | C | C |
| 164 | C | C | C |
| 165 | A | A | B |
| 166 | B | B | C |
| 167 | B | B | C |
| 168 | C | B | D |
| 169 | C | C | C |
| 170 | C | C | C |
| 171 | B | B | C |
| 172 | B | B | C |
| 173 | C | C | D |
| 174 | D | C | D |
| 175 | D | D | D |
| 176 | D | D | D |
| 177 | D | D | D |
| 178 | C | C | C |
| 180 | D | D | D |
| 181 | C | C | D |
| 182 | A | A | B |
| 183 | B | B | C |
| 184 | C | B | D |
| 187 | A | A | B |
| 188 | B | B | C |
| 189 | D | C | D |
| 190 | B | | |
| 191 | D | D | D |
| 192 | A | A | A |
| 193 | A | A | A |
| 195 | B | B | B |
| 196 | C | C | C |
| 197 | B | B | C |
| 198 | A | A | C |
| 200 | D | C | D |
| 201 | D | D | D |
| 202 | D | C | C |
| 203 | D | C | D |
| 204 | C | C | C |
| 205 | C | C | C |
| 206 | A | A | B |
| 207 | B | A | C |
| 208 | B | A | B |
| 209 | B | B | C |
| 210 | A | A | B |
| 211 | C | B | C |
| 212 | C | B | D |
| 213 | C | C | D |
| 214 | C | B | C |
| 215 | B | A | B |
| 216 | A | A | B |
| 218 | A | A | B |
| 219 | D | C | D |
| 220 | C | C | C |
| 221 | D | D | D |
| 222 | C | A | C |
| 223 | C | B | C |
| 224 | D | D | D |
| 225 | A | A | B |
| 226 | A | A | B |
| 231 | C | B | D |
| 233 | C | A | C |
| 235 | C | B | C |
| 236 | B | B | C |
| 237 | D | D | D |
| 238 | A | A | B |

### EQUIVALENTS AND SCOPE

This application refers to various issued patents, published patent applications, journal articles, and other publications, all of which are incorporated herein by reference. If there is a conflict between any of the incorporated references and the instant specification, the specification shall control. In addition, any particular embodiment of the present invention that falls within the prior art may be explicitly excluded from any one or more of the claims. Because such embodiments are deemed to be known to one of ordinary skill in the art, they may be excluded even if the exclusion is not set forth explicitly herein. Any particular embodiment of the invention can be excluded from any claim, for any reason, whether or not related to the existence of prior art.

Those skilled in the art will recognize or be able to ascertain using no more than routine experimentation many equivalents to the specific embodiments described herein. The scope of the present embodiments described herein is not intended to be limited to the above Description, FIGURES, or Examples but rather is as set forth in the appended claims. Those of ordinary skill in the art will appreciate that various changes and modifications to this description may be made without departing from the spirit or scope of the present invention, as defined in the following claims.

1. A compound of Formula (I-a): 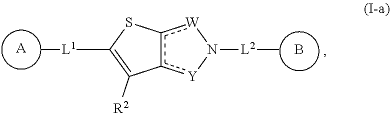
or a pharmaceutically acceptable salt, solvate, hydrate, tautomer, or stereoisomer thereof, wherein:
A and B are each independently heterocyclyl or heteroaryl, each of which is optionally substituted with one or more R¹;
L¹ and L² are each independently absent, C₁-C₆-alkylene, C₁-C₆-heteroalkylene, C₆-C₁₂-arylene, C₅-C₁₂-heteroarylene, —O—, —C(O)—, —N(R³)—, —N(R³)C(O)—, or —C(O)N(R³)—, wherein each alkylene, heteroalkylene, arylene, and heteroarylene is optionally substituted with one or more R⁴;
W and Y are each independently C(R⁵) or N;
wherein at least one of W and Y is N, and the dashed lines in the ring comprising W, N, and Y may be single or double bonds as valency permits;
each R¹ is independently hydrogen, C₁-C₆-alkyl, C₂-C₆-alkenyl, C₂-C₆-alkynyl, C₁-C₆-heteroalkyl, C₁-C₆-haloalkyl, cycloalkyl, heterocyclyl, aryl, heteroaryl, C₁-C₆ alkylene-cycloalkyl, C₁-C₆ alkylene-heterocyclyl, C₁-C₆ alkylene-aryl, C₂-C₆ alkenylene-aryl, C₁-C₆ alkylene-heteroaryl, halo, cyano, oxo, —OR^{A}, —NR^{B}R^{C}, —NR^{B}C(O)R^{D}, —NO₂, —C(O)NR^{B}R^{C}, —C(O)R^{D}, —C(O)OR^{D} or —S(O)ₓR^{D}, wherein each alkyl, alkylene, alkenyl, alkenylene, alkynyl, heteroalkyl, haloalkyl, cycloalkyl, heterocyclyl, aryl, and heteroaryl is optionally substituted with one or more R⁶; or
two R¹ groups, together with the atoms to which they are attached, form a 3-7-membered cycloalkyl, heterocyclyl, aryl, or heteroaryl, wherein each cycloalkyl, heterocyclyl, aryl, and heteroaryl is optionally substituted with one or more R⁶;
R² is hydrogen, halo, cyano, C₁-C₆-alkyl, C₂-C₆-alkenyl, C₂-C₆-alkynyl, C₁-C₆-heteroalkyl, C₁-C₆-haloalkyl, or —OR^{A};
each R³ is independently hydrogen, C₁-C₆-alkyl, or C₁-C₆-haloalkyl;
each R⁴ is independently C₁-C₆-alkyl, C₁-C₆-heteroalkyl, C₁-C₆-haloalkyl, cycloalkyl, halo, cyano, oxo, —OR^{A}, —NR^{B}R^{C}, —C(O)R^{D}, or —C(O)OR^{D};
R⁵ is hydrogen, C₁-C₆-alkyl, C₂-C₆-alkenyl, C₂-C₆-alkynyl, C₁-C₆-heteroalkyl, C₁-C₆-haloalkyl, halo, cyano, or —OR^{A};
each R⁶ is independently C₁-C₆-alkyl, C₂-C₆-alkenyl, C₂-C₆-alkynyl, C₁-C₆-heteroalkyl, C₁-C₆-haloalkyl, cycloalkyl, heterocyclyl, aryl, heteroaryl, halo, cyano, oxo, —OR^{A}, —NR^{B}R^{C}, —NR^{B}C(O)R^{D}, —NO₂, —C(O)NR^{B}R^{C}, —C(O)R^{D}, —C(O)OR^{D}, or —S(O)ₓR^{D}, wherein each of alkyl, alkenyl, alkynyl, heteroalkyl, haloalkyl, cycloalkyl, heterocyclyl, aryl, and heteroaryl is optionally substituted with one or more R⁷;
each R^{A} is independently hydrogen, C₁-C₆ alkyl, C₁-C₆ heteroalkyl, C₁-C₆ haloalkyl, cycloalkyl, heterocyclyl, aryl, heteroaryl, C₁-C₆ alkylene-aryl, C₁-C₆ alkylene-heteroaryl, —C(O)R^{D} or —S(O)ₓR^{D}, wherein each alkyl, alkylene, heteroalkyl, haloalkyl, cycloalkyl, heterocyclyl, aryl, and heteroaryl is optionally substituted with one or more R⁸;
each R^{B} and R^{C} is independently hydrogen, C₁-C₆ alkyl, C₁-C₆ heteroalkyl, C₁-C₆ haloalkyl, cycloalkyl, heterocyclyl, aryl, heteroaryl, C₁-C₆ alkylene-cycloalkyl, C₁-C₆ alkylene-heterocyclyl, —OR^{A}, wherein each alkyl, alkylene, heteroalkyl, haloalkyl, cycloalkyl, heterocyclyl, aryl, and heteroaryl is optionally substituted with one or more R⁸; or
R^{B} and R^{C} together with the atom to which they are attached form a 3-7-membered heterocyclyl ring optionally substituted with one or more R⁸;
each R^{D} is independently hydrogen, C₁-C₆ alkyl, C₂-C₆ alkenyl, C₂-C₆ alkynyl, C₁-C₆ heteroalkyl, C₁-C₆ haloalkyl, cycloalkyl, heterocyclyl, aryl, heteroaryl, C₁-C₆ alkylene-aryl, or C₁-C₆ alkylene-heteroaryl, wherein each alkyl, alkylene, alkenyl, alkynyl, heteroalkyl, haloalkyl, cycloalkyl, heterocyclyl, aryl, and heteroaryl is optionally substituted with one or more R⁸;
each R⁷ is independently C₁-C₆ alkyl, C₁-C₆ heteroalkyl, C₁-C₆ haloalkyl, cycloalkyl, heterocyclyl, aryl, heteroaryl, halo, cyano, oxo, or —OR^{A};
each R⁸ is C₁-C₆-alkyl, halo, cyano, oxo, or —OR^{A1};
each R^{A}1 is hydrogen or C₁-C₆-alkyl; and
x is 0, 1, or 2.

2. The compound of claim 1, wherein one of A and B is independently a monocyclic heteroaryl or bicyclic heteroaryl, each of which is optionally substituted with one or more R¹.

3. (canceled)

4. The compound of claim 1, wherein one of A and B is independently a nitrogen-containing heteroaryl optionally substituted with one or more R¹.

5. (canceled)

6. The compound of claim 1, wherein one of A and B is independently selected from 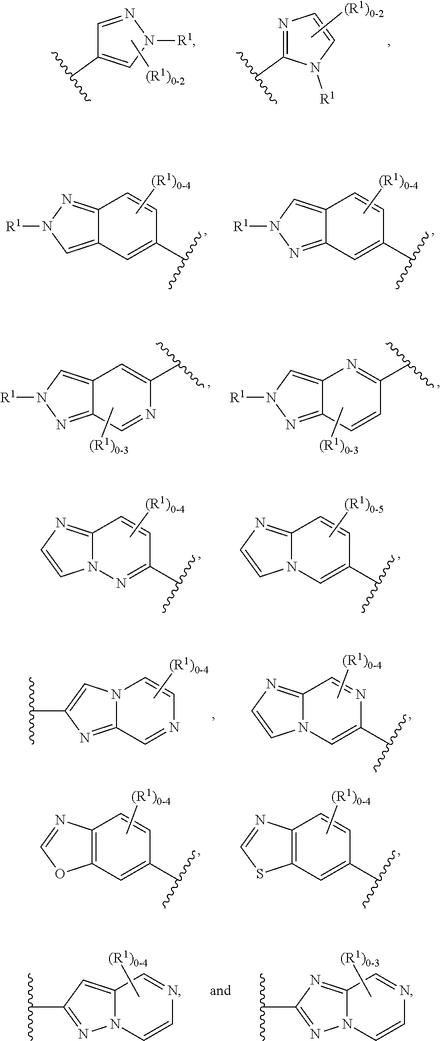
wherein R¹ is as described in claim 1.

7-8. (canceled)

9. The compound of claim 1, wherein one of A and B is independently selected from 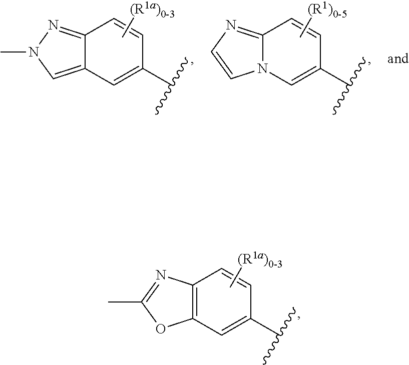
wherein each R^{1a} is independently C₁-C₆-alkyl, C₁-C₆-heteroalkyl, C₁-C₆-haloalkyl, halo, cyano, or —OR^{A}, and each alkyl, heteroalkyl, and haloalkyl is optionally substituted with one or more R⁷.

10. The compound of claim 1, wherein one of A and B is independently 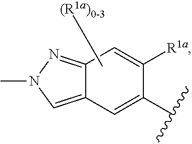
wherein each R^{1a} is independently C₁-C₆-alkyl, C₁-C₆-heteroalkyl, C₁-C₆-haloalkyl, halo, cyano, or —OR^{A}, and each alkyl, heteroalkyl, and haloalkyl is optionally substituted with one or more R⁷.

11. The compound of claim 10, wherein at least one of R^{1a} is C₁-C₆-alkyl, halo, or —OR^{A}.

12. (canceled)

13. The compound of claim 1, wherein A is selected from 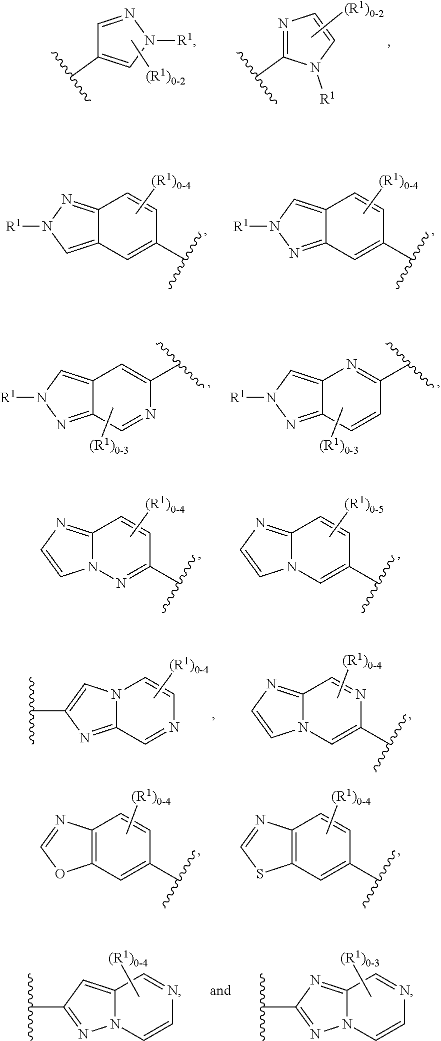
wherein R¹ is as described in claim 1.

14. The compound of claim 1, wherein A is 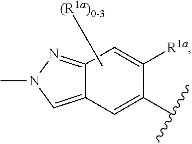
wherein each R^{1a} is independently C₁-C₆-alkyl, C₁-C₆-heteroalkyl, C₁-C₆-haloalkyl, halo, cyano, or —OR^{A}, and each alkyl, heteroalkyl, and haloalkyl is optionally substituted with one or more R⁷.

15. The compound of claim 14, wherein at least one of R^{1a} is C₁-C₆-alkyl, halo, or —OR^{A}.

16. (canceled)

17. The compound of claim 1, wherein B is selected from 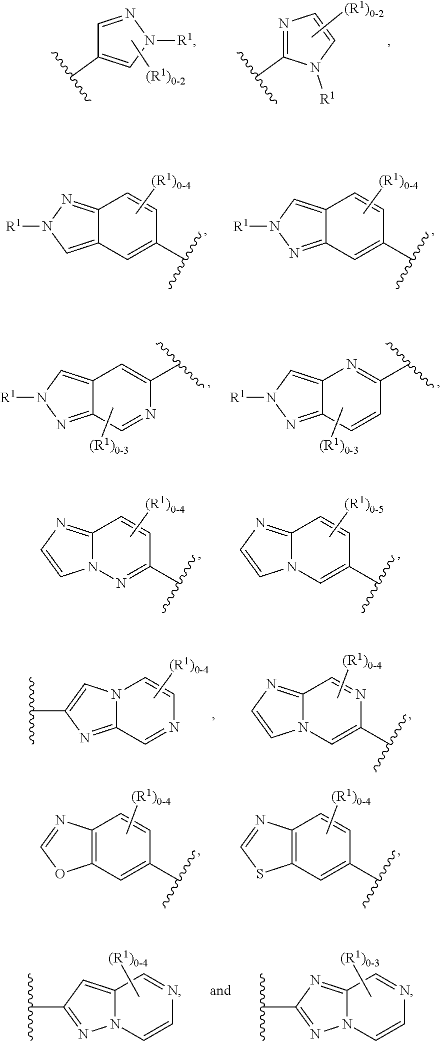
wherein R¹ is as described in claim 1.

18. The compound of claim 1, wherein B is 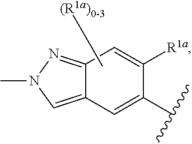
wherein each R^{1a} is independently C₁-C₆-alkyl, C₁-C₆-heteroalkyl, C₁-C₆-haloalkyl, halo, cyano, or —OR^{A}, and each alkyl, heteroalkyl, and haloalkyl is optionally substituted with one or more R⁷.

19. The compound of claim 18, wherein at least one of R^{1a} is C₁-C₆-alkyl, halo, or —OR^{A}.

20. (canceled)

21. The compound of claim 1, wherein one of A and B is independently selected from

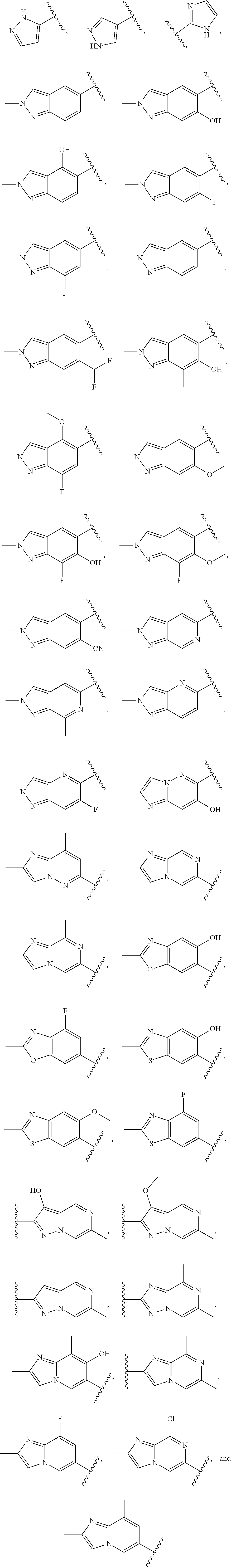

22. (canceled)

23. The compound of claim 1, wherein one of A and B is independently selected from

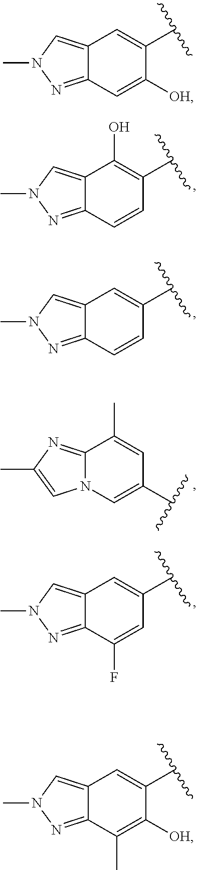

24. The compound of claim 1, wherein one of A and B is independently

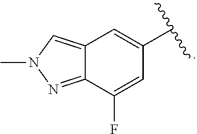 [SMILES: *c1cc(F)c2nn(C)cc2c1]

25-27. (canceled)

28. The compound of claim 1, wherein one of A and B is independently a nitrogen-containing heterocyclyl optionally substituted with one or more R¹.

29. (canceled)

30. The compound of claim 1, wherein one of A and B is independently selected from 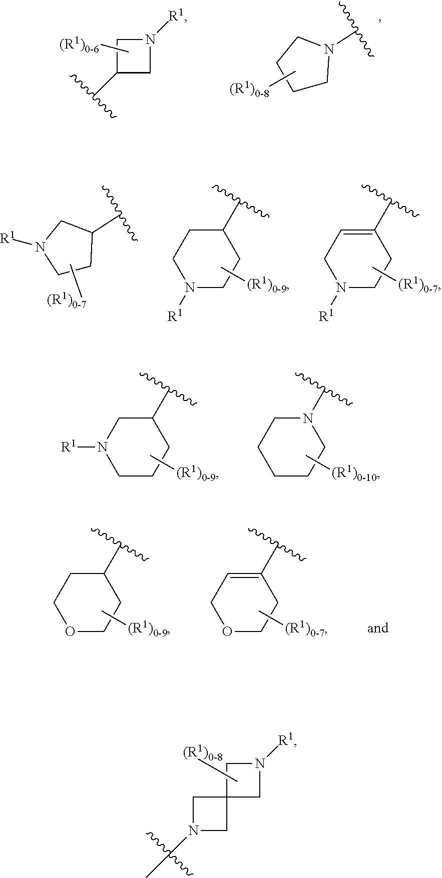
wherein R¹ is as described in claim 1.

31. The compound of claim 1, wherein one of A and B is independently selected from 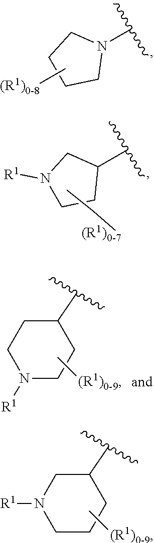
and, wherein R¹ is as described in claim 1.

32. The compound of claim 1, wherein one of A and B is 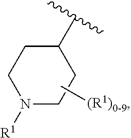
wherein R¹ is as described in claim 1.

33-34. (canceled)

35. The compound of claim 1, wherein A is selected from 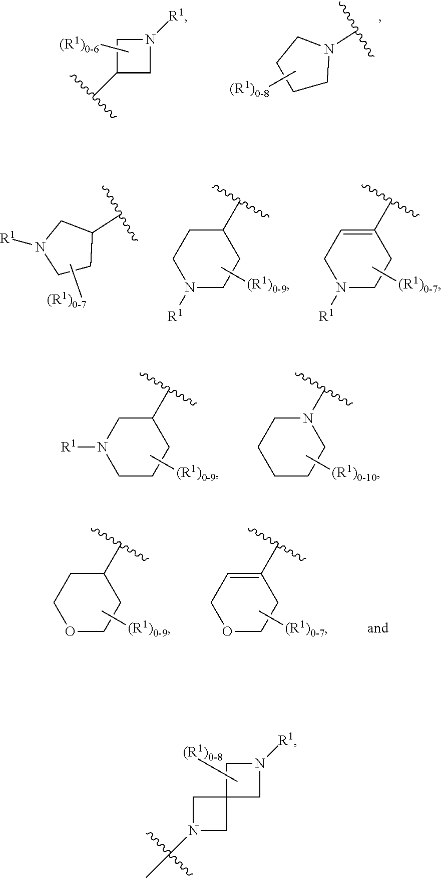
wherein R¹ is as described in claim 1.

36. The compound of claim 1, wherein B is selected from 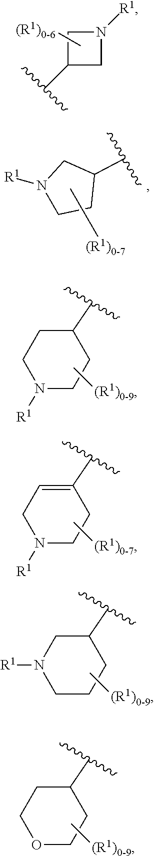
wherein R¹ is as described in claim 1.

37. The compound of claim 1, wherein one of A and B is independently is selected from

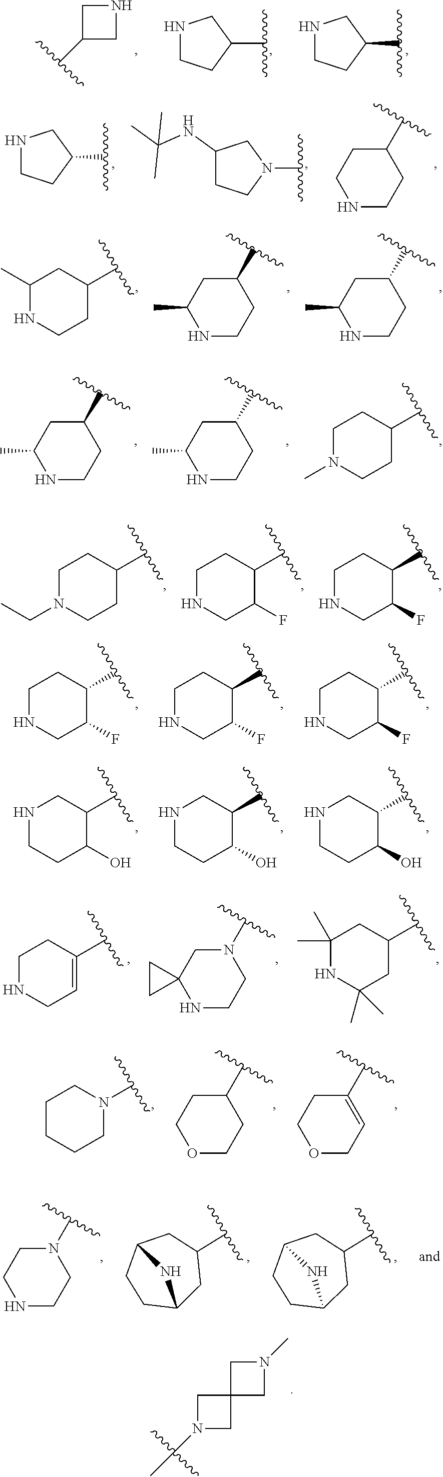

38. (canceled)

39. The compound of claim 1, wherein one of A and B is

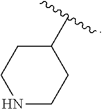 [SMILES: *C1CCNCC1]

40-41. (canceled)

42. The compound of claim 1, wherein each of L¹ and L² is independently absent, —N(R³)— (e.g., —N(CH₃)—), or C₆-C₁₂-arylene, wherein arylene is optionally substituted with one or more R¹.

43. (canceled)

44. The compound of claim 1, wherein each of L¹ and L² is independently absent.

45. The compound of claim 1, wherein one of W and Y is independently N.

46-49. (canceled)

50. The compound of claim 1, wherein the compound of Formula (I) is a compound of Formula (I-d): 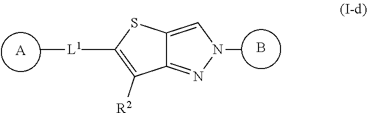
or a pharmaceutically acceptable salt, solvate, hydrate, tautomer, or stereoisomer thereof, wherein A, B, R², and subvariables thereof are as defined in claim 1.

51. The compound of claim 1, wherein the compound of Formula (I) is a compound of Formula (I-e): 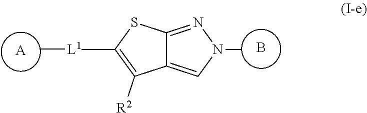
or a pharmaceutically acceptable salt, solvate, hydrate, tautomer, or stereoisomer thereof, wherein A, B, R², and subvariables thereof are as defined in claim 1.

52-53. (canceled)

54. The compound of claim 1, wherein the compound of Formula (I) is a compound of Formula (I-g): 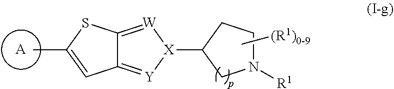
or a pharmaceutically acceptable salt, solvate, hydrate, tautomer, or stereoisomer thereof, wherein p is 0, 1, 2, or 3; X is C, C(R⁵), or N; and A, W, Y, R¹ and subvariables thereof are as defined in claim 1.

55. (canceled)

56. The compound of claim 1, wherein the compound of Formula (I) is a compound of Formula (I-i): 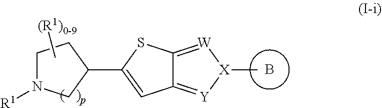
or a pharmaceutically acceptable salt, solvate, hydrate, tautomer, or stereoisomer thereof, wherein p is 0, 1, 2, or 3; X is C, C(R⁵), or N; and B, W, Y, R¹ and subvariables thereof are as defined in claim 1.

57. (canceled)

58. The compound of claim 1, wherein the compound of Formula (I) is a compound of Formula (I-j): 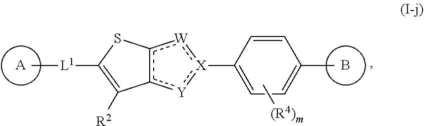
or a pharmaceutically acceptable salt, solvate, hydrate, tautomer, or stereoisomer thereof, wherein m is 0, 1, 2, 3, or 4; X is C, C(R⁵), or N; and A, B, L¹, W, Y, R², and subvariables thereof are as defined in claim 1.

59. A compound of Formula (III): 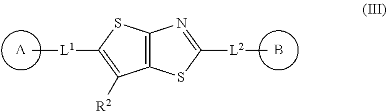
or a pharmaceutically acceptable salt, solvate, hydrate, tautomer, or stereoisomer thereof, wherein:
A and B are each independently heterocyclyl or heteroaryl, each of which is optionally substituted with one or more R¹;
L¹ and L² are each independently absent, C₁-C₆-alkylene, C₁-C₆-heteroalkylene, —O—, —C(O)—, —N(R³)—, —N(R³)C(O)—, or —C(O)N(R³)—, wherein each alkylene, heteroalkylene, arylene, and heteroarylene is optionally substituted with one or more R⁴;
each R¹ is independently hydrogen, C₁-C₆-alkyl, C₂-C₆-alkenyl, C₂-C₆-alkynyl, C₁-C₆-heteroalkyl, C₁-C₆-haloalkyl, cycloalkyl, heterocyclyl, aryl, C₁-C₆ alkylene-cycloalkyl, C₁-C₆ alkylene-heterocyclyl, C₁-C₆ alkylene-aryl, C₁-C₆ alkenylene-aryl, C₁-C₆ alkylene-heteroaryl, heteroaryl, halo, cyano, oxo, —OR^{A}, —NR^{B}R^{C}, —NR^{B}C(O)R^{D}, —NO₂, —C(O)NR^{B}R^{C}, —C(O)R^{D}, —C(O)OR^{D}, or —S(O)ₓR^{D}, wherein each alkyl, alkylene, alkenyl, alkynyl, heteroalkyl, haloalkyl, cycloalkyl, heterocyclyl, aryl, and heteroaryl is optionally substituted with one or more R⁶; or
two R¹ groups, together with the atoms to which they are attached, form a 3-7-membered cycloalkyl, heterocyclyl, aryl, or heteroaryl, wherein each cycloalkyl, heterocyclyl, aryl, and heteroaryl is optionally substituted with one or more R⁶;
R² is hydrogen, halo, cyano, C₁-C₆-alkyl, C₂-C₆-alkenyl, C₂-C₆-alkynyl, C₁-C₆-heteroalkyl, or C₁-C₆-haloalkyl;
each R³ is independently hydrogen, C₁-C₆-alkyl, or C₁-C₆-haloalkyl;
each R⁴ is independently C₁-C₆-alkyl, C₁-C₆-heteroalkyl, C₁-C₆-haloalkyl, cycloalkyl, halo, cyano, oxo, —OR^{A}, —NR^{B}R^{C}, —C(O)R^{D}, or —C(O)OR^{D};
each R⁶ is independently C₁-C₆-alkyl, C₂-C₆-alkenyl, C₂-C₆-alkynyl, C₁-C₆-heteroalkyl, C₁-C₆-haloalkyl, cycloalkyl, heterocyclyl, aryl, heteroaryl, halo, cyano, oxo, —OR^{A}, —NR^{B}R^{C}, —NR^{B}C(O)R^{D}, —NO₂, —C(O)NR^{B}R^{C}, —C(O)R^{D}, —C(O)OR^{D}, or —S(O)ₓR^{D}, wherein each of alkyl, alkenyl, alkynyl, heteroalkyl, haloalkyl, cycloalkyl, heterocyclyl, aryl, and heteroaryl is optionally substituted with one or more R⁷;
each R^{A} is independently hydrogen, C₁-C₆ alkyl, C₁-C₆ heteroalkyl, C₁-C₆ haloalkyl, cycloalkyl, heterocyclyl, aryl, heteroaryl, C₁-C₆ alkylene-aryl, C₁-C₆ alkylene-heteroaryl, —C(O)R^{D} or —S(O)ₓR^{D}, wherein each alkyl, alkylene, heteroalkyl, haloalkyl, cycloalkyl, heterocyclyl, aryl, and heteroaryl is optionally substituted with one or more R⁸;
each R^{B} and R^{C} is independently hydrogen, C₁-C₆ alkyl, C₁-C₆ heteroalkyl, C₁-C₆ haloalkyl, cycloalkyl, heterocyclyl, aryl, heteroaryl, C₁-C₆ alkylene-cycloalkyl, C₁-C₆ alkylene-heterocyclyl, —OR^{A}, wherein each alkyl, alkylene, heteroalkyl, haloalkyl, cycloalkyl, heterocyclyl, aryl, and heteroaryl is optionally substituted with one or more R⁸; or
R^{B} and R^{C} together with the atom to which they are attached form a 3-7-membered heterocyclyl ring optionally substituted with one or more R⁸;
each R^{D} is independently hydrogen, C₁-C₆ alkyl, C₂-C₆ alkenyl, C₂-C₆ alkynyl, C₁-C₆ heteroalkyl, C₁-C₆ haloalkyl, cycloalkyl, heterocyclyl, aryl, heteroaryl, C₁-C₆ alkylene-aryl, or C₁-C₆ alkylene-heteroaryl, wherein each alkyl, alkylene, alkenyl, alkynyl, heteroalkyl, haloalkyl, cycloalkyl, heterocyclyl, aryl, and heteroaryl is optionally substituted with one or more R⁸;
each R⁷ is independently C₁-C₆ alkyl, C₁-C₆ heteroalkyl, C₁-C₆ haloalkyl, cycloalkyl, heterocyclyl, aryl, heteroaryl, halo, cyano, oxo, or —OR^{A};
each R⁸ is C₁-C₆-alkyl, halo, cyano, oxo, or —OR^{A1};
each R^{A1} is hydrogen or C₁-C₆-alkyl; and
x is 0, 1, or 2.

60. The compound of claim 59, wherein one of A and B is independently a monocyclic heteroaryl or bicyclic heteroaryl, each of which is optionally substituted with one or more R¹.

61. (canceled)

62. The compound of claim 59, wherein one of A and B is independently a nitrogen-containing heteroaryl optionally substituted with one or more R¹.

63. The compound of claim 59, wherein one of A and B is independently selected from 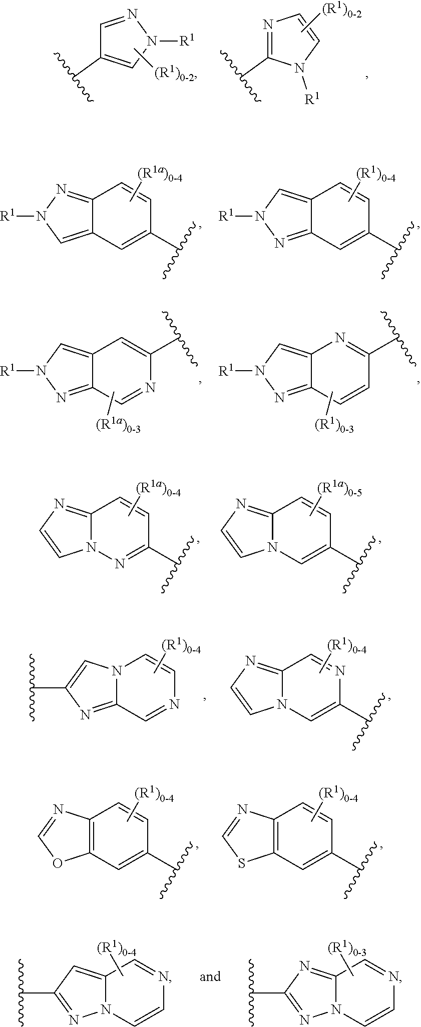
wherein R¹ is as described in claim 59.

64. (canceled)

65. The compound of claim 59, wherein one of A and B is independently selected from 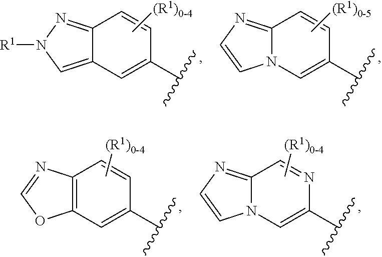
wherein R¹ is as described in claim 59.

66. (canceled)

67. The compound of claim 59, wherein one of A and B is independently 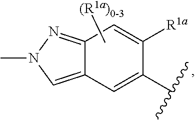
wherein each R^{1a} is independently C₁-C₆-alkyl, C₁-C₆-heteroalkyl, C₁-C₆-haloalkyl, halo, cyano, or —OR^{A}, and each alkyl, heteroalkyl, and haloalkyl is optionally substituted with one or more R⁷.

68. The compound of claim 67, wherein at least one of R^{1a} is C₁-C₆-alkyl, halo, or —OR^{A}.

69-70. (canceled)

71. The compound of claim 59, wherein B is 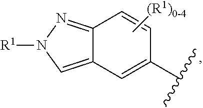
wherein R¹ is as described in claim 59.

72. The compound of claim 59, wherein one of A and B is independently selected from

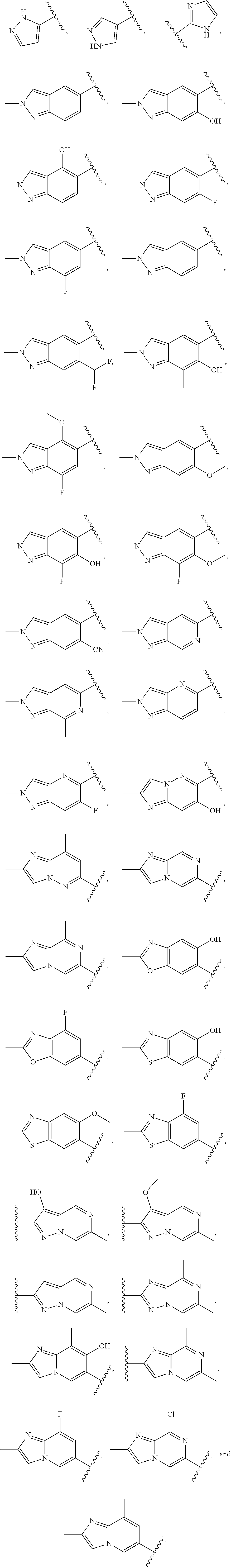

73. (canceled)

74. The compound of claim 59, wherein one of A and B is independently selected from

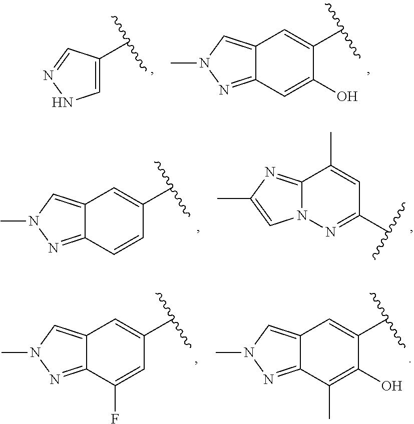

75. The compound of claim 59, wherein one of A and B is independently

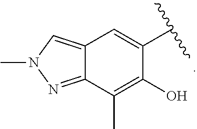 [SMILES: *c1cc2cn(C)nc2c(C)c1O]

76-78. (canceled)

79. The compound of claim 59, wherein one of A and B is independently a nitrogen-containing heterocyclyl optionally substituted with one or more R¹.

80. The compound of claim 59, wherein one of A and B is independently selected from 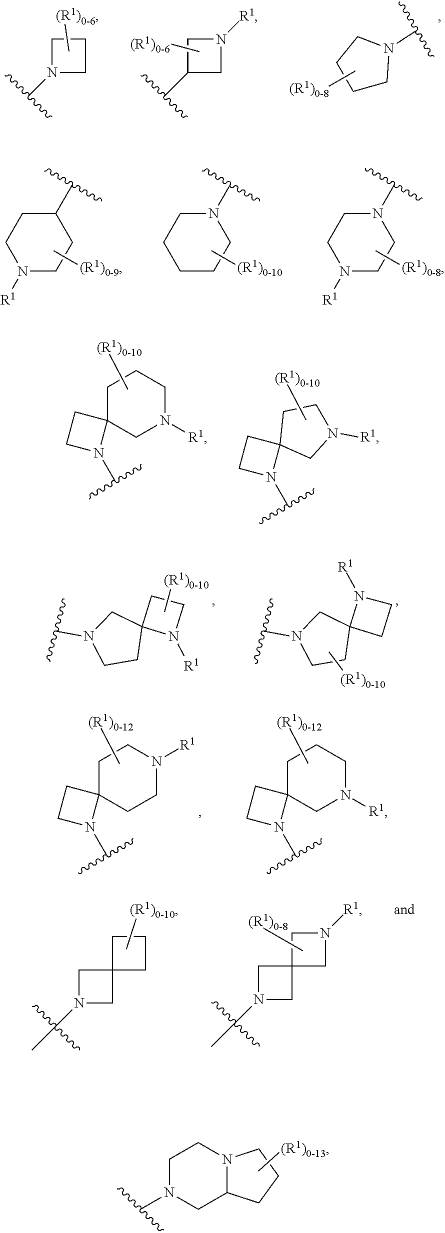
and Wherein R¹ is as described in claim 59.

81. (canceled)

82. The compound of claim 59, wherein one of A and B is 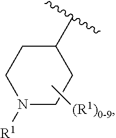
wherein R¹ is as described in claim 59.

83-84. (canceled)

85. The compound of claim 59, wherein one of A and B is independently is selected

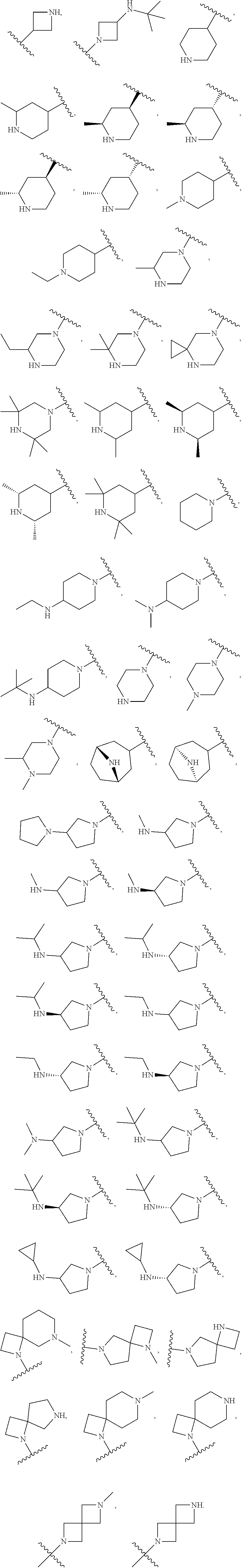

86. (canceled)

87. The compound of claim 59, wherein one of A and B is

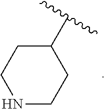 [SMILES: *C1CCNCC1]

88-89. (canceled)

90. The compound of claim 59, wherein each of L¹ and L² is independently absent, —N(R³)— (e.g., —N(CH₃)—), or C₆-C₁₂-arylene, wherein arylene is optionally substituted with one or more R¹.

91-92. (canceled)

93. The compound of claim 59, wherein R² is hydrogen or halo (e.g., hydrogen).

94. The compound of claim 59, wherein the compound of Formula (III) is a compound of Formula (III-a): 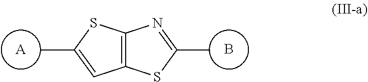
or a pharmaceutically acceptable salt, solvate, hydrate, tautomer, or stereoisomer thereof, wherein A, B, and subvariables thereof are as defined in claim 59.

95. The compound of claim 59, wherein the compound of Formula (III) is a compound of Formula (III-b): 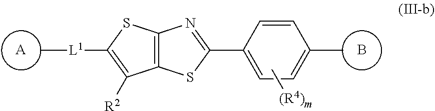
or a pharmaceutically acceptable salt, solvate, hydrate, tautomer, or stereoisomer thereof, wherein m is 0, 1, 2, 3, or 4, and A, B, L¹, R², R⁴, and subvariables thereof are as defined in claim 59.

96. The compound of claim 59, wherein the compound of Formula (III) is a compound of Formula (III-c): 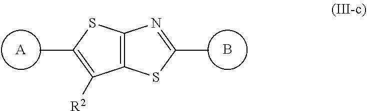
or a pharmaceutically acceptable salt, solvate, hydrate, tautomer, or stereoisomer thereof, wherein A, B, and R², and subvariables thereof are as defined in claim 59.

97. A compound of Formula (IV): 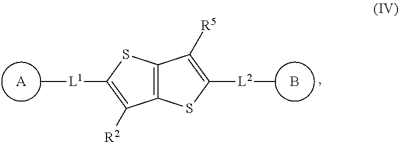
or a pharmaceutically acceptable salt, solvate, hydrate, tautomer, or stereoisomer thereof, wherein:
A and B are each independently cycloalkyl, heterocyclyl, aryl, or heteroaryl, each of which is optionally substituted with one or more R¹;
L¹ and L² are each independently absent, C₁-C₆-alkylene, C₁-C₆-heteroalkylene, C₆-C₁₂-arylene, C₅-C₁₂-heteroarylene, —O—, —C(O)—, —N(R³)—, —N(R³)C(O)—, or —C(O)N(R³)—, wherein each alkylene, heteroalkylene, arylene, and heteroarylene is optionally substituted with one or more R⁴;
each R¹ is independently hydrogen, C₁-C₆-alkyl, C₂-C₆-alkenyl, C₂-C₆-alkynyl, C₁-C₆-heteroalkyl, C₁-C₆-haloalkyl, cycloalkyl, heterocyclyl, aryl, C₁-C₆ alkylene-cycloalkyl, C₁-C₆ alkylene-heterocyclyl, C₁-C₆ alkylene-aryl, C₁-C₆ alkenylene-aryl, C₁-C₆ alkylene-heteroaryl, heteroaryl, halo, cyano, oxo, —OR^{A}, —NR^{B}R^{C}, —NR^{B}C(O)R^{D}, —NO₂, —C(O)NR^{B}R^{C}, —C(O)R^{D}, —C(O)OR^{D}, or —S(O)ₓR^{D}, wherein each alkyl, alkylene, alkenyl, alkynyl, heteroalkyl, haloalkyl, cycloalkyl, heterocyclyl, aryl, and heteroaryl is optionally substituted with one or more R⁶; or
two R¹ groups, together with the atoms to which they are attached, form a 3-7-membered cycloalkyl, heterocyclyl, aryl, or heteroaryl, wherein each cycloalkyl, heterocyclyl, aryl, and heteroaryl is optionally substituted with one or more R⁶;
R² and R⁵ are each independently hydrogen, halo, cyano, C₁-C₆-alkyl, C₂-C₆-alkenyl, C₂-C₆-alkynyl, C₁-C₆-heteroalkyl, or C₁-C₆-haloalkyl;
each R³ is independently hydrogen, C₁-C₆-alkyl, or C₁-C₆-haloalkyl;
each R⁴ is independently C₁-C₆-alkyl, C₁-C₆-heteroalkyl, C₁-C₆-haloalkyl, cycloalkyl, halo, cyano, oxo, —OR^{A}, —NR^{B}R^{C}, —C(O)R^{D}, or —C(O)OR^{D};
each R⁶ is independently C₁-C₆-alkyl, C₂-C₆-alkenyl, C₂-C₆-alkynyl, C₁-C₆-heteroalkyl, C₁-C₆-haloalkyl, cycloalkyl, heterocyclyl, aryl, heteroaryl, halo, cyano, oxo, —OR^{A}, —NR^{B}R^{C}, —NR^{B}C(O)R^{D}, —NO₂, —C(O)NR^{B}R^{C}, —C(O)R^{D}, —C(O)OR^{D}, or —S(O)ₓR^{D}, wherein each of alkyl, alkenyl, alkynyl, heteroalkyl, haloalkyl, cycloalkyl, heterocyclyl, aryl, and heteroaryl is optionally substituted with one or more R⁷;
each R^{A} is independently hydrogen, C₁-C₆ alkyl, C₁-C₆ heteroalkyl, C₁-C₆ haloalkyl, cycloalkyl, heterocyclyl, aryl, heteroaryl, C₁-C₆ alkylene-aryl, C₁-C₆ alkylene-heteroaryl, —C(O)R^{D} or —S(O)ₓR^{D}, wherein each alkyl, alkylene, heteroalkyl, haloalkyl, cycloalkyl, heterocyclyl, aryl, and heteroaryl is optionally substituted with one or more R⁸;
each R^{B} and R^{C} is independently hydrogen, C₁-C₆ alkyl, C₁-C₆ heteroalkyl, C₁-C₆ haloalkyl, cycloalkyl, heterocyclyl, aryl, heteroaryl, C₁-C₆ alkylene-cycloalkyl, C₁-C₆ alkylene-heterocyclyl, —OR^{A}, wherein each alkyl, alkylene, heteroalkyl, haloalkyl, cycloalkyl, heterocyclyl, aryl, and heteroaryl is optionally substituted with one or more R⁸; or
R^{B} and R^{C} together with the atom to which they are attached form a 3-7-membered heterocyclyl ring optionally substituted with one or more R⁸;
each R^{D} is independently hydrogen, C₁-C₆ alkyl, C₂-C₆ alkenyl, C₂-C₆ alkynyl, C₁-C₆ heteroalkyl, C₁-C₆ haloalkyl, cycloalkyl, heterocyclyl, aryl, heteroaryl, C₁-C₆ alkylene-aryl, or C₁-C₆ alkylene-heteroaryl, wherein each alkyl, alkylene, alkenyl, alkynyl, heteroalkyl, haloalkyl, cycloalkyl, heterocyclyl, aryl, and heteroaryl is optionally substituted with one or more R⁸;
each R⁷ is independently C₁-C₆ alkyl, C₁-C₆ heteroalkyl, C₁-C₆ haloalkyl, cycloalkyl, heterocyclyl, aryl, heteroaryl, halo, cyano, oxo, or —OR^{A};
each R⁸ is C₁-C₆-alkyl, halo, cyano, oxo, or —OR^{A1};
each R^{A}1 is hydrogen or C₁-C₆-alkyl; and
x is 0, 1, or 2.

98. The compound of claim 1, wherein the compound is selected from any one of the compounds shown in any one of Tables 1-4 or a pharmaceutically acceptable salt, solvate, hydrate, tautomer, or stereoisomer thereof.

99. A pharmaceutical composition comprising a compound of claim 1 and a pharmaceutically acceptable excipient.

100. The compound of claim 1, wherein the compound:
(i) alters a target nucleic acid;
(ii) binds to a target nucleic acid; or
(iii) stabilizes a target nucleic acid.

101-102. (canceled)

103. The compound of claim 1, wherein the compound:
(i) increases splicing at splice site on a target nucleic acid, by about 0.5%, 1%, 2%, 3%, 4%, 5%, 6%, 7%, 8%, 9%, 10%, 15%, 20%, 25%, 30%, 35%, 40%, 45%, 50%, 55%, 60%, 65%, 70%, 75%, 80%, 85%, 90%, 95%, or more, e.g., as determined by qPCR; or
(ii) decreases splicing at splice site on a target nucleic acid, by about 0.5%, 1%, 2%, 3%, 4%, 5%, 6%, 7%, 8%, 9%, 10%, 15%, 20%, 25%, 30%, 35%, 40%, 45%, 50%, 55%, 60%, 65%, 70%, 75%, 80%, 85%, 90%, 95%, or more, e.g., as determined by qPCR.

104. (canceled)

105. A method of modulating splicing of a nucleic acid comprising contacting the nucleic acid with a compound of Formula (I), (II), (III), (IV), as described in claim 1.

106. The method of claim 105, wherein the compound:
i) increases splicing at splice site on a target nucleic acid, by about 0.5%1, %, 2%, 3%, 4%, 5%, 6%, 7%, 8%, 9%, 10%, 15%, 20%, 25%, 30%, 35%, 40%, 45%, 50%, 55%, 60%, 65%, 70%, 75%, 80%, 85%, 90%, 95%, or more, e.g., as determined by qPCR; or
(ii) decreases splicing at splice site on a target nucleic acid, by about 0.5%, 1%, 2%, 3%, 4%, 5%, 6%, 7%, 8%, 9%, 10%, 15%, 20%, 25%, 30%, 35%, 40%, 45%, 50%, 55%, 60%, 65%, 70%, 75%, 80%, 85%, 90%, 95%, or more, e.g., as determined by gPCR.

107. (canceled)

108. A method of forming a complex comprising a component of a spliceosome (e.g., a major spliceosome component or a minor spliceosome component), a nucleic acid (e.g., a DNA, RNA, e.g., a pre-mRNA), and a compound of Formula (I), (II), (III), or (IV):
comprising contacting the nucleic acid (e.g., a DNA, RNA, e.g., a pre-mRNA) with a compound of Formula (I), (II), (III), or (IV), according to claim 1.

109. The method of claim 108, wherein the component of a spliceosome is recruited to the nucleic acid in the presence of the compound of Formula (I), (II), (III), or (IV).

110. A method of altering the conformation of a nucleic acid (e.g., a DNA, RNA, e.g., a pre-mRNA) comprising contacting the nucleic acid with a compound of Formula (I), (II), (III), or (IV), according to claim 1.

111. The method of claim 110, wherein the altering comprises:
(i) forming a bulge in the nucleic acid;
(ii) stabilizing a bulge in the nucleic acid; or
(iii) reducing a bulge in the nucleic acid.

112-113. (canceled)

114. The method of claim 110, wherein the nucleic acid comprises a splice site.

115. A composition for use in treating a disease or disorder in a subject comprising administering to the subject a compound of Formula (I), (II), (III), or (IV), according to claim 1.

116. The composition for use of claim 115, wherein the disease or disorder comprises a proliferative disease (e.g., cancer, a benign neoplasm, or angiogenesis).

117. The composition for use of claim 115, wherein the disease or disorder comprises a neurological disease or disorder, autoimmune disease or disorder, immunodeficiency disease or disorder, lysosomal storage disease or disorder, cardiovascular disease or disorder, metabolic disease or disorder, respiratory disease or disorder, renal disease or disorder, or infectious disease.

118. The composition for use of claim 115, wherein the disease or disorder comprises neurological disease or disorder.

119. The composition for use of claim 115, wherein the disease or disorder comprises Huntington's disease.

120. A method for treating a disease or disorder in a subject comprising administering to the subject a compound of Formula (I), (II), (III), or (IV), according to claim 1.

121. The method of claim 120, wherein the disease or disorder comprises a proliferative disease; or a neurological disease or disorder, autoimmune disease or disorder, immunodeficiency disease or disorder, lysosomal storage disease or disorder, cardiovascular disease or disorder, metabolic disease or disorder, respiratory disease or disorder, renal disease or disorder, or infectious disease.

122-124. (canceled)
